(19) United States
(12) Patent Application Publication
(10) Pub. No.: US 2026/0200926 A1
(43) Pub. Date: Jul. 16, 2026

(54) TREM2 MODULATORS

(71) Applicant: Muna Therapeutics ApS, Frederiksberg (DK)

(72) Inventors: Gavin Whitlock, Sandwich, Kent (GB); Juliette Emmerich, Groningen (NL); Johannes Wilhelm Georg Meissner, Groningen (NL); Jakob Busch-Petersen, Frederiksberg (DK)

(21) Appl. No.: 18/880,872

(22) PCT Filed: Jul. 4, 2023
(86) PCT No.: PCT/EP2023/068406

§ 371 Date: Jan. 3, 2025

(30) Foreign Application Priority Data
Jul. 4, 2022 (EP) 22182841.1
Sep. 19, 2022 (EP) 22196374.7
Dec. 5, 2022 (EP) 22211409.2
Dec. 21, 2022 (EP) 22215488.2
Feb. 2, 2023 (EP) 23154600.3
Mar. 27, 2023 (EP) 23164257.0
Apr. 25, 2023 (EP) 23169796.2
May 10, 2023 (EP) 23172505.2

(51) Int. Cl. C07D 487/04; A61K 31/517; A61K 31/519; A61K 31/5377; A61K 31/5386; A61K 31/55; C07D 405/14; C07D 413/14; C07D 471/04; C07D 519/00

(52) U.S. Cl. CPC C07D 487/04; A61K 31/517; A61K 31/519; A61K 31/5377; A61K 31/5386; A61K 31/55; C07D 405/14; C07D 413/14; C07D 471/04; C07D 519/00

(57) ABSTRACT

The present invention relates to compounds useful for modulating Triggering Receptor Expressed on Myeloid Cells 2 (“TREM2”). The invention also relates to the compounds for use in treatment of conditions related to loss of function of TREM2, such as neurodegenerative diseases and to pharmaceutical compositions comprising the compounds.

### TECHNICAL FIELD

The present invention relates to compounds useful for modulating Triggering Receptor Expressed on Myeloid Cells-2 (“TREM2”). The invention also relates to the compounds for use in treatment of conditions related to loss of function of TREM2, such as neurodegenerative diseases, and to pharmaceutical compositions comprising the compounds.

### BACKGROUND

Triggering receptor expressed on myeloid cells-2 (TREM2) is a transmembrane receptor belonging to the immunoglobulin superfamily and is encoded by the TREM2 gene, which maps to human chromosome 6p21. TREM2 consists of an extracellular part that includes a single immunoglobulin domain and a short ectodomain, a single transmembrane helix and a short cytosolic tail (Colonna, M. et al. (2016))).

Insight to the role of TREM2 is provided by its restricted expression pattern. It is expressed exclusively on myeloid lineage cells, such as macrophages, microglia, dendritic cells and osteoclasts. It plays a role in tissue maintenance, as a sensor of pathology and inducer of innate immune signalling in specific tissues. In the brain TREM2 is exclusively expressed in microglia and is functionally required e.g. in phagocytosis of cellular debris, but has also been assigned roles in restricting inflammation as well as promoting cell survival (Deczkowska, A. et al. (2020)).

TREM2 has a wide range of ligands such as bacterial anionic molecules/endotoxins, phospholipids incl phosphatidylserine, lipoproteins and apolipoproteins incl ApoE, as well as oligomeric Aβ (Hammond, T. R. (2019)).

Signaling via TREM2 is well described through co-receptor DAP12. The adaptor molecule DAP 12 is expressed as a homodimer at the surface of a variety of cells participating in the innate immune response, including microglia, macrophages, granulocytes, NK cells, and dendritic cells. After ligation of TREM2, ITAM (immunoreceptor tyrosine-based activation motif) tyrosine phosphorylation of DAP12 by SRC-family kinases drive the recruitment and activation of the Syk kinase and/or ZAP70 kinase. Downstream of TREM2/DAP12/Syk several signaling pathways have been described involved in cell survival, cell activation and differentiation, and in the control of the actin cytoskeleton.

Proteolytic cleavage of the ectodomain of TREM2 by metalloproteinases, including ADAM10 and ADAM17 and possibly matrix metalloproteinases, leads to the shedding of soluble TREM2 (sTREM2), which can be detected in human cerebrospinal fluid (CSF). sTREM2 has been suggested as a potential biomarker for microglia activity in early-stage Alzheimer's disease (Suárez-Calvet, M. et al. (2016)).

Deficiency of either TREM2 or DAP12 leads to a blunted microglial response to pathological agents. The impact of TREM2-deficiency in vitro has been shown in the context of stimulation with typical TLR ligands, such as LPS. Loss-of-function genetic variants of TREM2 are associated with neurodegenerative diseases and supports a central role of microglial function in disease pathogenesis. Homozygous loss-of-function TREM2 variants cause Nasu-Hakola disease (Yamazaki, K. et al. (2015); Paloneva BM, J. et al. (2001); Ulrich J. D. et al. (2017)), whereas heterozygous loss-of-function TREM2 variants are associated with an increased risk for several neurological and neurodegenerative disorders such as Alzheimer's disease (AD), Frontotemporal lobar degeneration (FTLD), Parkinson's disease, FTLD-like syndrome, and Amyotrophic lateral sclerosis (ALS). The most prevalent mutation associated with AD is the loss-of-function mutation R47H, which has been shown to abrogate ligand binding and phagocytosis (Atagi, Y. et al. (2015); Kleinberger, G. et al (2014)).

Neurodegenerative disorders that may be treated by modulation of TREM2 activity and/or signaling include, but is not limited to, Alzheimer's disease (AD), Frontotemporal lobar degeneration (FTLD), FTLD-like syndrome, Parkinson's disease, Huntington disease, Nasu-Hakola disease (also known as polycystic lipomembranous osteodysplasia with sclerosing leukoencephalopathy), Multiple sclerosis (MS), Guillain-Barre syndrome, chronic inflammatory demyelinating polyneuropathies, Charcot-Marie-Tooth disease and Amyotrophic lateral sclerosis (ALS). Thus, there is a high and unmet medical need for TREM2 modulators to address these indications.

### SUMMARY

The present invention relates to compounds that modulates TREM2.

In one aspect, the present invention relates to a compound of Formula LXI:

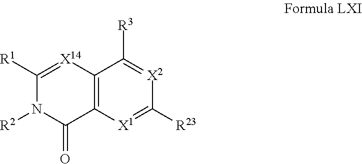

wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, C₅₋₈ bicycloalkyl, cyclopent-1-en-1-yl, cyclohex-1-en-1-yl, phenyl, 6-membered heteroaryl, 5-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl C₃₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl, C₁₋₃ haloalkyl and CN;
   wherein one methylene group of the C₃₋₆ cycloalkyl is optionally replaced with —O— or —N(R²⁸)—;
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XLVI:

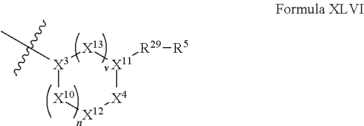

wherein
X³ is N or C(R⁸);
X⁴ is C(R³⁸)(R³⁹), O, NH, NR⁹ or a bond;
X¹⁰ is individually C(R³⁷)(R⁷) or C(O);
X¹¹ is C(R⁴) or N;
X¹² is C(R⁶)(R^{6b});
X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
   wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
   wherein when R⁶ and R^{6b} are C₁₋₃ alkyl, then R⁶ and R^{6b} are optionally linked together to form a 3-6 membered ring; and/or
   wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is individually H, C₁₋₃ alkyl or a bond;
   wherein when R⁷ is a bond then R⁶ is C₁₋₃ alkyl, and R⁷ and R⁶ are linked together to form a 3-5 membered ring;
R³⁷ is individually H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
R³⁸ is H or F;
R³⁹ is H or F;
R⁴⁰ is individually H, C₁₋₃ alkyl or a bond;
   wherein when R⁴⁰ is a bond then R⁸ is C₁₋₃ alkyl, and R⁸ and R⁴⁰ are linked together to form a 3-5 membered ring;
R⁴¹ is individually H or C₁₋₃ alkyl;
n is 0, 1 or 2; and
v is 0, 1 or 2,
or a pharmaceutically acceptable salt thereof.

In one aspect, the present invention relates a compound of Formula LXI as defined herein, or a pharmaceutically acceptable salt thereof, for use in the treatment of a disease associated with loss-of-function of TREM2, such as a neurodegenerative disease.

### Definitions

As used herein, the singular forms “a,” “an” and “the” include plural referents unless the content clearly dictates otherwise.

The terms “approximately” and “about” as referred herein are synonymous. In some embodiments, “about” refer to the recited amount, value, or duration ±20%, ±10%, ±5%, ±4%, ±3%, ±2%, ±1%, or ±0.5%.

The term, “compound,” as used herein is meant to include all stereoisomers, geometric isomers, tautomers, and isotopes of the structures depicted, unless otherwise specified.

The terms “C₁₋₃ alkyl”, “C₁₋₅ alkyl” and “C₁₋₆ alkyl” as used herein refer to a straight or branched hydrocarbon chains containing from 1 to 3, 1 to 5, and 1 to 6 carbon atoms, respectively. Representative examples of C₁₋₃ alkyl, C₁₋₅ alkyl and C₁₋₆ alkyl include, but are not limited to, methyl, ethyl, n-propyl, iso-propyl, n-butyl, sec-butyl, iso-butyl, tert-butyl, pentyl and hexyl.

The term “C₂₋₄ alkenyl” as used herein refers to a saturated hydrocarbon containing 2 to 4 carbon atoms having at least one carbon-carbon double bond. Alkenyl groups include both straight and branched moieties. Representative examples of C₂₋₄ alkenyl include, but are not limited to, 1-propenyl, 2-propenyl, 2-methyl-2-propenyl, and butenyl.

The term “C₃₋₆ cycloalkyl” as used herein refers to a saturated carbocyclic molecule wherein the cyclic framework has 3 to 6 carbon atoms. Representative examples of C₃₋₆ cycloalkyl include, but are not limited to, cyclopropyl, cyclobutyl, cyclopentyl, and cyclohexyl.

The terms “diC₁₋₃ alkylamino” as used herein refer to —NR*R**, wherein R* and R** independently represent a C₁₋₃ alkyl as defined herein. Representative examples of diC₁₋₃ alkylamino include, but are not limited to, —N(CH₃)₂, —N(CH₂CH₃)₂, —N(CH₃)(CH₂CH₃), —N(CH₂ CH₂CH₃)₂ and —N(CH(CH₃)₂)₂.

The term “C₁₋₃ alkoxy” and “C₁₋₆ alkoxy” as used herein refer to —OR^{#}, wherein R^{#} represents a C₁₋₃ alkyl and C₁₋₆ alkyl group, respectively, as defined herein. Representative examples of C₁₋₃ alkoxy and C₁₋₆ alkoxy include, but are not limited to, methoxy, ethoxy, propoxy, iso-propoxy, and butoxy.

The term “halogen” as used herein refers to —F, —Cl, —Br, or —I. In some embodiments, the halogen is F. In some embodiments, the halogen is Cl.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₆ haloalkyl” refers to a C₁₋₆ alkyl as defined herein, wherein one or more hydrogen atoms are substituted with a halogen.

Representative examples of C₁₋₆ haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃). Further, the term “C₁₋₆ haloalkoxy” for example refers to a C₁₋₆ alkoxy as defined herein, wherein one or more hydrogen atoms are substituted with a halogen. Representative examples of C₁₋₆ haloalkoxy include, but are not limited to, —OCH₂F, —OCHF₂, —OCF₃, —OCHFCl, —OCH₂CF₃, —OCFHCF₃, —OCF₂CF₃, —OCH(CF₃)₂, —OCF(CHF₂)₂, and —OCH(CH₂F)(CF₃).

The term “CN” is used herein to indicate a cyano group

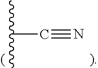 [SMILES: *C#N]

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆ heterocycloalkyl” as used herein refers to a saturated carbocyclic molecule wherein the cyclic framework has 3 to 6 carbons and wherein one or more carbon atoms are substituted with heteroatom(s) selected from N, O, and S. In some embodiments, a “C₃₋₆ heterocycloalkyl” refers to a saturated carbocyclic molecule wherein the cyclic framework has 3 to 6 carbons and wherein one carbon atom is substituted with a heteroatom selected from N, O, and S. If the C₃₋₆ heterocycloalkyl group is a C heterocycloalkyl, one or two carbon atoms are substituted with a heteroatom independently selected from N, O, and S. Representative examples of C₃₋₆ heterocycloalkyl include, but are not limited to, aziridinyl, azetidinyl, oxetanyl, pyrrolidinyl, piperazinyl, morpholinyl, and thiomorpholinyl.

The term “spiro compound” as used herein refers to a compound having one atom (usually a quaternary carbon) as the only common member of two rings, The term “C₅₋₈ spiroalkyl” as used herein refers to a bicyclic ring system comprising 5 to 8 carbon atoms, wherein the two rings are connected through a single common carbon atom. Representative examples of C₅₋₈ spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈ tricycloalkyl” as used herein refers a tricyclic ring system, wherein all three cycloalkyl rings share the same two ring atoms. Representative examples of C₅₋₈ tricycloalkyl include, but are not limited to, tricyclo[1.1.1.0^{1,3}]pentanyl, tricyclo[2.1.1.0^{1,4}]hexanyl, tricyclo[3.1.1.0^{1,5}]hexanyl and tricyclo[3.2.1.0^{1,5}]octanyl.

The term “C₅₋₈ bicycloalkyl” as used herein refers a bicyclic ring system, wherein both cycloalkyl rings share the same two ring atoms. The term “C₅₋₈ bicycloalkyl” includes bridged bicyclic compounds, i.e. wherein the two rings share three or more atoms, separating the two bridgehead atoms by a bridge containing at least one atom. For example, the term “C₅₋₈ bicycloalkyl” includes bicyclo[1.1.1]pentyl. For example, the term “C₅₋₈ bicycloalkyl” includes

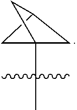 [SMILES: *C12CC(C1)C2]

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl” or “heteroaralkoxy” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As described herein, compounds of the present invention may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position, i.e. the substituent may be individually/independently selected from a group of substituents. Combinations of substituents envisioned by the present invention are preferably those that result in the formation of stable or chemically feasible compounds.

The term “stable,” as used herein, refers to compounds that are not substantially altered when subjected to conditions to allow for their production, detection, and, in certain embodiments, their recovery, purification, and use for one or more of the purposes disclosed herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art.

### DETAILED DESCRIPTION

In one aspect, the present invention relates to a compound of Formula LXI:

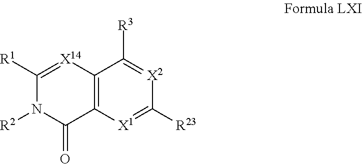

wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₃₋₈ spiroalkyl, C₅₋₈ tricycloalkyl, C₅₋₈ bicycloalkyl, cyclopent-1-en-1-yl, cyclohex-1-en-1-yl, phenyl, 6-membered heteroaryl, 5-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl, C₁₋₃ haloalkyl and CN;
   wherein one methylene group of the C₃₋₆ cycloalkyl is optionally replaced with —O— or —N(R²⁸)—;
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XLVI:

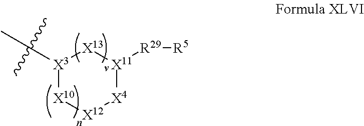

wherein
X³ is N or C(R⁸);
X⁴ is C(R³⁸)(R³⁹), O, NH, NR⁹ or a bond;
X¹⁰ is individually C(R³⁷)(R⁷) or C(O);
X¹¹ is C(R⁴) or N;
X¹² is C(R⁶)(R^{6b});
X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
   wherein when R⁶ and R^{6b} are C₁₋₃ alkyl, then R⁶ and R^{6b} are optionally linked together to form a 3-6 membered ring; and/or
   wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is individually H, C₁₋₃ alkyl or a bond;
   wherein when R⁷ is a bond then R⁶ is C₁₋₃ alkyl, and R⁷ and R⁶ are linked together to form a 3-5 membered ring;
R³⁷ is individually H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
R³⁸ is H or F;
R³⁹ is H or F;
R⁴⁰ is individually H, C₁₋₃ alkyl or a bond;
   wherein when R⁴⁰ is a bond then R⁸ is C₁₋₃ alkyl, and R⁸ and R⁴⁰ are linked together to form a 3-5 membered ring;
R⁴¹ is individually H or C₁₋₃ alkyl;
n is 0, 1 or 2; and
v is 0, 1 or 2,
or a pharmaceutically acceptable salt thereof.

In one aspect, the present invention relates to a compound of Formula LXI:

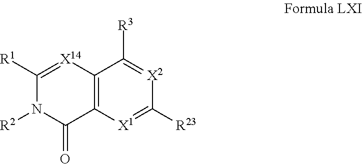

wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₈ cycloalkyl, C₅₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl and C₁₋₃ haloalkyl;
   wherein one methylene group of the C₃₋₆ cycloalkyl is optionally replaced with —O— or —N(R²⁸);
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XLVI:

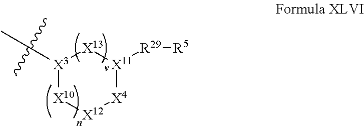

wherein
X³ is N or C(R⁸);
X⁴ is C(R³⁸)(R³⁹), O, NH, NR⁹ or a bond;
X¹⁰ is individually C(R³⁷)(R⁷) or C(O);
X¹¹ is C(R⁴) or N;
X¹² is C(R⁶)(R^{6b});
X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
   wherein when R⁶ and R^{6b} are C₁₋₃ alkyl, then R⁶ and R^{6b} are optionally linked together to form a 3-6 membered ring; and/or
   wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is individually H or C₁₋₃ alkyl;
R³⁷ is individually H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
R³⁸ is H or F;
R³⁹ is H or F;
R⁴⁰ is individually H, C₁₋₃ alkyl or a bond;
   wherein when R⁸ is a bond and R⁴⁰ C₁₋₃ alkyl, then R⁸ and R⁴⁰ are optionally linked together to form a 3-5 membered ring;
R⁴¹ is individually H or C₁₋₃ alkyl;
n is 0, 1 or 2; and
v is 0, 1 or 2,
or a pharmaceutically acceptable salt thereof.

In one aspect, the present invention relates to a compound of Formula LXI:

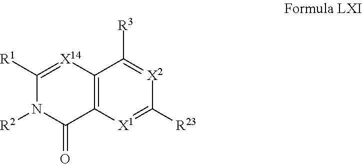

wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl and C₁₋₃ haloalkyl;
   wherein one methylene group of the C₃₋₆ cycloalkyl is optionally replaced with —O— or —N(R²⁸)—;
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XLVI:

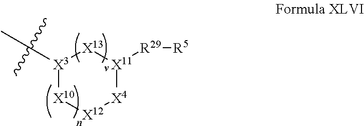

wherein
X³ is N or C(R⁸);
X⁴ is C(R³⁸)(R³⁹), O, NH, NR⁹ or a bond;
X¹⁰ is individually C(R³⁷)(R⁷) or C(O); X¹¹ is C(R⁴) or N;
X¹² is C(R⁶)(R^{6b}); X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
   wherein when R⁶ and R^{6b} are C₁₋₃ alkyl, then R⁶ and R^{6b} are optionally linked together to form a 3-6 membered ring; and/or
   wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl; R⁷ is individually H or C₁₋₃ alkyl; R³⁷ is individually H or C₁₋₃ alkyl; R⁸ is selected from the group consisting of H, —OH, —O-alkyl and halogen; R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen; R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl; R³⁸ is H or F; R³⁹ is H or F; R⁴⁰ is individually H, C₁₋₃ alkyl or a bond;
wherein when R⁸ is a bond and R⁴⁰ C₁₋₃ alkyl, then R⁸ and R⁴⁰ are optionally linked together to form a 3-5 membered ring; R⁴¹ is individually H or C₁₋₃ alkyl;
n is 0, 1 or 2; and
v is 1 or 2,
or a pharmaceutically acceptable salt thereof.

In one aspect, the present invention concerns a compound of Formula IX:

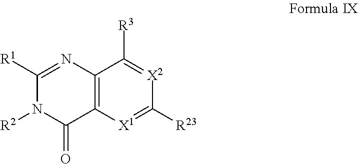

wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₈ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl and C₁₋₃ haloalkyl;
   wherein one methylene group of the C₃₋₆ cycloalkyl is optionally replaced with —O— or —N(R²⁸)—;
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XX:

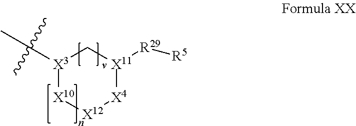

wherein
X³ is N or C(R⁸);
X⁴ is CH₂, O, CHF, CF₂, NH, NR⁹ or a bond;
X¹⁰ is C(H)(R⁷) or C(O);
X¹¹ is C(R⁴) or N;
X¹² is C(R⁶)(R^{6b});
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
   wherein when R⁶ and R^{6b} are C₁₋₃ alkyl, then R⁶ and R^{6b} are optionally linked together to form a 3-6 membered ring; and/or
   wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
n is 0, 1 or 2; and
v is 1 or 2
or a pharmaceutically acceptable salt thereof.

In one aspect, the present invention concerns a compound of Formula IX:

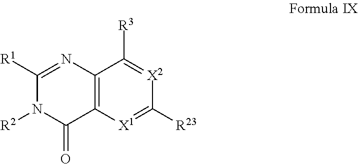

wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
   wherein one methylene group of the C₃₋₆ cycloalkyl is optionally replaced with —O— or —N(R²⁸)—;
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is selected from the group consisting of —O—C₁₋₆ alkyl and C₃₋₆ cycloalkyl;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XX:

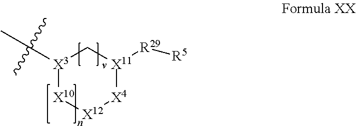

wherein
X³ is N or C(R⁸);
X⁴ is CH₂, O, CHF, CF₂, NH, NR⁹ or a bond;
X¹⁰ is C(H)(R⁷) or C(O);
X¹¹ is C(R⁴) or N;
X¹² is C(R⁶)(R^{6b});
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
   wherein when R⁶ and R^{6b} are C₁₋₃ alkyl, then R⁶ and R^{6b} are optionally linked together to form a 3-6 membered ring; and/or
   wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
n is 0, 1 or 2; and
v is 1 or 2,
or a pharmaceutically acceptable salt thereof.

In one aspect, the present invention relates to a compound of Formula IX:

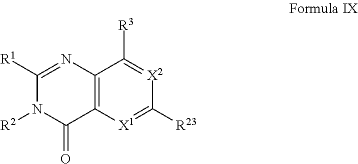

wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
   wherein one methylene group of the C₃₋₆ cycloalkyl is optionally replaced with —O— or —N(R²⁸)—;
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is selected from the group consisting of —O—C₁₋₆ alkyl and C₃₋₆ cycloalkyl;
R¹¹ is selected from the group consisting of —O—C₁₋₆ alkyl, C₃₋₆ cycloalkyl and C₁₋₆ haloalkyl;
R²³ is of Formula XVI:

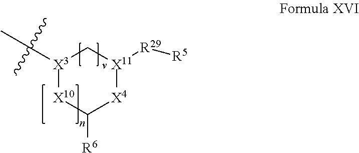

wherein
X³ is N or C(R⁸);
X⁴ is CH₂, O, CHF, CF₂, NH, NR⁹ or a bond;
X¹⁰ is C(H)(R⁷) or C(O);
X¹¹ is C(R⁴) or N;
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H or C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
n is 0, 1 or 2; and
v is 1 or 2
or a pharmaceutically acceptable salt thereof.

In one aspect, the present invention relates to a compound of Formula IX:

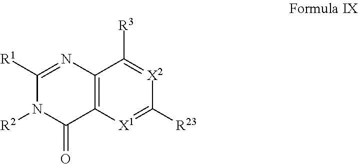

wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₈ cycloalkyl is optionally substituted with R¹⁰; R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
   wherein one methylene group of the C₃₋₆ cycloalkyl is optionally replaced with —O— or —N(R²⁸);
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is selected from the group consisting of —O—C₁₋₆ alkyl and C₃₋₆ cycloalkyl;
R¹¹ is selected from the group consisting of —O—C₁₋₆ alkyl, C₃₋₆ cycloalkyl and C₁₋₆ haloalkyl;
R²³ is of Formula X:

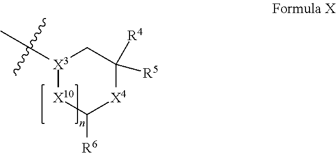

wherein
X³ is N or C(R³);
X⁴ is CH₂, O, CHF, CF₂, NH, NR⁹ or a bond;
X¹⁰ is C(H)(R⁷) or C(O);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with oxo (═O), and
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with —N(H)C(O)R²⁴, -oxo (═O), —O—C₁₋₃ alkyl and —N(R³⁰)(R³¹);
R⁶ is H or C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl or —C(O)—C₃₋₆ cycloalkyl;
R²⁴ is C₁₋₆ alkyl or aryl, wherein the C₁₋₆ alkyl is optionally substituted with C₁₋₃ alkoxy or halogen; and
n is 0, 1 or 2,
or a pharmaceutically acceptable salt thereof.

In some embodiments, the compound is of Formula I:

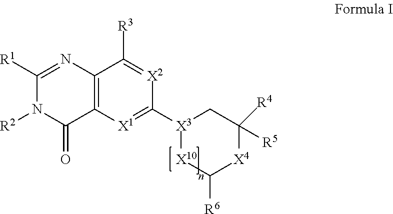

wherein
X¹ is N or CH;
X² is N or CH;
X³ is N or C(R⁸);
X⁴ is CH₂, O, CHF, CF₂, NH, NR⁹ or a bond;
X¹⁰ is C(H)(R⁷) or C(O);
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl,
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, 5-membered heteroaryl and 6-membered heteroaryl, wherein
   (a) the C₃₋₆ cycloalkyl or C₃₋₆ heterocycloalkyl is optionally substituted with oxo (═O), and
   (b) the phenyl, 5-membered heteroaryl or 6-membered heteroaryl is optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃-6 cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
R⁶ is H or C₁₋₃ alkyl;
R⁷ is H or C₁₋₃ alkyl;
R⁸ is selected from the group consisting of H, —OH, —O-alkyl and halogen;
   R⁹ is C₁₋₆ alkyl, —C(O)—C₃₋₆ cycloalkyl, —C(O)—C₁₋₆ alkyl, C₁₋₆ alkyl-O—C₁₋₆ alkyl or C₁₋₆ haloalkyl;
   R¹⁰ is selected from the group consisting of —O—C₁₋₆ alkyl and C₃₋₆ cycloalkyl;
   R¹¹ is selected from the group consisting of —O—C₁₋₆ alkyl and C₃₋₆ cycloalkyl;
n is 0, 1 or 2,
or a pharmaceutically acceptable salt thereof.

In some embodiments, R²³ is of Formula XLVII, wherein R⁵, R⁸, R²⁹, X⁴, X¹⁰, X¹¹, X¹², n and v are as defined herein. In some embodiments, R²³ is of Formula XLVIII, wherein R⁵, R⁸, R²⁹, X⁴, X¹⁰, X¹¹, X¹², n and v are as defined herein. In some embodiments, R²³ 5 is of Formula XLIX, wherein R⁴, R⁵, R²⁹, X⁴, X¹⁰, X¹², X¹³, n and v are as defined herein.

In some embodiments, R²³ is of Formula L, wherein R⁴, R⁵, R²⁹, X⁴, X¹⁰, X¹², X¹³, n and v are as defined herein. In some embodiments, R²³ is of Formula LI, wherein R⁴, R⁵, R⁸, R²⁹, X⁴, X¹⁰, X¹², X¹³, n and v are as defined herein. In some embodiments, R²³ is of Formula LII, wherein R⁴, R⁵, R⁸, R²⁹, X⁴, X¹⁰, X¹², X¹³, n and v are as defined herein.

In some embodiments, R²³ is of Formula LIII, wherein R⁴, R⁵, R⁸, R²⁹, X⁴, X¹⁰, X¹², X¹³, n and v are as defined herein. In some embodiments, R²³ is of Formula LIV, wherein R⁴, R⁵, R⁸, R²⁹, X⁴, X¹⁰, X¹², X¹³, n and v are as defined herein.

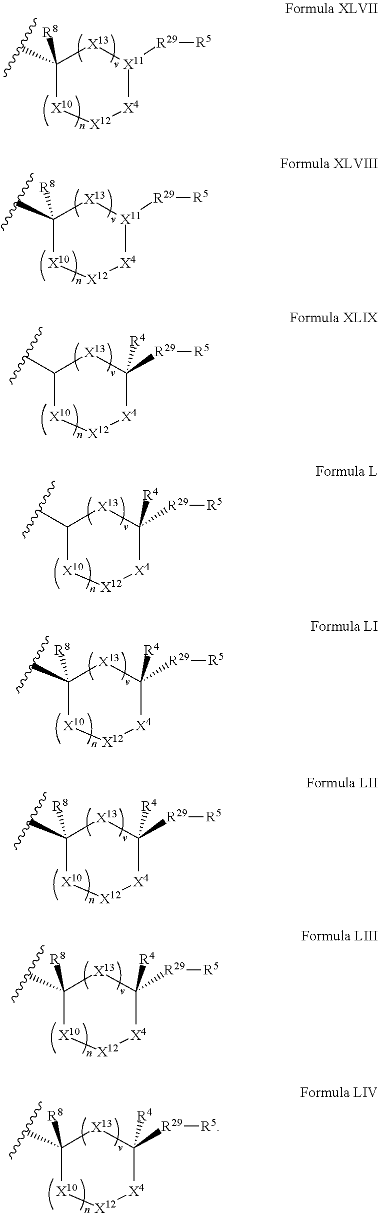

In some embodiments, R²³ is of Formula Xa, wherein R⁴, R⁵, R⁶, X³, X⁴, X¹⁰ and n are as defined herein, or a pharmaceutically acceptable salt thereof. In some embodiments, R²³ is of Formula Xb, wherein R⁴, R⁵, R⁶, X³, X⁴, X¹⁰ and n are as defined herein, or a pharmaceutically acceptable salt thereof. In some embodiments, R²³ is of Formula XXa,
wherein R⁴, R⁵, R²⁹, X³, X⁴, X¹⁰, X¹², n and v are as defined herein, or a pharmaceutically acceptable salt thereof. In some embodiments, R²³ is of Formula XXb,
wherein R⁴, R⁵, R²⁹, X³, X⁴, X¹⁰, X¹², n and v are as defined herein, or a pharmaceutically acceptable salt thereof. In some embodiments, R²³ is of Formula XXc,
wherein R⁴, R⁵, R⁸, R²⁹, X⁴, X¹⁰, X¹², n and v are as defined herein, or a pharmaceutically acceptable salt thereof. In some embodiments, R²³ is of Formula XXd,
wherein R⁴, R⁵, R⁸, R²⁹, X⁴, X¹⁰, X¹², n and v are as defined herein, or a pharmaceutically acceptable salt thereof. In some embodiments, R²³ is of Formula XXe,
wherein R⁴, R⁵, R⁸, R²⁹, X⁴, X¹⁰, X¹², n and v are as defined herein, or a pharmaceutically acceptable salt thereof. In some embodiments, R²³ is of Formula XXf,
wherein R⁴, R⁵, R⁸, R²⁹, X⁴, X¹⁰, X¹², n and v are as defined herein, or a pharmaceutically acceptable salt thereof.

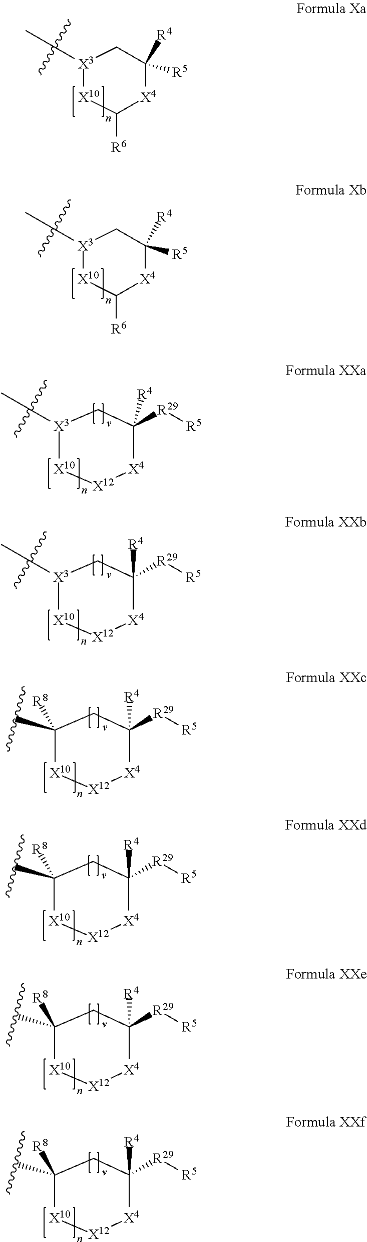

In some embodiments, the compound is of Formula Ia or Formula Ib:

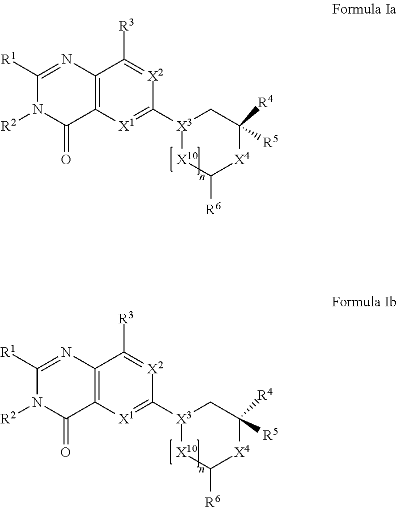

wherein R¹, R², R³, R⁴, R⁵, R⁶, X³, X⁴, X¹⁰ and n are as defined herein, or a pharmaceutically acceptable salt thereof.

In some embodiments, X¹ is N. In some embodiments, X¹ is C(R⁴²), wherein R⁴² is H or halogen. In some embodiments, R⁴² is H, i.e. X¹ is CH. In some embodiments, R⁴² is halogen. In some embodiments, R⁴² is F, i.e. X¹ is CF. In some embodiments, X¹ is CH. In some embodiments, X² is N. In some embodiments, X² is CH. In some embodiments, X¹⁴ is N. In some embodiments, X¹⁴ is CH. In some embodiments, X¹ is N and X² is CH. In some embodiments, X¹ is CH and X² is N. In some embodiments, X¹ is CH and X² is CH. In some embodiments, X¹ is N and X² is N. In some embodiments, X¹ is N, X² is N and X¹⁴ is N. In some embodiments, X¹ is N, X² is N and X¹⁴ is CH. In some embodiments, X¹ is N, X² is CH and X¹⁴ is N. In some embodiments, X¹ is CH, X² is N and X¹⁴ is N. In some embodiments, X¹ is CH, X² is CH and X¹⁴ is N. In some embodiments, X¹ is CH, X² is N and X¹⁴ is CH. In some embodiments, X¹ is N, X² is CH and X¹⁴ is CH. In some embodiments, X¹ is CH, X² is CH and X¹⁴ is CH. In some embodiments, X¹ is CF, X² is N and X¹⁴ is N.

In some embodiments, the compound is of Formula II:

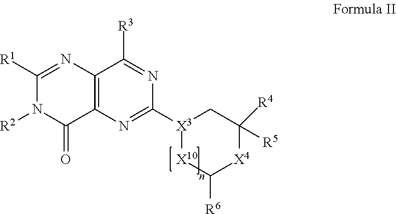

wherein R¹, R², R³, R⁴, R⁵, R⁶, X¹, X⁴, X and n are as defined herein, or a pharmaceutically acceptable salt thereof.

In some embodiments, the compound is of Formula II, wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
   wherein one methylene group of the C₃₋₆ cycloalkyl is optionally replaced with —O— or —N(R²⁸)—;
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
   R¹⁰ is selected from the group consisting of —O—C₁₋₆ alkyl and C₃₋₆ cycloalkyl;
R¹¹ is selected from the group consisting of —O—C₁₋₆ alkyl, C₃₋₆ cycloalkyl and C₁₋₆ haloalkyl;
X³ is N or C(R⁸);
X⁴ is CH₂, O, CHF, CF₂, NH, NR⁹ or a bond;
X¹⁰ is CH₂;
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with oxo (═O), and
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with —N(H)C(O)R²⁴, -oxo (═O), —O—C₁₋₃ alkyl and —N(R³⁰)(R³¹);
R⁶ is H or C₁₋₃ alkyl;
R⁸ is selected from the group consisting of H, —OH, —O-alkyl and halogen;
R⁹ is C₁₋₆ alkyl, —C(O)—C₁₋₆ alkyl or —C(O)—C₃₋₆ cycloalkyl;
R²⁴ is C₁₋₆ alkyl or aryl, wherein the C₁₋₆ alkyl is optionally substituted with C₁₋₃ alkoxy or halogen; and
n is 1,
or a pharmaceutically acceptable salt thereof.

In some embodiments, R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰; R^{a} is H or C₁₋₆ alkyl; R^{b} is H or C₁₋₆ alkyl; and R¹⁰ is selected from the group consisting of —O—C₁₋₆ alkyl, C₃₋₆ cycloalkyl and halogen. In some embodiments, R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰; R^{a} is H or C₁₋₆ alkyl; R^{b} is H or C₁₋₆ alkyl; and R¹⁰ is selected from the group consisting of —O—C₁₋₆ alkyl, C₃₋₆ cycloalkyl and halogen. In some embodiments, R¹ is C₁₋₆ alkyl optionally substituted with 1 to 3 individually selected substituents R¹⁰. In some embodiments, R¹ is C₁₋₆ alkyl optionally substituted with R¹⁰ as defined herein. In some embodiments, R¹ is C₁₋₃ alkyl optionally substituted with 1 to 3 individually selected substituents R¹⁰. In some embodiments, R¹ is C₁₋₃ alkyl optionally substituted with R¹⁰ as defined herein. In some embodiments, R¹ is —CH₃. In some embodiments, R¹⁰ is individually selected from the group consisting of —O—C₁₋₆ alkyl, C₃₋₆ cycloalkyl and halogen. In some embodiments, R¹⁰ is halogen, such as F. In some embodiments, R¹ is —CF₃. In some embodiments, R¹ is C₃₋₆ cycloalkyl optionally substituted with R¹⁰ as defined herein. In some embodiments, R¹ is H.

In some embodiments, R² is selected from the group consisting of C₁₋₆ alkyl, C₃₋₆ cycloalkyl and H, wherein the C₁₋₆ alkyl or C₃₋₆ cycloalkyl is optionally substituted with R¹¹, wherein R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl. In some embodiments, R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl. In some embodiments, R¹¹ is selected from the group consisting of —O—C₁₋₆ alkyl, C₃₋₆ cycloalkyl and C₁₋₆ haloalkyl. In some embodiments, R² is selected from the group consisting of C₁₋₆ alkyl, C₃₋₆ cycloalkyl and H, wherein the C₁₋₆ alkyl or C₃₋₆ cycloalkyl is optionally substituted with R¹¹, wherein R¹¹ is selected from the group consisting of —O—C₁₋₆ alkyl, C₃₋₆ cycloalkyl and C₁₋₆ haloalkyl. In some embodiments, R² is C₁₋₆ alkyl optionally substituted with R¹¹ as defined herein. In some embodiments, R² is C₁₋₃ alkyl optionally substituted with R¹¹ as defined herein. In some embodiments, R² is —CH₃. In some embodiments, R² is C₃₋₆ cycloalkyl optionally substituted with R¹¹ as defined herein. In some embodiments, R¹¹ is —O—C₁₋₆ alkyl, such as —O—CH₃. In some embodiments, R² is H. In some embodiments, R² is C₁₋₃ alkyl substituted with R¹¹, and R¹¹ is —O—C₁₋₆ alkyl. In some embodiments, R² is —CH₂CH₂OCH₃. In some embodiments, R² is C₁₋₆ alkyl substituted with phenyl, wherein the phenyl is optionally substituted with —O—C₁₋₆ alkyl. In some embodiments, R² is C₁ alkyl substituted with phenyl, wherein the phenyl is substituted with —O—C₁₋₆ alkyl, such as —O—C₁₋₃ alkyl, for example —O—C₁ alkyl.

In some embodiments, R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl, C₁₋₃ haloalkyl and CN;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy.

In some embodiments, R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, —CN, C₁₋₃ alkyl and C₁₋₃ haloalkyl;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy.

In some embodiments, R³ is phenyl optionally substituted with 1 to 4 substituents independently selected from the group consisting of halogen, C₁₋₃ alkyl, C₁₋₃ haloalkyl and CN, or a pharmaceutically acceptable salt thereof.

In some embodiments, R³ is phenyl optionally substituted with 1 to 4 substituents independently selected from the group consisting of halogen, —CN, C₁₋₃ alkyl and C₁₋₃ haloalkyl.

In some embodiments, R³ is of Formula III:

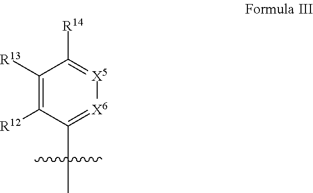

wherein
X⁵ is C(R¹⁵) or N;
X⁶ is CH or N;
R¹² is H, halogen, C₁₋₃ alkyl, C₁₋₃ haloalkyl or CN;
R¹³ is H, halogen, C₁₋₃ alkyl, C₁₋₃ haloalkyl or CN;
R¹⁴ is H, halogen, C₁₋₃ alkyl, C₁₋₃ haloalkyl or CN; and
R¹⁵ is H, halogen, C₁₋₃ alkyl, C₁₋₃ haloalkyl or CN,
or a pharmaceutically acceptable salt thereof.

In some embodiments, R³ is of Formula III, wherein X⁵ is C(R¹⁵) or N; X⁶ is CH or N; R¹² is H, halogen, C₁₋₃ alkyl or C₁₋₃ haloalkyl; R¹³ is H, halogen, C₁₋₃ alkyl or C₁₋₃ haloalkyl; R¹⁴ is H, halogen, C₁₋₃ alkyl or C₁₋₃ haloalkyl; and R¹⁵ is H, halogen, C₁₋₃ alkyl or C₁₋₃ haloalkyl.

In some embodiments, R³ is of Formula III, wherein X⁵ is C(R¹⁵) or N; X⁶ is CH; R¹² is H or halogen; R¹³ is H or halogen; R¹⁴ is H, halogen, or C₁₋₃ haloalkyl; and R¹⁵ is H or halogen, or a pharmaceutically acceptable salt thereof.

In some embodiments, X⁵ is C(R¹⁵) and X⁶ is CH. Thus, in some embodiments, R³ is of Formula IV:

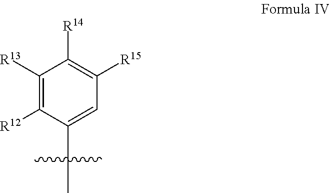

wherein
R¹² is H, halogen, C₁₋₃ alkyl, C₁₋₃ haloalkyl or CN;
R¹³ is H, halogen, C₁₋₃ alkyl, C₁₋₃ haloalkyl or CN;
R¹⁴ is H, halogen, C₁₋₃ alkyl, C₁₋₃ haloalkyl or CN; and
R¹⁵ is H, halogen, C₁₋₃ alkyl, C₁₋₃ haloalkyl or CN,
or a pharmaceutically acceptable salt thereof.

in some embodiments, R³ is of Formula IV, wherein R¹² is H, halogen, C₁₋₃ alkyl or C₁₋₃ haloalkyl; R¹³ is H, halogen, C₁₋₃ alkyl or C₁₋₃ haloalkyl; R¹⁴ is H, halogen, C₁₋₃ alkyl or C₁₋₃ haloalkyl; and R¹⁵ is H, halogen, C₁₋₃ alkyl or C₁₋₃ haloalkyl.

In some embodiments, X⁵ is N and X⁶ is CH. In some embodiments, X⁵ is CH and X⁶ is N.

In some embodiments, R¹² is H. In some embodiments, R¹² is halogen, such as F or C.

In some embodiments, R¹² is C₁₋₃ alkyl, such as —CH₃. In some embodiments, R¹² is CN.

In some embodiments, R¹³ is H. In some embodiments, R¹³ is halogen, such as F.

In some embodiments, R¹⁴ is halogen, such as F or Cl. In some embodiments, R¹⁴ is C₁₋₃ alkyl, such as —CH₃. In some embodiments, R¹⁴ is C₁₋₃ haloalkyl. In some embodiments, R¹⁴ is —CF₃. In some embodiment, R¹⁴ is —CHF₂. In some embodiments, R¹⁴ is CN.

In some embodiments, R¹⁵ is H. In some embodiments, R¹⁵ is halogen, such as F.

In some embodiments, R³ is

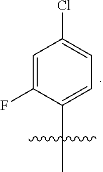 [SMILES: *c1ccc(Cl)cc1F]

In some embodiments, R³ is

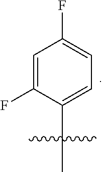 [SMILES: *c1ccc(F)cc1F]

In some embodiments, R³ is

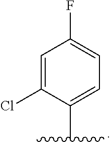 [SMILES: *c1ccc(F)cc1Cl]

In some embodiments, R³ is

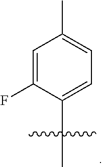 [SMILES: *c1ccc(C)cc1F]

In some embodiments, R³ is

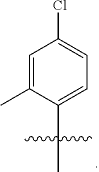 [SMILES: *c1ccc(Cl)cc1C]

In some embodiments, R³ is

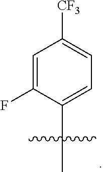 [SMILES: *c1ccc(C(F)(F)F)cc1F]

in some embodiments, R³ is

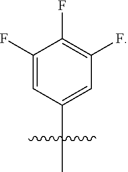 [SMILES: *c1cc(F)c(F)c(F)c1]

In some embodiments, R³ is

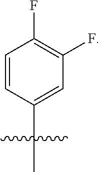 [SMILES: *c1ccc(F)c(F)c1]

In some embodiments, R³ is

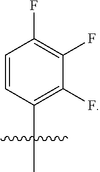 [SMILES: *c1ccc(F)c(F)c1F]

In some embodiments, R³ is

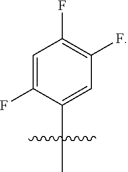 [SMILES: *c1cc(F)c(F)cc1F]

In some embodiments, R³ is

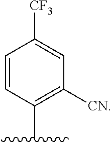 [SMILES: *c1ccc(C(F)(F)F)cc1C#N]

In some embodiments, R³ is

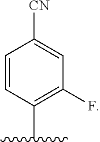 [SMILES: *c1ccc(C#N)cc1F]

In some embodiments, R³ is

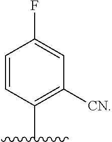 [SMILES: *c1ccc(F)cc1C#N]

In some embodiment, R³ is

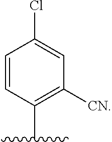 [SMILES: *c1ccc(Cl)cc1C#N]

In some embodiments, R³ is

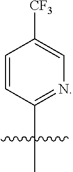 [SMILES: *c1ccc(C(F)(F)F)cn1]

In some embodiments, R³ is

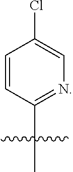 [SMILES: *c1ccc(Cl)cn1]

In some embodiments, R³ is

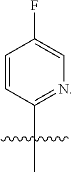 [SMILES: *c1ccc(F)cn1]

In some embodiments, R³ is

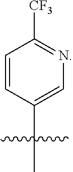 [SMILES: *c1ccc(C(F)(F)F)nc1]

In some embodiments, R³ is

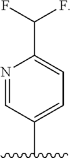 [SMILES: *c1ccc(C(F)F)nc1]

In some embodiments, R³ is a 5-membered heteroaryl optionally substituted with 1 to 4 substituents independently selected from the group consisting of halogen, C₁₋₃ alkyl and C₁₋₃ haloalkyl. In some embodiments, R³ is a 5-membered heteroaryl optionally substituted with 1 to 3 substituents independently selected from the group consisting of halogen, C₁₋₃ alkyl and C₁₋₃ haloalkyl. In some embodiments, R³ is a pyrazolyl optionally substituted with 1 to 3 substituents independently selected from the group consisting of halogen, C₁₋₃ alkyl and C₁₋₃ haloalkyl. In some embodiments, R³ is

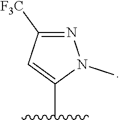 [SMILES: *c1cc(C(F)(F)F)nn1C]

In some embodiments, R³ is C₃₋₆ cycloalkyl optionally substituted with 1 to 4 substituents independently selected from the group consisting of halogen, C₁₋₃ alkyl and C₁₋₃ haloalkyl.

In some embodiments, R³ is of Formula V:

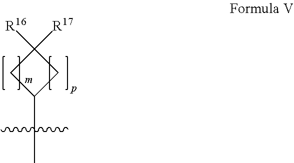

wherein
R¹⁶ is H, halogen, C₁₋₃ alkyl and C₁₋₃ haloalkyl;
R¹⁷ is H, halogen, C₁₋₃ alkyl and C₁₋₃ haloalkyl;
m is 1, 2 or 3; and
p is 1, 2 or 3.

In some embodiments, m is 1. In some embodiments, m is 2. In some embodiments, p is 1. In some embodiments, p is 2. In some embodiments, R¹⁶ is H. In some embodiments, R¹⁶ is halogen, such as F. In some embodiments, R¹⁶ is C₁₋₃ alkyl, such as —CH₃. In some embodiments, R¹⁶ is C₁₋₃ haloalkyl, such as —CF₃ or —CHF₂. In some embodiments, R¹⁷ is H. In some embodiments, R¹⁷ is halogen, such as F. In some embodiments, R¹⁷ is C₁₋₃ alkyl, such as —CH₃. In some embodiments, R¹⁷ is C₁₋₃ haloalkyl, such as —CF₃ or —CHF₂. In some embodiments, R³ is

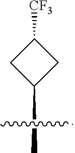

In some embodiments, R³ is

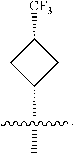

In some embodiments, R³ is

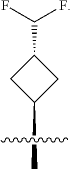

In some embodiments, R³ is

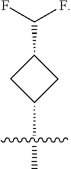

In some embodiments, R³ is

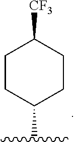 [SMILES: *[C@H]1CC[C@H](C(F)(F)F)CC1]

In some embodiments, R³ is

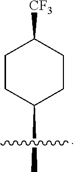

In some embodiments, R³ is

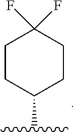

In some embodiments, R³ is

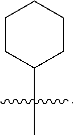 [SMILES: *C1CCCCC1]

In some embodiments, R³ is

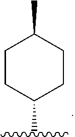 [SMILES: *[C@H]1CC[C@H](C)CC1]

In some embodiments, R³ is

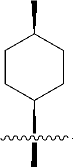

In some embodiments, R³ is

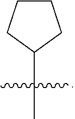 [SMILES: *C1CCCC1]

In some embodiments, R³ is

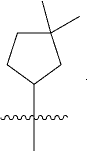 [SMILES: *C1CCC(C)(C)C1]

In some embodiments, R³ is C₁₋₃ haloalkyl, such as —(CH₂)₂CF₃.

In some embodiments, R³ is C₅₋₈ spiroalkyl optionally substituted with 1 to 4 substituents independently selected from the group consisting of halogen, C₁₋₃ alkyl and C₁₋₃ haloalkyl. In some embodiments, R³ is

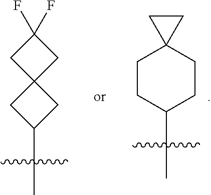 [SMILES: *C1CC2(C1)CC(F)(F)C2 ; *C1CCC2(CC1)CC2]

In some embodiments, R³ is C₅₋₈ tricycloalkyl optionally substituted with 1 to 4 substituents independently selected from the group consisting of halogen, C₁₋₃ alkyl and C₁₋₃ haloalkyl. In some embodiments, R³ is C₅₋₈ bicycloalkyl optionally substituted with 1 to 4 substituents independently selected from the group consisting of halogen, C₁₋₃ alkyl and C₁₋₃ haloalkyl. In some embodiment, R³ is bicyclo[1.1.1]pentyl optionally substituted with CF₃ or C₁ alkyl, or a pharmaceutically acceptable salt thereof In some embodiments, R³ is

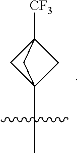 [SMILES: *C12CC(C(F)(F)F)(C1)C2]

In some embodiments, R³ is of Formula VI:

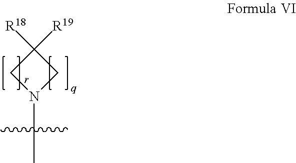

wherein
R¹⁸ is H, halogen, C₁₋₃ alkyl and C₁₋₃ haloalkyl;
R¹⁹ is H, halogen, C₁₋₃ alkyl and C₁₋₃ haloalkyl;
q is 1, 2 or 3; and
r is 1, 2 or 3.

In some embodiments, q is 1. In some embodiments, q is 2. In some embodiments, r is 1. In some embodiments, r is 2.

In some embodiments, R¹⁸ is H. In some embodiments, R¹⁸ is halogen, such as F. In some embodiments, R¹⁸ is C₁₋₃ alkyl, such as —CH₃. In some embodiments, R¹⁸ is C₁₋₃ haloalkyl, such as —CF₃ or —CHF₂.

In some embodiments, R¹⁹ is H. In some embodiments, R¹⁹ is halogen, such as F. In some embodiments, R¹⁹ is C₁₋₃ alkyl, such as —CH₃. In some embodiments, R¹⁹ is C₁₋₃ haloalkyl, such as —CF₃ or —CHF₂.

In some embodiments, R³ is

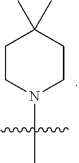 [SMILES: *N1CCC(C)(C)CC1]

In some embodiments, R³ is

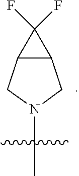 [SMILES: *N1CC2C(C1)C2(F)F]

In some embodiments, R³ is

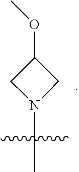 [SMILES: *N1CC(OC)C1]

In some embodiments, R³ is

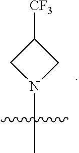 [SMILES: *N1CC(C(F)(F)F)C1]

In some embodiments, R³ is

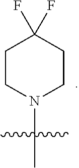 [SMILES: *N1CCC(F)(F)CC1]

In some embodiments, R³ is

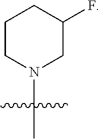 [SMILES: *N1CCCC(F)C1]

In some embodiments, R³ is —OCH₂—(C₃₋₆ cycloalkyl) optionally substituted with 1 to 4 substituents independently selected from the group consisting of halogen, C₁₋₃ alkyl, C₁₋₃ haloalkyl, C₁₋₃ alkoxy and C₁₋₃ haloalkoxy.

In some embodiments, R³ is

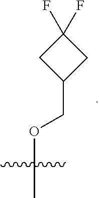 [SMILES: *OCC1CC(F)(F)C1]

In some embodiments, R³ is C₃₋₆ cycloalkyl wherein one or more methylene group is replaced with —O—. In some embodiments, R³ is

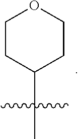 [SMILES: *C1CCOCC1]

In some embodiments, R³ is C₃₋₆ cycloalkyl wherein one or more methylene group is replaced with —N(R²⁸)—, wherein R²⁸ is selected from the group consisting of H, C₁₋₃ alkyl and —C(O)C₃ alkyl. In some embodiments, R³ is of Formula XV:

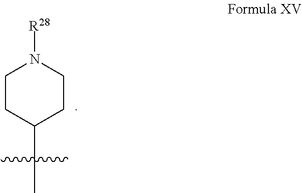

wherein R²⁸ is selected from the group consisting of H, C₁₋₃ alkyl and —C(O)C₁₋₃ alkyl, or a pharmaceutically acceptable salt thereof.

In some embodiments, R³ is selected from the group consisting of:

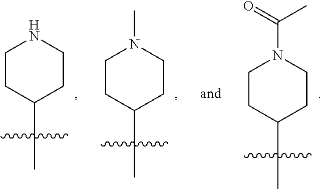

In some embodiments, R²³ is of Formula XLVI:

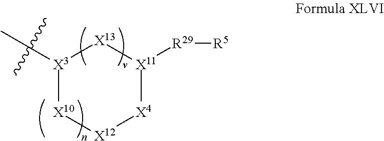

wherein
X³ is N or C(R⁸);
X⁴ is C(R³⁸)(R³⁹), O, NH, NR⁹ or a bond;
X¹⁰ is individually C(R³⁷)(R⁷) or C(O);
X¹¹ is C(R⁴) or N;
X¹² is C(Re)(R^{6b});
X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
      wherein the C₁₋₆ alkyl or C₁₋₆ haloalkyl of subsection (b) are optionally substituted with —OH, and
      wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
   (c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
   wherein when R⁶ and R^{6b} are C₁₋₃ alkyl, then R⁶ and R^{6b} are optionally linked together to form a 3-6 membered ring; and/or
   wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is individually H, C₁₋₃ alkyl or a bond;
   wherein when R⁷ is a bond then R⁶ is C₁₋₃ alkyl, and R⁷ and R⁶ are linked together to form a 3-5 membered ring;
R³⁷ is individually H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁-6 alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
R³⁸ is H or F;
R³⁹ is H or F;
R⁴⁰ is individually H, C₁₋₃ alkyl or a bond;
   wherein when R⁴⁰ is a bond then R⁸ is C₁₋₃ alkyl, and R⁸ and R⁴⁰ are linked together to form a 3-5 membered ring;
R⁴¹ is individually H or C₁₋₃ alkyl;
n is 0, 1 or 2; and
v is 0, 1 or 2.

In some embodiments, R²³ is a 6 membered ring, i.e. X³, (X¹³)ᵥ, X¹¹, X⁴, X¹² and (X¹⁰)ₙ together form a 6 membered ring. For example, in some embodiments, R²³ is of Formula XLVI wherein n is 1, v is 1 and is not a bond. In some embodiments, R²³ is of Formula XLVI wherein X³ is N; X⁴ is O; X¹⁰ is C(R³⁷)(R⁷); X¹¹ is C(R⁴); X¹² is C(R⁸)(R^{8b}); X¹³ is C(R⁴⁰)(R⁴¹); n is 1; and v is 1. In some embodiments, R²³ is of Formula XLVI wherein X³ is N; X⁴ is NH or NR⁹; X¹⁰ is C(R³⁷)(R⁷); X¹¹ is C(R⁴); X¹² is C(R⁶)(R^{6b}), X¹³ is C(R⁴⁰)(R⁴¹); n is 1; and v is 1. In some embodiments, R²³ is of Formula XLVI wherein X³ is N; X⁴ is C(R³⁸)(R³⁹); X¹⁰ is C(R³⁷)(R⁷); X¹¹ is C(R⁴); X¹² is C(R⁸)(R^{8b}); X¹³ is C(R⁴⁰)(R⁴¹); n is 1; and v is 1. In some embodiments, R²³ is of Formula XLVI wherein X³ is N; X⁴ is a bond; X¹⁰ is C(R³⁷)(R⁷); X¹¹ is C(R⁴); X¹² is C(R⁸)(R^{8b}); X¹³ is C(R⁴⁰)(R⁴¹); n is 1; and v is 2. In some embodiments, R²³ is of Formula XLVI wherein X³ is C(R⁸); X⁴ is O; X¹⁰ is C(R³⁷)(R⁷); X¹¹ is C(R⁴); X¹² is C(R⁸)(R^{8b}); X¹³ is C(R⁴⁰)(R⁴¹); n is 1; and v is 1.

In some embodiments, R²³ is a 5 membered ring, i.e. X³, (X¹³)v, X¹¹, X⁴, X¹² and (X¹⁰)ₙ together form a 5 membered ring. For example, in some embodiments, R²³ is of Formula XLVI wherein n is 1, v is 1 and X⁴ is a bond; or n is 0, v is 1 and X⁴ is not a bond. In some embodiments, R²³ is of Formula XLVI wherein X³ is N; X¹³ is C(R⁴⁰)(R⁴¹); X¹¹ is C(R⁴); X⁴ is C(R³⁸)(R³⁹); X¹² is C(R⁶)(R^{6b}); and n is 0.

In some embodiments, R²³ is a 4 membered ring, i.e. X³, (X¹³)ᵥ, X¹¹, X⁴, X¹² and (X¹⁰)ₙ together form a 4 membered ring. For example, in some embodiments, R²³ is of Formula XLVI wherein n is 0, v is 1 and X⁴ is a bond; or n is 1, v is 0 and X⁴ is a bond. In some embodiments, R²³ is of Formula XLVI wherein X³ is N; X⁴ is a bond; n is 0; X¹¹ is C(R⁴); X¹² is C(R⁶)(R^{6b}); X¹³ is C(R⁴⁰)(R⁴¹); and v is 1.

In some embodiments, X³ is N. In some embodiments, X³ is C(R⁸), wherein R⁸ is selected from the group consisting of H, —OH, —O-alkyl and halogen. In some embodiments, R⁸ is H. In some embodiments, X³ is C(H).

In some embodiments, X⁴ is CH₂, O, CHF, CF₂, NH, NR⁹ or a bond, wherein R⁹ is as defined herein. In some embodiments, X⁴ is CH₂, O, CHF, NH or NR⁹, wherein R⁹ is as defined herein. In some embodiments, X⁴ is O. In some embodiments, X⁴ is CF₂. In some embodiments, X⁴ is CH₂. In some embodiments, X⁴ is CHF. In some embodiments, X⁴ is, NR⁹, wherein R⁹ is as defined herein. In some embodiments, R⁹ is C₁₋₆ alkyl, —C(O)—C₁₋₆ alkyl or —C(O)—C₃₋₆ cycloalkyl, or a pharmaceutically acceptable salt thereof.

In some embodiments, X⁴ is C(R³⁸)(R³⁹), wherein R³⁸ is H or F, and R³⁹ is H or F. In some embodiments, R³⁸ is H. In some embodiments, R³⁸ is F. In some embodiments R³⁹ is F. In some embodiments, R³⁹ is F. In some embodiments, R³⁸ is H and R³⁹ is H. In some embodiments, R³⁸ is F and R³⁹ is F. In some embodiments, R³⁸ is H and R³⁹ is F.

In some embodiments, R⁴ is C₁₋₃ alkyl. In some embodiments, R⁴ is H.

In some embodiments, R²³ is of Formula XVI, wherein X³ is N; X¹⁰ is CH₂; n is 1; v is 1; R⁶ is H; X⁴ is O; and X¹¹ is C(H). In some embodiments, R²³ is of Formula XX, wherein X³ is N; X¹⁰ is CH₂; n is 1; v is 1; X⁴ is O; and; X¹¹ is C(H). In some embodiments, the R²³ is of Formula XX, wherein X³ is N; X¹⁰ is CH₂; n is 1; v is 1; X⁴ is O; X¹¹ is C(H); X¹² is C(R⁶)(R^{6b}). In some embodiments, R²³ is of Formula XXXIV:

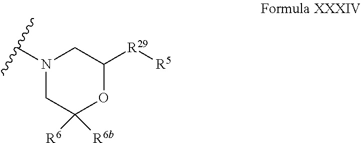

wherein R⁵, R⁶, R^{6b} and R²⁹ are as defined herein. In some embodiments, R²³ is of Formula XXXIV, R⁶ is H and R^{6b} is H. In some embodiments, R²³ is of Formula XXXIV, R⁶ is H and R^{6b} is C₁₋₃ alkyl. In some embodiments, R²³ is of Formula XXXIV, R⁶ is C₁₋₃ alkyl and R^{6b} is H. In some embodiments, R²³ is of Formula XXIV, R⁶ is C₁₋₃ alkyl and R^{6b} is C₁₋₃ alkyl. In some embodiments, R²³ is of Formula XXXIV, R⁶ is H and R^{6b} is halogen. In some embodiments, R²³ is of Formula XXXIV, R⁶ is halogen and R^{6b} is halogen. In some embodiments, R²³ is of Formula XXXIV, R⁶ is H and R^{6b} is H. In some embodiments, R²³ is of Formula XXXIV, R⁶ is F and R^{6b} is F. In some embodiments, R²³ is of Formula XXXIV, R⁶ is CH₃ and R^{6b} is CH₃. In some embodiment, R²³ is of Formula XXXIV, R⁶ is CH₃ and R^{6b} is H.

In some embodiments, R²³ is of Formula XXXIV, R⁶ is C₁₋₃ alkyl and R^{6b} is C₁₋₃ alkyl, and R⁶ and R^{6b} are linked together to form a 3 to 6-membered ring. In some embodiments, R²³ is of Formula XXXIV, R⁶ is C₁₋₃ alkyl and R^{6b} is C₁₋₃ alkyl, and R⁶ and R^{6b} are linked together to form a 4-membered ring, for example R⁶ is C₁ alkyl and R^{6b} is C₂ alkyl or R⁶ is C₂ alkyl and R^{6b} is C₁ alkyl. In some embodiments, R²³ is of Formula XXXIV, R⁶ is C₁₋₃ alkyl and R^{6b} is C₁₋₃ alkyl, and R⁶ and R^{6b} are linked together to form a 3-membered ring, for example R⁶ is C₁ alkyl and R^{6b} is C₁ alkyl. In some embodiments, R⁶ and R^{6b} are C₁₋₃ alkyl, R⁶ and R^{6b} are linked together to form a 3-6 membered ring, and one methylene group is optionally replaced with —O—. In some embodiments, R⁶ and R^{6b} are C₁₋₃ alkyl, R⁶ and R^{6b} are linked together to form a 3-6 membered ring, and one methylene group is optionally replaced with —N(R³⁵)—, wherein R³⁵ is selected from the group consisting of H, C₁₋₃ alkyl and —C(O)C₁₋₃ alkyl. In some embodiments, R³⁵ is H. In some embodiments, R³⁵ is C₁₋₃ alkyl. In some embodiments, R³⁵ is —C(O)C₁₋₃ alkyl. In some embodiments, R²³ is of Formula XLV:

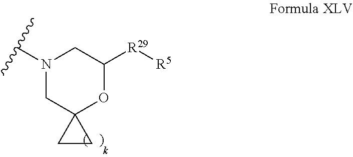

wherein k is 1, 2, 3 or 4. In some embodiments, R²³ is of Formula XLV wherein k is 1. In some embodiments, R²³ is of Formula XLV wherein k is 2.

In some embodiments, R²³ is of Formula XLVI, wherein X³ is C(R⁸); R⁸ C₁ alkyl; X¹³ is C(R⁴⁰)(R⁴¹); R⁴⁰ is a bond; R⁸ and R⁴⁰ are linked together to form a 3-membered ring; R⁴¹ is H; and v is 1. In some embodiments, R²³ is of Formula LV:

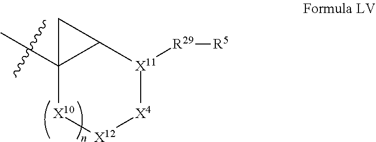

wherein R⁵, R²⁹, X⁴, X¹⁰, X¹² and n are as defined herein.

In some embodiments, R²³ is of Formula LVI, wherein R⁵, R²⁹, X⁴, X¹⁰, X¹² and n are as defined herein. In some embodiments, R²³ is of Formula LVI, wherein R⁵, R²⁹, X⁴, X¹⁰, X¹² and n are as defined herein. In some embodiments, R²³ is of Formula LVIII, wherein R⁵, R²⁹, X⁴, X¹⁰, X¹² and n are as defined herein. In some embodiments, R²³ is of Formula LIX, wherein R⁵, R²⁹, X⁴, X¹⁰, X¹² and n are as defined herein.

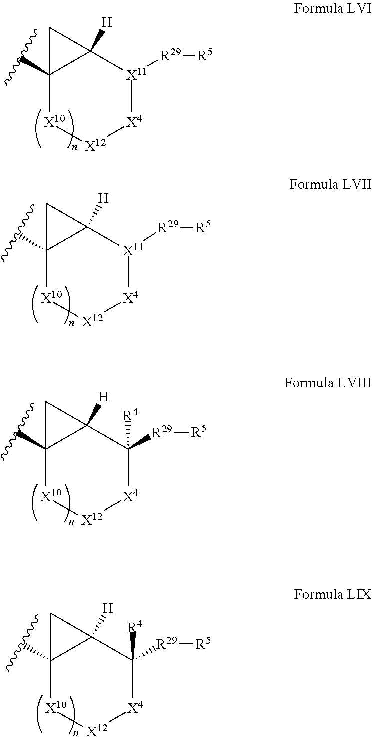

In some embodiments, R²³ is of Formula XLVI, wherein X³ is C(R⁸); R³ is a bond; X¹³ is C(R⁴⁰)(R⁴¹); R⁴⁰ is C₁ alkyl; R³ and R⁴⁰ are linked together to form a 3-membered ring; R⁴¹ is H; v is 1; X¹¹ is C(H), X⁴ is O; X¹² is CH₂; X¹⁰ is CH₂; and n is 1. In some embodiments, R²³ is of Formula LX:

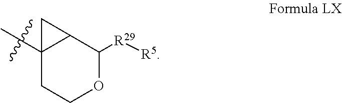

wherein R⁵ and R²⁹ are as defined herein.

In some embodiments, R²³ is of Formula XLVI, wherein X¹² is C(R⁶)(R^{6b}); R^{6b} is H; R⁶ is C₁₋₃ alkyl; n is 1; X¹⁰ is C(R³⁷)(R⁷); R³⁷ is H; and R⁷ is a bond to R⁶ thus forming a 3-5 membered ring. In some embodiments, R²³ is of Formula XLVI, wherein X³ is N; X¹³ is CH₂; v is 1; X¹¹ is C(H); X⁴ is O; X¹² is C(R⁶)(R^{6b}); R^{6b} is H; R⁶ is C₁ alkyl; n is 1; X¹⁰ is C(R³⁷)(R⁷); R³⁷ is H; and R⁷ is a bond to R⁶ forming a 3 membered ring. In some embodiments, R²³ is of Formula LXIII:

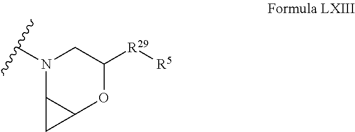

wherein R⁵ and R²⁹ are as defined herein.

In some embodiments, R²³ is of Formula XVI, wherein X³ is N; X¹⁰ is CH₂; n is 1; v is 1; R⁶ is H; X⁴ is C(R³³)(R³⁴); R³³ is H or F; R³⁴ is H or F; and; X¹¹ is C(H). In some embodiments, R²³ is of Formula XXXV:

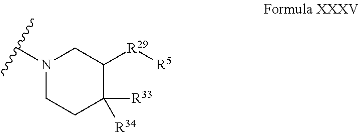

wherein R⁵ and R²⁹ are as defined herein, R³³ is H or F, and R³⁴ is H or F. In some embodiments, R²³ is of Formula XXXV, R³³ is H and R³⁴ is H. In some embodiments, R²³ is of Formula XXXV, R³³ is F and R³⁴ is F.

In some embodiments, R²³ is of Formula XVI, wherein X³ is N; n is 0; v is 1; R⁶ is H; X⁴ is CH₂; and; X¹¹ is C(H). In some embodiments, R²³ is of Formula XXXVI:

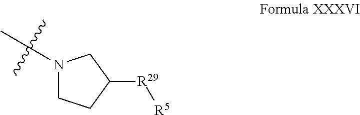

wherein R⁵ and R²⁹ are as defined herein.

In some embodiments, R²³ is of Formula XVI, wherein X³ is N; X¹⁰ is CH₂; n is 1; v is 1; R⁶ is H; X⁴ is N(R⁹); and; X¹¹ is C(H). In some embodiments, R²³ is of Formula XXXVII:

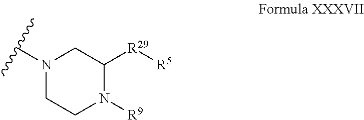

wherein R⁵, R⁹ and R²⁹ are as defined herein.

In some embodiments, R²³ is of Formula XVI, wherein X³ is C(H); X¹⁰ is CH₂; n is 1; v is 1; R⁶ is H; X⁴ is O; and; X¹¹ is C(H). In some embodiments, R²³ is of Formula XXXVIII:

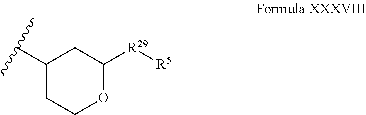

wherein R⁵ and R²⁹ are as defined herein.

In some embodiments, R²³ is of Formula XVI, wherein X³ is C(H); X¹⁰ is CH₂; n is 1; v is 1; R⁶ is H; X⁴ is C(R³³)(R³⁴); R³³ is H or F; R³⁴ is H or F; and X¹¹ is N. In some embodiments, R²³ is of Formula XXXIX:

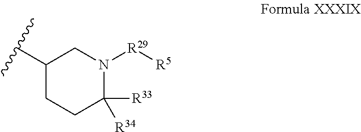

wherein R⁵ and R²⁹ are as defined herein, R³³ is H or F, and R³⁴ is H or F. In some embodiments, R²³ is of Formula XXXIX, R³³ is H and R³⁴ is H. In some embodiments, R²³ is of Formula XXXIX, R³³ is F and R³⁴ is F.

In some embodiments, R²³ is of Formula XVI, wherein X³ is N; n is 0; v is 1; R⁶ is H; X⁴ is a bond; and X¹¹ is C(H). In some embodiments, R²³ is of Formula XL:

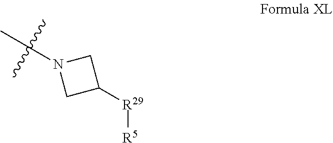

wherein R⁵ and R²⁹ are as defined herein.

In some embodiments R²³ is of Formula XVI, wherein X³ is N; X¹⁰ is CH₂; n is 1; v is 2; R⁶ is H; X⁴ is a bond; and X¹¹ is C(H). In some embodiments, R²³ is of Formula XLI:

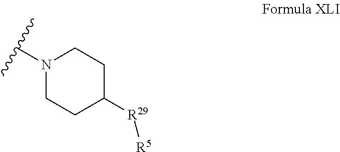

wherein R⁵ and R²⁹ are as defined herein.

In some embodiments, R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
   wherein the C₁₋₆ alkyl or C₁₋₆ haloalkyl of subsection (b) are optionally substituted with —OH, and
   wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O) and —N(R³⁰)(R³¹),
   wherein R²⁴ is C₁₋₆ alkyl or aryl, wherein the C₁₋₆ alkyl is optionally substituted with C₁₋₃ alkoxy or halogen;
   R³⁰ is H or C₁₋₃ alkyl; and
   R³¹ is H or C₁₋₃ alkyl.

In some embodiments, R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with oxo (═O), and
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl); and
(c) the C₁₋₆ alkyl is optionally substituted with —N(H)C(O)R²⁴, wherein R²⁴ is C₁₋₆ alkyl or aryl, wherein the C₁₋₆ alkyl is optionally substituted with C₁₋₃ alkoxy or halogen.

In some embodiments, R⁵ is C₃₋₆ cycloalkyl or C₃₋₆ heterocycloalkyl, wherein the C₃₋₆ cycloalkyl or C₃₋₆ heterocycloalkyl is optionally substituted with 1 to 3 substituents independently selected from oxo (═O), C₁₋₃ alkoxy and halogen.

In some embodiments, R⁵ is C₃₋₆ cycloalkyl or C₃₋₆ heterocycloalkyl, and the C₃₋₆ cycloalkyl or C₃₋₆ heterocycloalkyl is optionally substituted with oxo (═O), or a pharmaceutically acceptable salt thereof. In some embodiments, R⁵ is C₁₋₆ alkyl, wherein the C₁₋₆ alkyl is optionally substituted with —N(H)C(O)R²⁴, wherein R²⁴ is C₁₋₆ alkyl or aryl, wherein the C₁₋₆ alkyl is optionally substituted with C₁₋₃ alkoxy or halogen, or a pharmaceutically acceptable salt thereof. In some embodiments, R⁵ is C₁₋₆ alkyl, wherein the C₁₋₆ alkyl is optionally substituted with -oxo (═O). In some embodiments, R⁵ is C₁₋₆ alkyl, wherein the C₁₋₆ alkyl is optionally substituted with —N(R³⁰)(R³¹), wherein R³⁰ is H or C₁₋₃ alkyl, and R³¹ is H or C₁₋₃ alkyl. In some embodiments, R³⁰ is H.

In some embodiments, R³⁰ is C₁₋₃ alkyl, such as C₁ alkyl. In some embodiments, R³¹ is H. In some embodiments, R³¹ is C₁₋₃ alkyl, such as C₁ alkyl. In some embodiments, R³⁰ is C₁₋₃ alkyl, such as C₁ alkyl, and R³¹ is H. In some embodiments, R³⁰ is C₁₋₃ alkyl, such as C₁ alkyl, and R³¹ is C₁₋₃ alkyl, such as C₁ alkyl.

In some embodiments, R⁵ is C₃₋₆ heterocycloalkyl optionally substituted with 1 to 3 substituents independently selected from oxo (═O), C₁₋₃ alkoxy and halogen. In some embodiments, R⁵ is C₄ heterocycloalkyl optionally substituted with 1 to 3 substituents independently selected from C₁₋₃ alkoxy and halogen. In some embodiments, R⁵ is an azetidine optionally substituted with 1 to 3 substituents independently selected from C₁₋₃ alkoxy and halogen. In some embodiments, R⁵ is

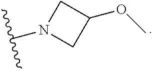 [SMILES: *N1CC(OC)C1]

In some embodiments, R⁵ is

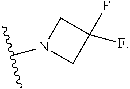 [SMILES: *N1CC(F)(F)C1]

In some embodiments, R⁵ is selected from the group consisting of phenyl, —O-phenyl, 5-membered heteroaryl, —O-(5-membered heteroaryl), 6-membered heteroaryl and —O-(6-membered heteroaryl), wherein the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃-6 heterocycloalkyl,
wherein the C₁₋₆ alkyl or C₁₋₆ haloalkyl are optionally substituted with —OH, and
wherein the C₃₋₆ heterocycloalkyl is optionally substituted with 1 to 3 substituents individually selected from the group consisting of halogen, C₁₋₃ alkyl and —C(O)O(C₁₋₆ alkyl).

In some embodiments, R⁵ is a 5-membered heteroaryl optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with 1 to 3 substituents independently selected from the group consisting of C₁₋₆ alkyl, C₁₋₆ alkoxy, halogen and —OH,
and wherein the C₃₋₆ heterocycloalkyl is optionally substituted with 1 to 3 substituents individually selected from the group consisting of halogen, C₁₋₃ alkyl and —C(O)O(C₁₋₆ alkyl).

In some embodiments, R⁵ is 5-membered heteroaryl optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and wherein the C₃₋₆ heterocycloalkyl is optionally substituted with 1 to 3 substituents individually selected from the group consisting of halogen, C₁₋₃ alkyl and —C(O)O(C₁₋₆ alkyl).

In some embodiments, R⁵ is of Formula XXI:

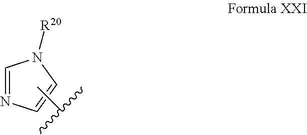

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy.

In some embodiments, R⁵ is of Formula XXII:

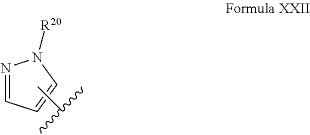

wherein
R²⁰ is C₁₋₃ alkyl, C₃₋₆ cycloalkyl, H or C₃₋₆ heterocycloalkyl, wherein the C₁₋₃ alkyl, C₃₋₆ cycloalkyl or C₃ heterocycloalkyl is optionally substituted with 1 to 3 substituents independently selected from the group consisting of C₁₋₆ alkyl C₁₋₆ alkoxy, halogen and —OH.

In some embodiments, R⁵ is of Formula XXII:

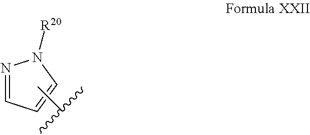

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula XXIII:

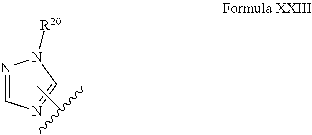

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy.

In some embodiments, R⁵ is of Formula XXIV:

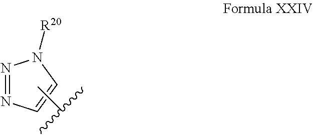

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula XLIII:

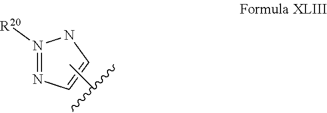

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is

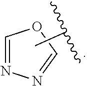

In some embodiments, R⁵ is

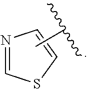

In some embodiments, R⁵ is of Formula VII:

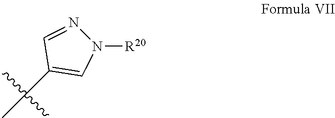

wherein
R²⁰ is C₁₋₃ alkyl, C₃₋₆ cycloalkyl, H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl C₁₋₆ alkoxy, halogen and —OH.

In some embodiments, R⁵ is of Formula VII:

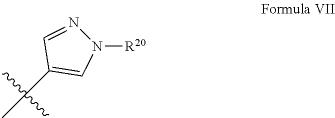

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula VII:

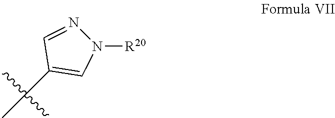

wherein
R²⁰ is C₁₋₃ alkyl, such as —CH₃, or C₃₋₆ cycloalkyl, such as C₃ cycloalkyl.

In some embodiments, R⁵ is of Formula XXV:

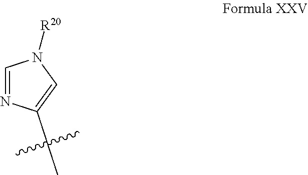

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula XXVI:

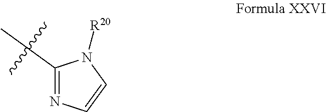

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula XXVII:

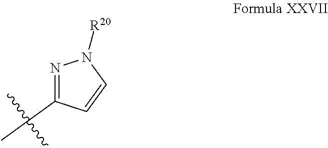

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula XXVIII:

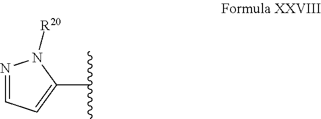

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula XXIX:

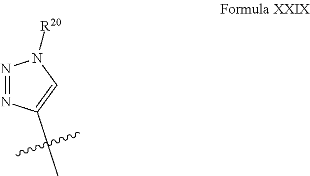

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy.

In some embodiments, R⁵ is of Formula XXX:

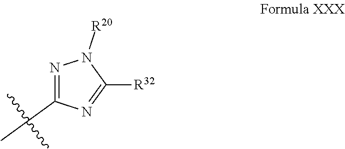

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy; and R³² is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In one embodiment, R⁵ is of Formula XLII:

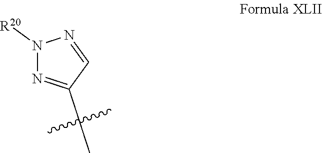

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy.

In some embodiments, R⁵ is of Formula XXXI:

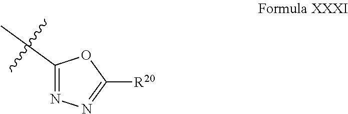

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy.

In some embodiments, R⁵ is of Formula XXXII:

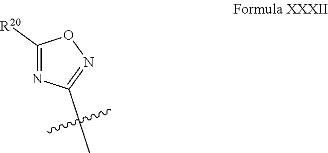

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R⁵ is of Formula XXXIII:

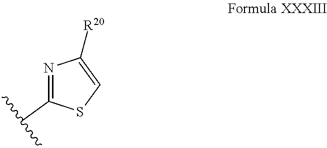

wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy.

In some embodiments, R²⁰ is C₁₋₃ alkyl, such as —CH₃. In some embodiments, R²⁰ is C₁₋₃ alkyl substituted with C₁₋₆ alkoxy. In some embodiments, R²⁰ is C₁₋₂ alkyl optionally substituted with C, alkoxy. In some embodiments, R²⁰ is —CH₂CH₂OCH₃. In some embodiments, R²⁰ C₃₋₆ cycloalkyl, such as C₃ cycloalkyl. In some embodiments, R²⁰ is H. In some embodiments, R²⁰ is C₁₋₃ alkyl optionally substituted with 1 to 3 halogens, such as F. In some embodiments, R²⁰ is methyl optionally substituted with 1 to 3 halogens, such as F. In some embodiments, R²⁰ is —CF₃.

In some embodiments, R⁵ is

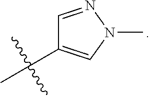 [SMILES: *c1cnn(C)c1]

In some embodiments, R⁵ is

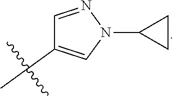 [SMILES: *c1cnn(C2CC2)c1]

In some embodiments, R⁵ is

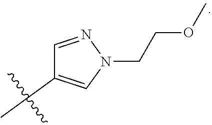 [SMILES: *c1cnn(CCOC)c1]

In some embodiments, R⁵ is

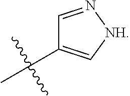 [SMILES: *c1cn[nH]c1]

In some embodiments, R⁵ is

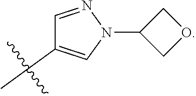 [SMILES: *c1cnn(C2COC2)c1]

In some embodiments, R⁵ is

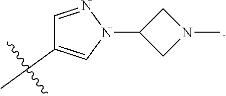 [SMILES: *c1cnn(C2CN(C)C2)c1]

In some embodiments, R is

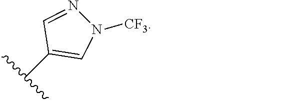 [SMILES: *c1cnn(C(F)(F)F)c1]

In some embodiments, R⁵ is

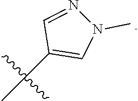 [SMILES: *c1cnn(C)c1]

In some embodiments, R⁵ is

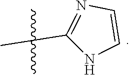 [SMILES: *c1ncc[nH]1]

In some embodiments, R⁵ is

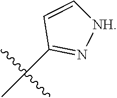 [SMILES: *c1cc[nH]n1]

In some embodiments, R⁵ is

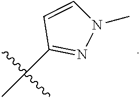 [SMILES: *c1ccn(C)n1]

In some embodiments, R⁵ is

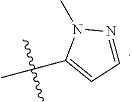 [SMILES: *c1ccnn1C]

In some embodiments, R⁵ is

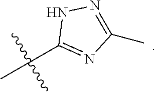 [SMILES: *c1nc(C)n[nH]1]

In some embodiments, R⁵ is

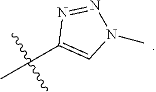 [SMILES: *c1cn(C)nn1]

In some embodiments, R⁵ is

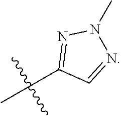 [SMILES: *c1cnn(C)n1]

In some embodiments, R⁵ is

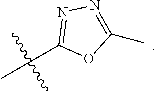 [SMILES: *c1nnc(C)o1]

In some embodiments, R⁵ is

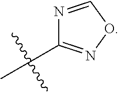 [SMILES: *c1ncon1]

In some embodiments, R⁵ is

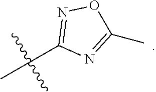 [SMILES: *c1noc(C)n1]

In some embodiments, R⁵ is

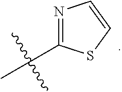 [SMILES: *c1nccs1]

In some embodiments, R⁵ is

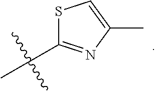 [SMILES: *c1nc(C)cs1]

In some embodiments, R⁵ is 6-membered heteroaryl optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and wherein the C₃₋₆ heterocycloalkyl is optionally substituted with 1 to 3 substituents individually selected from the group consisting of halogen, C₁₋₃ alkyl and —C(O)O(C₁₋₆ alkyl).

In some embodiments, X¹² is CH₂, R²⁹ is a bond and R⁵ is pyrazolyl optionally substituted with C₁₋₆ alkyl or C₃₋₆ cycloalkyl. In some embodiments, X¹² is CH₂, R²⁹ is a bond and R⁵ is pyrazolyl substituted with C₃₋₆ cycloalkyl, such as cyclopropyl.

In some embodiments, R⁵ is of Formula XLIV:

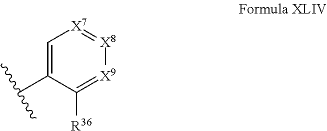

wherein
X⁷ is N or CH;
X¹ is N or C(R²¹);
X⁹ is N or C(R²²);
R²¹ is C₁₋₃ alkyl, such as —CH₃;
R²² is C₁₋₃ alkyl, such as —CH₃, and
R³¹ is H or C₁₋₃ alkyl, such as —CH₃.

In some embodiments, R⁵ is of Formula VIII:

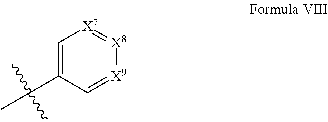

wherein
X⁷ is N or CH;
X¹ is N or C(R²¹);
X⁹ is N or C(R²²);
R²¹ is C₁₋₃ alkyl, such as —CH₃; and
R²² is C₁₋₃ alkyl, such as —CH₃,

In some embodiments, R⁵ is selected from the group consisting of

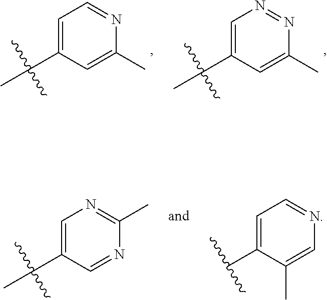

In some embodiments, R⁵ is C₁₋₆ alkyl optionally substituted with 1 to 3 substituents independently selected from —N(H)C(O)R²⁴, -oxo (═O) and —N(R³⁰)(R³¹),
wherein R²⁴ is C₁₋₆ alkyl or aryl, wherein the C₁-6 alkyl is optionally substituted with C₁₋₃ alkoxy or halogen;
R³⁰ is H or C₁₋₃ alkyl; and
R³¹ is H or C₁₋₃ alkyl.

In some embodiments, R⁵ is of Formula XIII:

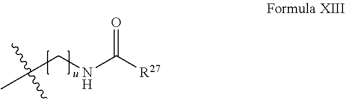

wherein
R²⁷ is C₁₋₆ alkyl; and
u is 1, 2 or 3.

In some embodiments, R⁴ and R⁵ are linked together to form a ring, thus generating a spiro compound. In some embodiments, R⁴ is C₁₋₃ alkyl and R⁵ is C₁₋₃ alkyl. In some embodiments, R⁴ is C₁₋₃ alkyl and R⁵ is C₁₋₃ alkyl and R⁴ and R⁵ are linked together to form a 3 to 6-membered ring. In some embodiments, R⁴ is C₂ alkyl and R⁵ is C₂ alkyl and R⁴ and R⁵ are linked together to form a 4-membered ring.

In some embodiments, R⁶ is C₁₋₃ alkyl. In some embodiments, R⁶ is H. In some embodiments, R⁶ is halogen. In some embodiments, R^{6b} is C₁₋₃ alkyl. In some embodiments, R^{6b} is H. In some embodiments, R^{6b} is halogen. In some embodiments, R⁶ and R^{6b} are H. In some embodiments, R⁶ is halogen and R^{6b} is halogen. In some embodiments, R⁶ is H and R^{6b} is halogen. In some embodiments, R⁶ and R^{6b} are C₁₋₃ alkyl, such as C₁ alkyl. In some embodiments, R⁶ and R^{6b} are linked together to form a ring, thus generating a spiro compound. In some embodiments, R⁶ and R^{6b} are C₁₋₃ alkyl, and R⁶ and R^{6b} are linked together to form a 3-6 membered ring, such as a 4-membered ring or a 3-membered ring. In some embodiments, R⁶ and R^{6b} are C₁ alkyl and R⁶ and R^{6b} are linked together to form a 3-membered ring. In some embodiments, R⁶ is C₁ alkyl and R^{6b} C₂ alkyl or R⁶ is C₂ alkyl and R^{6b} C₁ alkyl, and R⁶ and R^{6b} are linked together to form a 4-membered ring. In some embodiments, R⁶ and R^{6b} are C₁₋₃ alkyl, R⁶ and R^{6b} are linked together to form a 3-6 membered ring, and one methylene group is optionally replaced with —O—. In some embodiments, R⁶ and R^{6b} are C₁₋₃ alkyl, R⁶ and R^{6b} are linked together to form a 3-6 membered ring, and one methylene group is optionally replaced with —N(R³⁵)—, wherein R³⁵ is selected from the group consisting of H, C₁₋₃ alkyl and —C(O)C₁₋₃ alkyl. In some embodiments, R³⁵ is H. In some embodiments, R³⁵ is C₁₋₃ alkyl. In some embodiments, R³⁵ is —C(O)C₁₋₃ alkyl.

In some embodiments, X¹⁰ is C(H)(R⁷) or C(O). In some embodiments, X¹⁰ is C(H)(R⁷), wherein R⁷ is H or C₁₋₃ alkyl. In some embodiments, X¹⁰ is individually C(R³⁷)(R⁷), wherein R⁷ is individually H or C₁₋₃ alkyl, and R³⁷ is individually H or C₁₋₃ alkyl. In some embodiments, R⁷ is H. In some embodiments, R³⁷ is H. In some embodiments, R⁷ is H and R³⁷ is H. In some embodiments, X¹⁰ is C(H)₂. When R⁷ is a bond, it forms a bond between the C of X¹⁰ and another atom, such as a C of R⁶. In some embodiments, R⁷ is a bond and then R⁶ is C₁₋₃ alkyl, and R⁷ and R⁶ are linked together to form a 3-5 membered ring.

In some embodiments, X¹³ is C(R⁴⁰)(R⁴¹), wherein R⁴⁰ is individually H, C₁₋₃ alkyl or a bond, and R⁴¹ is individually H or C₁₋₃ alkyl. In some embodiments, R⁴⁰ is H. In some embodiments, R⁴¹ is H. In some embodiments, R⁴⁰ is H and R⁴¹ is H. In some embodiments, X¹³ is CH₂. When R⁴⁰ is a bond, it forms a bond between the C of X¹³ and another atom, such as a C of R⁸. In some embodiments, when R⁴⁰ is a bond, then R⁸ is C₁₋₃ alkyl, and R⁸ and R⁴⁰ are linked together to form a 3-5 membered ring.

In some embodiments, R²³ is

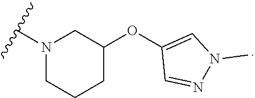 [SMILES: *N1CCCC(Oc2cnn(C)c2)C1]

In some embodiments, R²³ is

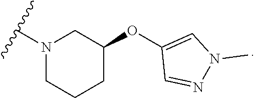 [SMILES: *N1CCC[C@H](Oc2cnn(C)c2)C1]

In some embodiments, R²³ is

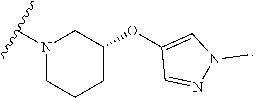 [SMILES: *N1CCC[C@@H](Oc2cnn(C)c2)C1]

In some embodiments, R²³ is

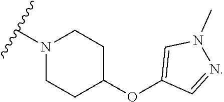 [SMILES: *N1CCC(Oc2cnn(C)c2)CC1]

In some embodiments, R²³ is

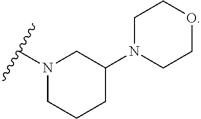 [SMILES: *N1CCCC(N2CCOCC2)C1]

In some embodiments, R²³ is

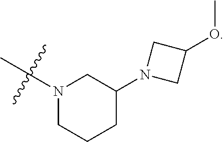 [SMILES: *N1CCCC(N2CC(OC)C2)C1]

In some embodiments, R²³ is

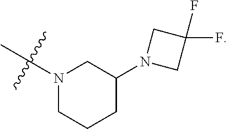 [SMILES: *N1CCCC(N2CC(F)(F)C2)C1]

In some embodiments, R²³ is

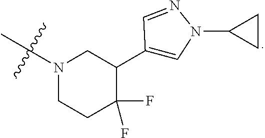 [SMILES: *N1CCC(F)(F)C(c2cnn(C3CC3)c2)C1]

In some embodiments, R²³ is

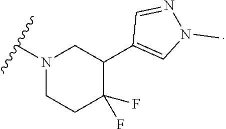 [SMILES: *N1CCC(F)(F)C(c2cnn(C)c2)C1]

In some embodiments, R²³ is

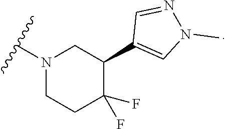 [SMILES: *N1CCC(F)(F)[C@H](c2cnn(C)c2)C1]

In some embodiments, R²³ is

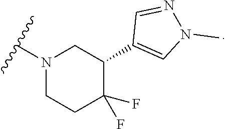 [SMILES: *N1CCC(F)(F)[C@@H](c2cnn(C)c2)C1]

In some embodiments, R²³ is

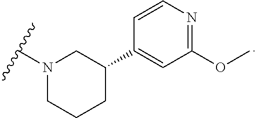 [SMILES: *N1CCC[C@@H](c2ccnc(OC)c2)C1]

In some embodiments, R²³ is

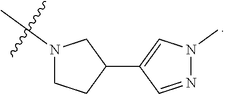 [SMILES: *N1CCC(c2cnn(C)c2)C1]

In some embodiments, R²³ is

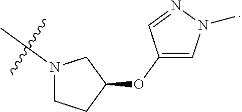 [SMILES: *N1CC[C@H](Oc2cnn(C)c2)C1]

In some embodiments, R²³ is

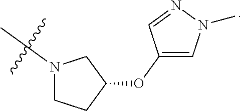 [SMILES: *N1CC[C@@H](Oc2cnn(C)c2)C1]

In some embodiments, R²³ is

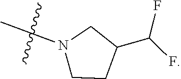 [SMILES: *N1CCC(C(F)F)C1]

In some embodiments, R²³ is

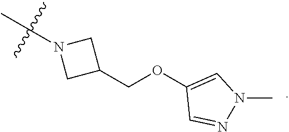 [SMILES: *N1CC(COc2cnn(C)c2)C1]

In some embodiments, R²³ is

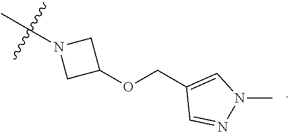 [SMILES: *N1CC(OCc2cnn(C)c2)C1]

In some embodiments, R²³ is

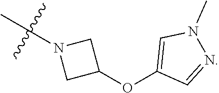 [SMILES: *N1CC(Oc2cnn(C)c2)C1]

In some embodiments, R²³ is

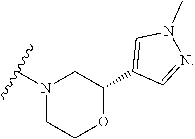 [SMILES: *N1CCO[C@@H](c2cnn(C)c2)C1]

In some embodiments, R²³ is

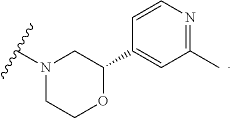 [SMILES: *N1CCO[C@@H](c2ccnc(C)c2)C1]

In some embodiments, R²³ is

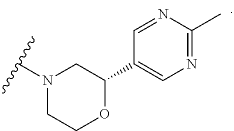 [SMILES: *N1CCO[C@@H](c2cnc(C)nc2)C1]

In some embodiments, R²³ is

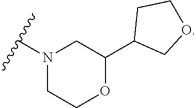 [SMILES: *N1CCOC(C2CCOC2)C1]

In some embodiments, R²³ is

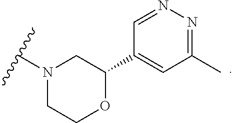 [SMILES: *N1CCO[C@@H](c2cnnc(C)c2)C1]

In some embodiments, R²³ is

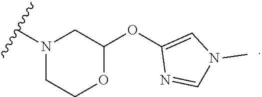 [SMILES: *N1CCOC(Oc2cn(C)cn2)C1]

In some embodiments, R²³ is

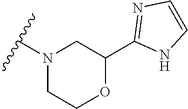 [SMILES: *N1CCOC(c2ncc[nH]2)C1]

In some embodiments, R²³ is

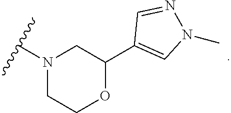 [SMILES: *N1CCOC(c2cnn(C)c2)C1]

In some embodiments, R²³ is

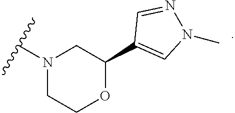 [SMILES: *N1CCO[C@H](c2cnn(C)c2)C1]

In some embodiments, R²³ is

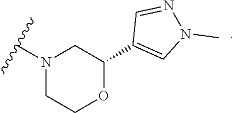 [SMILES: *N1CCO[C@@H](c2cnn(C)c2)C1]

In some embodiments, R²³ is

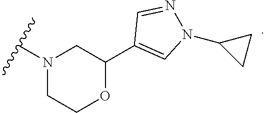 [SMILES: *N1CCOC(c2cnn(C3CC3)c2)C1]

In some embodiments, R²³ is

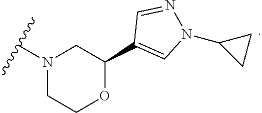 [SMILES: *N1CCO[C@H](c2cnn(C3CC3)c2)C1]

In some embodiments, R²³ is

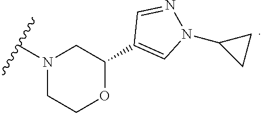 [SMILES: *N1CCO[C@@H](c2cnn(C3CC3)c2)C1]

In some embodiments, R²³ is

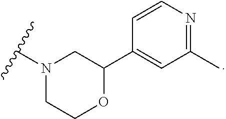 [SMILES: *N1CCOC(c2ccnc(C)c2)C1]

In some embodiments, R²³ is

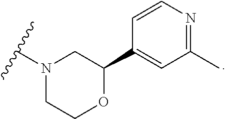 [SMILES: *N1CCO[C@H](c2ccnc(C)c2)C1]

In some embodiments, R²³ is

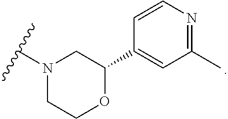 [SMILES: *N1CCO[C@@H](c2ccnc(C)c2)C1]

In some embodiments, R²³ is

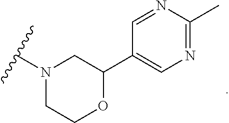 [SMILES: *N1CCOC(c2cnc(C)nc2)C1]

In some embodiments, R²³ is

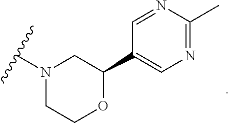 [SMILES: *N1CCO[C@H](c2cnc(C)nc2)C1]

In some embodiments, R²³ is

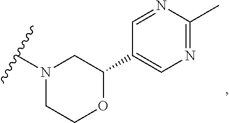 [SMILES: *N1CCO[C@@H](c2cnc(C)nc2)C1]

In some embodiments, R²³ is

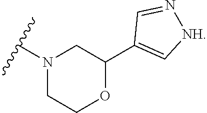 [SMILES: *N1CCOC(c2cn[nH]c2)C1]

In some embodiments, R²³ is

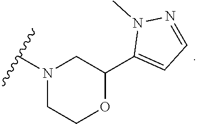 [SMILES: *N1CCOC(c2ccnn2C)C1]

In some embodiments, R²³ is

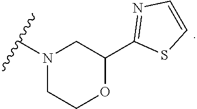 [SMILES: *N1CCOC(c2nccs2)C1]

In some embodiments, R²³ is

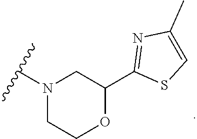 [SMILES: *N1CCOC(c2nc(C)cs2)C1]

In some embodiments, R²³ is

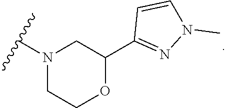 [SMILES: *N1CCOC(c2ccn(C)n2)C1]

In some embodiments, R²³ is

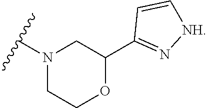 [SMILES: *N1CCOC(c2cc[nH]n2)C1]

In some embodiments, R²³ is

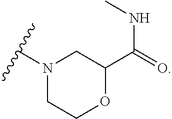 [SMILES: *N1CCOC(C(=O)NC)C1]

In some embodiments, R²³ is

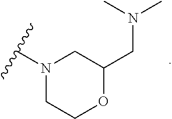 [SMILES: *N1CCOC(CN(C)C)C1]

In some embodiments, R²³ is

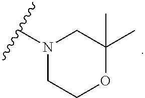 [SMILES: *N1CCOC(C)(C)C1]

In some embodiments, R²³ is

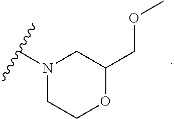 [SMILES: *N1CCOC(COC)C1]

In some embodiments, R²³ is

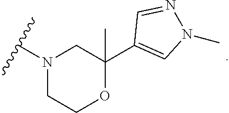 [SMILES: *N1CCOC(C)(c2cnn(C)c2)C1]

In some embodiments, R²³ is

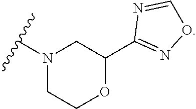 [SMILES: *N1CCOC(c2ncon2)C1]

In some embodiments, R²³ is

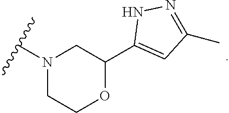 [SMILES: *N1CCOC(c2cc(C)n[nH]2)C1]

In some embodiments, R²³ is

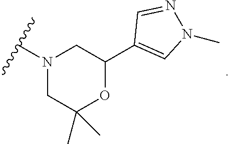 [SMILES: *N1CC(c2cnn(C)c2)OC(C)(C)C1]

In some embodiments, R²³ is

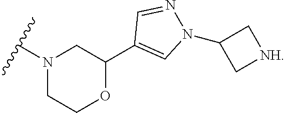 [SMILES: *N1CCOC(c2cnn(C3CNC3)c2)C1]

In some embodiments, R²³ is

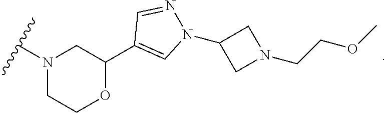 [SMILES: *N1CCOC(c2cnn(C3CN(CCOC)C3)c2)C1]

In some embodiments, R²³ is

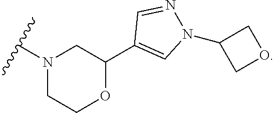 [SMILES: *N1CCOC(c2cnn(C3COC3)c2)C1]

In some embodiments, R²³ is

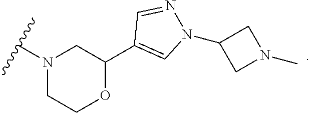 [SMILES: *N1CCOC(c2cnn(C3CN(C)C3)c2)C1]

In some embodiments, R² is

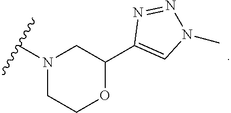 [SMILES: *N1CCOC(c2cn(C)nn2)C1]

In some embodiments, R²³ is

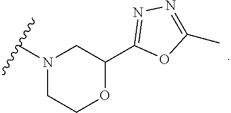 [SMILES: *N1CCOC(c2nnc(C)o2)C1]

In some embodiments, R²³ is

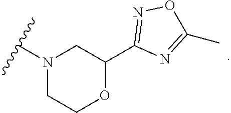 [SMILES: *N1CCOC(c2noc(C)n2)C1]

In some embodiments, R²³ is

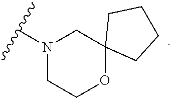 [SMILES: *N1CCOC2(CCCC2)C1]

In some embodiments, R²³ is

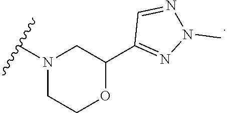 [SMILES: *N1CCOC(c2cnn(C)n2)C1]

In some embodiments, R²³ is

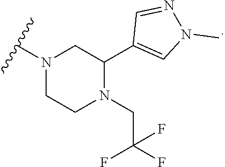 [SMILES: *N1CCN(CC(F)(F)F)C(c2cnn(C)c2)C1]

In some embodiments, R²³ is

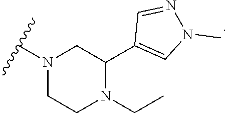 [SMILES: *N1CCN(CC)C(c2cnn(C)c2)C1]

In some embodiments, R²³ is

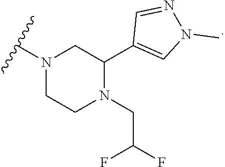 [SMILES: *N1CCN(CC(F)F)C(c2cnn(C)c2)C1]

In some embodiments, R²³ is

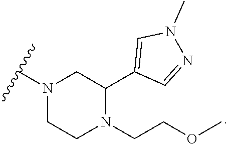 [SMILES: *N1CCN(CCOC)C(c2cnn(C)c2)C1]

In some embodiments, R²³ is

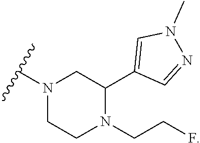 [SMILES: *N1CCN(CCF)C(c2cnn(C)c2)C1]

In some embodiments, R²³ is

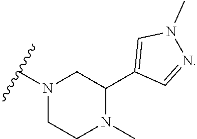 [SMILES: *N1CCN(C)C(c2cnn(C)c2)C1]

In some embodiments, R²³ is

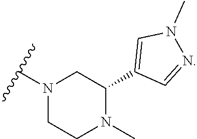 [SMILES: *N1CCN(C)[C@@H](c2cnn(C)c2)C1]

In some embodiments, R²³ is

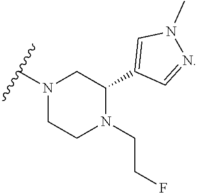 [SMILES: *N1CCN(CCF)[C@@H](c2cnn(C)c2)C1]

In some embodiments, R²³ is

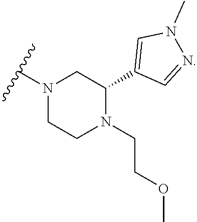 [SMILES: *N1CCN(CCOC)[C@@H](c2cnn(C)c2)C1]

In some embodiments, R²³ is

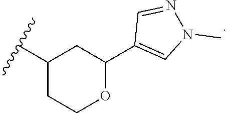 [SMILES: *C1CCOC(c2cnn(C)c2)C1]

In some embodiments, R²³ is

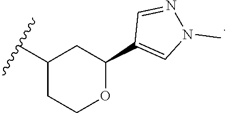 [SMILES: *C1CCO[C@H](c2cnn(C)c2)C1]

In some embodiments, R²³ is

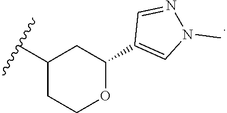 [SMILES: *C1CCO[C@@H](c2cnn(C)c2)C1]

In some embodiments, R²³ is

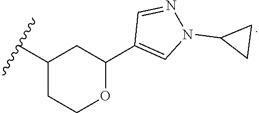 [SMILES: *C1CCOC(c2cnn(C3CC3)c2)C1]

In some embodiments, R²³ is

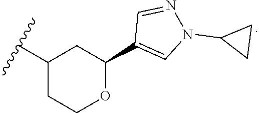 [SMILES: *C1CCO[C@H](c2cnn(C3CC3)c2)C1]

In some embodiments, R²³ is

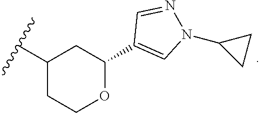 [SMILES: *C1CCO[C@@H](c2cnn(C3CC3)c2)C1]

In some embodiments, R²³ is

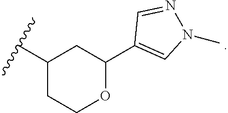 [SMILES: *C1CCOC(c2cnn(C)c2)C1]

In some embodiments, R²³ is

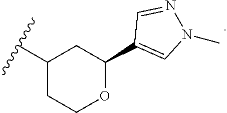 [SMILES: *C1CCO[C@H](c2cnn(C)c2)C1]

In some embodiments, R²³ is

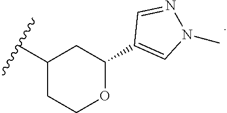 [SMILES: *C1CCO[C@@H](c2cnn(C)c2)C1]

In some embodiments, R²³ is

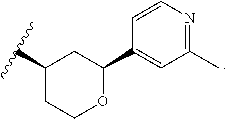 [SMILES: *[C@@H]1CCO[C@H](c2ccnc(C)c2)C1]

In some embodiments, R²³ is

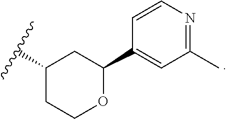 [SMILES: *[C@H]1CCO[C@H](c2ccnc(C)c2)C1]

In some embodiments, R²³ is

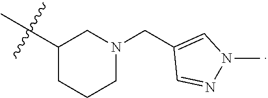 [SMILES: *C1CCCN(Cc2cnn(C)c2)C1]

In some embodiments, R²³ is

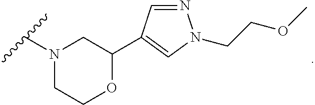 [SMILES: *N1CCOC(c2cnn(CCOC)c2)C1]

In some embodiments, R²³ is

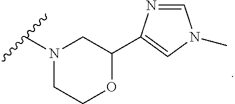 [SMILES: *N1CCOC(c2cn(C)cn2)C1]

In some embodiments, R²³ is

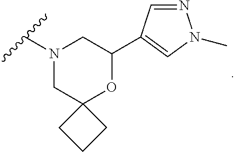 [SMILES: *N1CC(c2cnn(C)c2)OC2(CCC2)C1]

In some embodiments, R²³ is

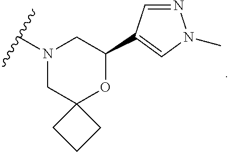 [SMILES: *N1C[C@@H](c2cnn(C)c2)OC2(CCC2)C1]

In some embodiments, R²³ is

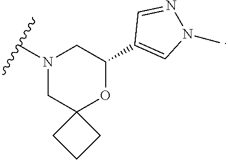 [SMILES: *N1C[C@H](c2cnn(C)c2)OC2(CCC2)C1]

In some embodiments, R²³ is

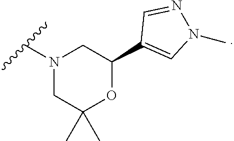 [SMILES: *N1C[C@@H](c2cnn(C)c2)OC(C)(C)C1]

In some embodiments, R²³ is

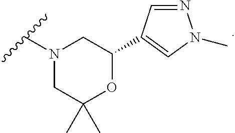 [SMILES: *N1C[C@H](c2cnn(C)c2)OC(C)(C)C1]

In some embodiments, R²³ is

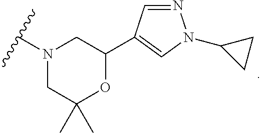 [SMILES: *N1CC(c2cnn(C3CC3)c2)OC(C)(C)C1]

In some embodiments, R²³ is

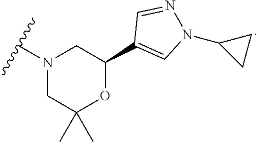 [SMILES: *N1C[C@@H](c2cnn(C3CC3)c2)OC(C)(C)C1]

In some embodiments, R²³ is

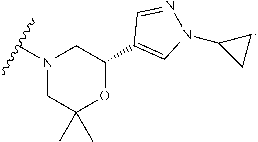 [SMILES: *N1C[C@H](c2cnn(C3CC3)c2)OC(C)(C)C1]

In some embodiments, R²³ is

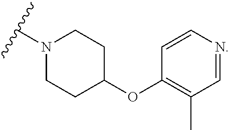 [SMILES: *N1CCC(Oc2ccncc2C)CC1]

In some embodiments, R²³ is

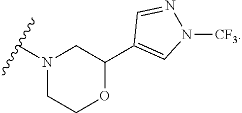 [SMILES: *N1CCOC(c2cnn(C(F)(F)F)c2)C1]

In some embodiments, R²³ is

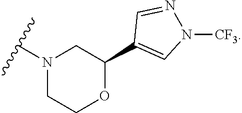 [SMILES: *N1CCO[C@H](c2cnn(C(F)(F)F)c2)C1]

In some embodiments, R²³ is

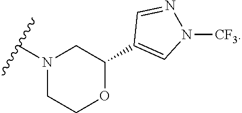 [SMILES: *N1CCO[C@@H](c2cnn(C(F)(F)F)c2)C1]

In some embodiments, R²³ is

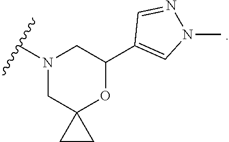 [SMILES: *N1CC(c2cnn(C)c2)OC2(CC2)C1]

In some embodiments, R²³ is

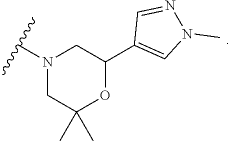 [SMILES: *N1CC(c2cnn(C)c2)OC(C)(C)C1]

In some embodiments, R²³ is

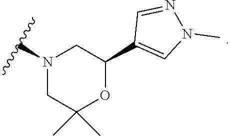

In some embodiments, R²³ is

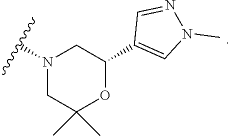

In some embodiments, R²³ is

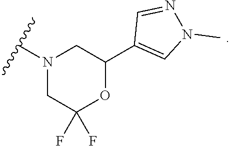 [SMILES: *N1CC(c2cnn(C)c2)OC(F)(F)C1]

In some embodiments, R²³ is

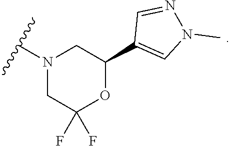 [SMILES: *N1C[C@@H](c2cnn(C)c2)OC(F)(F)C1]

In some embodiments, R²³ is

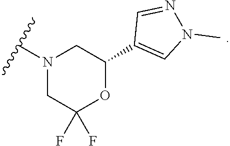 [SMILES: *N1C[C@H](c2cnn(C)c2)OC(F)(F)C1]

In some embodiments, R²³ is

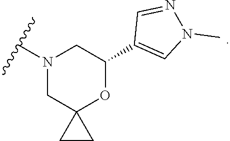 [SMILES: *N1C[C@H](c2cnn(C)c2)OC2(CC2)C1]

In some embodiments, R²³ is

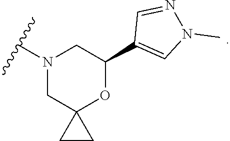 [SMILES: *N1C[C@@H](c2cnn(C)c2)OC2(CC2)C1]

In some embodiments, R²³ is

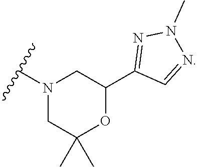 [SMILES: *N1CC(c2cnn(C)n2)OC(C)(C)C1]

In some embodiments, R²³ is

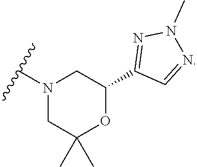 [SMILES: *N1C[C@H](c2cnn(C)n2)OC(C)(C)C1]

In some embodiments, R²³ is

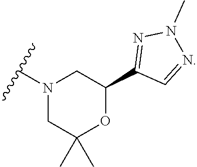 [SMILES: *N1C[C@@H](c2cnn(C)n2)OC(C)(C)C1]

In some embodiments, R²³ is

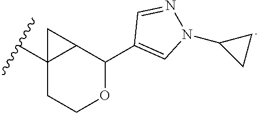 [SMILES: *C12CCOC(c3cnn(C4CC4)c3)C1C2]

In some embodiments, R²³ is

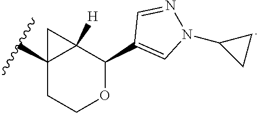 [SMILES: *[C@]12CCO[C@H](c3cnn(C4CC4)c3)[C@H]1C2]

In some embodiments, R²³ is

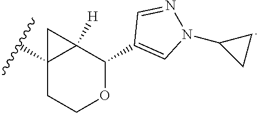 [SMILES: *[C@@]12CCO[C@@H](c3cnn(C4CC4)c3)[C@@H]1C2]

In some embodiments, R²³ is

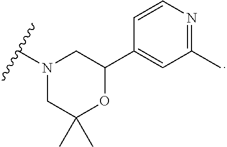 [SMILES: *N1CC(c2ccnc(C)c2)OC(C)(C)C1]

In some embodiments, R²³ is

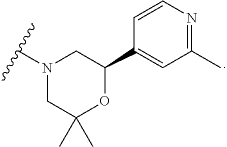 [SMILES: *N1C[C@@H](c2ccnc(C)c2)OC(C)(C)C1]

In some embodiments, R²³ is

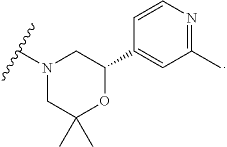 [SMILES: *N1C[C@H](c2ccnc(C)c2)OC(C)(C)C1]

In some embodiments, R²³ is

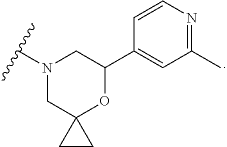 [SMILES: *N1CC(c2ccnc(C)c2)OC2(CC2)C1]

In some embodiments, R²³ is

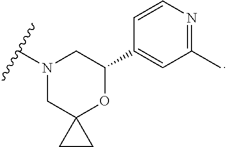 [SMILES: *N1C[C@H](c2ccnc(C)c2)OC2(CC2)C1]

In some embodiments, R²³ is

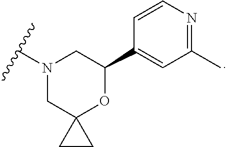 [SMILES: *N1C[C@@H](c2ccnc(C)c2)OC2(CC2)C1]

In some embodiments, R²³ is

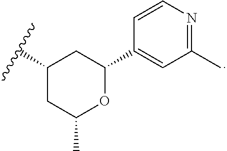 [SMILES: *[C@H]1C[C@@H](C)O[C@@H](c2ccnc(C)c2)C1]

In some embodiments, R²³ is

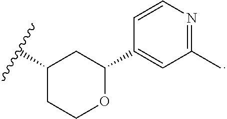 [SMILES: *[C@H]1CCO[C@@H](c2ccnc(C)c2)C1]

In some embodiments, R²³ is

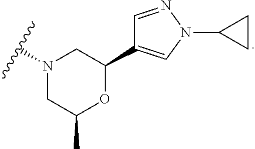

In some embodiments, R²³ is

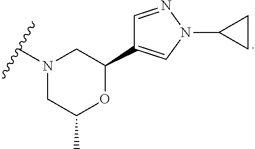 [SMILES: *N1C[C@@H](C)O[C@H](c2cnn(C3CC3)c2)C1]

In some embodiments, R²³ is

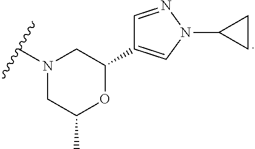 [SMILES: *N1C[C@@H](C)O[C@@H](c2cnn(C3CC3)c2)C1]

In some embodiments, R²³ is

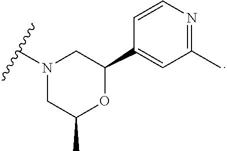 [SMILES: *N1C[C@@H](c2ccnc(C)c2)O[C@@H](C)C1]

In some embodiments, R²³ is

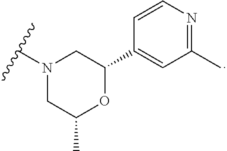 [SMILES: *N1C[C@@H](C)O[C@@H](c2ccnc(C)c2)C1]

In some embodiments, R²³ is

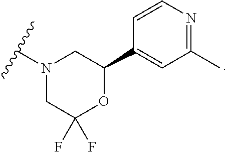 [SMILES: *N1C[C@@H](c2ccnc(C)c2)OC(F)(F)C1]

In some embodiments, R²³ is

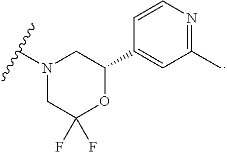 [SMILES: *N1C[C@H](c2ccnc(C)c2)OC(F)(F)C1]

In some embodiments, R²³ is

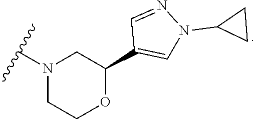 [SMILES: *N1CCO[C@H](c2cnn(C3CC3)c2)C1]

In some embodiments, R²³ is

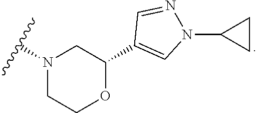

In some embodiments, R²³ is

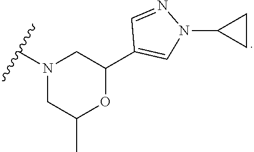 [SMILES: *N1CC(C)OC(c2cnn(C3CC3)c2)C1]

In some embodiments, R²³ is selected from the group consisting of:

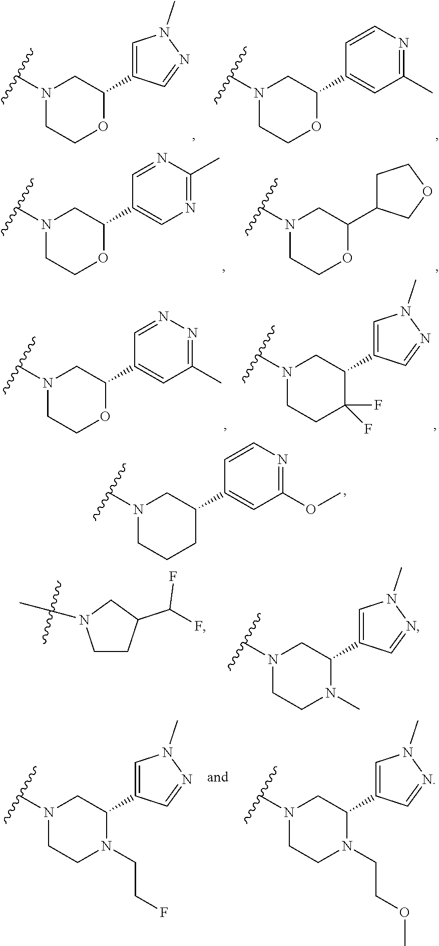

In some embodiments R²³ is selected from the group consisting of,

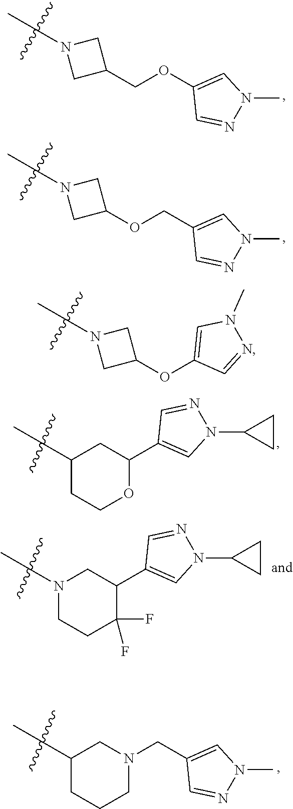

or a pharmaceutically acceptable salt thereof.

In some embodiments, R²³ is of Formula XVI, wherein X³ is N, X⁴ is 0, R⁴ is H, R⁶ is H, X¹⁰ is C(H)(R⁷), R⁷ is H and n is 1. In some embodiments, R²³ is of Formula XVI, wherein X³ is OH, X⁴ is O, R⁴ is H, R⁶ is H, X¹⁰ is C(H)(R⁷), R⁷ is H and n is 1. In some embodiments, R²³ is of Formula XVI, wherein X³ is N, X⁴ is a bond, R⁴ is H, R⁶ is H and n is 0.

In some embodiments, R²³ is of Formula XI:

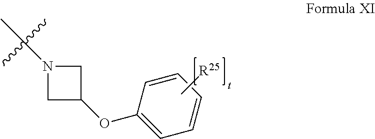

wherein
R²¹ is individually selected from the group consisting of C₁₋₆ alkyl, —O—C₁₋₆ alkyl, halogen, C₁₋₆ haloalkyl and —CN; and
t is 0, 1, 2 or 3.

In some embodiments, R²³ is of Formula XIX:

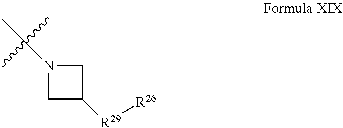

wherein
R²⁶ is a 5- or 6-membered heteroaryl optionally substituted optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and
wherein the C₃₋₆ heterocycloalkyl is optionally substituted with 1 to 3 substituents individually selected from the group consisting of halogen, C₁₋₃ alkyl and —C(O)O(C₁₋₆ alkyl); and
R²⁹ is a bond, —O— or C₁₋₆ hydrocarbon chain wherein one methylene group is optionally replaced with —O—;
or a pharmaceutically acceptable salt thereof.

In some embodiments, R²³ is of Formula XII:

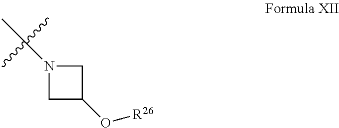

wherein
R²⁶ is a 5- or 6-membered heteroaryl optionally substituted optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and
wherein the C₃₋₆ heterocycloalkyl is optionally substituted with 1 to 3 substituents individually selected from the group consisting of halogen, C₁₋₃ alkyl and —C(O)O(C₁₋₆ alkyl), or a pharmaceutically acceptable salt thereof.

In some embodiments, R²⁶ is R⁵. In some embodiments, R²⁶ is 5-membered heteroaryl optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and wherein the C₃₋₆ heterocycloalkyl is optionally substituted with 1 to 3 substituents individually selected from the group consisting of halogen, C₁₋₃ alkyl and —C(O)O(C₁₋₆ alkyl).

In some embodiments, R²⁶ is of Formula VII:

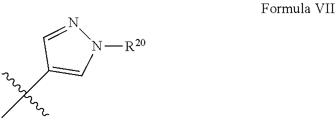

wherein
R²⁰ is C₁₋₃ alkyl, such as —CH₃, or C₃₋₆ cycloalkyl, such as C₃ cycloalkyl.

In some embodiments, R²³ is

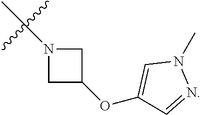 [SMILES: *N1CC(Oc2cnn(C)c2)C1]

In some embodiments, R²³ is of Formula XIV:

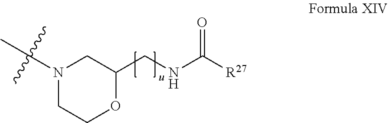

wherein
R²⁷ is C₁₋₆ alkyl; and
u is 1, 2 or 3.

In some embodiments, R²³ is of Formula XVII:

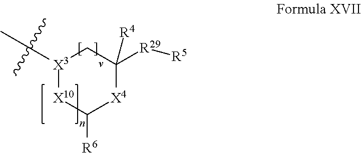

wherein
X³ is N or C(R³);
X⁴ is CH₂, O, CHF, CF₂, NH, NR⁹ or a bond;
X¹⁰ is C(H)(R⁷) or C(O);
X¹¹ is C(R⁴) or N;
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
   (a) the C₃₋₆ cycloalkyl or C₃₋₆ heterocycloalkyl is optionally substituted with 1 to 3 substituents independently selected from oxo (═O), C₁₋₃ alkoxy and halogen;
   (b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₆ alkyl);
(c) the C₁₋₆ alkyl is optionally substituted with 1 to 3 substituents independently selected from —N(H)C(O)R²⁴, -oxo (═O) and —N(R³⁰)(R³¹);
R⁶ is H or C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
n is 0, 1 or 2; and
v is 1 or 2,
or a pharmaceutically acceptable salt thereof.

In some embodiments, v is 0. In some embodiments, v is 2. In some embodiments, v is 1. In some embodiments, X¹¹ is C(R⁴) In some embodiments, R²³ is of Formula XVIII:

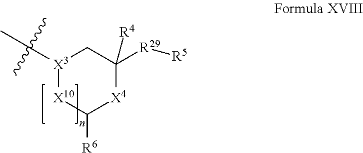

wherein X³, X⁴, X¹⁰, R⁴, R⁵, R⁶, R²⁹ and n are as defined herein, or a pharmaceutically acceptable salt thereof.

In some embodiments, R²³ is a 3 membered ring, i.e. X³, (X¹³)v, X¹¹, X⁴, X¹² and (X¹⁰)ₙ together form a 3 membered ring. For example, in some embodiments, R²³ is of Formula XLVI wherein n is 0, v is 0 and X⁴ is a bond. In some embodiments, R²³ is of Formula XLVI wherein X³ is C(R⁸); X⁴ is a bond; n is 0; X¹¹ is C(R⁴); X¹² is C(R⁶)(R^{6b}); and v is 0.

In some embodiments, X³ is C(H), X¹² is C(H)₂, X⁴ is a bond, X¹¹ is C(H), n is 0 and v is 0. In some embodiments, R²³ is of Formula LXII:

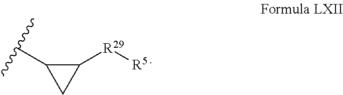

In some embodiments, R²³ is of Formula LXIIa or Formula LXIIb:

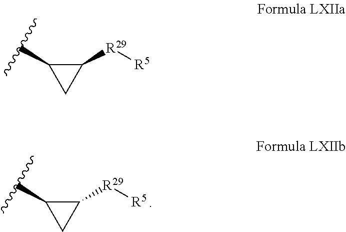

In some embodiments, R²³ is

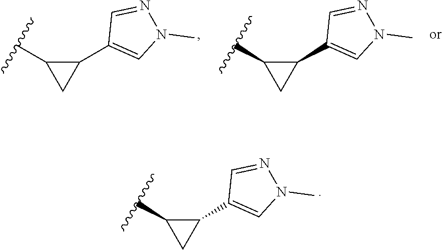

In some embodiments, R²⁹ is a bond, i.e. R²⁹ is absent.

In some embodiments, R²³ is of Formula X:

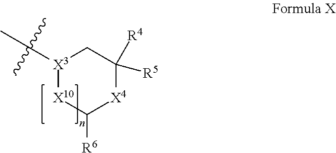

wherein X³, X⁴, X¹⁰, R⁴, R⁵, R⁶ and n are as defined herein, or a pharmaceutically acceptable salt thereof.

In some embodiments, R²⁹ is a C₁₋₃ alkoxy. In some embodiments, R²⁹ is —O—C₁₋₃ alkyl. In some embodiments, R²⁹ is —OCH₂—, —CH₂O—, —CH₂— or O.

In some embodiments, X¹¹ is N. In some embodiments, X¹¹ is C(R⁴). In some embodiments, X¹¹ is C(H). In some embodiments, X¹¹ is C(CH₃).

In some embodiments, n is 0. In some embodiments, n is 1.

In some embodiments, v is 1. In some embodiments, v is 2.

In some embodiments, the compound is of Formula LXI, wherein
X¹ is N;
X² is N;
X¹⁴ is N;
R¹ is —CF₃,
R² is C₁₋₆ alkyl, such as CH₃,
R³ is of Formula IV:

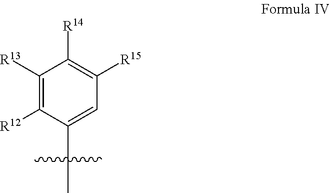

wherein
   R¹² is H, halogen, C₁₋₃ alkyl or C₁₋₃ haloalkyl;
   R¹³ is H, halogen, C₁₋₃ alkyl or C₁₋₃ haloalkyl;
   R¹⁴ is H, halogen, C₁₋₃ alkyl or C₁₋₃ haloalkyl; and
   R¹⁵ is H, halogen, C₁₋₃ alkyl or C₁₋₃ haloalkyl.

In some embodiments, R¹² is halogen, R¹³ is H, R¹⁴ is halogen and R¹⁵ is H. For example, R¹² is F and R¹⁴ is C. In some embodiment, R²³ is of Formula XXXIV:

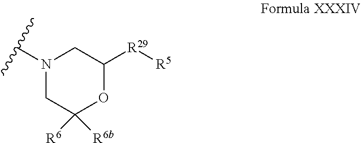

wherein
R⁶ is H, halogen or C₁₋₃ alkyl;
R^{6b} is H, halogen or C₁₋₃ alkyl; and
R²⁹ is a bond.

In some embodiments, R⁶ is H and R^{6b} is H. In some embodiment, R⁵ is of Formula XXII:

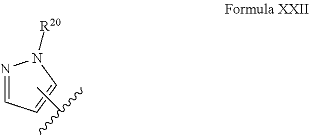

wherein
R²⁰ is C₁₋₃ alkyl or C₃₋₆ cycloalkyl, or a pharmaceutically acceptable salt thereof. In some embodiments, R⁵ is of Formula VII:

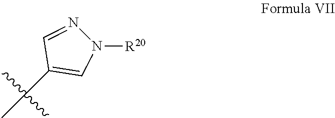

wherein
R²⁰ is C₁₋₃ alkyl or C₃₋₆ cycloalkyl, or a pharmaceutically acceptable salt thereof.

In some embodiments, R²³ is

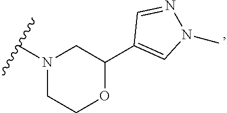 [SMILES: *N1CCOC(c2cnn(C)c2)C1]

such as

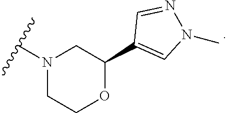 [SMILES: *N1CCO[C@H](c2cnn(C)c2)C1]

In some embodiments, the compound is of Formula LXI, wherein
X¹ is N;
X² is N;
X¹⁴ is N;
R¹ is —CH₃,
R² is C₁₋₆ alkyl, such as CH₃,
R³ is of Formula IV:

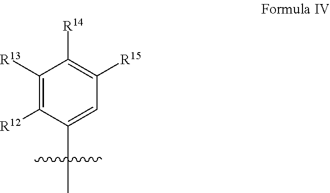

wherein
   R¹² is H, halogen, C₁₋₃ alkyl or C₁₋₃ haloalkyl;
   R¹³ is H, halogen, C₁₋₃ alkyl or C₁₋₃ haloalkyl;
   R¹⁴ is H, halogen, C₁₋₃ alkyl, —CN or C₁₋₃ haloalkyl; and
   R¹⁵ is H, halogen, C₁₋₃ alkyl or C₁₋₃ haloalkyl.

In some embodiments, R¹² is halogen, R¹³ is H, R¹⁴ is haloalkyl and R¹⁵ is H. In some embodiments, R¹² is F and R¹⁴ is CF₃. In some embodiments, R²³ is of Formula XXXIV:

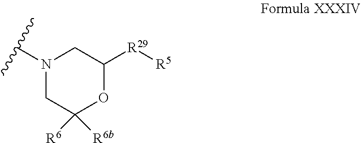

wherein
R⁶ is H, halogen or C₁₋₃ alkyl;
R^{6b} is H, halogen or C₁₋₃ alkyl; and
R²⁹ is a bond,
or a pharmaceutically acceptable salt thereof.

In some embodiments, R⁶ is H and R^{6b} is H. In some embodiments, R⁵ is Formula VIII:

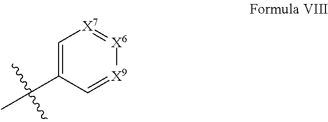

wherein
X⁷ is N or CH;
X³ is N or C(R²¹);
X⁹ is N or C(R²²);
R²¹ is C₁₋₃ alkyl, such as —CH₃; and
R²² is C₁₋₃ alkyl, such as —CH₃.

In some embodiments, R⁵ is

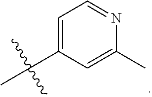 [SMILES: *c1ccnc(C)c1]

In some embodiments, R²³ is

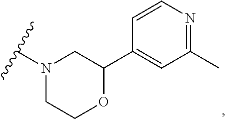 [SMILES: *N1CCOC(c2ccnc(C)c2)C1]

such as

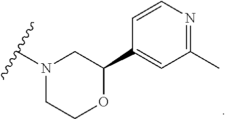 [SMILES: *N1CCO[C@H](c2ccnc(C)c2)C1]

In one aspect, the present invention relates to a compound of Formula LXI:

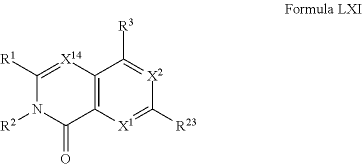

wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
   wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl, C₁₋₃ haloalkyl and CN;
wherein one methylene group of the C₃₋₆ cycloalkyl is optionally replaced with —O— or —N(R²⁸);
   R²⁸ is selected from the group consisting of H, C₁₋₃ alkyl and —C(O)C₁₋₃ alkyl;
   and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl; and
R²³ is selected from the group consisting of:

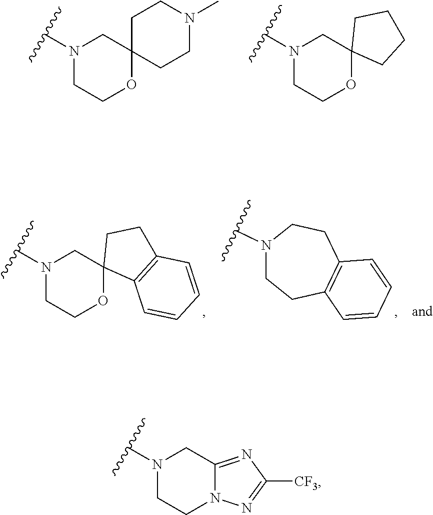 [SMILES: *N1CCOC2(CCN(C)CC2)C1 ; *N1CCOC2(CCCC2)C1 ; *N1CCOC2(CCc3ccccc32)C1 ; *N1CCc2ccccc2CC1 ; *N1CCn2nc(C(F)(F)F)nc2C1]

or a pharmaceutically acceptable salt thereof.

In some embodiments, the compound is selected from the group consisting of:

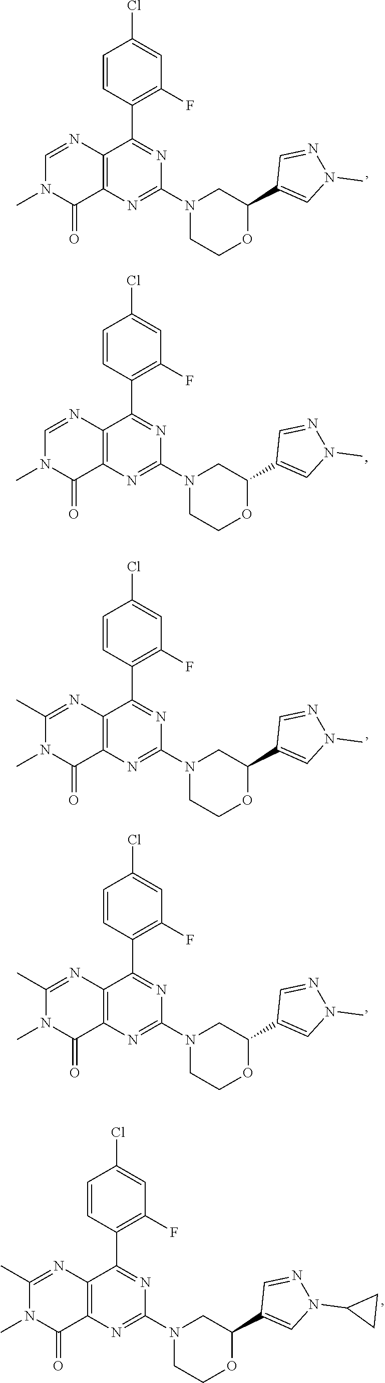

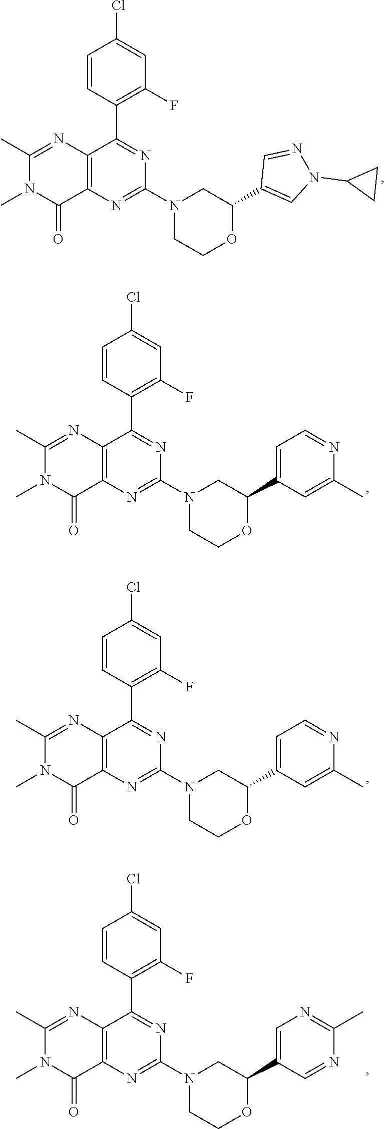

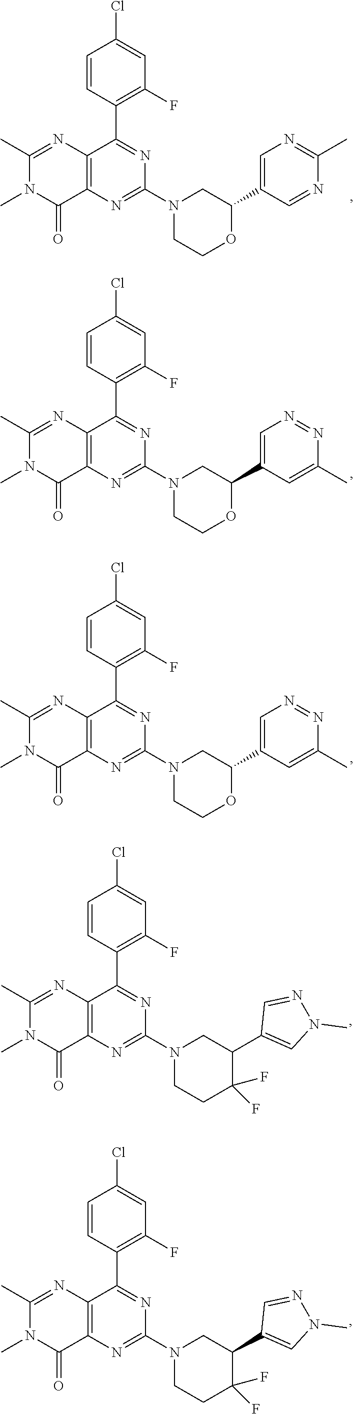

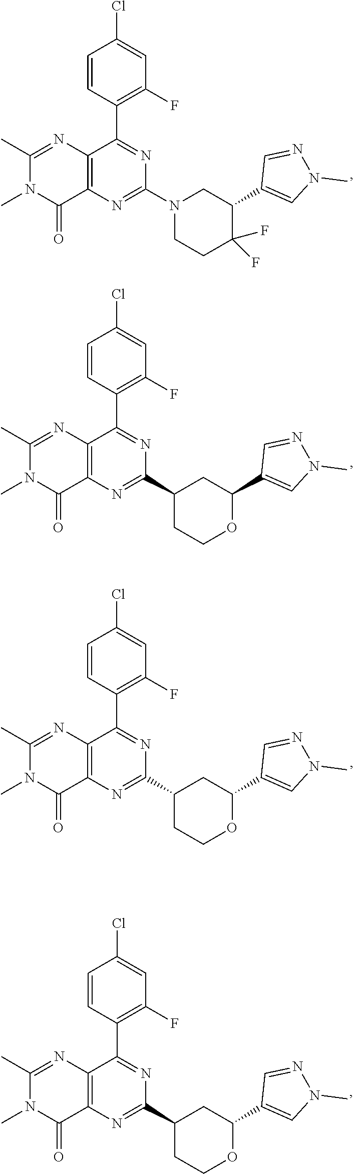

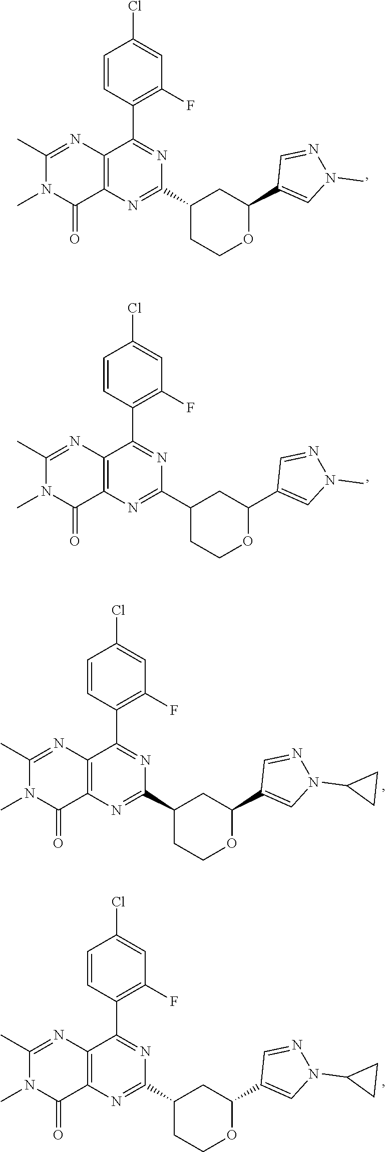

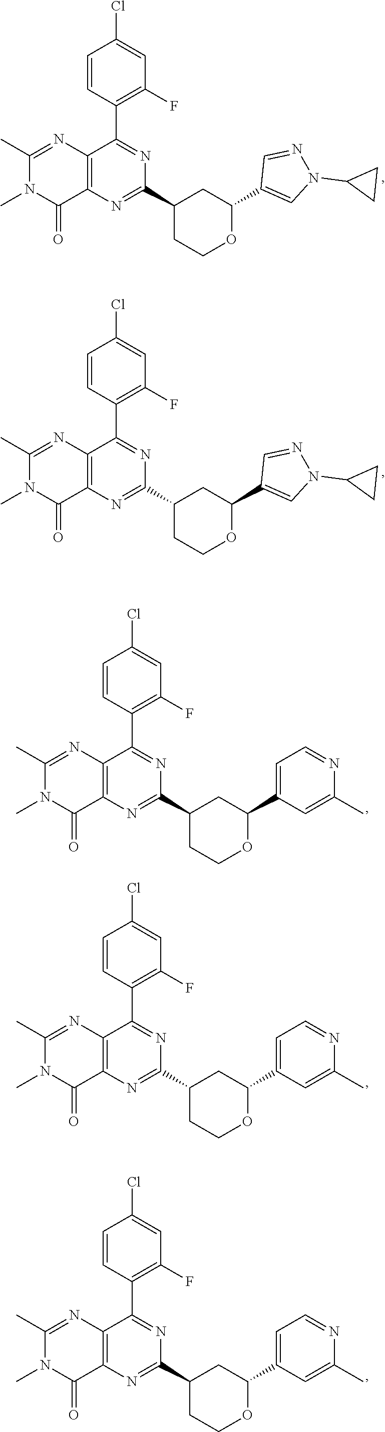

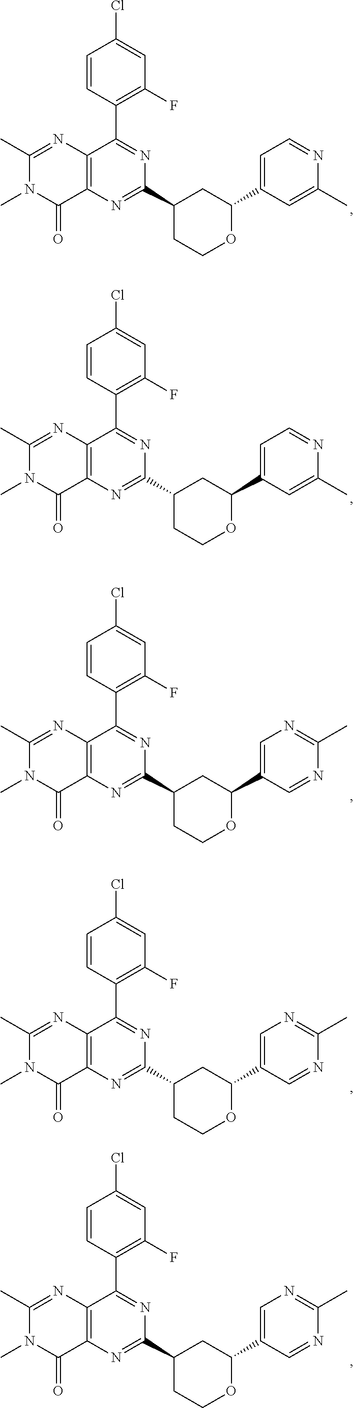

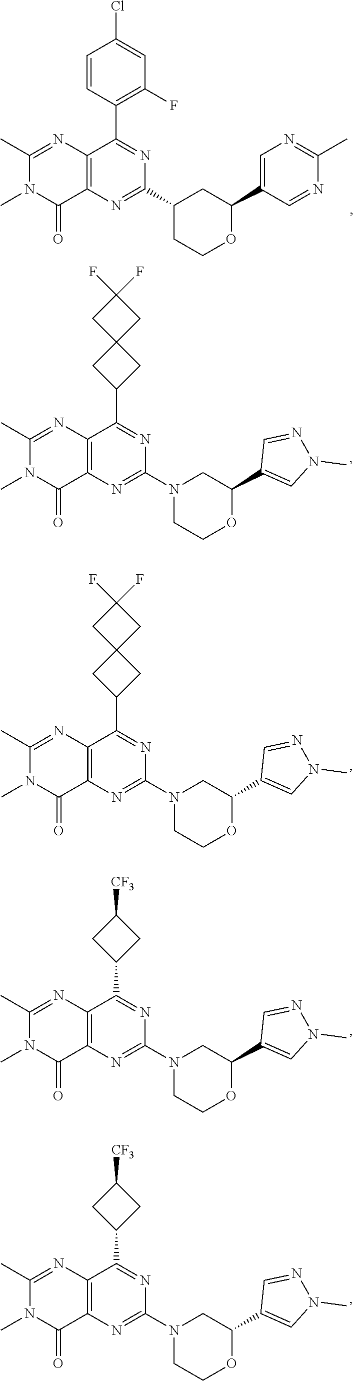

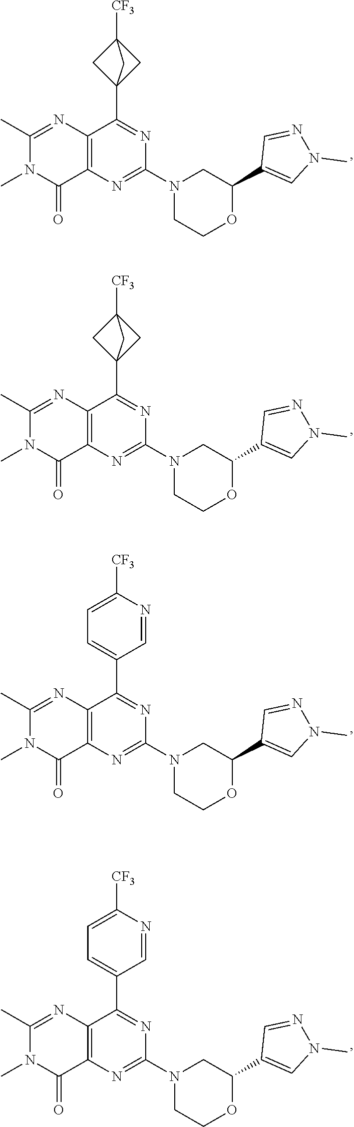

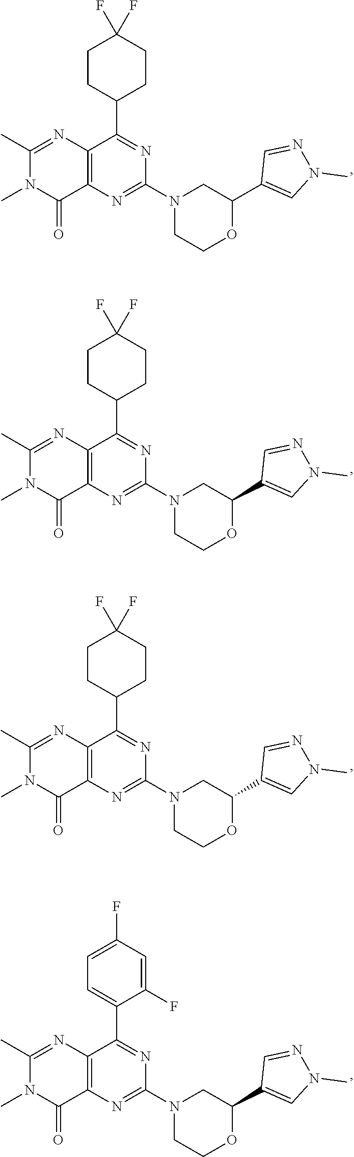

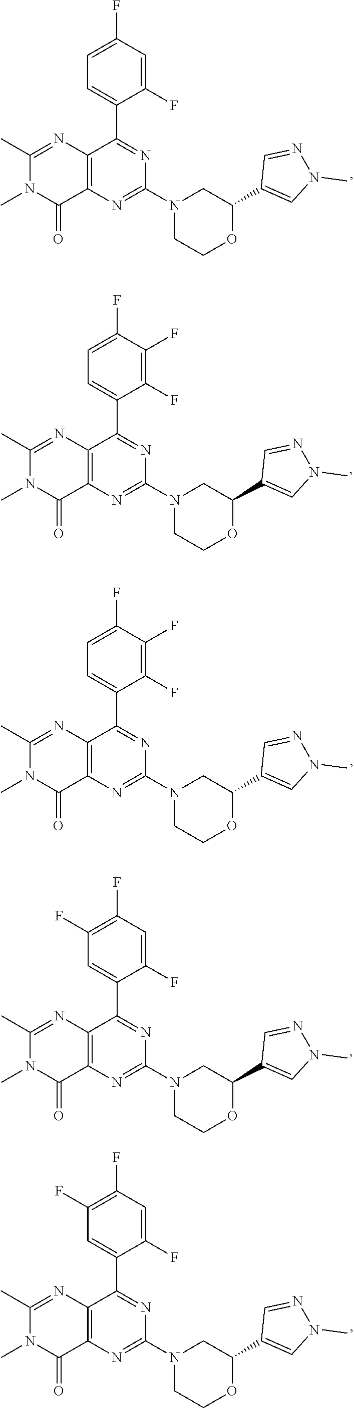

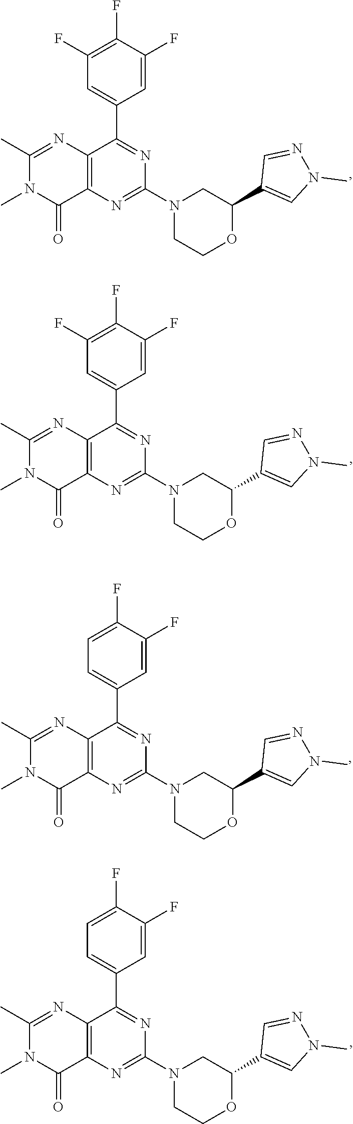

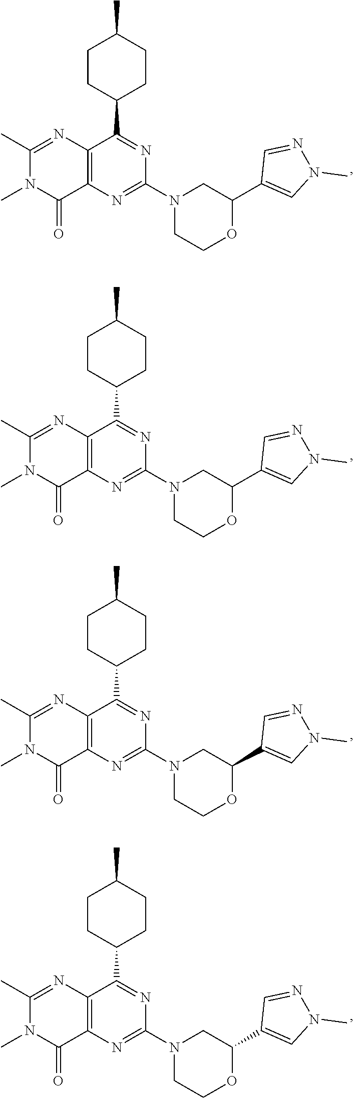

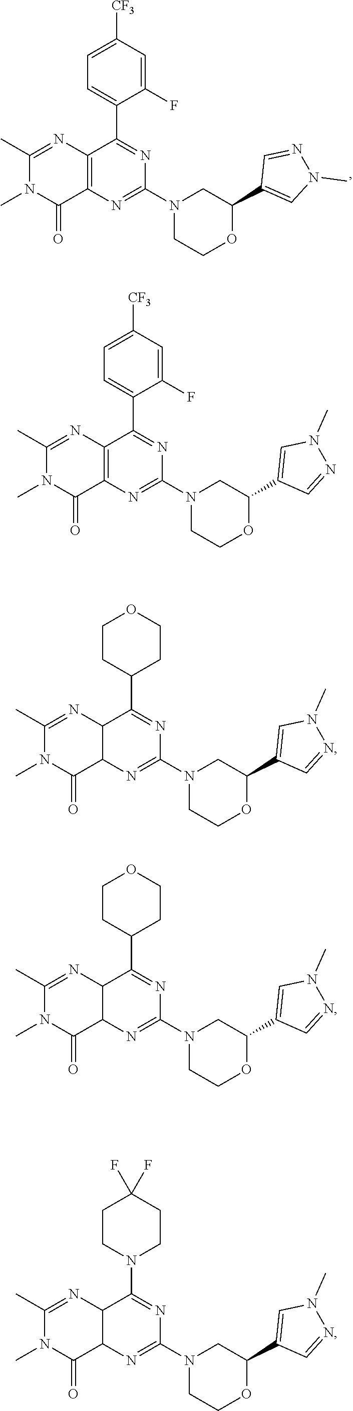

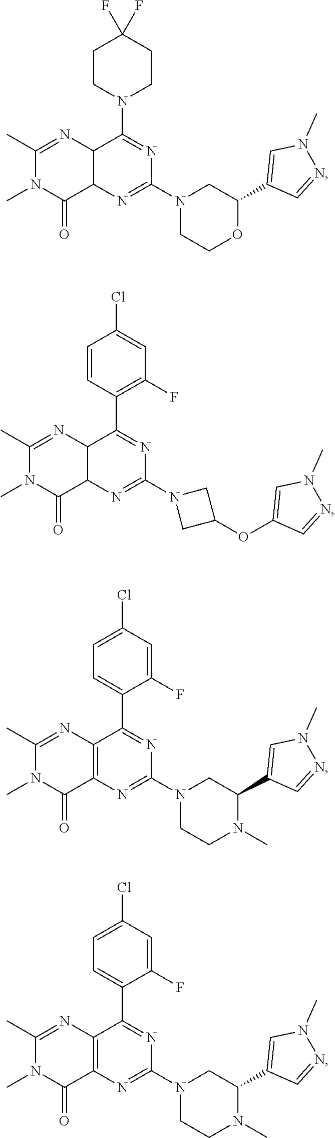 [SMILES: CC1=NC2C(N3CCC(F)(F)CC3)=NC(N3CCO[C@@H](c4cnn(C)c4)C3)=NC2C(=O)N1C ; CC1=NC2C(c3ccc(Cl)cc3F)=NC(N3CC(Oc4cnn(C)c4)C3)=NC2C(=O)N1C ; Cc1nc2c(-c3ccc(Cl)cc3F)nc(N3CCN(C)[C@H](c4cnn(C)c4)C3)nc2c(=O)n1C ; Cc1nc2c(-c3ccc(Cl)cc3F)nc(N3CCN(C)[C@@H](c4cnn(C)c4)C3)nc2c(=O)n1C]

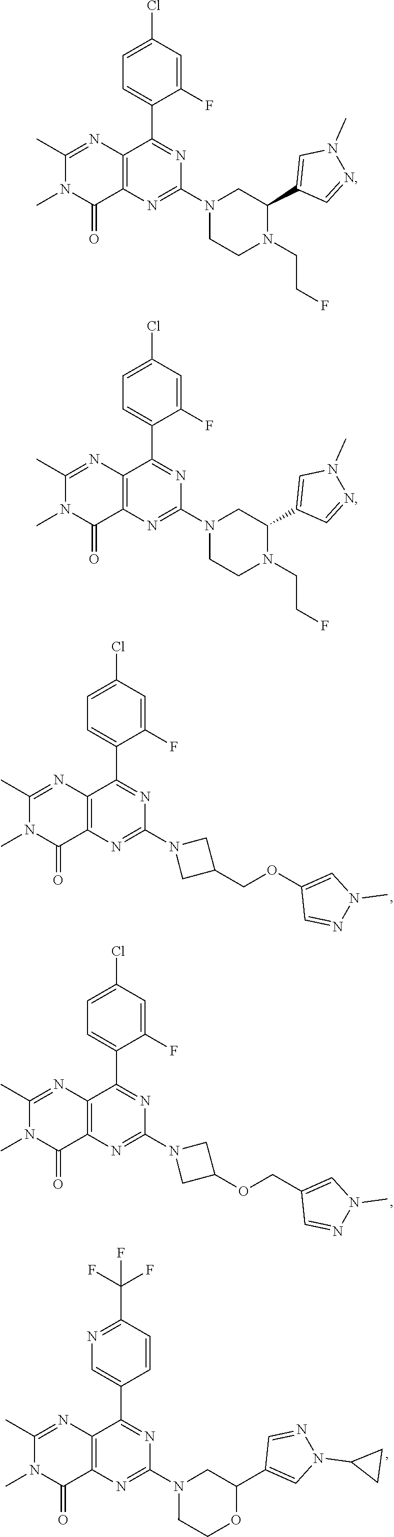

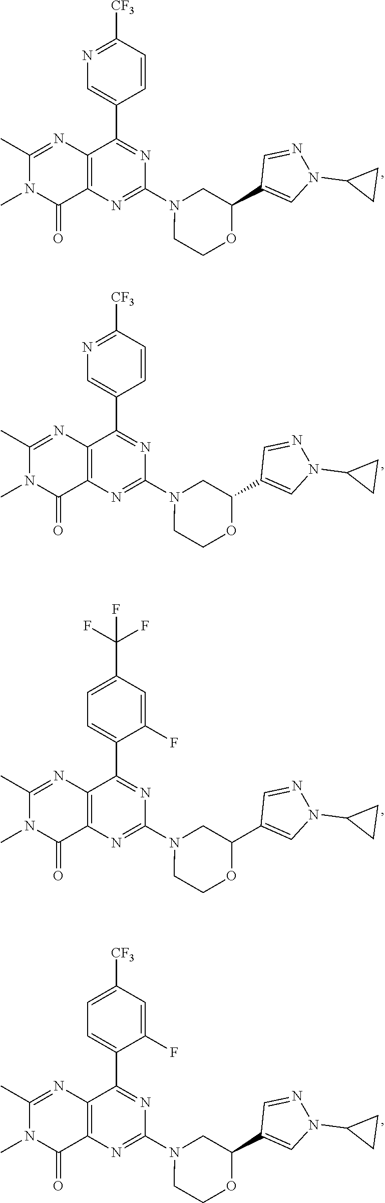 [SMILES: Cc1nc2c(-c3ccc(C(F)(F)F)nc3)nc(N3CCO[C@H](c4cnn(C5CC5)c4)C3)nc2c(=O)n1C ; Cc1nc2c(-c3ccc(C(F)(F)F)nc3)nc(N3CCO[C@@H](c4cnn(C5CC5)c4)C3)nc2c(=O)n1C ; Cc1nc2c(-c3ccc(C(F)(F)F)cc3F)nc(N3CCOC(c4cnn(C5CC5)c4)C3)nc2c(=O)n1C ; Cc1nc2c(-c3ccc(C(F)(F)F)cc3F)nc(N3CCO[C@H](c4cnn(C5CC5)c4)C3)nc2c(=O)n1C]

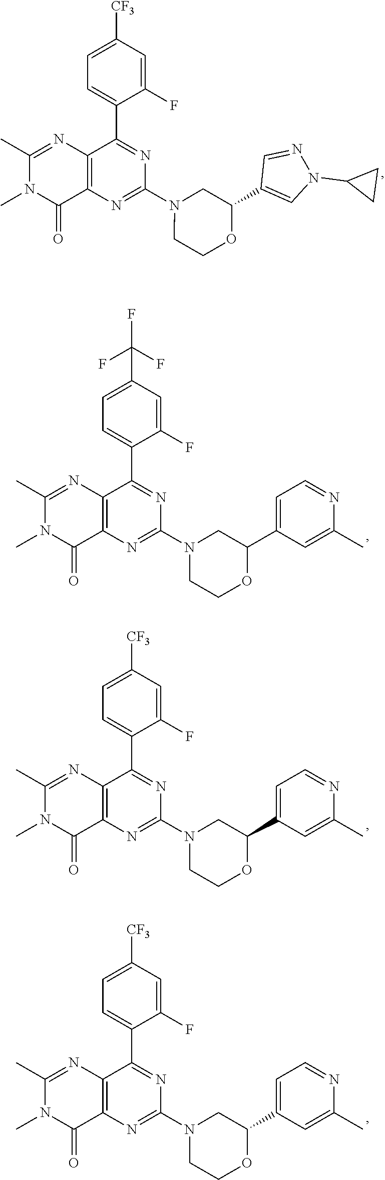

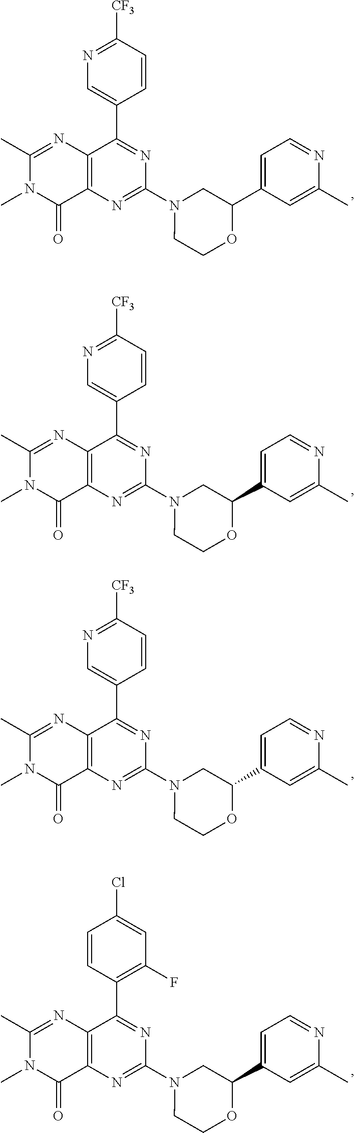

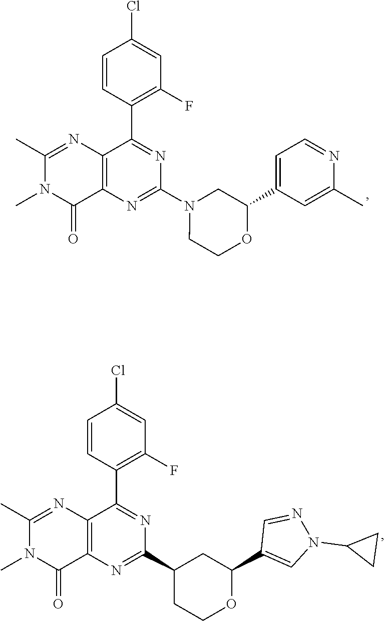

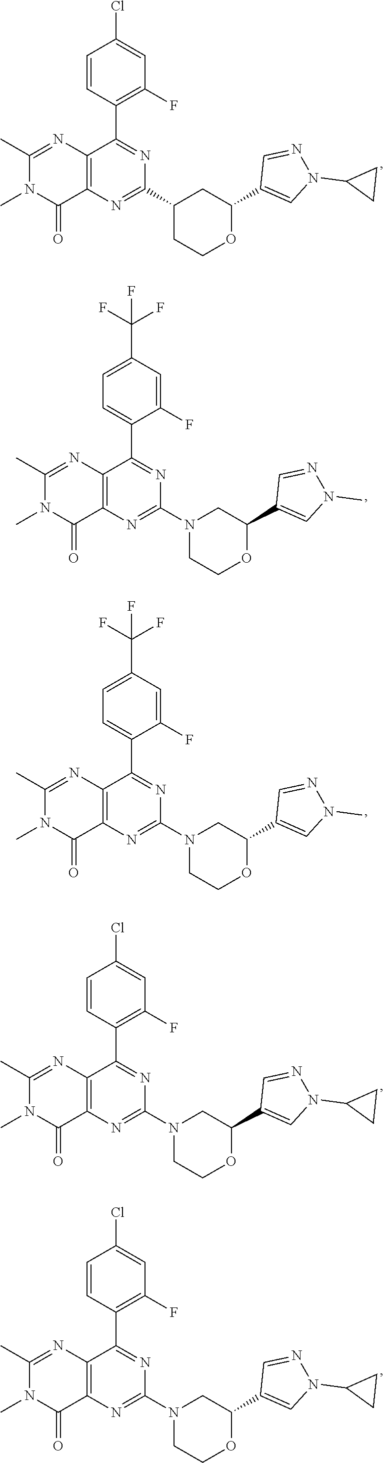

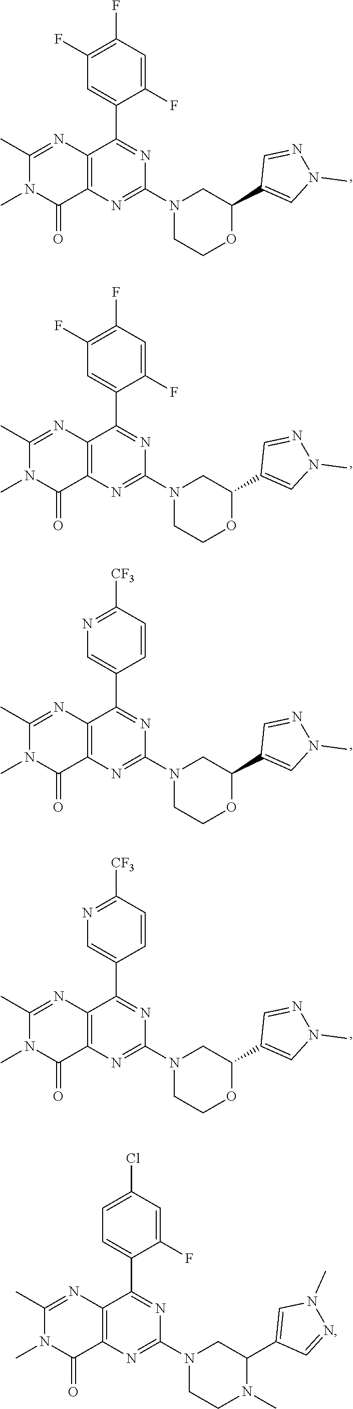

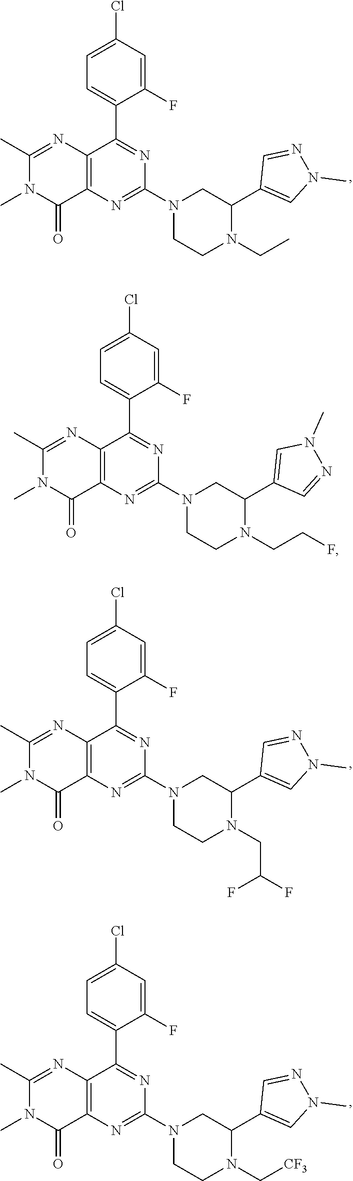

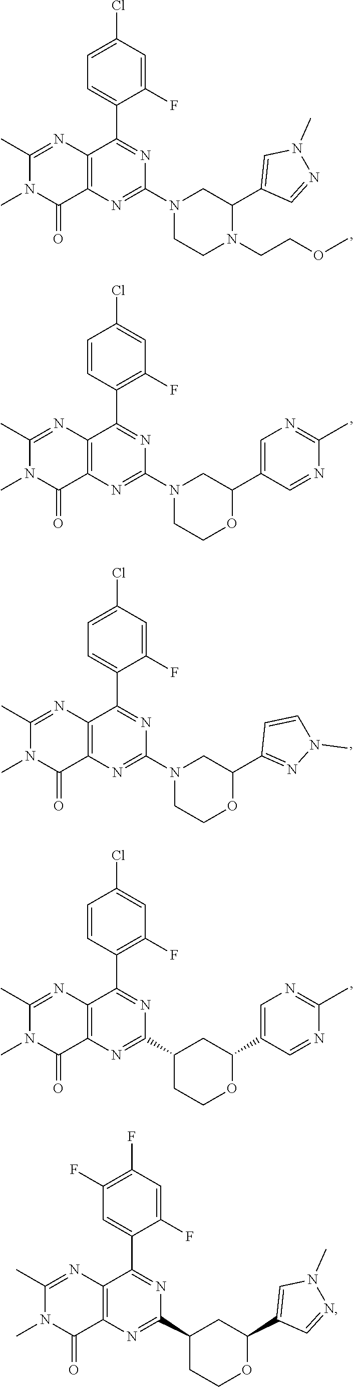

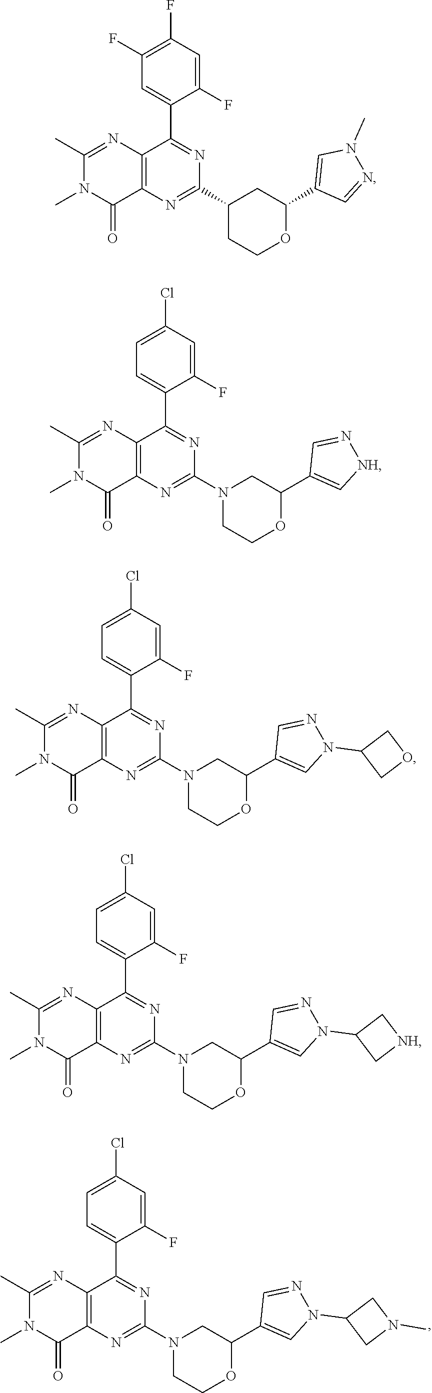

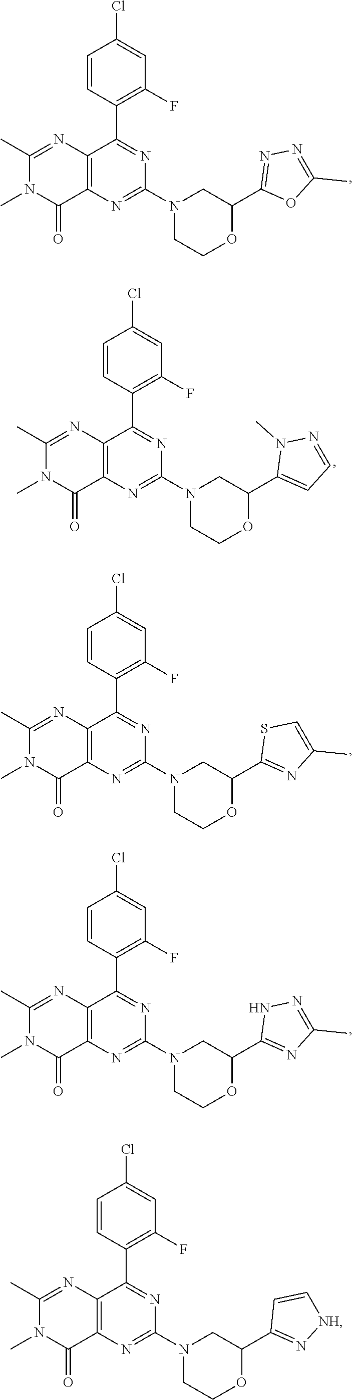

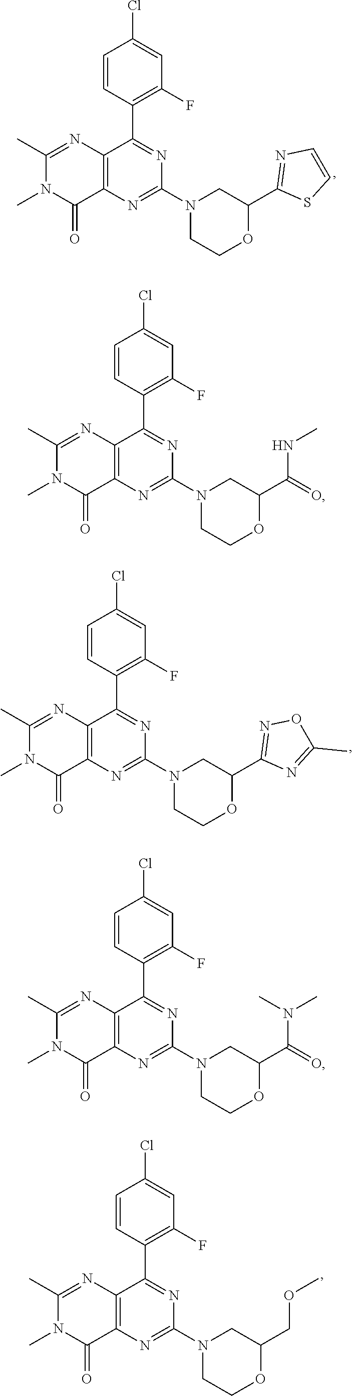

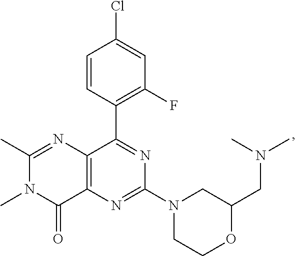

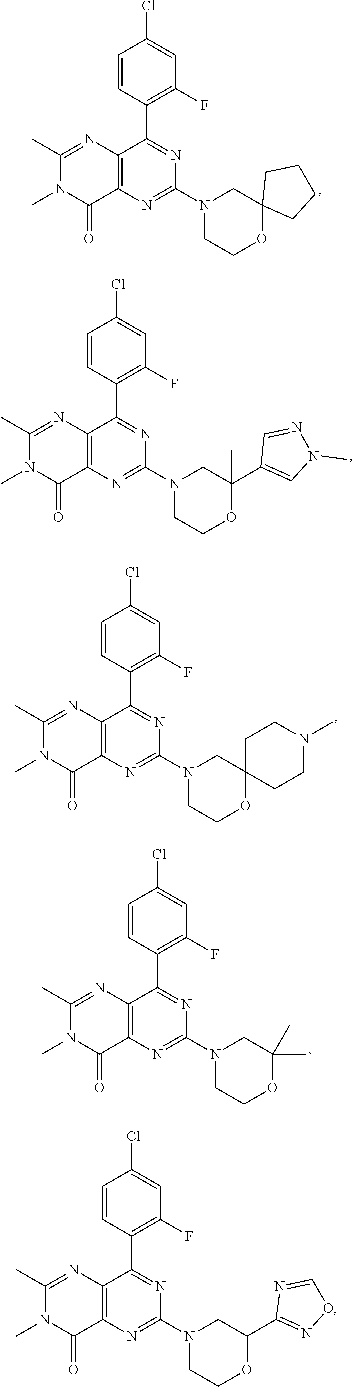

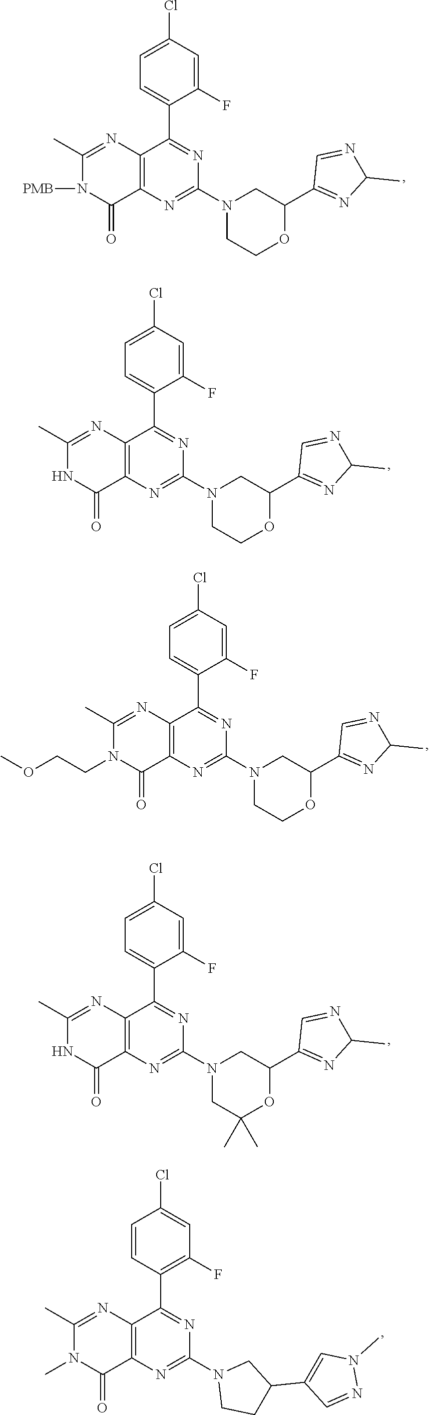

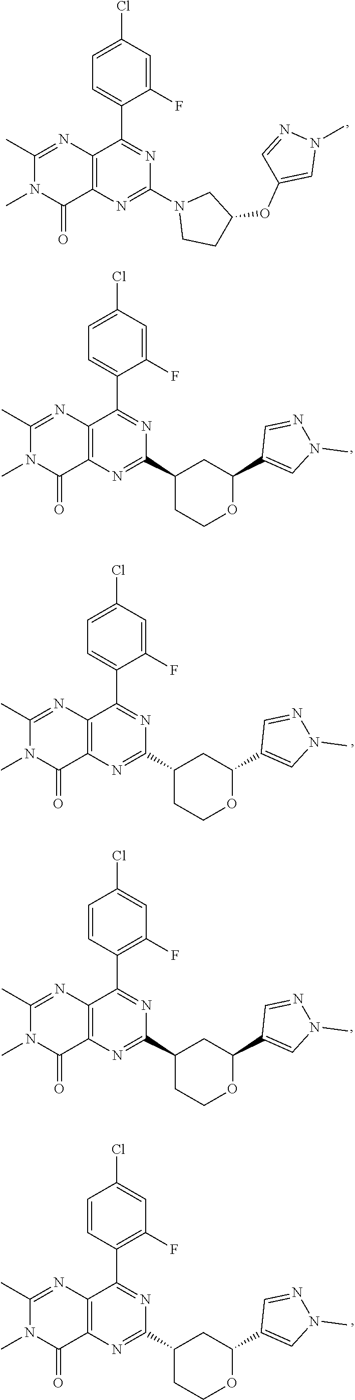

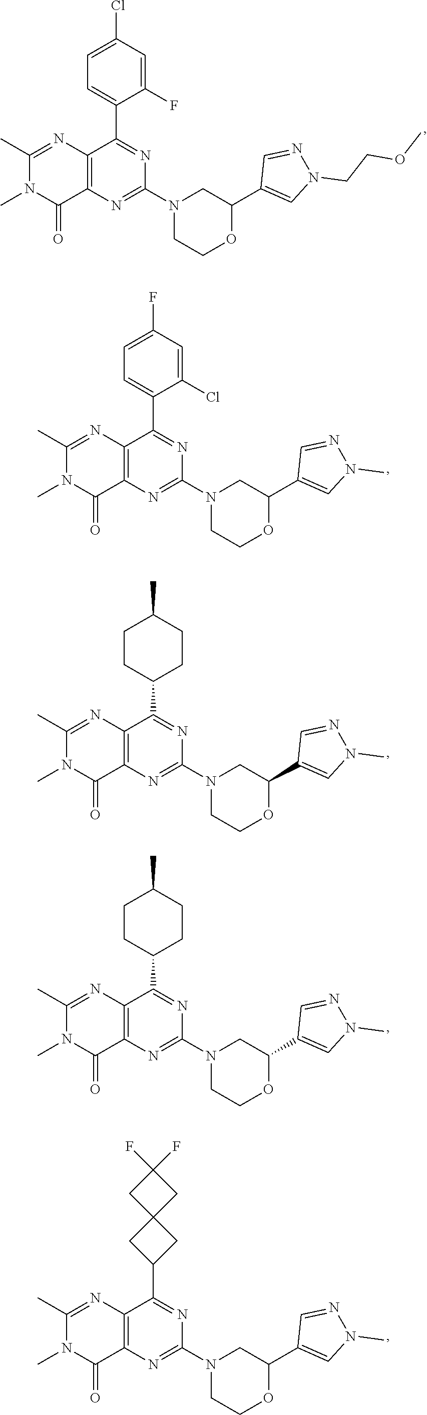

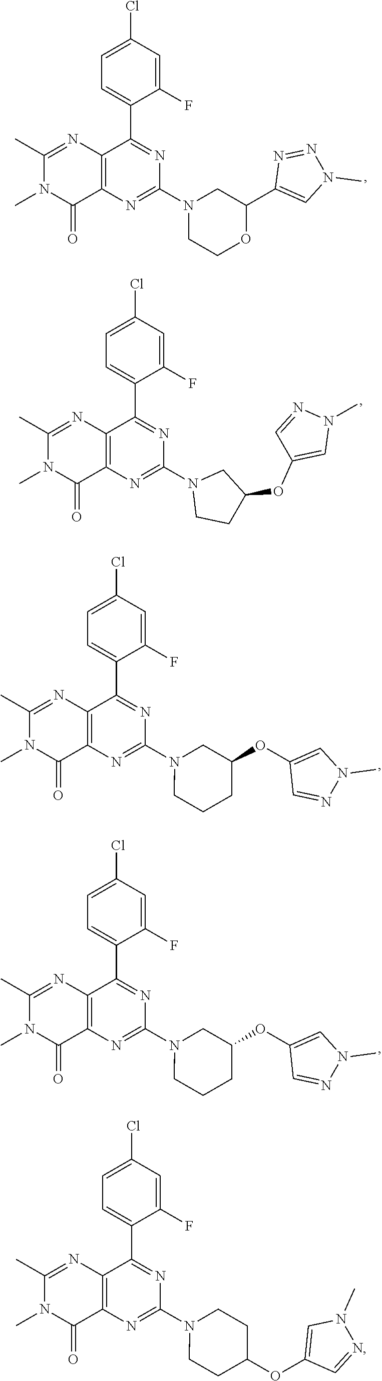

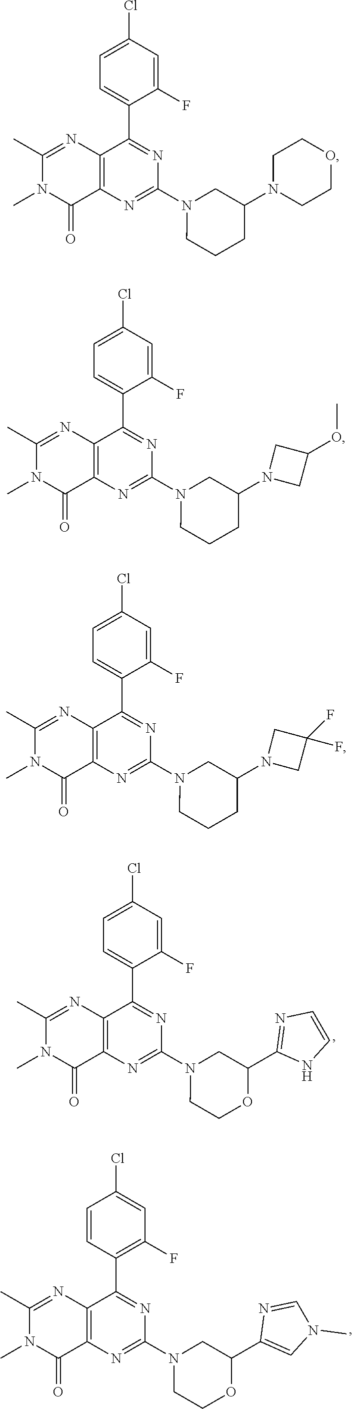

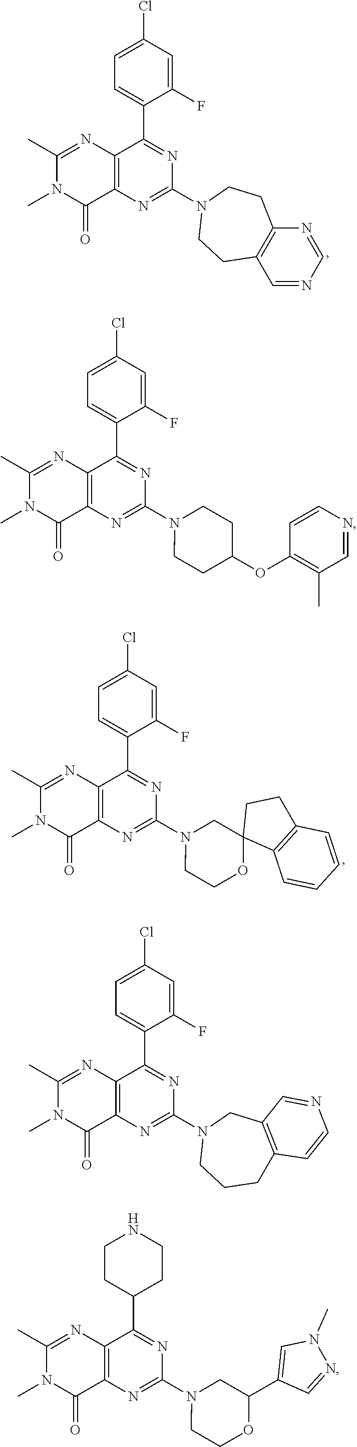 [SMILES: Cc1nc2c(-c3ccc(Cl)cc3F)nc(N3CCc4cncnc4CC3)nc2c(=O)n1C ; Cc1cnccc1OC1CCN(c2nc(-c3ccc(Cl)cc3F)c3nc(C)n(C)c(=O)c3n2)CC1 ; Cc1nc2c(-c3ccc(Cl)cc3F)nc(N3CCOC4(CCc5ccccc54)C3)nc2c(=O)n1C ; Cc1nc2c(-c3ccc(Cl)cc3F)nc(N3CCCc4ccncc4C3)nc2c(=O)n1C ; Cc1nc2c(C3CCNCC3)nc(N3CCOC(c4cnn(C)c4)C3)nc2c(=O)n1C]

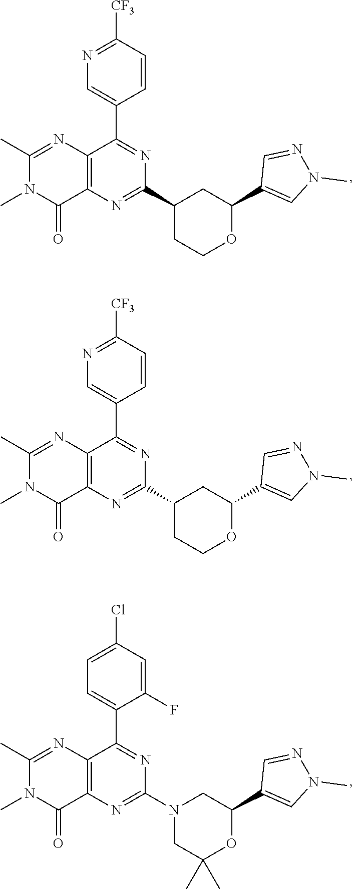

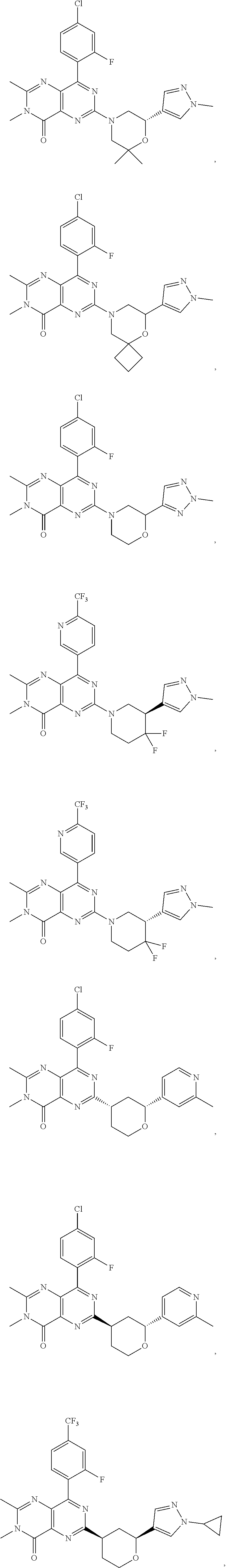

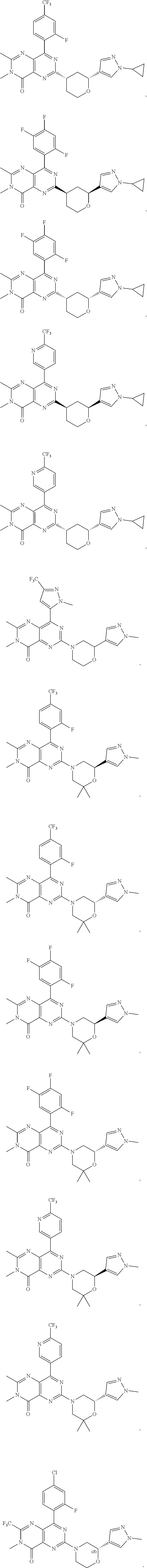

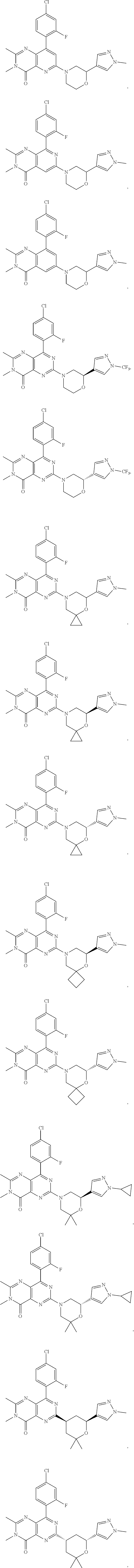

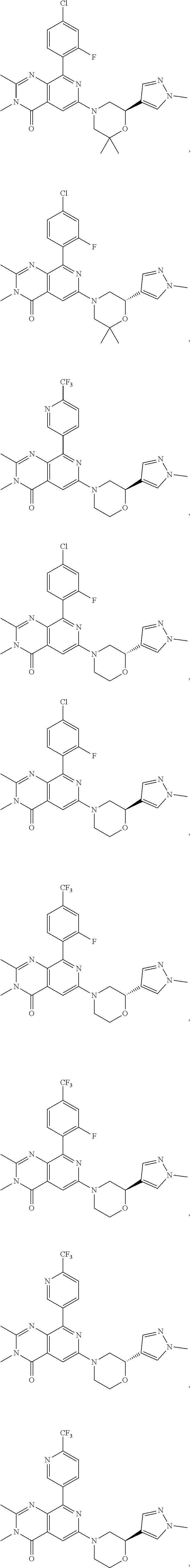

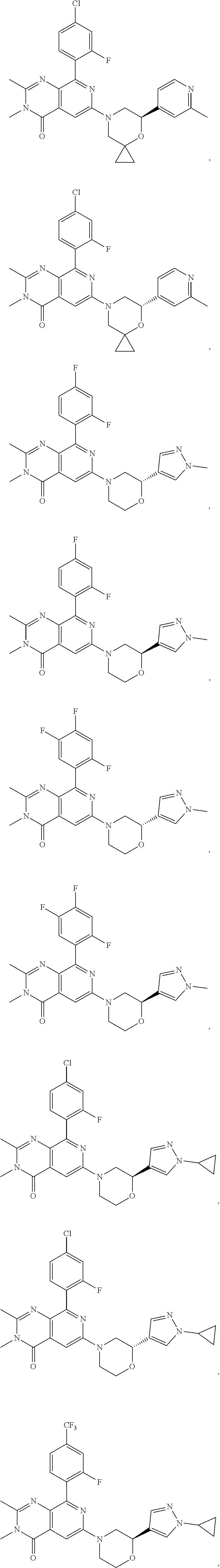

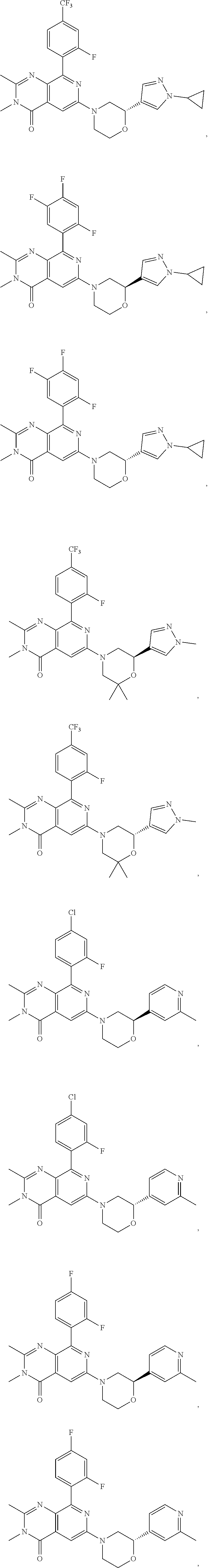

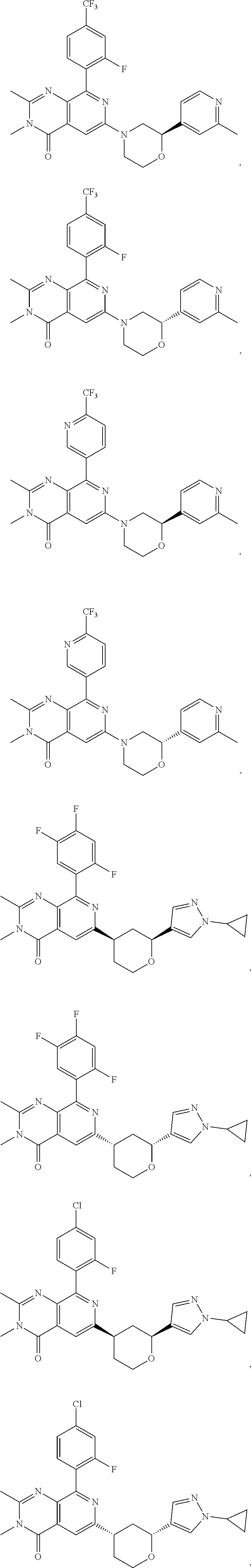

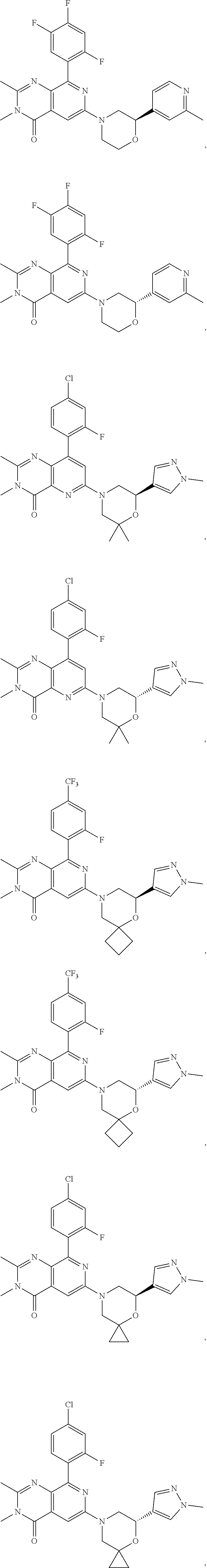

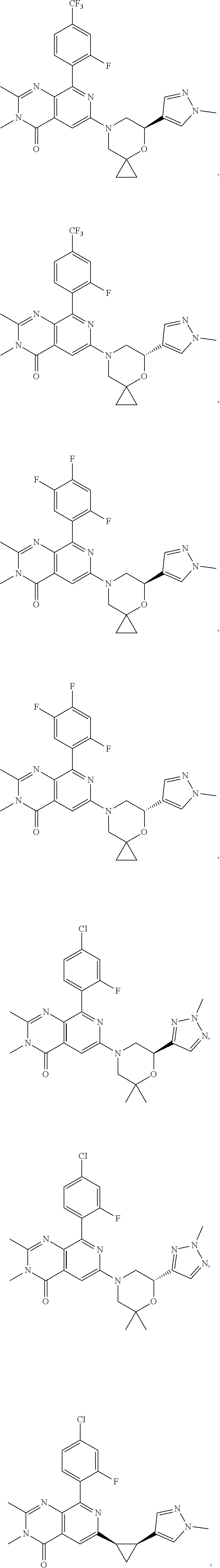

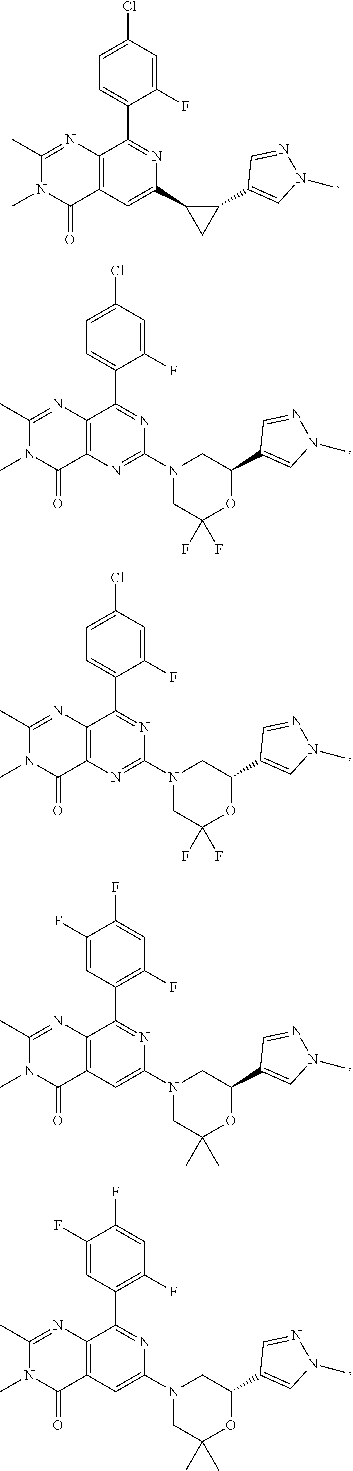

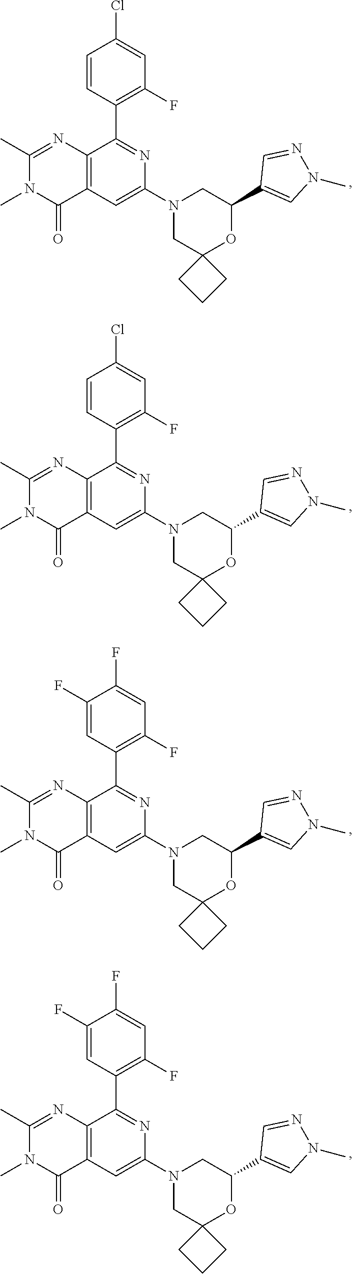

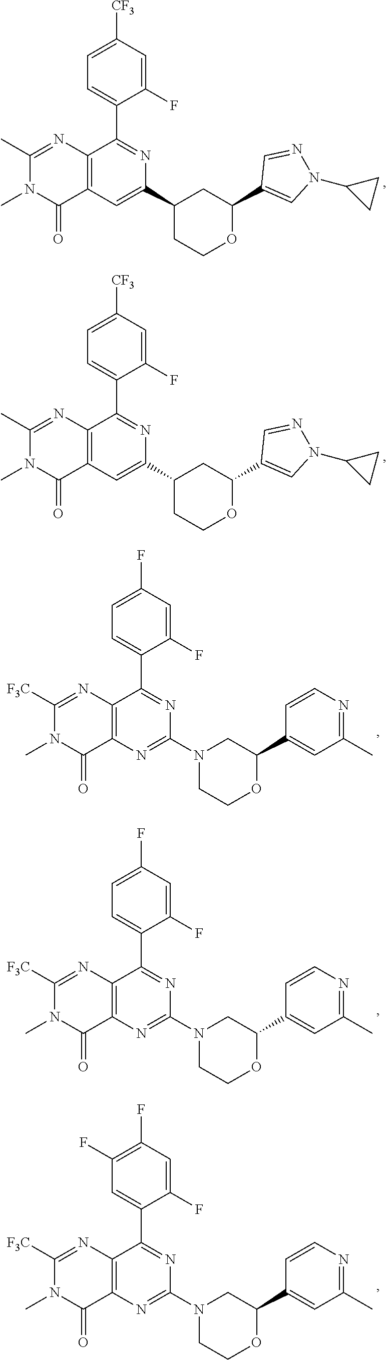 [SMILES: Cc1nc2c(-c3ccc(C(F)(F)F)cc3F)nc([C@@H]3CCO[C@H](c4cnn(C5CC5)c4)C3)cc2c(=O)n1C ; Cc1nc2c(-c3ccc(C(F)(F)F)cc3F)nc([C@H]3CCO[C@@H](c4cnn(C5CC5)c4)C3)cc2c(=O)n1C ; Cc1cc([C@@H]2CN(c3nc(-c4ccc(F)cc4F)c4nc(C(F)(F)F)n(C)c(=O)c4n3)CCO2)ccn1 ; Cc1cc([C@H]2CN(c3nc(-c4ccc(F)cc4F)c4nc(C(F)(F)F)n(C)c(=O)c4n3)CCO2)ccn1 ; Cc1cc([C@@H]2CN(c3nc(-c4cc(F)c(F)cc4F)c4nc(C(F)(F)F)n(C)c(=O)c4n3)CCO2)ccn1]

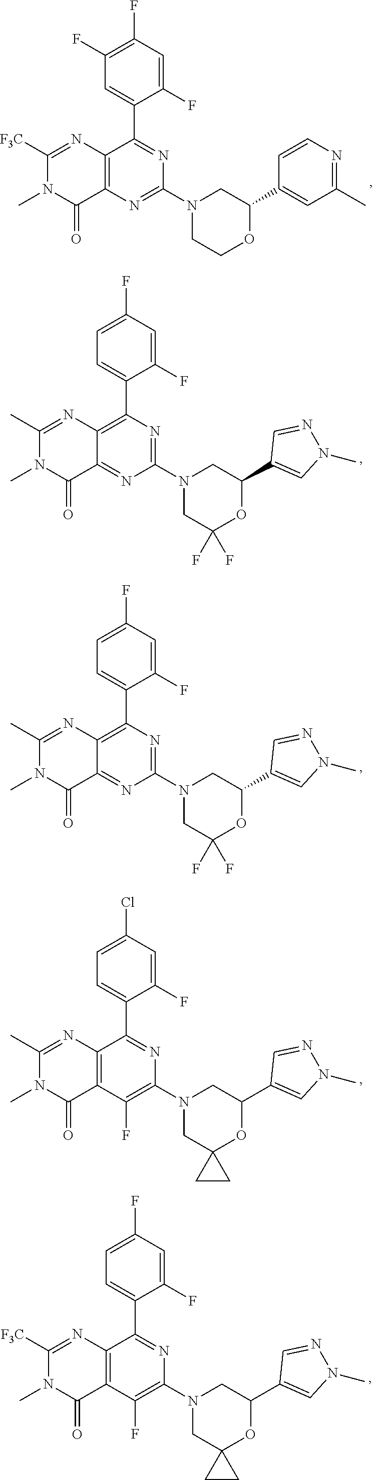

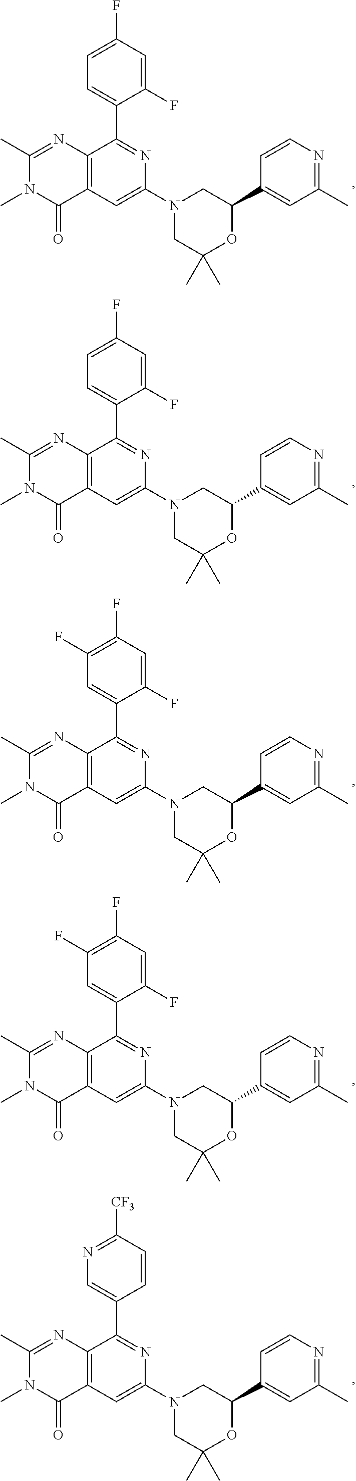

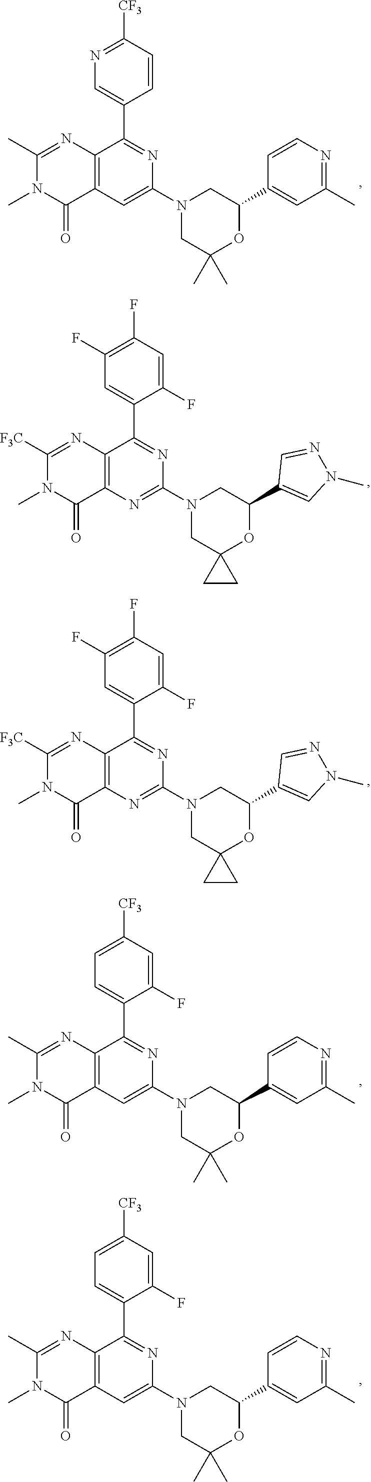

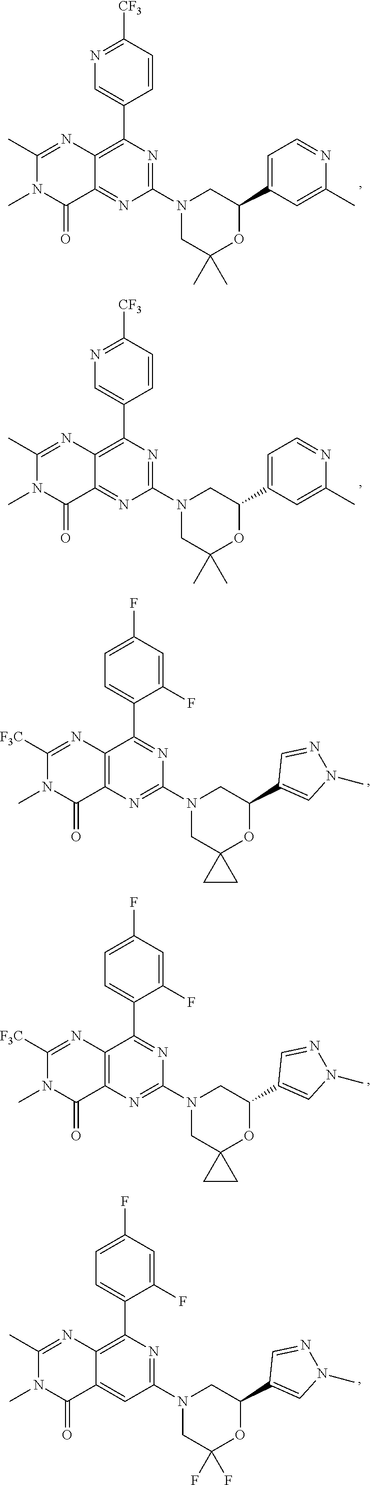

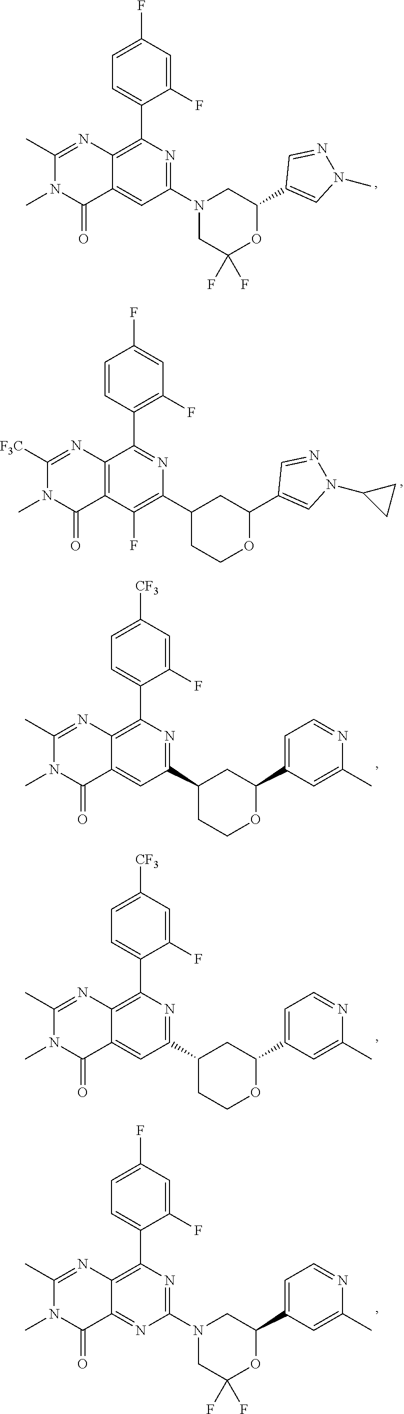

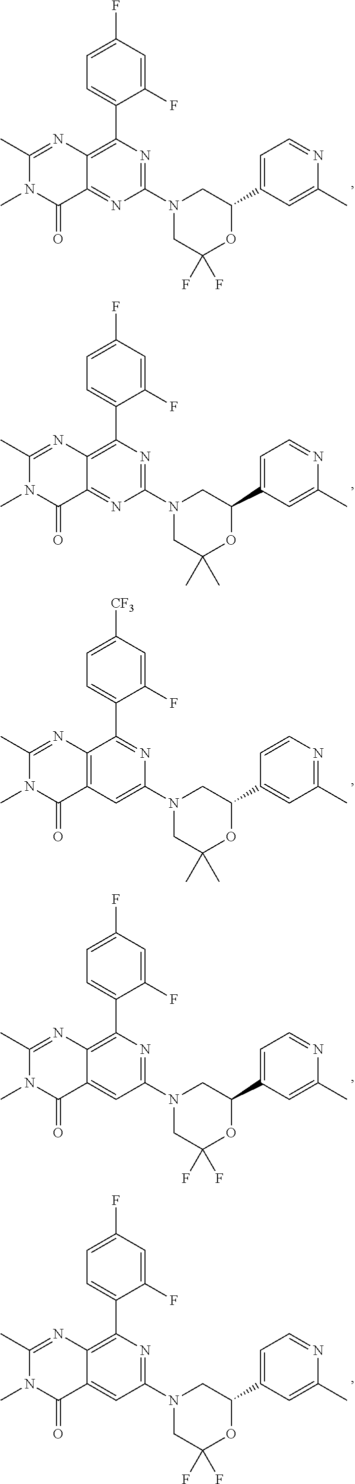

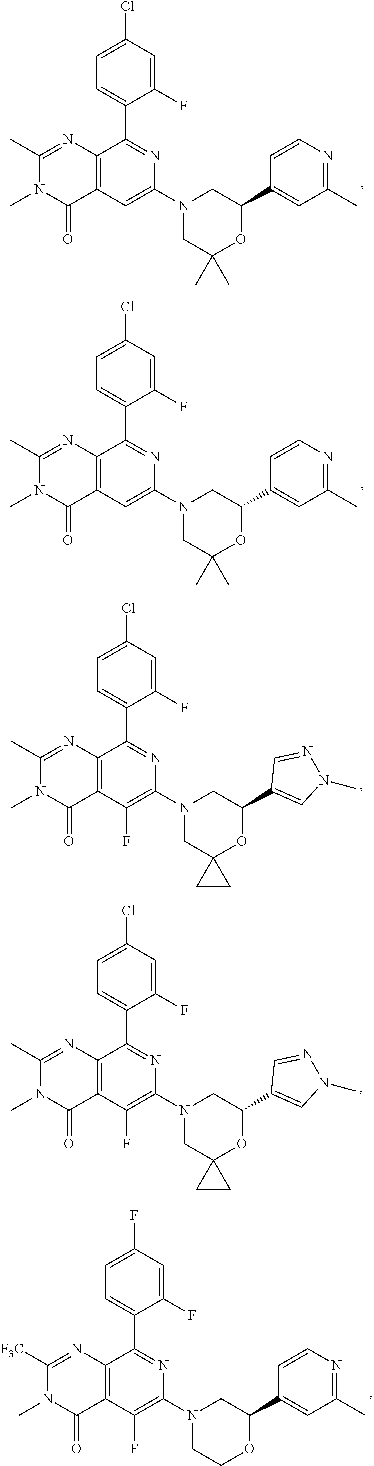

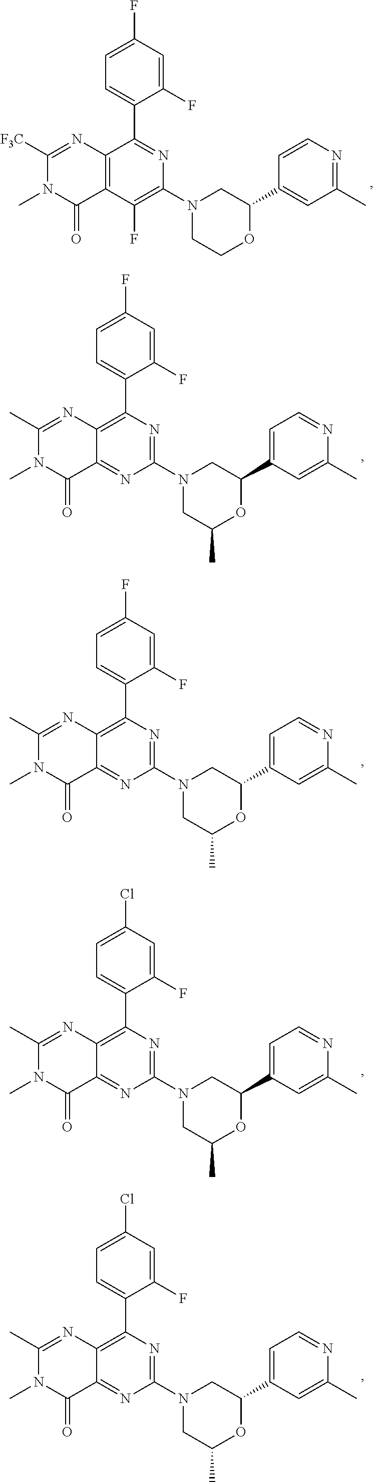

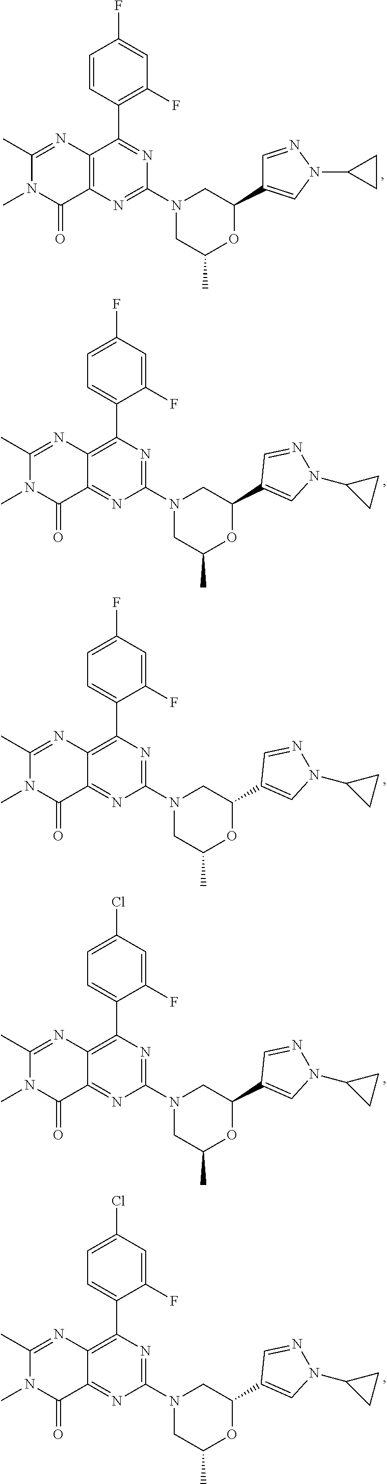 [SMILES: Cc1nc2c(-c3ccc(F)cc3F)nc(N3C[C@@H](C)O[C@H](c4cnn(C5CC5)c4)C3)nc2c(=O)n1C ; Cc1nc2c(-c3ccc(F)cc3F)nc(N3C[C@@H](c4cnn(C5CC5)c4)O[C@@H](C)C3)nc2c(=O)n1C ; Cc1nc2c(-c3ccc(F)cc3F)nc(N3C[C@@H](C)O[C@@H](c4cnn(C5CC5)c4)C3)nc2c(=O)n1C ; Cc1nc2c(-c3ccc(Cl)cc3F)nc(N3C[C@@H](c4cnn(C5CC5)c4)O[C@@H](C)C3)nc2c(=O)n1C ; Cc1nc2c(-c3ccc(Cl)cc3F)nc(N3C[C@@H](C)O[C@@H](c4cnn(C5CC5)c4)C3)nc2c(=O)n1C]

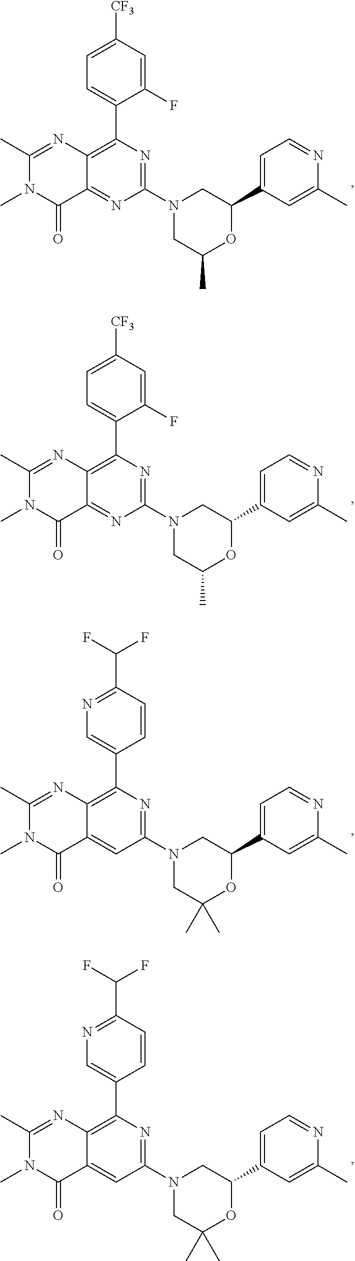

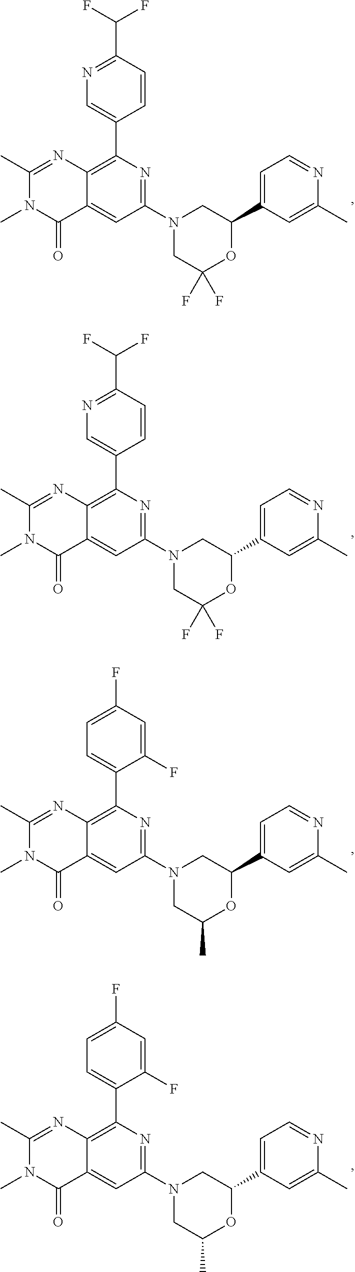

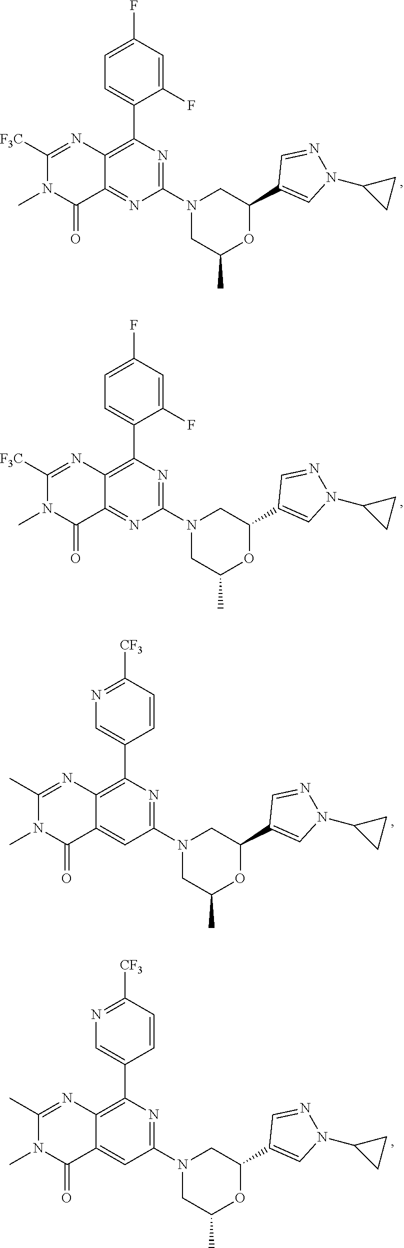 [SMILES: C[C@H]1CN(c2nc(-c3ccc(F)cc3F)c3nc(C(F)(F)F)n(C)c(=O)c3n2)C[C@@H](c2cnn(C3CC3)c2)O1 ; C[C@@H]1CN(c2nc(-c3ccc(F)cc3F)c3nc(C(F)(F)F)n(C)c(=O)c3n2)C[C@H](c2cnn(C3CC3)c2)O1 ; Cc1nc2c(-c3ccc(C(F)(F)F)nc3)nc(N3C[C@@H](c4cnn(C5CC5)c4)O[C@@H](C)C3)cc2c(=O)n1C ; Cc1nc2c(-c3ccc(C(F)(F)F)nc3)nc(N3C[C@@H](C)O[C@@H](c4cnn(C5CC5)c4)C3)cc2c(=O)n1C]

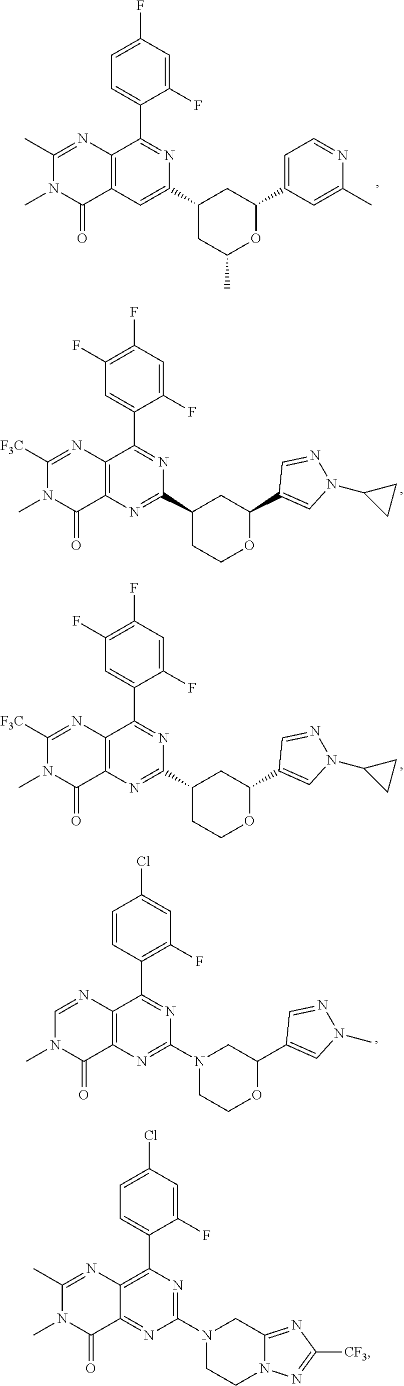

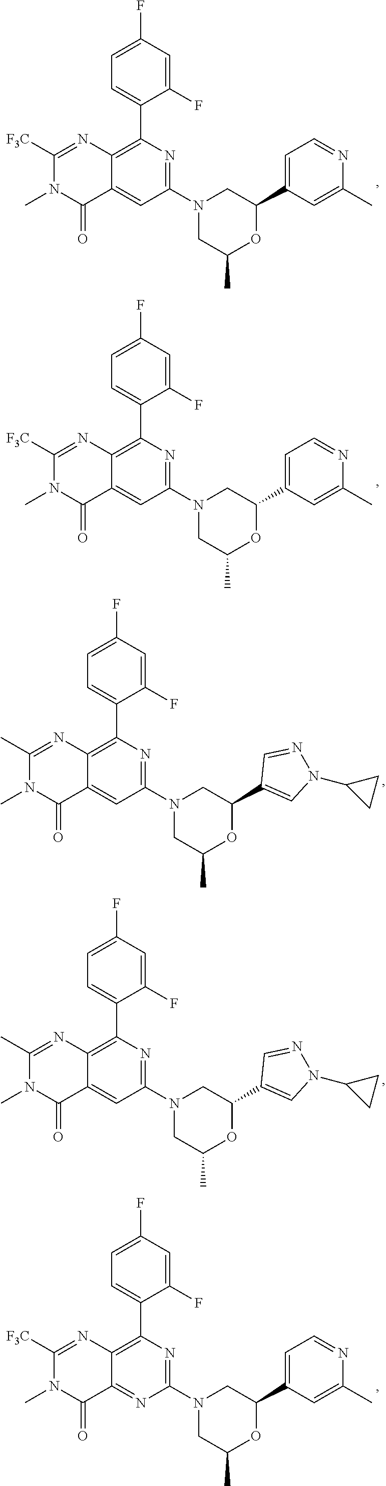

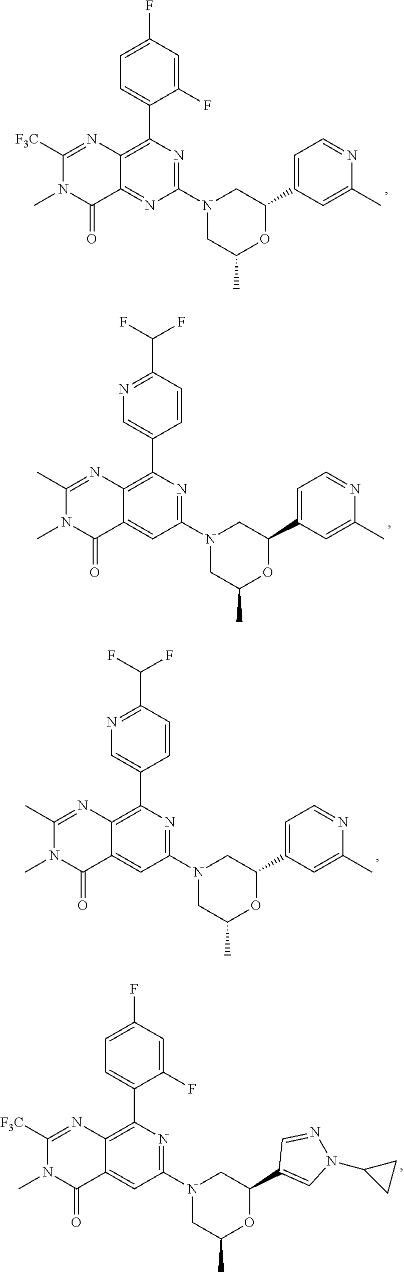

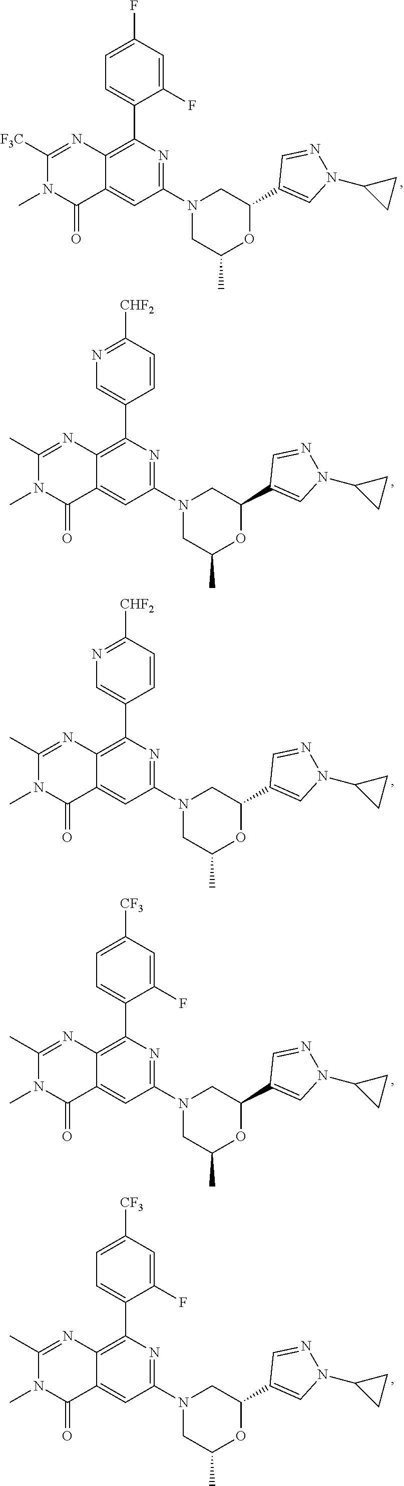 [SMILES: C[C@@H]1CN(c2cc3c(=O)n(C)c(C(F)(F)F)nc3c(-c3ccc(F)cc3F)n2)C[C@H](c2cnn(C3CC3)c2)O1 ; Cc1nc2c(-c3ccc(C(F)F)nc3)nc(N3C[C@@H](c4cnn(C5CC5)c4)O[C@@H](C)C3)cc2c(=O)n1C ; Cc1nc2c(-c3ccc(C(F)F)nc3)nc(N3C[C@@H](C)O[C@@H](c4cnn(C5CC5)c4)C3)cc2c(=O)n1C ; Cc1nc2c(-c3ccc(C(F)(F)F)cc3F)nc(N3C[C@@H](c4cnn(C5CC5)c4)O[C@@H](C)C3)cc2c(=O)n1C ; Cc1nc2c(-c3ccc(C(F)(F)F)cc3F)nc(N3C[C@@H](C)O[C@@H](c4cnn(C5CC5)c4)C3)cc2c(=O)n1C]

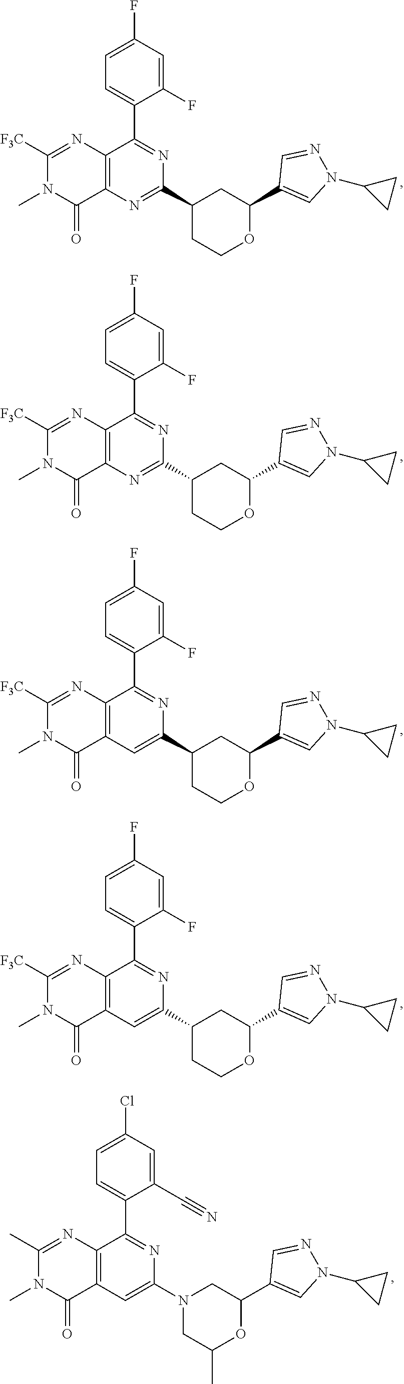 [SMILES: Cn1c(C(F)(F)F)nc2c(-c3ccc(F)cc3F)nc([C@@H]3CCO[C@H](c4cnn(C5CC5)c4)C3)nc2c1=O ; Cn1c(C(F)(F)F)nc2c(-c3ccc(F)cc3F)nc([C@H]3CCO[C@@H](c4cnn(C5CC5)c4)C3)nc2c1=O ; Cn1c(C(F)(F)F)nc2c(-c3ccc(F)cc3F)nc([C@@H]3CCO[C@H](c4cnn(C5CC5)c4)C3)cc2c1=O ; Cn1c(C(F)(F)F)nc2c(-c3ccc(F)cc3F)nc([C@H]3CCO[C@@H](c4cnn(C5CC5)c4)C3)cc2c1=O ; Cc1nc2c(-c3ccc(Cl)cc3C#N)nc(N3CC(C)OC(c4cnn(C5CC5)c4)C3)cc2c(=O)n1C]

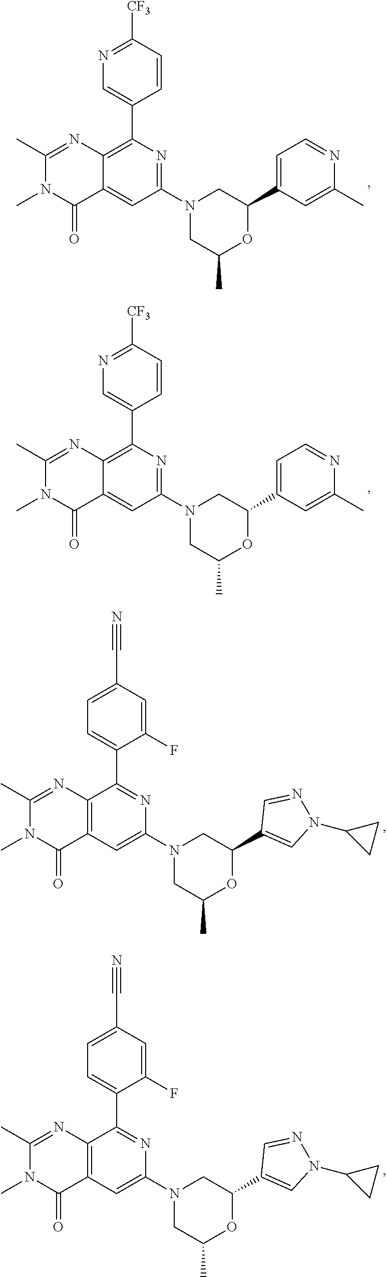

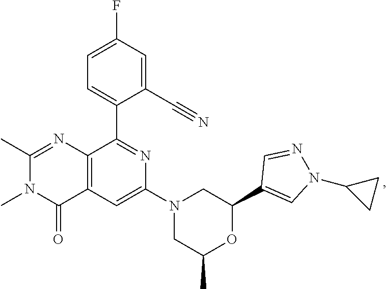 [SMILES: Cc1nc2c(-c3ccc(F)cc3C#N)nc(N3C[C@@H](c4cnn(C5CC5)c4)O[C@@H](C)C3)cc2c(=O)n1C]

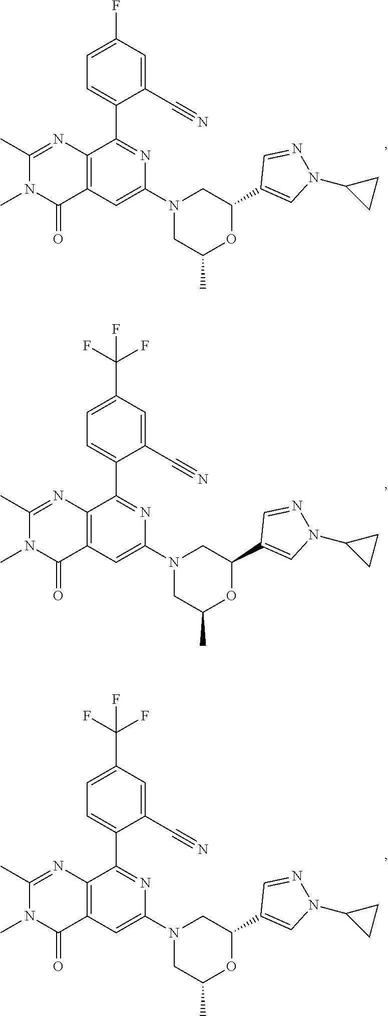 [SMILES: Cc1nc2c(-c3ccc(F)cc3C#N)nc(N3C[C@@H](C)O[C@@H](c4cnn(C5CC5)c4)C3)cc2c(=O)n1C ; Cc1nc2c(-c3ccc(C(F)(F)F)cc3C#N)nc(N3C[C@@H](c4cnn(C5CC5)c4)O[C@@H](C)C3)cc2c(=O)n1C ; Cc1nc2c(-c3ccc(C(F)(F)F)cc3C#N)nc(N3C[C@@H](C)O[C@@H](c4cnn(C5CC5)c4)C3)cc2c(=O)n1C]

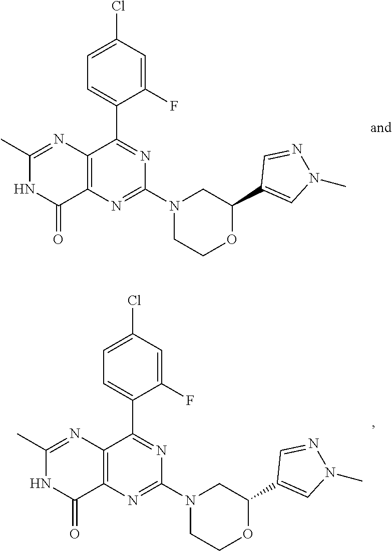

or a pharmaceutically acceptable salt thereof.

In one embodiment, the compound is 8-(4-chloro-2-fluorophenyl)-3-methyl-6-[(2R)-2-(1-methyl-1H-pyrazol-4-yl)morpholin-4-yl]-3H,4H-pyrimido[5,4-d][1,3]diazin-4-one or a pharmaceutically acceptable salt thereof. In one embodiment, the compound is 8-(4-chloro-2-fluorophenyl)-3-methyl-6-[(2S)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-6-[(2R)-2-(1-cyclopropyl-1H-pyrazol-4-yl)morpholin-4-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-6-[(2S)-2-(1-cyclopropyl-1H-pyrazol-4-yl)morpholin-4-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R)-2-(2-methylpyridin-4-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S)-2-(2-methylpyridin-4-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R)-2-(2-methylpyrimidin-5-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S)-2-(2-methylpyrimidin-5-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R)-2-(6-methylpyridazin-4-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S)-2-(6-methylpyridazin-4-yl)morpholin-4-yl]-3H,4H-pyrimido[5,4-d][1,3]diazin-4-one, or a pharmaceutically acceptable salt thereof. In one embodiment, the compound is 8-(4-chloro-2-fluorophenyl)-6-[(3R)-4,4-difluoro-3-(1-methyl-1H-pyrazol-4-yl)piperidin-1-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-6-[(3S)-4,4-difluoro-3-(1-methyl-1H-pyrazol-4-yl)piperidin-1-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S,4R)-2-(1-methyl-1H-pyrazol-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R,4S)-2-(1-methyl-1H-pyrazol-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R,4R)-2-(1-methyl-1H-pyrazol-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S,4S)-2-(1-methyl-1H-pyrazol-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-6-[(2S,4R)-2-(1-cyclopropyl-1H-pyrazol-4-yl)oxan-4-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-6-[(2R,4S)-2-(1-cyclopropyl-1H-pyrazol-4-yl)oxan-4-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-6-[(2R,4R)-2-(1-cyclopropyl-1H-pyrazol-4-yl)oxan-4-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-6-[(2S,4S)-2-(1-cyclopropyl-1H-pyrazol-4-yl)oxan-4-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S,4R)-2-(2-methylpyridin-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R,4S)-2-(2-methylpyridin-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R,4R)-2-(2-methylpyridin-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S,4S)-2-(2-methylpyridin-4-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S,4R)-2-(2-methylpyrimidin-5-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R,4S)-2-(2-methylpyrimidin-5-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2R,4R)-2-(2-methylpyrimidin-5-yl)oxan-4-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(2S,4S)-2-(2-methylpyrimidin-5-yl)oxan-4-yl]-3H,4H-pyrimido[5,4-d][1,3]diazin-4-one, or a pharmaceutically acceptable salt thereof. In one embodiment, the compound is 8-{6,6-difluorospiro[3.3]heptan-2-yl}-2,3-dimethyl-6-[(2R)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-{6,6-difluorospiro[3.3]heptan-2-yl}-2,3-dimethyl-6-[(2S)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-[(1r,3r)-3-(trifluoromethyl)cyclobutyl]-3H,4H-pyrimido[5,4-d][1,3]diazin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-[(1r,3r)-3-(trifluoromethyl)cyclobutyl]-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-[3-(trifluoromethyl)bicyclo[1.1.1]pentan-1-yl]-3H,4H-pyrimido[5,4-d][1,3]diazin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-[3-(trifluoromethyl)bicyclo[1.1.1]pentan-1-yl]-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-[6-(trifluoromethyl)pyridin-3-yl]-3H,4H-pyrimido[5,4-d][1,3]diazin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-[6-(trifluoromethyl)pyridin-3-yl]-3H,4H-pyrimido[5,4-d][1,3]diazin-4-one, or a pharmaceutically acceptable salt thereof. In one embodiment, the compound is 8-(4,4-difluorocyclohexyl)-2,3-dimethyl-6-[(2R)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-(4,4-difluorocyclohexyl)-2,3-dimethyl-6-[(2S)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-(2,4-difluorophenyl)-2,3-dimethyl-6-[(2R)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-(2,4-difluorophenyl)-2,3-dimethyl-6-[(2S)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-(2,3,4-trifluorophenyl)-3H,4H-pyrimido[5,4-d][1,3]diazin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-(2,3,4-trifluorophenyl)-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-(2,4,5-trifluorophenyl)-3H,4H-pyrimido[5,4-d][1,3]diazin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-(2,4,5-trifluorophenyl)-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-(3,4,5-trifluorophenyl)-3H,4H-pyrimido[5,4-d][1,3]diazin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-(3,4,5-trifluorophenyl)-3H,4H-pyrimido[5,4-d][1,3]diazin-4-one, or a pharmaceutically acceptable salt thereof. In one embodiment, the compound is 8-(3,4-difluorophenyl)-2,3-dimethyl-6-[(2R)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-(3,4-difluorophenyl)-2,3-dimethyl-6-[(2S)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-[(1r,4r)-4-methylcyclohexyl]-3H,4H-pyrimido[5,4-d][1,3]diazin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-[(1r,4r)-4-methylcyclohexyl]-3H,4H-pyrimido[5,4-d][1,3]diazin-4-one, or a pharmaceutically acceptable salt thereof. In one embodiment, the compound is 8-[2-fluoro-4-(trifluoromethyl)phenyl]-2,3-dimethyl-6-[(2R)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 8-[2-fluoro-4-(trifluoromethyl)phenyl]-2,3-dimethyl-6-[(S)-2-(1-methyl-1H-pyrazol-4-yl)morpholin-4-yl]-3H,4H-pyrimido[5,4-d][1,3]diazin-4-one, or a pharmaceutically acceptable salt thereof. In one embodiment, the compound is 2,3-dimethyl-6-[(2R)-2-(1-methyl-1H-pyrazol-4-yl)morpholin-4-yl]-8-(oxan-4-yl)-3H,4H,4aH,8aH-[1,3]diazino[5,4-d]pyrimidin-4-one, or a pharmaceutically acceptable salt thereof. In one embodiment, the compound is 2,3-dimethyl-6-[(2S)-2-(1-methyl-1H-pyrazol-4-yl)morpholin-4-yl]-8-(oxan-4-yl)-3H,4H,4aH,8aH-[1,3]diazino[5,4-d]pyrimidin-4-one, or a pharmaceutically acceptable salt thereof. In one embodiment, the compound is 8-(4,4-difluoropiperidin-1-yl)-2,3-dimethyl-6-[(2R)-2-(1-methyl-1H-pyrazol-4-yl)morpholin-4-yl]-3H,4H,4aH,8aH-[1,3]diazino[5,4-d]pyrimidin-4-one, or a pharmaceutically acceptable salt thereof. In one embodiment, the compound is 8-(4,4-difluoropiperidin-1-yl)-2,3-dimethyl-6-[(2S)-2-(1-methyl-1H-pyrazol-4-yl)morpholin-4-yl]-3H,4H,4aH,8aH-[1,3]diazino[5,4-d]pyrimidin-4-one, or a pharmaceutically acceptable salt thereof. In one embodiment, the compound is 8-(4-chloro-2-fluorophenyl)-2,3-dimethyl-6-{3-[(1-methyl-1H-pyrazol-4-yl)oxy]azetidin-1-yl}-3H,4H,4aH,8aH-[1,3]diazino[5,4-d]pyrimidin-4-one, or a pharmaceutically acceptable salt thereof. In one embodiment, the compound is 8-(4-chloro-2-fluorophenyl)-2,3-dimethyl-6-[(3R)-4-methyl-3-(1-methyl-1H-pyrazol-4-yl)piperazin-1-yl]-3H,4H-pyrimido[5,4-d][1,3]diazin-4-one, or a pharmaceutically acceptable salt thereof. In one embodiment, the compound is 8-(4-chloro-2-fluorophenyl)-2,3-dimethyl-6-[(3S)-4-methyl-3-(1-methyl-1H-pyrazol-4-yl)piperazin-1-yl]-3H,4H-pyrimido[5,4-d][1,3]diazin-4-one, or a pharmaceutically acceptable salt thereof. In one embodiment, the compound is 8-(4-chloro-2-fluorophenyl)-6-[(3R)-4-(2-fluoroethyl)-3-(1-methyl-1H-pyrazol-4-yl)piperazin-1-yl]-2,3-dimethyl-3H,4H-pyrimido[5,4-d][1,3]diazin-4-one, or a pharmaceutically acceptable salt thereof. In one embodiment, the compound is 8-(4-chloro-2-fluorophenyl)-6-[(3S)-4-(2-fluoroethyl)-3-(1-methyl-1H-pyrazol-4-yl)piperazin-1-yl]-2,3-dimethyl-3H,4H-pyrimido[5,4-d][1,3]diazin-4-one, or a pharmaceutically acceptable salt thereof. In one embodiment, the compound is 8-(4,4-difluorocyclohexyl)-2,3-dimethyl-6-(2-(1-methyl-1H-pyrazol-4-yl)morpholino)pyrimido[5,4-d]pyrimidin-4 (3H)-one or a pharmaceutically acceptable salt thereof. In one embodiment, the compound is 2,3-dimethyl-6-(2-(1-methyl-1H-pyrazol-4-yl)morpholino)-8-((1s,4s)-4-methylcyclohexyl)pyrimido[5,4-d]pyrimidin-4 (3H)-one or a pharmaceutically acceptable salt thereof. In one embodiment, the compound is 2,3-dimethyl-6-(2-(1-methyl-1H-pyrazol-4-yl)morpholino)-8-((1r,4r)-4-methylcyclohexyl)pyrimido[5,4-d]pyrimidin-4 (3H)-one or a pharmaceutically acceptable salt thereof. In one embodiment, the compound is 8-(4-chloro-2-fluorophenyl)-6-(4,4-difluoro-3-(1-methyl-1H-pyrazol-4-yl)piperidin-1-yl)-2,3-dimethylpyrimido[5,4-d]pyrimidin-4 (3H)-one or a pharmaceutically acceptable salt thereof. In one embodiment, the compound is 8-(4-chloro-2-fluorophenyl)-6-((2R,4S)-2-(1-cyclopropyl-1H-pyrazol-4-yl)tetrahydro-2H-pyran-4-yl)-2,3-dimethylpyrimido[5,4-d]pyrimidin-4 (3H)-one or a pharmaceutically acceptable salt thereof. In one embodiment, the compound is 8-(4-chloro-2-fluorophenyl)-2,3-dimethyl-6-(3-(((1-methyl-1H-pyrazol-4-yl)oxy)methyl)azetidin-1-yl)pyrimido[5,4-d]pyrimidin-4 (3H)-one or a pharmaceutically acceptable salt thereof. In one embodiment, the compound is 8-(4-chloro-2-fluorophenyl)-2,3-dimethyl-6-(3-((1-methyl-1H-pyrazol-4-yl)oxy)azetidin-1-yl)pyrimido[5,4-d]pyrimidin-4 (3H)-one or a pharmaceutically acceptable salt thereof. In one embodiment, the compound is 8-(4-chloro-2-fluorophenyl)-2,3-dimethyl-6-(2-(1-methyl-1H-pyrazol-4-yl)tetrahydro-2H-pyran-4-yl)pyrimido[5,4-d]pyrimidin-4 (3H)-one or a pharmaceutically acceptable salt thereof. In one embodiment, the compound is 8-(4-chloro-2-fluorophenyl)-2,3-dimethyl-6-(3-((1-methyl-1H-pyrazol-4-yl)methoxy)azetidin-1-yl)pyrimido[5,4-d]pyrimidin-4 (3H)-one or a pharmaceutically acceptable salt thereof. In one embodiment, the compound is (R)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is (S)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is (R)-6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is (S)-6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is (R)-8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one, or a pharmaceutically acceptable salt thereof. In one embodiment, the compound is (S)-8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one, or a pharmaceutically acceptable salt thereof. In one embodiment, the compound is (R)-2,3-dimethyl-6-(2-(2-methylpyridin-4-yl)morpholino)-8-(6-(trifluoromethyl)pyridin-3-yl)pyrimido[5,4-d]pyrimidin-4 (3H)-one, or a pharmaceutically acceptable salt thereof. In one embodiment, the compound is (S)-2,3-dimethyl-6-(2-(2-methylpyridin-4-yl)morpholino)-8-(6-(trifluoromethyl)pyridin-3-yl)pyrimido[5,4-d]pyrimidin-4 (3H)-one, or a pharmaceutically acceptable salt thereof. In one embodiment, the compound is (R)-8-(4-chloro-2-fluorophenyl)-2,3-dimethyl-6-(2-(2-methylpyridin-4-yl)morpholino)pyrimido[5,4-d]pyrimidin-4 (3H)-one, or a pharmaceutically acceptable salt thereof. In one embodiment, the compound is (S)-8-(4-chloro-2-fluorophenyl)-2,3-dimethyl-6-(2-(2-methylpyridin-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-6-((2S,4R)-2-(1-cyclopropyl-1H-pyrazol-4-yl)tetrahydro-2H-pyran-4-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-((2R,4S)-2-(1-cyclopropyl-1H-pyrazol-4-yl)tetrahydro-2H-pyran-4-yl)-2,3-dimethylpyrimido[5,4-d]pyrimidin-4 (3H)-one, or a pharmaceutically acceptable salt thereof. In one embodiment, the compound is (R)-8-(2-fluoro-4-(trifluoromethyl)phenyl)-2,3-dimethyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is (S)-8-(2-fluoro-4-(trifluoromethyl)phenyl)-2,3-dimethyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is (R)-8-(4-chloro-2-fluorophenyl)-6-(2-(1-cyclopropyl-1H-pyrazol-4-yl)morpholino)-2,3-dimethylpyrimido[5,4-d]pyrimidin-4 (3H)-one, or a pharmaceutically acceptable salt thereof. In one embodiment, the compound is (S)-8-(4-chloro-2-fluorophenyl)-6-(2-(1-cyclopropyl-1H-pyrazol-4-yl)morpholino)-2,3-dimethylpyrimido[5,4-d]pyrimidin-4 (3H)-one, or a pharmaceutically acceptable salt thereof. In one embodiment, the compound is (R)-2,3-dimethyl-6-(2-(1-methyl-1H-pyrazol-4-yl)morpholino)-8-(2,4,5-trifluorophenyl)pyrimido[5,4-d]pyrimidin-4 (3H)-one, or a pharmaceutically acceptable salt thereof. In one embodiment, the compound is (S)-2,3-dimethyl-6-(2-(1-methyl-1H-pyrazol-4-yl)morpholino)-8-(2,4,5-trifluorophenyl)pyrimido[5,4-d]pyrimidin-4 (3H)-one, or a pharmaceutically acceptable salt thereof. In one embodiment, the compound is (R)-2,3-dimethyl-6-(2-(1-methyl-1H-pyrazol-4-yl)morpholino)-8-(6-(trifluoromethyl)pyridin-3-yl)pyrimido[5,4-d]pyrimidin-4 (3H)-one, or a pharmaceutically acceptable salt thereof. In one embodiment, the compound is (S)-2,3-dimethyl-6-(2-(1-methyl-1H-pyrazol-4-yl)morpholino)-8-(6-(trifluoromethyl)pyridin-3-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(4-methyl-3-(1-methyl-1H-pyrazol-4-yl)piperazin-1-yl)pyrimido[5,4-d]pyrimidin-4 (3H)-one, or a pharmaceutically acceptable salt thereof. In one embodiment, the compound is 8-(4-chloro-2-fluorophenyl)-6-(4-ethyl-3-(1-methyl-1H-pyrazol-4-yl)piperazin-1-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-(4-(2-fluoroethyl)-3-(1-methyl-1H-pyrazol-4-yl)piperazin-1-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-(4-(2,2-difluoroethyl)-3-(1-methyl-1H-pyrazol-4-yl)piperazin-1-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(3-(1-methyl-1H-pyrazol-4-yl)-4-(2,2,2-trifluoroethyl)piperazin-1-yl)pyrimido[5,4-d]pyrimidin-4 (3H)-one, or a pharmaceutically acceptable salt thereof. In one embodiment, the compound is 8-(4-chloro-2-fluorophenyl)-6-(4-(2-methoxyethyl)-3-(1-methyl-1H-pyrazol-4-yl)piperazin-1-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(2-methylpyrimidin-5-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-pyrazol-3-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-((2R,4S)-2-(2-methylpyrimidin-5-yl)tetrahydro-2H-pyran-4-yl)pyrimido[5,4-d]pyrimidin-4 (3H)-one, or a pharmaceutically acceptable salt thereof. In one embodiment, the compound is 2,3-dimethyl-6-((2S,4R)-2-(1-methyl-1H-pyrazol-4-yl)tetrahydro-2H-pyran-4-yl)-8-(2,4,5-trifluorophenyl)pyrimido[5,4-d]pyrimidin-4 (3H)-one, or a pharmaceutically acceptable salt thereof. In one embodiment, the compound is 2,3-dimethyl-6-((2R,4S)-2-(1-methyl-1H-pyrazol-4-yl)tetrahydro-2H-pyran-4-yl)-8-(2,4,5-trifluorophenyl)pyrimido[5,4-d]pyrimidin-4 (3H)-one, or a pharmaceutically acceptable salt thereof. In one embodiment, the compound is 6-(2-(1H-pyrazol-4-yl)morpholino)-8-(4-chloro-2-fluorophenyl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-(oxetan-3-yl)-1H-pyrazol-4-yl)morpholino)pyrimido[5,4-d]pyrimidin-4 (3H)-one, or a pharmaceutically acceptable salt thereof. In one embodiment, the compound is 6-(2-(1-(azetidin-3-yl)-1H-pyrazol-4-yl)morpholino)-8-(4-chloro-2-fluorophenyl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-(1-methylazetidin-3-yl)-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(5-methyl-1,3,4-oxadiazol-2-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-pyrazol-5-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(4-methylthiazol-2-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(3-methyl-1H-1,2,4-triazol-5-yl)morpholino)pyrimido[5,4-d]pyrimidin-4 (3H)-one, or a pharmaceutically acceptable salt thereof. In one embodiment, the compound is 6-(2-(1H-pyrazol-3-yl)morpholino)-8-(4-chloro-2-fluorophenyl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(thiazol-2-yl)morpholino)pyrimido[5,4-d]pyrimidin-4 (3H)-one, or a pharmaceutically acceptable salt thereof. In one embodiment, the compound is 4-(4-(4-chloro-2-fluorophenyl)-6,7-dimethyl-8-oxo-7,8-dihydropyrimido[5,4-d]pyrimidin-2-yl)-N-methylmorpholine-2-carboxamide, or a pharmaceutically acceptable salt thereof. In one embodiment, the compound is 8-(4-chloro-2-fluorophenyl)-2,3-dimethyl-6-(2-(5-methyl-1,2,4-oxadiazol-3-yl)morpholino)pyrimido[5,4-d]pyrimidin-4 (3H)-one, or a pharmaceutically acceptable salt thereof. In one embodiment, the compound is 4-(4-(4-chloro-2-fluorophenyl)-6,7-dimethyl-8-oxo-7,8-dihydropyrimido[5,4-d]pyrimidin-2-yl)-N,N-dimethylmorpholine-2-carboxamide, or a pharmaceutically acceptable salt thereof. In one embodiment, the compound is 8-(4-chloro-2-fluorophenyl)-6-(2-(methoxymethyl)morpholino)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-(2-((dimethylamino)methyl)morpholino)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(6-oxa-9-azaspiro[4.5]decan-9-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-methyl-2-(1-methyl-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(9-methyl-1-oxa-4,9-diazaspiro[5.5]undecan-4-yl)pyrimido[5,4-d]pyrimidin-4 (3H)-one, or a pharmaceutically acceptable salt thereof. In one embodiment, the compound is 8-(4-chloro-2-fluorophenyl)-6-(2,2-dimethylmorpholino)-2,3-dimethylpyrimido[5,4-d]pyrimidin-4 (3H)-one, or a pharmaceutically acceptable salt thereof. In one embodiment, the compound is 6-(2-(1,2,4-oxadiazol-3-yl)morpholino)-8-(4-chloro-2-fluorophenyl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-3-(4-methoxybenzyl)-2-methyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2-methyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-3-(2-methoxyethyl)-2-methyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-[3-(1-methyl-1H-pyrazol-4-yl)pyrrolidin-1-yl]-3H,4H-pyrimido[5,4-d][1,3]diazin-4-one, or a pharmaceutically acceptable salt thereof. In one embodiment, the compound is (R)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one, or a pharmaceutically acceptable salt thereof. In one embodiment, the compound is 8-(2-fluoro-4-(trifluoromethyl)phenyl)-2,3-dimethyl-6-((2S,4R)-2-(1-methyl-1H-pyrazol-4-yl)tetrahydro-2H-pyran-4-yl)pyrimido[5,4-d]pyrimidin-4 (3H)-one, or a pharmaceutically acceptable salt thereof. In one embodiment, the compound is 8-(2-fluoro-4-(trifluoromethyl)phenyl)-2,3-dimethyl-6-((2R,4S)-2-(1-methyl-1H-pyrazol-4-yl)tetrahydro-2H-pyran-4-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-((2S,4R)-2-(1-methyl-1H-pyrazol-4-yl)tetrahydro-2H-pyran-4-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-((2R,4S)-2-(1-methyl-1H-pyrazol-4-yl)tetrahydro-2H-pyran-4-yl)pyrimido[5,4-d]pyrimidin-4 (3H)-one, or a pharmaceutically acceptable salt thereof. In one embodiment, the compound is 8-(4-chloro-2-fluorophenyl)-6-(2-(1-(2-methoxyethyl)-1H-pyrazol-4-yl)morpholino)-2,3-dimethylpyrimido[5,4-d]pyrimidin-4 (3H)-one, or a pharmaceutically acceptable salt thereof. In one embodiment, the compound is 8-(2-chloro-4-fluorophenyl)-2,3-dimethyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is 2,3-dimethyl-6-((R)-2-(1-methyl-1H-pyrazol-4-yl)morpholino)-8-((1r,4R)-4-methylcyclohexyl)pyrimido[5,4-d]pyrimidin-4 (3H)-one, or a pharmaceutically acceptable salt thereof. In one embodiment, the compound is 2,3-dimethyl-6-((S)-2-(1-methyl-1H-pyrazol-4-yl)morpholino)-8-((1r,4S)-4-methylcyclohexyl)pyrimido[5,4-d]pyrimidin-4 (3H)-one, or a pharmaceutically acceptable salt thereof. In one embodiment, the compound is 8-(6,6-difluorospiro[3.3]heptan-2-yl)-2,3-dimethyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-1,2,3-triazol-4-yl)morpholino)pyrimido[5,4-d]pyrimidin-4 (3H)-one, or a pharmaceutically acceptable salt thereof. In one embodiment, the compound is (S)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one, or a pharmaceutically acceptable salt thereof. In one embodiment, the compound is (S)-8-(4-chloro-2-fluorophenyl)-2,3-dimethyl-6-(3-((1-methyl-1H-pyrazol-4-yl)oxy)piperidin-1-yl)pyrimido[5,4-d]pyrimidin-4 (3H)-one, or a pharmaceutically acceptable salt thereof. In one embodiment, the compound is (R)-8-(4-chloro-2-fluorophenyl)-2,3-dimethyl-6-(3-((1-methyl-1H-pyrazol-4-yl)oxy)piperidin-1-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(4-((1-methyl-1H-pyrazol-4-yl)oxy)piperidin-1-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(3-morpholinopiperidin-1-yl)pyrimido[5,4-d]pyrimidin-4 (3H)-one, or a pharmaceutically acceptable salt thereof. In one embodiment, the compound is 8-(4-chloro-2-fluorophenyl)-6-(3-(3-methoxyazetidin-1-yl)piperidin-1-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-(3-(3,3-difluoroazetidin-1-yl)piperidin-1-yl)-2,3-dimethylpyrimido[5,4-d]pyrimidin-4 (3H)-one, or a pharmaceutically acceptable salt thereof. In one embodiment, the compound is 6-(2-(1H-imidazol-2-yl)morpholino)-8-(4-chloro-2-fluorophenyl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-imidazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(5,6,8,9-tetrahydro-7H-pyrimido[4,5-d]azepin-7-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(4-((3-methylpyridin-4-yl)oxy)piperidin-1-yl)pyrimido[5,4-d]pyrimidin-4 (3H)-one, or a pharmaceutically acceptable salt thereof. In one embodiment, the compound is 8-(4-chloro-2-fluorophenyl)-6-(2,3-dihydrospiro[indene-1,2′-morpholin]-4′-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(5,6,7,9-tetrahydro-8H-pyrido[3,4-c]azepin-8-yl)pyrimido[5,4-d]pyrimidin-4 (3H)-one, or a pharmaceutically acceptable salt thereof. In one embodiment, the compound is 2,3-dimethyl-6-(2-(1-methyl-1H-pyrazol-4-yl)morpholino)-8-(piperidin-4-yl)pyrimido[5,4-d]pyrimidin-4 (3H)-one, or a pharmaceutically acceptable salt thereof. In one embodiment, the compound is 2,3-dimethyl-6-((2S,4R)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one, or a pharmaceutically acceptable salt thereof. In one embodiment, the compound is 2,3-dimethyl-6-((2R,4S)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one, or a pharmaceutically acceptable salt thereof. In one embodiment, the compound is (R)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is (S)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(6-(1-methyl-1H-pyrazol-4-yl)-5-oxa-8-azaspiro(3,5)nonan-8-yl)pyrimido(5,4-d)pyrimidin-4 (3H)-one, or a pharmaceutically acceptable salt thereof. In one embodiment, the compound is 8-(4-chloro-2-fluorophenyl)-2,3-dimethyl-6-(2-(2-methyl-2H-1,2,3-triazol-4-yl)morpholino)pyrimido[5,4-d]pyrimidin-4 (3H)-one, or a pharmaceutically acceptable salt thereof. In one embodiment, the compound is (R)-6-(4,4-difluoro-3-(1-methyl-1H-pyrazol-4-yl)piperidin-1-yl)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is (S)-6-(4,4-difluoro-3-(1-methyl-1H-pyrazol-4-yl)piperidin-1-yl)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-((2R,4S)-2-(2-methylpyridin-4-yl)tetrahydro-2H-pyran-4-yl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-((2R,4R)-2-(2-methylpyridin-4-yl)tetrahydro-2H-pyran-4-yl)pyrimido[5,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-2,3-dimethyl-8-(2,4,5-trifluorophenyl)pyrimido[5,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-2,3-dimethyl-8-(2,4,5-trifluorophenyl)pyrimido[5,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is 2,3-dimethyl-6-(2-(1-methyl-1H-pyrazol-4-yl)morpholino)-8-(1-methyl-3-(trifluoromethyl)-1H-pyrazol-5-yl)pyrimido[5,4-d]pyrimidin-4 (3H)-one, or a pharmaceutically acceptable salt thereof. In one embodiment, the compound is (R)-6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one, or a pharmaceutically acceptable salt thereof. In one embodiment, the compound is (S)-6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one, or a pharmaceutically acceptable salt thereof. In one embodiment, the compound is (R)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one, or a pharmaceutically acceptable salt thereof. In one embodiment, the compound is (S)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one, or a pharmaceutically acceptable salt thereof. In one embodiment, the compound is (R)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is (S)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is (S)-8-(4-chloro-2-fluorophenyl)-3-methyl-6-(2-(1-methyl-1H-pyrazol-4-yl)morpholino)-2-(trifluoromethyl)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-pyrazol-4-yl)morpholino)pyrido[3,2-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-pyrazol-4-yl)morpholino)pyrido[3,2-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-pyrazol-4-yl)morpholino)quinazolin-4 (3H)-one, or a pharmaceutically acceptable salt thereof. In one embodiment, the compound is (R)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one, or a pharmaceutically acceptable salt thereof. In one embodiment, the compound is (S)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(5-(1-methyl-1H-pyrazol-4-yl)-4-oxa-7-azaspiro(2,5)octan-7-yl)pyrimido(5,4-d)pyrimidin-4 (3H)-one, or a pharmaceutically acceptable salt thereof. In one embodiment, the compound is (R)-8-(4-chloro-2-fluorophenyl)-2,3-dimethyl-6-(5-(1-methyl-1H-pyrazol-4-yl)-4-oxa-7-azaspiro[2.5]octan-7-yl)pyrimido[5,4-d]pyrimidin-4 (3H)-one, or a pharmaceutically acceptable salt thereof. In one embodiment, the compound is (S)-8-(4-chloro-2-fluorophenyl)-2,3-dimethyl-6-(5-(1-methyl-1H-pyrazol-4-yl)-4-oxa-7-azaspiro[2.5]octan-7-yl)pyrimido[5,4-d]pyrimidin-4 (3H)-one, or a pharmaceutically acceptable salt thereof. In one embodiment, the compound is (R)-8-(4-chloro-2-fluorophenyl)-2,3-dimethyl-6-(6-(1-methyl-1H-pyrazol-4-yl)-5-oxa-8-azaspiro[3.5]nonan-8-yl)pyrimido[5,4-d]pyrimidin-4 (3H)-one, or a pharmaceutically acceptable salt thereof. In one embodiment, the compound is (S)-8-(4-chloro-2-fluorophenyl)-2,3-dimethyl-6-(6-(1-methyl-1H-pyrazol-4-yl)-5-oxa-8-azaspiro[3.5]nonan-8-yl)pyrimido[5,4-d]pyrimidin-4 (3H)-one, or a pharmaceutically acceptable salt thereof. In one embodiment, the compound is (R)-8-(4-chloro-2-fluorophenyl)-6-(6-(1-cyclopropyl-1H-pyrazol-4-yl)-2,2-dimethylmorpholino)-2,3-dimethylpyrimido[5,4-d]pyrimidin-4 (3H)-one, or a pharmaceutically acceptable salt thereof. In one embodiment, the compound is (S)-8-(4-chloro-2-fluorophenyl)-6-(6-(1-cyclopropyl-1H-pyrazol-4-yl)-2,2-dimethylmorpholino)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-((4S,6S)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-((4R,6R)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one, or a pharmaceutically acceptable salt thereof. In one embodiment, the compound is (R)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one, or a pharmaceutically acceptable salt thereof. In one embodiment, the compound is (S) (R)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one, or a pharmaceutically acceptable salt thereof. In one embodiment, the compound is (S)-2,3-dimethyl-6-(2-(1-methyl-1H-pyrazol-4-yl)morpholino)-8-(3-(trifluoromethyl)bicyclo[1.1.1]pentan-1-yl)pyrimido[5,4-d]pyrimidin-4 (3H)-one, or a pharmaceutically acceptable salt thereof. In one embodiment, the compound is 4-(4-chloro-2-fluorophenyl)-6,7-dimethyl-2-(2-(1-methyl-1H-pyrazol-4-yl)morpholino)pyrido[3,4-d]pyrimidin-8 (7H)-one, or a pharmaceutically acceptable salt thereof. In one embodiment, the compound is 8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-(2-(1-cyclopropyl-1H-pyrazol-4-yl)-3-oxabicyclo[4.1.0]heptan-6-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-((1S,2S,6R)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-((1R,2R,6S)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one, or a pharmaceutically acceptable salt thereof. In one embodiment, the compound is (R)-8-(4-chloro-2-fluorophenyl)-6-(2,2-dimethyl-6-(1-methyl-1H-pyrazol-4-yl)morpholino)-2,3-dimethylquinazolin-4 (3H)-one, or a pharmaceutically acceptable salt thereof. In one embodiment, the compound is (S)-8-(4-chloro-2-fluorophenyl)-6-(2,2-dimethyl-6-(1-methyl-1H-pyrazol-4-yl)morpholino)-2,3-dimethylquinazolin-4 (3H)-one, or a pharmaceutically acceptable salt thereof. In one embodiment, the compound is 6-(2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one, or a pharmaceutically acceptable salt thereof. In one embodiment, the compound is 8-(4-chloro-2-fluorophenyl)-6-((2S,4R)-2-(1-cyclopropyl-1H-pyrazol-4-yl)tetrahydro-2H-pyran-4-yl)-2,3-dimethylquinazolin-4 (3H)-one, or a pharmaceutically acceptable salt thereof. In one embodiment, the compound is 8-(4-chloro-2-fluorophenyl)-6-((2R,4S)-2-(1-cyclopropyl-1H-pyrazol-4-yl)tetrahydro-2H-pyran-4-yl)-2,3-dimethylquinazolin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one, or a pharmaceutically acceptable salt thereof. In one embodiment, the compound is 8-(2,4-difluorophenyl)-6-(2,2-dimethyl-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is (R)-8-(2,4-difluorophenyl)-6-(2,2-dimethyl-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is (S)-8-(2,4-difluorophenyl)-6-(2,2-dimethyl-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is 8-(2,4-difluorophenyl)-2,3-dimethyl-6-(2-(2-methylpyridin-4-yl)morpholino)pyrimido[5,4-d]pyrimidin-4 (3H)-one, or a pharmaceutically acceptable salt thereof. In one embodiment, the compound is (R)-8-(2,4-difluorophenyl)-2,3-dimethyl-6-(2-(2-methylpyridin-4-yl)morpholino)pyrimido[5,4-d]pyrimidin-4 (3H)-one, or a pharmaceutically acceptable salt thereof. In one embodiment, the compound is (S)-8-(2,4-difluorophenyl)-2,3-dimethyl-6-(2-(2-methylpyridin-4-yl)morpholino)pyrimido[5,4-d]pyrimidin-4 (3H)-one, or a pharmaceutically acceptable salt thereof. In one embodiment, the compound is 2,3-dimethyl-6-(2-(2-methylpyridin-4-yl)morpholino)-8-(2,4,5-trifluorophenyl)pyrimido[5,4-d]pyrimidin-4 (3H)-one, or a pharmaceutically acceptable salt thereof. In one embodiment, the compound is (R)-2,3-dimethyl-6-(2-(2-methylpyridin-4-yl)morpholino)-8-(2,4,5-trifluorophenyl)pyrimido[5,4-d]pyrimidin-4 (3H)-one, or a pharmaceutically acceptable salt thereof. In one embodiment, the compound is (S)-2,3-dimethyl-6-(2-(2-methylpyridin-4-yl)morpholino)-8-(2,4,5-trifluorophenyl)pyrimido[5,4-d]pyrimidin-4 (3H)-one, or a pharmaceutically acceptable salt thereof. In one embodiment, the compound is 6-(2,2-dimethyl-6-(2-methylpyridin-4-yl)morpholino)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is (R)-6-(2,2-dimethyl-6-(2-methylpyridin-4-yl)morpholino)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is (S)-6-(2,2-dimethyl-6-(2-methylpyridin-4-yl)morpholino)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is (S)-8-(4-chloro-2-fluorophenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is (R)-8-(4-chloro-2-fluorophenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is 8-(2-fluoro-4-(trifluoromethyl)phenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is (S)-8-(2-fluoro-4-(trifluoromethyl)phenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is (R)-8-(2-fluoro-4-(trifluoromethyl)phenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is 2,3-dimethyl-6-(2-(1-methyl-1H-pyrazol-4-yl)morpholino)-8-(6-(trifluoromethyl)pyridin-3-yl)pyrido[3,4-d]pyrimidin-4 (3H)-one, or a pharmaceutically acceptable salt thereof. In one embodiment, the compound is (S)-2,3-dimethyl-6-(2-(1-methyl-1H-pyrazol-4-yl)morpholino)-8-(6-(trifluoromethyl)pyridin-3-yl)pyrido[3,4-d]pyrimidin-4 (3H)-one, or a pharmaceutically acceptable salt thereof. In one embodiment, the compound is (R)-2,3-dimethyl-6-(2-(1-methyl-1H-pyrazol-4-yl)morpholino)-8-(6-(trifluoromethyl)pyridin-3-yl)pyrido[3,4-d]pyrimidin-4 (3H)-one, or a pharmaceutically acceptable salt thereof. In one embodiment, the compound is (R)-8-(4-chloro-2-fluorophenyl)-2,3-dimethyl-6-(5-(2-methylpyridin-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (S)-8-(4-chloro-2-fluorophenyl)-2,3-dimethyl-6-(5-(2-methylpyridin-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (S)-8-(2,4-difluorophenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is (R)-8-(2,4-difluorophenyl)-2,3-dimethyl-6-(2-(1-methyl-1H-pyrazol-4-yl)morpholino)pyrido[3,4-d]pyrimidin-4 (3H)-one, or a pharmaceutically acceptable salt thereof. In one embodiment, the compound is (S)-2,3-dimethyl-6-(2-(1-methyl-1H-pyrazol-4-yl)morpholino)-8-(2,4,5-trifluorophenyl)pyrido[3,4-d]pyrimidin-4 (3H)-one, or a pharmaceutically acceptable salt thereof. In one embodiment, the compound is (R)-2,3-dimethyl-6-(2-(1-methyl-1H-pyrazol-4-yl)morpholino)-8-(2,4,5-trifluorophenyl)pyrido[3,4-d]pyrimidin-4 (3H)-one, or a pharmaceutically acceptable salt thereof. In one embodiment, the compound is (R)-8-(4-chloro-2-fluorophenyl)-6-(2-(1-cyclopropyl-1H-pyrazol-4-yl)morpholino)-2,3-dimethylpyrido[3,4-d]pyrimidin-4 (3H)-one, or a pharmaceutically acceptable salt thereof. In one embodiment, the compound is (S)-8-(4-chloro-2-fluorophenyl)-6-(2-(1-cyclopropyl-1H-pyrazol-4-yl)morpholino)-2,3-dimethylpyrido[3,4-d]pyrimidin-4 (3H)-one, or a pharmaceutically acceptable salt thereof. In one embodiment, the compound is (R)-6-(2-(1-cyclopropyl-1H-pyrazol-4-yl)morpholino)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is (S)-6-(2-(1-cyclopropyl-1H-pyrazol-4-yl)morpholino)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is (R)-6-(2-(1-cyclopropyl-1H-pyrazol-4-yl)morpholino)-2,3-dimethyl-8-(2,4,5-trifluorophenyl)pyrido[3,4-d]pyrimidin-4 (3H)-one, or a pharmaceutically acceptable salt thereof. In one embodiment, the compound is (S)-6-(2-(1-cyclopropyl-1H-pyrazol-4-yl)morpholino)-2,3-dimethyl-8-(2,4,5-trifluorophenyl)pyrido[3,4-d]pyrimidin-4 (3H)-one, or a pharmaceutically acceptable salt thereof. In one embodiment, the compound is (R)-6-(2,2-dimethyl-6-(1-methyl-1H-pyrazol-4-yl)morpholino)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is (S)-6-(2,2-dimethyl-6-(1-methyl-1H-pyrazol-4-yl)morpholino)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is (R)-8-(4-chloro-2-fluorophenyl)-2,3-dimethyl-6-(2-(2-methylpyridin-4-yl)morpholino)pyrido[3,4-d]pyrimidin-4 (3H)-one, or a pharmaceutically acceptable salt thereof. In one embodiment, the compound is (S)-8-(4-chloro-2-fluorophenyl)-2,3-dimethyl-6-(2-(2-methylpyridin-4-yl)morpholino)pyrido[3,4-d]pyrimidin-4 (3H)-one, or a pharmaceutically acceptable salt thereof. In one embodiment, the compound is (R)-8-(2,4-difluorophenyl)-2,3-dimethyl-6-(2-(2-methylpyridin-4-yl)morpholino)pyrido[3,4-d]pyrimidin-4 (3H)-one, or a pharmaceutically acceptable salt thereof. In one embodiment, the compound is (S)-8-(2,4-difluorophenyl)-2,3-dimethyl-6-(2-(2-methylpyridin-4-yl)morpholino)pyrido[3,4-d]pyrimidin-4 (3H)-one, or a pharmaceutically acceptable salt thereof. In one embodiment, the compound is (R)-8-(2-fluoro-4-(trifluoromethyl)phenyl)-2,3-dimethyl-6-(2-(2-methylpyridin-4-yl)morpholino)pyrido[3,4-d]pyrimidin-4 (3H)-one, or a pharmaceutically acceptable salt thereof. In one embodiment, the compound is (S)-8-(2-fluoro-4-(trifluoromethyl)phenyl)-2,3-dimethyl-6-(2-(2-methylpyridin-4-yl)morpholino)pyrido[3,4-d]pyrimidin-4 (3H)-one, or a pharmaceutically acceptable salt thereof. In one embodiment, the compound is (R)-2,3-dimethyl-6-(2-(2-methylpyridin-4-yl)morpholino)-8-(6-(trifluoromethyl)pyridin-3-yl)pyrido[3,4-d]pyrimidin-4 (3H)-one, or a pharmaceutically acceptable salt thereof. In one embodiment, the compound is (S)-2,3-dimethyl-6-(2-(2-methylpyridin-4-yl)morpholino)-8-(6-(trifluoromethyl)pyridin-3-yl)pyrido[3,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-2,3-dimethyl-8-(2,4,5-trifluorophenyl)pyrido[3,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-2,3-dimethyl-8-(2,4,5-trifluorophenyl)pyrido[3,4-d]pyrimidin-4 (3H)-one, or a pharmaceutically acceptable salt thereof. In one embodiment, the compound is 8-(4-chloro-2-fluorophenyl)-6-((2S,4R)-2-(1-cyclopropyl-1H-pyrazol-4-yl)tetrahydro-2H-pyran-4-yl)-2,3-dimethylpyrido[3,4-d]pyrimidin-4 (3H)-one, or a pharmaceutically acceptable salt thereof. In one embodiment, the compound is 8-(4-chloro-2-fluorophenyl)-6-((2R,4S)-2-(1-cyclopropyl-1H-pyrazol-4-yl)tetrahydro-2H-pyran-4-yl)-2,3-dimethylpyrido[3,4-d]pyrimidin-4 (3H)-one, or a pharmaceutically acceptable salt thereof. In one embodiment, the compound is (R)-2,3-dimethyl-6-(2-(2-methylpyridin-4-yl)morpholino)-8-(2,4,5-trifluorophenyl)pyrido[3,4-d]pyrimidin-4 (3H)-one, or a pharmaceutically acceptable salt thereof. In one embodiment, the compound is (S)-2,3-dimethyl-6-(2-(2-methylpyridin-4-yl)morpholino)-8-(2,4,5-trifluorophenyl)pyrido[3,4-d]pyrimidin-4 (3H)-one, or a pharmaceutically acceptable salt thereof. In one embodiment, the compound is (R)8-(4-chloro-2-fluorophenyl)-6-(2,2-dimethyl-6-(1-methyl-1H-pyrazol-4-yl)morpholino)-2,3-dimethylpyrido[3,2-d]pyrimidin-4 (3H)-one, or a pharmaceutically acceptable salt thereof. In one embodiment, the compound is (S)8-(4-chloro-2-fluorophenyl)-6-(2,2-dimethyl-6-(1-methyl-1H-pyrazol-4-yl)morpholino)-2,3-dimethylpyrido[3,2-d]pyrimidin-4 (3H)-one, or a pharmaceutically acceptable salt thereof. In one embodiment, the compound is (R)-8-(2-fluoro-4-(trifluoromethyl)phenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one, or a pharmaceutically acceptable salt thereof. In one embodiment, the compound is (S)-8-(2-fluoro-4-(trifluoromethyl)phenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one, or a pharmaceutically acceptable salt thereof. In one embodiment, the compound is (R)-8-(4-chloro-2-fluorophenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (S)-8-(4-chloro-2-fluorophenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (R)-8-(2-fluoro-4-(trifluoromethyl)phenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (S)-8-(2-fluoro-4-(trifluoromethyl)phenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (R)-2,3-dimethyl-6-(5-(1-methyl-1H-pyrazol-4-yl)-4-oxa-7-azaspiro[2.5]octan-7-yl)-8-(2,4,5-trifluorophenyl)pyrido[3,4-d]pyrimidin-4 (3H)-one, or a pharmaceutically acceptable salt thereof. In one embodiment, the compound is (S)-2,3-dimethyl-6-(5-(1-methyl-1H-pyrazol-4-yl)-4-oxa-7-azaspiro[2.5]octan-7-yl)-8-(2,4,5-trifluorophenyl)pyrido[3,4-d]pyrimidin-4 (3H)-one, or a pharmaceutically acceptable salt thereof. In one embodiment, the compound is (S)-8-(4-chloro-2-fluorophenyl)-6-(2,2-dimethyl-6-(2-methyl-2H-1,2,3-triazol-4-yl)morpholino)-2,3-dimethylpyrido[3,4-d]pyrimidin-4 (3H)-one, or a pharmaceutically acceptable salt thereof. In one embodiment, the compound is (R)-8-(4-chloro-2-fluorophenyl)-6-(2,2-dimethyl-6-(2-methyl-2H-1,2,3-triazol-4-yl)morpholino)-2,3-dimethylpyrido[3,4-d]pyrimidin-4 (3H)-one, or a pharmaceutically acceptable salt thereof. In one embodiment, the compound is 8-(4-chloro-2-fluorophenyl)-2,3-dimethyl-6-((1R,2S)-2-(1-methyl-1H-pyrazol-4-yl)cyclopropyl)pyrido[3,4-d]pyrimidin-4 (3H)-one, or a pharmaceutically acceptable salt thereof. In one embodiment, the compound is 8-(4-chloro-2-fluorophenyl)-2,3-dimethyl-6-((1R,2R)-2-(1-methyl-1H-pyrazol-4-yl)cyclopropyl)pyrido[3,4-d]pyrimidin-4 (3H)-one, or a pharmaceutically acceptable salt thereof. In one embodiment, the compound is (R)-8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is (S)-8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one, or a pharmaceutically acceptable salt thereof. In one embodiment, the compound is (R)-6-(2,2-dimethyl-6-(1-methyl-1H-pyrazol-4-yl)morpholino)-2,3-dimethyl-8-(2,4,5-trifluorophenyl)pyrido[3,4-d]pyrimidin-4 (3H)-one, or a pharmaceutically acceptable salt thereof. In one embodiment, the compound is (S)-6-(2,2-dimethyl-6-(1-methyl-1H-pyrazol-4-yl)morpholino)-2,3-dimethyl-8-(2,4,5-trifluorophenyl)pyrido[3,4-d]pyrimidin-4 (3H)-one, or a pharmaceutically acceptable salt thereof. In one embodiment, the compound is (R)-8-(4-chloro-2-fluorophenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one, or a pharmaceutically acceptable salt thereof. In one embodiment, the compound is (S)-8-(4-chloro-2-fluorophenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one, or a pharmaceutically acceptable salt thereof. In one embodiment, the compound is (R)-2,3-dimethyl-6-(6-(1-methyl-1H-pyrazol-4-yl)-5-oxa-8-azaspiro[3.5]nonan-8-yl)-8-(2,4,5-trifluorophenyl)pyrido[3,4-d]pyrimidin-4 (3H)-one, or a pharmaceutically acceptable salt thereof. In one embodiment, the compound is (S)-2,3-dimethyl-6-(6-(1-methyl-1H-pyrazol-4-yl)-5-oxa-8-azaspiro[3.5]nonan-8-yl)-8-(2,4,5-trifluorophenyl)pyrido[3,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is (R)-8-(2,4-difluorophenyl)-3-methyl-6-(2-(2-methylpyridin-4-yl)morpholino)-2-(trifluoromethyl)pyrimido[5,4-d]pyrimidin-4 (3H)-one, or a pharmaceutically acceptable salt thereof. In one embodiment, the compound is (S)-8-(2,4-difluorophenyl)-3-methyl-6-(2-(2-methylpyridin-4-yl)morpholino)-2-(trifluoromethyl)pyrimido[5,4-d]pyrimidin-4 (3H)-one, or a pharmaceutically acceptable salt thereof. In one embodiment, the compound is (R)-3-methyl-6-(2-(2-methylpyridin-4-yl)morpholino)-2-(trifluoromethyl)-8-(2,4,5-trifluorophenyl)pyrimido[5,4-d]pyrimidin-4 (3H)-one, or a pharmaceutically acceptable salt thereof. In one embodiment, the compound is (S)-3-methyl-6-(2-(2-methylpyridin-4-yl)morpholino)-2-(trifluoromethyl)-8-(2,4,5-trifluorophenyl)pyrimido[5,4-d]pyrimidin-4 (3H)-one, or a pharmaceutically acceptable salt thereof. In one embodiment, the compound is (R)-6-(2,2-difluoro-6-(1-methyl-1H-pyrazol-4-yl)morpholino)-8-(2,4-difluorophenyl)-2,3-dimethylpyrimido[5,4-d]pyrimidin-4 (3H)-one, or a pharmaceutically acceptable salt thereof. In one embodiment, the compound is (S)-6-(2,2-difluoro-6-(1-methyl-1H-pyrazol-4-yl)morpholino)-8-(2,4-difluorophenyl)-2,3-dimethylpyrimido[5,4-d]pyrimidin-4 (3H)-one, or a pharmaceutically acceptable salt thereof. In one embodiment, the compound is 8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is 8-(2,4-difluorophenyl)-5-fluoro-3-methyl-6-(5-(1-methyl-1H-pyrazol-4-yl)-4-oxa-7-azaspiro[2.5]octan-7-yl)-2-(trifluoromethyl)pyrido[3,4-d]pyrimidin-4 (3H)-one, or a pharmaceutically acceptable salt thereof. In one embodiment, the compound is (R)-8-(2,4-difluorophenyl)-6-(2,2-dimethyl-6-(2-methylpyridin-4-yl)morpholino)-2,3-dimethylpyrido[3,4-d]pyrimidin-4 (3H)-one, or a pharmaceutically acceptable salt thereof. In one embodiment, the compound is (S)-8-(2,4-difluorophenyl)-6-(2,2-dimethyl-6-(2-methylpyridin-4-yl)morpholino)-2,3-dimethylpyrido[3,4-d]pyrimidin-4 (3H)-one, or a pharmaceutically acceptable salt thereof. In one embodiment, the compound is (R)-6-(2,2-dimethyl-6-(2-methylpyridin-4-yl)morpholino)-2,3-dimethyl-8-(2,4,5-trifluorophenyl)pyrido[3,4-d]pyrimidin-4 (3H)-one, or a pharmaceutically acceptable salt thereof. In one embodiment, the compound is (S)-6-(2,2-dimethyl-6-(2-methylpyridin-4-yl)morpholino)-2,3-dimethyl-8-(2,4,5-trifluorophenyl)pyrido[3,4-d]pyrimidin-4 (3H)-one, or a pharmaceutically acceptable salt thereof. In one embodiment, the compound is (R)-6-(2,2-dimethyl-6-(2-methylpyridin-4-yl)morpholino)-2,3-dimethyl-8-(6-(trifluoromethyl)pyridin-3-yl)pyrido[3,4-d]pyrimidin-4 (3H)-one, or a pharmaceutically acceptable salt thereof. In one embodiment, the compound is (S)-6-(2,2-dimethyl-6-(2-methylpyridin-4-yl)morpholino)-2,3-dimethyl-8-(6-(trifluoromethyl)pyridin-3-yl)pyrido[3,4-d]pyrimidin-4 (3H)-one, or a pharmaceutically acceptable salt thereof. In one embodiment, the compound is (R)-3-methyl-6-(5-(1-methyl-1H-pyrazol-4-yl)-4-oxa-7-azaspiro[2.5]octan-7-yl)-2-(trifluoromethyl)-8-(2,4,5-trifluorophenyl)pyrimido[5,4-d]pyrimidin-4 (3H)-one, or a pharmaceutically acceptable salt thereof. In one embodiment, the compound is (S)-3-methyl-6-(5-(1-methyl-1H-pyrazol-4-yl)-4-oxa-7-azaspiro[2.5]octan-7-yl)-2-(trifluoromethyl)-8-(2,4,5-trifluorophenyl)pyrimido[5,4-d]pyrimidin-4 (3H)-one, or a pharmaceutically acceptable salt thereof. In one embodiment, the compound is (R)-6-(2,2-dimethyl-6-(2-methylpyridin-4-yl)morpholino)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is (S)-6-(2,2-dimethyl-6-(2-methylpyridin-4-yl)morpholino)-8-(2-fluoro-4-(trifluoromethyl)phenyl)-2,3-dimethylpyrido[3,4-d]pyrimidin-4 (3H)-one, or a pharmaceutically acceptable salt thereof. In one embodiment, the compound is (R)-6-(2,2-dimethyl-6-(2-methylpyridin-4-yl)morpholino)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is (S)-6-(2,2-dimethyl-6-(2-methylpyridin-4-yl)morpholino)-2,3-dimethyl-8-(6-(trifluoromethyl)pyridin-3-yl)pyrimido[5,4-d]pyrimidin-4 (3H)-one, or a pharmaceutically acceptable salt thereof. In one embodiment, the compound is (R)-8-(2,4-difluorophenyl)-3-methyl-6-(5-(1-methyl-1H-pyrazol-4-yl)-4-oxa-7-azaspiro[2.5]octan-7-yl)-2-(trifluoromethyl)pyrimido[5,4-d]pyrimidin-4 (3H)-one, or a pharmaceutically acceptable salt thereof.

In one embodiment, the compound is (S)-8-(2,4-difluorophenyl)-3-methyl-6-(5-(1-methyl-1H-pyrazol-4-yl)-4-oxa-7-azaspiro[2.5]octan-7-yl)-2-(trifluoromethyl)pyrimido[5,4-d]pyrimidin-4 (3H)-one, or a pharmaceutically acceptable salt thereof. In one embodiment, the compound is (R)-6-(2,2-difluoro-6-(1-methyl-1H-pyrazol-4-yl)morpholino)-8-(2,4-difluorophenyl)-2,3-dimethylpyrido[3,4-d]pyrimidin-4 (3H)-one, or a pharmaceutically acceptable salt thereof. In one embodiment, the compound is (S)-6-(2,2-difluoro-6-(1-methyl-1H-pyrazol-4-yl)morpholino)-8-(2,4-difluorophenyl)-2,3-dimethylpyrido[3,4-d]pyrimidin-4 (3H)-one, or a pharmaceutically acceptable salt thereof. In one embodiment, the compound is 6-(2-(1-cyclopropyl-1H-pyrazol-4-yl)tetrahydro-2H-pyran-4-yl)-8-(2,4-difluorophenyl)-5-fluoro-3-methyl-2-(trifluoromethyl)pyrido[3,4-d]pyrimidin-4 (3H)-one, or a pharmaceutically acceptable salt thereof. In one embodiment, the compound is 8-(2-fluoro-4-(trifluoromethyl)phenyl)-2,3-dimethyl-6-((2S,4R)-2-(2-methylpyridin-4-yl)tetrahydro-2H-pyran-4-yl)pyrido[3,4-d]pyrimidin-4 (3H)-one, or a pharmaceutically acceptable salt thereof. In one embodiment, the compound is 8-(2-fluoro-4-(trifluoromethyl)phenyl)-2,3-dimethyl-6-((2R,4S)-2-(2-methylpyridin-4-yl)tetrahydro-2H-pyran-4-yl)pyrido[3,4-d]pyrimidin-4 (3H)-one, or a pharmaceutically acceptable salt thereof. In one embodiment, the compound is (R)-6-(2,2-difluoro-6-(2-methylpyridin-4-yl)morpholino)-8-(2,4-difluorophenyl)-2,3-dimethylpyrimido[5,4-d]pyrimidin-4 (3H)-one, or a pharmaceutically acceptable salt thereof. In one embodiment, the compound is (S)-6-(2,2-difluoro-6-(2-methylpyridin-4-yl)morpholino)-8-(2,4-difluorophenyl)-2,3-dimethylpyrimido[5,4-d]pyrimidin-4 (3H)-one, or a pharmaceutically acceptable salt thereof. In one embodiment, the compound is (R)-8-(2,4-difluorophenyl)-6-(2,2-dimethyl-6-(2-methylpyridin-4-yl)morpholino)-2,3-dimethylpyrimido[5,4-d]pyrimidin-4 (3H)-one, or a pharmaceutically acceptable salt thereof. In one embodiment, the compound is (S)-8-(2,4-difluorophenyl)-6-(2,2-dimethyl-6-(2-methylpyridin-4-yl)morpholino)-2,3-dimethylpyrimido[5,4-d]pyrimidin-4 (3H)-one, or a pharmaceutically acceptable salt thereof. In one embodiment, the compound is (R)-6-(2,2-difluoro-6-(2-methylpyridin-4-yl)morpholino)-8-(2,4-difluorophenyl)-2,3-dimethylpyrido[3,4-d]pyrimidin-4 (3H)-one, or a pharmaceutically acceptable salt thereof. In one embodiment, the compound is (S)-6-(2,2-difluoro-6-(2-methylpyridin-4-yl)morpholino)-8-(2,4-difluorophenyl)-2,3-dimethylpyrido[3,4-d]pyrimidin-4 (3H)-one, or a pharmaceutically acceptable salt thereof. In one embodiment, the compound is (R)-8-(4-chloro-2-fluorophenyl)-6-(2,2-dimethyl-6-(2-methylpyridin-4-yl)morpholino)-2,3-dimethylpyrido[3,4-d]pyrimidin-4 (3H)-one, or a pharmaceutically acceptable salt thereof. In one embodiment, the compound is (S)-8-(4-chloro-2-fluorophenyl)-6-(2,2-dimethyl-6-(2-methylpyridin-4-yl)morpholino)-2,3-dimethylpyrido[3,4-d]pyrimidin-4 (3H)-one, or a pharmaceutically acceptable salt thereof. In one embodiment, the compound is (R)-8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (S)-8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one, or a pharmaceutically acceptable salt thereof. In one embodiment, the compound is (R)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one, or a pharmaceutically acceptable salt thereof. In one embodiment, the compound is (S)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one, or a pharmaceutically acceptable salt thereof. In one embodiment, the compound is 8-(2,4-difluorophenyl)-2,3-dimethyl-6-((2S,6R)-2-methyl-6-(2-methylpyridin-4-yl)morpholino)pyrimido[5,4-d]pyrimidin-4 (3H)-one, or a pharmaceutically acceptable salt thereof. In one embodiment, the compound is (8-(2,4-difluorophenyl)-2,3-dimethyl-6-((2R,6S)-2-methyl-6-(2-methylpyridin-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-((2S,6R)-2-methyl-6-(2-methylpyridin-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-((2R,6S)-2-methyl-6-(2-methylpyridin-4-yl)morpholino)pyrimido[5,4-d]pyrimidin-4 (3H)-one, or a pharmaceutically acceptable salt thereof. In one embodiment, the compound is 6-((2R,6S)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one, or a pharmaceutically acceptable salt thereof. In one embodiment, the compound is 6-((2S,6R)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one, or a pharmaceutically acceptable salt thereof. In one embodiment, the compound is 6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (trans-racemate), or a pharmaceutically acceptable salt thereof. In one embodiment, the compound is 8-(4-chloro-2-fluorophenyl)-6-((2R,6S)-2-(1-cyclopropyl-1H-pyrazol-4-yl)-6-methylmorpholino)-2,3-dimethylpyrimido[5,4-d]pyrimidin-4 (3H)-one, or a pharmaceutically acceptable salt thereof. In one embodiment, the compound is 8-(4-chloro-2-fluorophenyl)-6-((2S,6R)-2-(1-cyclopropyl-1H-pyrazol-4-yl)-6-methylmorpholino)-2,3-dimethylpyrimido[5,4-d]pyrimidin-4 (3H)-one, or a pharmaceutically acceptable salt thereof. In one embodiment, the compound is 8-(2-fluoro-4-(trifluoromethyl)phenyl)-2,3-dimethyl-6-((2S,6R)-2-methyl-6-(2-methylpyridin-4-yl)morpholino)pyrimido[5,4-d]pyrimidin-4 (3H)-one, or a pharmaceutically acceptable salt thereof. In one embodiment, the compound is 8-(2-fluoro-4-(trifluoromethyl)phenyl)-2,3-dimethyl-6-((2R,6S)-2-methyl-6-(2-methylpyridin-4-yl)morpholino)pyrimido[5,4-d]pyrimidin-4 (3H)-one, or a pharmaceutically acceptable salt thereof. In one embodiment, the compound is (R)-8-(6-(difluoromethyl)pyridin-3-yl)-6-(2,2-dimethyl-6-(2-methylpyridin-4-yl)morpholino)-2,3-dimethylpyrido[3,4-d]pyrimidin-4 (3H)-one, or a pharmaceutically acceptable salt thereof. In one embodiment, the compound is (S)-8-(6-(difluoromethyl)pyridin-3-yl)-6-(2,2-dimethyl-6-(2-methylpyridin-4-yl)morpholino)-2,3-dimethylpyrido[3,4-d]pyrimidin-4 (3H)-one, or a pharmaceutically acceptable salt thereof. In one embodiment, the compound is (R)-6-(2,2-difluoro-6-(2-methylpyridin-4-yl)morpholino)-8-(6-(difluoromethyl)pyridin-3-yl)-2,3-dimethylpyrido[3,4-d]pyrimidin-4 (3H)-one, or a pharmaceutically acceptable salt thereof. In one embodiment, the compound is (S)-6-(2,2-difluoro-6-(2-methylpyridin-4-yl)morpholino)-8-(6-(difluoromethyl)pyridin-3-yl)-2,3-dimethylpyrido[3,4-d]pyrimidin-4 (3H)-one, or a pharmaceutically acceptable salt thereof. In one embodiment, the compound is 8-(2,4-difluorophenyl)-2,3-dimethyl-6-((2S,6R)-2-methyl-6-(2-methylpyridin-4-yl)morpholino)pyrido[3,4-d]pyrimidin-4 (3H)-one, or a pharmaceutically acceptable salt thereof. In one embodiment, the compound is 8-(2,4-difluorophenyl)-2,3-dimethyl-6-((2R,6S)-2-methyl-6-(2-methylpyridin-4-yl)morpholino)pyrido[3,4-d]pyrimidin-4 (3H)-one, or a pharmaceutically acceptable salt thereof. In one embodiment, the compound is 6-((2R,6S)-2-(1-cyclopropyl-1H-pyrazol-4-yl)-6-methylmorpholino)-8-(2,4-difluorophenyl)-3-methyl-2-(trifluoromethyl)pyrimido[5,4-d]pyrimidin-4 (3H)-one, or a pharmaceutically acceptable salt thereof. In one embodiment, the compound is 6-((2S,6R)-2-(1-cyclopropyl-1H-pyrazol-4-yl)-6-methylmorpholino)-8-(2,4-difluorophenyl)-3-methyl-2-(trifluoromethyl)pyrimido[5,4-d]pyrimidin-4 (3H)-one, or a pharmaceutically acceptable salt thereof. In one embodiment, the compound is 6-((2R,6S)-2-(1-cyclopropyl-1H-pyrazol-4-yl)-6-methylmorpholino)-2,3-dimethyl-8-(6-(trifluoromethyl)pyridin-3-yl)pyrido[3,4-d]pyrimidin-4 (3H)-one, or a pharmaceutically acceptable salt thereof. In one embodiment, the compound is 6-((2S,6R)-2-(1-cyclopropyl-1H-pyrazol-4-yl)-6-methylmorpholino)-2,3-dimethyl-8-(6-(trifluoromethyl)pyridin-3-yl)pyrido[3,4-d]pyrimidin-4 (3H)-one, or a pharmaceutically acceptable salt thereof. In one embodiment, the compound is (8-(2,4-difluorophenyl)-2,3-dimethyl-6-((2R,4S,6R)-2-methyl-6-(2-methylpyridin-4-yl)tetrahydro-2H-pyran-4-yl)pyrido[3,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one, or a pharmaceutically acceptable salt thereof. In one embodiment, the compound is 8-(4-chloro-2-fluorophenyl)-3-methyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is 8-(4-chloro-2-fluorophenyl)-2,3-dimethyl-6-(2-(trifluoromethyl)-5,6-dihydro-[1,2,4]triazolo[1,5-a]pyrazin-7 (8H)-yl)pyrimido[5,4-d]pyrimidin-4 (3H)-one, or a pharmaceutically acceptable salt thereof. In one embodiment, the compound is 8-(2,4-difluorophenyl)-3-methyl-6-((2S,6R)-2-methyl-6-(2-methylpyridin-4-yl)morpholino)-2-(trifluoromethyl)pyrido[3,4-d]pyrimidin-4 (3H)-one, or a pharmaceutically acceptable salt thereof. In one embodiment, the compound is 8-(2,4-difluorophenyl)-3-methyl-6-((2R,6S)-2-methyl-6-(2-methylpyridin-4-yl)morpholino)-2-(trifluoromethyl)pyrido[3,4-d]pyrimidin-4 (3H)-one, or a pharmaceutically acceptable salt thereof. In one embodiment, the compound is 6-((2R,6S)-2-(1-cyclopropyl-1H-pyrazol-4-yl)-6-methylmorpholino)-8-(2,4-difluorophenyl)-2,3-dimethylpyrido[3,4-d]pyrimidin-4 (3H)-one, or a pharmaceutically acceptable salt thereof. In one embodiment, the compound is 6-((2S,6R)-2-(1-cyclopropyl-1H-pyrazol-4-yl)-6-methylmorpholino)-8-(2,4-difluorophenyl)-2,3-dimethylpyrido[3,4-d]pyrimidin-4 (3H)-one, or a pharmaceutically acceptable salt thereof. In one embodiment, the compound is 8-(2,4-difluorophenyl)-3-methyl-6-((2S,6R)-2-methyl-6-(2-methylpyridin-4-yl)morpholino)-2-(trifluoromethyl)pyrimido[5,4-d]pyrimidin-4 (3H)-one, or a pharmaceutically acceptable salt thereof. In one embodiment, the compound is 8-(6-(difluoromethyl)pyridin-3-yl)-2,3-dimethyl-6-((2S,6R)-2-methyl-6-(2-methylpyridin-4-yl)morpholino)pyrido[3,4-d]pyrimidin-4 (3H)-one, or a pharmaceutically acceptable salt thereof. In one embodiment, the compound is 8-(6-(difluoromethyl)pyridin-3-yl)-2,3-dimethyl-6-((2R,6S)-2-methyl-6-(2-methylpyridin-4-yl)morpholino)pyrido[3,4-d]pyrimidin-4 (3H)-one, or a pharmaceutically acceptable salt thereof. In one embodiment, the compound is 6-((2R,6S)-2-(1-cyclopropyl-1H-pyrazol-4-yl)-6-methylmorpholino)-8-(2,4-difluorophenyl)-3-methyl-2-(trifluoromethyl)pyrido[3,4-d]pyrimidin-4 (3H)-one, or a pharmaceutically acceptable salt thereof. In one embodiment, the compound is 6-((2S,6R)-2-(1-cyclopropyl-1H-pyrazol-4-yl)-6-methylmorpholino)-8-(2,4-difluorophenyl)-3-methyl-2-(trifluoromethyl)pyrido[3,4-d]pyrimidin-4 (3H)-one, or a pharmaceutically acceptable salt thereof. In one embodiment, the compound is 6-((2R,6S)-2-(1-cyclopropyl-1H-pyrazol-4-yl)-6-methylmorpholino)-8-(6-(difluoromethyl)pyridin-3-yl)-2,3-dimethylpyrido[3,4-d]pyrimidin-4 (3H)-one, or a pharmaceutically acceptable salt thereof. In one embodiment, the compound is 6-((2S,6R)-2-(1-cyclopropyl-1H-pyrazol-4-yl)-6-methylmorpholino)-8-(6-(difluoromethyl)pyridin-3-yl)-2,3-dimethylpyrido[3,4-d]pyrimidin-4 (3H)-one, or a pharmaceutically acceptable salt thereof. In one embodiment, the compound is 6-((2R,6S)-2-(1-cyclopropyl-1H-pyrazol-4-yl)-6-methylmorpholino)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is 6-((2S,6R)-2-(1-cyclopropyl-1H-pyrazol-4-yl)-6-methylmorpholino)-8-(2-fluoro-4-(trifluoromethyl)phenyl)-2,3-dimethylpyrimido[5,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-8-(2,4-difluorophenyl)-3-methyl-2-(trifluoromethyl)pyrimido[5,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-8-(2,4-difluorophenyl)-3-methyl-2-(trifluoromethyl)pyrimido[5,4-d]pyrimidin-4 (3H)-one, or a pharmaceutically acceptable salt thereof. In one embodiment, the compound is 6-((2S,4R)-2-(1-cyclopropyl-1H-pyrazol-4-yl)tetrahydro-2H-pyran-4-yl)-8-(2,4-difluorophenyl)-3-methyl-2-(trifluoromethyl)pyrido[3,4-d]pyrimidin-4 (3H)-one, or a pharmaceutically acceptable salt thereof. In one embodiment, the compound is 6-((2R,4S)-2-(1-cyclopropyl-1H-pyrazol-4-yl)tetrahydro-2H-pyran-4-yl)-8-(2,4-difluorophenyl)-3-methyl-2-(trifluoromethyl)pyrido[3,4-d]pyrimidin-4 (3H)-one dimethylpyrido[3,4-d]pyrimidin-4 (3H)-one, or a pharmaceutically acceptable salt thereof. In one embodiment, the compound is 5-chloro-2-(6-(2-(1-cyclopropyl-1H-pyrazol-4-yl)-6-methylmorpholino)-2,3-dimethyl-4-oxo-3,4-dihydropyrido[3,4-d]pyrimidin-8-yl)benzonitrile, or a pharmaceutically acceptable salt thereof. In one embodiment, the compound is 2,3-dimethyl-6-((2S,6R)-2-methyl-6-(2-methylpyridin-4-yl)morpholino)-8-(6-(trifluoromethyl)pyridin-3-yl)pyrido[3,4-d]pyrimidin-4 (3H)-one, or a pharmaceutically acceptable salt thereof. In one embodiment, the compound is 2,3-dimethyl-6-((2R,6S)-2-methyl-6-(2-methylpyridin-4-yl)morpholino)-8-(6-(trifluoromethyl)pyridin-3-yl)pyrido[3,4-d]pyrimidin-4 (3H)-one, or a pharmaceutically acceptable salt thereof. In one embodiment, the compound is 4-(6-((2R,6S)-2-(1-cyclopropyl-1H-pyrazol-4-yl)-6-methylmorpholino)-2,3-dimethyl-4-oxo-3,4-dihydropyrido[3,4-d]pyrimidin-8-yl)-3-fluorobenzonitrile, or a pharmaceutically acceptable salt thereof. In one embodiment, the compound is 4-(6-((2S,6R)-2-(1-cyclopropyl-1H-pyrazol-4-yl)-6-methylmorpholino)-2,3-dimethyl-4-oxo-3,4-dihydropyrido[3,4-d]pyrimidin-8-yl)-3-fluorobenzonitrile, or a pharmaceutically acceptable salt thereof. In one embodiment, the compound is 2-(6-((2R,6S)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-fluorobenzonitrile, or a pharmaceutically acceptable salt thereof. In one embodiment, the compound is 2-(6-((2S,6R)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-fluorobenzonitrile, or a pharmaceutically acceptable salt thereof. In one embodiment, the compound is 2-(6-((2R,6S)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-(trifluoromethyl)benzonitrile, or a pharmaceutically acceptable salt thereof. In one embodiment, the compound is 2-(6-((2S,6R)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-(trifluoromethyl)benzonitrile, or a pharmaceutically acceptable salt thereof. In one embodiment, the compound is (R)-8-(4-chloro-2-fluorophenyl)-2-methyl-6-(2-(1-methyl-1H-pyrazol-4-yl)morpholino)pyrimido[5,4-d]pyrimidin-4 (3H)-one, or a pharmaceutically acceptable salt thereof. In one embodiment, the compound is (S)-8-(4-chloro-2-fluorophenyl)-2-methyl-6-(2-(1-methyl-1H-pyrazol-4-yl)morpholino)pyrimido[5,4-d]pyrimidin-4 (3H)-one, or a pharmaceutically acceptable salt thereof.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution. Tautomeric forms can also include methyltropic tautomers, which result from the swapping of a single bond with an adjacent double bond together with the concomitant migration of a methyl group.

Compounds of the invention also include all isotopes of atoms occurring in the intermediates or final compounds. Isotopes include those atoms having the same atomic number but different mass numbers. In some embodiments, the compounds of the invention include one or more isotopes of atoms in an amount greater than the natural abundance of the isotope. For example, isotopes of hydrogen include tritium and deuterium. In some embodiments, a compound of the invention includes at least one deuterium atom in an amount that is greater than the natural abundance of deuterium (e.g., the compound is enriched in deuterium).

All compounds described herein, and pharmaceutically acceptable salts thereof, can be found together with other substances such as water and solvents (e.g., in the form of hydrates and solvates).

In one aspect, the present invention is directed to an intermediate compound, or a pharmaceutically acceptable salt thereof, which can be used in the synthesis of the compounds of the present invention. For example, said intermediate compound is in some embodiments one of the intermediate compounds, or a pharmaceutically acceptable salt thereof, of any one of examples 1 to 203 disclosed herein. In some embodiments the compound of the present invention is selected from any of the intermediate compounds, or a pharmaceutically acceptable salt thereof, disclosed in any one of examples 1 to 203 herein.

The compounds of the present invention may contain, for example, one or more asymmetric carbon atoms, and therefore may exist as stereoisomers, enantiomers and diastereomers. Accordingly, the scope of the instant invention is to be understood to encompass all possible stereoisomers of the illustrated compounds, including the stereoisomerically pure form and stereoisomeric mixtures of any chemical structures disclosed herein, unless the stereochemistry is specifically identified. If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated.

The term “stereoisomer” or “stereoisomerically pure” compound as used herein refers to one stereoisomer of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

In some embodiments, the compound as defined herein is stereoisomerically pure.

Mixtures of stereoisomers may be resolved using standard techniques, such as chiral columns or chiral resolving agents, for example as outlined in the example section.

### Pharmaceutical Composition

The present invention also relates to a pharmaceutical composition comprising, for example as an active ingredient, a pharmaceutically effective amount of a compound as disclosed herein. In some embodiments, said pharmaceutical composition comprises a therapeutically effective amount of a compound as disclosed herein or a pharmaceutically acceptable salt thereof, together with at least one pharmaceutically acceptable carrier, excipient and/or diluent.

While a compound as disclosed herein for use in therapy may be administered in the form of the raw chemical compound, it is often preferred to introduce the active ingredient, optionally in the form of a pharmaceutically acceptable salt, in a pharmaceutical composition together with one or more adjuvants, excipients, carriers, buffers, diluents, and/or other customary pharmaceutical auxiliaries.

In some embodiments, the invention provides pharmaceutical compositions comprising a compound as disclosed herein or a pharmaceutically acceptable salt thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

A therapeutic amount or therapeutically effective amount or dose refers to that amount of active ingredient, i.e. the compounds or compositions as disclosed herein, which treats, alleviates, abates, or reduces the severity of symptoms of a disease in a subject, such as ameliorates one or more symptoms of the condition or the condition itself. A therapeutic amount of a compound as described herein may improve patient survival, increase survival time or rate, diminish symptoms, make an injury, disease, or condition (e.g, a neurodegenerative disease) more tolerable, slow the rate of degeneration or decline, or improve a patient's physical or mental well-being.

Therapeutic efficacy and toxicity, e.g. ED₅₀, may be determined by standard pharmacological procedures in cell cultures or experimental animals. The dose ratio between therapeutic and toxic effects is the therapeutic index and may be expressed by ratio between plasma levels resulting in therapeutic effects and plasma ratios resulting in toxic effects. Pharmaceutical compositions exhibiting large therapeutic indexes are preferred. In some embodiments, the therapeutically effective dose of a compound as disclosed herein is in the range of about 0.01 mg/kg to about 100 mg/kg bodyweight/day.

The dose administered must of course be carefully adjusted to the age, weight and condition of the individual being treated, as well as the route of administration, dosage form and regimen, and the result desired, and the exact dosage should of course be determined by the practitioner.

To administer refers to a method of delivering agents, compounds, or compositions to the desired site of biological action. These methods include, but are not limited to, enteral delivery, oral delivery, topical delivery, parenteral delivery, intravenous delivery, intradermal delivery, intramuscular delivery, intrathecal delivery, colonic delivery, rectal delivery, or intraperitoneal delivery.

### Biological Activity

As demonstrated in Example 204 compounds of the present invention are capable of modulating TREM2. Thus, in some embodiments, the compound of the present invention is a TREM2 modulator, such as a TREM2 agonist. The assay described in Example 204 may be used to assess and characterize a compound's ability to act as an agonist of TREM2. In some embodiments the compounds of the present invention are useful for the activation of TREM2. In some embodiments the compounds of the present invention activates TREM2. In some embodiments the compounds of the present invention enhances TREM2 activity. In some embodiments, a compound of the present invention induces phosphorylation of a kinase that interacts with the TREM2/DAP12 signaling complex, such as, but not limited to, Syk, ZAP70, PI3K, Erk, AKT and GSK3b. In some embodiments the compounds of the present invention enhances or activates TREM2 signaling through DAP12. In some embodiments the compounds of the present invention enhances or activates TREM2-induced phosphorylation levels of the Syk kinase. In some embodiments, a compound of the present invention induces or enhances phosphorylation of Syk if the level of Syk phosphorylation in a sample treated with the compound is increased by at least 10%, at least 20%, at least 30%, at least 40%, at least 50%, at least 60%, at least 70%, at least 80%, at least 90%, at least 100% or more as compared to a control value; such as is increased by at least 2-fold, 3-fold, 4-fold, 5-fold, 6-fold, 7-fold, 8-fold, 9-fold, 10-fold, or more as compared to a control value.

The potency of compounds of the present invention are in some embodiments expressed as EC₅₀ corresponding to the concentration of compound able to activate the phospho-Syk AlphaScreen signal to 50% of the maximal response. In some embodiments the compounds of the present invention has an EC50 value of less than 1000 nM, such as an EC50 value between 100 nM and 1000 nM, such as an EC50 value between 10 nM and 100 nM, such as an an EC50 value between 1 nM and 10 nM, such as an EC50 value<1 nM.

In some embodiments the compounds of the present invention are capable of increasing the expression of one or more TREM2 regulated genes. In some embodiments the compounds of the present invention increases the expression of one or more TREM2 regulated genes. In some embodiments the compounds of the present invention are capable or increasing one or more TREM2 regulated genes selected from the group consisting of CXCL10, CCL2, CST7 and TMEM119 (see Example 205). In some embodiments the compounds of the present invention increases expression levels, such as brain expression levels, of one or more TREM2 regulated genes selected from the group consisting of CXCL10, CCL2, CST7 and TMEM119.

In some embodiments, a compound of the present invention increases expression levels, such as brain expression levels, if the level expression of the gene in a sample treated with the compound is increased by at least 10%, at least 20%, at least 30%, at least 40%, at least 50%, at least 60%, at least 70%, at least 80%, at least 90%, at least 100% or more as compared to a control value; such as is increased by at least 1.5-fold, 2-fold, 2.5-fold, 3-fold, 3.5-fold, 4-fold, 5-fold, or more as compared to a control value (e.g. untreated control/vehicle).

### Medical Use

Being modulators of TREM2, the compounds of the present invention are of use in the treatment of diseases and disorders of a living body, including human. As used herein, the term “treatment” includes treatment, prevention, and/or alleviation or amelioration of one or more diseases and disorders or one or more symptoms of a disease or disorder. In one aspect, the compound as described herein is for use as a medicament.

In one aspect, the present invention relates to a compound, or a pharmaceutically acceptable salt thereof, as defined herein for use in the treatment of a condition associated with a loss of function of TREM2. In one aspect, the present invention relates to a compound, or a pharmaceutically acceptable salt thereof, as defined herein for use in the treatment of a condition associated with a mutation in TREM2.

In one aspect, the present invention relates to the compound, or a pharmaceutically acceptable salt thereof, as defined herein for use in the treatment of a neurodegenerative disease.

In some embodiments, a compound as described herein is used in treating a neurodegenerative disease that is characterized by a loss of function of TREM2. In some embodiments, a compound as described herein is used in treating a neurodegenerative disease that is characterized by a mutation in TREM2.

In one aspect, the present invention relates to a method for enhancing or increasing TREM2 activity in a subject in need thereof, such as in a subject having a neurodegenerative disease, said method comprising administering to the subject a compound, or a pharmaceutically acceptable salt thereof, as defined herein.

In one aspect, the present invention relates to method for one or more of i) enhancing or activating TREM2 signaling through DAP12, ii) inducing phosphorylation of a kinase that interacts with the TREM2/DAP12 signaling complex, such as, but not limited to, Syk, ZAP70, PI3K, Erk, AKT and GSK3b, iii) enhancing TREM2-induced phosphorylation levels of the Syk kinase, Iv) increasing the expression levels, such as brain expression levels, of one or more TREM2 regulated genes, and/or v) increasing the expression levels, such as brain expression levels, of one or more TREM2 regulated genes selected from the group consisting of CXCL10, CCL2, CST7 and TMEM119; in a subject in need thereof, such as in a subject having a neurodegenerative disease, said method comprising administering to the subject a compound, or a pharmaceutically acceptable salt thereof, as defined herein.

In some embodiments, the neurodegenerative disease is a tauopathy. Tautopathies depicts some neurodegenerative disorders characterized by tau deposits in the brain, with symptoms of dementia and parkinsonism. In some embodiments, the neurodegenerative disease is a tauopathy selected from the group consisting of Primary age related tauopathy (PART), globular glial tauopathy, Chronic traumatic encephalopathy (CTE), Progressive supranuclear palsy, Corticobasal degeneration, diffuse neurofibrillary tangles with calcification (DNTC), Frontotemporal dementia (FTD), and FTD with parkinsonism-17 (FTD with parkinsonism linked to chromosome 17; FTDP-17).

In some embodiments, the neurodegenerative disease is a neurodegenerative disorders associated with TDP-43 (TDP-43 proteinopathies or TDP-43-opathies). Inclusions of pathogenic deposits containing TAR DNA-binding protein 43 (TDP-43) are evident in the brain and spinal cord of patients that present across a spectrum of neurodegenerative diseases.

In some embodiments, the neurodegenerative disease is a TDP-43 proteinopathy selected from the group consisting of amyotrophic lateral sclerosis (ALS), sporadic amyotrophic lateral sclerosis (sALS), familial amyotrophic lateral sclerosis (fALS), frontotemporal lobar degeneration/disease (FTLD), Primary lateral sclerosis (PLS), progressive muscular atrophy (PMA), FTLD-tau, FTLD-FUS (bvFTLD), FTLD-TDP-43 or FTLD-U (types a, b and c), Facial onset sensory and motor neuronopathy (FOSMN), Limbic-predominant age-related TDP-43 encephalopathy (LATE), cerebral age-related TDP-43 with sclerosis (CARTS), Guam Parkinson-dementia complex (G-PDC) and ALS (G-ALS), Kii ALS/PDC, amyotrophic lateral sclerosis/parkinsonism-dementia complex of Guam (ALS-PDC), Multisystem proteinopathy (MSP; also referred to as inclusion body myopathy, IBM, associated with early-onset Paget disease of the bone and FTLD dementia), Perry disease, and disorders with concomitant TDP-43 pathology, including Alzheimer's disease (AD) and Chronic traumatic encephalopathy (CTE).

In some embodiments, the neurodegenerative disease is Multisystem proteinopathy (MSP). MSP is a dominantly inherited, pleiotropic, degenerative disorder of humans that can affect muscle, bone, and/or the central nervous system. MSP can manifest clinically as classical amyotrophic lateral sclerosis (ALS), frontotemporal dementia (FTD), inclusion body myopathy (IBM), Paget's disease of bone (PDB), or as a combination of these disorders (IBMPFD, IBMPFD/ALS).

In some embodiments, the neurodegenerative disease is a synucleinopathy. Synucleinopathies (also called α-Synucleinopathies) are neurodegenerative diseases characterised by the abnormal accumulation of aggregates of alpha-synuclein protein in neurons, nerve fibres or glial cells.

In some embodiments, the neurodegenerative disease is a synucleinopathy selected from the group consisting of Parkinson's disease (PD), dementia with Lewy bodies (DLB), multiple system atrophy (MSA), neuroaxonal dystrophies, Alzheimer's Disease with Amygdalar Restricted Lewy Bodies (AD/ALB).

In some embodiments, the neurodegenerative disease is cognitive deficit and/or memory loss. In some embodiments, the neurodegenerative disease is dementia. In some embodiments, the neurodegenerative disease is dementia selected from the group consisting of Alzheimer's disease, Parkinson's disease dementia, Huntingtons disease dementia, vascular dementia, HIV dementia, frontotemporal dementia, dementia with lewy bodies, prion disease dementia, argyrophilic grain dementia, dementia pugilistica, Guadeloupean parkinsonism with dementia, neurofibrillary tangle-predominant dementia, tangle only dementia, Down's syndrome, semantic dementia, familial British dementia, familial Danish dementia, and other dementias caused by another medical condition such as brain tumors, subdural hematoma, endocrine disorders, nutritional deficiencies, infections, immune disorders, liver or kidney failure, metabolic disorders such as Kufs disease, some leukodystrophies, some neurological disorders such as epilepsy, and multiple sclerosis.

Disorders of peripheral nerves (peripheral neuropathy) are the most common neurological complications of systemic amyloidosis. In some embodiments, the neurodegenerative disease is peripheral amyloidosis (peripheral neuropathy in systemic amyloidosis).

In some embodiments, the neurodegenerative disease is a demyelinating disorder. In some embodiments, the neurodegenerative disease is a demyelinating disorder of the central nervous system, CNS. In some embodiments, the demyelinating disorder is a myelinoclastic or demyelinating disorder, such as selected from the group consisting of multiple sclerosis, neuromyelitis optica (Devic's disease) and idiopathic inflammatory demyelinating diseases. In some embodiments, the demyelinating disorder is a leukodystrophic or dysmyelinating disorder, such as selected from the group consisting of CNS neuropathies such as vitamin B12 deficiency, central pontine myelinolysis, myelopathies such as tabes dorsalis (syphilitic myelopathy), leukoencephalopathies and leukodystrophies.

In some embodiments, the neurodegenerative disease is a demyelinating disorder of the peripheral nervous system, PNS. In some embodiments, the demyelinating disorder is selected from the group consisting of Guillain-Barré syndrome and its chronic counterpart, chronic inflammatory demyelinating polyneuropathy; Anti-MAG peripheral neuropathy; Charcot-Marie-Tooth disease and its counterpart Hereditary neuropathy with liability to pressure palsy; Copper deficiency-associated conditions (peripheral neuropathy, myelopathy, and rarely optic neuropathy); and Progressive inflammatory neuropathy.

In some embodiments, the neurodegenerative disease is Alzheimer's disease (AD). In some embodiments, the neurodegenerative disease is Alzheimer's disease (AD) with the R47H mutation. In some embodiments, the neurodegenerative disease is early Alzheimer's disease. In some embodiments, the neurodegenerative disease is Frontotemporal lobar degeneration (FTLD). In some embodiments, the neurodegenerative disease is frontotemporal dementia. In some embodiments, the neurodegenerative disease is Parkinson's disease. In some embodiments, the neurodegenerative disease is Nasu-Hakola disease (NHD). In some embodiments, the neurodegenerative disease is FTLD-like syndrome. In some embodiments, the neurodegenerative disease is Huntington disease. In some embodiments, the neurodegenerative disease is Amyotrophic lateral sclerosis (ALS). In some embodiments, the neurodegenerative disease is multiple sclerosis (MS). In some embodiments, the neurodegenerative disease is Guillain-Barre syndrome. In some embodiments, the neurodegenerative disease is chronic inflammatory demyelinating polyneuropathies. In some embodiments, the neurodegenerative disease is progressive subcortical gliosis. In some embodiments, the neurodegenerative disease is Charcot-Marie-Tooth disease. In some embodiments, the neurodegenerative disease is prion disease, such as prion protein cerebral amyloid angiopathy. In some embodiments, the neurodegenerative disease is stroke. In some embodiments, the neurodegenerative disease is cerebral amyloid angiopathy (CAA). In some embodiments the neurodegenerative disease is fragile X-associated tremor ataxia syndrome (FXTAS). In some embodiments the neurodegenerative disease is herpes simplex virus (HSV) encephalitis. In some embodiments the neurodegenerative disease is HIV-associated neurocognitive disorders (HAND). In some embodiments the neurodegenerative disease is progressive supranuclear palsy (PSP). In some embodiments the neurodegenerative disease is corticobasal degeneration. In some embodiments the neurodegenerative disease is Hallevorden-Spatz disease. In some embodiments the neurodegenerative disease is pallido-ponto-nigral degeneration. In some embodiments the neurodegenerative disease is postencephalitic parkinsonism. In some embodiments the neurodegenerative disease is subacute sclerosing panencephalitis (SSPE). In some embodiments the neurodegenerative disease is retinal degeneration (e.g., macular degeneration).

In some embodiments the neurodegenerative disease is a Leukoencephalopathy. Leukoencephalopathy (leukodystrophy-like diseases) is a term that describes all of the brain white matter diseases, whether their molecular cause is known or unknown. In some embodiments the neurodegenerative disease is a Leukoencephalopathy selected from the group consisting of Progressive multifocal leukoencephalopathy, Toxic leukoencephalopathy, Leukoencephalopathy with vanishing white matter, Leukoencephalopathy with neuroaxonal spheroids, Reversible posterior leukoencephalopathy syndrome, Megalencephalic leukoencephalopathy with subcortical cysts, and Hypertensive leukoencephalopathy. In some embodiments, the neurodegenerative disease is ALSP (Adult-onset leukoencephalopathy with axonal spheroids and pigmented glia).

In some embodiments the neurodegenerative disease is selected from the group consisting of cerebral autosomal dominant arteriopathy with subcortical infarcts or leukoencephalopathy; cerebral autosomal recessive arteriopathy with subcortical infarcts and leukoencephalopathy; and retinal vasculopathy with cerebral leukoencephalopathy (or cerebroretinal vasculopathy).

In some embodiments the neurodegenerative disease is a leukodystrophy. In some embodiments the neurodegenerative disease is vanishing white matter disease (VWM). Leukodystrophies are a group of rare, genetic disorders that affect the white matter of the brain. In some embodiments the neurodegenerative disease is a leukodystrophy selected from the group consisting of metachromatic leukodystrophy (MLD, also known as globoid cell leukodystrophy), Krabbe disease, Canavan disease, X-linked adrenoleukodystrophy, Alexander disease, hypomyelinating leukodystrophy type 7 (4H syndrome), Pelizaeus-Merzbacher disease, cerebrotendineous xanthomatosis and leukoendephalopathy with vanishing white matter. In some embodiments the neurodegenerative disease is adult-onset autosomal dominant leukodystrophy (ADLD). In some embodiments the neurodegenerative disease is X-linked adrenoleukodystrophy (X-ALD). In some embodiments the neurodegenerative disease is Nasu-Hakola disease also known as polycystic lipomembranous osteodysplasia with sclerosing leukoencephalopathy, PLOSL).

In some embodiments, the neurodegenerative disease is a transmissible spongiform encephalopathy (TSE), including Creutzfeldt-Jakob disease, Gerstmann-Straussler-Scheinker disease (GSS), kuru, and fatal familial insomnia.

In one aspect, the present invention relates to a method for treatment of a neurodegenerative disease comprising administering a compound, or a pharmaceutically acceptable salt thereof, as described herein to a subject in need thereof.

In one aspect, the present invention relates to a method for treatment of a neurodegenerative disease comprising one or more steps of administering a therapeutically effective amount of a compound, or a pharmaceutically acceptable salt thereof, as defined herein to a subject in need thereof.

In some embodiments, the subject is a mammal, such as a human.

In one aspect, the present invention relates to use of a compound, or a pharmaceutically acceptable salt thereof, as described herein for the manufacture of a medicament for treatment of a neurodegenerative disease.

In one aspect, the present invention relates to the compound, or a pharmaceutically acceptable salt thereof, as defined herein for use in the treatment of a disease or disorder associated with dysfunction of Colony stimulating factor 1 receptor (CSF1R, also known as macrophage colony-stimulating factor receptor/M-CSFR, or cluster of differentiation 115/CD115). In some embodiments the disease or disorder associated with dysfunction of CSF1R is a neurodegenerative disease associated with dysfunction of CSF1R.

In some embodiments the disease or disorder is caused by a heterozygous CSF1R mutation, a homozygous CSF1R mutation, a splice mutation in the csf1r gene, a missense mutation in the csf1 r gene, a mutation in the catalytic kinase domain of CSF1R, a mutation in an immunoglobulin domain of CSF1R, a mutation in the ectodomain of CSF1R, a loss-of-function mutation in CSF1R. In some embodiments the disease or disorder result from a change (e.g. increase, decrease or cessation) in the activity of CSF1R and/or a decrease or cessation in the activity of CSF1R.

In some embodiments the neurodegenerative disease associated with dysfunction of CSF1R is a Leukoencephalopathy.

In some embodiments the neurodegenerative disease associated with dysfunction of CSF1R is selected from the group consisting of:
adult-onset leukoencephalopathy with axonal spheroids and pigmented glia (ALSP),
hereditary diffuse leukoencephalopathy with axonal spheroids (HDLS),
pigmentary orthochromatic leukodystrophy (POLD),
pediatric-onset leukoencephalopathy,
congenital absence of microglia,
brain abnormalities neurodegeneration and dysosteosclerosis (BANDDOS), and
Cerebral autosomal dominant arteriopathy with subcortical infarcts and
leukoencephalopathy (CADASIL).

In some embodiments the neurodegenerative disease is a condition associated with dysfunction of ATP-binding cassette transporter 1 (ABCD1).

In one aspect, the present invention relates to the compound, or a pharmaceutically acceptable salt thereof, as defined herein for use in the treatment of a lysosomal storage disorder (LSD). Most lysosomal disorders cause progressive neurodegeneration leading to early death.

In some embodiments the LSD is a lipidoses, such as a lipidoses selected from the group consenting of cholesteryl ester storage disease, fucosidosis, Schindler disease and Wolman disease.

In some embodiments the LSD is a sphingolipidoses, such as a sphingolipidoses selected from the group consenting of Fabry disease, Gaucher disease, Krabbe disease (globoid cell leukodystrophy), metachromatic leukodystrophy (MLD), Niemann-Pick disease (Types A, B and C), Sandhoff disease, Farmer disease, multiple sulfatase deficiency and Tay-Sachs disease.

In some embodiments the LSD is a mucopolysaccharidoses, such as a mucopolysaccharidoses selected from the group consenting of Hunter syndrome, Hurler syndrome, Hurler-Scheie syndrome, Scheie syndrome, Sanfilippo syndrome (A, B, C, D), Morquio syndrome, Maroteaux-Lamy syndrome, Sly syndrome and Natowicz syndrome.

In some embodiments the LSD is selected from the group consisting of Batten disease, cyctinosis, Danon disease and Pompe disease.

In one aspect, the present invention relates to the compound, or a pharmaceutically acceptable salt thereof, as defined herein for use in the treatment of a disease or disorder of the bones and/or joints. In some embodiments said disease or disorder is selected from the group consisting of arthritis, rheumatoid arthritis, pyle disease, osteoporosis, osteopetrosis, osteosclerosis, skeletal dysplasia and dysosteoplasia.

In one aspect, the present invention relates to the compound, or a pharmaceutically acceptable salt thereof, as defined herein for use in the treatment of autism spectrum disorders, autism and Aspergers syndrome.

In one aspect, the present invention relates to the compound, or a pharmaceutically acceptable salt thereof, as defined herein for use in the treatment of traumatic brain injuries (TBI) and spinal cord injuries. Traumatic brain injuries (TBI), may also be known as intracranial injuries. Traumatic brain injuries occur when an external force traumatically injures the brain. Spinal cord injuries (SCI) include any injury to the spinal cord that is caused by trauma instead of disease. In some embodiments the TBI is chronic traumatic encephalopathy (CTE).

In one aspect, the present invention relates to the compound, or a pharmaceutically acceptable salt thereof, as defined herein for use in the treatment of muscular dystrophy such as myotonic dystrophy (DM) including Type 1 DM (DM1) and Type 2 DM (DM2).

In one aspect, the present invention relates to the compound, or a pharmaceutically acceptable salt thereof, as defined herein for use in the treatment of inflammation. In some embodiments said inflammation is selected from the group consisting of inclusion-body myositis, systemic lupus erythematosus (SLE), RA, gout, and certain bowel conditions including Inflammatory bowel disease (IBD).

A reduction in the functional levels of TREM2 results in dysregulation of lipid metabolism. In one aspect, the present invention relates to the compound, or a pharmaceutically acceptable salt thereof, as defined herein for use in the treatment of dysregulated lipid metabolism. In certain embodiments, the dysregulated lipid metabolism comprises increased intracellular and/or extracellular accumulation of one or more lipids. In some embodiments said dysregulated lipid metabolism is atherosclerosis.

In one aspect, the present invention relates to the compound, or a pharmaceutically acceptable salt thereof, as defined herein for use in the treatment of metabolic syndrome and conditions associated with metabolic syndrome, such as obesity, type 2 diabetes, atherosclerosis, alcoholic and non-alcoholic fatty liver disease, and alcoholic and non-alcoholic steatohepatitis,

In one aspect, the present invention relates to the compound, or a pharmaceutically acceptable salt thereof, as defined herein for use in the treatment of Amyloidosis, including AL amyloidosis (immunoglobulin light chain amyloidosis), AA amyloidosis (secondary amyloidosis), familial amyloidosis, familial systemic amyloidosis, Wild-type amyloidosis (senile systemic amyloidosis) and Localized amyloidosis.

In some embodiments, the neurodegenerative disease is Alzheimer's disease. In some embodiments, the neurodegenerative disease is Nasu-Hakola disease. In some embodiments, the neurodegenerative disease is frontotemporal dementia. In some embodiments, the method comprises administering to the subject a compound as described herein, or a pharmaceutical composition comprising a compound as described herein.

The term “compound,” as used herein is meant to include all stereoisomers, geometric isomers, tautomers, and isotopes of the structures depicted, unless otherwise specified. Accordingly, the scope of the methods and uses herein is to be understood to encompass methods and uses employing all such forms.

Depending upon the particular condition, or disease, to be treated, additional therapeutic agents, which are normally administered to treat that condition, may be administered in combination with compounds and compositions of the present invention. As used herein, additional therapeutic agents that are normally administered to treat a particular disease, or condition, are known as “appropriate for the disease, or condition, being treated.” In some embodiments, a provided combination, or composition thereof, is administered in combination with another therapeutic agent. In some embodiments, the present invention provides a method of treating a disclosed disease or condition comprising administering to a patient in need thereof an effective amount of a compound disclosed herein, or a pharmaceutically acceptable salt thereof, and co-administering simultaneously or sequentially an effective amount of one or more additional therapeutic agents. In some embodiments, the method includes co-administering one or more additional therapeutic agent. Examples of therapeutic agents the combinations of the present invention may also be combined with include, without limitation: treatments for Parkinson's disease, rheumatoid arthritis, Alzheimer's disease, Nasu-Hakola disease, frontotemporal dementia, multiple sclerosis, prion disease, or stroke. In some embodiments, the therapeutic agents the combinations of the present invention may also be combined with include, without limitation: treatments for a disease selected from the group consisting of a tauopathy, a TDP-43 proteinopathy, a synucleinopathy, dementia, amyloidosis, a demyelinating disorder of the CNS, a demyelinating disorder of the PNS, a Leukoencephalopathy, a leukodystrophy, a transmissible spongiform encephalopathy (TSE) and a lysosomal storage disorder (LSD). As used herein, the term “combination,” “combined,” and related terms refers to the simultaneous or sequential administration of therapeutic agents in accordance with the present invention. For example, a combination of the present invention may be administered with another therapeutic agent simultaneously or sequentially in separate unit dosage forms or together in a single unit dosage form.

### Methods of Manufacturing

Generally, the compounds of Formula LXI as described herein may be synthesised according to the following schemes.

All starting materials are either commercially available or known in the art and may be synthesised by using known procedures. Starting materials may also be synthesised using the procedures disclosed herein. Reaction conditions such as reaction temperature, solvent and reagents for the Schemes in this section may be found in the experimental section herein.

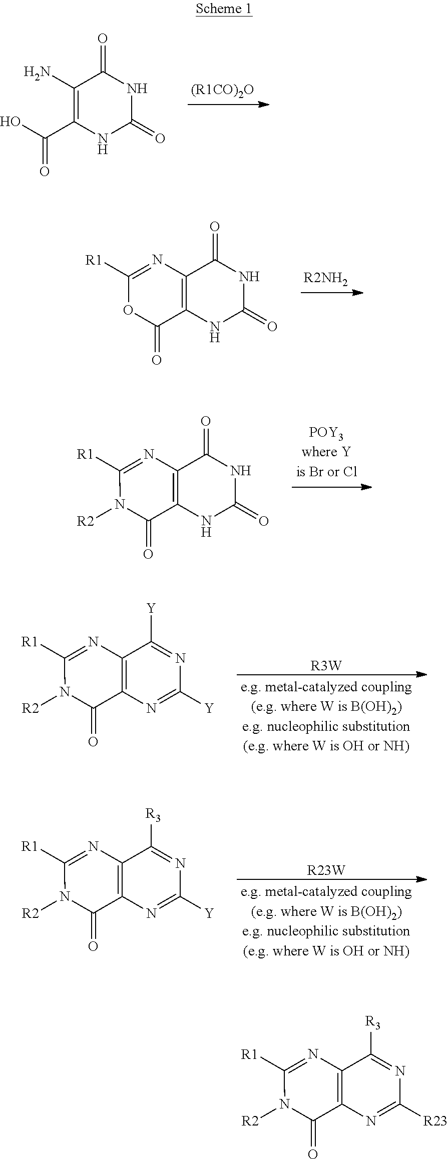

As shown in Scheme 1, (R¹CO)₂O is an anhydride that can be condensed with 5-amino-2,6-dioxo-1,2,3,6-tetrahydropyrimidine-4-carboxylic acid to form a 2-substituted pyrimido[5,4-d][1,3]oxazine-4,6,8-trione. Addition and cyclisation with R²NH₂ reagents affords the [1,3]diazino[5,4-d]pyrimidine-2,4,8-trione. Activation with POCl₃ or POBr₃ for example affords the 6,8-dihalo-pyrimido[5,4-d][1,3]diazin-4-one where Y is a Cl or Br leaving group. The R³ substituent is added via cross-coupling reaction, for example where W is a boronic acid, boronate ester or other organometallic coupling reagent such as organomagnesium, organotin or organozinc reagent. R³ may also be introduced via nucleophilic displacement of the Y leaving group. The R²³ substituent may then subsequently be introduced via either a second cross coupling reaction or second nucleophilic displacement reaction.

In one aspect, the present invention relates to a method for manufacturing a compound of formula IX as described herein, comprising the steps of:
(a) reacting a compound of formula (R¹CO)₂O with 5-amino-2,6-dioxo-1,2,3,6-tetrahydropyrimidine-4-carboxylic acid to generate a compound of formula A1

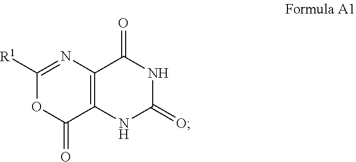

(b) reacting the compound of formula A1 generated in a) with a compound of formula R²NH₂ to generate a compound of formula A2:

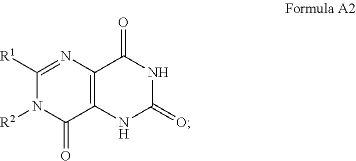

(c) activating the compound of formula A2 generated in b) with POY₃, wherein Y is Cl or Br, to generate a compound of formula A3:

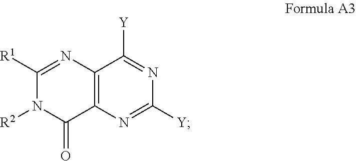

(d) reacting the compound of formula A3 generated in c) with R³W¹, wherein W is B(OH)₂, OH or NH, to generate a compound of formula A4:

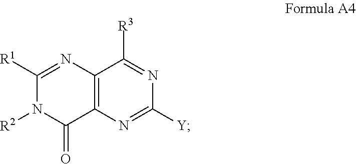

and
(e) reacting the compound of formula A4 generated in d) with R²³W², wherein W is B(OH)₂, OH or NH, to generate a compound of formula IX.

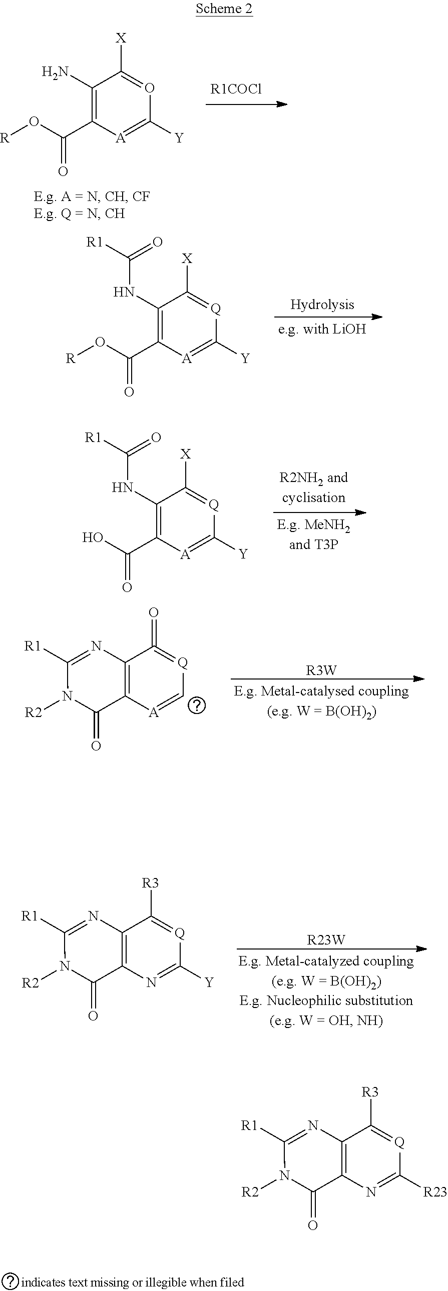

As shown in Scheme 2, R1COCl is an acid chloride that is reacted with the amino-substituted aryl starting material to form an amide. Ester hydrolysis followed by treatment with amine R²NH₂ to form an intermediate amide which then undergoes cyclisation. X and Y is a Cl or Br leaving group and can be the same or different to each other. The R³ substituent is added via cross-coupling reaction, for example where W is a boronic acid, boronate ester or other organometallic coupling reagent such as organomagnesium, organotin or organozinc reagent. R³ may also be introduced via nucleophilic displacement of the X leaving group. The R²³ substituent may then subsequently be introduced via either a second cross coupling reaction or second nucleophilic displacement reaction of the Y leaving group.

In one aspect, the present invention relates to a method for manufacturing a compound of formula B6, comprising the steps of:
(a) reacting R¹COCl with a compound of formula B1 to form a compound of formula B2:

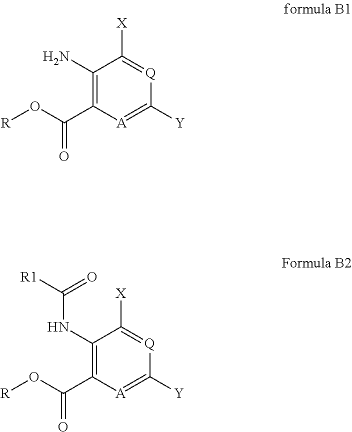

(b) hydrolysing the ester of B2 to form a compound of formula B3:

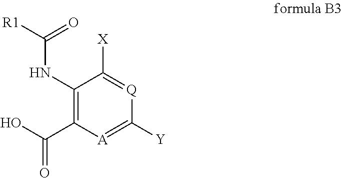

(c) reacting the compound of formula B3 R²NH₂ to form a compound of formula B4:

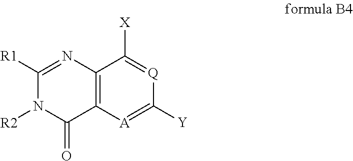

(d) reacting the compound of formula B4 with R³W¹, wherein W¹ is B(OH)₂, OH or NH, to generate a compound of formula B5:

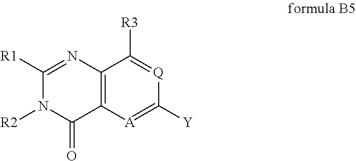

and
(e) reacting the compound of formula B5 with R²³W², wherein W² is B(OH)₂, OH or NH, to generate a compound of formula B6:

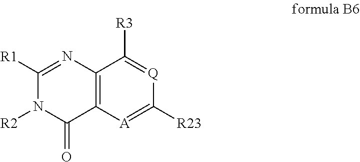

wherein R¹, R², R³ and R²³ are as defined herein and R is alkyl, such as C₁₋₆ alkyl; A is N, CH or CF; Q is N or CH; X is Cl or Br; and Y is Cl or Br.

Alternatively, as shown in Scheme 3, amino-amide substituted aryl starting materials can be cyclised in the presence of an orthoacetate reagent (e.g. R1C(OEt)₃). X and Y is a Cl or Br leaving group and can be the same or different to each other. The R³ substituent is added via cross-coupling reaction, for example where W is a boronic acid, boronate ester or other organometallic coupling reagent such as organomagnesium, organotin or organozinc reagent. R³ may also be introduced via nucleophilic displacement of the X leaving group. The R²³ substituent may then subsequently be introduced via either a second cross coupling reaction or second nucleophilic displacement reaction of the Y leaving group.

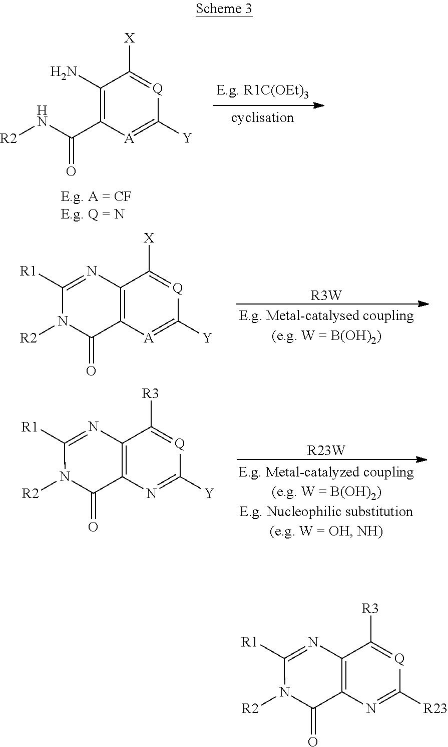

In one aspect, the present invention relates to a method for manufacturing a compound of formula C4, comprising the steps of:
(a) reacting a compound of formula C1 with an orthoacetate reagent (e.g. R¹C(OEt)₃) to form a compound of formula C2:

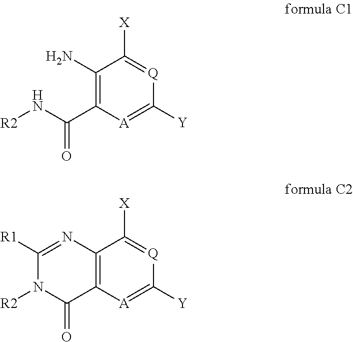

(b) reacting the compound of formula C2 with R³W¹, wherein W¹ is B(OH)₂, OH or NH, to generate a compound of formula C3:

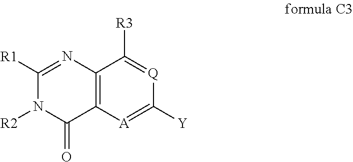

and
(c) reacting the compound of formula C3 with R²³W², wherein W² is B(OH)₂, OH or NH, to generate a compound of formula C4:

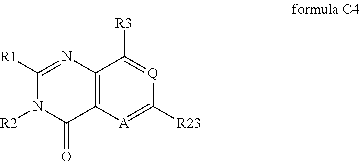

wherein R¹, R², R³ and R²³ are as defined herein and A is CH, CF or N; Q is N or CH; X is Cl or Br; and Y is Cl or Br.

In a further alternative approach, as shown in Scheme 4, R²³ substituent may be introduced via either a cross coupling reaction or nucleophilic displacement reaction of the Y leaving group. The R³ substituent is then added via a second cross-coupling reaction, for example where W is a boronic acid, boronate ester or other organometallic coupling reagent such as organomagnesium, organotin or organozinc reagent. R³ may also be introduced via a second nucleophilic displacement of the X leaving group. Subsequent reduction, hydrolysis, treatment with an anhydride (R1CO)₂O followed by cyclisation with R2NH₂ can deliver the desired compounds.

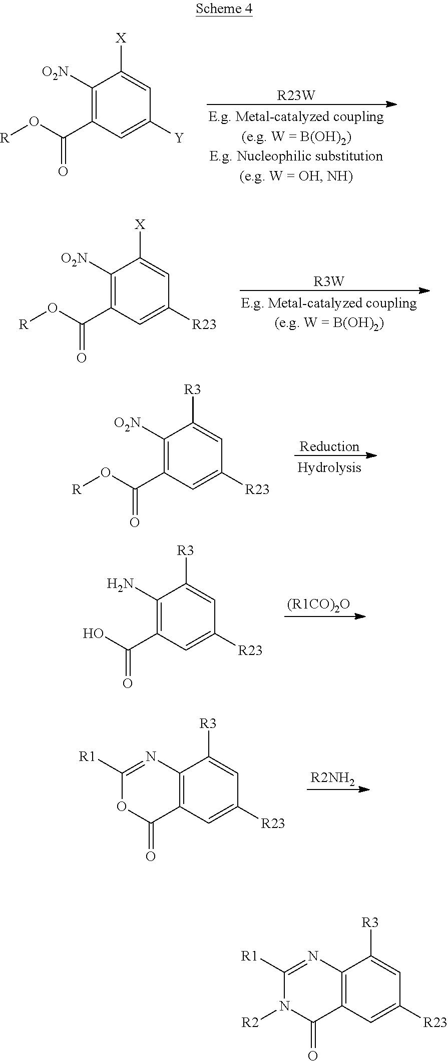

In one aspect, the present invention relates to a method for manufacturing a compound of formula D6, comprising the steps of:
(a) reacting a compound of formula D1 with R²³W², wherein W² is B(OH)₂, OH or NH, to generate a compound of formula D2:

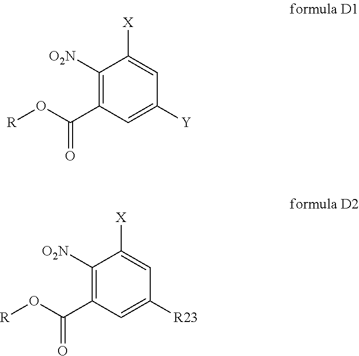

(b) reacting the compound of formula D2 with R³W¹, wherein W¹ is B(OH)₂, OH or NH, to generate a compound of formula D3:

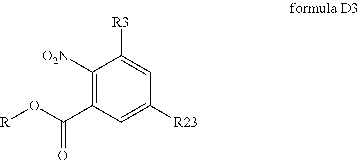

(c) reducing and hydrolysing the compound of formula D3 to generate a compound of formula D4:

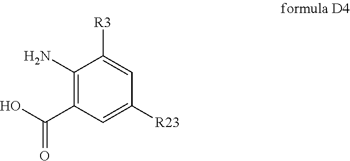

(d) reacting the compound of formula D4 with (R¹CO)₂O to generate a compound of formula D5:

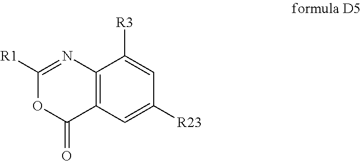

and
(e) reacting the compound of formula D5 with R²NH₂ to generate a compound of formula D6:

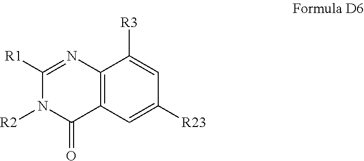

wherein R¹, R², R³ and R²³ are as defined herein and X is Cl or Br; and Y is Cl or Br, and R is alkyl, such as C₁₋₆ alkyl

As shown in Scheme 5, a metal catalysed coupling with alkyne R1CCH and a halo-substituted heterocyclic substituent can be followed with ester hydrolysis and cyclisation. Further treatment with amine R2NH₂ and R-group deprotection can afford a pyrido[3,4-d]pyrimidine-2,4,8-trione. Activation with POCl₃ or POBr₃ for example affords the 2,4-dihalo-pyrido[3,4-d]pyrimidin-8-one where X is a Cl or Br leaving group. The R³ substituent is added via cross-coupling reaction, for example where W is a boronic acid, boronate ester or other organometallic coupling reagent such as organomagnesium, organotin or organozinc reagent. R³ may also be introduced via nucleophilic displacement of the X leaving group. The R²³ substituent may then subsequently be introduced via either a second cross coupling reaction or second nucleophilic displacement reaction.

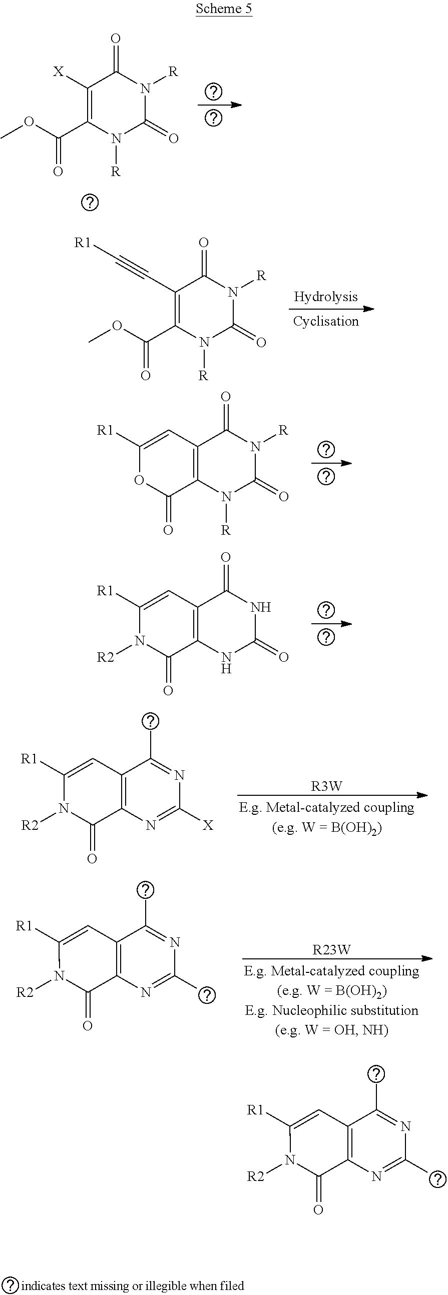

In one aspect, the present invention relates to a method for manufacturing a compound of formula E7, comprising the steps of:
(a) reacting a compound of formula E1 with R¹CCH to generate a compound of formula E2:

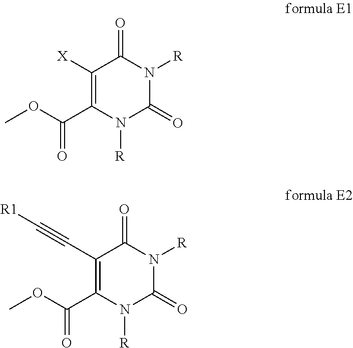

(b) hydrolysing the compound of formula E2 to generate a compound of formula E3:

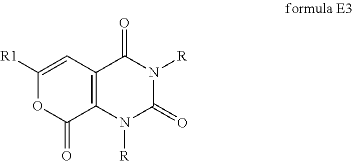

(c) reacting the compound of formula E3 with R²NH₂ and removing the R-group to generate a compound of formula E4:

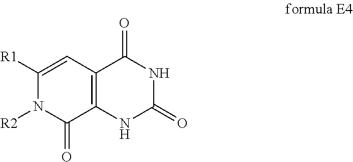

(d) reacting the compound of formula E4 with POCl₃ or POBr₃ to generate a compound of formula E5:

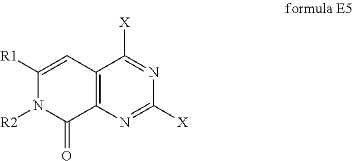

(e) reacting the compound of formula E5 with R³W¹, wherein W¹ is B(OH)₂, OH or NH, to generate a compound of formula E6:

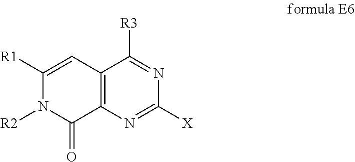

and
(f) reacting the compound of formula E6 with R²³W², wherein W² is B(OH)₂, OH or NH, to generate a compound of formula E7:

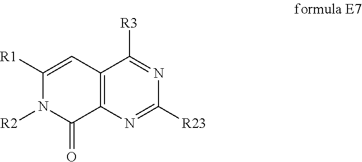

wherein R¹, R², R³ and R²³ are as defined herein and R is a suitable protecting group such as PMB; and X is I, Cl or Br.

### EXAMPLES

### List of Abbreviations

| | |
|---|---|
| ACN | acetonitrile |
| Bu | butyl |
| CataCXiumA | Di(1-adamantyl)-n-butylphosphine |
| DAST | diethylaminosulfur trifluoride |
| dba | Tris(dibenzylideneacetone |
| DBU | 1,8-Diazabicyclo(5.4.0)undec-7-ene |
| DCE | dichloroethane |
| DCM | dichloromethane |
| DIPEA | diisopropylethylamine |
| DMA | dimethylacetamide |
| DMAP | 4-dimethylaminopyridine |
| DME | 1,2-dimethoxyethane |
| DMF | dimethylformamide |
| DMS | dimethylsulfide |
| DMSO | dimethylsulfoxide |
| dppf | 1,1-bis(diphenylphosphino)ferrocene |
| EA | ethyl acetate |
| EtOH or ETOH | ethanol |
| g or gm | grams |
| HATU | 1-[Bis(dimethylamino)methylene]- |
| | 1H-1,2,3-triazolo[4,5-b]pyridinium |
| | 3-oxid hexafluorophosphate |
| HPLC | High pressure liquid chromatography |
| IPAmine | isopropylamine |
| LCMS, LC MS, | Liquid chromatography mass spectroscopy |
| LC-MS or LC/MS | |
| m/z | Mass divided by charge |
| MeNH2 | methylamine |
| MeOH or MEOH | methanol |
| mg | milligrams |
| MHz | megahertz |
| min | minutes |
| ml or mL | millilitres |
| NaH | Sodium hydride |
| NBS | N-bromosuccinimide |
| NMP | N-methylpyrrolidinone |
| NMR | nuclear magnetic resonance |
| Pd-118 | 1,1′-Bis(di-tert- |
| | butylphosphino)ferrocene]dichloropalladium(II) |
| Pd-C or Pd/C | palladium on carbon |
| PMB | 4-methoxybenzyl |
| PTSA | 4-toluenesulfonic acid |
| RT | room temperature |
| Rt | retention time |
| RuPhos | 2-Dicyclohexylphosphino-2′,6′- |
| | diisopropoxybiphenyl |
| RuPhos-Pd-G3 | (2-Dicyclohexylphosphino-2′,6′-diisopropoxy- |
| | 1,1′-biphenyl)[2-(2′-amino-1,1′- |
| | biphenyl)]palladium(II) methanesulfonate |
| SFC | Supercritical Fluid Chromatography |
| SM | starting material |
| T3P | Propanephosphonic acid anhydride |
| TEA | triethylamine |
| TFA | trifluoroacetic acid |
| THF | tetrahydrofuran |
| TMEDA | tetramethylethylenediamine |
| TMSOTf | trimethylsilyl trifluoromethanesulfonate |
| Triflic acid | 4-trifluoromethylsulfonic acid |
| xanthphos | 4,5-Bis(diphenylphosphino)-9,9-dimethylxanthene |

Certain compounds of the Examples were obtained as a mixture of stereoisomers and were subsequently separated by chiral prep HPLC. As noted in some of the Examples, the assignment of stereochemistry is arbitrary. Thus, in said Examples, the compounds obtained after the chiral prep HPLC are annotated based on the order. For example, in Example 19, two stereoisomers are obtained. These are annotated “19A”, which is peak 1, and “191B”, which is peak 2. The structure has been arbitrarily assigned to (R)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one for 19A and (S)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one for 19B. This is noted in Example 19 as “Absolute stereochemistry unknown”. However, since the assignment of stereochemistry is arbitrary (“Absolute stereochemistry unknown”), it is possible that the stereochemistry is opposite, i.e. for example 19A is (S)-6-(2-(1-cyclopropyl-1H-pyrazol-4-yl)morpholino)-2,3-dimethyl-8-(6-(trifluoromethyl) pyridin-3-yl)pyrimido[5,4-d]pyrimidin-4 (3H)-one and 19B is (R)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one. Importantly, the numbering (for example 19A for peak 1 in Example 19) is maintained in the biological evaluation.

### LCMS Conditions:

### Condition A

LCMS Column—Acquity BEH C18 (50×2.1 mm, 1.7u), Initially (90% [0.05% HCOOH in water] and 10% [0.05% HCOOH in CH₃CN:water (90:10)] is held up to 0.75 min, then to 50% [0.05% HCOOH in water] and 50% [0.05% HCOOH in CH₃CN:water (90:10)] in 1.00 min, then to 2% [0.05% HCOOH in water] and 98% [0.05% HCOOH in CH₃CN:water (90:10)] in 2.00 min held this mobile phase composition up to 2.25 min and finally back to initial condition in 2.60 min and held up to 3.00 min). Flow: 0.60 ml/min.

### Condition B

LCMS Column—YMC Triart C18 (33×2.1 mm, 3u), Initially (98% [0.05% HCOOH in water] and 2% [0.05% HCOOH in CH3CN:water (90:10)] is held up to 0.75 min, then to 90% [0.05% HCOOH in water] and 10% [0.05% HCOOH in CH₃CN:water (90:10)] in 1.00 min, then to 2% [0.05% HCOOH in water] and 98% [0.05% HCOOH in CH3CN:water (90:10)] in 2.00 min held this mobile phase composition up to 2.25 min and finally back to initial condition in 2.90 min and held up to 3.00 min). Flow: 1.00 ml/min.

### Condition C

LCMS Column—Acquity BEH C18 (50×2.1 mm, 1.7u), Initially (95% [0.05% HCOOH in water] and 5% [0.05% HCOOH in CH₃CN:water (90:10)] is held up to 0.75 min, then to 75% [0.05% HCOOH in water] and 25% [0.05% HCOOH in CH3CN:water (90:10)] in 1.50 min, then to 5% [0.05% HCOOH in water] and 95% [0.05% HCOOH in CH₃CN:water (90:10)] in 3.00 min held this mobile phase composition up to 4.00 min and finally back to initial condition in 4.50 min and held up to 5.10 min). Flow: 0.80 ml/min.

### Condition D

LCMS Column—YMC Triart C18 (33×2.1 mm, 3u), Initially (95% [0.05% HCOOH in water] and 5% [0.05% HCOOH in CH₃CN:water (90:10)] is held up to 0.75 min, then to 50% [0.05% HCOOH in water] and 50% [0.05% HCOOH in CH₃CN:water (90:10)] in 1.20 min, then to 2% [0.05% HCOOH in water] and 98% [0.05% HCOOH in CH₃CN:water (90:10)] in 2.00 min held this mobile phase composition up to 2.25 min and finally back to initial condition in 2.60 min and held up to 3.00 min). Flow: 0.60 ml/min.

### Condition E

Column—YMC TRIART C18 (33×2.1 mm, 3u), (mobile phase: 98% [0.05% HCOOH in water] and 2% [CH3CN] held for 0.75 min, then to 90% [0.05% HCOOH in water] and 10% [CH3CN] in 1.0 min. further to 2% [0.05% HCOOH in water] and 98% [CH3CN] in 2.0 min, held this mobile phase composition up to 2.25 min and finally back to initial condition in 3.0 min). Flow=1.5 ml/min

### Condition F

Column—Acquity BEH C18 (2.1×50 mm, 1.7 u) (mobile phase: 90% [0.05% HCOOH in water] and 10% [0.05% HCOOH in CH₃CN:Water (90:10)] held for 0.75 min, then to 50% [0.05% HCOOH in water] and 50% [0.05% HCOOH in CH₃CN:Water (90:10)] in 1.0 min, further to 2% [0.05% HCOOH in water] and 98% [0.05% HCOOH in CH₃CN:Water (90:10)] in 2.0 min, held this mobile phase composition up to 2.25 min and finally back to initial condition in 2.60 min and held this composition up to 3.00 min). Flow=0.60 ml/min

### Condition G

Column—Xbridge C18 (4.6×50 mm, 5 u) mobile phase: 90% [10 mM Ammonium Acetate in Water] and 10% [CH₃CN] to 70% [10 mM Ammonium Acetate in Water] and 30% [CH₃CN] in 1.5 min, further to 10% [10 mM Ammonium Acetate in Water] and 90% [CH₃CN] in 3.00 min, held this mobile phase composition up to 4.00 min and finally back to initial condition in 5.00 min. Flow=1.20 ml/min

### Condition H

Column—Acquity BEH C8 (2.1×50 mm, 1.7 u) (mobile phase: 95% [0.05% HCOOH in water] and 5% [0.05% HCOOH in CH₃CN:Water (90:10)] held for 0.75 min, then to 75% [0.05% HCOOH in water] and 25% [0.05% HCOOH in CH₃CN:Water (90:10)] in 1.5 min, further to 5% [0.05% HCOOH in water] and 95% [0.05% HCOOH in CH₃CN:Water (90:10)] in 3.00 min, held this mobile phase composition up to 4.00 min and finally back to initial condition in 4.50 min and held this composition up to 5.10 min).). Flow=0.80 ml/min

### Condition I

Column—Acquity BEH C8 (2.1×50 mm, 1.7 u) (mobile phase: 90% [0.05% HCOOH in water] and 10% [0.05% HCOOH in CH₃CN:Water (90:10)] held for 0.75 min, then to 50% [0.05% HCOOH in water] and 50% [0.05% HCOOH in CH₃CN:Water (90:10)] in 1.0 min, further to 2% [0.05% HCOOH in water] and 98% [0.05% HCOOH in CH₃CN:Water (90:10)] in 2.0 min, held this mobile phase composition up to 2.25 min and finally back to initial condition in 2.60 min and held this composition up to 3.00 min). Flow=0.80 ml/min

### Condition J

LCMS Column—Acquity BEH C8 (50×2.1 mm, 1.7u), Initially (90% [0.05% HCOOH in water] and 10% [0.05% HCOOH in CH3CN:water (90:10)] is held up to 0.75 min, then to 50% [0.05% HCOOH in water] and 50% [0.05% HCOOH in CH3CN:water (90:10)] in 1.00 min, then to 2% [0.05% HCOOH in water] and 98% [0.05% HCOOH in CH3CN:water (90:10)] in 2.00 min held this mobile phase composition up to 2.25 min and finally back to initial condition in 2.60 min and held up to 3.00 min). Flow: 0.80 ml/min.

### Condition K

LCMS Column—Acquity BEH C8 (50×2.1 mm, 1.7u), Initially (95% [0.05% HCOOH in water] and 5% [0.05% HCOOH in CH3CN:water (90:10)] is held up to 0.75 min, then to 75% [0.05% HCOOH in water] and 25% [0.05% HCOOH in CH3CN:water (90:10)] in 1.50 min, then to 5% [0.05% HCOOH in water] and 95% [0.05% HCOOH in CH3CN:water (90:10)] in 3.00 min held this mobile phase composition up to 4.00 min and finally back to initial condition in 4.50 min and held up to 5.10 min). Flow: 0.80 ml/min.

### Condition L

LCMS Column—YMC Triart C18 column (3 μm, 33×2.1 mm), Initially (95% [0.05% HCOOH in water] and 5% [0.05% HCOOH in CH3CN:water (90:10)] is held up to 0.75 min, then to 70% [0.05% HCOOH in water] and 30% [0.05% HCOOH in CH3CN:water (90:10)] in 1.00 min, then to 2% [0.05% HCOOH in water] and 98% [0.05% HCOOH in CH3CN:water (90:10)] in 2.00 min held this mobile phase composition up to 2.25 min and finally back to initial condition in 2.50 min and held up to 3.00 min). Flow: 1.00 ml/min.

### Condition M

LCMS Column—Acquity BEH C8 (50×2.1 mm, 1.7u), Initially (90% [0.05% HCOOH in water] and 10% [0.05% HCOOH in CH3CN:water (90:10)] is held up to 0.75 min, then to 50% [0.05% HCOOH in water] and 50% [0.05% HCOOH in CH3CN:water (90:10)] in 1.00 min, then to 2% [0.05% HCOOH in water] and 98% [0.05% HCOOH in CH3CN:water (90:10)] in 2.00 min held this mobile phase composition up to 2.25 min and finally back to initial condition in 2.60 min and held up to 3.00 min). Flow: 0.60 ml/min.

### Condition N

Column—Xbridge C18 (50×4.6 mm, 5u), (mobile phase: from 90% [10 mM NH4OAc in water] and 10% [CH3CN] to 70% [10 mM NH4OAc in water] and 30% [CH3CN] in 1.5 min, further to 10% [10 mM NH4OAc in water] and 90% [CH3CN] in 3.0 min, held this mobile phase composition up to 4.0 min and finally back to initial condition in 5.0 min). Flow=1.20 ml/min

### Condition O

Column—YMC Triart C18 (33×2.1 mm, 3u), (initially 98% [0.05% HCOOH in water] and 2% [0.05% HCOOH in ACN:Water (90:10)] held for 0.75 min, then to 90% [0.05% HCOOH in water] and 10% [0.05% HCOOH in ACN:Water (90:10)] in 1.0 min, further to 2% [0.05% HCOOH in water] and 98% [0.05% HCOOH in ACN:Water (90:10)] in 2.00 min, held this mobile phase composition up to 2.50 min and finally back to initial condition in 4.90 min and held this composition up to 3.0 min). Flow: 1.0 ml/min.

### Condition P

Column—Xbridge C18 column (3.5 μm, 50×3 mm), (initially 95% [5 mM NH4OAc in water] and 5% [5 mM NH4OAc in ACN:Water (90:10)] held for 0.75 min, then to 70% [5 mM NH4OAc in water] and 30% [5 mM NH4OAc in ACN:Water (90:10)] in 1.00 min, and finally 2% [5 mM NH4OAc in water] and 98% [5 mM NH4OAc in ACN:Water (90:10)] in 2.00 min, held this mobile phase composition up to 2.50 min and finally back to initial condition in 2.75 min and held this composition up to 3.0 min). Flow: 1.20 ml/min.

### Condition Q

LCMS Column—Xbridge C18 column (5 μm, 100×4.6 mm), Initially 95% [10 mM NH4OAc in water] and 5% [ACN] held for 1.50 min, then 60% [10 mM NH4OAc in water] and 40% [ACN] in 5.00 min further 5% [10 mM NH4OAc in water] and 95% [ACN] in 2.50 min, and finally 2% [10 mM NH4OAc in water] and 98% ACN in 9.50 min, held this mobile phase composition up to 12.50 min and finally back to initial condition in 14.00 min and held this composition up to 15.00 min. Flow: 1.00 ml/min.

### Condition R

Column—Gemini NX C18 (4.6×100 mm, 3 u) mobile phase: 98% [10 mM Ammonium Acetate in Water] and 2% [ACN] held for 0.50 min then 50% [10 mM Ammonium Acetate in Water] and 50% [ACN] in 6.50 min, further 5% [10 mM Ammonium Acetate in Water] and 95% [ACN] in 9.50 min, held this mobile phase composition up to 13.00 min and finally back to initial condition in 14.00 min and held this composition up to 15.00 min. Flow=1.00 ml/min

### Example 1

### 8-(4-chloro-2-fluorophenyl)-2,3-dimethyl-6-(2-(1-methyl-1H-pyrazol-4-yl)morpholino)pyrimido[5,4-d]pyrimidin-4 (3H)-one

### Example 1A: Enantiomer Peak 1, Example 1B: Enantiomer Peak 2

### Step-1—Preparation of 2-methyl-4H-pyrimido[5,4-d][1,3]oxazine-4,6,8 (5H,7H)-trione

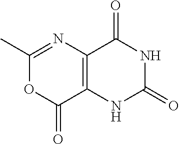

To a stirred solution of 5-amino-2,6-dioxo-1,2,3,6-tetrahydropyrimidine-4-carboxylic acid (2 g, 11.70 mmol) in acetic anhydride (20.17 mL, 213.45 mmol) was added pyridine (3.96 mL, 49.12 mmol) drop wise at RT. Resulting mixture was heated at 120° C. for 2 h. It was cooled to ambient temperature and concentrated under reduced pressure. Crude mass was diluted with chloroform and again concentrated under reduced pressure. The residue thus obtained was triturated with diethyl ether, acetonitrile and dried to afford 2-methyl-4H-pyrimido[5,4-d][1,3]oxazine-4,6,8 (5H,7H)-trione (2 g, 87.63% yield) as brown solid.

1H NMR (400 MHz, DMSO D6) δ 11.7 (1H, s), 11.55 (1H, s), 2.34 (3H, s)

LCMS Condition A: Rt=0.28 min. m/z 196.1 [M+H]⁺.

### Step-2—Preparation of 6,7-dimethyl-1,7-dihydropyrimido[5,4-d]pyrimidine-2,4,8 (3H)-trione

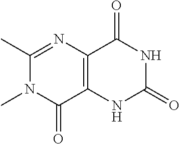

To a stirred solution of 2-methyl-4H-pyrimido[5,4-d][1,3]oxazine-4,6,8 (5H,7H)-trione (2 g, 10.26 mmol) in acetic acid (20 mL) was added sodium acetate (840 mg, 10.26 mmol) followed by drop wise addition of MeNH2 [2(M) THF solution, 5.13 mL, 10.26 mmol] at RT. Resulting mixture was heated at 120° C. for 16 h. The mixture was cooled to RT and concentrated under reduced pressure. The residue was diluted with water and precipitate thus formed was collected and dried. It was then triturated with diethyl ether, ethyl acetate successively and dried under high vacuum to afford 6,7-dimethyl-1,7-dihydropyrimido[5,4-d]pyrimidine-2,4,8 (3H)-trione (1.35 g, 63.2% yield) as light brown solid.

1H NMR (400 MHz, DMSO D6) δ 11.51 (1H, s), 10.98 (1H, s), 3.50 (3H, s), 2.50 (3H, s)

LCMS Condition B: Rt=0.62 min. m/z 209.38 [M+H]⁺.

### Step-3—Preparation of 6,8-dichloro-2,3-dimethylpyrimido[5,4-d]pyrimidin-4 (3H)-one

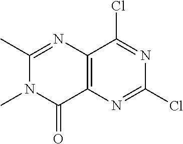

To a stirred solution of 6,7-dimethyl-1,7-dihydropyrimido[5,4-d]pyrimidine-2,4,8 (3H)-trione (600 mg, 2.88 mmol) in POCl3 (12.028 mL, 128.654 mmol), DIPEA (2.01 mL, 11.538 mmol) was added drop wise at 0° C. Resultant mixture was stirred at RT for 15 min and then heated to 100° C. for 16 h. The mixture was cooled to RT and concentrated under reduced pressure. The residue was cooled to 0° C., quenched with saturated aqueous NaHCO₃ solution and extracted with ethyl acetate. Combined organic part was washed with water, brine, dried over anhydrous sodium sulphate, filtered and concentrated under reduced pressure. Crude mass was purified by column chromatography (50% ethyl acetate-hexane) to afford 6,8-dichloro-2,3-dimethylpyrimido[5,4-d]pyrimidin-4 (3H)-one (400 mg, 56.58% yield) as yellow solid.

1H NMR (400 MHz, DMSO D6) δ 3.56 (3H, s), 2.66 (3H, s)

LCMS Condition A: Rt=1.68 min. m/z 245.2 [M+H]⁺.

### Step-4—Preparation of 6-chloro-8-(4-chloro-2-fluorophenyl)-2,3-dimethylpyrimido[5,4-d]pyrimidin-4 (3H)-one

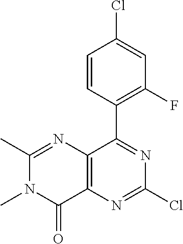 [SMILES: Cc1nc2c(-c3ccc(Cl)cc3F)nc(Cl)nc2c(=O)n1C]

To a stirred solution of 6,8-dichloro-2,3-dimethylpyrimido[5,4-d]pyrimidin-4 (3H)-one (200 mg, 0.816 mmol) and (4-chloro-2-fluorophenyl)boronic acid (127.83 mg, 0.735 mmol) in dioxane (6 mL) and water (2 mL) was added sodium carbonate (128.571 mg, 1.224 mmol) and degassed with argon. PdCl2(dppf) (59.673 mg, 0.082 mmol) was added under inert atmosphere. The resulting mixture was heated at 90° C. for 16 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (35% ethyl acetate-hexane) to afford 6-chloro-8-(4-chloro-2-fluorophenyl)-2,3-dimethylpyrimido[5,4-d]pyrimidin-4 (3H)-one (200 mg, 72% yield). LCMS: m/z 339.2 [M+H]⁺.

1H NMR (400 MHz, DMSO D6) δ 7.72-7.68 (2H, m), 7.52 (1H, d, J=8.32 Hz), 3.56 (3 H, s), 2.54 (3H, s)

LCMS Condition A: Rt=2.14 min. m/z 339.2 [M+H]⁺.

### Step-5—Preparation of 8-(4-chloro-2-fluorophenyl)-2,3-dimethyl-6-(2-(1-methyl-1H-pyrazol-4-yl)morpholino)pyrimido[5,4-d]pyrimidin-4 (3H)-one

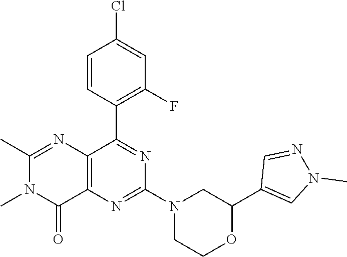

To a stirred solution of 2-(1-methyl-1H-pyrazol-4-yl)morpholine (HCl salt, 141.593 mg, 0.59 mmol) in DMSO (2 mL) was added DIPEA (0.206 mL, 1.18 mmol) at RT and stirred for 15 min. To it was added 6-chloro-8-(4-chloro-2-fluorophenyl)-2,3-dimethylpyrimido[5,4-d]pyrimidin-4 (3H)-one (100 mg, 0.295 mmol) at RT.

Resulting mixture was heated at 100° C. for 16 h. Reaction mixture was concentrated under reduced pressure. Crude mass was purified by reverse phase preparative HPLC to afford 8-(4-chloro-2-fluorophenyl)-2,3-dimethyl-6-(2-(1-methyl-1H-pyrazol-4-yl)morpholino)pyrimido[5,4-d]pyrimidin-4 (3H)-one (40 mg, 60.94 mmol, 29% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 Mm Ammonium bicarbonate in water, B=Acetonitrile; Gradient Profile: Mobile phase initial composition of 60% A and 40% B, then 40% A and 60% B in 3 min, then to 20% A and 80% B in 20 min., then to 5% A and 95% B in 21 min., held this composition up to 22 min. for column washing, then returned to initial composition in 23 min. and held till 25 min.

1H NMR (400 MHz, DMSO D6) δ 7.74 (1H, s) 7.68-7.64 (1H, m) 7.61-7.59 (1H, m), 7.45-7.44 (2H, m), 4.63-4.60 (1H, m), 4.52-4.49 (2H, m), 4.01-3.99 (1H, m), 3.81 (3H, s), 3.66-3.63 (1H, m), 3.51 (3H, s), 3.18-3.12 (2H, m), 2.44 (3H, s).

LCMS Condition C: Rt=2.86 min. m/z 470.27 [M+H]⁺.

### Step-6—Preparation of (R)-8-(4-chloro-2-fluorophenyl)-2,3-dimethyl-6-(2-(1-methyl-1H-pyrazol-4-yl)morpholino)pyrimido[5,4-d]pyrimidin-4 (3H)-one [Example 1A] and (S)-8-(4-chloro-2-fluorophenyl)-2,3-dimethyl-6-(2-(1-methyl-1H-pyrazol-4-yl)morpholino)pyrimido[5,4-d]pyrimidin-4 (3H)-one [Example 1B]

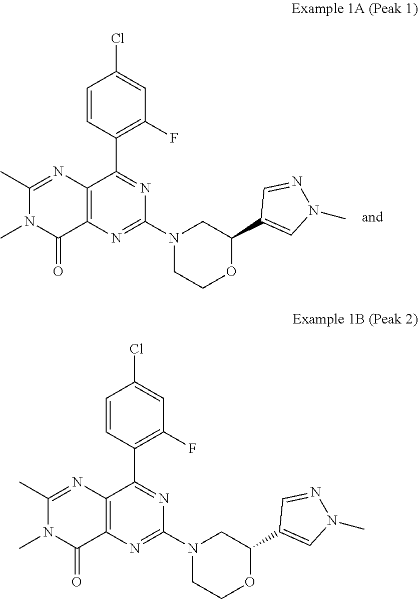

Chiral separation of 8-(4-chloro-2-fluorophenyl)-2,3-dimethyl-6-(2-(1-methyl-1H-pyrazol-4-yl)morpholino) pyrimido[5,4-d]pyrimidin-4 (3H)-one (30 mg, 0.064 mmol) was done by normal phase chiral prep HPLC to afford Example 1A, (R)-8-(4-chloro-2-fluorophenyl)-2,3-dimethyl-6-(2-(1-methyl-1H-pyrazol-4-yl)morpholino)pyrimido[5,4-d]pyrimidin-4 (3H)-one (8.9 mg, 29.6% yield; eluted first, assigned as Peak 1 with arbitrary assignment of stereochemistry) as light yellow solid and Example 1B (S)-8-(4-chloro-2-fluorophenyl)-2,3-dimethyl-6-(2-(1-methyl-1H-pyrazol-4-yl)morpholino)pyrimido[5,4-d]pyrimidin-4 (3H)-one (9.6 mg, 3.95 mmol, 31.9% yield, eluted later, assigned as Peak 2 with arbitrary assignment of stereochemistry) as light yellow solid.

Chiral prep HPLC method details to separate the enantiomers:

Instrument: Agilent 1200 series instrument/Column: CHIRALPAK IC (250×21 mm) 5u/Flow rate: 21.0 ml/min/Mobile phase: Hexane/Dichloromethane/Ethanol: 65/17.5/17.5/Solubility: Mixture of Acetonitrile and Dichloromethane/Wave length: 295 nm/Run time: 36 min

Example 1A (Peak 1): HNMR: 1H NMR (400 MHz, DMSO D6) δ 7.74 (1H, s) 7.67 (1H, t, J=7.96 Hz) 7.62-7.59 (1H, m), 7.46-7.44 (2H, m), 4.62 (1H, br d, J=13.08 Hz), 4.52-4.59 (2H, m), 4.01-3.99 (1H, m), 3.81 (3H, s), 3.66-3.65 (1H, m), 3.51 (3H, s), 3.18-3.12 (2H, m), 2.44 (3H, s).

LCMS Condition D: Rt=1.87 min. m/z 470 [M+H]⁺.

Example 1B (Peak 2) HNMR: 1H NMR (400 MHz, DMSO D6) δ 7.74 (1H, s) 7.67 (1H, t, J=7.96 Hz) 7.62-7.59 (1H, m), 7.45-7.44 (2H, m), 4.62 (1H, br d, J=13.08 Hz), 4.52-4.59 (2H, m), 4.01-3.99 (1H, m), 3.81 (3H, s), 3.66-3.65 (1H, m), 3.51 (3H, s), 3.18-3.12 (2H, m), 2.44 (3H, s).

LCMS Condition D: Rt=3.03 min, m/z 470.3 [M+H]⁺.

Analytical chiral HPLS: Chiralpak IC (4.6×250 mm), 5μ Mobile Phase: Hexane/DCM/EtOH/IPAmine: 60/20/20/0.1 Flow Rate: 1.0 ml/min. Solubility: MeOH, Rt=12.62 min for Peak 1 and Rt=14.95 min for Peak 2.

### Example 2

### 8-(4-chloro-2-fluorophenyl)-6-(2-(1-cyclopropyl-1H-pyrazol-4-yl)morpholino)-2,3-dimethylpyrimido[5,4-d]pyrimidin-4 (3H)-one

### Step-1—Preparation of 1-benzyl-4-bromo-1H-pyrazole

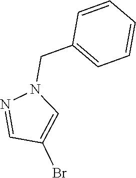 [SMILES: Brc1cnn(Cc2ccccc2)c1]

To a stirred solution of 4-bromo-1H-pyrazole (5 g, 34.02 mmol) in DMF (60 mL) at 0° C. was added sodium hydride (60% in oil, 2.04 g, 51.03 mmol) in portions. Resulting mixture was stirred at RT for 0.5 h. Benzyl bromide (6.06 mL, 51.03 mmol) was added dropwise at 0° C. Resulting solution was stirred at RT for 16 h. Reaction mixture was cooled to 0° C., quenched with crushed ice and extracted with ethyl acetate. Combined organic layer was washed with water, brine, dried over sodium sulphate, filtered and evaporated under reduced pressure. Crude product was purified by combiflash chromatography (20-30% ethyl acetate-hexane) to afford 1-benzyl-4-bromo-1H-pyrazole (7.5 g, 92.9% yield) as colorless liquid.

1H NMR (400 MHz, DMSO-d6) δ 8.09 (s, 1H), 7.57 (s, 1H), 7.36-7.23 (m, 5H), 5.31 (s, 2H).

LCMS Condition F: Rt=2.07 min. m/z 237.1 [M+H]⁺.

### Step-2—Preparation of 1-benzyl-4-(1-ethoxyvinyl)-1H-pyrazole

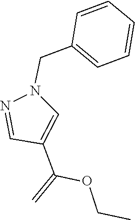 [SMILES: C=C(OCC)c1cnn(Cc2ccccc2)c1]

To a stirred solution of 1-benzyl-4-bromo-1H-pyrazole (3.3 g, 13.92 mmol) in toluene (70 mL) was added tributyl(1-ethoxyvinyl)stannane (5.2 mL) Resulting mixture was degassed with argon and Pd(PPh₃)₄ (805 mg, 0.69 mmol) was added under inert atmosphere. Resulting mixture was heated to 110° C. for 16 h. Reaction mixture was filtered through a short pad of celite and washed with ethyl acetate. Combined filtrate was concentrated under reduced pressure to afford 1-benzyl-4-(1-ethoxyvinyl)-1H-pyrazole (3.1 g, crude) as black liquid. The crude material was used in the next step without further purification.

### Step-3—Preparation of 1-(1-benzyl-1H-pyrazol-4-yl)-2-bromoethan-1-one

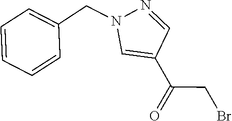 [SMILES: O=C(CBr)c1cnn(Cc2ccccc2)c1]

To a stirred solution of 1-benzyl-4-(1-ethoxyvinyl)-1H-pyrazole (3.15 g, 13.82 mmol) in THF (90 mL) and water (25 mL) at 0° C. was added NBS (2.95 g, 16.58 mmol). The resulting solution was stirred at RT for 1 h. Reaction mixture was quenched with water and extracted with ethyl acetate. The combined organic layers were washed with water, brine, dried over anhydrous sodium sulphate, filtered and concentrated under reduced pressure. Crude mass was purified by column chromatography (25-40% ethyl acetate-hexane) to afford 1-(1-benzyl-1H-pyrazol-4-yl)-2-bromoethan-1-one (960 mg, 24.3% yield) as colorless liquid.

1H NMR (400 MHz, DMSO-d6) δ 8.64 (s, 1H), 8.04 (s, 1H), 7.36-7.28 (m, 5H), 5.39 (s, 2H), 4.59 (s, 2H).

LCMS Condition F: Rt=1.91 min. m/z 279.2 [M+H]⁺.

### Step-4—Preparation of 2-(benzyl(2-hydroxyethyl)amino)-1-(1-benzyl-1H-pyrazol-4-yl)ethan-1-one

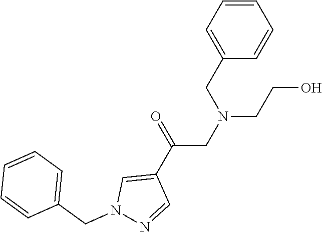 [SMILES: O=C(CN(CCO)Cc1ccccc1)c1cnn(Cc2ccccc2)c1]

To a stirred solution of 1-(1-benzyl-1H-pyrazol-4-yl)-2-bromoethan-1-one (940 mg, 3.37 mmol) in acetonitrile (20 mL) were added potassium carbonate (1.16 g, 8.42 mmol) and 2-(benzylamino)ethan-1-ol (661.4 mg, 4.38 mmol) at RT. Resulting mixture was heated to 70° C. for 4 h. Reaction mixture was quenched with water and extracted with ethyl acetate. The combined organic layer was washed with water, brine, dried over anhydrous sodium sulphate, filtered and concentrated under reduced pressure. Crude mass was purified by combiflash chromatography (50-80% ethyl acetate in hexane) to afford 2-(benzyl(2-hydroxyethyl)amino)-1-(1-benzyl-1H-pyrazol-4-yl)ethan-1-one (1.1 g, 93.4% yield) as yellow liquid.

1H NMR (400 MHz, DMSO-d6) δ 8.57 (s, 1H), 7.96 (s, 1H), 7.37-7.23 (m, 10H), 5.36 (s, 2H), 4.44 (t, J=5.12 Hz, 1H), 3.69-3.68 (m, 4H), 3.51-3.46 (m, 2H), 2.61-2.58 (m, 2H).

LCMS Condition E: Rt=1.51 min. m/z 350.41 [M+H]⁺.

### Step-5—Preparation of 2-(benzyl(2-hydroxyethyl)amino)-1-(1-benzyl-1H-pyrazol-4-yl)ethan-1-ol

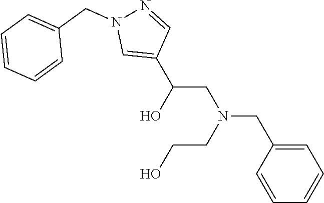 [SMILES: OCCN(Cc1ccccc1)CC(O)c1cnn(Cc2ccccc2)c1]

To a stirred solution of 2-(benzyl(2-hydroxyethyl)amino)-1-(1-benzyl-1H-pyrazol-4-yl)ethan-1-one (500 mg, 1.43 mmol) in methanol (10 mL) sodium borohydride (108.4 mg, 2.86 mmol) was added portion wise at 0° C. Resulting mixture was stirred at 0° C. for 0.5 h and then at RT for 2 h. Reaction mixture was quenched with ice cold water and extracted with dichloromethane. Combined organic layer was washed with water, brine, dried over anhydrous sodium sulphate, filtered and concentrated under reduced pressure. Crude mass was triturated with diethyl ether to afford 2-(benzyl(2-hydroxyethyl)amino)-1-(1-benzyl-1H-pyrazol-4-yl)ethan-1-ol (470 mg, 93.3% yield) as colorless sticky solid.

1H NMR (400 MHz, DMSO-d6) δ 7.62 (s, 1H), 7.33-7.19 (m, 11H), 5.25 (s, 2H), 4.81 (d, J=3.68 Hz, 1H), 4.63-4.61 (m, 1H), 4.37 (t, J=5.04 Hz, 1H), 3.71-3.64 (m, 2H), 3.46-3.78 (m, 2H), 2.64-2.55 (m, 4H).

LCMS Condition F: Rt=1.61 min. m/z 352.4 [M+H]⁺.

### Step-6—Preparation of 4-benzyl-2-(1-benzyl-1H-pyrazol-4-yl)morpholine, HCl salt

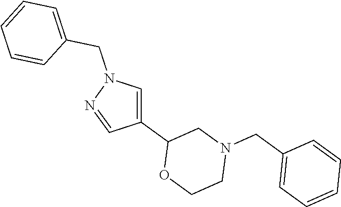 [SMILES: c1ccc(CN2CCOC(c3cnn(Cc4ccccc4)c3)C2)cc1]

6(N) HCl (5.0 mL) was added to 2-(benzyl(2-hydroxyethyl)amino)-1-(1-benzyl-1H-pyrazol-4-yl)ethan-1-ol (470 mg, 1.34 mmol). Resulting mixture was heated at 110° C. for 2 h. Reaction mixture was concentrated under reduced pressure, triturated with diethyl ether and dried to afford 4-benzyl-2-(1-benzyl-1H-pyrazol-4-yl)morpholine (430.0 mg, 96.3% yield, HCl salt) as a gum.

1H NMR (400 MHz, DMSO-d6) δ 11.6 (s, 1H), 7.87 (s, 1H), 7.63-7.61 (m, 2H), 7.48-4.46 (m, 4H), 7.35-7.28 (m, 3H), 7.22-7.20 (m, 2H), 5.30 (s, 2H), 4.93-4.91 (m, 2H), 4.34-4.32 (m, 2H), 4.01-3.98 (m, 2H), 3.39 (d, J=11.56 Hz, 1H), 3.23-3.20 (m, 1H), 3.16-3.10 (m, 1H).

LCMS Condition F: Rt=1.67 min. m/z 334.4 [M+H]⁺.

### Step-7—Preparation of 2-(1H-pyrazol-4-yl)morpholine

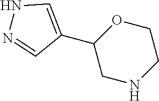 [SMILES: c1n[nH]cc1C1CNCCO1]

To a degassed solution of 4-benzyl-2-(1-benzyl-1H-pyrazol-4-yl)morpholine (500 mg, 1.23 mmol, HCl salt) in ethanol (20 mL), palladium hydroxide (150 mg, 0.25 mmol) was added. The resulting mixture was hydrogenated in Parr Shaker under 30 psi pressure for 16 h at RT. Reaction mixture was filtered through a short pad of celite and washed with ethanol. Combined filtrate was concentrated under reduced pressure to afford 2-(1H-pyrazol-4-yl)morpholine (180 mg, 95.6% yield, HCl salt) as a gum.

1H NMR (400 MHz, DMSO-d6) δ 9.74 (bs, 1H), 9.61 (bs, 1H), 7.71 (s, 2H), 4.79 (d, J=9.24 Hz, 1H), 4.01-3.98 (m, 1H), 3.93-3.87 (m, 2H), 3.36-3.33 (m, 1H), 3.21-3.16 (m, 2H), 3.10-2.99 (m, 2H).

LCMS Condition F: Rt=0.23 min. m/z 154.2 [M+H]⁺.

### Step-8—Preparation of tert-butyl 2-(1H-pyrazol-4-yl)morpholine-4-carboxylate

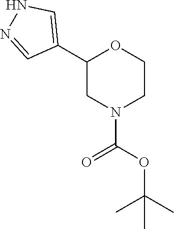 [SMILES: CC(C)(C)OC(=O)N1CCOC(c2cn[nH]c2)C1]

To a stirred solution of 2-(1H-pyrazol-4-yl)morpholine, HCl salt (300 mg, 1.33 mmol) in dioxane (3 ml) and water (1 mL) were added triethyl amine (0.37 mL, 2.67 mmol), 4-Dimethylaminopyridine (8.1 mg, 0.07 mmol) and a solution of di-tert-butyl dicarbonate (0.15 mL, 0.67 mmol) in dioxane (2 mL). Resulting mixture was stirred at RT for 16 h. Reaction mixture was quenched with water and extracted with ethyl acetate. Combined organic layer was washed with water, brine, dried over anhydrous sodium sulphate, filtered and concentrated under reduced pressure. Crude mass was purified under column chromatography (60-70% ethyl acetate in hexane) to afford tert-butyl 2-(1H-pyrazol-4-yl)morpholine-4-carboxylate (190 mg, 56.3% yield) as white solid.

1H NMR (400 MHz, DMSO) δ 12.79 (s, 1H), 7.72 (s, 1H), 7.47 (s, 1H), 4.38-4.35 (m, 1H), 3.85-3.72 (m, 3H), 3.51-3.46 (m, 1H), 2.95 (bs, 2H), 1.41 (s, 9H).

LCMS Condition G: Rt=2.63 min. m/z 254.1 [M+H]⁺.

### Step-9—Preparation of tert-butyl 2-(1-cyclopropyl-1H-pyrazol-4-yl)morpholine-4-carboxylate

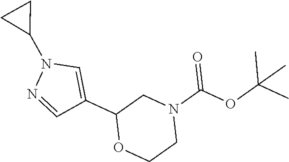 [SMILES: CC(C)(C)OC(=O)N1CCOC(c2cnn(C3CC3)c2)C1]

To a stirred solution of tert-butyl 2-(1-cyclopropyl-1H-pyrazol-4-yl)morpholine-4-carboxylate (190 mg, 0.75 mmol) and cyclopropyl boronic acid (141.92 mg, 1.65 mmol) in dichloroethane (4 mL) was added sodium carbonate (174.9 mg, 1.65 mmol).

Resulting solution was purged with oxygen for 10 min and 2,2′-bipyridine (129.03 mg, 0.83 mmol) and copper acetate (150.04 mg, 0.83 mmol) were added. Resulting mixture was heated at 70° C. for 16 h under oxygen atmosphere. Reaction mixture was filtered through a short pad of celite, washed with dichloromethane. Combined filtrate was washed with 2N HCl, water, brine, dried over anhydrous sodium sulphate, filtered and concentrated under reduced pressure. Crude mass was purified by column chromatography (20-50% ethyl acetate in hexane) to afford tert-butyl 2-(1-cyclopropyl-1H-pyrazol-4-yl)morpholine-4-carboxylate (100.0 mg, 45.3% yield) as colorless gum.

1H NMR (400 MHz, DMSO) δ 7.76 (s, 1H), 7.39 (s, 1H), 4.33-4.30 (m, 1H), 3.83-3.81 (m, 2H), 3.74-3.65 (m, 2H), 3.51-3.45 (m, 1H), 2.91 (bs, 2H), 1.41 (s, 9H), 0.99-0.98 (m, 2H), 0.94-0.92 (m, 2H).

LCMS Condition G: Rt=2.99 min. m/z 294.0 [M+H]⁺.

### Step-10—Preparation of 2-(1-cyclopropyl-1H-pyrazol-4-yl)morpholine

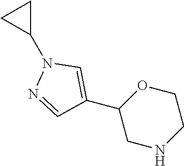 [SMILES: c1nn(C2CC2)cc1C1CNCCO1]

To a stirred solution of tert-butyl 2-(1-cyclopropyl-1H-pyrazol-4-yl)morpholine-4-carboxylate (100 mg, 0.34 mmol) in dichloromethane (3 mL) at 0° C. was added TFA (2 mL). The resulting mixture was stirred at RT for 3 h. Reaction mixture was concentrated under reduced pressure, triturated with diethyl ether, and dried to afford 2-(1-cyclopropyl-1H-pyrazol-4-yl)morpholine (60 mg, 90.9% yield, TFA salt) as a gum.

LCMS Condition G: Rt=0.79 min. m/z 194.1 [M+H]⁺.

### Step-11—Preparation of 8-(4-chloro-2-fluorophenyl)-6-(2-(1-cyclopropyl-1H-pyrazol-4-yl)morpholino)-2,3-dimethylpyrimido[5,4-d]pyrimidin-4 (3H)-one

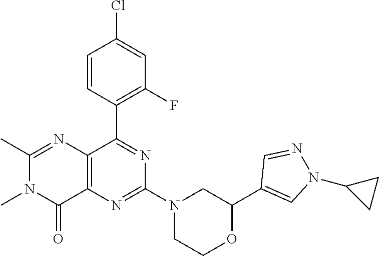 [SMILES: Cc1nc2c(-c3ccc(Cl)cc3F)nc(N3CCOC(c4cnn(C5CC5)c4)C3)nc2c(=O)n1C]

To a stirred solution of 2-(1-cyclopropyl-1H-pyrazol-4-yl)morpholine, TFA salt (60 mg, 0.31 mmol) and 6-chloro-8-(4-chloro-2-fluorophenyl)-2,3-dimethylpyrimido[5,4-d]pyrimidin-4 (3H)-one (105.08 mg, 0.31 mmol) in DMSO (3 mL) was added DIPEA (0.27 mL, 1.55 mmol). Resulting mixture was heated at 100° C. for 16 h. Reaction was purified by reverse phase preparative HPLC to afford 8-(4-chloro-2-fluorophenyl)-6-(2-(1-cyclopropyl-1H-pyrazol-4-yl)morpholino)-2,3-dimethylpyrimido[5,4-d]pyrimidin-4 (3H)-one (50 mg, 32.4% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then to 20% A and 80% B in 20 min., then to 5% A and 95% B in 21 min., held this composition up to 22 min. for column washing, then returned to initial composition in 23 min. and held till 25 min.

1H NMR (400 MHz, DMSO-d6) δ 7.83 (s, 1H), 7.66-7.59 (m, 2H), 7.45-7.44 (m, 2H), 4.61 (d, J=13.3 Hz, 1H), 4.50-4.47 (m, 2H), 4.00 (d, J=11.4 Hz, 1H), 3.69-3.67 (m, 2H), 3.50 (s, 3H), 3.18-3.13 (m, 2H), 2.44 (s, 3H), 1.02-1.00 (m, 2H), 0.94-0.92 (m, 2H).

LCMS Condition H: Rt=2.94 min. m/z 496.32 [M+H]⁺.

### Example 3

### 8-(4-chloro-2-fluorophenyl)-2,3-dimethyl-6-(2-(2-methylpyridin-4-yl)morpholino)pyrimido[5,4-d]pyrimidin-4 (3H)-one

### Step-1: Preparation of tert-butyl 2-(2-methylpyridin-4-yl)morpholine-4-carboxylate

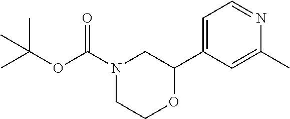 [SMILES: Cc1cc(C2CN(C(=O)OC(C)(C)C)CCO2)ccn1]

To a stirred solution of 4-(tert-butoxycarbonyl)morpholine-2-carboxylic acid (600 mg, 2.59 mmol) and 4-bromo-2-methylpyridine (444.15 mg, 2.59 mmol) in DMF (30 mL) were added cesium carbonate (2.5 g, 7.79 mmol) and 4,4′-Di-tert-butyl-2,2′-bipyridyl (104.42 mg, 0.39 mmol) and degassed with argon. Nickel(II) chloride ethylene glycol dimethyl ether complex (57.07 mg, 0.26 mmol) and 4,4-Bis(tert-butyl)-2,2-bipyridine]bis[3,5-difluoro-2-[5-(trifluoromethyl)-2-pyridinyl]phenyl]Iridium(III) hexafluorophosphate (29.12 mg, 0.03 mmol) were added to the reaction mass under inert atmosphere. Resulting mixture was irradiated with 450 nm LED light using an integrated photoreactor for 72 h. Reaction mixture was diluted with cold water and extracted with ethyl acetate. Combined organic layer was washed with water, brine, dried over anhydrous sodium sulphate, filtered and concentrated under reduced pressure. Crude mass was purified by combiflash chromatography (50-60% ethyl acetate in hexane) to afford tert-butyl 2-(2-methylpyridin-4-yl)morpholine-4-carboxylate (90 mg, 12.4% yield) as a gum.

1H NMR (400 MHz, DMSO) δ 8.39 (d, J=22.2 Hz, 1H), 7.25 (s, 1H), 7.17-7.11 (m, 1H), 4.38-4.23 (m, 1H), 3.96-3.56 (m, 4H), 2.50 (s, 3H), 2.46-2.39 (m. 2H), 1.42 (s, 9H).

LCMS Condition E: Rt=1.65 min. m/z 279.3 [M+H]⁺.

### Step-2: Preparation of 2-(2-methylpyridin-4-yl)morpholine

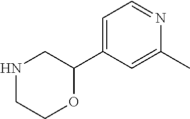

To a stirred solution of tert-butyl 2-(2-methylpyridin-4-yl)morpholine-4-carboxylate (90 mg, 0.32 mmol) in dioxane (2 mL) at 0° C. was added Dioxane-HCl (4M solution, 2 mL, 7.68 mmol) Resulting mixture was warmed to RT and stirred for 2 h. Reaction mixture was concentrated under reduced pressure and triturated with diethyl ether and pentane to afford 2-(2-methylpyridin-4-yl)morpholine (50 mg, 86.6% yield, HCl salt) as a gum.

LCMS Condition E: Rt=1.65 min. m/z 279.3 [M+H]⁺.

### Step-3: Preparation of 8-(4-chloro-2-fluorophenyl)-2,3-dimethyl-6-(2-(2-methylpyridin-4-yl)morpholino)pyrimido[5,4-d]pyrimidin-4 (3H)-one

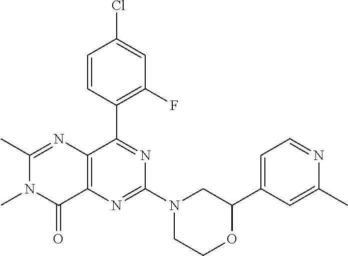

To a stirred solution of 6-chloro-8-(4-chloro-2-fluorophenyl)-2,3-dimethylpyrimido[5,4-d]pyrimidin-4 (3H)-one (80 mg, 0.24 mmol) and 2-(2-methylpyridin-4-yl)morpholine (46.34 mg, 0.26 mmol, HCl salt) in DMSO (2 mL) was added DIPEA (0.21 mL, 1.18 mmol). The resulting mixture was heated at 100° C. for 16 h. Reaction mixture purified by reverse phase prep HPLC to afford 8-(4-chloro-2-fluorophenyl)-2,3-dimethyl-6-(2-(2-methylpyridin-4-yl)morpholino)pyrimido[5,4-d]pyrimidin-4 (3H)-one (75 mg, 65.8% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 40% A and 60% B in 3 min, then to 30% A and 70% B in 20 min., then to 5% A and 95% B in 21 min., held this composition up to 22 min. for column washing, then returned to initial composition in 23 min. and held till 25 min.

1H NMR (400 MHz, DMSO) δ 8.44 (d, J=4.9 Hz, 1H), 7.68 (t, J=7.9 Hz, 1H), 7.61 (d, J=8.7 Hz, 1H), 7.46 (d, J=8.1 Hz, 1H), 7.30 (s, 1H), 7.23 (d, J=4.6 Hz, 1H), 4.70 (d, J=13.0 Hz, 1H), 4.68-4.57 (m, 2H), 4.15-4.09 (m, 1H), 3.76-3.71 (m, 1H), 3.51 (s, 3H), 3.23-3.18 (m, 1H), 3.00-2.94 (m, 1H), 2.49 (s, 3H), 2.44 (s, 3H).

LCMS Condition H: Rt=1.65 min. m/z 279.3 [M+H]⁺.

### Examples 4 to 10

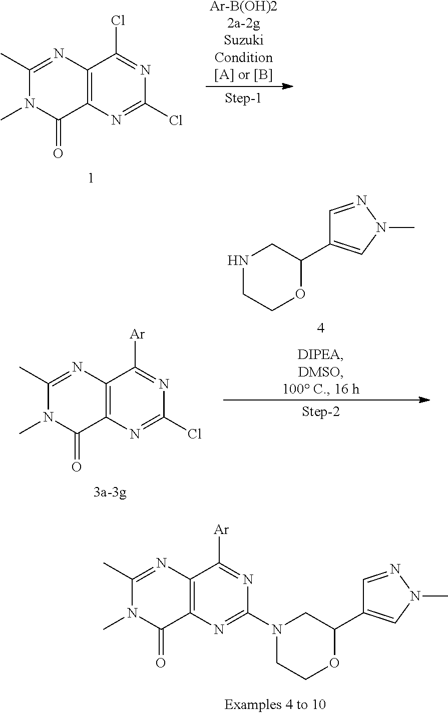

Condition-A: Na2CO3,PdCl2(dppf), Dioxane-H2O, 60° C., 1 h
Condition-B: Na2CO3,PdCl2(dppf), Dioxane-H2O, RT, 16 h

### Boronic Acids Used to Synthesize Examples 4 to 10:

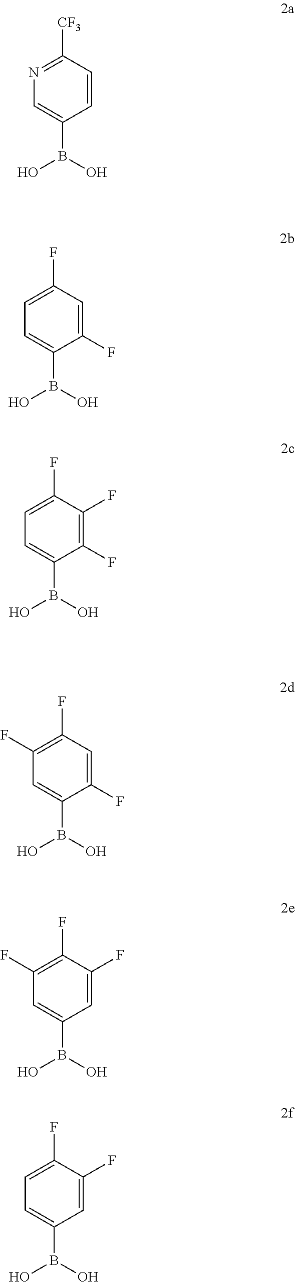 [SMILES: OB(O)c1ccc(C(F)(F)F)nc1 ; OB(O)c1ccc(F)cc1F ; OB(O)c1ccc(F)c(F)c1F ; OB(O)c1cc(F)c(F)cc1F ; OB(O)c1cc(F)c(F)c(F)c1 ; OB(O)c1ccc(F)c(F)c1]

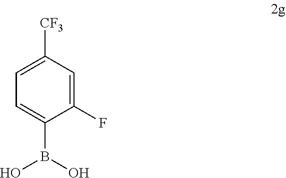 [SMILES: OB(O)c1ccc(C(F)(F)F)cc1F]

### General Procedure for Step-1

### Condition-A

To a stirred solution of 6,8-dichloro-2,3-dimethylpyrimido[5,4-d]pyrimidin-4 (3H)-one (1 mmol) and corresponding boronic acid (0.9 mmol) in dioxane (6 mL) and water (2 mL) was added sodium carbonate (1.5 mmol) and degassed with argon. PdCl2(dppf) (0.1 mmol) was added under inert atmosphere. The resulting mixture was heated at 60° C. for 1 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (20-40% ethyl acetate-hexane) to afford corresponding desired products 3a-3f.

### Condition-B

To a stirred solution of 6,8-dichloro-2,3-dimethylpyrimido[5,4-d]pyrimidin-4 (3H)-one (1 mmol) and corresponding boronic acid (0.9 mmol) in dioxane (6 mL) and water (2 mL) was added sodium carbonate (1.5 mmol) and degassed with argon. PdCl2(dppf) (0.1 mmol) was added under inert atmosphere. The resulting mixture was stirred at RT for 16 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (20-40% ethyl acetate-hexane) to afford corresponding desired products 3g.

Preparative HPLC methods (using Waters auto purification instrument):

Example 4: Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 45% A and 55% B in 3 min, then to 20% A and 80% B in 20 min., then to 5% A and 95% B in 21 min., held this composition up to 22 min. for column washing, then returned to initial composition in 23 min. and held till 25 min.

Example 5: Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 50% A and 50% B in 3 min, then to 30% A and 70% B in 20 min., then to 5% A and 95% B in 21 min., held this composition up to 22 min. for column washing, then returned to initial composition in 23 min. and held till 25 min.

Example 6: Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 45% A and 55% B in 3 min, then to 20% A and 80% B in 20 min., then to 5% A and 95% B in 21 min., held this composition up to 22 min. for column washing, then returned to initial composition in 23 min. and held till 25 min.

Example 7: Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 50% A and 50% B in 3 min, then to 25% A and 75% B in 20 min., then to 5% A and 95% B in 21 min., held this composition up to 22 min. for column washing, then returned to initial composition in 23 min. and held till 25 min.

Example 8: Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 50% A and 50% B, then to 40% A and 60% B in 20 min., then to 5% A and 95% B in 21 min., held this composition up to 22 min. for column washing, then returned to initial composition in 23 min. and held till 25 min.

Example 9: Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 50% A and 50% B, then to 20% A and 80% B in 20 min., then to 5% A and 95% B in 21 min., held this composition up to 22 min. for column washing, then returned to initial composition in 23 min. and held till 25 min.

Example 10: Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 50% A and 50% B in 3 min, then to 30% A and 70% B in 20 min., then to 5% A and 95% B in 21 min., held this composition up to 22 min. for column washing, then returned to initial composition in 23 min. and held till 25 min.

### General Procedure for Step-2

To a stirred solution of 2-(1-methyl-1H-pyrazol-4-yl)morpholine, HCl salt (0.59 mmol) in DMSO (2 mL) was added DIPEA (1.18 mmol) at RT and stirred for 15 min. To it was added corresponding compounds 3a to 3g (0.29 mmol) at RT.

Resulting mixture was heated at 100° C. for 16 h. Reaction mixture was concentrated under reduced pressure. Crude mass was purified by reverse phase preparative HPLC to afford corresponding target compounds.

HNMR, LCMS and yield percentage information of compounds 3a to 3g and examples 4 to 10 are provided in the table below:

| | | | | | |
|---|---|---|---|---|---|
| Compound | | | | | Yield |
| No. | Structure | Name | HNMR | LCMS | (%) |

| | | | | |
|---|---|---|---|---|
| 3a 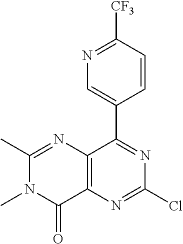 [SMILES: Cc1nc2c(-c3ccc(C(F)(F)F)nc3)nc(Cl)nc2c(=O)n1C] | 6-chloro-2,3- dimethyl-8-(6- (trifluoromethyl)py- ridin-3- yl)pyrimido[5,4- d]pyrimidin-4(3H)- one | 1H NMR (400 MHz, DMSO-d6) δ 9.60 (s, 1 H), 8.90 (d, J = 7.88 Hz, 1 H), 8.16 (d, J = 8.04 Hz, 1 H), 3.60 (s, 3 H), 2.66 (s, 3 H). | LCMS Condition G: Rt = 2.04 min. m/z 356.2 [M + H]⁺. | 68.8 |
| Ex. 4 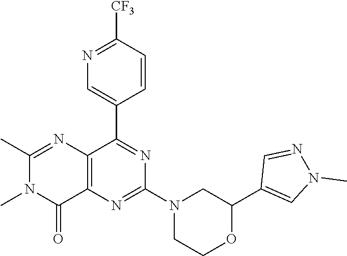 | 2,3-dimethyl-6-(2- (1-methyl-1H- pyrazol-4- yl)morpholino)-8- (6- (trifluoromethyl)py- ridin-3- yl)pyrimido[5,4- d]pyrimidin-4(3H)- one | 1HNMR (400 MHz, DMSO-d6) δ 9.59 (s, 1 H), 8.90-8.89 (m, 1 H), 8.10 (d, J = 8.2 Hz, 1 H), 7.77 (s, 1 H), 7.48 (s, 1 H), 4.71 (d, J = 12.6 Hz, 1 H), 4.60 (d, J = 13.2 Hz, 1 H), 4.54-4.51 (m, 1 H), 4.04-4.01 (m, 1 H), 3.82 (s, 3 H), 3.71-3.64 (m, 1 H), 3.54 (s, 3H), 3.26- 3.15 (m, 2 H), 2.54 (s, 3H). | LCMS Condition H: Rt = 2.74 min. m/z 487.32 [M + H]⁺. | 58.8 |
| 3b 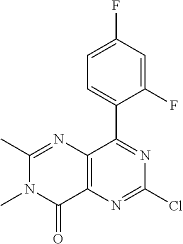 [SMILES: Cc1nc2c(-c3ccc(F)cc3F)nc(Cl)nc2c(=O)n1C] | 6-chloro-8-(2,4- difluorophenyl)- 2,3- dimethylpyrimido[5,4- d]pyrimidin- 4(3H)-one | 1H NMR (400 MHz, DMSO-d6) δ 7.79-7.73 (m, 1 H), 7.51-7.46 (m, 1 H), 7.33-7.29 (m, 1 H), 3.57 (s, 3 H), 2.55 (s, 3 H). | LCMS Condition F: Rt = 1.97 min. m/z 323.2 [M + H]⁺. | 75.6 |
| Ex. 5 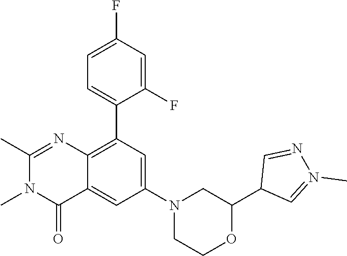 | 8-(2,4- difluorophenyl)- 2,3-dimethyl-6-(2- (1-methyl-1H- pyrazol-4- yl)morpholino)pyri- mido[5,4- d]pyrimidin-4(3H)- one | 1HNMR (400 MHz, DMSO-d6) δ 7.74-7.67 (m, 2 H), 7.45-7.37 (m, 2 H), 7.25-7.24 (m, 1 H), 4.62 (d, J = 13.0 Hz, 1 H), 4.51 (d, J = 10.9 Hz, 2 H), 4.00 (d, J = 11.0 Hz, 1 H), 3.81 (s, 3 H), 3.66 (t, J = 10.0 Hz, 1 H), 3.51 (s, 3 H), 3.21-3.12 (m, 2 H), 2.44 (s, 3 H). | LCMS Condition H: Rt = 2.72 min. m/z 454.27 [M + H]⁺. | 51.6 |
| 3c 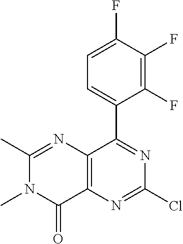 [SMILES: Cc1nc2c(-c3ccc(F)c(F)c3F)nc(Cl)nc2c(=O)n1C] | 6-chloro-2,3- dimethyl-8-(2,3,4- trifluorophenyl)pyri- mido[5,4- d]pyrimidin-4(3H)- one | 1H NMR (400 MHz, DMSO-d6) δ 7.58-7.54 (m, 2 H), 3.57 (s, 3 H), 2.56 (s, 3 H). | LCMS Condition E: Rt = 1.81 min. m/z 341.19 [M + H]⁺. | 71.6 |
| Ex. 6 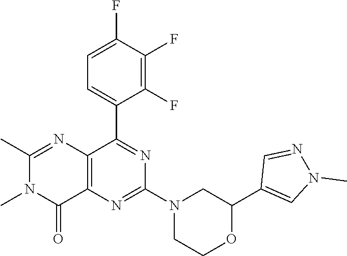 | 2,3-dimethyl-6-(2- (1-methyl-1H- pyrazol-4- yl)morpholino)-8- (2,3,4- trifluorophenyl)pyri- mido[5,4- d]pyrimidin-4(3H)- one | 1H NMR (400 MHz, DMSO-d6) δ 7.74 (s, 1 H), 7.55-7.49 (m, 2 H), 7.45 (s, 1 H), 4.62 (d, J = 12.9 Hz, 1 H), 4.51 (d, J = 10.6 Hz, 2 H), 4.00 (d, J = 10.9 Hz, 1 H), 3.81 (s, 3 H), 3.72-3.61 (m, 1 H), 3.51 (s, 3 H), 3.22-3.13 (m, 2 H), 2.45 (s, 3 H). | LCMS Condition G: Rt = 3.11 min. m/z 472.3 [M + H]⁺. | 57.7 |
| 3d 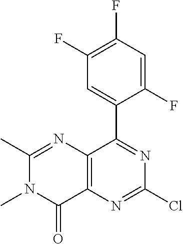 [SMILES: Cc1nc2c(-c3cc(F)c(F)cc3F)nc(Cl)nc2c(=O)n1C] | 6-chloro-2,3- dimethyl-8-(2,4,5- trifluorophenyl)pyri- mido[5,4- d]pyrimidin-4(3H)- one | 1H NMR (400 MHz, DMSO-d6) δ 7.87-7.77 (m, 2 H), 3.57 (s, 3 H), 2.56 (s, 3 H). | LCMS Condition E: Rt = 1.81 min. m/z 341.3 [M + H]⁺. | 71.9 |
| Ex. 7 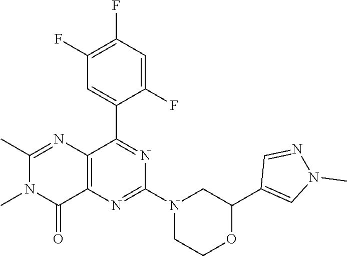 | 2,3-dimethyl-6-(2- (1-methyl-1H- pyrazol-4- yl)morpholino)-8- (2,4,5- trifluorophenyl)pyri- mido[5,4- d]pyrimidin-4(3H)- one | 1H NMR (400 MHz, DMSO-d6) δ 7.80-7.75 (m, 3 H), 7.45 (s, 1 H), 4.72-4.56 (m, 1 H), 4.51- 4.49 (m, 2 H), 4.01-3.98 (m, 1 H), 3.81 (s, 3 H), 3.65 (t, J = 10.1 Hz, 1 H), 3.50 (s, 3 H), 3.18 (q, J = 13.6 Hz, 2 H), 2.44 (s, 3 H). | LCMS Condition G: Rt = 2.78 min. m/z 472.3 [M + H]⁺. | 41.1 |
| 3e 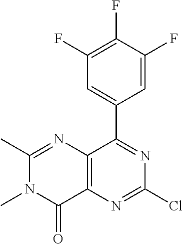 [SMILES: Cc1nc2c(-c3cc(F)c(F)c(F)c3)nc(Cl)nc2c(=O)n1C] | 6-chloro-2,3- dimethyl-8-(3,4,5- trifluorophenyl)pyri- mido[5,4- d]pyrimidin-4(3H)- one | 1H NMR (400 MHz, DMSO-d6) δ 8.39-8.35 (m, 2 H), 3.58 (s, 3 H), 2.66 (s, 3 H). | LCMS Condition F: Rt = 2.19 min. m/z 341.2 [M + H]⁺. | 71.6 |
| Ex. 8 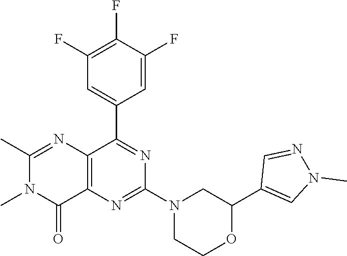 | 2,3-dimethyl-6-(2- (1-methyl-1H- pyrazol-4- yl)morpholino)-8- (3,4,5- trifluorophenyl)pyri- mido[5,4- d]pyrimidin-4(3H)- one | 1H NMR (400 MHz, DMSO-d6) δ 8.38 (t, J = 7.7 Hz, 2 H), 7.76 (s, 1 H), 7.47 (s, 1 H), 4.68- 4.52 (m, 2 H), 4.51 (dd, J = 2.7, 10.5 Hz, 1 H), 4.03- 4.00 (m, 1 H), 3.82 (s, 3 H), 3.69-3.64 (m, 1 H), 3.52 (s, 3 H), 3.26-3.10 (m, 2 H), 2.54 (s, 3 H). | LCMS Condition H: Rt = 2.94 min. m/z 472.27 [M + H]⁺. | 57.7 |
| 3f 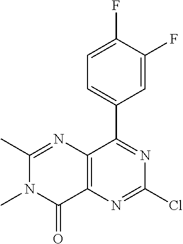 [SMILES: Cc1nc2c(-c3ccc(F)c(F)c3)nc(Cl)nc2c(=O)n1C] | 6-chloro-8-(3,4- difluorophenyl)- 2,3- dimethylpyrimido[5,4- d]pyrimidin- 4(3H)-one | 1H NMR (400 MHz, DMSO-d6) δ 8.51-8.46 (m, 1 H), 8.36-8.32 (m, 1 H), 7.70-7.63 (m, 1 H), 3.58 (s, 3 H), 2.65 (s, 3 H). | LCMS Condition F: Rt = 2.1 min. m/z 323.2 [M + H]⁺. | 75.6 |
| Ex. 9 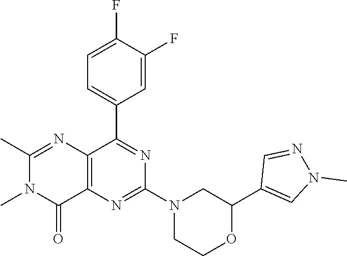 | 8-(3,4- difluorophenyl)- 2,3-dimethyl-6-(2- (1-methyl-1H- pyrazol-4- yl)morpholino)pyri- mido[5,4- d]pyrimidin-4(3H)- one | 1H NMR (400 MHz, DMSO-d6) δ 8.48-8.44 (m, 1 H), 8.36-8.20 (m, 1 H), 7.76 (s, 1 H), 7.60 (q, J = 8.6 Hz, 1 H), 7.47 (s, 1 H), 4.66 (d, J = 12.7 Hz, 1 H), 4.59-4.49 (m, 2 H), 4.02 (d, J = 10.4 Hz, 1 H), 3.83 (s, 3 H), 3.66 (t, J = 11.0 Hz, 1 H), 3.51 (s, 3 H), 3.26-3.07 (m, 2 H), 2.53 (s, 3 H). | LCMS Condition H: Rt = 2.84 min. m/z 454.27 [M + H]⁺. | 56.8 |
| 3g 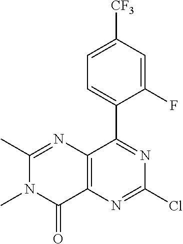 [SMILES: Cc1nc2c(-c3ccc(C(F)(F)F)cc3F)nc(Cl)nc2c(=O)n1C] | 6-chloro-8-(2- fluoro-4- (trifluoromethyl)phe- nyl)-2,3- dimethylpyrimido[5,4- d]pyrimidin- 4(3H)-one | 1H NMR (400 MHz, DMSO-d6) δ 7.96-7.89 (m, 2 H), 7.81 (d, J = 7.44 Hz, 1 H), 3.57 (s, 3 H), 2.54 (s, 3 H). | LCMS Condition F: Rt = 2.15 min. m/z 373.2 [M + H]⁺. | 65.5 |
| Ex. 10 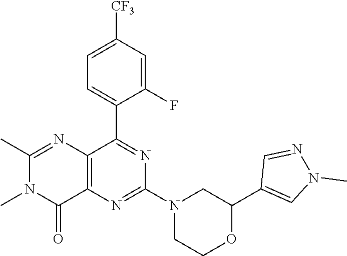 | 8-(2-fluoro-4- (trifluoromethyl)phe- nyl)-2,3- dimethyl-6-(2-(1- methyl-1H- pyrazol-4- yl)morpholino)pyri- mido[5,4- d]pyrimidin-4(3H)- one | 1H NMR (400 MHz, DMSO-d6) δ 7.88-7.84 (m, 2 H), 7.75 (d, J = 7.0 Hz, 2 H), 7.45 (s, 1 H), 4.62 (d, J = 13.0 Hz, 1 H), 4.56-4.46 (m, 2 H), 4.01 (d, J = 11.1 Hz, 1 H), 3.81 (s, 3 H), 3.69-3.64 (m, 1 H), 3.52 (s, 3 H), 3.23- 3.13 (m, 2 H), 2.43 (s, 3 H). | LCMS Condition H: Rt = 2.99 min. m/z 504.29 [M + H]⁺. | 66.5 |

### Example 11

### 8-(4,4-difluorocyclohexyl)-2,3-dimethyl-6-(2-(1-methyl-1H-pyrazol-4-yl)morpholino)pyrimido[5,4-d]pyrimidin-4 (3H)-one

### Step-1—Preparation of 6-chloro-2,3-dimethyl-8-(1,4-dioxaspiro[4.5]dec-7-en-8-yl)pyrimido[5,4-d]pyrimidin-4 (3H)-one

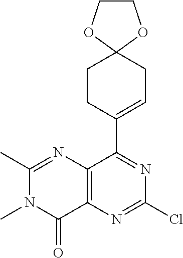 [SMILES: Cc1nc2c(C3=CCC4(CC3)OCCO4)nc(Cl)nc2c(=O)n1C]

To a stirred solution of 6,8-dichloro-2,3-dimethylpyrimido[5,4-d]pyrimidin-4 (3H)-one (300 mg, 1.23 mmol) and 4,4,5,5-tetramethyl-2-(1,4-dioxaspiro[4.5]dec-7-en-8-yl)-1,3,2-dioxaborolane (294.4 mg, 1.107 mmol) in dioxane (4 mL) and water (1 mL) was added sodium carbonate (258.3 mg, 2.46 mmol) and the resulting solution was degassed with argon. PdCl2(dppf) (90.0 mg, 0.123 mmol) was added under inert atmosphere. The resulting mixture was heated at 80° C. for 2 h. The reaction mixture was diluted with ethyl acetate, filtered through a short pad of celite and washed with ethyl acetate. The combined organic layers were washed with water, brine, dried over anhydrous sodium sulphate, filtered and concentrated under reduced pressure. The crude product was purified by column chromatography (50% ethyl acetate-hexane) to afford 2,3-dimethyl-6-(2-(1-methyl-1H-pyrazol-4-yl)morpholino)-8-(1,4-dioxaspiro[4.5]dec-7-en-8-yl)pyrimido[5,4-d]pyrimidin-4 (3H)-one (190 mg, 41.9% yield) as off-white solid.

1H NMR (400 MHz, DMSO-d6) δ 7.56 (t, J=4.0 Hz, 1H), 3.94 (s, 4H), 3.54 (s, 3H), 2.81-2.72 (m, 2H), 2.60 (s, 3H), 2.59-2.53 (m, 2H), 1.82 (t, J=6.4 Hz, 2H).

LCMS Condition N: Rt=1.82 min. m/z 349.3 [M+H]⁺.

### Step-2—Preparation of 2,3-dimethyl-6-(2-(1-methyl-1H-pyrazol-4-yl)morpholino)-8-(1,4-dioxaspiro[4.5]dec-7-en-8-yl)pyrimido[5,4-d]pyrimidin-4 (3H)-one

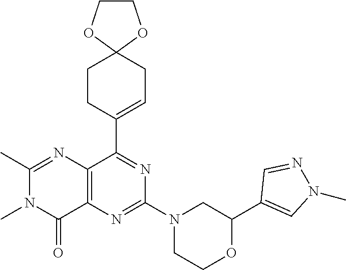

Procedure of Step-5, example-1 was followed using 2,3-dimethyl-6-(2-(1-methyl-1H-pyrazol-4-yl)morpholino)-8-(1,4-dioxaspiro[4.5]dec-7-en-8-yl)pyrimido[5,4-d]pyrimidin-4 (3H)-one and 2-(1-methyl-1H-pyrazol-4-yl)morpholine hydrochloride salt as starting materials to afford 2,3-dimethyl-6-(2-(1-methyl-1H-pyrazol-4-yl)morpholino)-8-(1,4-dioxaspiro[4.5]dec-7-en-8-yl)pyrimido[5,4-d]pyrimidin-4 (3H)-one as sticky liquid in 42.1% yield.

1H NMR (400 MHz, DMSO-d6) δ 7.75 (s, 1H), 7.46 (s, 1H), 7.39 (br s, 1H), 4.60 (d, J=13.4 Hz, 1H), 4.49 (t, J=9.7 Hz, 2H), 4.03-3.95 (m, 1H), 3.92 (s, 4H), 3.82 (s, 3H), 3.63 (t, J=11.0 Hz, 1H), 3.49 (s, 3H), 3.13 (q, J=14.2 Hz, 2H), 2.77-2.72 (m, 2H), 2.54-2.50 (m, 5H), 1.80 (t, J=6.5 Hz, 2H).

LCMS Condition N: Rt=1.79 min. m/z 480.4 [M+H]⁺.

### Step-3—Preparation of 2,3-dimethyl-6-(2-(1-methyl-1H-pyrazol-4-yl)morpholino)-8-(1,4-dioxaspiro[4.5]decan-8-yl)pyrimido[5,4-d]pyrimidin-4 (3H)-one

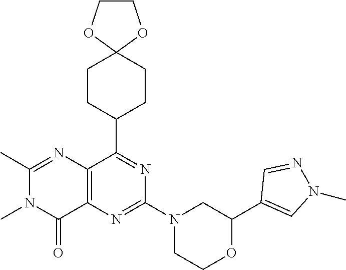

To a degassed solution of 2,3-dimethyl-6-(2-(1-methyl-1H-pyrazol-4-yl)morpholino)-8-(1,4-dioxaspiro[4.5]dec-7-en-8-yl)pyrimido[5,4-d]pyrimidin-4 (3H)-one (110 mg, 0.23 mmol) in ethanol (10 mL), was added 10% Pd—C (30 mg). The resulting mixture was hydrogenated at RT under balloon pressure for 5 h. Reaction mixture was filtered through a short pad of celite and washed with ethanol. Combined filtrate was concentrated under reduced pressure to afford 2,3-dimethyl-6-(2-(1-methyl-1H-pyrazol-4-yl)morpholino)-8-(1,4-dioxaspiro[4.5]decan-8-yl)pyrimido[5,4-d]pyrimidin-4 (3H)-one (100 mg, 90.4% yield) as a gum.

1H NMR (400 MHz, DMSO-d6) δ 7.73 (s, 1H), 7.43 (s, 1H), 4.61-4.43 (m, 3H), 4.05-3.92 (m, 1H), 3.85 (s, 4H), 3.80 (s, 3H), 3.70-3.55 (m, 2H), 3.47 (s, 3H), 3.18-3.05 (m, 1H), 2.55-2.50 (m, 6H), 1.85-1.53 (m, 6H).

LCMS Condition O: Rt=2.81 min. m/z 482.1 [M+H]⁺.

### Step-4—Preparation of 2,3-dimethyl-6-(2-(1-methyl-1H-pyrazol-4-yl)morpholino)-8-(4-oxocyclohexyl)pyrimido[5,4-d]pyrimidin-4 (3H)-one

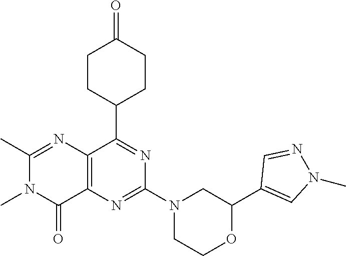

To a stirred solution of 2,3-dimethyl-6-(2-(1-methyl-1H-pyrazol-4-yl)morpholino)-8-(1,4-dioxaspiro[4.5]decan-8-yl)pyrimido[5,4-d]pyrimidin-4 (3H)-one (100 mg, 0.208 mmol) in THF (5 mL) was added 2(N) HCl (1.0 mL). The resulting mixture was heated at 70° C. for 3 h. The reaction mixture was quenched with water, extracted with ethyl acetate and concentrated under reduced pressure to afford 2,3-dimethyl-6-(2-(1-methyl-1H-pyrazol-4-yl)morpholino)-8-(4-oxocyclohexyl)pyrimido[5,4-d]pyrimidin-4 (3H)-one (85 mg, crude) as brown liquid. The crude material was used in the next step without further purification.

1H NMR (400 MHz, DMSO-d6) δ 7.74 (s, 1H), 7.45 (s, 1H), 4.67-4.38 (m, 3H), 4.20-3.90 (m, 3H), 3.81 (s, 3H), 3.65-3.60 (m, 1H), 3.51 (s, 3H), 2.50 (s, 3H), 2.37-2.22 (m, 3H), 2.18-1.90 (m, 6H).

LCMS Condition P: Rt=1.63 min. m/z 438.48 [M+H]⁺.

### Step-5—Preparation of 8-(4,4-difluorocyclohexyl)-2,3-dimethyl-6-(2-(1-methyl-1H-pyrazol-4-yl)morpholino)pyrimido[5,4-d]pyrimidin-4 (3H)-one (Example-11)

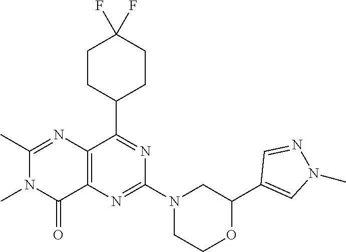

To a stirred solution of 2,3-dimethyl-6-(2-(1-methyl-1H-pyrazol-4-yl)morpholino)-8-(4-oxocyclohexyl)pyrimido[5,4-d]pyrimidin-4 (3H)-one (85.0 mg, 0.195 mmol) in DCM (5.0 mL) at 0° C. was added DAST (0.261 mL, 1.945 mmol). The resulting mixture was stirred at RT for 16 h. The reaction was quenched with water, extracted with ethyl acetate and concentrated under reduced pressure. The crude material was purified by reverse phase HPLC to afford the pure product (7.0 mg, 8.2% yield) as off white solid. Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 60% A and 40% B in 3 min, then to 35% A and 65% B in 20 min., then to 5% A and 95% B in 21 min., held this composition up to 22 min. for column washing, then returned to initial composition in 23 min. and held till 25 min.

1H NMR (400 MHz, DMSO) δ 7.75 (s, 1H), 7.45 (s, 1H), 4.61 (d, J=13.1 Hz, 1H), 4.49 (t, J=10.2 Hz, 2H), 3.99 (d, J=10.9 Hz, 1H), 3.82 (s, 3H), 3.76 (m, 1H), 3.63-3.61 (m, 1H), 3.49 (s, 3H), 3.14-3.09 (m, 2H), 2.54 (s, 3H), 2.12-2.06 (m, 3H), 2.05-1.79 (m, 5H).

LCMS Condition M: Rt=2.77 min. m/z 460.45 [M+H]⁺.

### Example 12

### Example-12A: Diastereomer 1

### 2,3-dimethyl-6-(2-(1-methyl-1H-pyrazol-4-yl)morpholino)-8-((1s,4s)-4-methylcyclohexyl)pyrimido[5,4-d]pyrimidin-4 (3H)-one

### Example-12B: Diastereomer 2

### 2,3-dimethyl-6-(2-(1-methyl-1H-pyrazol-4-yl)morpholino)-8-((1r,4r)-4-methylcyclohexyl)pyrimido[5,4-d]pyrimidin-4 (3H)-one

### Step-1—Preparation of 6-chloro-2,3-dimethyl-8-(4-methylcyclohex-1-en-1-yl)pyrimido[5,4-d]pyrimidin-4 (3H)-one

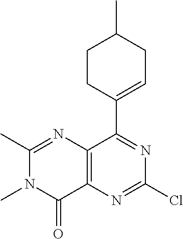 [SMILES: Cc1nc2c(C3=CCC(C)CC3)nc(Cl)nc2c(=O)n1C]

To a stirred solution of 6-chloro-2,3-dimethyl-8-(4-methylcyclohex-1-en-1-yl)pyrimido[5,4-d]pyrimidin-4 (3H)-one (300 mg, 1.22 mmol) and 4,4,5,5-tetramethyl-2-(4-methylcyclohex-1-en-1-yl)-1,3,2-dioxaborolane (244.6 mg, 1.10 mmol) in dioxane (6 mL) and water (1.5 mL) was added cesium carbonate (598.4 mg, 1.84 mmol) and degassed with argon. PdCl2(dppf)·DCM (99.91 mg, 0.12 mmol) was added under inert atmosphere. The resulting mixture was heated at 80° C. for 1 h. The reaction mixture was diluted with ethyl acetate, filtered through a short pad of celite and washed with ethyl acetate. The combined organic layers were washed with water, brine, dried over anhydrous sodium sulphate, filtered and concentrated under reduced pressure. The crude product was purified by column chromatography (50% ethyl acetate-hexane) to afford 6-chloro-2,3-dimethyl-8-(4-methylcyclohex-1-en-1-yl)pyrimido[5,4-d]pyrimidin-4 (3H)-one (126 mg, 34.2% yield) as off-white solid.

1H NMR (400 MHz, DMSO-d6) δ 7.63 (s, 1H), 3.53 (s, 3H), 2.60 (s, 3H), 2.11-1.60 (m, 4H), 1.45-1.14 (m, 3H), 1.01 (d, J=6.4 Hz, 3H).

LCMS Condition N: Rt=2.25 min. m/z 305.3 [M+H]⁺.

### Step-2—Preparation of 2,3-dimethyl-6-(2-(1-methyl-1H-pyrazol-4-yl)morpholino)-8-(4-methylcyclohex-1-en-1-yl)pyrimido[5,4-d]pyrimidin-4 (3H)-one

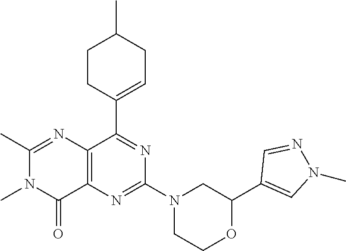

Procedure of Step-5, example-1 was followed using 6-chloro-2,3-dimethyl-8-(4-methylcyclohex-1-en-1-yl)pyrimido[5,4-d]pyrimidin-4 (3H)-one and 2-(1-methyl-1H-pyrazol-4-yl)morpholine hydrochloride salt as starting materials to afford 2,3-dimethyl-6-(2-(1-methyl-1H-pyrazol-4-yl)morpholino)-8-(4-methylcyclohex-1-en-1-yl)pyrimido[5,4-d]pyrimidin-4 (3H)-one as yellow semi-solid in (81.6% yield).

1H NMR (400 MHz, DMSO-d6) δ 7.74 (s, 1H), 7.48-7.39 (m, 2H), 4.60 (d, J=13.6 Hz, 1H), 4.49 (t, J=10.1 Hz, 2H), 3.99 (d, J=11.1 Hz, 1H), 3.82 (s, 3H), 3.63 (t, J=11.0 Hz, 1H), 3.49 (s, 3H), 3.19-3.04 (m, 2H), 2.75-2.37 (m, 6H), 2.09-1.61 (m, 3H), 1.46-1.14 (m, 1H), 0.99 (d, J=6.4 Hz, 3H).

LCMS Condition N: Rt=2.16 min. m/z 436.5 [M+H]⁺.

### Step-3—Preparation of 2,3-dimethyl-6-(2-(1-methyl-1H-pyrazol-4-yl)morpholino)-8-((1s,4s)-4-methylcyclohexyl)pyrimido[5,4-d]pyrimidin-4 (3H)-one (Example-12A: Diastereomer 1) and 2,3-dimethyl-6-(2-(1-methyl-1H-pyrazol-4-yl)morpholino)-8-((1r,4r)-4-methylcyclohexyl)pyrimido[5,4-d]pyrimidin-4 (3H)-one (Example-12B: Diastereomer 2)

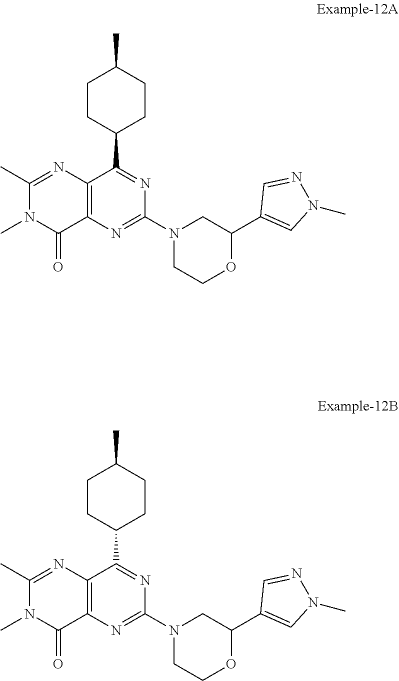

Procedure of Step-3, example-11 was followed using 2,3-dimethyl-6-(2-(1-methyl-1H-pyrazol-4-yl)morpholino)-8-(4-methylcyclohex-1-en-1-yl)pyrimido[5,4-d]pyrimidin-4 (3H)-one as starting material followed by diastereomer separation by reverse phase prep HPLC to afford 2,3-dimethyl-6-(2-(1-methyl-1H-pyrazol-4-yl)morpholino)-8-((1s,4s)-4-methylcyclohexyl)pyrimido[5,4-d]pyrimidin-4 (3H)-one as off white solid in (14.1%) yield and 2,3-dimethyl-6-(2-(1-methyl-1H-pyrazol-4-yl)morpholino)-8-((1r,4r)-4-methylcyclohexyl)pyrimido[5,4-d]pyrimidin-4 (3H)-one as off white solid in (9.3%) yield. Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

Example-12A: 1H NMR (400 MHz, DMSO-d6) δ 7.73 (s, 1H), 7.44 (s, 1H), 4.62 (d, J=11.8 Hz, 1H), 4.56-4.46 (m, 2H), 4.00 (d, J=10.0 Hz, 1H), 3.82 (s, 3H), 3.77-3.57 (m, 2H), 3.50 (s, 3H), 3.23-3.08 (m, 2H), 2.53 (s, 3H), 2.00-1.70 (m, 3H), 1.73-1.42 (m, 6H), 0.97 (d, J=6.8 Hz, 3H).

LCMS Condition M: Rt=3.03 min. m/z 438.39 [M+H]⁺.

Example-12B: 1H NMR (400 MHz, DMSO) δ 7.75 (s, 1H), 7.45 (s, 1H), 4.61 (d, J=12.9 Hz, 1H), 4.49 (t, J=10.6 Hz, 2H), 3.99 (d, J=10.9 Hz, 1H), 3.82 (s, 3H), 3.68-3.53 (m, 2H), 3.49 (s, 3H), 3.12 (q, J=11.9 Hz, 2H), 2.53 (s, 3H), 1.91-1.70 (m, 4H), 1.59 (q, J=11.8 Hz, 2H), 1.49-1.34 (m, 1H), 1.09 (q, J=12.9 Hz, 2H), 0.92 (d, J=6.2 Hz, 3H).

LCMS Condition M: Rt=3.05 min. m/z 438.39 [M+H]⁺.

### Example 13

### 8-(4-chloro-2-fluorophenyl)-6-(4,4-difluoro-3-(1-methyl-1H-pyrazol-4-yl)piperidin-1-yl)-2,3-dimethylpyrimido[5,4-d]pyrimidin-4 (3H)-one

### Step-1—Preparation of 4-methoxy-3-(1-methyl-1H-pyrazol-4-yl)pyridine

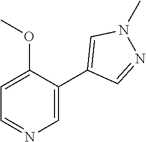

To a stirred solution of 3-bromo-4-methoxypyridine (3 g, 15.96 mmol) and 1-methyl-4-(4,4,5,5-tetramethyl-1,3,2-dioxaborolan-2-yl)-1H-pyrazole (4.98 g, 23.94 mmol) in dioxane (40 mL) and water (24 mL) was added sodium carbonate (5.07 g, 47.87 mmol) and degassed with argon. Pd(pph3)4 (922 mg, 0.798 mmol) was added under inert atmosphere. The resulting mixture was heated at 120° C. for 4 h. The reaction mixture was diluted with ethyl acetate, filtered through a short pad of celite and washed with ethyl acetate. The combined organic layers were washed with water, brine, dried over anhydrous sodium sulphate, filtered and concentrated under reduced pressure. The crude product was purified by column chromatography (2-5% methanol-dichloromethane) to afford 4-methoxy-3-(1-methyl-1H-pyrazol-4-yl)pyridine (2.8 g, 92.2% yield) as a gum.

1H NMR (400 MHz, DMSO) δ 8.70 (s, 1H), 8.30 (d, J=5.6 Hz, 1H), 8.17 (s, 1H), 7.96 (s, 1H), 7.09 (d, J=5.6 Hz, 1H), 3.93 (s, 3H), 3.88 (s, 3H).

LCMS Condition N: Rt=0.29 min. m/z 190.2 [M+H]⁺.

### Step-2—Preparation of benzyl 5-(1-methyl-1H-pyrazol-4-yl)-4-oxo-3,4-dihydropyridine-1 (2H)-carboxylate

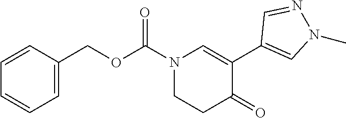 [SMILES: Cn1cc(C2=CN(C(=O)OCc3ccccc3)CCC2=O)cn1]

To a stirred solution of 4-methoxy-3-(1-methyl-1H-pyrazol-4-yl)pyridine (4 g, 21.16 mmol) in methanol (40 mL) was added sodium borohydride (1.6 g, 42.33 mmol) portion wise at −78° C. and stirred for 2 h maintaining temperature at −78° C. A solution of benzyl chloroformate (3.61 ml, 25.38 mmol) in dry ethyl ether (3 ml) was then added dropwise to the reaction mixture at −78° C. Resulting solution was warmed to RT and stirred for 3 h. The reaction mixture was quenched with water under cooling condition and extracted with ethyl acetate. The combined organic layers were washed with water, brine, dried over anhydrous sodium sulphate, filtered and concentrated under reduced pressure. The crude product was purified by column chromatography (50% EA in hexane) to afford benzyl 5-(1-methyl-1H-pyrazol-4-yl)-4-oxo-3,4-dihydropyridine-1 (2H)-carboxylate (1.2 g, 19.3% yield) as a gum.

1H NMR (400 MHz, DMSO-d6) δ 8.07 (s, 1H), 7.95 (s, 1H), 7.63 (s, 1H), 7.50-7.34 (m, 5H), 5.28 (s, 2H), 4.01 (t, J=7.6 Hz, 2H), 3.80 (s, 3H), 2.62 (t, J=7.2 Hz, 2H).

LCMS Condition N: Rt=1.83 min. m/z 312.3 [M+H]⁺.

### Step-3—Preparation of 5-(1-methyl-1H-pyrazol-4-yl)-2,3-dihydropyridin-4 (1H)-one

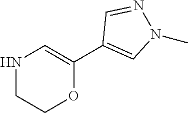

Procedure of Step-3, example-11 was followed using benzyl 5-(1-methyl-1H-pyrazol-4-yl)-4-oxo-3,4-dihydropyridine-1 (2H)-carboxylate as starting material to afford 5-(1-methyl-1H-pyrazol-4-yl)-2,3-dihydropyridin-4 (1H)-one as brown semi-solid in 95.7% yield.

LCMS Condition N: Rt=0.39 min. m/z 178.2 [M+H]⁺.

### Step-4—Preparation of tert-butyl 5-(1-methyl-1H-pyrazol-4-yl)-4-oxo-3,4-dihydropyridine-1 (2H)-carboxylate

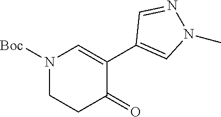 [SMILES: Cn1cc(C2=CN(C(=O)OC(C)(C)C)CCC2=O)cn1]

To a stirred solution of 5-(1-methyl-1H-pyrazol-4-yl)-2,3-dihydropyridin-4 (1H)-one (600 mg, 3.39 mmol) in DCM (20 ml) was added triethyl amine (1.53 ml, 10.17 mmol) followed by Boc-anhydride (1108.4 mg, 5.08 mmol) at 10° C. Resulting mixture was stirred at RT for 16 h. The reaction mixture was diluted with cold water and extracted with DCM. The combined organic layers were washed with water, brine, dried over anhydrous sodium sulphate, filtered and concentrated under reduced pressure. The crude product was purified by column chromatography (60% EA in Hexane) to afford tert-butyl 5-(1-methyl-1H-pyrazol-4-yl)-4-oxo-3,4-dihydropyridine-1 (2H)-carboxylate (600 mg, 63.2% yield) as a gum.

1H NMR (400 MHz, DMSO-d6) δ 8.18 (br s, 1H), 7.94 (s, 1H), 7.62 (s, 1H), 3.98-3.89 (m, 2H), 3.80 (s, 3H), 2.62-2.56 (m, 2H), 1.51 (s, 9H).

LCMS Condition N: Rt=1.81 min. m/z 278.3 [M+H]⁺.

### Step-5—Preparation of tert-butyl 3-(1-methyl-1H-pyrazol-4-yl)-4-oxopiperidine-1-carboxylate

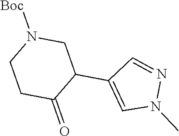 [SMILES: Cn1cc(C2CN(C(=O)OC(C)(C)C)CCC2=O)cn1]

To a degassed solution of tert-butyl 5-(1-methyl-1H-pyrazol-4-yl)-4-oxo-3,4-dihydropyridine-1 (2H)-carboxylate (200 mg, 0.72 mmol) in ethyl acetate (10 mL), Pd(OH)₂ (51.35 mg, 0.36 mmol) was added. The resulting mixture was stirred at RT under H2 gas balloon pressure for 16 h. The reaction mixture was filtered through a short pad of celite and washed with ethanol. The combined filtrate was concentrated under reduced pressure. The crude material was purified by column chromatography to afford tert-butyl 3-(1-methyl-1H-pyrazol-4-yl)-4-oxopiperidine-1-carboxylate (110 mg, 55.4% yield) as a gum.

1H NMR (400 MHz, DMSO-d6) δ 7.53 (s, 1H), 7.27 (s, 1H), 4.19-3.83 (m, 4H), 3.79 (s, 3H), 3.74-3.63 (m, 1H), 3.51-3.35 (m, 2H), 1.42 (s, 9H).

LCMS Condition P: Rt=1.66 min. m/z 280.34 [M+H]⁺.

### Step-6—Preparation of tert-butyl 4,4-difluoro-3-(1-methyl-1H-pyrazol-4-yl)piperidine-1-carboxylate

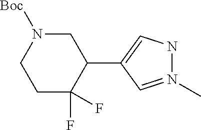

To a stirred solution of tert-butyl 3-(1-methyl-1H-pyrazol-4-yl)-4-oxopiperidine-1-carboxylate (110 mg, 0.39 mmol) in DCM (5.0 mL) at 0° C. was added DAST (0.11 ml, 0.79 mmol). The resulting mixture was stirred at RT for 16 h. The reaction was quenched with water, extracted with ethyl acetate and concentrated under reduced pressure. The crude product was purified by column chromatography (40% EA in Hexane) to afford tert-butyl 4,4-difluoro-3-(1-methyl-1H-pyrazol-4-yl)piperidine-1-carboxylate (50 mg, 42.3% yield) as a colourless gum.

1H NMR (400 MHz, DMSO-d6) δ 7.61 (s, 1H), 7.34 (s, 1H), 3.97-3.85 (m, 2H), 3.80 (s, 3H), 3.25-2.99 (m, 3H), 2.21-1.81 (m, 2H), 1.40 (s, 9H).

LCMS Condition N: Rt=1.95 min. m/z 302.4 [M+H]⁺.

### Step-7—Preparation of 4,4-difluoro-3-(1-methyl-1H-pyrazol-4-yl)piperidine

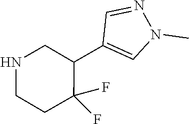

Dioxane-HCl (4M, 2 mL, 8 mmol) was added to tert-butyl 4,4-difluoro-3-(1-methyl-1H-pyrazol-4-yl)piperidine-1-carboxylate (40 mg, 0.13 mmol) at 0° C. Resulting mixture was warmed to ambient temperature and stirred for 4 h. After completion, The reaction mixture was concentrated under reduced pressure, triturated with pentane to afford 4,4-difluoro-3-(1-methyl-1H-pyrazol-4-yl)piperidine (30 mg, 89.4% yield, HCl salt) as off white solid.

1H NMR (400 MHz, DMSO-d6) δ 9.76-9.74 (br s, 1H), 9.50-9.45 (br s, 1H), 7.72 (s, 1H), 7.40 (s, 1H), 3.82 (s, 3H), 3.68-3.60 (m, 1H), 3.54-3.40 (m, 2H), 3.30-2.95 (m, 2H), 2.45-2.28 (m, 2H) [HCl salt].

LCMS Condition N: Rt=0.29 min. m/z 202.3 [M+H]⁺.

### Step-8—Preparation of 8-(4-chloro-2-fluorophenyl)-6-(4,4-difluoro-3-(1-methyl-1H-pyrazol-4-yl)piperidin-1-yl)-2,3-dimethylpyrimido[5,4-d]pyrimidin-4 (3H)-one

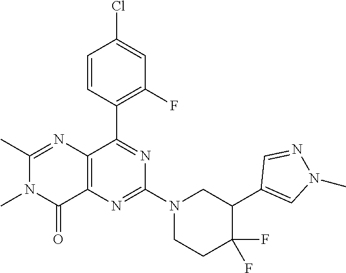

Procedure of Step-5, example-1 was followed using 6 6-chloro-8-(4-chloro-2-fluorophenyl)-2,3-dimethylpyrimido[5,4-d]pyrimidin-4 (3H)-one and 4,4-difluoro-3-(1-methyl-1H-pyrazol-4-yl)piperidine hydrochloride salt as starting materials followed by reverse phase prep-HPLC purification to afford 8-(4-chloro-2-fluorophenyl)-6-(4,4-difluoro-3-(1-methyl-1H-pyrazol-4-yl)piperidin-1-yl)-2,3-dimethylpyrimido[5,4-d]pyrimidin-4 (3H)-one as yellow solid in 44.7% yield.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

1H NMR (400 MHz, DMSO-d6) δ 7.73-7.57 (m, 3H), 7.46 (d, J=8.2 Hz, 1H), 7.38 (s, 1H), 4.90-4.65 (m, 2H), 3.81 (s, 3H), 3.51 (s, 3H), 3.49-3.32 (m, 2H), 3.28-3.20 (m, 1H), 2.44 (s, 3H), 2.31-1.96 (m, 2H).

LCMS Condition M: Rt=3.00 min. m/z 504.38 [M+H]⁺.

### Example 14

### 8-(4-chloro-2-fluorophenyl)-6-((2R,4S)-2-(1-cyclopropyl-1H-pyrazol-4-yl)tetrahydro-2H-pyran-4-yl)-2,3-dimethylpyrimido[5,4-d]pyrimidin-4 (3H)-one

### Step-1—Preparation of 6-(1-cyclopropyl-1H-pyrazol-4-yl)-3,6-dihydro-2H-pyran-4-yl trifluoromethanesulfonate

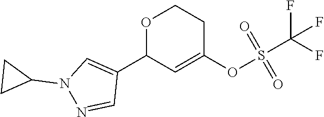 [SMILES: O=S(=O)(OC1=CC(c2cnn(C3CC3)c2)OCC1)C(F)(F)F]

To a stirred solution of 1-cyclopropyl-1H-pyrazole-4-carbaldehyde (300 mg, 2.21 mmol) in DCM (5.0 mL) at 0° C. was added but-3-yn-1-ol (0.277 mL, 3.31 mmol) followed by addition of Triflic acid (0.47 mL, 5.3 mmol). Resulting mixture was stirred at RT for 16 h. The reaction was quenched with saturated aqueous sodium bicarbonate solution, extracted with DCM and concentrated under reduced pressure. The crude material was purified by column chromatography (20-40% EA in Hexane) to afford 6-(1-cyclopropyl-1H-pyrazol-4-yl)-3,6-dihydro-2H-pyran-4-yl trifluoromethanesulfonate (270.0 mg, 36.4% yield) as sticky liquid.

1H NMR (400 MHz, DMSO-d6) δ 7.74 (s, 1H), 7.36 (s, 1H), 6.20 (s, 1H), 5.32 (s, 1H), 3.91-3.64 (m, 3H), 2.49-2.42 (m, 2H), 1.11-0.88 (m, 4H).

LCMS Condition Q: Rt=1.82 min. m/z 339.1 [M+H]⁺.

### Step-2—Preparation of 1-cyclopropyl-4-(4-(4,4,5,5-tetramethyl-1,3,2-dioxaborolan-2-yl)-5,6-dihydro-2H-pyran-2-yl)-1H-pyrazole

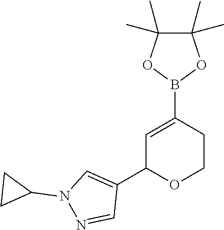 [SMILES: CC1(C)OB(C2=CC(c3cnn(C4CC4)c3)OCC2)OC1(C)C]

To a stirred solution of 6-(1-cyclopropyl-1H-pyrazol-4-yl)-3,6-dihydro-2H-pyran-4-yl trifluoromethanesulfonate (270.0 mg, 0.799 mmol) and Bis(pinacolato)diboron (304.229 mg, 1.198 mmol) in dioxane (5 mL) was added potassium acetate (313.456 mg, 3.195 mmol) and degassed with argon. PdCl2(dppf)·DCM (65.235 mg, 0.08 mmol) was added under inert atmosphere. The resulting mixture was heated at 80° C. for 3 h. The reaction mixture was diluted with ethyl acetate, filtered through a short pad of celite and washed with ethyl acetate. The combined organic layers were washed with water, brine, dried over anhydrous sodium sulphate, filtered and concentrated under reduced pressure. The crude product was purified by column chromatography (30% EA in Hexane) to afford 1-cyclopropyl-4-(4-(4,4,5,5-tetramethyl-1,3,2-dioxaborolan-2-yl)-5,6-dihydro-2H-pyran-2-yl)-1H-pyrazole (190 mg, 75.2% yield) as gum.

1H NMR (400 MHz, DMSO-d6) δ 7.67 (s, 1H), 7.31 (s, 1H), 6.43 (d, J=2.2 Hz, 1H), 5.16-5.01 (m, 1H), 3.82-3.63 (m, 2H), 3.63-3.53 (m, 1H), 2.20-2.00 (m, 2H), 1.15-0.96 (m, 16H).

LCMS Condition O: Rt=3.25 min. m/z 316.9 [M+H]⁺.

### Step-3—Preparation of 8-(4-chloro-2-fluorophenyl)-6-(6-(1-cyclopropyl-1H-pyrazol-4-yl)-3,6-dihydro-2H-pyran-4-yl)-2,3-dimethylpyrimido[5,4-d]pyrimidin-4 (3H)-one

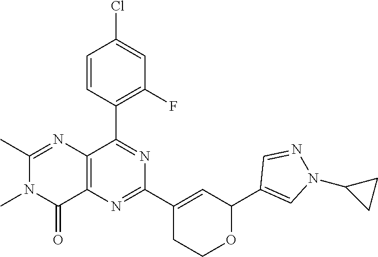 [SMILES: Cc1nc2c(-c3ccc(Cl)cc3F)nc(C3=CC(c4cnn(C5CC5)c4)OCC3)nc2c(=O)n1C]

To a stirred solution of 6-chloro-8-(4-chloro-2-fluorophenyl)-2,3-dimethylpyrimido[5,4-d]pyrimidin-4 (3H)-one (50.0 mg, 0.15 mmol) and 1-cyclopropyl-4-(4-(4,4,5,5-tetramethyl-1,3,2-dioxaborolan-2-yl)-5,6-dihydro-2H-pyran-2-yl)-1H-pyrazole (186.9 mg, 0.59 mmol) in dioxane (4 mL) and water (1 mL) was added potassium carbonate (40.8 mg, 0.29 mmol) and degassed with argon. PdCl2(dppf)·DCM (12.1 mg, 0.015 mmol) was added under inert atmosphere. Resulting mixture was heated at 80° C. for 3 h. The reaction mixture was diluted with ethyl acetate, filtered through a short pad of celite and washed with ethyl acetate. The combined organic layers were washed with water, brine, dried over anhydrous sodium sulphate, filtered and concentrated under reduced pressure. The crude product was purified by column chromatography (70% EA in Hexane) to afford 8-(4-chloro-2-fluorophenyl)-6-(6-(1-cyclopropyl-1H-pyrazol-4-yl)-3,6-dihydro-2H-pyran-4-yl)-2,3-dimethylpyrimido[5,4-d]pyrimidin-4 (3H)-one (32 mg, 43.2% yield) as off-white solid.

1H NMR (400 MHz, DMSO-d6) δ 7.76 (s, 1H), 7.71 (t, J=8.0 Hz, 1H), 7.64 (dd, J=1.9, 9.9 Hz, 1H), 7.48 (dd, J=2.0, 8.3 Hz, 1H), 7.40 (s, 1H), 7.28 (s, 1H), 5.40 (s, 1H), 4.04-3.92 (m, 1H), 3.85-3.75 (m, 1H), 3.73-3.63 (m, 1H), 3.56 (s, 3H), 2.81-2.63 (m, 2H), 2.50 (s, 3H), 0.93-0.83 (m, 4H).

LCMS Condition N: Rt=2.14 min. m/z 493.4 [M+H]⁺.

### Step-4—Preparation of cis-racemate-8-(4-chloro-2-fluorophenyl)-6-((2R,4S)-2-(1-cyclopropyl-1H-pyrazol-4-yl)tetrahydro-2H-pyran-4-yl)-2,3-dimethylpyrimido[5,4-d]pyrimidin-4 (3H)-one

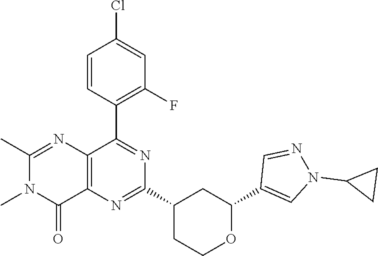 [SMILES: Cc1nc2c(-c3ccc(Cl)cc3F)nc([C@H]3CCO[C@@H](c4cnn(C5CC5)c4)C3)nc2c(=O)n1C]

Procedure of Step-3, example-11 was followed using afford 8-(4-chloro-2-fluorophenyl)-6-(6-(1-cyclopropyl-1H-pyrazol-4-yl)-3,6-dihydro-2H-pyran-4-yl)-2,3-dimethylpyrimido[5,4-d]pyrimidin-4 (3H)-one as starting material followed by purification by reverse phase prep HPLC to afford 8-(4-chloro-2-fluorophenyl)-6-((2R,4S)-2-(1-cyclopropyl-1H-pyrazol-4-yl)tetrahydro-2H-pyran-4-yl)-2,3-dimethylpyrimido[5,4-d]pyrimidin-4 (3H)-one as cis racemate as white solid in 26.5% yield.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 50% A and 50% B, then 40% A and 60% B in 3 min, then to 30% A and 70% B in 20 min., then to 5% A and 95% B in 21 min., held this composition up to 22 min. for column washing, then returned to initial composition in 23 min. and held till 25 min.

1H NMR (400 MHz, DMSO-d6) δ 7.75-7.61 (m, 3H), 7.49 (dd, J=2.0, 8.3 Hz, 1H), 7.36 (s, 1H), 4.49 (d, J=10.6 Hz, 1H), 4.08 (dd, J=4.1, 11.0 Hz, 1H), 3.78-3.59 (m, 2H), 3.55 (s, 3H), 3.47-3.35 (m, 1H), 2.52 (s, 3H), 2.22 (d, J=13.1 Hz, 1H), 2.02-1.78 (m, 3H), 1.02-0.96 (m, 2H), 0.95-0.87 (m, 2H).

LCMS Condition M: Rt=2.87 min. m/z 495.39 [M+H]⁺.

### Example 15

### 8-(4-chloro-2-fluorophenyl)-2,3-dimethyl-6-(3-(((1-methyl-1H-pyrazol-4-yl)oxy)methyl)azetidin-1-yl)pyrimido[5,4-d]pyrimidin-4 (3H)-one

### Step-1—Preparation of tert-butyl 3-(((1-methyl-1H-pyrazol-4-yl)oxy)methyl)azetidine-1-carboxylate

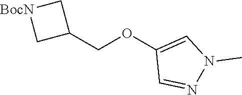 [SMILES: Cn1cc(OCC2CN(C(=O)OC(C)(C)C)C2)cn1]

To a stirred solution of 1-methyl-1H-pyrazol-4-ol (600 mg, 6.11 mmol) in DMF (20 mL) at 0° C., was added NaH (293.5 mg, 12.22 mmol) and the reaction mixture was stirred for 15 min. Then, tert-butyl 3-(bromomethyl)azetidine-1-carboxylate (1.5 g, 6.11 mmol) was added to the reaction mixture and heated at 60° C. for 4 h. Reaction mixture was cooled to RT, diluted with water and extracted with ethyl acetate. The combined organic layer was washed with bine, dried over anhydrous Na₂SO₄, filtered and concentrated under reduced pressure. The crude residue was purified by CombiFlash column chromatography (0 to 50% EtOAc-hexane) to afford tert-butyl 3-(((1-methyl-1H-pyrazol-4-yl)oxy)methyl)azetidine-1-carboxylate (800 mg, 48.9% yield) as a white solid.

¹H NMR (400 MHz, DMSO D) δ 7.44 (s, 1H), 7.17 (s, 1H), 3.98-3.93 (m, 4H), 3.72 (s, 3H), 3.62-3.59 (m, 2H), 2.89-2.86 (m, 1H), 1.38 (s, 9H).

LCMS Condition K: Rt=1.67 min. m/z 268.0 [M+H]⁺.

### Step-2—Preparation of 4-(azetidin-3-ylmethoxy)-1-methyl-1H-pyrazole hydrochloride

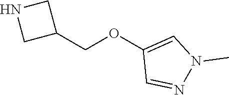

To a stirred solution of tert-butyl 3-(((1-methyl-1H-pyrazol-4-yl)oxy)methyl)azetidine-1-carboxylate (800 mg, 2.996 mmol) in 1,4-Dioxane (16 mL) at 0° C. was added 4M HCl in 1,4-dioxane (8 mL). Reaction mixture was slowly warmed to RT and stirred for 2 h. Volatiles were removed under reduced pressure to afford 4-(azetidin-3-ylmethoxy)-1-methyl-1H-pyrazole hydrochloride (450 mg, 74% yield) as a gummy solid.

LCMS Condition J: Rt=0.29 min. m/z 167.9 [M+H]⁺.

### Step-3—Preparation of 8-(4-chloro-2-fluorophenyl)-2,3-dimethyl-6-(3-(((1-methyl-1H-pyrazol-4-yl)oxy)methyl)azetidin-1-yl)pyrimido[5,4-d]pyrimidin-4 (3H)-one

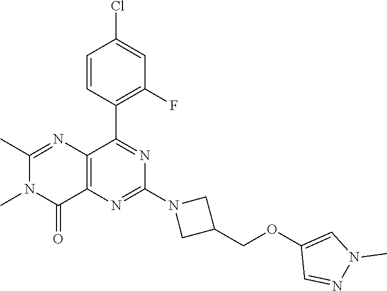

To a stirred solution of 6-chloro-8-(4-chloro-2-fluorophenyl)-2,3-dimethylpyrimido[5,4-d]pyrimidin-4 (3H)-one (80 mg, 0.24 mmol) and 4-(azetidin-3-ylmethoxy)-1-methyl-1H-pyrazole hydrochloride (79 mg, 0.47 mmol) in DMSO (2 mL) was added DIPEA (0.06 mL, 0.47 mmol). Resulting mixture was heated at 100° C. for 12 h. After completion, reaction mixture was concentrated under reduced pressure and The crude product was purified by reverse phase preparative HPLC to afford 8-(4-chloro-2-fluorophenyl)-2,3-dimethyl-6-(3-(((1-methyl-1H-pyrazol-4-yl)oxy)methyl)azetidin-1-yl)pyrimido[5,4-d]pyrimidin-4 (3H)-one (40 mg, 35.9% yield) as a white solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 35% A and 65% B in 3 min, then to 10% A and 90% B in 20 min., then to 5% A and 95% B in 21 min., held this composition up to 22 min. for column washing, then returned to initial composition in 23 min. and held till 25 min.

¹H NMR (400 MHz, DMSO D₆) δ 7.61-5.80 (m, 2H), 7.47-7.44 (m, 2H), 7.19 (s, 1H), 4.24 (t, J=8.2 Hz, 2H), 4.06 (d, J=6.4 Hz, 2H), 3.96-3.92 (m, 2H), 3.72 (s, 3H), 3.49 (s, 3H), 3.13-3.10 (m, 1H), 2.42 (s, 3H).

LCMS Condition J: Rt=2.80 min. m/z 470.3 [M+H]⁺.

### Example 16

### 8-(4-chloro-2-fluorophenyl)-2,3-dimethyl-6-(3-((1-methyl-1H-pyrazol-4-yl)oxy)azetidin-1-yl)pyrimido[5,4-d]pyrimidin-4 (3H)-one

### Step-1—Preparation of tert-butyl 3-((1-methyl-1H-pyrazol-4-yl)oxy)azetidine-1-carboxylate

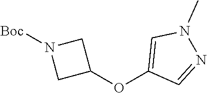 [SMILES: Cn1cc(OC2CN(C(=O)OC(C)(C)C)C2)cn1]

To a stirred solution of 1-methyl-1H-pyrazol-4-ol (200.0 mg, 2.04 mmol) in NMP (5 mL) were added cesium carbonate (1.33 g, 4.08 mmol) and tert-butyl 3-iodoazetidine-1-carboxylate (578.0 mg, 2.04 mmol) at RT. Resulting mixture was heated at 60° C. for 16 h. After completion, reaction mixture was diluted with cold water and extracted with ethyl acetate. Combine organic layer was washed with brine, dried over sodium sulfate, filtered and concentrated under reduced pressure. The crude product was purified by column chromatography (50% EtOAc-hexane) to afford tert-butyl 3-((1-methyl-1H-pyrazol-4-yl)oxy)azetidine-1-carboxylate (120.0 mg, 23.2% yield) as a colourless gum.

LCMS Condition I: Rt=1.73 min. m/z 254.2 [M+H]⁺.

### Step-2—Preparation of 4-(azetidin-3-yloxy)-1-methyl-1H-pyrazole

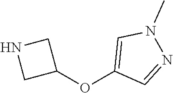

To a stirred solution of tert-butyl 3-((1-methyl-1H-pyrazol-4-yl)oxy)azetidine-1-carboxylate (100 mg, 0.4 mmol) in dry DCM (3 mL) at 0° C. was added TFA (1.0 mL). The reaction mixture was slowly warmed to RT and stirred for 2 h. After completion, volatiles were removed under reduced pressure and the residue was triturated with diethyl ether/pentane (1:1) to afford 4-(azetidin-3-yloxy)-1-methyl-1H-pyrazole (60.0 mg, 99.22% yield, TFA salt) as a colourless gum.

LCMS Condition I: Rt=0.29 min. m/z 154.2 [M+H]⁺.

### Step-3—Preparation of 8-(4-chloro-2-fluorophenyl)-2,3-dimethyl-6-(3-((1-methyl-1H-pyrazol-4-yl)oxy)azetidin-1-yl)pyrimido[5,4-d]pyrimidin-4 (3H)-one

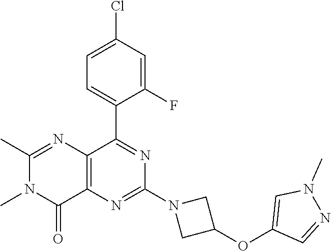 [SMILES: Cc1nc2c(-c3ccc(Cl)cc3F)nc(N3CC(Oc4cnn(C)c4)C3)nc2c(=O)n1C]

To a stirred solution of 6-chloro-8-(4-chloro-2-fluorophenyl)-2,3-dimethylpyrimido[5,4-d]pyrimidin-4 (3H)-one (50.0 mg, 0.147 mmol) in DMF (2 mL) were added 4-(azetidin-3-yloxy)-1-methyl-1H-pyrazole (58.84 mg, 0.22 mmol; TFA salt) and K₂CO₃ (61.12 mg, 0.44 mmol). Resulting mixture was heated at 70° C. for 16 h. After completion, reaction mixture was diluted with cold water and extracted with 5% MeoH-DCM. Combine organic layer was dried over sodium sulphate, filtered and concentrated under reduced pressure. The crude product was purified by prep-HPLC to get 8-(4-chloro-2-fluorophenyl)-2,3-dimethyl-6-(3-((1-methyl-1H-pyrazol-4-yl)oxy)azetidin-1-yl)pyrimido[5,4-d]pyrimidin-4 (3H)-one (25.0 mg, 37.2% yield) as a yellow solid. Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 45% A and 55% B in 3 min, then to 25% A and 75% B in 20 min., then to 5% A and 95% B in 21 min., held this composition up to 22 min. for column washing, then returned to initial composition in 23 min. and held till 25 min.

¹H NMR (400 MHz, DMSO D6) 67.64-7.59 (m, 2H), 7.51 (s, 1H), 7.46 (d, J=7.92 Hz, 1H), 7.21 (s, 1H), 4.88 (bs, 1H), 4.52 (t, J=8.28 Hz, 2H), 4.07 (d, J=8.44 Hz, 2H), 3.74 (s, 3H), 3.51 (s, 3H), 2.43 (s, 3H).

LCMS Condition I: Rt=2.82 min. m/z 456.35 [M+H]⁺.

### Example 17

### 8-(4-chloro-2-fluorophenyl)-2,3-dimethyl-6-(2-(1-methyl-1H-pyrazol-4-yl)tetrahydro-2H-pyran-4-yl)pyrimido[5,4-d]pyrimidin-4 (3H)-one

### Step-1—Preparation of 6-(1-methyl-1H-pyrazol-4-yl)-3,6-dihydro-2H-pyran-4-yl trifluoromethanesulfonate

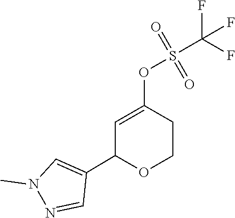 [SMILES: Cn1cc(C2C=C(OS(=O)(=O)C(F)(F)F)CCO2)cn1]

To a stirred solution of 1-methyl-1H-pyrazole-4-carbaldehyde (1 gm, 9.09 mmol) in dichloromethane (20 ml) was added but-3-yn-1-ol (1.03 ml, 13.64 mmol) under argon atmosphere. Reaction mixture was cooled to 0° C. and trifluoromethanesulfonic acid (0.96 ml, 10.91 mmol) was added drop wise. Resulting mixture was warmed to RT and stirred for 5 h. Trifluoromethanesulfonic acid (0.96 ml, 10.91 mmol) was added again at 0° C. and stirred at RT for 16 hours. Reaction mixture was quenched with saturated NaHCO₃ solution and extracted with dichloromethane. The combined organic layer was dried over anhydrous sodium sulphate, filtered and concentrated under reduced pressure. The crude product was purified by combi flash column chromatography (20-50% ethyl acetate in hexane) to afford 6-(1-methyl-1H-pyrazol-4-yl)-3,6-dihydro-2H-pyran-4-yl trifluoromethanesulfonate (1.2 gm, 42.3% yield) as colorless liquid.

1H NMR (400 MHz, CDCl3) δ 7.46 (s, 1H), 7.34 (s, 1H), 5.93 (s, 1H), 5.31 (d, J=1.84 Hz, 1H), 4.00-3.95 (m, 1H), 3.89 (s, 3H), 3.84-3.79 (m, 1H), 2.55-2.44 (m, 2H).

LCMS Condition C: Rt=3.02 min. m/z 313 [M+H]⁺.

### Step-2—Preparation of 1-methyl-4-(4-(4,4,5,5-tetramethyl-1,3,2-dioxaborolan-2-yl)-5,6-dihydro-2H-pyran-2-yl)-1H-pyrazole

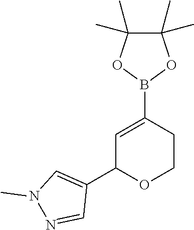

To a stirred solution of 6-(1-methyl-1H-pyrazol-4-yl)-3,6-dihydro-2H-pyran-4-yl trifluoromethanesulfonate (700 mg, 2.24 mmol) in dioxane (10 mL) were added 4,4,5,5-tetramethyl-2-(4,4,5,5-tetramethyl-1,3,2-dioxaborolan-2-yl)-1,3,2-dioxaborolane (854.47 mg, 3.36 mmol) and potassium acetate (880.38 mg, 8.97 mmol) and thoroughly purged with argon. PdCl2(dppf). DCM (183.22 mg, 0.22 mmol) was added under inert atmosphere. Resulting mixture was heated at 90° C. for 2 h. Reaction mixture was filtered through a short pad of celite and washed with ethyl acetate. Filtrate was washed with water, brine, dried over sodium sulphate, filtered and concentrated under reduced pressure. The crude material was purified by column chromatography (10-30% ethyl acetate/hexane) to afford 1-methyl-4-(4-(4,4,5,5-tetramethyl-1,3,2-dioxaborolan-2-yl)-5,6-dihydro-2H-pyran-2-yl)-1H-pyrazole (300 mg, 46.1% yield) as colorless liquid.

1H NMR (400 MHz, DMSO-d6) δ 7.58 (s, 1H), 7.32 (s, 1H), 6.45 (s, 1H), 5.11 (s, 1H), 3.79 (s, 3H), 3.74-3.71 (m, 1H), 3.61-3.57 (m, 1H), 2.07-1.99 (m, 2H), 1.21 (s, 12H).

LCMS Condition C: Rt=2.96 min. m/z 291.1 [M+H]⁺.

### Step-3—Preparation of 8-(4-chloro-2-fluorophenyl)-2,3-dimethyl-6-(6-(1-methyl-1H-pyrazol-4-yl)-3,6-dihydro-2H-pyran-4-yl)pyrimido[5,4-d]pyrimidin-4 (3H)-one

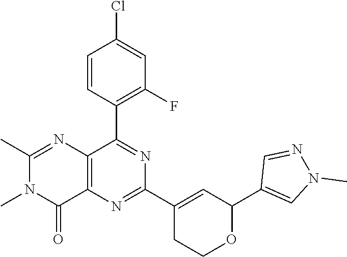

To a stirred solution of 6-chloro-8-(4-chloro-2-fluorophenyl)-2,3-dimethylpyrimido[5,4-d]pyrimidin-4 (3H)-one (150 mg, 0.44 mmol) and 1-methyl-4-(4-(4,4,5,5-tetramethyl-1,3,2-dioxaborolan-2-yl)-5,6-dihydro-2H-pyran-2-yl)-1H-pyrazole (193.047 mg, 0.666 mmol) in dioxane (4 mL) and water (1 mL) was added cesium carbonate (432.69 mg, 1.33 mmol) and degassed with argon. PdCl2(dpp). DCM (36.21 mg, 0.04 mmol) was added under inert atmosphere. Resulting mixture was heated at 70° C. for 2 h. Reaction mixture was diluted with ethyl acetate, filtered through a short pad of celite and washed with ethyl acetate. The combined organic layers were washed with water, brine, dried over anhydrous sodium sulphate, filtered and concentrated under reduced pressure. The crude product was purified by column chromatography (35% ethyl acetate-hexane) to afford 8-(4-chloro-2-fluorophenyl)-2,3-dimethyl-6-(6-(1-methyl-1H-pyrazol-4-yl)-3,6-dihydro-2H-pyran-4-yl)pyrimido[5,4-d]pyrimidin-4 (3H)-one (130 mg, 62.7% yield).

1H NMR (400 MHz, DMSO-d6) δ 7.73-7.63 (m, 3H), 7.49 (d, J=8.2 Hz, 1H), 7.40 (s, 1H), 7.30 (s, 1H), 5.41 (s, 1H), 3.98-3.95 (m, 1H), 3.82-3.81 (m, 1H), 3.79 (s, 3H), 3.56 (s, 3H), 2.71-2.67 (m, 2H), 2.53 (s, 3H).

LCMS Condition B: Rt=2.02 min. m/z 467.3 [M+H]⁺.

### Step-4—Preparation of 8-(4-chloro-2-fluorophenyl)-2,3-dimethyl-6-(2-(1-methyl-1H-pyrazol-4-yl)tetrahydro-2H-pyran-4-yl)pyrimido[5,4-d]pyrimidin-4 (3H)-one

### Example-5

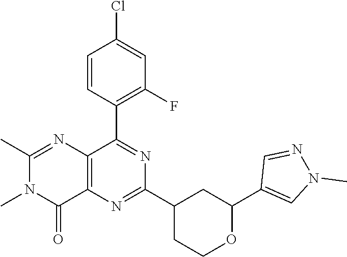

To a degassed solution of 8-(4-chloro-2-fluorophenyl)-2,3-dimethyl-6-(6-(1-methyl-1H-pyrazol-4-yl)-3,6-dihydro-2H-pyran-4-yl)pyrimido[5,4-d]pyrimidin-4 (3H)-one (130 mg, 0.28 mmol) in ethanol (15 mL), 10% Pd—C (50% wet, 60 mg) was added. Resulting mixture was stirred at RT under hydrogen at balloon pressure for 16 h. Reaction mixture was filtered through a short pad of celite, washed with ethanol. The combined filtrate was concentrated under reduced pressure and purified by reverse phase prep HPLC to afford 8-(4-chloro-2-fluorophenyl)-2,3-dimethyl-6-(2-(1-methyl-1H-pyrazol-4-yl)tetrahydro-2H-pyran-4-yl)pyrimido[5,4-d]pyrimidin-4 (3H)-one (16 mg, 12.2% yield). Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 50% A and 50% B in 3 min, then to 40% A and 60% B in 20 min., then to 5% A and 95% B in 21 min., held this composition up to 22 min. for column washing, then returned to initial composition in 23 min. and held till 25 min.

1H NMR (400 MHz, DMSO-d6) δ 7.70 (t, J=8.0 Hz, 1H), 7.66-7.61 (m, 2H), 7.49 (dd, J=2.0, 8.3 Hz, 1H), 7.36 (s, 1H), 4.51-4.48 (m, 1H), 4.10-4.07 (m, 1H), 3.78 (s, 3H), 3.72-3.66 (m, 1H), 3.56 (s, 3H), 3.45-3.39 (m, 1H), 2.52 (s, 3H), 2.23-2.20 (m, 1H), 2.07-1.87 (m, 3H).

LCMS Condition E: Rt=1.79 min. m/z 469.3 [M+H]⁺.

### Example 18

### 8-(4-chloro-2-fluorophenyl)-2,3-dimethyl-6-(3-((1-methyl-1H-pyrazol-4-yl)methoxy)azetidin-1-yl)pyrimido[5,4-d]pyrimidin-4 (3H)-one

### Step-1—Preparation of 4-(chloromethyl)-1-methyl-1H-pyrazole hydrochloride

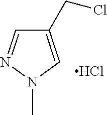

To a stirred solution of (1-methyl-1H-pyrazol-4-yl)methanol (1.0 g, 8.93 mmol) in DCM at 0° C., was added SOCl₂ (2.0 mL, 27.56 mmol) and the resulting reaction mixture was stirred at RT for 3 h. Volatiles were removed under reduced pressure and crude residue was triturated with diethyl ether/pentane (1:1; two times) to afford 4-(chloromethyl)-1-methyl-1H-pyrazole hydrochloride (800 mg; 68.62% yield; HCl salt) as a white solid.

¹H NMR (400 MHz, DMSO D) δ 9.91-9.72 (brs, 1H), 7.78 (s, 1H), 7.47 (s, 1H), 4.68 (s, 2H), 3.80 (s, 3H).

### Step-2—Preparation of tert-butyl 3-((1-methyl-1H-pyrazol-4-yl)methoxy)azetidine-1-carboxylate

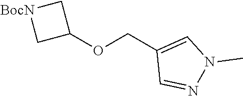 [SMILES: Cn1cc(COC2CN(C(=O)OC(C)(C)C)C2)cn1]

To a stirred solution of tert-butyl 3-hydroxyazetidine-1-carboxylate (500 mg, 3.85 mmol) in dry DMF (10 mL) at 0° C. was added sodium hydride (184.62 mg, 7.7 mmol). Resulting suspension was stirred for 30 min and 4-(chloromethyl)-1-methyl-1H-pyrazole (666.2 mg, 3.85 mmol) was added in a portion. Resulting mixture was heated at 60° C. for 6 h. After completion, reaction mixture was cooled to ambient temperature, diluted with cold water and extracted with ethyl acetate. Combined organic layer was washed with brine, dried over anhydrous sodium sulphate, filtered and concentrated under reduced pressure. Crude mass was purified by column chromatography (50% EA-hexane) to afford tert-butyl 3-((1-methyl-1H-pyrazol-4-yl)methoxy)azetidine-1-carboxylate (300.0 mg, 29.2% yield) as a colourless gum.

¹H NMR (400 MHz, DMSO D) 67.67 (s, 1H), 7.38 (s, 1H), 4.27 (s, 2H), 4.28-4.25 (m, 1H), 3.98-3.95 (m, 2H), 3.79 (s, 3H), 3.62-3.60 (m, 2H), 1.37 (s, 9H).

LCMS Condition I: Rt=1.70 min. m/z 268.0 [M+H]⁺.

### Step-3—Preparation of 4-((azetidin-3-yloxy)methyl)-1-methyl-1H-pyrazole TFA Salt

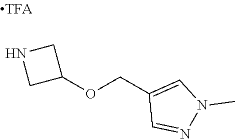

To a stirred solution of tert-butyl 3-((1-methyl-1H-pyrazol-4-yl)methoxy)azetidine-1-carboxylate (300 mg, 1.12 mmol) in DCM (6 mL) at 0° C. was added TFA (0.3 mL). Reaction mixture was slowly warmed up to RT and stirred for 2 h. After completion, volatiles were removed under reduced pressure and the residue was triturated with diethyl ether/pentane (1:1) to afford 4-(azetidin-3-yloxy)-1-methyl-1H-pyrazole (250 mg crude, TFA salt) as a colourless gum.

LCMS Condition I: Rt=0.24 min. m/z 168.1 [M+H]⁺.

### Step-4—Preparation of 8-(4-chloro-2-fluorophenyl)-2,3-dimethyl-6-(3-((1-methyl-1H-pyrazol-4-yl)methoxy)azetidin-1-yl)pyrimido[5,4-d]pyrimidin-4 (3H)-one

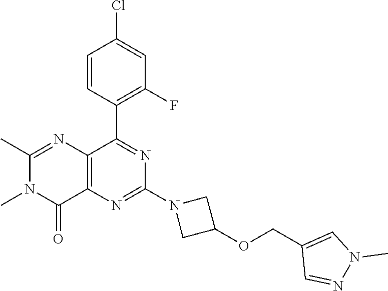

To a solution of 6-chloro-8-(4-chloro-2-fluorophenyl)-2,3-dimethylpyrimido[5,4-d]pyrimidin-4 (3H)-one (80.0 mg, 0.236 mmol) and 4-((azetidin-3-yloxy)methyl)-1-methyl-1H-pyrazole (80 mg, 0.470 mmol; TFA salt) in DMF (2 mL) at rt was added K₂CO₃ (65 mg, 0.472 mmol). The resulting suspension was heated at 50° C. for 16 h. After completion, the reaction mixture was diluted with cold water and extracted with 5% MeOH-DCM. Combine organic layer was washed with brine, dried over sodium sulfate, filtered and concentrated under reduced pressure. Crude product was purified by reverse phase preparative HPLC to 8-(4-chloro-2-fluorophenyl)-2,3-dimethyl-6-(3-((1-methyl-1H-pyrazol-4-yl)methoxy)azetidin-1-yl)pyrimido[5,4-d]pyrimidin-4 (3H)-one (80 mg, 72.8%) as a yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 50% A and 50% B in 3 min, then to 20% A and 80% B in 20 min., then to 5% A and 95% B in 21 min., held this composition up to 22 min. for column washing, then returned to initial composition in 23 min. and held till 25 min.

1H NMR (400 MHz, DMSO D6) δ 7.72 (s, 1H), 7.59 (d, J=8.4 Hz, 2H), 7.46-7.42 (m, 2H), 4.48-4.46 (m, 1H), 4.37 (s, 2H), 4.31 (t, J=6.0 Hz, 2H), 3.91 (d, J=6.0 Hz, 2H), 3.81 (s, 3H), 3.5 (s, 3H), 2.42 (s, 3H).

LCMS Condition J: Rt=2.803 min. m/z 470.28 [M+H]⁺.

Examples 19A-19B to Examples 28A-28B were synthesized by chiral separation of racemate compound:

### Example 19

### Example-19A: Enantiomer 1

### (R)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one

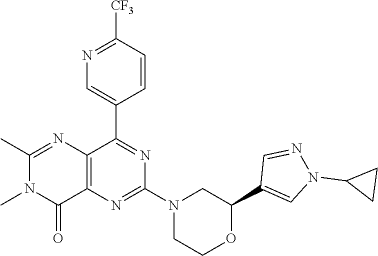 [SMILES: Cc1nc2c(-c3ccc(C(F)(F)F)nc3)nc(N3CCO[C@H](c4cnn(C5CC5)c4)C3)nc2c(=O)n1C]

### Example-19B: Enantiomer 2

### (S)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one

### Step-1—Preparation of 6-(2-(1H-pyrazol-4-yl)morpholino)-2,3-dimethyl-8-(6-(trifluoromethyl)pyridin-3-yl)pyrimido[5,4-d]pyrimidin-4 (3H)-one

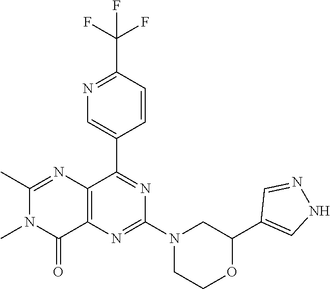 [SMILES: Cc1nc2c(-c3ccc(C(F)(F)F)nc3)nc(N3CCOC(c4cn[nH]c4)C3)nc2c(=O)n1C]

General procedure of Step-2, example-4 to 10 was followed using compounds 3a [6-chloro-2,3-dimethyl-8-(6-(trifluoromethyl)pyridin-3-yl)pyrimido[5,4-d]pyrimidin-4 (3H)-one] and 2-(1H-pyrazol-4-yl)morpholine [synthesized in example-2, step-7] as starting materials to afford 6-(2-(1H-pyrazol-4-yl)morpholino)-2,3-dimethyl-8-(6-(trifluoromethyl)pyridin-3-yl)pyrimido[5,4-d]pyrimidin-4 (3H)-one as yellow solid in 63.4% yield.

1H NMR (400 MHz, DMSO D6) δ 12.86 (br s, 1H), 9.59 (s, 1H), 8.91 (d, J=7.9 Hz, 1H), 8.09 (d, J=8.2 Hz, 1H), 7.81 (s, 1H), 7.56 (s, 1H), 4.72-4.56 (m, 3H), 4.02-3.88 (m, 1H), 3.71-3.65 (m, 1H), 3.54 (s, 3H), 3.33 (m, 2H), 2.54 (s, 3H).

LCMS Condition F: Rt=1.74 min, m/z 473.2 [M+H]+.

### Step-2—Preparation of 6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one

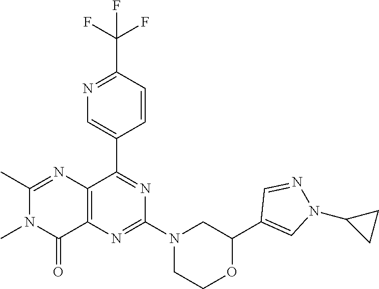 [SMILES: Cc1nc2c(-c3ccc(C(F)(F)F)nc3)nc(N3CCOC(c4cnn(C5CC5)c4)C3)nc2c(=O)n1C]

Procedure of Step-9, example-2 was followed using compound 6-(2-(1H-pyrazol-4-yl)morpholino)-2,3-dimethyl-8-(6-(trifluoromethyl)pyridin-3-yl)pyrimido[5,4-d]pyrimidin-4 (3H)-one as starting material to afford 6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one as yellow solid in 38.6% yield.

### Example 20

### Example-20A: Enantiomer 1

### (R)-6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one

### Example-20B: Enantiomer 2

### (S)-6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one

### Step-1—Preparation of 6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one

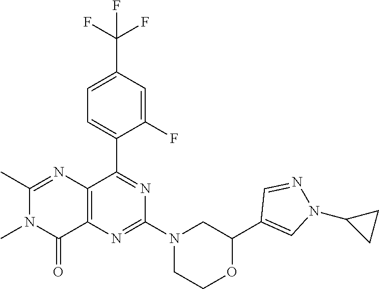 [SMILES: Cc1nc2c(-c3ccc(C(F)(F)F)cc3F)nc(N3CCOC(c4cnn(C5CC5)c4)C3)nc2c(=O)n1C]

General procedure of Step-2, example-4 to 10 was followed using compounds 3g [6-chloro-8-(2-fluoro-4-(trifluoromethyl)phenyl)-2,3-dimethylpyrimido[5,4-d]pyrimidin-4 (3H)-one and 2-(1-cyclopropyl-1H-pyrazol-4-yl)morpholine [synthesized in example-2, step-10] as starting materials to afford 6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one as yellow solid in 59.8% yield.

### Example-21

### Example-21A: Enantiomer 1

### (R)-8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one

### Example-21B: Enantiomer 2

### (S)-8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one

### Step-1—Preparation of 8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one

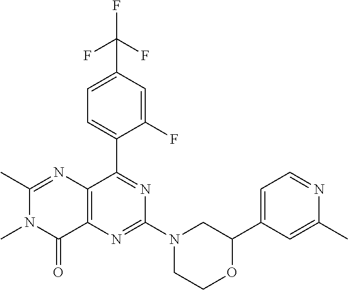

General procedure of Step-2, example-4 to 10 was followed using compounds 3g [6-chloro-8-(2-fluoro-4-(trifluoromethyl)phenyl)-2,3-dimethylpyrimido[5,4-d]pyrimidin-4 (3H)-one and 2-(2-methylpyridin-4-yl)morpholine [synthesized in example-3, step-2] as starting materials to afford 8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one as yellow solid in 56.6% yield.

### Example-22

### Example-22A: Enantiomer 1

### (R)-2,3-dimethyl-6-(2-(2-methylpyridin-4-yl)morpholino)-8-(6-(trifluoromethyl)pyridin-3-yl)pyrimido[5,4-d]pyrimidin-4 (3H)-one

### Example-22B: Enantiomer 2

### (S)-2,3-dimethyl-6-(2-(2-methylpyridin-4-yl)morpholino)-8-(6-(trifluoromethyl)pyridin-3-yl)pyrimido[5,4-d]pyrimidin-4 (3H)-one

### Step-1—Preparation of 2,3-dimethyl-6-(2-(2-methylpyridin-4-yl)morpholino)-8-(6-(trifluoromethyl)pyridin-3-yl)pyrimido[5,4-d]pyrimidin-4 (3H)-one

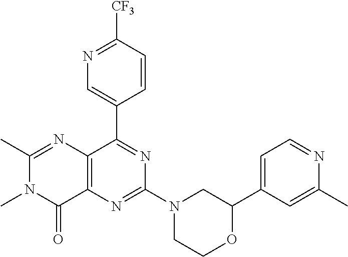

General procedure of Step-2, example-4 to 10 was followed using compounds 3a [6-chloro-2,3-dimethyl-8-(6-(trifluoromethyl)pyridin-3-yl)pyrimido[5,4-d]pyrimidin-4 (3H)-one] and 2-(2-methylpyridin-4-yl)morpholine [synthesized in example-3, step-2] as starting materials to afford 2,3-dimethyl-6-(2-(2-methylpyridin-4-yl)morpholino)-8-(6-(trifluoromethyl)pyridin-3-yl)pyrimido[5,4-d]pyrimidin-4 (3H)-one as yellow solid in 53.6% yield.

Chiral separation details of Example 19A-19B to 28A-28B are captured in following

| | | | Chiral |
|---|---|---|---|
| | | Starting | separation |
| Compound No./Structure | Name | material | specification |
| Ex. 19A (Peak 1) 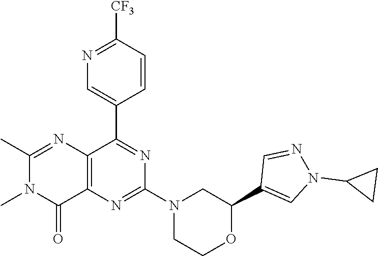 [SMILES: Cc1nc2c(-c3ccc(C(F)(F)F)nc3)nc(N3CCO[C@H](c4cnn(C5CC5)c4)C3)nc2c(=O)n1C] | (R)-6-(2-(1-cyclopropyl- 1H-pyrazol-4- yl)morpholino)-2,3- dimethyl-8-(6- (trifluoromethyl)pyridin- 3-yl)pyrimido[5,4- d]pyrimidin-4(3H)-one | 6-(2-(1- cyclopropyl- 1H- pyrazol-4- yl)morpholi- no)-2,3- dimethyl-8- (6- (trifluorometh- yl)pyridin- 3- yl)pyrimido[5,4- d]pyrimidin- 4(3H)-one | Similar specifications as mentioned in step-6 of example-1. Peak 1 Absolute stereochemistry unknown |
| Ex. 19B (Peak 2) 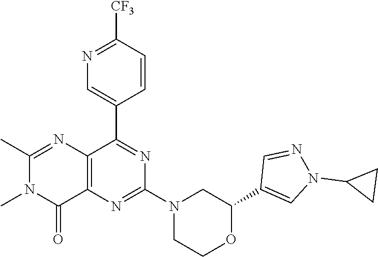 [SMILES: Cc1nc2c(-c3ccc(C(F)(F)F)nc3)nc(N3CCO[C@@H](c4cnn(C5CC5)c4)C3)nc2c(=O)n1C] | (S)-6-(2-(1-cyclopropyl- 1H-pyrazol-4- yl)morpholino)-2,3- dimethyl-8-(6- (trifluoromethyl)pyridin- 3-yl)pyrimido[5,4- d]pyrimidin-4(3H)-one | | Peak 2 Absolute stereochemistry unknown |
| Ex. 20A (Peak 1) 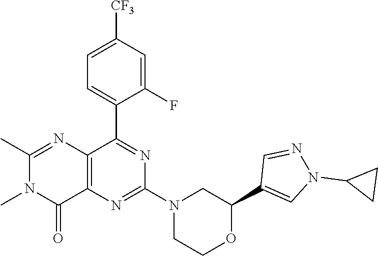 [SMILES: Cc1nc2c(-c3ccc(C(F)(F)F)cc3F)nc(N3CCO[C@H](c4cnn(C5CC5)c4)C3)nc2c(=O)n1C] | (R)-6-(2-(1-cyclopropyl- 1H-pyrazol-4- yl)morpholino)-8-(2- fluoro-4- (trifluoromethyl)phenyl)- 2,3- dimethylpyrimido[5,4- d]pyrimidin-4(3H)-one | 6-(2-(1- cyclopropyl- 1H- pyrazol-4- yl)morpholi- no)-8-(2- fluoro-4- (trifluorometh- yl)phenyl)- 2,3- dimethylpyri- mido[5,4- d]pyrimidin- 4(3H)-one | Similar specifications as mentioned in step-6 of example-1. Peak 1 Absolute stereochemistry unknown |
| Ex. 20B (Peak 2) 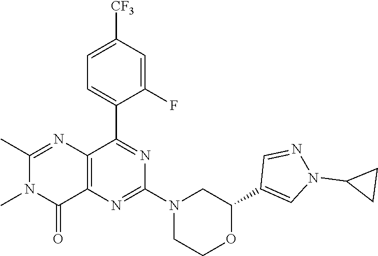 [SMILES: Cc1nc2c(-c3ccc(C(F)(F)F)cc3F)nc(N3CCO[C@@H](c4cnn(C5CC5)c4)C3)nc2c(=O)n1C] | (S)-6-(2-(1-cyclopropyl- 1H-pyrazol-4- yl)morpholino)-8-(2- fluoro-4- (trifluoromethyl)phenyl)- 2,3- dimethylpyrimido[5,4- d]pyrimidin-4(3H)-one | | Peak 2 Absolute stereochemistry unknown |
| Ex. 21A (Peak 1) 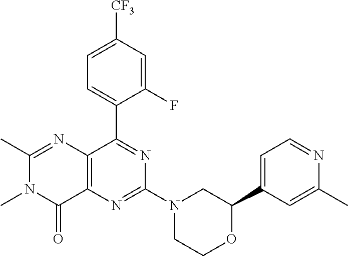 | (R)-8-(2-fluoro-4- (trifluoromethyl)phenyl)- 2,3-dimethyl-6-(2-(2- methylpyridin-4- yl)morpholino)pyrimido[5,4- d]pyrimidin-4(3H)-one | 8-(2-fluoro- 4- (trifluorometh- yl)phenyl)- 2,3- dimethyl-6- (2-(2- methylpyridin- 4- yl)morpholi- no)pyrimido [5,4- d]pyrimidin- 4(3H)-one | Agilent 1200 series instrument. Column name: CHIRALART CELLULOSE SC (250 × 20 mm) 5u. Operating at ambient temperature and flow rate is 18.0 mL/min. Mobile phase was mixture of 75% Hexane, 12.5% DCM and 12.5% ETOH, held this isocratic mixture run up to 25 min |
| | | | with wavelength |
| | | | of 296 nm. |
| | | | Peak 1 |
| | | | Absolute |
| | | | stereochemistry |
| | | | unknown |
| Ex. 21B (Peak 2) 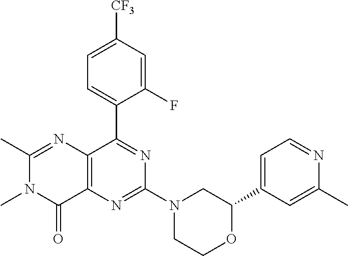 | (S)-8-(2-fluoro-4- (trifluoromethyl)phenyl)- 2,3-dimethyl-6-(2-(2- methylpyridin-4- yl)morpholino)pyrimido[5,4- d]pyrimidin-4(3H)-one | | Peak 2 Absolute stereochemistry unknown |
| Ex. 22A (Peak 1) 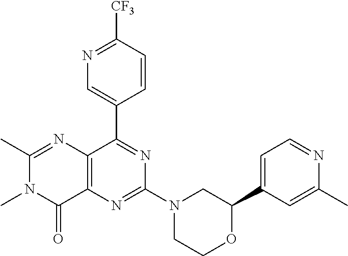 | (R)-2,3-dimethyl-6-(2-(2- methylpyridin-4- yl)morpholino)-8-(6- (trifluoromethyl)pyridin- 3-yl)pyrimido[5,4- d]pyrimidin-4(3H)-one | 2,3- dimethyl-6- (2-(2- methylpyridin- 4- yl)morpholi- no)-8-(6- (trifluorometh- yl)pyridin- 3- yl)pyrimido[5,4- d]pyrimidin- 4(3H)-one | Similar specifications as mentioned in step-6 of example-1. Peak 1 Absolute stereochemistry unknown |
| Ex. 22B (Peak 2) 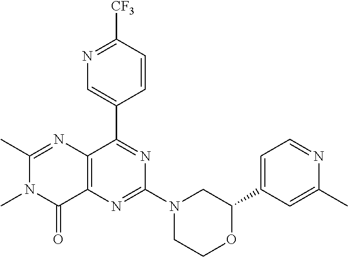 | (S)-2,3-dimethyl-6-(2-(2- methylpyridin-4- yl)morpholino)-8-(6- (trifluoromethyl)pyridin- 3-yl)pyrimido[5,4- d]pyrimidin-4(3H)-one | | Peak 2 Absolute stereochemistry unknown |
| Ex. 23A (Peak 1) 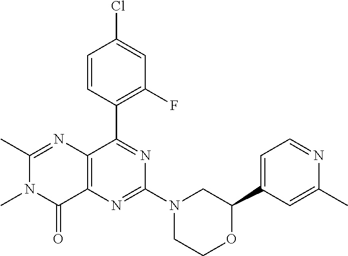 | (R)-8-(4-chloro-2- fluorophenyl)-2,3- dimethyl-6-(2-(2- methylpyridin-4- yl)morpholino)pyrimido[5,4- d]pyrimidin-4(3H)-one | Example-3 | Similar specifications as mentioned in step-6 of example-1. Peak 1 Absolute stereochemistry unknown |
| Ex. 23B (Peak 2) 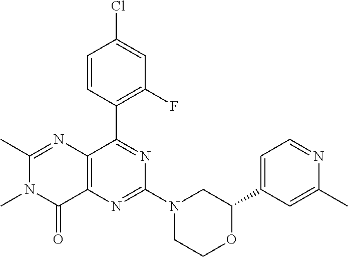 | (S)-8-(4-chloro-2- fluorophenyl)-2,3- dimethyl-6-(2-(2- methylpyridin-4- yl)morpholino)pyrimido[5,4- d]pyrimidin-4(3H)-one | | Peak 2 Absolute stereochemistry unknown |
| Ex. 24A (Peak 1) 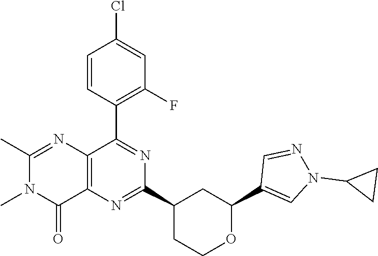 [SMILES: Cc1nc2c(-c3ccc(Cl)cc3F)nc([C@@H]3CCO[C@H](c4cnn(C5CC5)c4)C3)nc2c(=O)n1C] | 8-(4-chloro-2- fluorophenyl)-6- ((2S,4R)-2-(1- cyclopropyl-1H-pyrazol- 4-yl)tetrahydro-2H- pyran-4-yl)-2,3- dimethylpyrimido[5,4- d]pyrimidin-4(3H)-one | Example- 14 | CHIRALPAK IG (250 × 21 mm) 5u. Operating at ambient temperature and flow rate is 21.0 mL/min. Mobile phase was mixture of 50% Hexane, 25% DCM, 25% Ethyl alcohol, held this isocratic mixture run up to 23 min with wavelength of 276 nm. Peak 1 Absolute |
| | | | stereochemistry |
| | | | unknown |
| Ex. 24B (Peak 2) 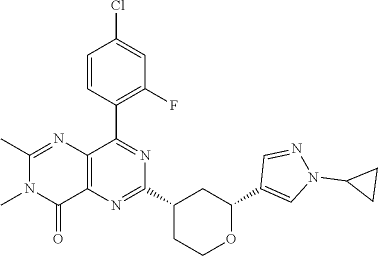 [SMILES: Cc1nc2c(-c3ccc(Cl)cc3F)nc([C@H]3CCO[C@@H](c4cnn(C5CC5)c4)C3)nc2c(=O)n1C] | 8-(4-chloro-2- fluorophenyl)-6- ((2R,4S)-2-(1- cyclopropyl-1H-pyrazol- 4-yl)tetrahydro-2H- pyran-4-yl)-2,3- dimethylpyrimido[5,4- d]pyrimidin-4(3H)-one | | Peak 2 Absolute stereochemistry unknown |
| Ex. 25A (Peak 1) 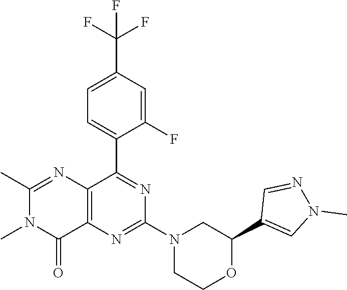 | (R)-8-(2-fluoro-4- (trifluoromethyl)phenyl)- 2,3-dimethyl-6-(2-(1- methyl-1H-pyrazol-4- yl)morpholino)pyrimido[5,4- d]pyrimidin-4(3H)-one | Example- 10 | Similar specifications as mentioned in step-6 of example-1 Peak 1 Absolute stereochemistry unknown |
| Ex. 25B (Peak 2) 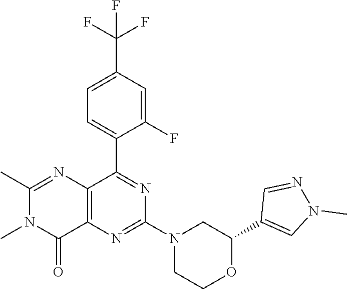 | (S)-8-(2-fluoro-4- (trifluoromethyl)phenyl)- 2,3-dimethyl-6-(2-(1- methyl-1H-pyrazol-4- yl)morpholino)pyrimido[5,4- d]pyrimidin-4(3H)-one | | Peak 2 Absolute stereochemistry unknown |
| Ex. 26A (Peak 1) 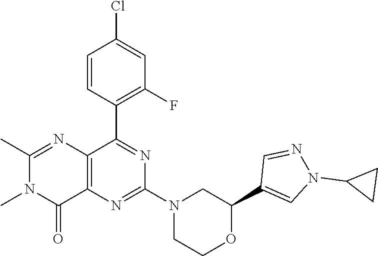 [SMILES: Cc1nc2c(-c3ccc(Cl)cc3F)nc(N3CCO[C@H](c4cnn(C5CC5)c4)C3)nc2c(=O)n1C] | (R)-8-(4-chloro-2- fluorophenyl)-6-(2-(1- cyclopropyl-1H-pyrazol- 4-yl)morpholino)-2,3- dimethylpyrimido[5,4- d]pyrimidin-4(3H)-one | Example-2 | Similar specifications as mentioned in step-6 of example-1. Peak 1 Absolute stereochemistry unknown |
| Ex. 26B (Peak 2) 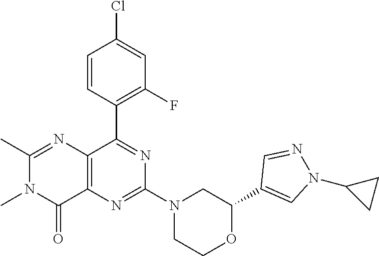 [SMILES: Cc1nc2c(-c3ccc(Cl)cc3F)nc(N3CCO[C@@H](c4cnn(C5CC5)c4)C3)nc2c(=O)n1C] | (S)-8-(4-chloro-2- fluorophenyl)-6-(2-(1- cyclopropyl-1H-pyrazol- 4-yl)morpholino)-2,3- dimethylpyrimido[5,4- d]pyrimidin-4(3H)-one | | Peak 2 Absolute stereochemistry unknown |
| Ex. 27A (Peak 1) 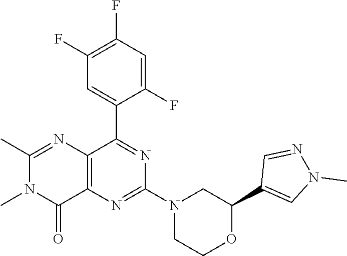 | (R)-2,3-dimethyl-6-(2-(1- methyl-1H-pyrazol-4- yl)morpholino)-8-(2,4,5- trifluorophenyl)pyrimido[5,4- d]pyrimidin-4(3H)- one | Example-7 | Similar specifications as mentioned in step-6 of example-1. Absolute stereochemistry unknown |
| Ex. 27B (Peak 2) 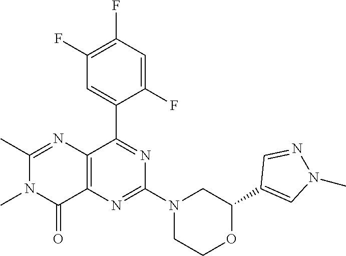 | (S)-2,3-dimethyl-6-(2-(1- methyl-1H-pyrazol-4- yl)morpholino)-8-(2,4,5- trifluorophenyl)pyrimido[5,4- d]pyrimidin-4(3H)- one | | Absolute stereochemistry unknown |
| Ex. 28A (Peak 1) 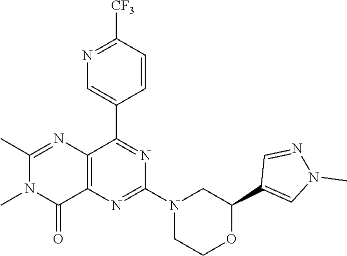 | (R)-2,3-dimethyl-6-(2-(1- methyl-1H-pyrazol-4- yl)morpholino)-8-(6- (trifluoromethyl)pyridin- 3-yl)pyrimido[5,4- d]pyrimidin-4(3H)-one | Example-4 | Similar specifications as mentioned in step-6 of example-1. Absolute stereochemistry unknown |
| Ex. 28B (Peak 2) 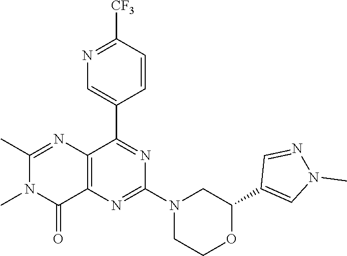 | (S)-2,3-dimethyl-6-(2-(1- methyl-1H-pyrazol-4- yl)morpholino)-8-(6- (trifluoromethyl)pyridin- 3-yl)pyrimido[5,4- d]pyrimidin-4(3H)-one | | Absolute stereochemistry unknown |

Following table describes analytical data analysis and yield information of examples 19A-191B to examples 28A-28B:

| Compound | | | Analytical | | Yield |
|---|---|---|---|---|---|
| No. | HNMR | LCMS | chiral HPLC | Description | (%) |
| Ex. 19A | 1H NMR (400 MHz, DMSO-d6) δ | Condition | Chiralpak IC | Yellow | 45.4 |
| (Peak 1) | 9.60 (s, 1H), 8.90 (d, J = 8.0 Hz, | K: Rt = | (4.6 × 250 | solid | |
| | 1H), 8.09 (d, J = 8.4 Hz, 1H), 7.86 | 2.83 min. | mm), 5 μ Mobile | | |
| | (s, 1H), 7.48 (s, 1H), 4.70 (d, J = | m/z 513.4 | Phase: | | |
| | 12.4 Hz, 1H), 4.61 (d, J = 13.2 | [M + H]⁺. | Hexane/DCM/ | | |
| | Hz, 1H), 4.51 (d, J = 10.0 Hz, 1H), | | EtOH/IPAmine: | | |
| | 4.03 (d, J = 10.8 Hz, 1H), 3.74- | | 60/20/20/0.1 | | |
| | 3.62 (m, 2H), 3.53 (s, 3H), 3.27- | | Flow Rate: 1.0 | | |
| | 3.14 (m, 2H), 2.54 (s, 3H), 1.06- | | ml/min | | |
| | 1.00 (m, 2H), 0.99-0.89 (m, 2H). | | Solubility: | | |
| Ex. 19B | 1H NMR (400 MHz, DMSO-d6) δ | Condition | MeOH Rt = | Yellow | 41.1 |
| (Peak 2) | 9.60 (s, 1H), 8.90 (d, J = 8.4 Hz, | K: Rt = | 6.11 min for | solid | |
| | 1H), 8.09 (d, J = 8.4 Hz, 1H), 7.86 | 2.83 min. | Peak 1 and Rt = | | |
| | (s, 1H), 7.48 (s, 1H), 4.70 (d, J = | m/z 513.4 | 7.04 min for | | |
| | 12.8 Hz, 1H), 4.61 (d, J = 13.2 | [M + H]⁺. | Peak 2. | | |
| | Hz, 1H), 4.51 (d, J = 10.4 Hz, 1H), | | | | |
| | 4.03 (d, J = 10.4 Hz, 1H), 3.75- | | | | |
| | 3.62 (m, 2H), 3.54 (s, 3H), 3.28- | | | | |
| | 3.14 (m, 2H), 2.54 (s, 3H), 1.09- | | | | |
| | 0.88 (m, 4H). | | | | |
| Ex. 20A | 1H NMR (400 MHz, DMSO-d6) δ | Condition | Chiralpak IC | Yellow | 44.7 |
| (Peak 1) | 7.90-7.81 (m, 3H), 7.75 (d, J = | K: Rt = | (4.6 × 250 | solid | |
| | 8.3 Hz, 1H), 7.45 (s, 1H), 4.61 (d, | 3.35 min. | mm), 5 μ Mobile | | |
| | J = 13.0 Hz, 1H), 4.56-4.45 (m, | m/z 530.4 | Phase: | | |
| | 2H), 4.00 (d, J = 9.2 Hz, 1H), 3.71- | [M + H]⁺. | Hexane/DCM/ | | |
| | 3.63 (m, 2H), 3.51 (s, 3H), 3.22- | | EtOH/IPAmine: | | |
| | 3.12 (m, 2H), 2.43 (s, 3H), 1.07- | | 50/25/25/0.1 | | |
| | 0.96 (m, 2H), 0.97-0.90 (m, 2H). | | Flow Rate: 1.0 | | |
| Ex. 20B | 1H NMR (400 MHz, DMSO-d6) δ | Condition | ml/min | Yellow | 42.8 |
| (Peak 2) | 7.90-7.81 (m, 3H), 7.75 (d, J = | K: Rt = | Solubility: | solid | |
| | 7.3 Hz, 1H), 7.46 (s, 1H), 4.61 (d, | 3.35 min. | MeOH Rt = | | |
| | J = 13.6 Hz, 1H), 4.50 (t, J = 8.2 | m/z 530.4 | 6.73 min for | | |
| | Hz, 2H), 4.00 (d, J = 12.0 Hz, 1H), | [M + H]⁺. | Peak 1 and Rt = | | |
| | 3.73-3.63 (m, 2H), 3.51 (s, 3H), | | 7.88 min for | | |
| | 3.25-3.12 (m, 2H), 2.43 (s, 3H), | | Peak 2. | | |
| | 1.06-0.96 (m, 2H), 0.98-0.90 (m, 2H). | | | | |
| Ex. 21A | 1H NMR (400 MHz, DMSO-d6) δ | Condition | Chiral Pak IC | Yellow | 66.4 |
| (Peak 1) | 8.45 (d, J = 4.8 Hz, 1H), 7.94- | K: Rt = | (4.6 × 250 | solid | |
| | 7.83 (m, 2H), 7.76 (d, J = 8.0 Hz, | 2.52 min. | mm), 5 μ Mobile | | |
| | 1H), 7.31 (s, 1H), 7.23 (d, J = 4.8 | m/z 515.4 | Phase: | | |
| | Hz, 1H), 4.70 (d, J = 13.0 Hz, 1H), | [M + H]⁺. | Hexane/DCM/ | | |
| | 4.60 (t, J = 11.1 Hz, 2H), 4.14 (d, | | EtOH/IPAmine: | | |
| | J = 10.6 Hz, 1H), 3.74 (t, J = 11.4 | | 50/25/25/0.1 | | |
| | Hz, 1H), 3.52 (s, 3H), 3.22 (t, J = | | Flow Rate: 1.0 | | |
| | 11.8 Hz, 1H), 2.99 (t, J = 11.4 Hz, | | ml/min | | |
| | 1H), 2.50 (s, 3H), 2.43 (s, 3H). | | Solubility: | | |
| Ex. 21B | 1H NMR (400 MHz, DMSO-d6) δ 8.47 | Condition | MeOH Rt = | Yellow | 61.3 |
| (Peak 2) | (d, J = 5.0 Hz, 1H), 7.88 (d, J = | K: Rt = | 6.07 min for | solid | |
| | 8.4 Hz, 2H), 7.76 (d, J = 8.1 Hz, | 2.52 min. | Peak 1 and Rt = | | |
| | 1H), 7.36 (s, 1H), 7.28 (d, J = 3.9 | m/z 515.4 | 8.34 min for | | |
| | Hz, 1H), 4.71 (d, J = 12.8 Hz, 1H), | [M + H]⁺. | Peak 2. | | |
| | 4.61 (t, J = 17.7 Hz, 2H), 4.15 (d, | | | | |
| | J = 9.4 Hz, 1H), 3.75 (t, J = 11.8 | | | | |
| | Hz, 1H), 3.52 (s, 3H), 3.25-3.16 | | | | |
| | (m, 1H), 2.99 (t, J = 10.9 Hz, 1H), | | | | |
| | 2.50 (s, 3H), 2.44 (s, 3H). | | | | |
| Ex. 22A | 1H NMR (400 MHz, DMSO-d6) δ | Condition | Chiral Pak IC | Yellow | 59.9 |
| (Peak 1) | 9.60 (s, 1H), 8.92 (dd, J = 1.2, 8.0 | K: Rt = | (4.6 × 250 | solid | |
| | Hz, 1H), 8.47 (d, J = 5.0 Hz, 1H), | 2.61 min. | mm), 5 μ Mobile | | |
| | 8.11 (d, J = 8.2 Hz, 1H), 7.34 (s, | m/z 498.4 | Phase: | | |
| | 1H), 7.27 (d, J = 5.0 Hz, 1H), 4.79 | [M + H]⁺. | Hexane/DCM/ | | |
| | (d, J = 12.8 Hz, 1H), 4.73-4.58 | | EtOH/IPAmine: | | |
| | (m, 2H), 4.17 (dd, J = 2.3, 11.4 | | 50/25/25/0.1 | | |
| | Hz, 1H), 3.82-3.70 (m, 1H), 3.54 | | Flow Rate: 1.0 | | |
| | (s, 3H), 3.30-3.19 (m, 1H), 3.02 | | ml/min | | |
| | (t, J = 10.6, 13.0 Hz, 1H), 2.56 (s, | | Solubility: | | |
| | 3H), 2.43 (s, 3H). | | MeOH Rt = | | |
| Ex. 22B | 1H NMR (400 MHz, DMSO-d6) δ | Condition | 8.28 min for | Yellow | 55.3 |
| (Peak 2) | 9.60 (s, 1H), 8.92 (d, J = 8.0 Hz, | K: Rt = 2.61 | Peak 1 and Rt = | solid | |
| | 1H), 8.47 (d, J = 5.0 Hz, 1H), 8.11 | min. m/z | 10.44 min for | | |
| | (d, J = 8.2 Hz, 1H), 7.34 (s, 1H), | 498.4 | Peak 2. | | |
| | 7.27 (d, J = 4.8 Hz, 1H), 4.79 (d, | [M + H]⁺. | | | |
| | J = 12.4 Hz, 1H), 4.73-4.58 (m, | | | | |
| | 2H), 4.17 (d, J = 9.4 Hz, 1H), 3.82- | | | | |
| | 3.71 (m, 1H), 3.54 (s, 3H), 3.28- | | | | |
| | 3.19 (m, 1H), 3.07-2.96 (m, | | | | |
| | 1H), 2.56 (s, 3H), 2.43 (s, 3H). | | | | |
| Ex. 23A | 1H NMR (400 MHz, DMSO-d6) δ | Condition | Chiral Pak IC | Off- | 35.9 |
| (Peak 1) | 8.45 (d, J = 5.1 Hz, 1H), 7.68 (t, | K: Rt = 2.53 | (4.6 × 250 | white | |
| | J = 7.9 Hz, 1H), 7.62 (dd, J = 2.0, | min. m/z | mm), 5 μ Mobile | solid | |
| | 9.8 Hz, 1H), 7.46 (d, J = 8.1 Hz, | 481.2 | Phase: | | |
| | 1H), 7.31 (s, 1H), 7.23 (d, J = 4.7 | [M + H]⁺. | Hexane/DCM/ | | |
| | Hz, 1H), 4.70 (d, J = 12.9 Hz, 1H), | | EtOH/IPAmine: | | |
| | 4.65-4.55 (m, 2H), 4.14 (d, J = | | 50/25/25/0.1 | | |
| | 10.5 Hz, 1H), 3.79-3.69 (m, 1H), | | Flow Rate: 1.0 | | |
| | 3.51 (s, 3H), 3.21 (t, J = 12.4 Hz, | | ml/min | | |
| | 1H), 3.03-2.92 (m, 1H), 2.50 (s, | | Solubility: | | |
| | 3H), 2.44 (s, 3H). | | MeOH Rt = | | |
| Ex. 23B | 1H NMR (400 MHz, DMSO-d6) δ | Condition K: | 7.25 min for | Beige | 31.4 |
| (Peak 2) | 8.45 (d, J = 4.9 Hz, 1H), 7.68 (t, | Rt = 2.53 | Peak 1 and Rt = | solid | |
| | J = 8.0 Hz, 1H), 7.65-7.59 (m, | min. m/z | 10.98 min for | | |
| | 1H), 7.46 (dd, J = 2.2, 8.5 Hz, | 481.2 | Peak 2. | | |
| | 1H), 7.31 (s, 1H), 7.23 (d, J = 5.4 | [M + H]⁺. | | | |
| | Hz, 1H), 4.70 (d, J = 11.2 Hz, 1H), | | | | |
| | 4.63-4.55 (m, 2H), 4.14 (d, J = | | | | |
| | 10.7 Hz, 1H), 3.74 (t, J = 11.4 Hz, | | | | |
| | 1H), 3.51 (s, 3H), 3.27-3.15 (m, | | | | |
| | 1H), 3.03-2.93 (m, 1H), 2.50 (s, | | | | |
| | 3H), 2.44 (s, 3H). | | | | |
| Ex. 24A | 1H NMR (400 MHz, DMSO-d6) δ | Condition | Chiral Pak IC | White | 22.3 |
| (Peak 1) | 7.73-7.67 (m, 2H), 7.64 (dd, J = | K: Rt = | (4.6 × 250 | solid | |
| | 1.8, 10.0 Hz, 1H), 7.49 (dd, J = | 3.14 min. | mm), 5 μ Mobile | | |
| | 1.8, 8.3 Hz, 1H), 7.36 (s, 1H), | m/z 495.4 | Phase: | | |
| | 4.49 (d, J = 11.0 Hz, 1H), 4.08 | [M + H]⁺. | Hexane/DCM/ | | |
| | (dd, J = 3.9, 11.3 Hz, 1H), 3.74- | | EtOH/IPAmine: | | |
| | 3.59 (m, 2H), 3.55 (s, 3H), 3.47- | | 50/25/25/0.1 | | |
| | 3.35 (m, 1H), 2.52 (s, 3H), 2.22 | | Flow Rate: 1.0 | | |
| | (d, J = 13.0 Hz, 1H), 2.02-1.78 | | ml/min | | |
| | (m, 3H), 1.04-0.83 (m, 4H). | | Solubility: | | |
| Ex. 24B | 1H NMR (400 MHz, DMSO-d6) δ | Condition | MeOH Rt = | White | 18.4 |
| (Peak 2) | 7.73-7.67 (m, 2H), 7.64 (dd, J = | K: Rt = | 8.22 min for | solid | |
| | 1.8, 10.0 Hz, 1H), 7.49 (dd, J = | 3.14 min. | Peak 1 and Rt = | | |
| | 1.8, 8.2 Hz, 1H), 7.36 (s, 1H), | m/z 495.4 | 9.11 min for | | |
| | 4.49 (dd, J = 2.0, 11.3 Hz, 1H), | [M + H]⁺. | Peak 2. | | |
| | 4.08 (dd, J = 3.4, 11.0 Hz, 1H), | | | | |
| | 3.74-3.59 (m, 2H), 3.56 (s, 3H), | | | | |
| | 3.47-3.35 (m, 1H), 2.53 (s, 3H), | | | | |
| | 2.26-2.17 (m, 1H), 2.02-1.78 | | | | |
| | (m, 3H), 1.04-0.85 (m, 4H). | | | | |
| Ex. 25A | 1H NMR (400 MHz, DMSO) δ | Condition | Chiral Pak IC | Yellow | 49.2 |
| (Peak 1) | 7.91-7.82 (m, 2H), 7.75 (d, J = | K: Rt = | (4.6 × 250 | solid | |
| | 7.3 Hz, 2H), 7.45 (s, 1H), 4.66- | 2.90 min. | mm), 5 μ Mobile | | |
| | 4.58 (m, 1H), 4.55-4.47 (m, 2H), | m/z 504.4 | Phase: | | |
| | 4.01 (d, J = 11.5 Hz, 1H), 3.81 (s, | [M + H]⁺. | Hexane/DCM/ | | |
| | 3H), 3.72-3.61 (m, 1H), 3.51 (s, | | EtOH/IPAmine: | | |
| | 3H), 3.25-3.11 (m, 2H), 2.43 (s, 3H). | | 50/25/25/0.1 | | |
| Ex. 25B | 1H NMR (400 MHz, DMSO) δ | Condition | Flow Rate: 1.0 | Yellow | 47.4 |
| (Peak 2) | 7.90-7.82 (m, 2H), 7.75 (d, J = | K: Rt = | ml/min | solid | |
| | 7.0 Hz, 2H), 7.45 (s, 1H), 4.62 (d, | 2.90 min. | Solubility: | | |
| | J = 13.3 Hz, 1H), 4.52 (d, J = 10.5 | m/z 504.4 | MeOH Rt = | | |
| | Hz, 2H), 4.00 (d, J = 11.4 Hz, 1H), | [M + H]⁺. | 6.95 min for | | |
| | 3.81 (s, 3H), 3.66 (t, J = 11.2 Hz, | | Peak 1 and Rt = | | |
| | 1H), 3.51 (s, 3H), 3.26-3.11 (m, | | 7.78 min for | | |
| | 2H), 2.43 (s, 3H). | | Peak 2. | | |
| Ex. 26A | 1H NMR (400 MHz, DMSO-d6) δ | Condition | Chiral Pak IC | White | 36.8 |
| (Peak 1) | 7.84 (s, 1H), 7.71-7.57 (m, 2H), | K: Rt = | (4.6 × 250 | solid | |
| | 7.49-7.41 (m, 2H), 4.61 (d, J = | 2.98 min. | mm), 5 μ Mobile | | |
| | 13.1 Hz, 1H), 4.56-4.44 (m, 2H), | m/z 496.3 | Phase: | | |
| | 3.99 (d, J = 10.9 Hz, 1H), 3.74- | [M + H]⁺. | Hexane/DCM/ | | |
| | 3.59 (m, 2H), 3.50 (s, 3H), 3.24- | | EtOH/IPAmine: | | |
| | 3.11 (m, 2H), 2.43 (s, 3H), 1.10- | | 50/25/25/0.1 | | |
| | 0.88 (m, 4H). | | Flow Rate: 1.0 | | |
| Ex. 26B | 1H NMR (400 MHz, DMSO) δ | Condition | ml/min | Yellow | 33.2 |
| (Peak 2) | 7.83 (s, 1H), 7.66 (t, J = 7.9 Hz, | K: Rt = | Solubility: | solid | |
| | 1H), 7.61 (d, J = 9.7 Hz, 1H), 7.49- | 2.98 min. | MeOH Rt = | | |
| | 7.42 (m, 2H), 4.61 (d, J = 13.4 | m/z 496.3 | 7.95 min for | | |
| | Hz, 1H), 4.55-4.45 (m, 2H), 4.00 | [M + H]⁺. | Peak 1 and Rt = | | |
| | (d, J = 11.2 Hz, 1H), 3.74-3.59 | | 9.63 min for | | |
| | (m, 2H), 3.50 (s, 3H), 3.23-3.11 | | Peak 2. | | |
| | (m, 2H), 2.43 (s, 3H), 1.06-0.88 | | | | |
| | (m, 4H). | | | | |
| Ex. 27A | 1H NMR (400 MHz, DMSO-d6) δ | Condition K: | Chiral Pak IC | Yellow | 38.9 |
| (Peak 1) | 7.92-7.60 (m, 3H), 7.45 (s, 1H), | Rt = 2.79 | (4.6 × 250 | solid | |
| | 4.62 (d, J = 13.2 Hz, 1H), 4.51 (d, | min. m/z | mm), 5 μ Mobile | | |
| | J = 10.3 Hz, 2H), 4.00 (d, J = 11.8 | 472.3 | Phase: | | |
| | Hz, 1H), 3.81 (s, 3H), 3.66 (t, J = | [M + H]⁺. | Hexane/DCM/ | | |
| | 11.3 Hz, 1H), 3.51 (s, 3H), 3.25- | | EtOH/IPAmine: | | |
| | 3.10 (m, 2H), 2.44 (s, 3H). | | 50/25/25/0.1 | | |
| Ex. 27B | 1H NMR (400 MHz, DMSO-d6) δ | Condition K: | Flow Rate: 1.0 | Yellow | 33.6 |
| (Peak 2) | 7.85-7.66 (m, 3H), 7.45 (s, 1H), | Rt = 2.79 | ml/min | solid | |
| | 4.62 (d, J = 13.0 Hz, 1H), 4.51 (d, | min. m/z | Solubility: | | |
| | J = 10.3 Hz, 2H), 4.01 (d, J = 11.3 | 472.3 | MeOH Rt = | | |
| | Hz, 1H), 3.81 (s, 3H), 3.71-3.61 | [M + H]⁺. | 7.88 min for | | |
| | (m, 1H), 3.51 (s, 3H), 3.25-3.10 | | Peak 1 and Rt = | | |
| | (m, 2H), 2.44 (s, 3H). | | 8.94 min for | | |
| | | | Peak 2. | | |
| Ex. 28A | 1H NMR (400 MHz, DMSO-d6) δ | Condition | Chiral Pak IC | Yellow | 46.4 |
| (Peak 1) | 9.60 (s, 1H), 8.90 (d, J = 8.2 Hz, | K: Rt = | (4.6 × 250 | solid | |
| | 1H), 8.10 (d, J = 8.2 Hz, 1H), 7.77 | 3.11 min. | mm), 5 μ Mobile | | |
| | (s, 1H), 7.48 (s, 1H), 4.71 (d, J = | m/z 487.4 | Phase: | | |
| | 13.1 Hz, 1H), 4.61 (d, J = 12.9 | [M + H]⁺. | Hexane/DCM/ | | |
| | Hz, 1H), 4.53 (d, J = 10.3 Hz, 1H), | | EtOH/IPAmine: | | |
| | 4.04 (d, J = 10.5 Hz, 1H), 3.82 (s, | | 50/25/25/0.1 | | |
| | 3H), 3.68 (t, J = 11.6 Hz, 1H), | | Flow Rate: 1.0 | | |
| | 3.54 (s, 3H), 3.25-3.13 (m, 2H), | | ml/min | | |
| | 2.55 (s, 3H). | | Solubility: | | |
| Ex. 28B | 1H NMR (400 MHz, DMSO-d6) δ | Condition | MeOH Rt = | Yellow | 44.5 |
| (Peak 2) | 9.60 (s, 1H), 8.90 (d, J = 7.9 Hz, | K: Rt = | 9.37 min for | solid | |
| | 1H), 8.10 (d, J = 8.2 Hz, 1H), 7.77 | 3.11 min. | Peak 1 and Rt = | | |
| | (s, 1H), 7.48 (s, 1H), 4.71 (d, J = | m/z 487.4 | 11.04 min for | | |
| | 12.8 Hz, 1H), 4.61 (d, J = 13.2 | [M + H]⁺. | Peak 2. | | |
| | Hz, 1H), 4.53 (dd, J = 2.8, 10.3 | | | | |
| | Hz, 1H), 4.04 (d, J = 10.7 Hz, 1H), | | | | |
| | 3.82 (s, 3H), 3.68 (t, J = 11.5 Hz, | | | | |
| | 1H), 3.54 (s, 3H), 3.29-3.13 (m, | | | | |
| | 2H), 2.55 (s, 3H). | | | | |

### Synthesis of Tert-Butyl 3-(1-Methyl-1H-Pyrazol-4-Yl)Piperazine-1-Carboxylate Used for Synthesis of Examples 29 to 34

### Step-1—Preparation of 2-(1-methyl-1H-pyrazol-4-yl)pyrazine

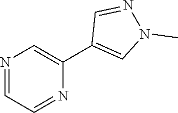

To a stirred solution of 2-chloropyrazine (2.6 g, 22.81 mmol) and 1-methyl-4-(4,4,5,5-tetramethyl-1,3,2-dioxaborolan-2-yl)-1H-pyrazole (5.2 g, 25.09 mmol) in 1,2-dimethoxyethane (100 ml) and water (20 mL) was added sodium carbonate (7.2 g, 1.22 mmol) and degassed with argon. PdCl2(dppf)·DCM (0.931 g, 1.14 mmol) was added under inert atmosphere. Resulting mixture was heated at 80° C. for 16 h. Reaction mixture was filtered through a short pad of celite and washed with ethyl acetate. Combined organic part was washed with water, brine, dried over anhydrous sodium sulphate, filtered and concentrated under reduced pressure. Crude mass was purified by column chromatography (80% ethyl acetate-hexane) to afford 2-(1-methyl-1H-pyrazol-4-yl)pyrazine (3.1 g, 86.11% yield) as brown solid.

1H NMR (400 MHz, CDCl3) δ 8.74 (s, 1H), 8.46 (s, 1H), 8.34 (d, J=2.3 Hz, 1H), 7.99 (s, 1H), 7.95 (s, 1H), 3.96 (s, 3H)

LCMS Condition E: Rt=1.95 min, m/z 161.2 [M+H]⁺.

### Step-2—Preparation of 2-(1-methyl-1H-pyrazol-4-yl)piperazine

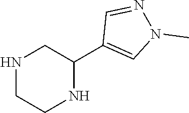

To a degassed solution of 2-(1-methyl-1H-pyrazol-4-yl)pyrazine (800 mg, 4.97 mmol) in ethanol (15 mL) and acetic acid (3 mL), PtO2 (563.9 mg, 2.48 mmol) was added. Resulting mixture was hydrogenated under H2 balloon pressure for 16 h at RT. Reaction mixture was filtered through a short pad of celite and washed with ethanol. Combined filtrate was concentrated under reduced pressure to afford 2-(1-methyl-1H-pyrazol-4-yl)piperazine (420 mg, 50.8% yield) as white solid.

1H NMR (400 MHz, DMSO-d6) δ 7.54 (s, 1H), 7.31 (s, 1H), 3.76 (s, 3H), 3.60-3.57 (m, 1H), 2.89-2.40 (m, 8H)

LCMS Condition F: Rt=0.85 min, m/z 167.3 [M+H]⁺.

### Step-3—Preparation of tert-butyl 3-(1-methyl-1H-pyrazol-4-yl)piperazine-1-carboxylate

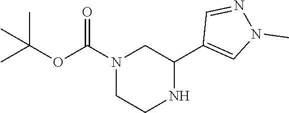 [SMILES: Cn1cc(C2CN(C(=O)OC(C)(C)C)CCN2)cn1]

To a stirred solution of 2-(1-methyl-1H-pyrazol-4-yl)piperazine (600 mg, 3.61 mmol) in Dioxane (12 mL) and water (6 mL) at 0° C. was added triethyl amine (1 ml, 7.23 mmol) followed by Boc-anhydride (394 mg, 1.81 mmol) and DMAP (26 mg, 0.22 mmol). Resulting mixture was stirred at RT for 16 h. Reaction mass was diluted with cold water and extracted with 10% methanol-dichloromethane. Combined organic part was washed with water, brine, dried over anhydrous sodium sulphate, filtered and concentrated under reduced pressure. Crude mass was purified by column chromatography (5% MeOH in DCM) to afford tert-butyl 3-(1-methyl-1H-pyrazol-4-yl)piperazine-1-carboxylate (380 mg, 39.4% yield) as white solid.

1H NMR (400 MHz, CDCl3) δ 7.44 (s, 1H), 7.33 (s, 1H), 4.04-4.01 (m, 1H), 3.85 (s, 3H), 3.72-3.70 (m, 1H), 3.07-2.79 (m, 4H), 2.20 (br s, 2H) 1.45 (s, 9H)

LCMS Condition E: Rt=2.94 min, m/z 267.1 [M+H]⁺.

### General Conditions for Prepare of N-Alkylated Intermediates Using tert-butyl 3-(1-methyl-1H-pyrazol-4-yl)piperazine-1-carboxylate to Synthesize Examples 29 to 34

### Condition-A

To a stirred solution of tert-butyl 3-(1-methyl-1H-pyrazol-4-yl)piperazine-1-carboxylate (140 mg, 0.53 mmol) in ethanol (3 mL) and water (1 mL), paraformaldehyde (63 mg, 2.10 mmol) was added. Resulting mixture and heated at 60° C. for 1 h. It was then cooled to RT, sodium cyanoborohydride (83 mg, 1.32 mmol) and acetic acid (0.2 mL) was added. Resulting mixture was again heated at 60° C. for 16 h. Reaction mixture was quenched with saturated NaHCO₃ solution and concentrated under reduced pressure. It was diluted with cold water and extracted with 10% methanol-dichloromethane. Combined organic part was washed with water, brine, dried over anhydrous sodium sulphate, filtered and concentrated under reduced pressure. Crude mass was purified by column chromatography (10% MeOH in DCM) to afford desired product.

Condition-B: To a stirred solution of tert-butyl 3-(1-methyl-1H-pyrazol-4-yl)piperazine-1-carboxylate (0.45 mmol) in MeCN (2 ml) were added corresponding halide (0.41 mmol), K₂CO₃ (0.41 mmol) and KI (0.41 mmol). Resulting mixture was heated at 80° C. for 16 h. After completion, reaction mixture was diluted with cold water and extracted with 5% MeOH-DCM. Combine organic layer was dried over sodium sulphate, filtered and concentrated under reduced pressure. Crude product was purified by column chromatography (5-10% MeOH in DCM) to afford desired products.

### Condition-C

To a stirred solution of tert-butyl 3-(1-methyl-1H-pyrazol-4-yl)piperazine-1-carboxylate (100 mg, 0.38 mmol) in THF (2 ml) were added 2,2,2-trifluoroethyl trifluoromethanesulfonate (0.054 mL, 0.38 mmol), and DIPEA (0.06 mL, 0.38 mmol). Resulting mixture was heated at 70° C. for 16 h. After completion, reaction mixture was diluted with cold water and extracted with 5% MeOH-DCM. Combine organic layer was dried over sodium sulphate, filtered and concentrated under reduced pressure. Crude product was purified by column chromatography (5-10% MeOH in DCM) to afford desired product.

| Cpd# | Alkylating | Condi- | | | Yield |
|---|---|---|---|---|---|
| Structure | SM | tion | HNMR | LCMS | (%) |
| 6a 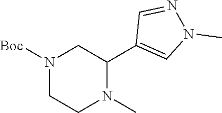 [SMILES: CN1CCN(C(=O)OC(C)(C)C)CC1c1cnn(C)c1] | Parafor- maldehyde | A | 1H NMR (400 MHz, DMSO D6) δ 7.59 (s, 1H), 7.32 (s, 1H), 3.79 (s, 3H), 3.78-3.65 (m, 2H), 2.99-2.76 (m, 3H), 2.51-2.49 (m, 2H), 1.97 (s, 3H) 1.38 (s, 9H) | Condition A: Rt = 2.24 min, m/z 280.9 [M + H]+. | 47.5 |
| 6b 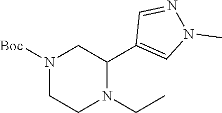 [SMILES: CCN1CCN(C(=O)OC(C)(C)C)CC1c1cnn(C)c1] | Ethyl bromide | B | 1H NMR (400 MHz, DMSO D6) δ 7.59 (s, 1H), 7.32 (s, 1H), 3.79 (s, 3H), 3.75-3.59 (m, 2H), 3.17-3.15 (m, 1H), 3.05-3.0 (m, 1H), 2.83-2.80 (m, 1H), 2.49-2.33 (m, 2H), 2.06- 2.00 (m, 2H), 1.38 (s, 9H), 0.87 (t, J = 7.0 Hz, 3H) | Condition F: Rt = 3.45 min, m/z 295.0 [M + H]⁺. | 56.5 |
| 6c 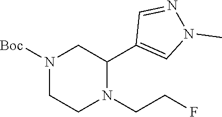 [SMILES: Cn1cc(C2CN(C(=O)OC(C)(C)C)CCN2CCF)cn1] | 1- bromo- 2- fluoroeth- ane | B | 1H NMR (400 MHz, DMSO D6) δ 7.60 (s, 1H), 7.33 (s, 1H), 4.49-4.33 (m, 3H), 3.79 (s, 3H), 3.73-3.62 (m, 2H), 3.09-3.06 (m, 1H), 2.90-2.89 (m, 1H), 2.36-2.32 (m, 2H), 2.25-2.19 (m, 2H), 1.39 (s, 9H) | Condition F: Rt = 3.32 min, m/z 313.1 [M + H]⁺. | 28.4 |
| 6d 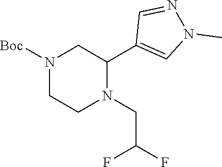 [SMILES: Cn1cc(C2CN(C(=O)OC(C)(C)C)CCN2CC(F)F)cn1] | 1,1- difluoro- 2- iodoethane | B[KI was not used] | 1H NMR (400 MHz, DMSO D6) δ 7.65 (s, 1H), 7.36 (s, 1H), 5.97 (t, J = 57.6 Hz, 1H), 3.80 (s, 3H), 3.73-3.67 (m, 2H), 3.38-3.36 (m, 1H), 3.10- 3.09 (m, 1H), 2.93-2.65 (m, 2H), 2.51-2.49 (m, 2H), 2.37- 2.28 (m, 1H), 1.38 (s, 9H) | Condition A: Rt = 2.94 min, m/z 331.0 [M + H]⁺. | 61.2 |
| 6e 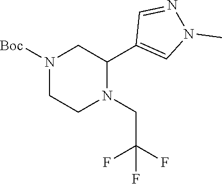 [SMILES: Cn1cc(C2CN(C(=O)OC(C)(C)C)CCN2CC(F)(F)F)cn1] | 2,2,2- trifluoro- ethyl trifluoro- methane- sulfonate | C | 1H NMR (400 MHz, DMSO D6) δ 7.64 (s, 1H), 7.36 (s, 1H), 3.80 (s, 3H), 3.67-3.60 (m, 2H), 3.50-3.48 (m, 1H), 3.15-2.90 (m, 5H), 2.50-2.49 (m, 1H), 1.38 (s, 9H) | Condition F: Rt = 3.34 min, m/z 348.9 [M + H]⁺. | 61.1 |
| 6f 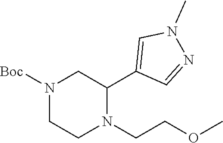 [SMILES: COCCN1CCN(C(=O)OC(C)(C)C)CC1c1cnn(C)c1] | 1- bromo- 2- methoxy- ethane | B | 1H NMR (400 MHz, DMSO D6) δ 7.59 (s, 1H), 7.33 (s, 1H), 3.79 (s, 3H), 3.71-3.68 (m, 3H), 3.29-3.28 (m, 2H), 3.22-3.21 (m, 3H), 3.15 (s, 3H) 2.90-2.87 (m, 2H), 2.61-2.57 (m, 1H), 1.37 (s, 9H) | Condition D: Rt = 2.90 min, m/z 324.9 [M + H]+. | 57.5 |

N-boc deprotection of all intermediate alkylated compounds (6a to 6f) was done following usual condition mentioned in step-2, example-3 followed by amination reaction using boc-deprotected-6a to 6f amines and 6-chloro-8-(4-chloro-2-fluorophenyl)-2,3-dimethylpyrimido[5,4-d]pyrimidin-4 (3H)-one [synthesized in step-4, example-1] following condition mentioned in general procedure of Step-2, example-4 to 10.

Examples 29 to 34 were isolated as yellow solid.

HNMR, LCMS and yield percentage information of examples 29 to 34 are provided in the table below:

| | | | | | |
|---|---|---|---|---|---|
| Compound | | | | | Yield |
| No. | Structure | Name | HNMR | LCMS | (%) |

| | | | | |
|---|---|---|---|---|
| Ex.29 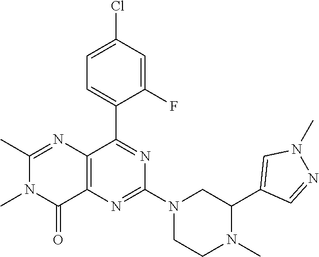 | 8-(4-chloro-2- fluorophenyl)- 2,3-dimethyl-6- (4-methyl-3-(1- methyl-1H- pyrazol-4- yl)piperazin-1- yl)pyrimido[5,4- d]pyrimidin- 4(3H)-one | 1H NMR (400 MHz, DMSO-d6) δ 7.69- 7.55 (m, 3H), 7.44 (d, J = 8.3 Hz, 1H), 7.38 (s, 1H), 4.58 (d, J = 12.6 Hz, 1H), 4.48 (d, J = 11.2 Hz, 1H), 3.80 (s, 3H), 3.50 (s, 3H), 3.32- 3.19 (m, 1H), 3.15- 2.91 (m, 3H), 2.42 (s, 3H), 2.24-2.13 (m, 1H), 2.03 (s, 3H). | Condition K: Rt = 2.36 min, m/z 483.3 [M + H]+. | 45.4 |
| Ex. 30 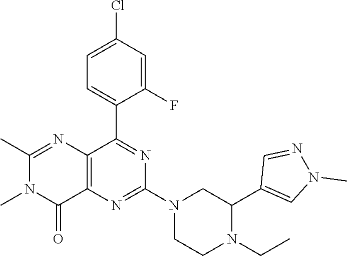 | 8-(4-chloro-2- fluorophenyl)-6- (4-ethyl-3-(1- methyl-1H- pyrazol-4- yl)piperazin-1- yl)-2,3- dimethylpyrimido [5,4- d]pyrimidin- 4(3H)-one | 1H NMR (400 MHz, DMSO-d6) δ 7.65- 7.58 (m, 3H), 7.44 (d, J = 8.5 Hz, 1H), 7.38 (s, 1H), 4.65- 4.26 (m, 2H), 3.80 (s, 3H), 3.50 (s, 3H), 3.43-3.33 (m, 1H), 3.20 (t, J = 10.0 Hz, 1H), 3.01 (d, J = 11.0 Hz, 1H), 2.69- 2.52 (m, 2H), 2.42 (s, 3H), 2.31-1.97 (m, 2H), 0.91 (t, J = 5.6 Hz, 3H). | Condition k: Rt = 2.51 min, m/z 497.3 [M + H]+. | 38.9 |
| Ex. 31 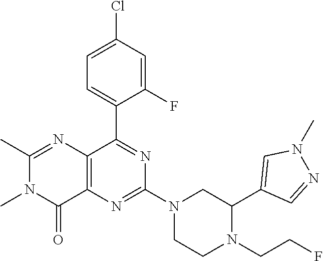 [SMILES: Cc1nc2c(-c3ccc(Cl)cc3F)nc(N3CCN(CCF)C(c4cnn(C)c4)C3)nc2c(=O)n1C] | 8-(4-chloro-2- fluorophenyl)-6- (4-(2- fluoroethyl)-3-(1- methyl-1H- pyrazol-4- yl)piperazin-1- yl)-2,3- dimethylpyrimido [5,4- d]pyrimidin- 4(3H)-one | 1H NMR (400 MHz, DMSO) δ 7.75- 7.54 (m, 3H), 7.44 (d, J = 8.0 Hz, 1H), 7.38 (s, 1H), 4.66- 4.27 (m, 4H), 3.80 (s, 3H), 3.50 (s, 3H), 3.46-3.37 (m, 1H), 3.29-3.18 (m, 1H), 3.16-3.02 (m, 1H), 2.93-2.72 (m, 1H), 2.64-2.27 (m, 6H). | Condition K: Rt = 2.46 min, m/z 515.3 [M + H]+. | 55.2 |
| Ex.32 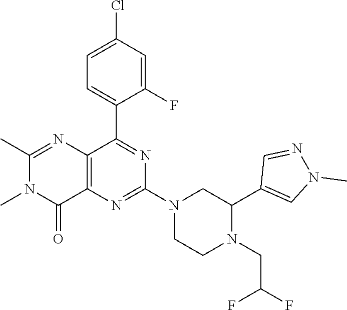 | 8-(4-chloro-2- fluorophenyl)-6- (4-(2,2- difluoroethyl)-3- (1-methyl-1H- pyrazol-4- yl)piperazin-1- yl)-2,3- dimethylpyrimido [5,4- d]pyrimidin- 4(3H)-one | 1H NMR (400 MHz, DMSO-d6) δ 7.69 (s, 1H), 7.67-7.56 (m, 2H), 7.48-7.39 (m, 2H), 6.02 (t, J = 54.3 Hz, 1H), 4.42 (t, J = 13.8 Hz, 2H), 3.82 (s, 3H), 3.57-3.50 (m, 1H), 3.50 (s, 3H), 3.45-3.34 (m, 1H), 3.11 (d, J = 11.8 Hz, 1H), 2.92- 2.76 (m, 2H), 2.68- 2.50 (m, 2H), 2.43 (s, 3H). | Condition K: Rt = 3.14 min, m/z 533.4 [M + H]+. | 32.1 |
| Ex. 33 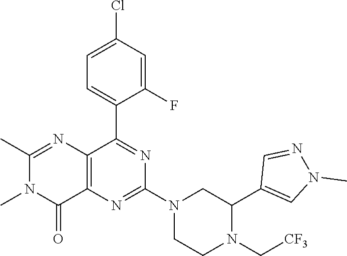 | 8-(4-chloro-2- fluorophenyl)- 2,3-dimethyl-6- (3-(1-methyl-1H- pyrazol-4-yl)-4- (2,2,2- trifluoroethyl)piper- azin-1- yl)pyrimido[5,4- d]pyrimidin- 4(3H)-one | 1H NMR (400 MHz, DMSO-d6) δ 7.69 (s, 1H), 7.68-7.55 (m, 2H), 7.44 (d, J = 8.0 Hz, 1H), 7.40 (s, 1H), 4.38 (t, J = 12.8 Hz, 2H), 3.81 (s, 3H), 3.65 (dd, J = 3.1, 9.1 Hz, 1H), 3.56-3.43 (m, 4H), 3.41 (dd, J = 9.2, 13.0 Hz, 1H), 3.24- 2.92 (m, 3H), 2.74- 2.58 (m, 1H), 2.43 (s, 3H). | Condition K: Rt = 3.30 min, m/z 551.5 [M + H]+. | 62.3 |
| Ex.34 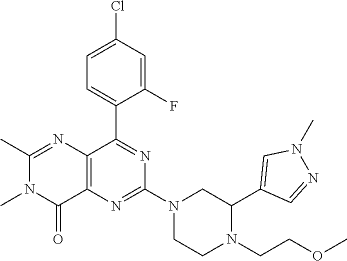 | 8-(4-chloro-2- fluorophenyl)-6- (4-(2- methoxyethyl)- 3-(1-methyl-1H- pyrazol-4- yl)piperazin-1- yl)-2,3- dimethylpyrimido [5,4- d]pyrimidin- 4(3H)-one | 1H NMR (400 MHz, DMSO-d6) δ 7.68- 7.56 (m, 3H), 7.44 (d, J = 8.2 Hz, 1H), 7.38 (s, 1H), 4.50- 4.37 (m, 2H), 3.80 (s, 3H), 3.50 (s, 3H), 3.40-3.32 (m, 3H), 3.23-3.21 (m, 1H), 3.18 (s, 3H), 3.08 (d, J = 12.2 Hz, 1H), 2.75-2.61 (m, 1H), 2.42 (s, 3H), 2.40- 2.28 (m, 2H), 2.24- 2.16 (m, 1H). | Condition K: Rt = 2.43 min, m/z 527.3 [M + H]+. | 46.1 |

### Example 35

### 8-(4-chloro-2-fluorophenyl)-2,3-dimethyl-6-(2-(2-methylpyrimidin-5-yl)morpholino)pyrimido[5,4-d]pyrimidin-4 (3H)-one

### Example 36

### 8-(4-chloro-2-fluorophenyl)-2,3-dimethyl-6-(2-(1-methyl-1H-pyrazol-3-yl)morpholino)pyrimido[5,4-d]pyrimidin-4 (3H)-one

Example 35 and 36 were synthesized following similar protocol used for synthesis of example 2.

For example-35, benzyl deprotection was done using following procedure:

### Preparation of 2-(2-methylpyrimidin-5-yl)morpholine

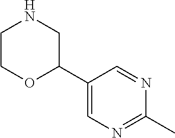

To a degassed solution of 4-benzyl-2-(2-methylpyrimidin-5-yl)morpholine (150.0 mg, 0.56 mmol) in methanol (5 mL), ammonium formate (175.651 mg, 2.78 mmol) and palladium hydroxide on carbon (50% wet, 100 mg) were added. The resulting mixture was heated under argon atmosphere at 80° C. for 2 h. The reaction mixture was filtered through a short pad of celite and washed with methanol. The combined filtrate was concentrated under reduced pressure. The crude material was purified by triturating with ether to afford 2-(2-methylpyrimidin-5-yl)morpholine (80 mg, 80.8% yield) as sticky liquid.

### Example 37

### 8-(4-chloro-2-fluorophenyl)-2,3-dimethyl-6-((2R,4S)-2-(2-methylpyrimidin-5-yl)tetrahydro-2H-pyran-4-yl)pyrimido[5,4-d]pyrimidin-4 (3H)-one [cis-racemate]

### Example 38

### Example 38A

### 2,3-dimethyl-6-((2S,4R)-2-(1-methyl-1H-pyrazol-4-yl)tetrahydro-2H-pyran-4-yl)-8-(2,4,5-trifluorophenyl)pyrimido[5,4-d]pyrimidin-4 (3H)-one

### Example 38B

### 2,3-dimethyl-6-((2R,4S)-2-(1-methyl-1H-pyrazol-4-yl)tetrahydro-2H-pyran-4-yl)-8-(2,4,5-trifluorophenyl)pyrimido[5,4-d]pyrimidin-4 (3H)-one

Example 37, examples 38A and 38B were synthesized following similar protocol as used for synthesis of example 14.

Details of Example 35 to example 38B are captured in following table:

| | | | | |
|---|---|---|---|---|
| | | | Intermediate | Chiral |
| Compound | | | compounds/SMs | separation |
| No. | Structure | Name | used | specification |

| | | | |
|---|---|---|---|
| Ex. 35 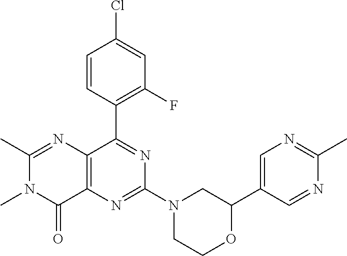 | 8-(4-chloro-2- fluorophenyl)- 2,3-dimethyl-6- (2-(2- methylpyrimidin- 5- yl)morpholino)py- rimido[5,4- d]pyrimidin- 4(3H)-one | 2-(2-methylpyrimidin- 5-yl)morpholine 6-chloro-8-(4-chloro-2- fluorophenyl)-2,3- dimethylpyrimido[5,4- d]pyrimidin-4(3H)-one | |
| Ex. 36 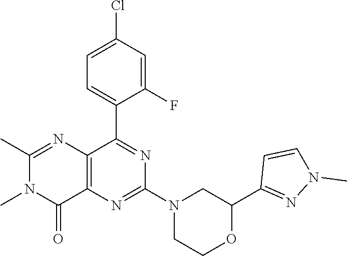 | 8-(4-chloro-2- fluorophenyl)- 2,3-dimethyl-6- (2-(1-methyl-1H- pyrazol-3- yl)morpholino)py- rimido[5,4- d]pyrimidin- 4(3H)-one | 2-(1-methyl-1H- pyrazol-3- yl)morpholine 6-chloro-8-(4-chloro-2- fluorophenyl)-2,3- dimethylpyrimido[5,4- d]pyrimidin-4(3H)-one | |
| Ex. 37 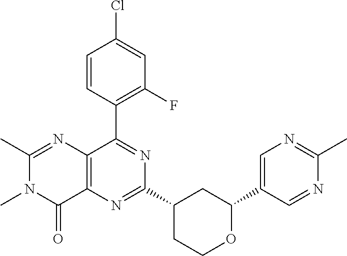 | 8-(4-chloro-2- fluorophenyl)- 2,3-dimethyl-6- ((2R,4S)-2-(2- methylpyrimidin- 5-yl)tetrahydro- 2H-pyran-4- yl)pyrimido[5,4- d]pyrimidin- 4(3H)-one | 8-(4-chloro-2- fluorophenyl)-2,3- dimethyl-6-(6-(2- methylpyrimidin-5-yl)- 3,6-dihydro-2H-pyran- 4-yl)pyrimido[5,4- d]pyrimidin-4(3H)-one 6-chloro-8-(4-chloro-2- fluorophenyl)-2,3- dimethylpyrimido[5,4- d]pyrimidin-4(3H)-one | Absolute stereochemistry |
| Ex. 38A (Peak 1) 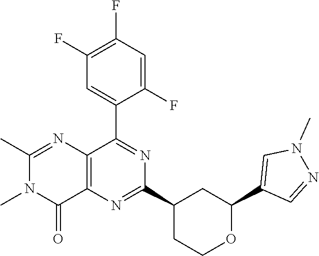 [SMILES: Cc1nc2c(-c3cc(F)c(F)cc3F)nc([C@@H]3CCO[C@H](c4cnn(C)c4)C3)nc2c(=O)n1C] | 2,3-dimethyl-6- ((2S,4R)-2-(1- methyl-1H- pyrazol-4- yl)tetrahydro- 2H-pyran-4-yl)- 8-(2,4,5- trifluorophenyl)py- rimido[5,4- d]pyrimidin- 4(3H)-one | 6-chloro-2,3-diemthyl- 8-(2,4,5- trifluorophenyl)pyrimido [5,4-d]pyrimidin- 4(3H)-one (3d) | Similar specifications as mentioned in step-6 of example-1. Absolute stereochemistry unknown |
| Ex. 38B (Peak 2) 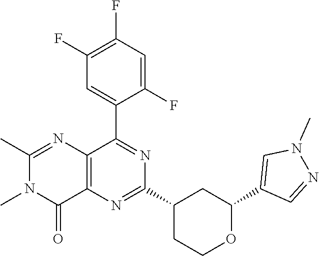 [SMILES: Cc1nc2c(-c3cc(F)c(F)cc3F)nc([C@H]3CCO[C@@H](c4cnn(C)c4)C3)nc2c(=O)n1C] | 2,3-dimethyl-6- ((2R,4S)-2-(1- methyl-1H- pyrazol-4- yl)tetrahydro- 2H-pyran-4-yl)- 8-(2,4,5- trifluorophenyl)py- rimido[5,4- d]pyrimidin- 4(3H)-one | 2,3-dimethyl-6- ((2R,4S)-2-(1-methyl- 1H-pyrazol-4- yl)tetrahydro-2H- pyran-4-yl)-8-(2,4,5- trifluorophenyl)pyrimido [5,4-d]pyrimidin- 4(3H)-one (cis racemate) | Absolute stereochemistry unknown |

Following table describes analytical data analysis and yield information of examples 35A-351B to examples 38A-38B:

| Compound | | | Analytical | | Yield |
|---|---|---|---|---|---|
| No. | HNMR | LCMS | chiral HPLC | Description | (%) |
| Ex. 35 | 1H NMR (400 MHz, DMSO-d6) δ | Condition | | Yellow | 23.4 |
| | 8.77 (s, 2H), 7.68 (t, J = 7.9 Hz, | K: Rt = | | solid | |
| | 1H), 7.61 (d, J = 9.8 Hz, 1H), 7.45 | 2.90 min. | | | |
| | (d, J = 8.2 Hz, 1H), 4.67 (dd, J = | m/z 482.3 | | | |
| | 6.1, 10.0 Hz, 2H), 4.58 (d, J = 13.2 | [M + H]⁺. | | | |
| | Hz, 1H), 4.13 (d, J = 12.0 Hz, 1H), | | | | |
| | 3.80-3.67 (m, 1H), 3.51 (s, 3H), | | | | |
| | 3.27-3.12 (m, 2H), 2.63 (s, 3H), | | | | |
| | 2.44 (s, 3H). | | | | |
| Ex. 36 | 1H NMR (400 MHz, DMSO-d6) δ | Condition | | Yellow | 36.5 |
| | 7.71-7.57 (m, 3H), 7.46 (dd, J = | K: Rt = | | solid | |
| | 2.0, 8.4 Hz, 1H), 6.26 (d, J = 2.0 | 3.23 min. | | | |
| | Hz, 1H), 4.77 (d, J = 12.4 Hz, 1H), | m/z 470.3 | | | |
| | 4.53 (t, J = 8.0 Hz, 2H), 4.04 (d, J = | [M + H]⁺. | | | |
| | 12.0 Hz, 1H), 3.82 (s, 3H), 3.75- | | | | |
| | 3.64 (m, 1H), 3.51 (s, 3H), 3.18 (t, | | | | |
| | J = 12.8 Hz, 2H), 2.43 (s, 3H). | | | | |
| Ex. 37 | 1H NMR (400 MHz, DMSO-d6) δ | Condition | | White | 21.2 |
| | 8.69 (s, 2H), 7.74-7.59 (m, 2H), | P: Rt = | | solid | |
| | 7.48 (dd, J = 1.9, 8.3 Hz, 1H), 4.67 | 2.52 min. | | | |
| | (d, J = 11.1 Hz, 1H), 4.23 (dd, J = | m/z 481.1 | | | |
| | 4.0, 11.2 Hz, 1H), 3.77 (t, J = 11.8 | [M + H]⁺. | | | |
| | Hz, 1H), 3.55 (s, 4H), 2.60 (s, 3H), | | | | |
| | 2.53 (s, 3H), 2.32 (d, J = 11.3 Hz, | | | | |
| | 1H), 2.12-1.89 (m, 2H), 1.80 (q, | | | | |
| | J = 12.1 Hz, 1H). | | | | |
| Ex. 38A | 1H NMR (400 MHz, DMSO-d6) δ | Condition | Chiral Pak IC | White | 58.4 |
| (Peak 1) | 7.88-7.69 (m, 2H), 7.63 (s, 1H), | Q: Rt = | (4.6 × 250 | solid | |
| | 7.36 (s, 1H), 4.50 (d, J = 11.1 Hz, | 7.78 min. | mm), 5 μ Mobile | | |
| | 1H), 4.09 (dd, J = 3.7, 11.4 Hz, 1H), | m/z 470.8 | Phase: | | |
| | 3.78 (s, 3H), 3.69 (t, J = 10.7 Hz, | [M + H]⁺. | Hexane/DCM/ | | |
| | 1H), 3.56 (s, 3H), 3.48-3.36 (m, | | EtOH/IPAmine: | | |
| | 1H), 2.53 (s, 3H), 2.21 (d, J = 12.8 | | 60/20/20/0.1 | | |
| | Hz, 1H), 2.02-1.79 (m, 3H). | | Flow Rate: 1.0 | | |
| Ex. 38B | 1H NMR (400 MHz, DMSO) δ 7.87- | Condition | ml/min | White | 52.3 |
| (Peak 2) | 7.70 (m, 2H), 7.63 (s, 1H), 7.36 | Q: Rt = | Solubility: | solid | |
| | (s, 1H), 4.50 (d, J = 11.1 Hz, 1H), | 7.78 min. | MeOH Rt = | | |
| | 4.09 (dd, J = 10.8, 3.3 Hz, 1H), | m/z 470.8 | 11.74 min for | | |
| | 3.78 (s, 3H), 3.74-3.63 (m, 1H), | [M + H]⁺. | Peak 1 and Rt = | | |
| | 3.56 (s, 3H), 3.47-3.37 (m, 1H), | | 14.10 min for | | |
| | 2.53 (s, 3H), 2.22 (d, J = 13.0 Hz, | | Peak 2. | | |
| | 1H), 2.05-1.78 (m, 3H). | | | | |

### Example 39

### 6-(2-(I H-pyrazol-4-yl)morpholino)-8-(4-chloro-2-fluorophenyl)-2,3-dimethylpyrimido[5,4-d]pyrimidin-4 (3H)-one

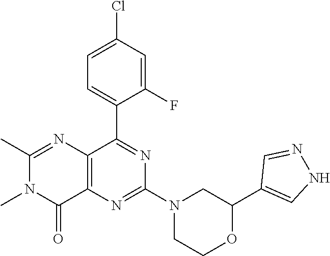 [SMILES: Cc1nc2c(-c3ccc(Cl)cc3F)nc(N3CCOC(c4cn[nH]c4)C3)nc2c(=O)n1C]

6-(2-(1H-pyrazol-4-yl)morpholino)-8-(4-chloro-2-fluorophenyl)-2,3-dimethylpyrimido[5,4-d]pyrimidin-4 (3H)-one [example-39] was synthesized as yellow solid in 43.4% yield using 6-chloro-8-(4-chloro-2-fluorophenyl)-2,3-dimethylpyrimido[5,4-d]pyrimidin-4 (3H)-one [procedure in example-1, step-4] and 2-(1H-pyrazol-4-yl)morpholine [procedure in example-2, step-7] following usual condition mentioned in general procedure of Step-2, example-4 to 10

1H NMR (400 MHz, DMSO D6) δ 12.94-12.72 (m, 1H), 7.79 (s, 1H), 7.67 (t, J=8.0 Hz, 1H), 7.60 (d, J=9.0 Hz, 1H), 7.53 (s, 1H), 7.45 (d, J=8.0 Hz, 1H), 4.62 (d, J=13.3 Hz, 1H), 4.53 (t, J=14.8 Hz, 2H), 4.01 (d, J=10.9 Hz, 1H), 3.66 (t, J=11.4 Hz, 1H), 3.50 (s, 3H), 3.20 (t, J=13.0 Hz, 2H), 2.43 (s, 3H).

LCMS Condition K: Rt=2.81 min, m/z 456.25 [M+H]⁺.

### Example 40

### 8-(4-chloro-2-fluorophenyl)-2,3-dimethyl-6-(2-(1-(oxetan-3-yl)-1H-pyrazol-4-yl)morpholino)pyrimido[5,4-d]pyrimidin-4 (3H)-one

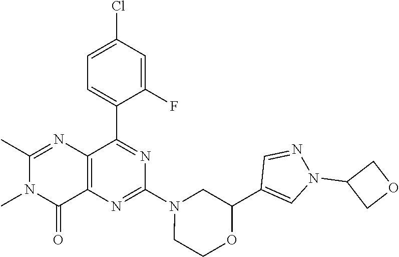 [SMILES: Cc1nc2c(-c3ccc(Cl)cc3F)nc(N3CCOC(c4cnn(C5COC5)c4)C3)nc2c(=O)n1C]

To a stirred solution of 6-(2-(1H-pyrazol-4-yl)morpholino)-8-(4-chloro-2-fluorophenyl)-2,3-dimethylpyrimido[5,4-d]pyrimidin-4 (3H)-one [example-39](100 mg, 0.219 mmol) in dry DMF (1 mL) at 0° C. was added sodium hydride (6.85 mg, 0.285 mmol). Resulting suspension was stirred for 10 min and a solution of 3-iodooxetane (0.021 mL, 0.241 mmol) in DMF (0.5 mL) was added dropwise. Resulting mixture was heated at 65° C. for 3 h. After completion, reaction mixture was cooled to ambient temperature, diluted with cold water and extracted with ethyl acetate. Combined organic layer was washed with brine, dried over anhydrous sodium sulphate, filtered and concentrated under reduced pressure. Crude mass was purified by reverse phase preparative HPLC to afford 8-(4-chloro-2-fluorophenyl)-2,3-dimethyl-6-(2-(1-(oxetan-3-yl)-1H-pyrazol-4-yl)morpholino)pyrimido[5,4-d]pyrimidin-4 (3H)-one (33 mg, 29.2% yield) as a yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 60% A and 40% B in 3 min, then to 20% A and 80% B in 20 min., then to 5% A and 95% B in 21 min., held this composition up to 22 min. for column washing, then returned to initial composition in 23 min. and held till 25 min.

1H NMR (400 MHz, DMSO-d6) δ 7.96 (s, 1H), 7.71-7.57 (m, 3H), 7.46 (d, J=8.0 Hz, 1H), 5.56 (t, J=6.9 Hz, 1H), 4.94-4.85 (m, 4H), 4.64 (d, J=15.0 Hz, 1H), 4.58-4.50 (m, 2H), 4.01 (d, J=18.2 Hz, 1H), 3.67 (t, J=11.1 Hz, 1H), 3.50 (s, 3H), 3.23-3.12 (m, 2H), 2.43 (s, 3H).

LCMS Condition K: Rt=3.16 min, m/z 512.3 [M+H]⁺.

### Example 41

### 6-(2-(1-(azetidin-3-yl)-1H-pyrazol-4-yl)morpholino)-8-(4-chloro-2-fluorophenyl)-2,3-dimethylpyrimido[5,4-d]pyrimidin-4 (3H)-one hydrochloride salt

### Step-1—Preparation of tert-butyl 3-(4-(4-(4-(4-chloro-2-fluorophenyl)-6,7-dimethyl-8-oxo-7,8-dihydropyrimido[5,4-d]pyrimidin-2-yl)morpholin-2-yl)-1H-pyrazol-1-yl)azetidine-1-carboxylate

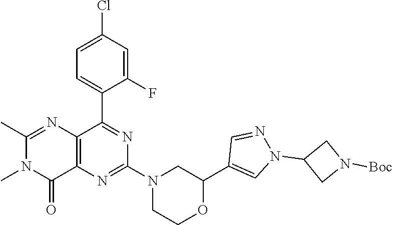 [SMILES: Cc1nc2c(-c3ccc(Cl)cc3F)nc(N3CCOC(c4cnn(C5CN(C(=O)OC(C)(C)C)C5)c4)C3)nc2c(=O)n1C]

Procedure of step-1, example-16, was followed using example-39 and tert-butyl 3-iodoazetidine-1-carboxylate [commercial] as starting materials to afford tert-butyl 3-(4-(4-(4-(4-chloro-2-fluorophenyl)-6,7-dimethyl-8-oxo-7,8-dihydropyrimido[5,4-d]pyrimidin-2-yl)morpholin-2-yl)-1H-pyrazol-1-yl)azetidine-1-carboxylate as off white solid in 43.5% yield.

1H NMR (400 MHz, DMSO-d6) δ 7.95 (s, 1H), 7.68-7.59 (m, 3H), 7.46-7.44 (m, 1H), 5.18 (m, 1H), 4.64-4.51 (m, 3H), 4.26-4.24 (m, 2H), 4.01-3.99 (m, 3H), 3.68-3.66 (m, 1H), 3.51 (s, 3H), 3.19-3.13 (m, 2H), 2.43 (s, 3H), 1.41 (s, 9H).

LCMS Condition D: Rt=3.56 min, m/z 611.2 [M+H]⁺.

### Step-2—Preparation of 6-(2-(1-(azetidin-3-yl)-1H-pyrazol-4-yl)morpholino)-8-(4-chloro-2-fluorophenyl)-2,3-dimethylpyrimido[5,4-d]pyrimidin-4 (3H)-one hydrochloride salt

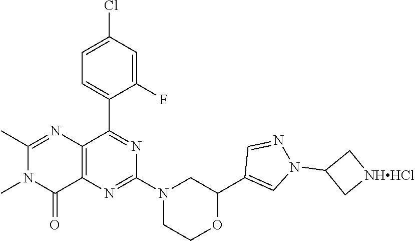

Procedure of step-2, example-3, was followed using tert-butyl 3-(4-(4-(4-(4-chloro-2-fluorophenyl)-6,7-dimethyl-8-oxo-7,8-dihydropyrimido[5,4-d]pyrimidin-2-yl)morpholin-2-yl)-1H-pyrazol-1-yl)azetidine-1-carboxylate as starting material to afford 6-(2-(1-(azetidin-3-yl)-1H-pyrazol-4-yl)morpholino)-8-(4-chloro-2-fluorophenyl)-2,3-dimethylpyrimido[5,4-d]pyrimidin-4 (3H)-one hydrochloride salt as yellow solid in 43.5% yield.

1H NMR (400 MHz, DMSO-d6) δ 9.09 (br s, 2H), 7.97 (s, 1H), 7.73 (s, 1H), 7.68-7.60 (m, 2H), 7.49-7.45 (m, 1H), 5.38-5.37 (m, 1H), 4.66-4.63 (m, 1H), 4.56-4.51 (m, 2H), 4.34 (br s, 4H), 4.03-4.01 (m, 1H), 3.50 (s, 3H), 3.21-3.11 (m, 2H), 2.44 (s, 3H).

LCMS Condition K: Rt=4.46 min, m/z 511.4 [M+H]⁺.

### Example 42

### 8-(4-chloro-2-fluorophenyl)-2,3-dimethyl-6-(2-(1-(1-methylazetidin-3-yl)-1H-pyrazol-4-yl)morpholino)pyrimido[5,4-d]pyrimidin-4 (3H)-one

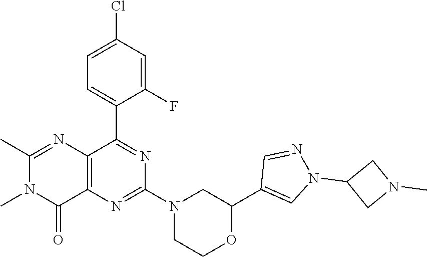

Procedure for synthesis of cpd-6a, was followed using example-41 and paraformaldehyde as starting materials to afford 8-(4-chloro-2-fluorophenyl)-2,3-dimethyl-6-(2-(1-(1-methylazetidin-3-yl)-1H-pyrazol-4-yl)morpholino)pyrimido[5,4-d]pyrimidin-4 (3H)-one after purification by reverse phase prep HPLC as yellow solid in 33.5% yield.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 65% A and 35% B in 3 min, then to 20% A and 80% B in 20 min., then to 5% A and 95% B in 21 min., held this composition up to 22 min. for column washing, then returned to initial composition in 23 min. and held till 25 min.

1H NMR (400 MHz, Methanol-d4) δ 7.84 (s, 1H), 7.69-7.59 (m, 2H), 7.39-7.30 (m, 2H), 5.03-4.87 (m, 2H), 4.75-4.66 (m, 1H), 4.59 (dd, J=2.7, 10.2 Hz, 1H), 4.09-4.01 (m, 1H), 3.90-3.83 (m, 2H), 3.86-3.70 (m, 1H), 3.63 (s, 3H), 3.64-3.55 (m, 2H), 3.31-3.14 (i, 2H), 2.52 (m, 3H), 2.47 (1, 3H).

LCMS Condition K: Rt=2.70 min m/z 525.4 [M+H]⁺.

### Synthesis of Example 43 to 58

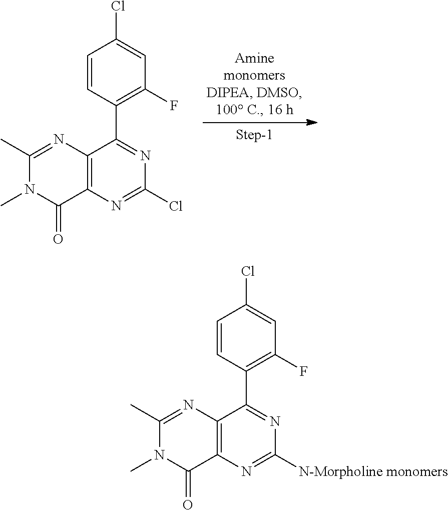

### Corresponding Morpholine Monomers

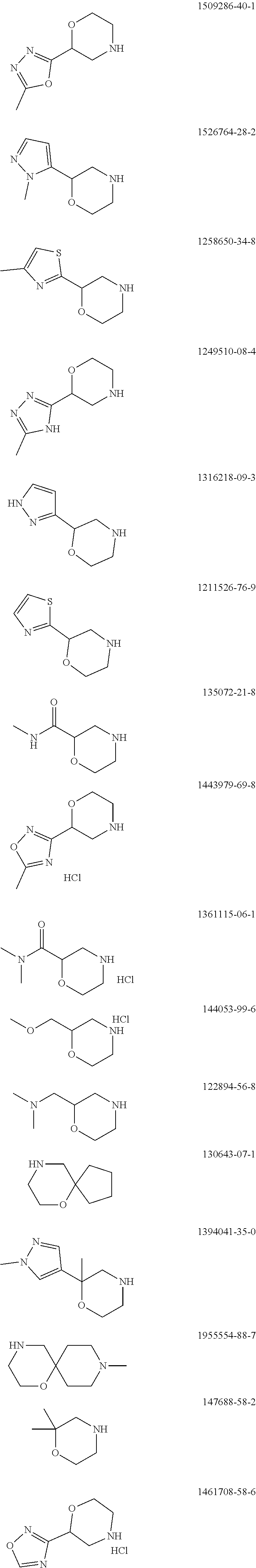

### General Procedure

To a stirred solution of 6-chloro-8-(4-chloro-2-fluorophenyl)-2,3-dimethylpyrimido[5,4-d]pyrimidin-4 (3H)-one (50 mg, 1 equiv) in DMSO (2 mL) was added DIPEA (3 equiv) followed by corresponding morpholine monomers (1.2 eq) at RT.

Resulting mixture was heated at 100° C. for 16 h. Reaction mixture was concentrated under reduced pressure. Crude mass was purified by reverse phase preparative HPLC to afford Example 43 to Example 58.

All the compounds (Ex. 43-58) were purified by below mentioned Prep-HPLC method Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 50% A and 50% B in 3 min, then to 20% A and 80% B in 20 min., then to 5% A and 95% B in 21 min., held this composition up to 22 min. for column washing, then returned to initial composition in 23 min. and held till 25 min.

| Ex. # | | Reagents/condi- |
|---|---|---|
| Structure | Name | tions |
| Ex. 43 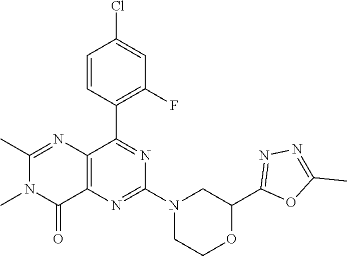 | 8-(4-chloro-2-fluorophenyl)- 2,3-dimethyl-6-(2-(5- methyl-1,3,4-oxadiazol-2- yl)morpholino)pyrimido[5,4- d]pyrimidin-4(3H)-one | Step-1 2-(5-methyl- 1,3,4-oxadiazol- 2-yl)morpholine |
| Ex. 44 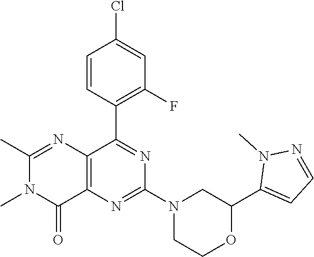 [SMILES: Cc1nc2c(-c3ccc(Cl)cc3F)nc(N3CCOC(c4ccnn4C)C3)nc2c(=O)n1C] | 8-(4-chloro-2-fluorophenyl)- 2,3-dimethyl-6-(2-(1- methyl-1H-pyrazol-5- yl)morpholino)pyrimido[5,4- d]pyrimidin-4(3H)-one | Step-1 2-(1-methyl-1H- pyrazol-5- yl)morpholine |
| Ex. 45 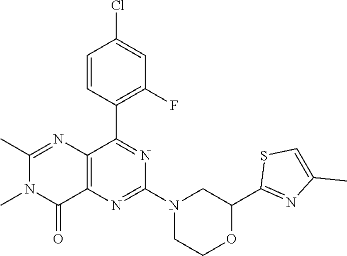 | 8-(4-chloro-2-fluorophenyl)- 2,3-dimethyl-6-(2-(4- methylthiazol-2- yl)morpholino)pyrimido[5,4- d]pyrimidin-4(3H)-one | Step-1 2-(4- methylthiazol-2- yl)morpholine |
| Ex. 46 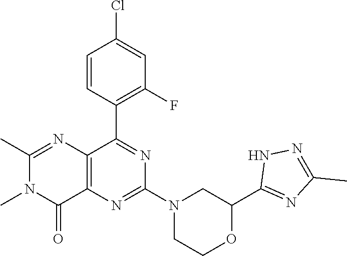 | 8-(4-chloro-2-fluorophenyl)- 2,3-dimethyl-6-(2-(3- methyl-1H-1,2,4-triazol-5- yl)morpholino)pyrimido[5,4- d]pyrimidin-4(3H)-one | Step-1 2-(5-methyl-4H- 1,2,4-triazol-3- yl)morpholine |
| Ex. 47 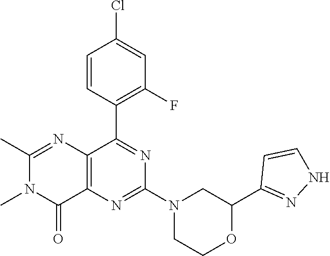 [SMILES: Cc1nc2c(-c3ccc(Cl)cc3F)nc(N3CCOC(c4cc[nH]n4)C3)nc2c(=O)n1C] | 6-(2-(1H-pyrazol-3- yl)morpholino)-8-(4-chloro- 2-fluorophenyl)-2,3- dimethylpyrimido[5,4- d]pyrimidin-4(3H)-one | Step-1 2-(1H-pyrazol-3- yl)morpholine |
| Ex. 48 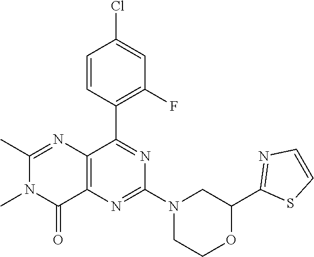 [SMILES: Cc1nc2c(-c3ccc(Cl)cc3F)nc(N3CCOC(c4nccs4)C3)nc2c(=O)n1C] | 8-(4-chloro-2-fluorophenyl)- 2,3-dimethyl-6-(2-(thiazol-2- yl)morpholino)pyrimido[5,4- d]pyrimidin-4(3H)-one | Step-1 2-(thiazol-2- yl)morpholine |
| Ex. 49 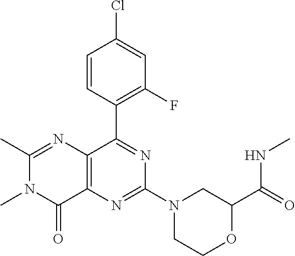 [SMILES: CNC(=O)C1CN(c2nc(-c3ccc(Cl)cc3F)c3nc(C)n(C)c(=O)c3n2)CCO1] | 4-(4-(4-chloro-2- fluorophenyl)-6,7-dimethyl- 8-oxo-7,8- dihydropyrimido[5,4- d]pyrimidin-2-yl)-N- methylmorpholine-2- carboxamide | Step-1 N- methylmorpholine- 2- carboxamide |
| Ex. 50 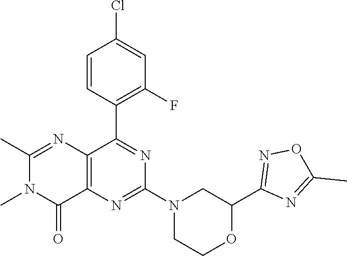 | 8-(4-chloro-2-fluorophenyl)- 2,3-dimethyl-6-(2-(5- methyl-1,2,4-oxadiazol-3- yl)morpholino)pyrimido[5,4- d]pyrimidin-4(3H)-one | Step-1 2-(5-methyl- 1,2,4-oxadiazol- 3-yl)morpholine hydrochloride |
| Ex. 51 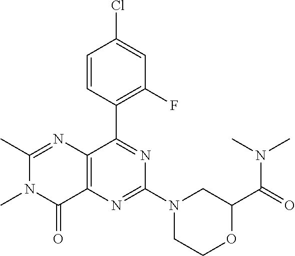 [SMILES: Cc1nc2c(-c3ccc(Cl)cc3F)nc(N3CCOC(C(=O)N(C)C)C3)nc2c(=O)n1C] | 4-(4-(4-chloro-2- fluorophenyl)-6,7-dimethyl- 8-oxo-7,8- dihydropyrimido[5,4- d]pyrimidin-2-yl)-N,N- dimethylmorpholine-2- carboxamide | Step-1 N,N- dimethylmorpholine- 2- carboxamide hydrochloride |
| Ex. 52 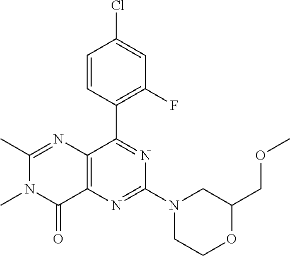 | 8-(4-chloro-2-fluorophenyl)- 6-(2- (methoxymethyl)morpholino)- 2,3-dimethylpyrimido[5,4- d]pyrimidin-4(3H)-one | Step-1 2- (methoxymethyl) morpholine hydrochloride |
| Ex. 53 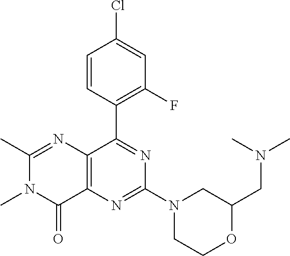 | 8-(4-chloro-2-fluorophenyl)- 6-(2- ((dimethylamino)methyl)mor- pholino)-2,3- dimethylpyrimido[5,4- d]pyrimidin-4(3H)-one | Step-1 N,N-dimethyl-1- (morpholin-2- yl)methanamine |
| Ex. 54 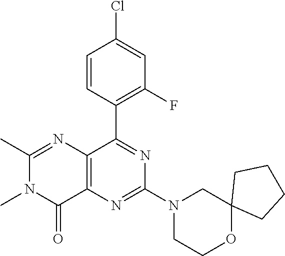 [SMILES: Cc1nc2c(-c3ccc(Cl)cc3F)nc(N3CCOC4(CCCC4)C3)nc2c(=O)n1C] | 8-(4-chloro-2-fluorophenyl)- 2,3-dimethyl-6-(6-oxa-9- azaspiro[4.5]decan-9- yl)pyrimido[5,4-d]pyrimidin- 4(3H)-one | Step-1 6-oxa-9- azaspiro[4.5]decane |
| Ex. 55 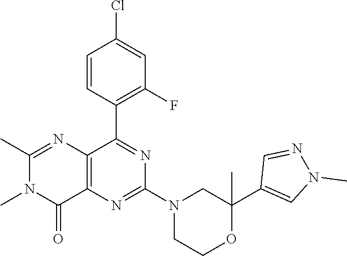 | 8-(4-chloro-2-fluorophenyl)- 2,3-dimethyl-6-(2-methyl-2- (1-methyl-1H-pyrazol-4- yl)morpholino)pyrimido[5,4- d]pyrimidin-4(3H)-one | Step-1 2-methyl-2-(1- methyl-1H- pyrazol-4- yl)morpholine |
| Ex. 56 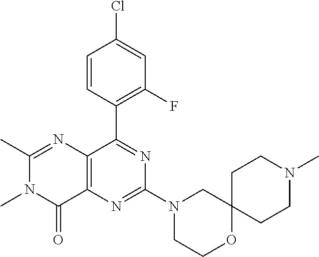 | 8-(4-chloro-2-fluorophenyl)- 2,3-dimethyl-6-(9-methyl-1- oxa-4,9- diazaspiro[5.5]undecan-4- yl)pyrimido[5,4-d]pyrimidin- 4(3H)-one | Step-1 9-methyl-1-oxa- 4,9- diazaspiro[5.5]un- decane dihydrochloride |
| Ex. 57 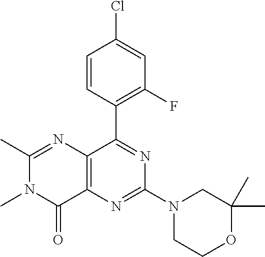 | 8-(4-chloro-2-fluorophenyl)- 6-(2,2-dimethylmorpholino)- 2,3-dimethylpyrimido[5,4- d]pyrimidin-4(3H)-one | Step-1 2,2- dimethylmorpholine |
| Ex. 58 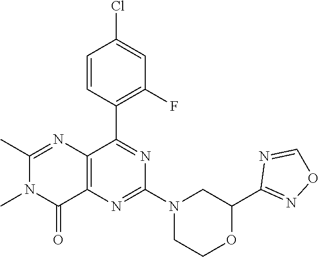 [SMILES: Cc1nc2c(-c3ccc(Cl)cc3F)nc(N3CCOC(c4ncon4)C3)nc2c(=O)n1C] | 6-(2-(1,2,4-oxadiazol-3- yl)morpholino)-8-(4-chloro- 2-fluorophenyl)-2,3- dimethylpyrimido[5,4- d]pyrimidin-4(3H)-one | Step-1 2-(1,2,4- oxadiazol-3- yl)morpholine hydrochloride |

### Analytical Data

| Ex. # | 1H-NMR | LCMS | Description | Yield (%) |
|---|---|---|---|---|
| Ex. 43 | ¹H-NMR (400 MHz, DMSO-d6): δ 7.67 (t, | Condition H: | Yellow | 35.8 |
| | J = 8.0 Hz, 1H), 7.62 (dd, J = 9.6 Hz, J = 1.6 | Rt = 3.13 min. | solid | |
| | Hz 1H), 7.47 (dd, J = 8.4 Hz, J = 1.6 Hz 1H), | m/z 472.3 | | |
| | 4.98 (dd, J = 9.6 Hz, J = 2.8 Hz, 1H), 4.74 | [M + H]⁺. | | |
| | (d, J = 12.8 Hz, 1H), 4.40 (d, J = 13.2 Hz, | | | |
| | 1H), 4.02 (d, J = 11.2 Hz, 1H), 3.79 (t, J = | | | |
| | 8.8 Hz, 1H), 3.64-3.58 (m, 1H), 3.51 (s, 3H), | | | |
| | 3.42-3.37 (m, 1H), 3.28(s, 3H), 2.44 (s, 3H). | | | |
| Ex. 44 | ¹H-NMR (400 MHz, CDCl₃): δ 7.61 (t, J = | Condition H: | Yellow | 50.5 |
| | 7.6 Hz, 1H), 7.44 (d, J = 1.6 Hz, 1H), 7.30- | Rt = 2.99 min. | solid | |
| | 7.29 (m, 1H), 7.27-7.25 (m, 1H), 6.32 (s, | m/z 470.3 | | |
| | 1H), 4.89 (d, J = 13.6 Hz, 1H), 4.71-4.65 | [M + H]⁺. | | |
| | (m, 2H), 4.05 (d, J = 11.6 Hz, 1H), 3.95 (s, | | | |
| | 3H), 3.85-3.78 (m, 1H), 3.63 (s, 3H), 3.60- | | | |
| | 3.54 (m, 1H), 3.50-3.44 (m, 1H), 2.53 (s, 3H). | | | |
| Ex. 45 | ¹H-NMR (400 MHz, DMSO-d6): δ 7.69-7.61 | Condition P: | Yellow | 37.6 |
| | (m, 2H), 7.44 (d, J = 8 Hz, 1H), 7.29 (s, 1H), | Rt = 2.91 min. | solid | |
| | 5.03 (d, J = 11.2 Hz, 1H), 4.87 (dd, J = 10.4 | m/z 487.1 | | |
| | Hz, J = 2.8 Hz, 1H), 4.57 (d, J = 12.8 Hz, | [M + H]⁺. | | |
| | 1H), 4.13 (d, J = 9.2 Hz, 1H), 3.84-3.78 (m, | | | |
| | 1H), 3.52 (s, 3H), 3.24-3.18 (m, 1H), 3.12- | | | |
| | 3.06 (m, 1H), 2.44 (s, 3H), 2.38 (s, 3H). | | | |
| Ex. 46 | ¹H-NMR (400 MHz, DMSO-d6): δ 13.6 (bs, | Condition H: | Yellow | 54.5 |
| | 1H), 7.66-7.57 (m, 2H), 7.42 (d, J = 8.8 Hz, | Rt = 2.95 min. | solid | |
| | 1H), 4.7 (bs, 1H), 4.51 (d, J = 10.8 Hz, 2H), | m/z 470.3 | | |
| | 4.01 (bs, 1H), 3.68 (bs, 1H), 3.48 (s, 3H), | [M + H]⁺. | | |
| | 3.28 (bs, 1H), 3.21-3.15 (m, 1H), 2.41 (s, | | | |
| | 3H), 2.30 (s, 3H). | | | |
| Ex. 47 | ¹H-NMR (400 MHz, DMSO-d6): δ 12.77 (s, | Condition p: | Yellow | 47.6 |
| | 1H), 7.70-7.59 (m, 3H), 7.44 (d, J = 8.4 Hz, | Rt = 2.56 min. | solid | |
| | 1H), 6.29 (s, 1H), 4.80-4.70 (m, 1H), 4.58- | m/z 456.1 | | |
| | 4.54 (m, 2H), 4.03 (d, J = 10.8 Hz, 1H), 3.70 | [M + H]⁺. | | |
| | (m, 1H), 3.51 (s, 3H), 3.25-3.19 (m, 2H), | | | |
| | 2.44 (s, 3H). | | | |
| Ex. 48 | ¹H-NMR (400 MHz, DMSO-d6): δ 7.83 (d, | Condition H: | Yellow | 40.8 |
| | J = 2.8 Hz, 1H), 7.76 (d, J = 3.2 Hz, 1H), | Rt = 3.20 min. | solid | |
| | 7.68 (t, J = 8.0 Hz, 1H), 7.63 (dd, J = 9.6 Hz, | m/z 473.3 | | |
| | J = 1.6 Hz, 1H), 7.48 (dd, J = 8.0 Hz, J = 1.6 | [M + H]⁺. | | |
| | Hz, 1H), 5.044 (d, J = 13.2 Hz, 1H), 4.95 | | | |
| | (dd, J = 10.4 Hz, J = 2.8 Hz, 1H), 4.57 (d, | | | |
| | J = 12.8 Hz, 1H), 4.14 (d, J = 11.6 Hz, 1H), | | | |
| | 3.83 (t, J = 11.6 Hz, 1H), 3.52 (s, 3H), 3.24- | | | |
| | 3.11 (m, 2H), 2.44 (s, 3H). | | | |
| Ex. 49 | ¹H-NMR (400 MHz, DMSO-d6): δ 7.87 (d, | Condition H: | Yellow | 51.6 |
| | J = 4.4 Hz, 1H), 7.67-7.60 (m, 2H), 7.46 (d, | Rt = 2.50 min. | solid | |
| | J = 8.4 Hz, 1H), 4.80 (d, J = 13.6 Hz, 1H), | m/z 447.1 | | |
| | 4.47 (d, J = 13.6 Hz, 1H), 4.07-3.98 (m, | [M + H]⁺. | | |
| | 2H), 3.64 (m, 1H), 3.51 (s, 3H), 3.12 (m, | | | |
| | 1H), 2.95 (t, J = 12.8 Hz, 1H), 2.62 (s, 3H), | | | |
| | 2.44 (s, 3H). | | | |
| Ex. 50 | ¹H-NMR (400 MHz, DMSO-d6): δ 7.68- | Condition H: | Yellow | 18.6 |
| | 7.59 (m, 2H), 7.47 (dd, J = 8.4 Hz, J = 2.0 | Rt = 3.23 min. | solid | |
| | Hz, 1H), 4.84 (dd, J = 9.6 Hz, J = 2.4 Hz, | m/z 472.3 | | |
| | 1H), 4.74 (d, J = 12.8 Hz, 1H), 4.46 (d, J = | [M + H]⁺. | | |
| | 13.2 Hz, 1H), 4.05 (d, J = 11.2 Hz, 1H), | | | |
| | 3.79-3.73 (m, 1H), 3.51 (s, 3H), 3.45-3.39 | | | |
| | (m, 1H), 3.35-3.34 (m, 1H), 2.61 (s, 3H), | | | |
| | 2.44 (s, 3H). | | | |
| Ex. 51 | ¹H-NMR (400 MHz, DMSO-d6): δ 7.67- | Condition H: | Yellow | 51.5 |
| | 7.60 (m, 2H), 7.45 (d, J = 8.4 Hz, 1H), 4.57 | Rt = 3.13 min. | solid | |
| | (d, J = 13.6 Hz, 1H), 4.48 (d, J = 13.6 Hz, | m/z 461.3 | | |
| | 1H), 4.31 (d, J = 8.0 Hz, 1H), 3.96 (d, J = | [M + H]⁺. | | |
| | 10.8 Hz, 1H), 3.66 (t, J = 9.6 Hz, 1H), 3.51 | | | |
| | (s, 3H), 3.27-3.14 (m, 1H), 3.12 (m, 1H), | | | |
| | 3.03 (s, 3H), 2.83 (s, 3H), 2.32 (s, 3H). | | | |
| Ex. 52 | ¹H-NMR (400 MHz, CDCl₃): δ 7.58 (t, J = | Condition P: | Yellow | 43.7 |
| | 8.0 Hz, 1H), 7.27-7.19 (m, 2H), 4.72 (t, J = | Rt = 2.64 min. | solid | |
| | 12.8 Hz, 2H), 4.06 (dd, J = 11.6 Hz, J = 3.2 | m/z 434.1 | | |
| | Hz, 1H), 3.70-3.64 (m, 2H), 3.60 (s, 3H), | [M + H]⁺. | | |
| | 3.54-3.51 (m, 2H), 3.40 (s, 3H), 3.20-3.13 | | | |
| | (m, 1H), 3.00-2.94 (m, 1H), 2.49 (s, 3H). | | | |
| Ex. 53 | ¹H-NMR (400 MHz, DMSO-d6): δ 7.66- | Condition H: | Yellow | 45.3 |
| | 7.59 (m, 2H), 7.45 (d, J = 8.4 Hz, 1H), 4.61 | Rt = 2.52 min. | solid | |
| | (d, J = 12.4 Hz, 1H), 4.47 (d, J = 13.6 Hz, | m/z 447.3 | | |
| | 1H), 3.93 (d, J = 9.6 Hz, 1H), 3.57-3.51 (m, | [M + H]⁺. | | |
| | 5H), 3.06 (t, J = 10.4 Hz, 1H), 2.77 (t, J = | | | |
| | 12.8 Hz, 1H), 2.43 (s, 3H), 2.40-2.33 (m, | | | |
| | 2H), 2.18 (s, 6H). | | | |
| Ex. 54 | ¹H-NMR (400 MHz, DMSO-d6): δ 7.66- | Condition H: | Yellow | 53.4 |
| | 7.58 (m, 2H), 7.46 (dd, J = 8.4 Hz, J = 2.0 | Rt = 3.45 min. | solid | |
| | Hz, 1H), 3.80-3.79 (m, 2H), 3.74 (s, 2H), | m/z 444.3 | | |
| | 3.70-3.67 (m, 2H), 3.50 (s, 3H), 2.42 (s, | [M + H]⁺. | | |
| | 3H), 1.72-1.67 (m, 4H), 1.65-1.57 (m, 4H). | | | |
| Ex. 55 | ¹H-NMR (400 MHz, DMSO-d6): δ 7.69- | Condition H: | Yellow | 46.8 |
| | 7.60 (m, 2H), 7.55 (s, 1H), 7.47 (d, J = 8.4 | Rt = 2.98 min. | solid | |
| | Hz, 1H), 7.32 (s, 1H), 4.59 (d, J = 13.2 Hz, | m/z 484.3 | | |
| | 1H), 4.16 (d, J = 13.6 Hz, 1H), 3.73 (s, 3H), | [M + H]⁺. | | |
| | 3.51 (s, 3H), 3.42-3.39 (m, 2H), 2.43 (s, | | | |
| | 3H), 2.07 (d, J = 5.2 Hz, 2H), 1.42 (s, 3H). | | | |
| Ex. 56 | ¹H-NMR (400 MHz, DMSO-d6): δ 7.67- | Condition H: | Yellow | 42.8 |
| | 7.59 (m, 2H), 7.47 (d, J = 6.4 Hz, 1H), 3.83 | Rt = 2.55 min. | solid | |
| | (s, 2H), 3.72 (t, J = 5.2 Hz, 4H), 3.50 (s, | m/z 473.3 | | |
| | 3H), 2.42 (s, 3H), 2.35-2.32 (m, 2H), 2.26- | [M + H]⁺. | | |
| | 2.21 (m, 2H), 2.14 (s, 3H), 1.73-1.70 (m, | | | |
| | 2H), 1.53 (t, J = 9.6 Hz, 2H). | | | |
| Ex. 57 | ¹H-NMR (400 MHz, DMSO-d6): δ 7.67- | Condition H: | Yellow | 48.5 |
| | 7.59 (m, 2H), 7.45 (d, J = 8.4 Hz, 1H), 3.79 | Rt = 3.23 min. | solid | |
| | (s, 2H), 3.72 (s, 2H), 3.66 (s, 2H), 3.50 (s, | m/z 418.3 | | |
| | 3H), 2.42 (s, 3H), 1.82 (s, 6H). | [M + H]⁺. | | |
| Ex. 58 | ¹H-NMR (400 MHz, DMSO-d6): δ 9.67 (s, | Condition H: | Yellow | 14.8 |
| | 1H), 7.66 (t, J = 8.0 Hz, 1H), 7.60 (d, J = 8.0 | Rt = 5.66 min. | solid | |
| | Hz, 1H), 7.45 (d, J = 8.0 Hz, 1H), 4.95 (dd, | m/z 415.3 | | |
| | J = 9.6 Hz, J = 2.4 Hz, 1H), 4.75 (d, J = | [M + H]⁺. | | |
| | 11.6 Hz, 1H), 4.46 (d, J = 13.6 Hz, 1H), | | | |
| | 4.06 (d, J = 11.6 Hz, 1H), 3.79 (t, J = 8.4 Hz, | | | |
| | 1H), 3.51 (s, 3H), 3.49-3.46 (m, 1H), 3.38- | | | |
| | 3.36 (m, 1H), 2.44 (s, 3H). | | | |

### Example 59: 8-(4-chloro-2-fluorophenyl)-3-(4-methoxybenzyl)-2-methyl-6-(2-(I-methyl-1H-pyrazol-4-yl)morpholino)pyrimido[5,4-d]pyrimidin-4 (3H)-one

Compound 59 was prepared following the procedure described in Example 1 steps 2-5, as follows

| | | | | | |
|---|---|---|---|---|---|
| | | | | Descrip- | Yield |
| Ex.# | Structure | Name | NMR/LCMS | tion | (%) |

| | | | | |
|---|---|---|---|---|
| Step-2 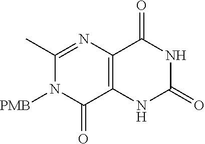 | 7-(4-methoxybenzyl)- 6-methyl-1,7- dihydropyrimido[5,4-d] pyrimidine-2,4,8(3H)- trione | ¹H-NMR (400 MHz, DMSO-d6): δ 11.18 (brs, 2H), 7.16 (d, J = 8.0 Hz, 2H), 5.2 (s, 2H) 3.65 (s, 3H) | light brown solid | 46.5 |
| Step-3 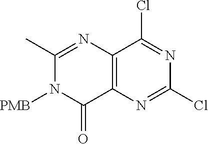 | 6,8-dichloro-3-(4- methoxybenzyl)-2- methylpyrimido[5,4-d] pyrimidin-4(3H)-one | LCMS Condition H: Rt = 3.39 min. m/z 351.0 [M + H]⁺. | Yellow solid | 18.0 |
| Step-4 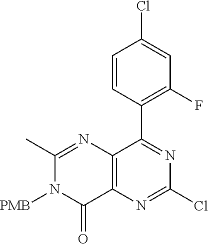 | 6-chloro-8-(4-chloro-2- fluorophenyl)-3-(4- methoxybenzyl)-2- methylpyrimido[5,4-d] pyrimidin-4(3H)-one | ¹H-NMR (400 MHz, CDCl3): δ 7.64 (t, J = 8.0 Hz, 1H), 7.30 (d, J = 12.0 Hz, 1H) 7.21 (d, J = 4.0 Hz, 1H), 7.20 (d, J = 8.0 Hz, 1H), 6.85 (d, J = 8.0 Hz, 1H) 5.33 (s, 2H), 3.77 (s, 3H), 2.55 (s, 3H), | Off white solid | 40.0 |
| Ex.59 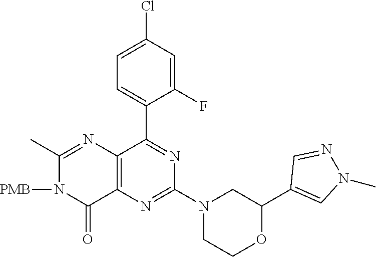 | 8-(4-chloro-2- fluorophenyl)-3-(4- methoxybenzyl)-2- methyl-6-(2-(1-methyl- 1H-pyrazol-4- yl)morpholino)pyrimido [5,4-d]pyrimidin-4(3H)- one | LCMS Condition R: Rt = 9.89 min. m/z 576.0 [M + H]⁺. | Yellow solid | 77.3 |
| PMB is paramethoxybenzyl | | | | |

Ex. 59 was obtained as a yellow solid in 77.3% yield.

¹H-NMR (400 MHz, DMSO-d6): δ 7.74 (s, 1H), 7.68 (t, J=8.0 Hz, 1H), 7.58 (d, J=7.2 Hz, 1H), 7.43 (d, J=7.2 Hz, 2H), 7.16 (d, J=8.4 Hz, 2H), 6.88 (d, J=8.4 Hz, 2H), 5.32 (s, 2H), 4.62 (d, J=12.4 Hz, 1H), 4.54-4.50 (m, 2H), 3.99 (d, J=11.2 Hz, 1H), 3.81 (s, 3H), 3.70 (s, 3H), 3.66-3.63 (m, 1H), 3.23-3.13 (m, 2H), 2.36 (s, 3H).

LCMS Condition R: Rt=9.89 min. m/z 576.0 [M+H]⁺.

### Example 60: 8-(4-chloro-2-fluorophenyl)-2-methyl-6-(2-(1-methyl-1H-pyrazol-4-yl)morpholino)pyrimido[5,4-d]pyrimidin-4 (3H)-one

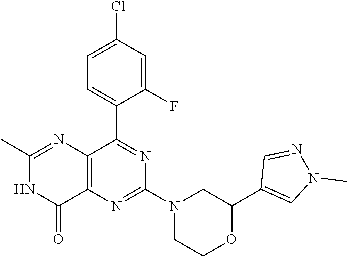

To a stirred solution of 8-(4-chloro-2-fluorophenyl)-3-(4-methoxybenzyl)-2-methyl-6-(2-(1-methyl-1H-pyrazol-4-yl)morpholino)pyrimido[5,4-d]pyrimidin-4 (3H)-one (Example 59, 80 mg, 0.14 mmol) in TFA (0.5 ml), was added Triflic acid (0.5 ml) at rt. Then the reaction mixture was stirred at rt for 16 h. Reaction mixture was neutralized with aq. NaHCO₃ solution and diluted with ethyl acetate, organic layer was separated, washed with brine solution and dried over anhydrous sodium sulfate. The filtrate was evaporated under reduced pressure to afford the crude which was purified through prep HPLC to afford 8-(4-chloro-2-fluorophenyl)-2-methyl-6-(2-(1-methyl-1H-pyrazol-4-yl)morpholino)pyrimido[5,4-d]pyrimidin-4 (3H)-one (44 mg, 69.5%) as yellowish solid. Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

¹H-NMR (400 MHz, DMSO-d6): δ 12.46 (s, 1H), 7.74 (s, 1H), 7.66-7.57 (m, 2H), 7.44-7.42 (m, 2H), 4.58 (d, J=12.8 Hz, 1H), 4.51-4.47 (m, 2H), 3.98 (d, J=11.2 Hz, 1H), 3.81 (s, 3H), 3.65 (t, J=11.2 Hz, 1H), 3.21-3.10 (m, 2H), 2.21 (s, 3H).

LCMS Condition P: Rt=2.40 min. m/z 456.0 [M+H]⁺.

### Example 61: 8-(4-chloro-2-fluorophenyl)-3-(2-methoxyethyl)-2-methyl-6-(2-(I-methyl-1H-pyrazol-4-yl)morpholino)pyrimido[5,4-d]pyrimidin-4 (3H)-one

Compounds 61 was prepared following the procedure described in Example 1B steps 2-5, as follows

| | | | | | Yield |
|---|---|---|---|---|---|
| Ex.# | Structure | Name | LCMS/NMR | Description | (%) |
| Step- 2 | 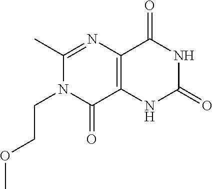 [SMILES: COCCn1c(C)nc2c(=O)[nH]c(=O)[nH]c2c1=O] | 7-(2-methoxyethyl)- 6-methyl-1,7- dihydropyrimido[5,4- d]pyrimidine- 2,4,8(3H)-trione | LCMS Condition A: Rt = 1.07 min. m/z 253.1 [M + H]⁺. | light brown solid | 50.3 |
| Step- 3 | 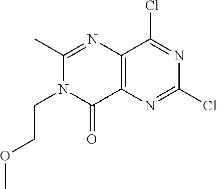 [SMILES: COCCn1c(C)nc2c(Cl)nc(Cl)nc2c1=O] | 6,8-dichloro-3-(2- methoxyethyl)-2- methylpyrimido[5,4- d]pyrimidin-4(3H)- one | LCMS Condition P: Rt = 1.58 min. m/z 289.1 [M+H]⁺ | Yellow solid | 17.5 |
| Step- 4 | 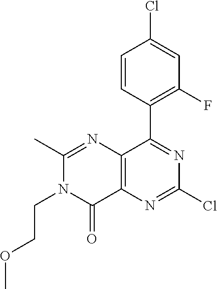 [SMILES: COCCn1c(C)nc2c(-c3ccc(Cl)cc3F)nc(Cl)nc2c1=O] | 6-chloro-8-(4- chloro-2- fluorophenyl)-3-(2- methoxyethyl)-2- methylpyrimido[5,4- d]pyrimidin-4(3H)- one | LCMS Condition H: Rt = 3.93 min. m/z 383.2 [M + H]⁺ | Off white solid | 40.0 |

| | | | | |
|---|---|---|---|---|
| Ex. 61 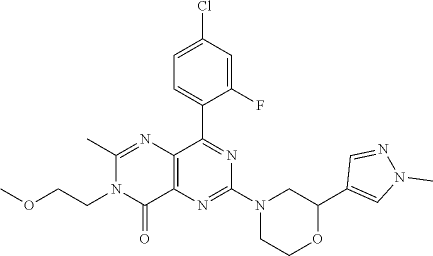 | 8-(4-chloro-2- fluorophenyl)-3-(2- methoxyethyl)-2- methyl-6-(2-(1- methyl-1H-pyrazol- 4- yl)morpholino)pyrimi- do[5,4-d]pyrimidin- 4(3H)-one | LCMS Condition P: Rt = 2.71 min. m/z 514.1 [M + H]⁺ | Yellow solid | 77.3 |

Ex. 61 was obtained as a yellow solid in 77.3% yield.

¹H-NMR (400 MHz, CDCl₃): δ 7.61 (t, J=8.0 Hz, 1H), 7.54 (s, 1H), 7.42 (s, 1H), 7.26-7.25 (m, 1H), 7.21-7.20 (m, 1H), 4.84 (d, J=12.4 Hz, 1H), 4.68 (d, J=13.2 Hz, 1H), 4.57 (dd, J=10.4 Hz, J=2.4 Hz, 1H),), 4.26 (t, J=5.2 Hz, 2H),), 4.05 (d, J=11.2 Hz, 1H), 3.89 (s, 3H), 3.79-3.70 (m, 3H), 3.33-3.30 (m, 1H), 3.27 (s, 3H), 3.22-3.19 (m, 1H), 2.57 (s, 3H).

LCMS Condition P: Rt=2.71 min m/z 514.1 [M+H]

### Preparation 1

### Preparation of 2-(1-methyl-1H-pyrazol-4-yl)morpholine (as HCl Salt)

### Step-1—Preparation of 2-chloro-1-(1-methyl-1H-pyrazol-4-yl)ethan-1-one

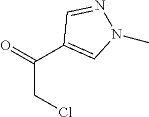

In an oven-dried two neck round-bottom flask 4-bromo-1-methyl-1H-pyrazole (10.0 g, 62.22 mmol) was dissolved in dry THF (100 mL) and cooled to −78° C. n-BuLi (40.0 mL, 93.2 mmol; 2.3 M in hexane) was slowly added to it under argon atmosphere and reaction was continued for at −78° C. for 1 h. A solution of 2-chloro-N-methoxy-N-methylacetamide (13.0 g, 93.2 mmol) in THF (40 mL) was then slowly added to it and reaction was prolonged for another one hour at 78° C. After completion, reaction mixture was quenched with saturated aqueous ammonium chloride solution and followed by extracted with ethyl acetate. Combined organic layer was dried over MgSO₄ and concentrated into vacuo. The crude was purified by column chromatography on silica gel (100-200 mesh size) using (20-50)% ethyl acetate in hexane as eluent to afford 2-chloro-1-(1-methyl-1H-pyrazol-4-yl)ethan-1-one (3.5 g, 35.5% yield) as off-white solid.

1H NMR (400 MHz, DMSO D6) δ 8.46 (s, 1H), 7.99 (s, 1H), 4.81 (s, 2H), 3.88 (s, 3H).

LCMS Condition A: Rt=1.48 min. m/z 159.02 [M+H]⁺.

### Step-2—Preparation of 2-(benzyl(2-hydroxyethyl)amino)-1-(1-methyl-1H-pyrazol-4-yl)ethan-1-one

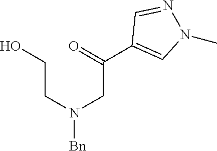 [SMILES: Cn1cc(C(=O)CN(CCO)Cc2ccccc2)cn1]

To a suspension of 2-chloro-1-(1-methyl-1H-pyrazol-4-yl)ethan-1-one (5.0 g, 31.6 mmol) in acetonitrile (50 mL) was added 2-(benzylamino)ethen-1-ol (5.75 g, 38.3 mmol) and potassium carbonate (8.8 g, 63.2) and heated the reaction mixture at 60° C. for 16 h. After completion excess solvent was removed under reduced pressure and the residue was with cold water and followed by extracted with ethyl acetate. Combined organic layer was dried over sodium sulfate and concentrated into vacuo to obtain the crude. The crude was purified by column chromatography on silica gel (100-200 mesh size) using (40-60)% ethyl acetate in hexane as eluent to afford 2-(benzyl(2-hydroxyethyl)amino)-1-(1-methyl-1H-pyrazol-4-yl)ethan-1-one (6.0 g, 69.5% yield) as yellow oil.

1H NMR (400 MHz, DMSO D6) δ 8.38 (s, 1H), 7.92 (s, 1H), 7.56-7.27 (m, 5H), 4.44 (t, J=5.04 Hz, 1H), 3.85 (s, 3H), 3.76 (s, 1H), 3.71 (s, 3H), 3.67 (s, 3H), 3.51-3.48 (m, 2H), 2.61 (t, J=6.32 Hz, 2H), LCMS Condition A: Rt=0.46 min. m/z 274.3 [M+H]⁺.

### Step-3—Preparation of 2-(benzyl(2-hydroxyethyl)amino)-1-(1-methyl-1H-pyrazol-4-yl)ethan-1-ol

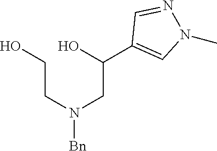 [SMILES: Cn1cc(C(O)CN(CCO)Cc2ccccc2)cn1]

To a stirred solution of 2-(benzyl(2-hydroxyethyl)amino)-1-(1-methyl-1H-pyrazol-4-yl)ethan-1-one (5.0 g, 18.32 mmol) was in dry Methanol and cooled the reaction mixture at 0° C. NaBH₄ (1.4 g, 3.67 mmol) was added to it portion-wise for 10 min and reaction was kept stirring for 15 min at 0° C. Reaction was then warm to room temperature for 2 h. The reaction mixture was quenched with cold water and followed by extracted with dichloromethane. Combined organic layer was dried over magnesium sulfate and concentrated into vacuo and purified by column chromatography on silica gel (100-200 mesh size) using (0-5)% methanol in dichloromethane as eluent to afford 2-(benzyl(2-hydroxyethyl)amino)-1-(1-methyl-1H-pyrazol-4-yl)ethan-1-ol (3.0 g, 59.5% yield) as yellow sticky oil.

1H NMR (400 MHz, DMSO D6) δ 7.47 (s, 1H), 7.35-7.29 (m, 5H), 7.26 (s, 1H). 4.76 (d, J=3.52 Hz, 1H), 4.62-4.58 (M, 1H), 4.38 (t, J=5.36 Hz, 1H), 3.75 (s, 3H), 3.69 (d, J=9.08 Hz, 2H), 3.48-3.4 (m, 2H), 2.63-2.54 (m, 3H).

LCMS Condition A: Rt=0.39 min. m/z 276.3 [M+H]⁺.

### Step-4—Preparation of 4-benzyl-2-(1-methyl-1H-pyrazol-4-yl)morpholine (as HCl Salt)

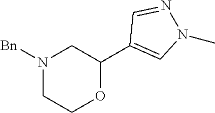 [SMILES: Cn1cc(C2CN(Cc3ccccc3)CCO2)cn1]

A mixture of afford 2-(benzyl(2-hydroxyethyl)amino)-1-(1-methyl-1H-pyrazol-4-yl)ethan-1-ol (2.0 g, 7.27 mmol) and aqueous HCl (6N, 20.0 mL) was heated to reflux for 2 h. The reaction mixture was evaporated under reduced pressure and the residue was washed with ether and dried under vacuum to afford 4-benzyl-2-(1-methyl-1H-pyrazol-4-yl)morpholine (1.7 g, 90.8% yield; HCl salt) as yellow sticky solid.

This material was forwarded for the next step without further purification.

1H NMR (400 MHz, DMSO D6) δ 12.05 (bs, 1H), 7.76 (s, 1H), 7.68-7.63 (m, 2H), 7.46-7.43 (m, 3H), 7.41 (s, 1H), 4.95 (d, J=10.0 Hz, 1H), 4.34 (s, 2H), 4.08-4.0 (m, 2H), 3.78 (s, 3H), 3.32 (d, J=11.8 Hz, 1H), 3.21 (d, J=12.12 Hz, 1H), 3.15-3.07 (m, 2H).

LCMS Condition A: Rt=0.40 min. m/z 258.3 [M+H]⁺.

### Step-5—Preparation of 2-(1-methyl-1H-pyrazol-4-yl)morpholine (as HCl Salt)

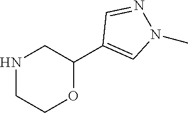

An ethanolic (50 mL) solution of 4-benzyl-2-(1-methyl-1H-pyrazol-4-yl)morpholine (2.0 g, 7.8 mmol) was taken in a par-autoclave vessel (100 mL) and purged it with argon. Pd(OH)₂ (0.5 g; 10% w/w) was added to it and reaction mixture was hydrogenated (30 psi) for 18 h at rt. The reaction mixture was filtered through a pad of celite and washed with ethanol. Evaporation of the solvent under reduced pressure afforded 2-(1-methyl-1H-pyrazol-4-yl)morpholine (1.2 g, 92.2% yield; HCl salt) as sticky solid.

1H NMR (400 MHz, DMSO D6) δ 9.82 (bs, 1H), 9.64 (bs, 1H), 7.77 (s, 1H), 7.45 (s, 1H), 4.75 (d, J=9.68 Hz, 1H), 3.98-3.86 (m, 2H), 3.78 (s, 3H), 3.31 (d, J=11.32 Hz, 1H), 3.31 (d, J=11.32 Hz, 1H), 3.18 3.31 (d, J=11.36 Hz, 1H), 3.15-2.85 (m, 2H).

LCMS Condition A: Rt=0.70 min. m/z 168.1 [M+H]⁺.

### Example 62: 8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one

Compounds 62 was prepared following the procedure described in Preparation 1 steps 2-5, and Step-6 was follows as described in Example 43-58.

| Ex.# | | | | |
|---|---|---|---|---|
| Structure | Name | LCMS/NMR | Description | Yield (%) |
| Step-2 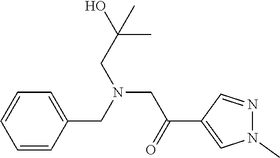 [SMILES: Cn1cc(C(=O)CN(Cc2ccccc2)CC(C)(C)O)cn1] | 2-(benzyl(2-hydroxy- 2- methylpropyl)amino)- 1-(1-methyl-1H- pyrazol-4-yl)ethan-1- one | LCMS Condition B: Rt = 1.402 min. m/z 302.1 [M + H]⁺. | light brown solid | 55.2 |
| Step-3 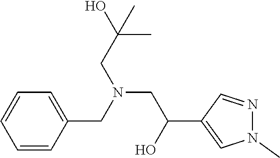 [SMILES: Cn1cc(C(O)CN(Cc2ccccc2)CC(C)(C)O)cn1] | 1-(benzyl(2-hydroxy- 2-(1-methyl-1H- pyrazol-4- yl)ethyl)amino)-2- methylpropan-2-ol | LCMS Condition B: Rt = 1.32 min. m/z 304.2 [M + H]⁺ | Yellow solid | 84.3 |
| Step-4 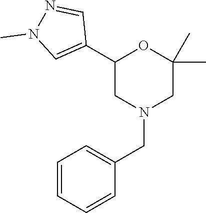 | 4-benzyl-2,2- dimethyl-6-(1-methyl- 1H-pyrazol-4- yl)morpholine (as HCl salt) | LCMS Condition B: Rt = 1.43 min. m/z 286.2 [M + H]⁺ | Off white solid | 79.6 |
| Step-5 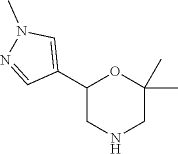 | 2,2-dimethyl-6-(1- methyl-1H-pyrazol-4- yl)morpholine (as HCl salt) | LCMS Condition A: Rt = 0.30 min. m/z 196.2 [M + H]⁺ | Yellow solid | 72.9 |
| Ex. 62 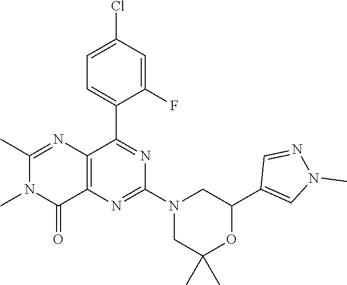 | 8-(4-chloro-2- fluorophenyl)-6-(2,2- dimethyl-6-(1-methyl- 1H-pyrazol-4- yl)morpholino)-2,3- dimethylpyrimido[5,4- d]pyrimidin-4(3H)- one | LCMS Condition H: Rt = 3.31 min. m/z 498.3 [M + H]⁺. | Yellow solid | 43.4 |

Ex. 62 was obtained a a yellow solid in 43.4% yield.

¹H-NMR (400 MHz, DMSO-d6): δ 7.69 (s, 1H), 7.65-7.56 (m, 2H), 7.44-7.40 (m, 2H), 4.74 (d, J=10.4 Hz, 1H), 4.69 (d, J=12.0 Hz, 1H), 4.49 (d, J=12.4 Hz, 1H), 3.78 (s, 3H), 3.48 (s, 3H), 2.98-2.88 (m, 2H), 2.40 (s, 3H), 1.21 (s, 6H).

LCMS Condition H: Rt=3.31 min. m/z 498.3 [M+H]⁺.

### Example 63: 8-(4-chloro-2-fluorophenyl)-2,3-dimethyl-6-[3-(1-methyl-1H-pyrazol-4-yl)pyrrolidin-1-yl]-3H,4H-pyrimido[5,4-d][1,3]diazin-4-one

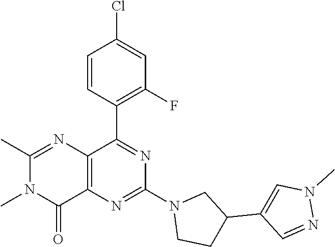

Compound 63 was prepared following the procedure described as described in Example 43-58.

Ex. 63 was obtained as a yellow solid in 80.2% yield.

1H NMR (400 MHz, DMSO D6) δ 7.66 (t, J=8.0 Hz, 1H), 7.54 (s, 1H), 7.48 (d, J=8.4 Hz, 1H), 7.42 (d, J=8.2 Hz, 1H), 7.34 (s, 1H), 4.05-4.04 (m, 1H), 3.81-3.79 (m, 1H), 3.78 (s, 3H), 3.66-3.63 (m, 1H), 3.47-3.43 (m, 2H), 2.44 (s, 3H), 2.34-2.32 (m, 1H), 2.04-2.02 (m, 1H).

LCMS Condition A: Rt=2.91 min. m/z 454.37 [M+H]⁺.

### Example 64: (R)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one

### Step-1—Preparation of tert-butyl (S)-3-(((4-(trifluoromethyl)phenyl)sulfonyl)oxy)pyrrolidine-1-carboxylate

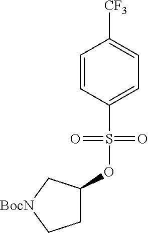 [SMILES: CC(C)(C)OC(=O)N1CC[C@H](OS(=O)(=O)c2ccc(C(F)(F)F)cc2)C1]

To a solution of tert-butyl (S)-3-hydroxypyrrolidine-1-carboxylate (500 mg, 2.67 mmol) and 4-trifluoromethylbenzenesulfonyl chloride (654 mg, 2.67 mmol) in DCM (5 ml), TEA (0.93 ml, 6.68 mmol) and DMAP (33 mg, 0.267 mmol) was added at 0° C. Then the reaction mixture was stirred at rt for 3 h. Then it was diluted with DCM and water.

Organic layer was separated, washed with brine solution, dried over anhydrous. sodium sulfate. It was evaporated under reduced pressure to afford the crude which was purified through column chromatography using 100-200 mesh silica gel to afford tert-butyl (S)-3-(((4-(trifluoromethyl)phenyl)sulfonyl)oxy)pyrrolidine-1-carboxylate (570 mg, 54.0%) as colorless solid.

¹H-NMR (400 MHz, DMSO-d6): δ 8.04 (d, J=8.0 Hz, 2H), 7.83 (d, J=8.0 Hz, 2H), 5.13 (s, 1H), 3.52-3.37 (m, 4H), 2.18-2.03 (m, 2H), 1.42 (s, 9H).

LCMS Condition H: Rt=3.64 min. m/z 396.0 [M+H]⁺.

### Step-2—Preparation of tert-butyl (R)-3-((1-methyl-1H-pyrazol-4-yl)oxy)pyrrolidine-1-carboxylate

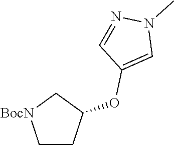 [SMILES: Cn1cc(O[C@@H]2CCN(C(=O)OC(C)(C)C)C2)cn1]

To a stirred solution of 1-methyl-1H-pyrazol-4-ol (156 mg, 1.59 mmol) in DMF (5 ml), was added cesium carbonate (705 mg, 2.16 mmol) and tert-butyl (S)-3-(((4-(trifluoromethyl)phenyl)sulfonyl)oxy)pyrrolidine-1-carboxylate (570 mg, 1.44 mmol) at RT. Then the reaction mixture was heated to 70° C. for 16 h. It was allowed to attend RT, diluted with ethyl acetate and ice-water. Organic layer was separated, washed with brine solution, dried over anhydrous sodium sulfate and filtered. The filtrate was evaporated under reduced pressure to afford the crude which was purified through column chromatography to afford tert-butyl (R)-3-((1-methyl-1H-pyrazol-4-yl)oxy)pyrrolidine-1-carboxylate (250 mg, 65.0%) as sticky gum.

¹H-NMR (400 MHz, DMSO-d6): δ 7.18 (s, 1H), 7.03 (s, 1H), 4.53 (bs, 1H), 3.81 (s, 3H), 3.62-3.47 (m, 4H), 2.16 (bs, 1H), 1.99 (bs, 1H), 1.45 (s, 9H).

LCMS Condition H: Rt=3.01 min. m/z 268.0 [M+H]⁺.

### Step-3—Preparation of (R)-1-methyl-4-(pyrrolidin-3-yloxy)-1H-pyrazole

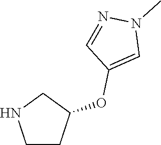

To a stirred solution of tert-butyl (R)-3-((1-methyl-1H-pyrazol-4-yl)oxy)pyrrolidine-1-carboxylate (250 mg, 0.935 mmol) in DCM (3 ml), was added 4M HCl in 1,4-dioxane (1.5 ml) at 0° C. The reaction mixture was stirred at RT for 3 h. It was dried under reduced pressure to afford (R)-1-methyl-4-(pyrrolidin-3-yloxy)-1H-pyrazole (130 mg, 68.0%) as sticky solid.

¹H-NMR (400 MHz, DMSO-d6): δ 7.52 (s, 1H), 7.22 (s, 1H), 4.72 (bs, 1H), 3.73 (s, 3H), 3.34-3.19 (m, 4H), 2.11-2.05 (m, 2H).

LCMS Condition H: Rt=0.62 min. m/z 168.0 [M+H]⁺.

### Step-4—Preparation of (R)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one

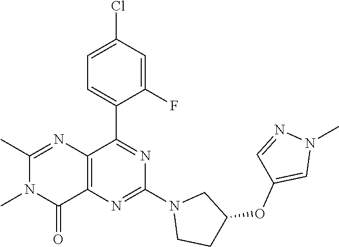

To a stirred solution of (R)-1-methyl-4-(pyrrolidin-3-yloxy)-1H-pyrazole (44 mg, 0.212 mmol) and 6-chloro-8-(4-chloro-2-fluorophenyl)-2,3-dimethylpyrimido[5,4-d]pyrimidin-4 (3H)-one (60 mg, 0.177 mmol) in DMSO was added DIPEA (0.12 ml, 0.708 mmol) at rt and allowed to heat at 70° C. for 16 h. LCMS showed the formation of desired product and purified through prep HPLC to afford (R)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one (61 mg, 73.4%) as yellowish solid.

Preparative HPLC was done on Waters auto purification instrument. Column name: YMC-Actus 018 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 mdi.

¹H-NMR (400 MHz, DMSO-d6): δ7.66-7.58 (m, 2H), 7.51 (s, 1H), 7.44 (d, J=8.0 Hz, 1H), 7.20 (s, 1H), 4.79 (s, 1H), 3.79-3.73 (m, 6H), 3.63-3.57 (n, 1H), 3.50 (c, 3H), 2.42 (p, 3H), 2.22 (bs, 2H).

LCMS Condition P: Rt=2.65 min m/z 470.1 [M+H]⁺.

### Example 65 to Example 68

Example 65 to Example 68 were synthesized following similar protocol as used for synthesis of example 14:

Details of Example 65 to example 68 are captured in following table:

| | | | | |
|---|---|---|---|---|
| | | | Intermediate | |
| Compound | | | compounds/SMs | Chiral separation |
| No. | Structure | Name | used | specification |

| | | | |
|---|---|---|---|
| Ex. 65 (Peak 1) 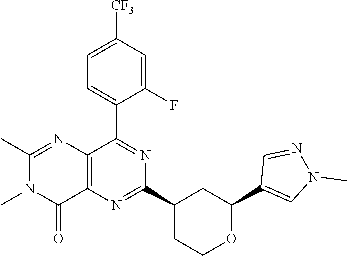 | 8-(2-fluoro-4- (trifluoromethyl)phe- nyl)-2,3-dimethyl-6- ((2S,4R)-2-(1- methyl-1H-pyrazol- 4-yl)tetrahydro-2H- pyran-4- yl)pyrimido[5,4- d]pyrimidin-4(3H)- one | 6-chloro-8-(2- fluoro-4- (trifluoromethyl) phenyl)-2,3- dimethylpyrimi- do[5,4- d]pyrimidin- 4(3H)-one (Ex 10, 3g) | Similar specifications as mentioned in step-6 of example-1. Absolute stereochemistry unknown |
| Ex. 66 (Peak 2) 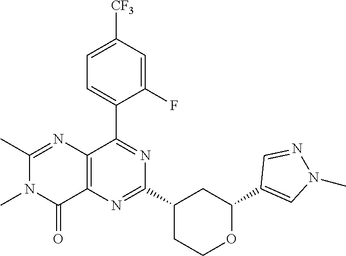 | 8-(2-fluoro-4- (trifluoromethyl)phe- nyl)-2,3-dimethyl-6- ((2R,4S)-2-(1- methyl-1H-pyrazol- 4-yl)tetrahydro-2H- pyran-4- yl)pyrimido[5,4- d]pyrimidin-4(3H)- one | 8-(2-fluoro-4- (trifluoromethyl) phenyl)-2,3- dimethyl-6-(2- (1-methyl-1H- pyrazol-4- yl)tetrahydro- 2H-pyran-4- yl)pyrimido[5,4- d]pyrimidin- 4(3H)-one[cis- racemate] | Absolute stereochemistry unknown |
| Ex. 67 (Peak 1) 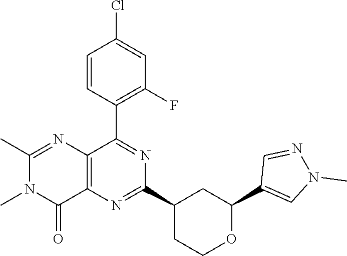 | 8-(4-chloro-2- fluorophenyl)-2,3- dimethyl-6- ((2S,4R)-2-(1- methyl-1H-pyrazol- 4-yl)tetrahydro-2H- pyran-4- yl)pyrimido[5,4- d]pyrimidin-4(3H)- one | Example 17 | Similar specifications as mentioned in step-6 of example-1. Absolute stereochemistry unknown |
| Ex. 68 (Peak 2) 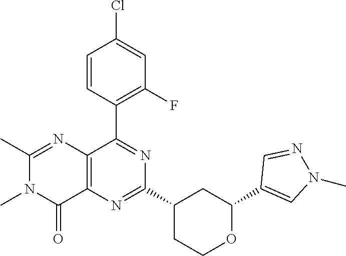 | 8-(4-chloro-2- fluorophenyl)-2,3- dimethyl-6- ((2R,4S)-2-(1- methyl-1H-pyrazol- 4-yl)tetrahydro-2H- pyran-4- yl)pyrimido[5,4- d]pyrimidin-4(3H)- one | | Absolute stereochemistry unknown |

Following table describes analytical data analysis and yield information of examples 65 to 68:

| Compound | | | Analytical chiral | | Yield |
|---|---|---|---|---|---|
| No. | HNMR | LCMS | HPLC | Description | (%) |
| Ex. 65 | 1H NMR (400 MHz, DMSO-d6) δ | Condition | Chiralpak IC (4.6 × | Off- | 45.4 |
| (Peak1) | 7.94-7.85 (m, 2H), 7.79 (d, J = | K: Rt = | 250 mm), 5 μ | white | |
| | 8.0 Hz, 1H), 7.63 (s, 1H), 7.36 (s, | 3.09 | Mobile Phase: | solid | |
| | 1H), 4.51 (dd, J = 2.1, 11.4 Hz, | min. m/z | Hexane/DCM/ | | |
| | 1H), 4.13-4.04 (m, 1H), 3.78 (s, | 503.4 | EtOH/IPAmine: | | |
| | 3H), 3.75-3.64 (m, 1H), 3.56 (s, | [M + H]⁺. | 50/25/25/0.1 | | |
| | 3H), 3.51-3.38 (m, 1H), 2.52 (s, | | Flow Rate: 1.0 | | |
| | 3H), 2.27-2.19 (m, 1H), 2.04- | | ml/min | | |
| | 1.80 (m, 3H). | | Solubility: MeOH | | |
| Ex. 66 | 1H NMR (400 MHz, DMSO-d6) δ | Condition | Rt = 6.96 min for | White | 41.1 |
| (Peak2) | 7.94-7.86 (m, 2H), 7.79 (d, J = | K: Rt = | Peak 1 and Rt = | solid | |
| | 8.2 Hz, 1H), 7.63 (s, 1H), 7.36 (s, | 3.09 | 7.64 min for | | |
| | 1H), 4.51 (d, J = 10.7 Hz, 1H), | min. m/z | Peak 2. | | |
| | 4.09 (dd, J = 3.1, 10.6 Hz, 1H), | 503.4 | | | |
| | 3.78 (s, 3H), 3.76-3.65 (m, 1H), | [M + H]⁺. | | | |
| | 3.56 (s, 3H), 3.50-3.39 (m, 1H), | | | | |
| | 2.52 (s, 3H), 2.25-2.21 (m, 1H), | | | | |
| | 2.05-1.75 (m, 3H). | | | | |
| Ex. 67 | 1H-NMR (400 MHz, DMSO-d6): δ | Condition | Chiralpak IC (4.6 × | White | 44.7 |
| (Peak1) | 7.70 (t, J = 8.0 Hz, 1H), 7.65-7.63 | K: Rt = | 250 mm), 5 μ | solid | |
| | (m, 2H), 7.50-7.48 (m, 1H), 7.36 | 3.10 | Mobile Phase: | | |
| | (s, 1H), 4.49 (d, J = 11.2 Hz, 1H), | min. m/z | Hexane/DCM/ | | |
| | 4.10-4.06 (m, 1H), 3.77 (s, 3H), | 469.3 | EtOH/IPAmine: | | |
| | 3.69 (t, J = 11.6 Hz, 1H), 3.55 (s, | [M + H]⁺. | 50/25/25/0.1 | | |
| | 3H), 3.45-3.39 (m, 1H), 2.52 (s, | | Flow Rate: 1.0 | | |
| | 3H), 2.20 (d, J = 13.2 Hz, 1H), | | Solubility: MeOH | | |
| | 1.99-1.87 (m, 3H). | | Rt = 8.02 min for | | |
| Ex. 68 | 1H-NMR (400 MHz, DMSO-d6): δ | Condition | Peak 1 and Rt = | | |
| (Peak2) | 7.70 (t, J = 8.0 Hz, 1H), 7.65-7.63 | K: Rt = | 8.89 min for | | |
| | (m, 2H), 7.50-7.48 (m, 1H),, 7.35 | 3.10 | Peak 2. | | |
| | (s, 1H), 4.48 (d, J = 11.2 Hz, 1H), | min. m/z | | | |
| | 4.10-4.07 (m, 1H), 3.77 (s, 3H), | 469.3 | | | |
| | 3.69 (t, J = 11.6 Hz, 1H), 3.55 (s, | [M + H]⁺. | | | |
| | 3H), 3.44-3.38 (m, 1H), 2.51 (s, | | | | |
| | 3H), 2.20 (d, J = 12.0 Hz, 1H), | | | | |
| | 1.99-1.84 (m, 3H). | | | | |

### Example 69

### 8-(4-chloro-2-fluorophenyl)-6-(2-(1-(2-methoxyethyl)-1H-pyrazol-4-yl)morpholino)-2,3-dimethylpyrimido[5,4-d]pyrimidin-4 (3H)-one

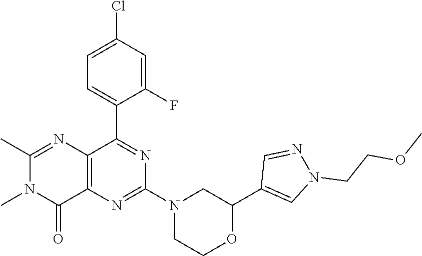

To a stirred solution of 6-(2-(1H-pyrazol-4-yl)morpholino)-8-(4-chloro-2-fluorophenyl)-2,3-dimethylpyrimido[5,4-d]pyrimidin-4 (3H)-one [Example-39](100.0 mg, 0.219 mmol) in DMF (2 mL) was added cesium carbonate (107.028 mg, 0.329 mmol) and 1-bromo-2-methoxyethane (0.031 mL, 0.329 mmol). Resulting mixture was heated at 110° C. for 16 h. LCMS showed the formation of desired product and purified through prep HPLC to afford 8-(4-chloro-2-fluorophenyl)-6-(2-(1-(2-methoxyethyl)-1H-pyrazol-4-yl)morpholino)-2,3-dimethylpyrimido[5,4-d]pyrimidin-4 (3H)-one (40 mg, 35.7% yield)

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 75% A and 35% B in 3 min, then to 20% A and 80% B in 20 min., then to 5% A and 95% B in 21 min., held this composition up to 22 min. for column washing, then returned to initial composition in 23 min. and held till 25 min.

1H NMR (400 MHz, DMSO) δ 7.77 (s, 1H), 7.71-7.57 (m, 2H), 7.50-7.42 (m, 2H), 4.62 (d, J=13.1 Hz, 1H), 4.51 (dd, J=2.4, 10.6 Hz, 2H), 4.23 (t, J=5.3 Hz, 2H), 4.00 (d, J=11.1 Hz, 1H), 3.71-3.60 (m, 3H), 3.51 (s, 3H), 3.24-3.11 (m, 5H), 2.44 (s, 3H).

LCMS Condition K: Rt=3.18 min. m/z 514.3 [M+H]+.

### Example 70

### 8-(2-chloro-4-fluorophenyl)-2,3-dimethyl-6-(2-(1-methyl-1H-pyrazol-4-yl)morpholino)pyrimido[5,4-d]pyrimidin-4 (3H)-one

### Step-1—Preparation of 6-chloro-8-(2-chloro-4-fluorophenyl)-2,3-dimethylpyrimido[5,4-d]pyrimidin-4 (3H)-one

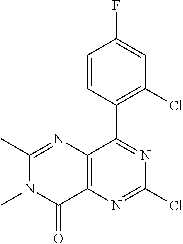 [SMILES: Cc1nc2c(-c3ccc(F)cc3Cl)nc(Cl)nc2c(=O)n1C]

To a stirred solution of 6,8-dichloro-2,3-dimethylpyrimido[5,4-d]pyrimidin-4 (3H)-one (100 mg, 0.41 mmol) and (2-chloro-4-fluorophenyl)boronic acid (71.311 mg, 0.41 mmol) in DME (4 mL) and water (1 mL) was added sodium carbonate (174 mg, 1.23 mmol) and degassed with argon. PdCl2(dpp)·DCM (6.694 mg, 0.0080 mmol) was added under inert atmosphere. The resulting mixture was heated at 90° C. for 4 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (35% ethyl acetate-hexane) to afford 6-chloro-8-(2-chloro-4-fluorophenyl)-2,3-dimethylpyrimido[5,4-d]pyrimidin-4 (3H)-one (55 mg, 39.5% yield) as light brown solid.

1H NMR (400 MHz, DMSO D6) δ 7.69-7.54 (2H, m), 7.45-7.41 (1H, m), 3.58 (3H, s), 2.54 (3H, s)

LCMS Condition A: Rt=2.14 min. m/z 339.2 [M+H]⁺.

### Step-2—Preparation of 8-(2-chloro-4-fluorophenyl)-2,3-dimethyl-6-(2-(1-methyl-1H-pyrazol-4-yl)morpholino)pyrimido[5,4-d]pyrimidin-4 (3H)-one

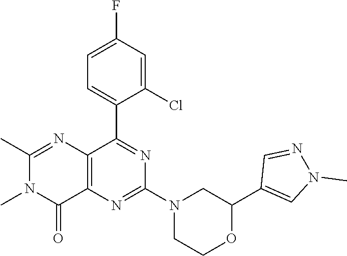

Procedure of Step-5, example-1 was followed using afford 6-chloro-8-(2-chloro-4-fluorophenyl)-2,3-dimethylpyrimido[5,4-d]pyrimidin-4 (3H)-one and 2-(1-methyl-1H-pyrazol-4-yl)morpholine hydrochloride salt as starting materials followed by reverse phase prep-HPLC purification to afford 8-(2-chloro-4-fluorophenyl)-2,3-dimethyl-6-(2-(1-methyl-1H-pyrazol-4-yl)morpholino)pyrimido[5,4-d]pyrimidin-4 (3H)-one as yellow solid in 42.5% yield.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 60% A and 40% B in 3 min, then to 20% A and 80% B in 20 min., then to 5% A and 95% B in 21 min., held this composition up to 22 min. for column washing, then returned to initial composition in 23 min. and held till 25 min.

1H NMR (400 MHz, DMSO-d6) δ 7.74 (s, 1H), 7.63-7.55 (m, 2H), 7.45 (s, 1H), 7.41-7.31 (m, 1H), 4.60 (d, J=12.6 Hz, 1H), 4.49 (d, J=7.8 Hz, 2H), 3.99 (d, J=10.6 Hz, 1H), 3.81 (s, 3H), 3.71-3.60 (m, 1H), 3.51 (s, 3H), 3.23-3.10 (m, 2H), 2.41 (s, 3H).

LCMS Condition K: Rt=2.81 min. m/z 470.3 [M+H]⁺.

### Example 71A—Enantiomer 1

### 2,3-dimethyl-6-((R)-2-(1-methyl-1H-pyrazol-4-yl)morpholino)-8-((1r,4R)-4-methylcyclohexyl)pyrimido[5,4-d]pyrimidin-4 (3H)-one

### Example 71B—Enantiomer 2

### 2,3-dimethyl-6-((S)-2-(1-methyl-1H-pyrazol-4-yl)morpholino)-8-((1r,4S)-4-methylcyclohexyl)pyrimido[5,4-d]pyrimidin-4 (3H)-one

Example 71A (Peak1) and 71B (Peak2) were obtained by chiral separation of Example-12B using similar specifications as mentioned in step-6 of example-1.

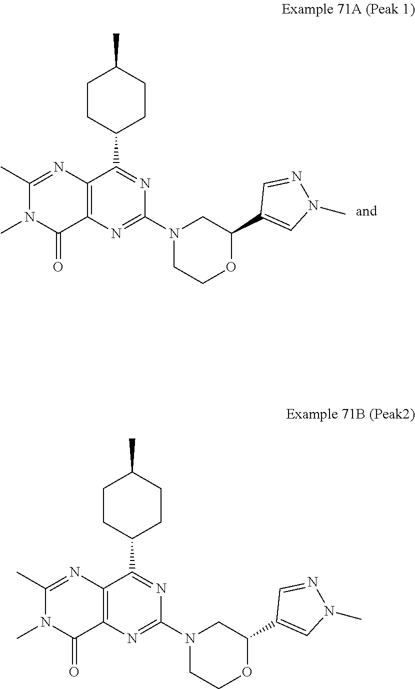

Example 71A (Peak1, Absolute stereochemistry unknown) 1H NMR (400 MHz, DMSO) δ 7.75 (s, 1H), 7.45 (s, 1H), 4.61 (d, J=13.1 Hz, 1H), 4.55-4.44 (m, 2H), 4.03-3.95 (m, 1H), 3.82 (s, 3H), 3.68-3.52 (m, 2H), 3.49 (s, 3H), 3.19-3.05 (m, 2H), 2.53 (s, 3H), 1.79 (t, J=11.3 Hz, 4H), 1.64-1.50 (m, 2H), 1.48-1.35 (m, 1H), 1.17-1.02 (m, 2H), 0.92 (d, J=6.5 Hz, 3H).

LCMS Condition K: Rt=3.41 min. m/z 438.4 [M+H]⁺.

Example 71B (Peak2, Absolute stereochemistry unknown) 1H NMR (400 MHz, DMSO) δ 7.75 (s, 1H), 7.46 (s, 1H), 4.65-4.57 (m, 1H), 4.55-4.44 (m, 2H), 4.03-3.95 (m, 1H), 3.82 (s, 3H), 3.68-3.52 (m, 2H), 3.50 (s, 3H), 3.19-3.05 (m, 2H), 2.53 (s, 3H), 1.79 (t, J=11.3 Hz, 4H), 1.64-1.51 (m, 2H), 1.48-1.34 (m, 1H), 1.17-1.03 (m, 2H), 0.92 (d, J=6.5 Hz, 3H).

LCMS Condition K: Rt=3.41 min. m/z 438.4 [M+H]⁺.

Analytical chiral HPLC: Chiralpak IC (4.6×250 mm), 5μ Mobile Phase: Hexane/DCM/EtOH/IPAmine—60/20/20/0.1 Flow Rate: 1.0 ml/min. Solubility: MeOH, Rt=10.91 min for Peak 1 and Rt=12.95 min for Peak 2

### Example 72

### 8-(6,6-difluorospiro[3.3]heptan-2-yl)-2,3-dimethyl-6-(2-(1-methyl-1H-pyrazol-4-yl)morpholino)pyrimido[5,4-d]pyrimidin-4 (3H)-one

### Step-1—Preparation of methyl 6,6-difluorospiro[3.3]heptane-2-carboxylate

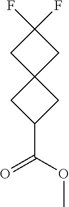

To a stirred solution of methyl 6-oxospiro[3.3]heptane-2-carboxylate (300 mg, 1.786 mmol) in DCM (4 mL) at 0° C. was added diethylaminosulfur trifluoride (0.708 ml, 5.357 mmol) dropwise. The resulting mixture was stirred at RT for 16 h. The reaction mixture was quenched with saturated aqueous solution of NaHCO₃ and extracted with DCM. Combined organic part was washed with water, brine, dried over anhydrous sodium sulphate, filtered and concentrated under reduced pressure. Crude mass was purified by column chromatography (20% ethyl acetate-hexane) to afford methyl 6,6-difluorospiro[3.3]heptane-2-carboxylate (300 mg. 88.3% yield) as colourless gum.

1H NMR (400 MHz, DMSO-d6) b 3.68 (s, 3H), 3.17-3.07 (m, 4H), 2.58-2.41 (m, 4H)

### Step-2—Preparation of 6,6-difluorospiro[3.3]heptane-2-carboxylic acid

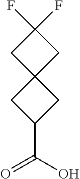 [SMILES: O=C(O)C1CC2(C1)CC(F)(F)C2]

To a stirred solution of methyl 6,6-difluorospiro[3.3]heptane-2-carboxylate (300 mg, 1.579 mmol) in THF (1.5 ml) and H2O (0.5 ml) was added lithium hydroxide ((211.768 mg, 8.842 mmol) portion wise at 5° C. The resulting mixture was stirred at RT for 16 h. Reaction mixture was concentrated under reduced pressure, diluted with water and washed with diethyl ether. Aqueous portion was cooled to 10° C., acidified with saturated aqueous NaHSO4 solution and extracted with dichloromethane. Organic part was dried over anhydrous sodium sulphate, filtered and concentrated under reduced pressure to afford 6,6-difluorospiro[3.3]heptane-2-carboxylic acid (270 mg. 97.7% yield) as colourless gum.

1H NMR (400 MHz, CDCl3) b 3.11-3.05 (m, 1H), 2.72-2.52 (m, 4H), 2.47-2.34 (m, 4H) [Carboxylic acid proton did not show up.]

GCMS Rt=8.64 min. ms (−) 175.1

### Step-3—Preparation of 6-chloro-8-(6,6-difluorospiro[3.3]heptan-2-yl)-2,3-dimethylpyrimido[5,4-d]pyrimidin-4 (3H)-one

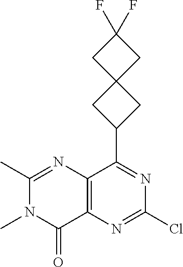 [SMILES: Cc1nc2c(C3CC4(C3)CC(F)(F)C4)nc(Cl)nc2c(=O)n1C]

To a stirred solution of 6,6-difluorospiro[3.3]heptane-2-carboxylic acid (600 mg, 2.459 mmol) in DCM (6 ml) and H2O (6 ml) were added AgNO3 ((62.657 mg, 0.369 mmol) and K2S2O8 (531.797 mg, 1.967 mmol) at 0° C. The resulting mixture was stirred at RT for 16 h. Reaction mixture was diluted with water and extracted with dichloromethane. Organic part was dried over anhydrous sodium sulphate, filtered and concentrated under reduced pressure. Crude mass was purified by reverse phase prep HPLC to afford 6-chloro-8-(6,6-difluorospiro[3.3]heptan-2-yl)-2,3-dimethylpyrimido[5,4-d]pyrimidin-4 (3H)-one (45 mg. 5.4% yield) as off-white solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 50% A and 50% B in 3 min, then to 10% A and 90% B in 20 min., then to 5% A and 95% B in 21 min., held this composition up to 22 min. for column washing, then returned to initial composition in 23 min. and held till 25 min.

1H NMR (400 MHz, DMSO-d6) δ 4.43-4.34 (m, 1H), 3.53 (s, 3H), 2.83-2.67 (m, 2H), 2.61-2.56 (m, 6H), 2.52-2.51 (m, 2H)

LCMS Condition K: Rt=2.11 min. m/z 341.3 [M+H]⁺.

### Step-3—Preparation of 8-(6,6-difluorospiro[3.3]heptan-2-yl)-2,3-dimethyl-6-(2-(1-methyl-1H-pyrazol-4-yl)morpholino)pyrimido[5,4-d]pyrimidin-4 (3H)-one

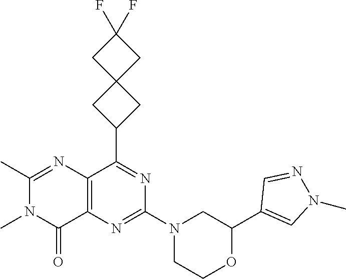

Procedure of Step-5, example-1 was followed using 6-chloro-8-(6,6-difluorospiro[3.3]heptan-2-yl)-2,3-dimethylpyrimido[5,4-d]pyrimidin-4 (3H)-one and 2-(1-methyl-1H-pyrazol-4-yl)morpholine hydrochloride salt as starting materials followed by reverse phase prep-HPLC purification to afford 8-(6,6-difluorospiro[3.3]heptan-2-yl)-2,3-dimethyl-6-(2-(1-methyl-1H-pyrazol-4-yl)morpholino)pyrimido[5,4-d]pyrimidin-4 (3H)-one as yellow solid in 51.5% yield.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 45% A and 55% B in 3 min, then to 30% A and 70% B in 20 min., then to 5% A and 95% B in 21 min., held this composition up to 22 min. for column washing, then returned to initial composition in 23 min. and held till 25 min.

1H NMR (400 MHz, CDCl3) δ 7.55 (s, 1H), 7.44 (s, 1H), 4.88 (d, J=13.1 Hz, 1H), 4.72 (d, J=13.3 Hz, 1H), 4.56 (dd, J=2.8, 10.2 Hz, 1H), 4.40-4.27 (m, 1H), 4.08 (d, J=11.6 Hz, 1H), 3.91 (s, 3H), 3.82-3.71 (m, 1H), 3.59 (s, 3H), 3.35-3.15 (m, 2H), 2.74 (t, J=12.3 Hz, 2H), 2.63-2.56 (m, 4H), 2.56-2.44 (m, 5H).

LCMS Condition K: Rt=2.94 min. m/z 472.4 [M+H]+.

### Example 73

### 8-(4-chloro-2-fluorophenyl)-2,3-dimethyl-6-(2-(1-methyl-1H-1,2,3-triazol-4-yl)morpholino)pyrimido[5,4-d]pyrimidin-4 (3H)-one

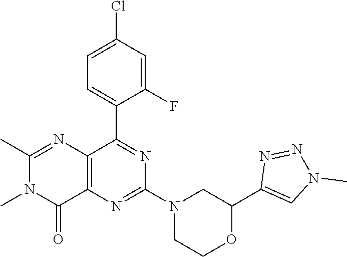

Example 73 was prepared using similar procedures described in Example 2 and Example 35. Example 73 was obtained as yellow solid in 32.8% yield.

1H NMR (400 MHz, DMSO-d6) δ 8.10 (s, 1H), 7.72-7.57 (m, 2H), 7.46 (d, J=8.3 Hz, 1H), 4.86-4.77 (m, 1H), 4.76-4.68 (m, 1H), 4.56 (d, J=13.8 Hz, 1H), 4.05 (s, 4H), 3.79-3.68 (m, 1H), 3.52 (s, 3H), 3.29-3.15 (m, 2H), 2.44 (s, 3H).

LCMS Condition K: Rt=3.05 min. m/z 471.3 [M+H].

### Example 74

### (S)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one

Compounds 74 was prepared following the procedure described as described in Example 64.

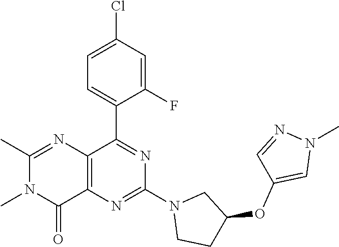

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

¹H-NMR (400 MHz, DMSO-d6): δ 7.67-7.56 (m, 2H), 7.52 (s, 1H), 7.45 (d, J=8.4 Hz, 1H), 7.21 (s, 1H), 4.79 (s, 1H), 3.90-3.67 (m, 6H), 3.65-3.57 (m, 1H), 3.50 (s, 3H), 2.42 (s, 3H), 2.23 (s, 2H).

LCMS Condition H: Rt=2.90 min. m/z 470.3 [M+H]⁺.

### Example 75

### (S)-8-(4-chloro-2-fluorophenyl)-2,3-dimethyl-6-(3-((1-methyl-1H-pyrazol-4-yl)oxy)piperidin-1-yl)pyrimido[5,4-d]pyrimidin-4 (3H)-one

Compounds 75 was prepared following the procedure described as described in Example 64.

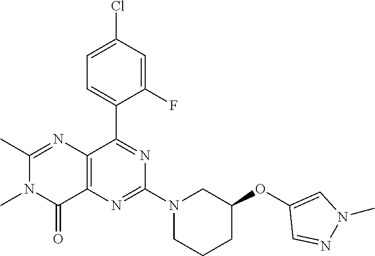

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

1H NMR (400 MHz, DMSO-d6, 100° C.) δ 7.62 (t, J=7.9 Hz, 1H), 7.54 (s, 1H), 7.48 (d, J=10.1 Hz, 1H), 7.41 (d, J=8.3 Hz, 1H), 7.14 (s, 1H), 4.40-4.32 (m, 1H), 4.13-3.94 (m, 2H), 3.79-3.62 (m, 2H), 3.74 (s, 3H), 3.54 (s, 3H), 2.45 (s, 3H), 2.06 (m, 1H), 1.84-1.78 (m, 2H), 1.57-1.56 (m, 1H).

LCMS Condition H: Rt=2.97 min. m/z 484.4 [M+H]⁺.

### Example 76

### (R)-8-(4-chloro-2-fluorophenyl)-2,3-dimethyl-6-(3-((1-methyl-1H-pyrazol-4-yl)oxy)piperidin-1-yl)pyrimido[5,4-d]pyrimidin-4 (3H)-one

Compounds 76 was prepared following the procedure described as described in Example 64.

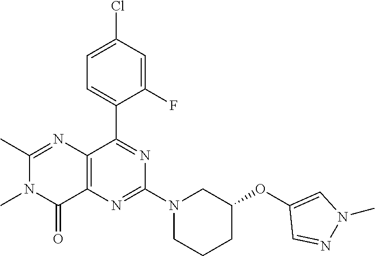

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

1H NMR (400 MHz, DMSO-d6, 100° C.) δ 7.62 (t, J=7.9 Hz, 1H), 7.57-7.45 (m, 2H), 7.41 (d, J=8.2 Hz, 1H), 7.15 (s, 1H), 4.36 (d, J=12.8 Hz, 1H), 4.14-3.92 (m, 2H), 3.78-3.63 (m, 5H), 3.54 (s, 3H), 2.45 (s, 3H), 2.09-2.02 (m, 1H), 1.89-1.76 (m, 2H), 1.61-1.54 (m, 1H).

LCMS Condition H: Rt=2.98 min. m/z 484.4 [M+H]⁺.

### Example 77

### 8-(4-chloro-2-fluorophenyl)-2,3-dimethyl-6-(4-((1-methyl-1H-pyrazol-4-yl)oxy)piperidin-1-yl)pyrimido[5,4-d]pyrimidin-4 (3H)-one

Compounds 77 was prepared following the procedure described as described in Example 64 as follow.

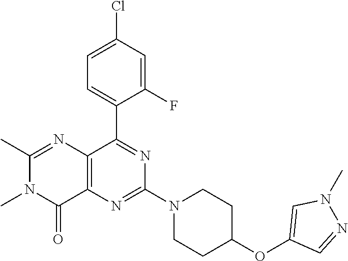

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

1H NMR (400 MHz, DMSO-d6) δ 7.70-7.56 (m, 2H), 7.52-7.42 (m, 2H), 7.21 (s, 1H), 4.30-4.17 (m, 3H), 3.73 (s, 3H), 3.65-3.54 (m, 2H), 3.51 (s, 3H), 2.43 (s, 3H), 2.02-1.97 (m, 2H), 1.64-1.59 (m, 2H).

LCMS Condition P: Rt=2.72 min. m/z 484.1 [M+H]⁺.

### Example 78

### 8-(4-chloro-2-fluorophenyl)-2,3-dimethyl-6-(3-morpholinopiperidin-1-yl)pyrimido[5,4-d]pyrimidin-4 (3H)-one

### Step-1—Preparation of tert-butyl 3-morpholinopiperidine-1-carboxylate

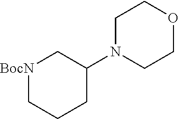 [SMILES: CC(C)(C)OC(=O)N1CCCC(N2CCOCC2)C1]

To a stirred solution of tert-butyl 3-oxopiperidine-1-carboxylate (500 mg, 2.509 mmol) and morpholine (328 mg, 3.764 mmol) in DCE (10 ml), was added acetic acid (0.14 ml) and NaBH(OAc)3 (1.6 g, 7.53 mmol) at rt. Then the reaction mixture was stirred for 16 h at rt. After completion of the reaction, reaction mixture was quenched with 1 N NaOH solution and extracted with DCM. Organic layer was dried over anhydrous sodium sulfate. Filtrate was concentrated under reduced pressure to afford the crude which was purified through column chromatography using 100-200 mesh silica gel with 45%. ethyl acetate in hexane to afford tert-butyl 3-morpholinopiperidine-1-carboxylate (280 mg, 41%) as colorless liquid.

LCMS Condition of 2: Rt=3.09 min. m/z 270.9 [M+H]+

### Step-2—Preparation of 4-(piperidin-3-yl)morpholine (HCl salt)

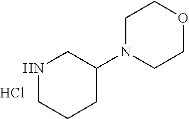 [SMILES: Cl ; C1CNCC(N2CCOCC2)C1]

To a stirred solution of tert-butyl 3-morpholinopiperidine-1-carboxylate (280 mg, 1.036 mmol) in DCM (3 ml) was added 4M HCl in dioxane (1 ml). Then it was stirred at room temperature for 3 h. After completion of the reaction, it was concentrated under reduced pressure and dried under vacuum to afford 4-(piperidin-3-yl)morpholine (150 mg, HCl salt, 85%) as sticky solid.

LCMS Condition of 3: Rt=0.61 min. m/z 171.0 [M+H]+

### Step-3—Preparation of 8-(4-chloro-2-fluorophenyl)-2,3-dimethyl-6-(3-morpholinopiperidin-1-yl)pyrimido[5,4-d]pyrimidin-4 (3H)-one

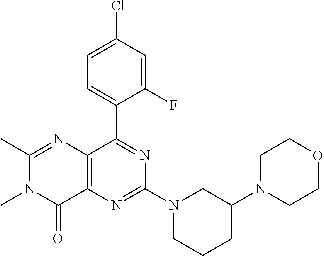 [SMILES: Cc1nc2c(-c3ccc(Cl)cc3F)nc(N3CCCC(N4CCOCC4)C3)nc2c(=O)n1C]

Step-3 was done following the protocol as described in example 48.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

1H NMR (400 MHz, DMSO-d6) δ 7.68-7.57 (m, 2H), 7.46 (dd, J=1.7, 8.3 Hz, 1H), 4.65-4.60 (m, 1H), 4.47 (d, J=13.2 Hz, 1H), 3.58-3.51 (m, 4H), 3.50 (s, 3H), 3.19-3.09 (m, 2H), 2.64-2.50 (m, 4H), 2.42 (s, 3H), 2.35-2.24 (m, 1H), 1.96-1.88 (m, 1H), 1.83-1.75 (m, 1H), 1.59-1.48 (m, 1H), 1.48-1.37 (m, 1H).

LCMS Condition P: Rt=2.70 min. m/z 473.1 [M+H]⁺.

### Example 79

### 8-(4-chloro-2-fluorophenyl)-6-(3-(3-methoxyazetidin-1-yl)piperidin-1-yl)-2,3-dimethylpyrimido[5,4-d]pyrimidin-4 (3H)-one

Compounds 79 was prepared following the procedure described as described in Example 78 as follow.

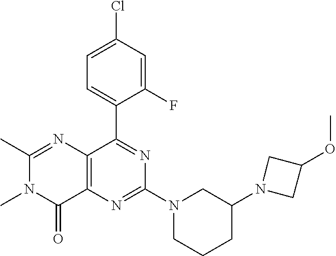

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

1H NMR (400 MHz, DMSO-d6) δ 7.68-7.56 (m, 2H), 7.46 (dd, J=1.8, 8.3 Hz, 1H), 4.04-3.84 (m, 3H), 3.68-3.60 (m, 1H), 3.50 (s, 4H), 3.47-3.40 (m, 2H), 3.12 (s, 3H), 2.82-2.76 (m, 2H), 2.42 (s, 3H), 2.18 (s, 1H), 1.75-1.70 (m, 2H), 1.41-1.35 (m, 2H).

LCMS Condition P: Rt=2.64 min. m/z 473.2 [M+H]⁺.

### Example 80

### 8-(4-chloro-2-fluorophenyl)-6-(3-(3,3-difluoroazetidin-1-yl)piperidin-1-yl)-2,3-dimethylpyrimido[5,4-d]pyrimidin-4 (3H)-one

Compounds 80 was prepared following the procedure described as described in Example 78 as follow.

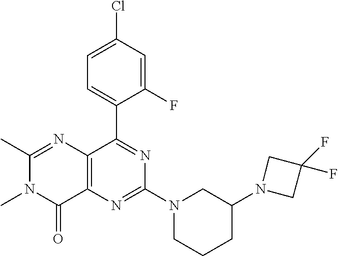 [SMILES: Cc1nc2c(-c3ccc(Cl)cc3F)nc(N3CCCC(N4CC(F)(F)C4)C3)nc2c(=O)n1C]

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 20% A and 80% B in 20 min, then to 5% A and 95% B in 21 min., held this composition up to 22 min. for column washing, then returned to initial composition in 23 min. and held till 25 min.

1H NMR (400 MHz, DMSO-d6) δ 7.69-7.57 (m, 2H), 7.46 (dd, J=1.8, 8.3 Hz, 1H), 3.94-3.77 (m, 3H), 3.70-3.44 (m, 8H), 2.42 (s, 4H), 1.80-1.68 (m, 2H), 1.46-1.41 (m, 2H).

LCMS Condition H: Rt=2.66 min. m/z 479.4 [M+H]⁺.

### Synthesis of Example 81 to 86

Compounds 81 to 86 were synthesized following the procedure as described in Example 48.

Example 81-86 were purified by Prep-HPLC using following method:

Preparative HPLC was done on Waters auto purification instrument. Column name: YMC-Actus 018 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 50% A and 50% B in 3 min, then to 20% A and 80% B in 20 min., then to 5% A and 95% B in 21 min., held this composition up to 22 min. for column washing, then returned to initial composition in 23 min. and held till 25 min.

| | | | |
|---|---|---|---|
| Ex. # | Structure | Name | Reagents/conditions |

| | | |
|---|---|---|
| Ex. 81 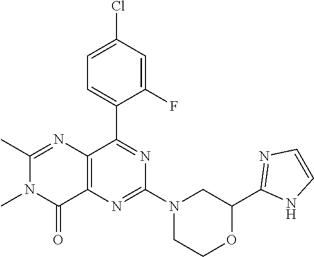 [SMILES: Cc1nc2c(-c3ccc(Cl)cc3F)nc(N3CCOC(c4ncc[nH]4)C3)nc2c(=O)n1C] | 6-(2-(1H-imidazol-2- yl)morpholino)-8-(4-chloro-2- fluorophenyl)-2,3- dimethylpyrimido[5,4-d]pyrimidin- 4(3H)-one | Step-1 2-(1H-imidazol- 2-yl)morpholine |
| Ex. 82 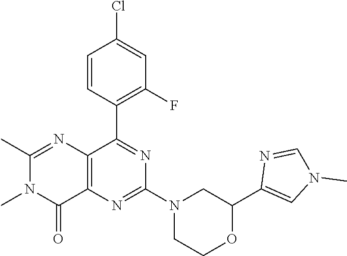 | 8-(4-chloro-2-fluorophenyl)-2,3- dimethyl-6-(2-(1-methyl-1H- imidazol-4- yl)morpholino)pyrimido[5,4- d]pyrimidin-4(3H)-one | Step-1 2-(1-methyl-1H- imidazol-4- yl)morpholine |
| Ex. 83 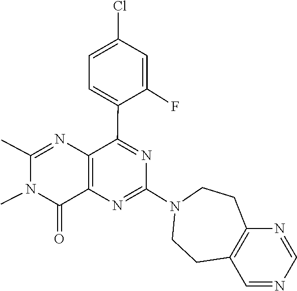 [SMILES: Cc1nc2c(-c3ccc(Cl)cc3F)nc(N3CCc4cncnc4CC3)nc2c(=O)n1C] | 8-(4-chloro-2-fluorophenyl)-2,3- dimethyl-6-(5,6,8,9-tetrahydro-7H- pyrimido[4,5-d]azepin-7- yl)pyrimido[5,4-d]pyrimidin-4(3H)- one | Step-1 6,7,8,9- tetrahydro-5H- pyrimido[4,5- d]azepine |
| Ex.84 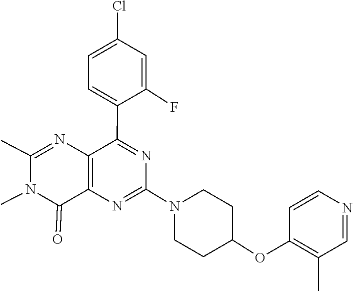 [SMILES: Cc1cnccc1OC1CCN(c2nc(-c3ccc(Cl)cc3F)c3nc(C)n(C)c(=O)c3n2)CC1] | 8-(4-chloro-2-fluorophenyl)-2,3- dimethyl-6-(4-((3-methylpyridin-4- yl)oxy)piperidin-1-yl)pyrimido [5,4-d]pyrimidin-4(3H)-one | Step-1 3-methyl-4- (piperidin-4- yloxy)pyridine |
| Ex. 85 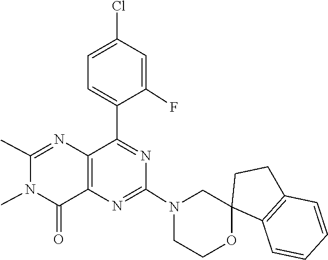 [SMILES: Cc1nc2c(-c3ccc(Cl)cc3F)nc(N3CCOC4(CCc5ccccc54)C3)nc2c(=O)n1C] | 8-(4-chloro-2-fluorophenyl)-6-(2,3- dihydrospiro[indene-1,2′- morpholin]-4′-yl)-2,3- dimethylpyrimido[5,4-d]pyrimidin- 4(3H)-one | Step-1 2,3- dihydrospiro [indene-1,2′- morpholine] |
| Ex. 86 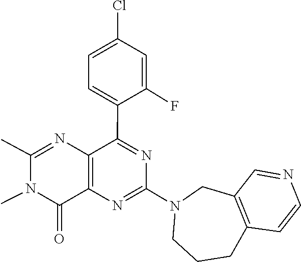 [SMILES: Cc1nc2c(-c3ccc(Cl)cc3F)nc(N3CCCc4ccncc4C3)nc2c(=O)n1C] | 8-(4-chloro-2-fluorophenyl)-2,3- dimethyl-6-(5,6,7,9-tetrahydro-8H- pyrido[3,4-c]azepin-8- yl)pyrimido[5,4-d]pyrimidin- 4(3H)-one | Step-1 6,7,8,9- tetrahydro-5H- pyrido[3,4-c] azepine |

Analytical data

| Ex. # | 1H-NMR | LCMS | Description | Yield (%) |
|---|---|---|---|---|
| Ex. 81 | 1H NMR (400 MHz, DMSO-d6) δ 12.07 (s, | Condition P: | Yellow solid | 32.0 |
| | 1H), 7.68 (t, J = 7.9 Hz, 1H), 7.58 (d, J = | Rt = 2.48 | | |
| | 9.8 Hz, 1H), 7.43 (d, J = 8.3 Hz, 1H), 7.09 | min. m/z | | |
| | (s, 1H), 6.87 (s, 1H), 4.78 (d, J = 13.1 Hz, | 456.1 | | |
| | 1H), 4.64-4.52 (m, 2H), 4.05 (d, J = 10.1 | [M + H]⁺. | | |
| | Hz, 1H), 3.75-3.64 (m, 1H), 3.49 (s, 3H), | | | |
| | 3.35-3.24 (m, 1H), 3.25-3.12 (m, 1H), | | | |
| | 2.41 (s, 3H). | | | |
| Ex. 82 | 1H NMR (400 MHz, DMSO) δ 7.70-7.57 | Condition H: | Yellow solid | 54.9 |
| | (m, 2H), 7.53 (s, 1H), 7.46 (d, J = 8.3 Hz, | Rt = 2.42 | | |
| | 1H), 7.10 (s, 1H), 4.77 (d, J = 13.5 Hz, 1H), | min. m/z | | |
| | 4.54 (d, J = 12.9 Hz, 1H), 4.47-4.40 (m, | 470.3 | | |
| | 1H), 4.01 (d, J = 10.8 Hz, 1H), 3.72-3.65 | [M + H]⁺. | | |
| | (m, 1H), 3.63 (s, 3H), 3.51 (s, 3H), 3.17- | | | |
| | 3.12 (m, 2H), 2.44 (s, 3H). | | | |
| Ex. 83 | 1H-NMR (400 MHz, DMSO-d6): δ 8.89 (s, | Condition H: | Yellow solid | 56.1 |
| | 1H), 8.56 (s, 1H), 7.69 (t, J = 8.0 Hz, 1H), | Rt = 2.71 | | |
| | 7.61 (d, J = 9.6 Hz, 1H), 7.47 (d, J = 8.0 Hz, | min. m/z | | |
| | 1H), 4.11-4.07 (m, 4H), 3.51 (s, 3H), 3.17 | 453.2 | | |
| | (bs, 2H), 3.00 (bs, 2H), 2.43 (s, 3H). | [M + H]⁺. | | |
| Ex. 84 | 1H-NMR (400 MHz, DMSO-d6): δ 8.27 (d, | Condition H: | Yellow solid | 56.9 |
| | J = 5.6 Hz, 1H), 8.23 (s, 1H), 7.68-7.59 (m, | Rt = 2.51 | | |
| | 2H), 7.47-7.44(m, 1H), 7.09 (t, J = 8.0 Hz, | min. m/z | | |
| | 1H), 4.88 (t, J = 5.6 Hz, 1H), 4.17-4.13 (m, | 495.3 | | |
| | 2H), 3.84-3.79 (m, 2H), 3.51(s, 3H), 2.43 | [M + H]⁺. | | |
| | (s, 3H), 2.12 (s, 3H), 2.07-2.03 (m, 2H), | | | |
| | 1.75-1.72 (m, 2H). | | | |
| Ex. 85 | 1H-NMR (400 MHz, DMSO-d6): δ 7.66- | Condition p: | Yellow solid | 37.2 |
| | 7.58 (m, 2H), 7.43 (d, J = 8.4 Hz, 1H), 7.38 | Rt = 3.19 | | |
| | (d, J = 7.2 Hz, 1H), 7.28 (d, J = 4.0 Hz, 2H), | min. m/z | | |
| | 7.23-7.19 (m, 1H), 4.30 (d, J = 12.0 Hz, | 492.4 | | |
| | 1H), 4.20 (d, J = 13.2 Hz, 1H), 3.88-3.76 | [M + H]⁺. | | |
| | (m, 2H), 3.63-3.55 (m, 2H), 3.50 (s, 3H), | | | |
| | 3.00-2.93 (m, 1H), 2.84-2.76 (m, 1H), 2.43 | | | |
| | (s, 3H), 2.31-2.24 (m, 1H), 2.07-2.00 (m, 1H). | | | |
| Ex. 86 | 1H-NMR (400 MHz, DMSO-d6, 100 oC): δ | Condition H: | Yellow solid | 70.3 |
| | 8.33-8.31 (m, 2H), 761 (t, J = 8.0 Hz, 1H), | Rt = 2.44 | | |
| | 7.50 (d, J = 7.6 Hz, 1H), 7.43 (d, J = 8.4 Hz, | min. m/z | | |
| | 1H), 7.35 (s, 1H), 4.91 (s, 2H), 4.22 (s, 2H), | 451.4 | | |
| | 3.50 (s, 3H), 2.41 (s, 3H), 1.84 (bs, 2H), | [M + H]⁺. | | |
| | 1.26 (bs, 2H). | | | |

### Example 87

### 2,3-dimethyl-6-(2-(1-methyl-1H-pyrazol-4-yl)morpholino)-8-(piperidin-4-yl)pyrimido[5,4-d]pyrimidin-4 (3H)-one

### Step-1—Preparation of tert-butyl 4-(2-chloro-6,7-dimethyl-8-oxo-7,8-dihydropyrimido[5,4-d]pyrimidin-4-yl)-3,6-dihydropyridine-1 (2H)-carboxylate

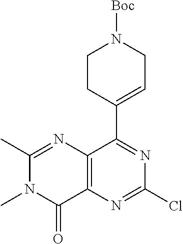 [SMILES: Cc1nc2c(C3=CCN(C(=O)OC(C)(C)C)CC3)nc(Cl)nc2c(=O)n1C]

To a stirred solution of 6,8-dichloro-2,3-dimethylpyrimido[5,4-d]pyrimidin-4 (3H)-one (300 mg, 1.22 mmol) and tert-butyl4-(4,4,5,5-tetramethyl-1,3,2-dioxaborolan-2-yl)-3,6-dihydropyridine-1 (2H)-carboxylate (378.53 mg, 1.22 mmol) in dioxane (8 mL) and water (2 mL) was added sodium carbonate (194.62 mg, 1.83 mmol) and degassed with argon. PdCl₂(dppf) (89.26 mg, 0.122 mmol) was added under inert atmosphere. Resulting mixture was heated at 80° C. for 5 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (10-50) % ethyl acetate in hexane as eluent to afford 6-chloro-8-(4-chloro-2-fluorophenyl)-2,3-dimethylpyrimido[5,4-d]pyrimidin-4 (3H)-one (300 mg, 62.52% yield).

LCMS Condition B: Rt=1.84 min. m/z 392.1 [M+H]⁺.

### Step-2—Preparation of tert-butyl 4-(6,7-dimethyl-2-(2-(1-methyl-1H-pyrazol-4-yl)morpholino)-8-oxo-7,8-dihydropyrimido[5,4-d]pyrimidin-4-yl)-3,6-dihydropyridine-1 (2H)-carboxylate

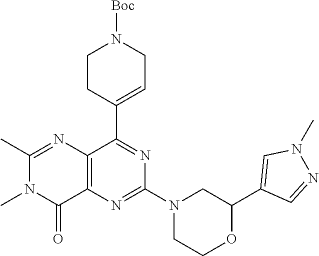

To a suspension of tert-butyl 4-(2-chloro-6,7-dimethyl-8-oxo-7,8-dihydropyrimido[5,4-d]pyrimidin-4-yl)-3,6-dihydropyridine-1 (2H)-carboxylate (260.0 mg, 0.662 mmol) and 2-(1-methyl-1H-pyrazol-4-yl)morpholine (221.5 mg, 1.325 mmol; HCl salt) in 1,4-dioxane (6 mL) was added DIPEA (0.346 mL, 2.0 mmol) and heated the reaction mixture at 40° C. for 10 h. After completion, excess solvent was removed under reduced pressure and residue was washed with brine and followed by extracted with EtOAc (5% MeOH). Combined organic layer was dried over sodium sulfate and then concentrated into vacuo. Purification was performed by column chromatography on silica gel (100-200 mesh size) using (0-5)% MeOH in EtOAc as eluent to afford tert-butyl 4-{6,7-dimethyl-2-[2-(1-methyl-1H-pyrazol-4-yl)morpholin-4-yl]-8-oxo-7H,8H-[1,3]diazino[5,4-d]pyrimidin-4-yl}-1,2,3,6-tetrahydropyridine-1-carboxylate (160.0 mg, 46.22% yield; ~40% by LC-MS; inseparable mixture) as yellow solid.

LCMS Condition B: Rt=1.806 min. m/z 523.4 [M+H]⁺.

### Step-3—Preparation of tert-butyl 4-(6,7-dimethyl-2-(2-(1-methyl-1H-pyrazol-4-yl)morpholino)-8-oxo-7,8-dihydropyrimido[5,4-d]pyrimidin-4-yl)piperidine-1-carboxylate

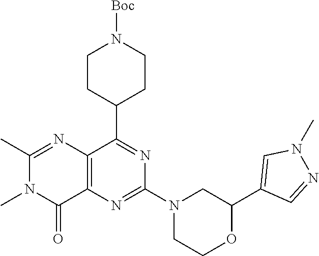

A solution of tert-butyl 4-(6,7-dimethyl-2-(2-(1-methyl-1H-pyrazol-4-yl)morpholino)-8-oxo-7,8-dihydropyrimido[5,4-d]pyrimidin-4-yl)-3,6-dihydropyridine-1 (2H)-carboxylate (140.0 mg, 0.268 mmol) in dry EtOH (10 mL, containing 10% THF) was purged with argon and followed by Pd—C (70.0 mg; 10% on activated C) was added to it. Reaction mixture was hydrogenated using hydrogen balloon for 8 h. After completion, reaction mixture was passed through short cartage filter and washed with EtOAc. Evaporation of the solvent under reduced pressure afforded title compound tert-butyl 4-{6,7-dimethyl-2-[2-(1-methyl-1H-pyrazol-4-yl)morpholin-4-yl]-8-oxo-7H,8H-[1,3]diazino[5,4-d]pyrimidin-4-yl}piperidine-1-carboxylate (100.0 mg, 71.14% yield; ~60% pure as per LC-MS) as yellow sticky solid. This material was forwarded for the next step without further purification.

LCMS Condition B: Rt=1.92 min. m/z 525.3 [M+H]⁺.

### Step-4—2,3-dimethyl-6-(2-(1-methyl-1H-pyrazol-4-yl)morpholino)-8-(piperidin-4-yl)pyrimido[5,4-d]pyrimidin-4 (3H)-one

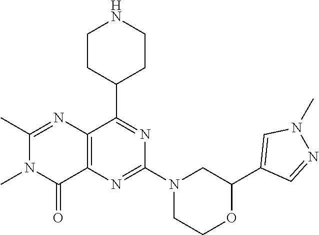

tert-butyl 4-(6,7-dimethyl-2-(2-(1-methyl-1H-pyrazol-4-yl)morpholino)-8-oxo-7,8-dihydropyrimido[5,4-d]pyrimidin-4-yl)piperidine-1-carboxylate (100.0 mg, 0.20 mmol) was dissolved in dry DCM (4 mL) and cooled the reaction mixture at 0° C. TFA (0.58 mL, 7.63 mmol) was slowly added to it and reaction mixture was stirred at room temperature until all reactant was consumed. After completion, excess volatiles were removed under reduced pressure and the residue was performed by prep-HPLC to obtain tert-butyl 4-(6,7-dimethyl-2-(2-(1-methyl-1H-pyrazol-4-yl)morpholino)-8-oxo-7,8-dihydropyrimido[5,4-d]pyrimidin-4-yl)piperidine-1-carboxylate (12.0 mg, 15.0% yield) as yellow semi-solid.

1H NMR (400 MHz, DMSO-d6) δ 8.53-7.97 (m, 1H), 7.76 (s, 1H), 7.47 (s, 1H), 4.62 (d, J=13.3 Hz, 1H), 4.56-4.45 (m, 2H), 4.05-3.97 (m, 1H), 3.94-3.84 (m, 1H), 3.83 (s, 3H), 3.70-3.59 (m, 2H), 3.51 (s, 3H), 3.35 (s, 1H), 3.19-3.07 (m, 2H), 3.09-3.04 (m, 2H), 2.55 (s, 3H), 1.96-1.91 (m, 4H).

LCMS Condition H: Rt=2.53 min. m/z 425.3 [M+H]⁺.

### Example 88A and Example 88B

### 2,3-dimethyl-6-((2S,4R)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one and 2,3-dimethyl-6-((2R,4S)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one

### Step-1—Preparation of 2,3-dimethyl-6-(6-(1-methyl-1H-pyrazol-4-yl)-3,6-dihydro-2H-pyran-4-yl)-8-(6-(trifluoromethyl)pyridin-3-yl)pyrimido[5,4-d]pyrimidin-4 (3H)-one

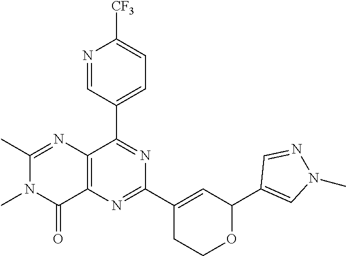

To a suspension of 6-chloro-2,3-dimethyl-8-(6-(trifluoromethyl)pyridin-3-yl)pyrimido[5,4-d]pyrimidin-4 (3H)-one (400.0 mg, 1.127 mmol) and -methyl-4-(4-(4,4,5,5-tetramethyl-1,3,2-dioxaborolan-2-yl)-5,6-dihydro-2H-pyran-2-yl)-1H-pyrazole (524.0 mg, 1.7 mmol) in a mixture of 1,4-dioxane/water (10 mL; 8:2; v/v) was added cesium carbonate (734.14 mg, 2.253 mmol) and purged the reaction mixture with argon atmosphere for 5 min. PdCl₂(dppf)DCM complex (92.0 mg, 0.113 mmol) was added to it under inert atmosphere and finally it was heated for 3 h at 80° C. After completion, reaction mixture was passed through celite and washed with EtOAc. Concentration of the solvent afforded crude which was washed with water and followed by extracted with DCM. Combined organic layer was dried over sodium sulphate and concentrated into vacuo. Purification was performed by column chromatography on silica gel (100-200 mesh size) using (0-6) % MeOH in EtOAc as eluent to afford 2,3-dimethyl-6-(6-(1-methyl-1H-pyrazol-4-yl)-3,6-dihydro-2H-pyran-4-yl)-8-(6-(trifluoromethyl)pyridin-3-yl)pyrimido[5,4-d]pyrimidin-4 (3H)-one (300.0 mg, 55.08% yield) as yellow solid.

1H NMR (400 MHz, DMSO D6) δ 9.67 (s, 1H), 8.98 (d, J=8.0 Hz, 1H), 8.12 (d, J=8.2 Hz, 1H), 7.69 (s, 1H), 7.42 (s, 2H), 5.44 (s, 1H), 3.99-3.69 (m, 1H), 3.86-3.84 (m, 1H), 3.82 (s, 3H), 3.59 (s, 3H), 2.74 (bs, 2H), 2.64 (s, 3H).

LCMS Condition A: Rt=1.786 min. m/z 484.1 [M+H]⁺.

### Step-2—Preparation of 2,3-dimethyl-6-(2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one

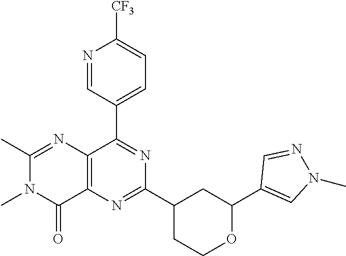

To a solution of 2,3-dimethyl-6-(6-(1-methyl-1H-pyrazol-4-yl)-3,6-dihydro-2H-pyran-4-yl)-8-(6-(trifluoromethyl)pyridin-3-yl)pyrimido[5,4-d]pyrimidin-4 (3H)-one (300.0 mg, 0.621 mmol) in 1,4-dioxane (25 mL) were added sodium acetate (102.0 mg, 1.24 mmol) and acetic acid (0.071 mL, 1.24 mmol) and purged the reaction mixture with argon. Pd(OH)2 (200.0 mg, 1.88 mmol; 20% wt.) was added to it and mixture was hydrogenated under balloon pressure until all the reactant was consumed. Progress of the reaction was monitored by LC-MS analysis. After completion, reaction mixture was passed through celite, washed with ethyl acetate and filtrate was concentrated under reduced pressure. Crude product was diluted with DCM and washed sequentially with saturated aq. NaHCO₃, water and brine. Organic layer was dried over Na2SO4, filtered and concentrated under reduced pressure. Crude product was purified by reverse phase prep-HPLC to get 2,3-dimethyl-6-(2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one (140 mg, 46.47% yield; mixture of diastereomers) as white solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 50% A and 50% B in 3 min, then to 10% A and 90% B in 20 min., then to 5% A and 95% B in 21 min., held this composition up to 22 min. for column washing, then returned to initial composition in 23 min. and held till 25 min.

1H NMR (400 MHz, DMSO D6) δ 9.65 (s, 1H), 8.94 (d, J=7.88 Hz, 1H), 8.13 (d, J=8.24 Hz, 1H), 7.65 (s, 1H), 7.38 (s, 1H), 4.53 (d, J=10.84 Hz, 1H), 4.12-4.09 (m, 1H), 3.78 (s, 3H), 3.72 (t, J=10.24 Hz, 1H), 3.46-3.43 (m, 1H), 2.63 (s, 3H), 2.18-2.24 (m, 1H), 2.04-1.98 (m, 1H), 1.96-1.90 (m, 2H).

LCMS Condition B: Rt=3.11 min. m/z 486.4 [M+H]⁺.

### Step-3—Preparation of 2,3-dimethyl-6-((2S,4R)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one [Example 88A] and 2,3-dimethyl-6-((2R,4S)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one [Example 88B]

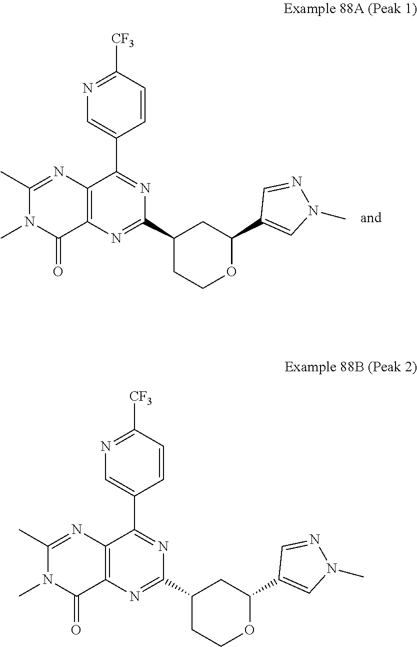

Chiral separation of 2,3-dimethyl-6-(2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one (140 mg, 0.064 mmol) was done by normal phase chiral prep HPLC to afford Example 88A, 2,3-dimethyl-6-((2S,4R)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one (45 mg; eluted first, assigned as Peak 1 with arbitrary assignment of stereochemistry) as light yellow solid and Example 88B 2,3-dimethyl-6-((2R,4S)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one (53 mg, eluted later, assigned as Peak 2 with arbitrary assignment of stereochemistry) as light yellow solid.

Chiral prep HPLC method details to separate the enantiomers:

Instrument: Agilent 1200 series instrument/Column: CHIRALPAK IC (250×21 mm) 5u/Flow rate: 21.0 ml/min./Mobile phase: Hexane/Dichloromethane/Ethanol: 65/17.5/17.5/Solubility: Mixture of Acetonitrile and Dichloromethane/Wave length: 295 nm/Run time: 36 min

Example 88A (Peak 1): 1H NMR (400 MHz, DMSO-d6) δ 9.65 (s, 1H), 8.95 (d, J=8.2 Hz, 1H), 8.14 (d, J=8.2 Hz, 1H), 7.65 (s, 1H), 7.38 (s, 1H), 4.53 (d, J=11.1 Hz, 1H), 4.11 (dd, J=4.4, 11.4 Hz, 1H), 3.86-3.66 (m, 4H), 3.59 (s, 3H), 3.52-3.41 (m, 1H), 3.38-3.28 (m, 1H), 2.64 (s, 3H), 2.31-2.22 (m, 1H), 2.10-1.83 (m, 2H).

LCMS Condition H: Rt=3.04 min. m/z 486.4 [M+H]⁺.

Example 88B (Peak 2): 1H NMR (400 MHz, DMSO-d6) δ 9.65 (s, 1H), 8.95 (d, J=8.4 Hz, 1H), 8.14 (d, J=8.2 Hz, 1H), 7.65 (s, 1H), 7.38 (s, 1H), 4.53 (d, J=10.4 Hz, 1H), 4.11 (dd, J=3.9, 10.9 Hz, 1H), 3.78 (s, 3H), 3.59 (s, 3H), 3.52-3.38 (m, 1H), 2.64 (s, 3H), 2.31-2.22 (m, 1H), 2.10-1.84 (m, 4H).

LCMS Condition H: Rt=3.03 min, m/z 486.4 [M+H]⁺.

Analytical chiral HPLS: Chiralpak IC (4.6×250 mm), 5μ Mobile Phase: Hexane/DCM/EtOH/IPAmine: 60/20/20/0.1 Flow Rate: 1.0 ml/min. Solubility: MeOH, Rt=4.93 min for Peak 1 and Rt=6.26 min for Peak 2.

### Example 89A and Example 89B

### (R)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one and (S)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one

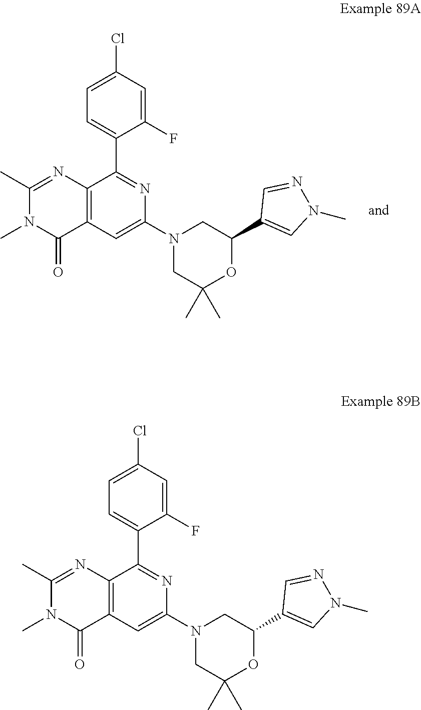

Chiral separation of 8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one (Example 62, 120 mg, 0.064 mmol) was done by normal phase chiral prep HPLC to afford Example 89A, (R)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one (35 mg, 29.6%; eluted first, assigned as Peak 1 with arbitrary assignment of stereochemistry) as yellow solid and Example 89B, (S)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one (36 mg, 29% yield, eluted later, assigned as Peak 2 with arbitrary assignment of stereochemistry) as yellow solid.

Chiral prep HPLC method details to separate the enantiomers: Instrument: Agilent 1200 series instrument/Column: CHIRALPAK IG (250×21 mm) 5u/Flow rate: 21.0 ml/min./Mobile phase: Hexane/Dichloromethane/Ethanol: 50/25/25/Solubility: Mixture of Acetonitrile and Dichloromethane/Wave length: 295 nm/Run time: 36 min

Example 89A (Peak 1): 1H NMR (400 MHz, DMSO-d6) δ 7.72 (s, 1H), 7.75-7.57 (m, 2H), 7.49-7.41 (m, 2H), 4.83-4.68 (m, 2H), 4.53 (d, J=12.6 Hz, 1H), 3.81 (s, 3H), 3.50 (s, 3H), 3.03-2.88 (m, 2H), 2.43 (s, 3H), 1.27-1.21 (m, 6H).

LCMS Condition H: Rt=2.40 min. m/z 498.4 [M+H]⁺.

Example 89B (Peak 2) 1H NMR (400 MHz, DMSO-d6) δ 7.75-7.57 (m, 3H), 7.49-7.41 (m, 2H), 4.83-4.68 (m, 2H), 4.53 (d, J=13.0 Hz, 1H), 3.81 (s, 3H), 3.50 (s, 3H), 3.03-2.88 (m, 2H), 2.43 (s, 3H), 1.27-1.21 (m, 6H).

LCMS Condition H: Rt=2.40 min. m/z 498.4 [M+H]C.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=6.79 min for Peak 1 and Rt=10.21 min for Peak 2.

### Example 90

### 8-(4-chloro-2-fluorophenyl)-2,3-dimethyl-6-(6-(1-methyl-1H-pyrazol-4-yl)-5-oxa-8-azaspiro(3,5)nonan-8-yl)pyrimido(5,4-d)pyrimidin-4 (3H)-one

Compound 90 was prepared following the procedure described in Preparation 1 steps 2-5, and Step-6 as described in Example 43-58.

| Ex. #/Structure | Name | LCMS/NMR | Description | Yield (%) |
|---|---|---|---|---|
| Step-2 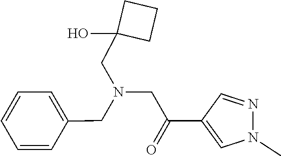 [SMILES: Cn1cc(C(=O)CN(Cc2ccccc2)CC2(O)CCC2)cn1] | 2-(benzyl(1- hydroxycyclobutyl) methyl)amino-1-(1- methyl-1-H-pyrazol- 4-yl)ethan-1-one | LCMS Condition A: Rt = 3.29 min. m/z 314.5 [M + H]⁺. | light gummy liquid | 91.0 |
| Step-3 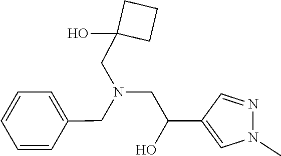 [SMILES: Cn1cc(C(O)CN(Cc2ccccc2)CC2(O)CCC2)cn1] | 2-(benzyl(2-hydroxy- 2-(1-methyl-1-H- pyrazol-4- yl)ethyl)amino)methyl) cyclobutan-1-ol | LCMS Condition A: Rt = 3.06 min. m/z 316.0 [M + H]⁺. | Gummy | 77.3 |
| Step-4 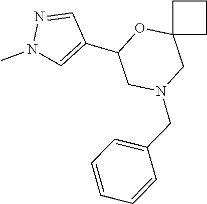 [SMILES: Cn1cc(C2CN(Cc3ccccc3)CC3(CCC3)O2)cn1] | 8-benzyl-6-(1- methyl-1H-pyrazol-4- yl)-5-oxa-8- azaspiro(3,5)- nonane (as HCl salt) | LCMS Condition A: Rt = 3.51 min. m/z 298.3 [M + H]⁺. | Yellow sticky solid | 90.0 |
| Step-5 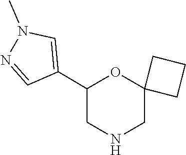 | 6-(1-methyl-1H- pyrazol-4-yl)-5-oxa- 8-azaspiro(3,5)- nonane (HCl salt) | LCMS Condition A: Rt = 1.27 min. m/z 208.2 [M + H]⁺. | sticky solid | 91.0 |
| Ex. 90 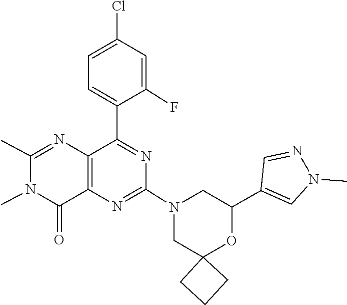 | 8-(4-chloro-2- fluorophenyl)-2,3- dimethyl-6-(6-(1- methyl-1H-pyrazol-4- yl)-5-oxa-8- azaspiro(3,5)nonan- 8-yl)pyrimido(5,4- d)pyrimidin-4(3H)- one | LCMS Condition H: Rt = 2.94 min. m/z 510.4 [M + H]⁺. | Yellow solid | 40.0 |

| Ex. # | 1H-NMR | LCMS | Description | Yield (%) |
|---|---|---|---|---|
| Ex. 90 | 1H NMR (400 MHz, DMSO) δ 7.75 | LCMS Condition | Yellow solid | 40.0 |
| | (s, 1H), 7.71-7.59 (m, 2H), 7.47 | H: Rt = 2.94 min. | | |
| | (d, J = 6.2 Hz, 2H), 4.81 (d, J = | m/z 510.4 [M + H]⁺. | | |
| | 13.1 Hz, 1H), 4.67-4.62 (m, 1H), | | | |
| | 4.53 (dd, J = 2.7, 11.0 Hz, 1H), | | | |
| | 3.81 (s, 3H), 3.51 (s, 3H), 3.08- | | | |
| | 2.92 (m, 2H), 2.44 (s, 3H), 2.07- | | | |
| | 1.94 (m, 4H), 1.80 (s, 1H), 1.70- | | | |
| | 1.63 (m, 1H). | | | |

### Example 91

### 8-(4-chloro-2-fluorophenyl)-2,3-dimethyl-6-(2-(2-methyl-2H-1,2,3-triazol-4-yl)morpholino)pyrimido[5,4-d]pyrimidin-4 (3H)-one

Example 91 was prepared using similar procedures to Example 2 and Example 35.

| | | | | | |
|---|---|---|---|---|---|
| Ex. # | Structure | 1H-NMR | LCMS | Description | Yield (%) |

| | | | | |
|---|---|---|---|---|
| Ex. 91 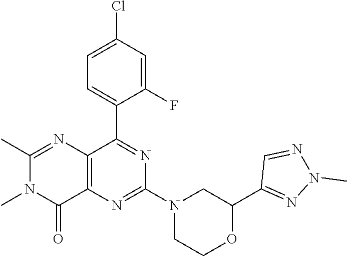 | 1H NMR (400 MHz, DMSO-d6) δ 7.77 (s, 1H), 7.71-7.57 (m, 2H), 7.46 (d, J = 8.2 Hz, 1H), 4.79 (d, J = 13.3 Hz, 1H), 4.73 (dd, J = 2.7, 10.4 Hz, 1H), 4.55 (d, J = 13.1 Hz, 1H), 4.15 (s, 3H), 4.06 (d, J = 10.2 Hz, 1H), 3.79-3.66 (m, 1H), 3.51 (s, 3H), 3.30- 3.16 (m, 2H), 2.44 (s, 3H). | LCMS Condition K: Rt = 2.81 min. m/z 471.3 [M + H]⁺. | Yellow solid | 39.4 |

### Example 92-Enantiomer 1

### (R)-6-(4,4-difluoro-3-(1-methyl-1H-pyrazol-4-yl)piperidin-1-yl)-2,3-dimethyl-8-(6-(trifluoromethyl)pyridin-3-yl)pyrimido[5,4-d]pyrimidin-4 (3H)-one

### Example 93—Enantiomer 2

### (S)-6-(4,4-difluoro-3-(1-methyl-1H-pyrazol-4-yl)piperidin-1-yl)-2,3-dimethyl-8-(6-(trifluoromethyl)pyridin-3-yl)pyrimido[5,4-d]pyrimidin-4 (3H)-one

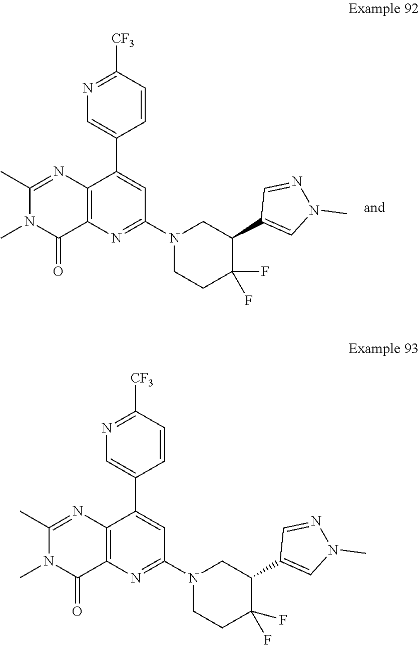

Synthesis of racemic 6-(4,4-difluoro-3-(1-methyl-1H-pyrazol-4-yl)piperidin-1-yl)-2,3-dimethyl-8-(6-(trifluoromethyl)pyridin-3-yl)pyrimido[5,4-d]pyrimidin-4 (3H)-one was done following procedure of Step-5, example-1 using 3a [6-chloro-2,3-dimethyl-8-(6-(trifluoromethyl)pyridin-3-yl)pyrimido[5,4-d]pyrimidin-4 (3H)-one] and 4,4-difluoro-3-(1-methyl-1H-pyrazol-4-yl)piperidine hydrochloride salt [step-7, Example-13] as starting materials. Chiral separation of racemic compound afforded Example 92 (Peak1, absolute stereochemistry unknown) as yellow solid in 35.2% yield and 93 (Peak2) as yellow solid in 30.3% yield, absolute stereochemistry unknown.

Chiral separation was done by SFC using PIC-SOLUTION-175 instrument by using I-CELLULOSE-Z column (21.1 mm×250 mm), 5p operating at 35° C. temperature, maintaining flow rate of 50 ml/min, using 65% CO2 in super critical state & 35% of (100% IPA) as mobile phase, Run this isocratic mixture upto 11 minutes and also maintained the isobaric condition of 100 bar at 297 nm wavelength.

Example 92 (Peak 1): 1H NMR (400 MHz, Chloroform-d) δ 9.68 (s, 1H), 8.83 (d, J=8.2 Hz, 1H), 7.84 (d, J=8.1 Hz, 1H), 7.52 (s, 1H), 7.42 (s, 1H), 4.93 (s, 2H), 3.93 (s, 3H), 3.67 (s, 5H), 3.36-3.14 (m, 1H), 2.62 (s, 3H), 2.34-2.23 (m, 1H), 2.17-1.98 (m, 1H).

LCMS Condition K: Rt=2.81 min. m/z 521.4 [M+H]⁺.

Example 93 (Peak 2) 1H NMR (400 MHz, Chloroform-d) δ 9.68 (s, 1H), 8.83 (d, J=8.1 Hz, 1H), 7.84 (d, J=8.0 Hz, 1H), 7.51 (s, 1H), 7.41 (s, 1H), 4.94 (s, 2H), 3.92 (s, 3H), 3.67 (s, 5H), 3.30-3.16 (m, 1H), 2.62 (s, 3H), 2.33-2.26 (m, 1H), 2.17-2.01 (m, 1H).

LCMS Condition K: Rt=2.81 min. m/z 521.4 [M+H]⁺.

Analytical chiral HPLC: Chiralpak IC (4.6×250 mm), 5μ Mobile Phase: Hexane/EA/EtOH/IPAmine: 70/15/15/0.1 Flow Rate: 1.0 ml/min. Solubility: MeOH, Rt=12.29 min for Peak 1 and Rt=13.21 min for Peak 2.

### Example 94-Diastereomer 1

### 8-(4-chloro-2-fluorophenyl)-2,3-dimethyl-6-((2R,4S)-2-(2-methylpyridin-4-yl)tetrahydro-2H-pyran-4-yl)pyrimido[5,4-d]pyrimidin-4 (3H)-one [cis-racemate]

### Example 95-Diastereomer 2

### 8-(4-chloro-2-fluorophenyl)-2,3-dimethyl-6-((2R,4R)-2-(2-methylpyridin-4-yl)tetrahydro-2H-pyran-4-yl)pyrimido[5,4-d]pyrimidin-4 (3H)-one [trans-racemate]

### Step-1—Preparation of 4-(4-bromotetrahydro-2H-pyran-2-yl)-2-methylpyridine

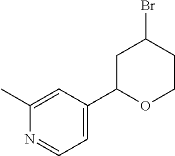

To a stirred solution of 2-methylisonicotinaldehyde (2.0 g, 16.51 mmol) and but-3-en-1-ol (1.399 mL, 16.51 mmol) in DCM (40.0 mL) was added a solution of HBr in acetic acid (33%, 10.0 mL) at 0° C. Resulting mixture was stirred at RT for 16 h. Reaction mixture was quenched using saturated aqueous NaHCO3 solution and extracted with DCM. The combined organic part was dried over anhydrous sodium sulphate, filtered and concentrated under reduced pressure. Crude mass was purified by column chromatography (30% ethyl acetate-hexane) to afford 4-(4-bromotetrahydro-2H-pyran-2-yl)-2-methylpyridine (2 g, 47.3% yield) as sticky liquid.

1H NMR (400 MHz, DMSO-d6) δ 8.38 (d, J=4.24 Hz, 1H), 7.21 (s, 1H), 7.12 (d, J=4.56 Hz, 1H), 4.90-4.75 (m, 1H), 4.56-4.54 (m, 1H), 3.97-3.91 (m, 2H), 2.44 (s, 3H), 2.23-2.14 (m, 2H), 2.03-1.82 (m, 2H)

LCMS Condition K: Rt=3.13 min. m/z 255.6 [M+H]⁺.

### Step-2—Preparation of 8-(4-chloro-2-fluorophenyl)-2,3-dimethyl-6-((2R,4S)-2-(2-methylpyridin-4-yl)tetrahydro-2H-pyran-4-yl)pyrimido[5,4-d]pyrimidin-4 (3H)-one [cis-racemate] and 8-(4-chloro-2-fluorophenyl)-2,3-dimethyl-6-((2R,4R)-2-(2-methylpyridin-4-yl)tetrahydro-2H-pyran-4-yl)pyrimido[5,4-d]pyrimidin-4 (3H)-one [trans-racemate]

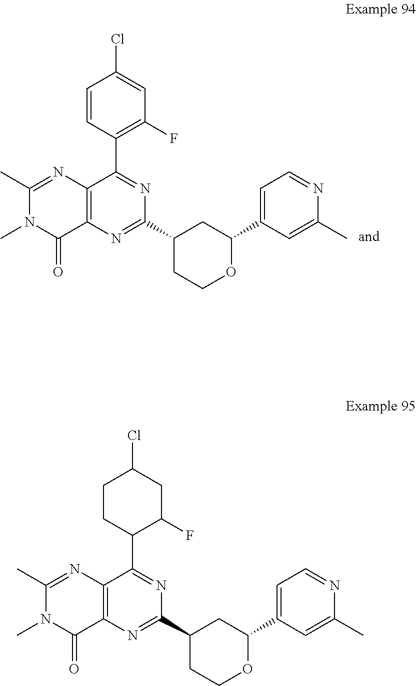

To a stirred solution of 6-chloro-8-(4-chloro-2-fluorophenyl)-2,3-dimethylpyrimido[5,4-d]pyrimidin-4 (3H)-one (200 mg, 0.592 mmol) and 4-(4-bromotetrahydro-2H-pyran-2-yl)-2-methylpyridine (226.3 mg, 0.888 mmol) in DME (6 ml) were added Lithium hydroxide (28.4 mg, 1.2 mmol) and 4,4′-Di-tert-butyl-2,2′-bipyridyl [CAS:72914-19-3](23.8 mg, 0.089 mmol) and degassed with argon. Nickel (II) chloride ethylene glycol dimethyl ether complex [CAS 29046-78-4](13.001 mg, 0.059 mmol) (4,4′-Di-tert-butyl-2,2′-bipyridine)bis[3,5-difluoro-2-[5-trifluoromethyl-2-pyridinyl-êN)phenyl-êC]iridium(II) hexafluorophosphate [CAS:870987-63-6](6.63 mg, 0.060 mmol) and tri(trimethylsilyl)silane (147.2 mg, 0.592 mmol) were added under inert condition. Resultant reaction mass was irradiated with 80 w blue LED light using as integrated photoreactor for 16 h. Reaction mass was concentrated. Crude product was purified by combiflash column chromatography eluted at (1-2% MeOH-DCM) as off-white solid [mixture of diastereomers].

Diastereomeric separation was done by reverse phase prep HPLC to afford 8-(4-chloro-2-fluorophenyl)-2,3-dimethyl-6-((2R,4S)-2-(2-methylpyridin-4-yl)tetrahydro-2H-pyran-4-yl)pyrimido[5,4-d]pyrimidin-4 (3H)-one as white solid (5 mg, 2% yield) and 8-(4-chloro-2-fluorophenyl)-2,3-dimethyl-6-((2R,4R)-2-(2-methylpyridin-4-yl)tetrahydro-2H-pyran-4-yl)pyrimido[5,4-d]pyrimidin-4 (3H)-one as white solid (8 mg, 4.5% yield).

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 55% A and 45% B in 3 min, then to 25% A and 75% B in 20 min., then to 5% A and 95% B in 21 min., held this composition up to 22 min. for column washing, then returned to initial composition in 23 min. and held till 25 min.

Example 94 (cis-racemate): 1H NMR (400 MHz, Chloroform-d) δ 8.47 (d, J=5.6 Hz, 1H), 7.60 (t, J=7.9 Hz, 1H), 7.46-7.33 (m, 2H), 7.30 (dd, J=1.8, 8.3 Hz, 1H), 7.23 (d, J=2.0 Hz, 1H), 4.58 (d, J=11.6 Hz, 1H), 4.39-4.30 (m, 1H), 3.85-3.74 (m, 1H), 3.67 (s, 3H), 3.66-3.56 (m, 1H), 2.73 (s, 3H), 2.58 (s, 3H), 2.37 (d, J=13.2 Hz, 1H), 2.23-2.11 (m, 1H), 2.02-1.87 (m, 2H).

LCMS Condition K: Rt=2.36 min. m/z 480.3 [M+H]⁺.

Example 95 (tans-racemate) 1H NMR (400 MHz, Chloroform-d) δ 8.47 (d, J=5.6 Hz, 1H), 7.67 (t, J=7.9 Hz, 1H), 7.39 (s, 1H), 7.36-7.24 (m, 3H), 4.97 (d, J=9.1 Hz, 1H), 4.05-3.97 (m, 1H), 3.86-3.71 (m, 2H), 3.69 (s, 3H), 2.79 (d, J=13.2 Hz, 1H), 2.70 (s, 3H), 2.61 (s, 3H), 2.47 (d, J=14.1 Hz, 1H), 2.30-2.20 (m, 1H), 2.07-2.00 (m, 1H).

LCMS Condition K: Rt=2.39 min. m/z 480.3 [M+H]⁺.

### Synthesis of Examples 96A, 96B to 98A, 98B

Examples 96A, 96B to 98A, 98B were synthesized following similar procedures as described in Example 14.

Chiral separation details of Examples 96A, 96B to 98A, 98B are captured in following table:

| | | | Chiral |
|---|---|---|---|
| Comp-ound No. and | | | separation |
| Structure | Name | Starting material | specification |
| Ex. 96A (Peak 1) 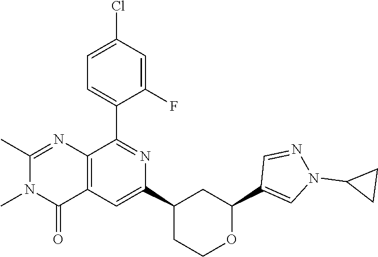 [SMILES: Cc1nc2c(-c3ccc(Cl)cc3F)nc([C@@H]3CCO[C@H](c4cnn(C5CC5)c4)C3)cc2c(=O)n1C] | 6-((2S,4R)-2-(1- cyclopropyl-1H- pyrazol-4- yl)tetrahydro- 2H-pyran-4-yl)- 8-(2-fluoro-4- (trifluoromethyl) phenyl)-2,3- dimethylpyrimido [5,4-d]pyrimidin- 4(3H)-one | 6-(2-(1-cyclopropyl-1H-pyrazol-4- yl)tetrahydro-2H-pyran-4-yl)-8-(2-fluoro- 4-(trifluoromethyl)phenyl)- 2,3-dimethylpyrimido[5,4-d]pyrimidin-4(3H)- one [cis racemate]: Synthesis following synthesized following procedure as described in Example 14 using intermediates 3 g and 1-cyclopropyl-4-(4-(4,4,5,5- tetramethyl-1,3,2-dioxaborolan-2-yl)-5,6- dihydro-2H-pyran-2-yl)-1H-pyrazole (product of Step-2, Example 14). | Similar specifications as in step-6 of example-1. Absolute stereochemistry unknown |
| Ex. 96B (Peak 2) 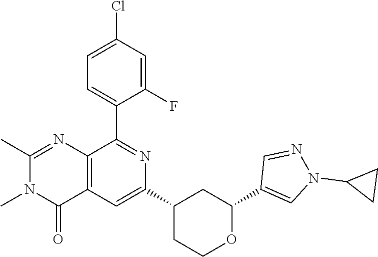 [SMILES: Cc1nc2c(-c3ccc(Cl)cc3F)nc([C@H]3CCO[C@@H](c4cnn(C5CC5)c4)C3)cc2c(=O)n1C] | 6-((2R,4S)-2-(1- cyclopropyl-1H- pyrazol-4- yl)tetrahydro- 2H-pyran-4-yl)- 8-(2-fluoro-4- (trifluoromethyl) phenyl)-2,3- dimethylpyrimido [5,4-d] pyrimidin- 4(3H)-one | | Absolute stereochemistry unknown |
| Ex. 97A (Peak 1) 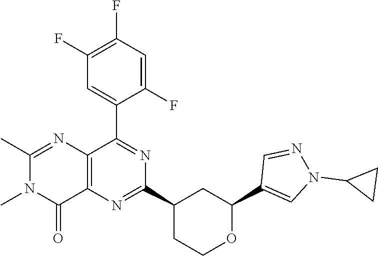 [SMILES: Cc1nc2c(-c3cc(F)c(F)cc3F)nc([C@@H]3CCO[C@H](c4cnn(C5CC5)c4)C3)nc2c(=O)n1C] | 6-((2S,4R)-2-(1- cyclopropyl-1H- pyrazol-4- yl)tetrahydro- 2H-pyran-4-yl)- 2,3-dimethyl-8- (2,4,5- trifluorophenyl) pyrimido[5,4-d] pyrimidin- 4(3H)-one | 6-(2-(1-cyclopropyl-1H-pyrazol-4- yl)tetrahydro-2H-pyran-4-yl)-2,3- dimethyl-8-(2,4,5-trifluorophenyl)pyrimido [5,4-d]pyrimidin-4(3H)-one [cis racemate]: Synthesis following synthesized following procedure as described in Example 14 using intermediates 3d and 1- cyclopropyl-4-(4-(4,4,5,5-tetramethyl- 1,3,2-dioxaborolan-2-yl)-5,6-dihydro-2H- pyran-2-yl)-1H-pyrazole (product of Step-2, Example 14). | Similar specifications as mentioned in step-6 of example-1. Absolute stereochemistry unknown |
| Ex. 97B (Peak 2) 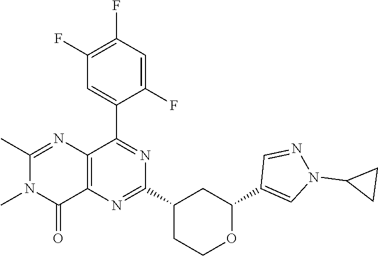 [SMILES: Cc1nc2c(-c3cc(F)c(F)cc3F)nc([C@H]3CCO[C@@H](c4cnn(C5CC5)c4)C3)nc2c(=O)n1C] | 6-((2R,4S)-2-(1- cyclopropyl-1H- pyrazol-4- yl)tetrahydro- 2H-pyran-4-yl)- 2,3-dimethyl-8- (2,4,5- trifluorophenyl) pyrimido[5,4- d]pyrimidin- 4(3H)-one | | Absolute stereochemistry unknown |
| Ex. 98A (Peak 1) 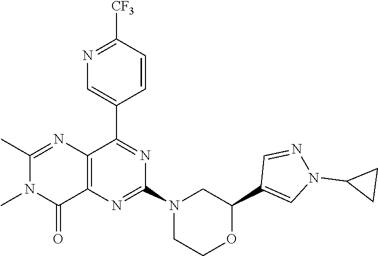 | 6-((2S,4R)-2-(1- cyclopropyl-1H- pyrazol-4- yl)tetrahydro- 2H-pyran-4-yl)- 2,3-dimethyl-8- (6- (trifluoromethyl) pyridin-3- yl)pyrimido [5,4-d]pyrimidin- 4(3H)-one | 6-(2-(1-cyclopropyl-1H-pyrazol-4-yl) tetrahydro-2H-pyran-4-yl)-2,3-dimethyl- 8-(6-(trifluoromethyl)pyridin-3-yl) pyrimido[5,4-d]pyrimidin-4(3H)-one [cis racemate]: Synthesis following synthesized following procedure as described in Example 14 using intermediates 3a and 1-cyclopropyl-4-(4- (4,4,5,5-tetramethyl-1,3,2-dioxaborolan- 2-yl)-5,6-dihydro-2H-pyran-2-yl)-1H- pyrazole (product of Step-2, Example 14). | Similar specifications as mentioned in step-6 of example Absolute stereochemistry unknown |
| Ex. 98B (Peak 2) 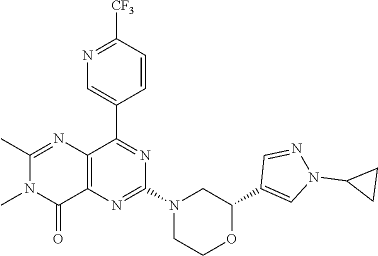 | 6-((2R,4S)-2-(1- cyclopropyl-1H- pyrazol-4- yl)tetrahydro- 2H-pyran-4-yl)- 2,3-dimethyl-8- (6- (trifluoromethyl) pyridin-3- yl)pyrimido[5,4- d]pyrimidin- 4(3H)-one | | Absolute stereochemistry unknown |

Following table describes analytical data analysis and yield information of Examples 96A, 96B to 98A, 98B:

| Compound | | | Analytical | | Yield |
|---|---|---|---|---|---|
| No. | HNMR | LCMS | chiral HPLC | Description | (%) |
| Ex. 96A | ¹H NMR (400 MHz, DMSO-d₆) δ | Condition | Chiralpak | Beige | 31.6 |
| (Peak 1) | 7.91-7.87 (m, 2H), 7.79 (d, J = | K: Rt = | IC (4.6 × | Solid | |
| | 8.1 Hz, 1H), 7.70 (s, 1H), 7.36 (s, | 2.89 min. | 250 mm), 5 μ | | |
| | 1H), 4.49 (d, J = 10.4 Hz, 1H), | m/z 529.4 | Mobile Phase: | | |
| | 4.09-4.06 (m, 1H), 3.71-3.62 | [M + H]⁺. | Hexane/DCM/ | | |
| | (m, 2H), 3.56 (s, 3H), 3.50-3.40 | | EtOH/IP | | |
| | (m, 1H), 2.60-2.50 (m, 3H), 2.22 | | Amine: | | |
| | (d, J = 12.7 Hz, 1H), 1.96-1.84 | | 60/20/20/0.1 | | |
| | (m, 3H), 1.0-0.87 (m, 4H). | | Flow Rate: | | |
| Ex. 96B | ¹H NMR (400 MHz, DMSO-d₆) δ | Condition | 1.0 ml/min | Beige | 32.4 |
| (Peak 2) | 7.91-7.89 (m, 2H), 7.79 (d, J = | K: Rt = | Solubility: | Solid | |
| | 8.1 Hz, 1H), 7.70 (s, 1H), 7.36 (s, | 2.89 min. | MeOH Rt = | | |
| | 1H), 4.49 (d, J = 11.1 Hz, 1H), | m/z 529.4 | 10.21 min | | |
| | 4.10-4.06 (m, 1H), 3.71-3.62 | [M + H]⁺. | for Peak 1 | | |
| | (m, 2H), 3.56 (s, 3H), 3.50-3.40 | | and Rt = | | |
| | (m, 1H), 2.60-2.50 (m, 3H), 2.22 | | 11.72 min | | |
| | (d, J = 13.4 Hz, 1H), 2.0-1.84 (m, | | for Peak 2. | | |
| | 3H), 1.0-0.90 (m, 4H). | | | | |
| Ex. 97A | ¹H NMR (400 MHz, DMSO-d₆) δ | Condition | Chiralpak | Off | 15.1 |
| (Peak 1) | 7.83-7.78 (m, 2H), 7.78 (s 1H), | K: Rt = | IC (4.6 × | white | |
| | 7.70 (s, 1H), 7.36 (s, 1H), 4.49 (d, | 2.75 min. | 250 mm), 5 μ | solid | |
| | J = 11.1 Hz, 1H), 4.10-4.06 (m, | m/z 497.4 | Mobile Phase: | | |
| | 1H), 3.71-3.62 (m, 2H), 3.56 (s, | [M + H]⁺. | Hexane/DCM/ | | |
| | 3H), 3.50-3.40 (m, 1H), 2.53 (s, | | EtOH/IP | | |
| | 3H), 2.22 (d, J = 13.4 Hz, 1H), | | Amine: | | |
| | 1.93-1.84 (m, 3H), 0.99-0.90 | | 50/25/25/0.1 | | |
| | (m, 4H). | | Flow Rate: | | |
| Ex. 97B | ¹H NMR (400 MHz, DMSO-d₆) δ | Condition | 1.0 ml/min | Off | 12.5 |
| (Peak 2) | 7.83-7.78 (m, 2H), 7.78 (s 1H), | K: Rt = | Solubility: | white | |
| | 7.70 (s, 1H), 7.36 (s, 1H), 4.49 (d, | 2.75 min. | MeOH Rt = | solid | |
| | J = 11.1 Hz, 1H), 4.10-4.06 (m, | m/z 497.4 | 8.45 min for | | |
| | 1H), 3.71-3.62 (m, 2H), 3.56 (s, | [M + H]⁺. | Peak 1 and | | |
| | 3H), 3.50-3.40 (m, 1H), 2.53 (s, | | Rt = 9.41 | | |
| | 3H), 2.22 (d, J = 13.4 Hz, 1H), | | min for | | |
| | 1.93-1.84 (m, 3H), 0.99-0.90 (m, 4H). | | Peak 2. | | |
| Ex. 98A | ¹H NMR (400 MHz, DMSO-d₆) δ | Condition | Chiral Pak | White | 23.2 |
| (Peak 1) | 9.65 (s, 1H), 8.95 (d, J = 8.1 Hz, | K: Rt = | IC (4.6 × | solid | |
| | 1H), 8.14 (d, J = 8.2 Hz, 1H), 7.72 | 2.76 min. | 250 mm), 5 μ | | |
| | (s, 1H), 7.38 (s, 1H), 4.51 (d, J = | m/z 512.4 | Mobile Phase: | | |
| | 11.1 Hz, 1H), 4.12-4.0 (m, 1H), | [M + H]⁺. | Hexane/DCM/ | | |
| | 3.74-3.63 (m, 2H), 3.58 (s, 3H), | | EtOH/IP | | |
| | 3.48-3.42 (m, 1H), 2.63 (s, 3H), | | Amine: | | |
| | 2.32-2.24 (m, 1H), 2.03-1.86 | | 20/40/40/0.1 | | |
| | (m, 3H), 1.01-0.87 (m, 4H). | | Flow Rate: | | |
| Ex. 98B | ¹H NMR (400 MHz, DMSO-d₆) δ | Condition | 1.0 ml/min | White | 21.5 |
| (Peak 2) | 9.65 (s, 1H), 8.95 (d, J = 7.9 Hz, | K: Rt = | Solubility: | solid | |
| | 1H), 8.14 (d, J = 8.2 Hz, 1H), 7.72 | 2.76 min. | MeOH Rt = | | |
| | (s, 1H), 7.38 (s, 1H), 4.51 (d, J = | m/z 512.4 | 5.05 min for | | |
| | 11.1 Hz, 1H), 4.10 (d, J = 10.9 Hz, | [M + H]⁺. | Peak 1 and | | |
| | 1H), 3.74-3.63 (m, 2H), 3.58 (s, | | Rt = 6.21 | | |
| | 3H), 3.50-3.40 (m, 1H), 2.63 (s, | | min for | | |
| | 3H), 2.32-2.24 (m, 1H), 2.0- | | Peak 2. | | |
| | 1.89 (m, 3H), 0.99-0.89 (m, 4H). | | | | |

### Example 99

### 2,3-dimethyl-6-(2-(1-methyl-1H-pyrazol-4-yl)morpholino)-8-(1-methyl-3-(trifluoromethyl)-1H-pyrazol-5-yl)pyrimido[5,4-d]pyrimidin-4 (3H)-one

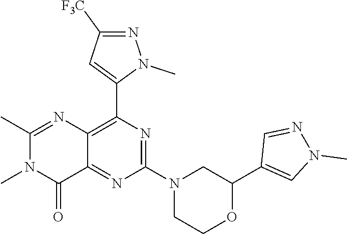

Example 99 was synthesized following similar protocol used to synthesize examples 4 to 10. Suzuki coupling was done following Condition-A.

¹H NMR (400 MHz, DMSO-d₆) δ 7.75 (s, 1H), 7.69 (s, 1H), 7.46 (s, 1H), 4.63 (d, J=13.1 Hz, 1H), 4.54-4.49 (m, 2H), 4.20 (s, 3H), 4.02 (d, J=10.1 Hz, 1H), 3.82 (s, 3H), 3.70-3.64 (m, 1H), 3.52 (s, 3H), 3.26-3.14 (s, 2H). 2.55 (s, 3H).

LCMS Condition K: Rt=2.72 min m/z 490.3 [M+H]⁺.

### Example 100

### Example-100A: Enantiomer 1

### (R)-6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one Example-100B: Enantiomer 2

### (S)-6-(2,2-dimethyl-6-(1-methyl-1H-pyrazol-4-yl)morpholino)-8-(2-fluoro-4-(trifluoro-5-methyl)phenyl)-2,3-dimethylpyrimido[5,4-d]pyrimidin-4 (3H)-one

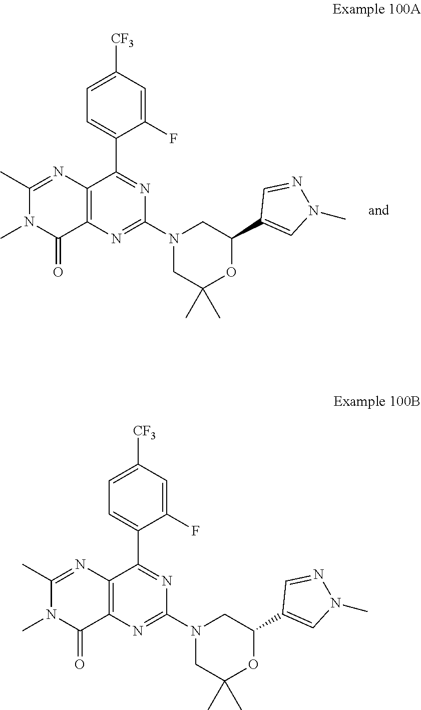

General procedure of Step-2, example-4 to 10 was followed using compounds [6-chloro-8-(2-fluoro-4-(trifluoro-15-methyl)phenyl)-2,3-dimethylpyrimido[5,4-d]pyrimidin-4 (3H)-one] and 2,2-dimethyl-6-(1-methyl-1H-pyrazol-4-yl)morpholine to afford 6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one as yellow solid in 60.7% yield. LCMS Condition H: Rt=2.96 min. m/z 532.41 [M+H]⁺.

Chiral separation of 6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one (1 g) was done by normal phase chiral prep HPLC to afford Example 100A, (R)-6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one (390 mg, 39%; eluted first, assigned as Peak 1 with arbitrary assignment of stereochemistry) as yellow solid and Example 100B, (S)-6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one (400 mg, 40% yield, eluted later, assigned as Peak 2 with arbitrary assignment of stereochemistry) as yellow solid.

Chiral prep HPLC method details to separate the enantiomers:

Instrument: Agilent 1200 series instrument/Column: CHIRALPAK IG (250×21 mm) 5u/Flow rate: 21.0 ml/min./Mobile phase: Hexane/Dichloromethane/Ethanol: 60/20/20/Solubility: Mixture of Acetonitrile and Dichloromethane/Wave length: 296 nm/Run time: 25 min

Example 100A (Peak 1): 1H NMR (400 MHz, DMSO-d6) δ 7.90-7.83 (m, 2H), 7.76-7.72 (m, 2H), 7.43 (s, 1H), 4.80-4.71 (m, 2H), 4.53 (d, J=12.5 Hz, 1H), 3.80 (s, 3H), 3.50 (s, 3H), 3.01-2.91 (m, 2H), 2.42 (s, 3H), 1.23 (s, 6H).

LCMS Condition H: Rt=2.98 min. m/z 532.4 [M+H]⁺.

Example 100B (Peak 2): 1H NMR (400 MHz, DMSO-d6) δ 7.88-7.83 (m, 2H), 7.76-7.72 (m, 2H), 7.43 (s, 1H), 4.80-4.71 (m, 2H), 4.53 (d, J=12.5 Hz, 1H), 3.80 (s, 3H), 3.50 (s, 3H), 3.01-2.91 (m, 2H), 2.42 (s, 3H), 1.24 (s, 6H).

LCMS Condition H: Rt=2.97 min. m/z 532.4 [M+H]+.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=4.97 min for Peak 1 and Rt=5.94 min for Peak 2.

### Example 101

### Example-101A: Enantiomer 1

### (R)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one

### Example-101B: Enantiomer 2

### (S)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one

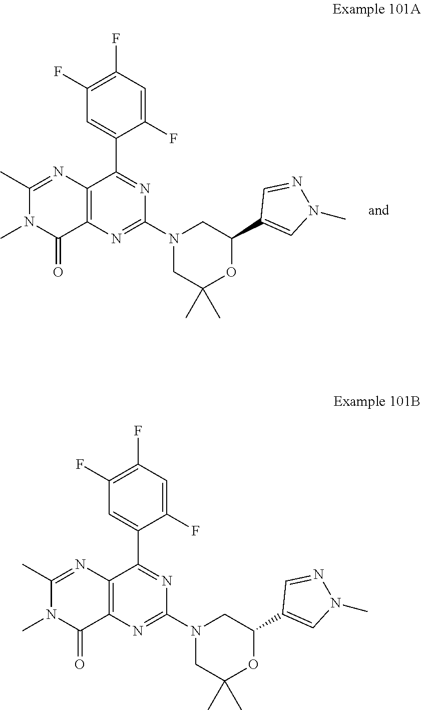

General procedure of Step-2, example-4 to 10 was followed using compounds [6-chloro-2,3-dimethyl-8-(2,4,5-trifluorophenyl)pyrimido[5,4-d]pyrimidin-4 (3H)-one and 2,2-dimethyl-6-(1-methyl-1H-pyrazol-4-yl)morpholine as starting materials to afford 6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one as yellow solid in 49.9% yield. Condition F: Rt=2.80 min. m/z 500.43 [M+H]⁺.

Chiral separation of 6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one (100 mg) was done by normal phase chiral prep HPLC to afford Example 101A, (R)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one (35 mg, 35%; eluted first, assigned as Peak 1 with arbitrary assignment of stereochemistry) as yellow solid and Example 100B, (S)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one (36 mg, 36% yield, eluted later, assigned as Peak 2 with arbitrary assignment of stereochemistry) as yellow solid. Chiral prep HPLC method details to separate the enantiomers:

Instrument: Agilent 1200 series instrument/Column: CHIRALPAK IG (250×21 mm) 5u/Flow rate: 21.0 ml/min./Mobile phase: Hexane/Dichloromethane/Ethanol: 60/20/20/Solubility: Mixture of Acetonitrile and Dichloromethane/Wave length: 298 nm/Run time: 36 min

Example 101A (Peak 1): 1H NMR (400 MHz, DMSO-d6) δ 7.81-7.68 (m, 3H), 7.43 (s, 1H), 4.79-4.71 (m, 2H), 4.53 (d, J=12.7 Hz, 1H), 3.81 (s, 3H), 3.50 (s, 3H), 3.0-2.90 (m, 2H), 2.44 (s, 3H) 1.24 (d, J=4.6 Hz, 6H).

LCMS Condition H: Rt=2.82 min. m/z 500.4 [M+H]⁺.

Absolute stereochemistry unknown

Example 101B (Peak 2): 1H NMR (400 MHz, DMSO-d6) δ 7.79-7.68 (m, 3H), 7.43 (s, 1H), 4.79-4.71 (m, 2H), 4.53 (d, J=12.4 Hz, 1H), 3.81 (s, 3H), 3.50 (s, 3H), 3.0-2.90 (m, 2H), 2.44 (s, 3H) 1.24-1.23 (m, 6H).

LCMS Condition H: Rt=2.82 min. m/z 500.4 [M+H]⁺.

Absolute stereochemistry unknown

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=5.60 min for Peak 1 and Rt=10.45 min for Peak 2.

### Example 102

### Example-102A: Enantiomer 1

### (R)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one

### Example-102B: Enantiomer 2

### (S)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one

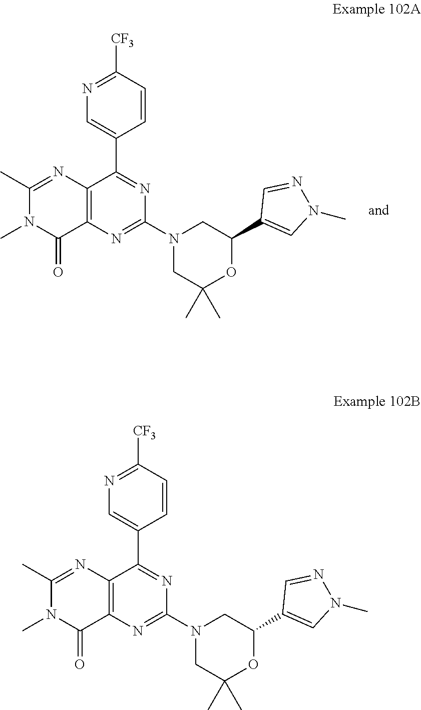

General procedure of Step-2, example-4 to 10 was followed using compounds 6-chloro-2,3-dimethyl-8-(6-(trifluoromethyl)pyridin-3-yl)pyrimido[5,4-d]pyrimidin-4 (3H)-one and 2,2-dimethyl-6-(1-methyl-1H-pyrazol-4-yl)morpholine as starting materials to afford 6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one as yellow solid in 55.2% yield. Condition P: Rt=2.81 min. m/z 515.41 [M+H]⁺.

Chiral separation of 6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one (160 mg) was done by normal phase chiral prep HPLC to afford Example 102A, (R)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one (38 mg, 23.7%; eluted first, assigned as Peak 1 with arbitrary assignment of stereochemistry) as yellow solid and Example 102B, (S)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one (37 mg, 23.1% yield, eluted later, assigned as Peak 2 with arbitrary assignment of stereochemistry) as yellow solid.

Chiral prep HPLC method details to separate the enantiomers:

Instrument: Agilent 1200 series instrument/Column: CHIRALPAK IG (250×21 mm) 5u/Flow rate: 21.0 ml/min./Mobile phase: Hexane/Dichloromethane/Ethanol: 70/15/15/Solubility: Mixture of Acetonitrile and Dichloromethane/Wave length: 302 nm/Run time: 20 min

Example 102A (Peak 1): 1H NMR (400 MHz, DMSO-d6) δ 9.58 (s, 1H), 8.91 (d, J=8.0 Hz, 1H), 8.10 (d, J=8.3 Hz, 1H), 7.74 (s, 1H), 7.46 (s, 1H), 4.81 (d, J=9.7 Hz, 2H), 4.63 (d, J=12.2 Hz, 1H), 3.82 (s, 3H), 3.53 (s, 3H), 3.0-2.94 (m, 2H), 2.54 (s, 3H), 1.27-1.25 (m, 6H).

LCMS Condition H: Rt=2.83 min. m/z 515.4 [M+H]⁺.

Absolute stereochemistry unknown

Example 102B (Peak 2): 1H NMR (400 MHz, DMSO-d6) δ 9.58 (s, 1H), 8.90 (d, J=7.9 Hz, 1H), 8.10 (d, J=8.3 Hz, 1H), 7.74 (s, 1H), 7.46 (s, 1H), 4.81 (d, J=9.5 Hz, 2H), 4.63 (d, J=12.7 Hz, 1H), 3.82 (s, 3H), 3.53 (s, 3H), 3.03-2.94 (m, 2H), 2.54 (s, 3H), 1.27-1.25 (m, 6H).

LCMS Condition H: Rt=2.83 min. m/z 515.4 [M+H]+.

Absolute stereochemistry unknown

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=8.62 min for Peak 1 and Rt=9.35 min for Peak 2.

### Example 103

### (S)-8-(4-chloro-2-fluorophenyl)-3-methyl-6-(2-(1-methyl-1H-pyrazol-4-yl)morpholino)-2-(trifluoromethyl)pyrimido[5,4-d]pyrimidin-4 (3H)-one

Compounds 103 was prepared using similar procedures as described in Example 1.

| Ex. #/Structure | Name | LCMS/NMR | Description | Yield (%) |
|---|---|---|---|---|
| Step-1 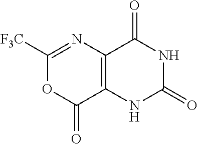 [SMILES: O=c1[nH]c(=O)c2nc(C(F)(F)F)oc(=O)c2[nH]1] | 2- (trifluoromethyl)- 4H-pyrimido[5,4- d][1,3]oxazine- 4,6,8(5H,7H)- trione | ¹H-NMR (400 MHz, DMSO-d6): δ 11.81 (s, 1H), 11.64 (s, 1H) | brown solid | 68.0 |
| Step-2 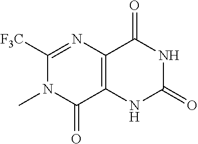 [SMILES: Cn1c(C(F)(F)F)nc2c(=O)[nH]c(=O)[nH]c2c1=O] | 7-methyl-6- (trifluoromethyl)- 1,7- dihydropyrimido [5,4-d]pyrimidine- 2,4,8(3H)-trione | LCMS Condition A: Rt = 1.14 min. m/z 262.1 [M + H]+ | brown solid | 12.0 |
| Step-3 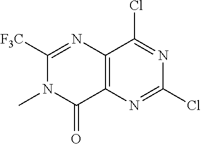 [SMILES: Cn1c(C(F)(F)F)nc2c(Cl)nc(Cl)nc2c1=O] | 6,8-dichloro-3- methyl-2- (trifluoromethyl) pyrimido[5,4-d] pyrimidin-4(3H)- one | LCMS Condition P: Rt = 3.26 min. m/z 299.0 [M + H]+ | brown solid | 20.0 |
| Step-4 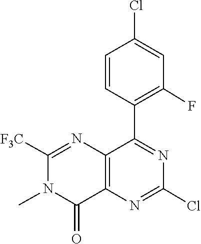 [SMILES: Cn1c(C(F)(F)F)nc2c(-c3ccc(Cl)cc3F)nc(Cl)nc2c1=O] | 6-chloro-8-(4- chloro-2- fluorophenyl)-3- methyl-2- (trifluoromethyl) pyrimido[5,4- d]pyrimidin- 4(3H)-one | LCMS Condition H: Rt = 3.62 min. m/z 393.0 [M + H]+ Note: Suzuki performed at rt | Off white solid | 50.5 |
| Ex. 103 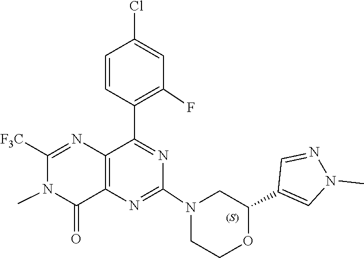 | (S)-8-(4-chloro-2- fluorophenyl)-3- methyl-6-(2-(1- methyl-1H- pyrazol-4- yl)morpholino)-2- (trifluoromethyl) pyrimido[5,4- d]pyrimidin- 4(3H)-one | LCMS Condition H: Rt = 2.97 min. m/z 524.1 [M + H]⁺. Note: [Use chiral Preparation-1, Peak-1] | Yellow solid | 22.0 |

Ex. 103 ¹H NMR (400 MHz, DMSO-d₆) δ 7.75-7.64 (m, 3H), 7.49-7.46 (m, 2H), 4.70-4.50 (m, 3H), 4.10-4.0 (m, 1H), 3.81 (s, 3H), 3.66 (t, J=10.6 Hz, 1H), 3.55 (s, 3H), 3.29-3.22 (m, 2H).

### Example 104

### 8-(4-chloro-2-fluorophenyl)-2,3-dimethyl-6-(2-(1-methyl-1H-pyrazol-4-yl)morpholino)pyrido[3,2-d]pyrimidin-4 (3H)-one

### Step-1—Preparation of methyl 3-amino-4-bromo-6-chloropicolinate

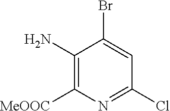 [SMILES: COC(=O)c1nc(Cl)cc(Br)c1N]

To a stirred solution of methyl 3-amino-6-chloropicolinate (1.0 g, 5.38 mmol) in DMF (5.0 mL) was added NBS (1.44 g, 8.07 mmol) at 10° C. Resulting mixture was heated at 80° C. for 2 h. Reaction mixture was quenched with crushed ice, precipitate thus formed was filtered, washed with ice cold water and dried to afford methyl 3-bromo-5-(2-(1-methyl-1H-pyrazol-4-yl)morpholino)-2-nitrobenzoate (1.2 g, yield 84.1%) as off white solid.

LCMS Condition E: Rt=1.92 min. m/z 265.1 [M+H]⁺.

### Step-2—Preparation of methyl 3-acetamido-4-bromo-6-chloropicolinate

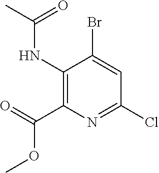 [SMILES: COC(=O)c1nc(Cl)cc(Br)c1NC(C)=O]

To a stirred solution of methyl 3-bromo-5-(2-(1-methyl-1H-pyrazol-4-yl)morpholino)-2-nitrobenzoate (1.2 g, 4.55 mmol) in DMF (10.0 mL) was added K2CO3 (1.25 g, 9.09 mmol) portion wise. Resulting mixture was heated at 80° C. for 2 h. After 2 h, reaction mixture was cooled to 0° C. and acetyl chloride (0.65 mL, 13.64 mmol) was added dropwise. Resulting mixture was then stirred at rt for 16 h. Reaction mixture was quenched with ice cold water and extracted with ethyl acetate. Combined organic part was washed with water, brine, dried over anhydrous sodium sulphate, filtered and concentrated under reduced pressure. Crude mass was purified by column chromatography (30-50% ethyl acetate-hexane) to afford methyl 3-acetamido-4-bromo-6-chloropicolinate (800 mg, 57.2% yield) as off white solid.

LCMS Condition F: Rt=1.67 min. m/z 307.2 [M+H]⁺.

### Step-3—Preparation of 3-acetamido-4-bromo-6-chloropicolinic acid

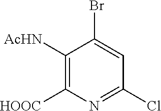 [SMILES: CC(=O)Nc1c(Br)cc(Cl)nc1C(=O)O]

To a stirred solution of methyl 3-acetamido-4-bromo-6-chloropicolinate (800 mg, 2.62 mmol) in THF (10 mL) and H2O (3 mL) was added lithium hydroxide (275.15 mg, 6.56 mmol) portion wise at 5° C. The resulting mixture was stirred at RT for 1 h. Reaction mixture was concentrated under reduced pressure, diluted with water and washed with diethyl ether. Aqueous portion was cooled to 10° C., acidified with 1(N) solution and extracted with 10% MeOH-DCM. Organic part was dried over anhydrous sodium sulphate, filtered and concentrated under reduced pressure to afford 3-acetamido-4-bromo-6-chloropicolinic acid (650 mg. 84.4% yield) as yellow solid.

LCMS Condition J: Rt=0.88 min. m/z 293.2 [M+H]⁺.

### Step-4—Preparation of 8-bromo-6-chloro-2,3-dimethylpyrido[3,2-d]pyrimidin-4 (3H)-one

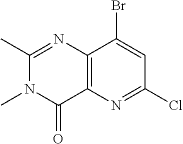

To a stirred solution of 3-acetamido-4-bromo-6-chloropicolinic acid (400 mg, 1.36 mmol) in Pyridine (5.0 mL) was added MeNH2 in THF (2M, 4.0 ml) at 5° C. Reaction mixture was stirred at RT for 15 mins. It was again cooled to 0° C. and T3P (50% in EA solution, 2.0 ml, 2.73 mmol) was added dropwise. Resulting mixture was stirred at RT for 16 h. Reaction mixture was concentrated, quenched with water, extracted with ethyl acetate. Organic part was washed with water, brine, dried over anhydrous sodium sulphate, filtered and concentrated under reduced pressure. Crude mass was purified by column chromatography (30-50% ethyl acetate-hexane) to afford 8-bromo-6-chloro-2,3-dimethylpyrido[3,2-d]pyrimidin-4 (3H)-one (100 mg, 25.4% yield) as white solid.

LCMS Condition G: Rt=2.58 min. m/z 288.2 [M+H]⁺.

### Step-5—Preparation of 6-chloro-8-(4-chloro-2-fluorophenyl)-2,3-dimethylpyrido[3,2-d]pyrimidin-4 (3H)-one

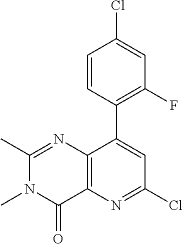 [SMILES: Cc1nc2c(-c3ccc(Cl)cc3F)cc(Cl)nc2c(=O)n1C]

To a stirred solution of 8-bromo-6-chloro-2,3-dimethylpyrido[3,2-d]pyrimidin-4 (3H)-one (100.0 mg, 0.35 mmol) and (4-chloro-2-fluorophenyl)boronic acid (54.47 gm, 0.31 mmol) in dioxane (6 mL) and water (2 mL) was added sodium carbonate (73.54 mg, 0.69 mmol) and degassed with argon. PdCl2(dppf) (25.38 mg, 0.035 mmol) was added under inert atmosphere. The resulting mixture was heated at 80° C. for 1 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (35% ethyl acetate-hexane) to afford 6-chloro-8-(4-chloro-2-fluorophenyl)-2,3-dimethylpyrido[3,2-d]pyrimidin-4 (3H)-one (60 mg, 51.1% yield) as off white solid.

LCMS Condition G: Rt=2.14 min. m/z 338.3 [M+H]⁺.

### Step-6—Preparation of 8-(4-chloro-2-fluorophenyl)-2,3-dimethyl-6-(2-(1-methyl-1H-pyrazol-4-yl)morpholino)pyrido[3,2-d]pyrimidin-4 (3H)-one

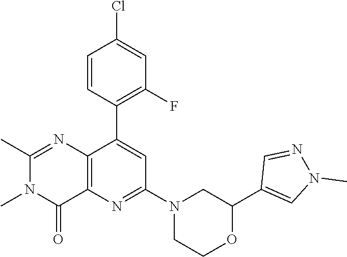

Procedure of Step-5, example-1 was followed using 6-chloro-8-(4-chloro-2-fluorophenyl)-2,3-dimethylpyrido[3,2-d]pyrimidin-4 (3H)-one and 2-(1-methyl-1H-pyrazol-4-yl)morpholine (HCl salt) as starting materials to afford 8-(4-chloro-2-fluorophenyl)-2,3-dimethyl-6-(2-(1-methyl-1H-pyrazol-4-yl)morpholino)pyrido[3,2-d]pyrimidin-4 (3H)-one (15.97% yield) as yellow solid.

1H NMR (400 MHz, DMSO D6) δ 7.61-5.80 (m, 2H), 7.47-7.44 (m, 2H), 7.19 (s, 1H), 4.24 (t, J=8.2 Hz, 2H), 4.06 (d, J=6.4 Hz, 2H), 3.96-3.92 (m, 2H), 3.72 (s, 3H), 3.49 (s, 3H), 3.13-3.10 (m, 1H), 2.42 (s, 3H).

LCMS Condition K: Rt=2.70 min. m/z 469.4 [M+H]⁺.

### Example 105

### 8-(4-chloro-2-fluorophenyl)-2,3-dimethyl-6-(2-(1-methyl-1H-pyrazol-4-yl)morpholino)pyrido[3,2-d]pyrimidin-4 (3H)-one

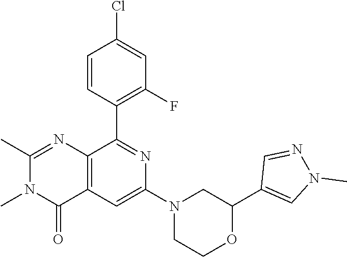

Example 105 was synthesized following protocol described to synthesize Example 104 using methyl 5-amino-2-chloroisonicotinate [commercial] in 6 steps.

1H NMR (400 MHz, DMSO-d6) δ 7.75 (s, 1H), 7.61-7.49 (m, 2H), 7.46 (s, 1H), 7.40 (dd, J=2.1, 8.2 Hz, 1H), 7.31 (s, 1H), 4.55 (d, J=8.0 Hz, 1H), 4.29 (d, J=12.7 Hz, 1H), 4.12 (d, J=12.8 Hz, 1H), 4.02 (d, J=11.1 Hz, 1H), 3.81 (s, 3H), 3.70 (t, J=9.4 Hz, 1H), 3.51 (s, 3H), 3.07-2.88 (m, 2H), 2.43 (s, 3H).

LCMS Condition K: Rt=2.79 min. m/z 469.3 [M+H]⁺.

### Example 106

### 8-(4-chloro-2-fluorophenyl)-2,3-dimethyl-6-(2-(1-methyl-1H-pyrazol-4-yl)morpholino)quinazolin-4 (3H)-one

### Step-1—Preparation of methyl 3-bromo-5-(2-(1-methyl-1H-pyrazol-4-yl)morpholino)-2-nitrobenzoate

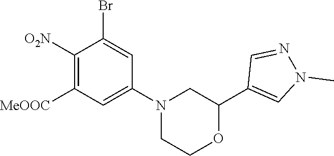 [SMILES: COC(=O)c1cc(N2CCOC(c3cnn(C)c3)C2)cc(Br)c1[N+](=O)[O-]]

Procedure of Step-5, example-1 was followed using methyl 3-bromo-5-fluoro-2-nitrobenzoate and 2-(1-methyl-1H-pyrazol-4-yl)morpholine (HCl salt) as starting materials to afford 3-bromo-5-(2-(1-methyl-1H-pyrazol-4-yl)morpholino)-2-nitrobenzoate (54.4% yield) as brown gum.

LCMS Condition C: Rt=2.02 min. m/z 426.2 [M+H]⁺.

### Step-2—Preparation of methyl 4′-chloro-2′-fluoro-5-(2-(1-methyl-1H-pyrazol-4-yl)morpholino)-2-nitro-[1,1′-biphenyl]-3-carboxylate

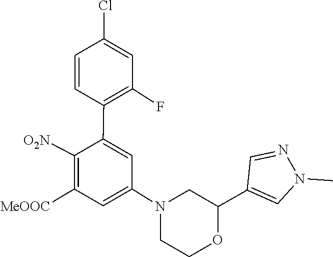 [SMILES: COC(=O)c1cc(N2CCOC(c3cnn(C)c3)C2)cc(-c2ccc(Cl)cc2F)c1[N+](=O)[O-]]

Procedure of Step-5, example-104 was followed using 3-bromo-5-(2-(1-methyl-1H-pyrazol-4-yl)morpholino)-2-nitrobenzoate and (4-chloro-2-fluorophenyl)boronic acid as starting materials to afford methyl 4′-chloro-2′-fluoro-5-(2-(1-methyl-1H-pyrazol-4-yl)morpholino)-2-nitro-[1,1′-biphenyl]-3-carboxylate (71.8% yield) as off-white solid.

LCMS Condition J: Rt=2.20 min. m/z 475.2 [M+H]⁺.

### Step-3—Preparation of methyl 2-amino-4′-chloro-2′-fluoro-5-(2-(1-methyl-1H-pyrazol-4-yl)morpholino)-[1,1′-biphenyl]-3-carboxylate

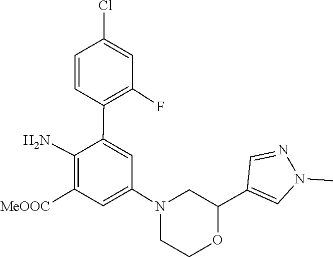 [SMILES: COC(=O)c1cc(N2CCOC(c3cnn(C)c3)C2)cc(-c2ccc(Cl)cc2F)c1N]

To a stirred solution of methyl 4′-chloro-2′-fluoro-5-(2-(1-methyl-1H-pyrazol-4-yl)morpholino)-2-nitro-[1,1′-biphenyl]-3-carboxylate (160 mg, 0.34 mmol) in Ethanol (5 mL) was added SnCl2·2H2O (759.49 mg, 3.38 mmol). Resulting mixture was heated at 90° C. for 16 h. The reaction mass was diluted with water and extracted with ethyl acetate Combined organic part was washed with water, brine, dried over anhydrous sodium sulphate, filtered and concentrated under reduced pressure. Crude mass was purified by column chromatography (0-3% MeOH-DCM) to afford methyl 2-amino-4′-chloro-2′-fluoro-5-(2-(1-methyl-1H-pyrazol-4-yl)morpholino)-[1,1′-biphenyl]-3 carboxylate (130 mg, 86.6% yield) as sticky solid.

LCMS Condition J: Rt=2.04 min. m/z 445.3 [M+H]⁺.

### Step-4—Preparation of 2-amino-4′-chloro-2′-fluoro-5-(2-(1-methyl-1H-pyrazol-4-yl)morpholino)-[1,1′-biphenyl]-3-carboxylic acid

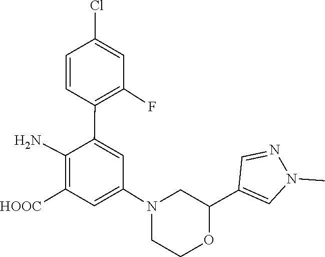 [SMILES: Cn1cc(C2CN(c3cc(C(=O)O)c(N)c(-c4ccc(Cl)cc4F)c3)CCO2)cn1]

To a stirred solution of methyl 2-amino-4′-chloro-2′-fluoro-5-(2-(1-methyl-1H-pyrazol-4-yl)morpholino)-[1,1′-biphenyl]-3 carboxylate (130 mg, 0.29 mmol) in Methanol (2 ml), THF (2 ml) and water (1 ml) was added sodium hydroxide (29.28 mg, 0.73 mmol) at 0° C. Resulting mixture was heated at 60° C. for 4 h. The reaction mass was concentrated under vacuum. Crude reaction mass was acidified by 1(N) HCl and extracted with ethyl acetate. Combined organic part was dried over sodium sulphate, filtered and concentrated under reduced pressure to afford 2-amino-4′-chloro-2′-fluoro-5-(2-(1-methyl-1H-pyrazol-4-yl)morpholino)-[1,1′-biphenyl]-3-carboxylic acid (100 mg, 79.3% yield) as off white solid.

LCMS Condition F: Rt=1.97 min. m/z 431.4 [M+H]⁺.

### Step-5—Preparation of 6-chloro-8-(4-chloro-2-fluorophenyl)-2,3-dimethylpyrido[3,2-d]pyrimidin-4 (3H)-one

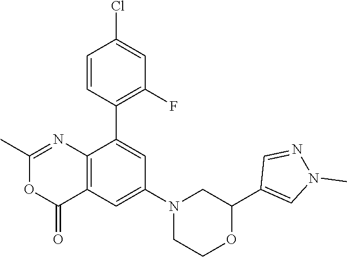

To a stirred solution of -amino-4′-chloro-2′-fluoro-5-(2-(1-methyl-1H-pyrazol-4-yl)morpholino)-[1,1′-biphenyl]-3-carboxylic acid (100 mg, 0.303 mmol) in Pyridine (0.5 mL) was added acetic anhydride (0.17 mL, 1.82 mmol) at 5° C. Resulting mixture was stirred at RT for 2 h. Reaction mixture was concentrated under reduced pressure and triturated with diethyl ether and dried to afford 6-chloro-8-(4-chloro-2-fluorophenyl)-2,3-dimethylpyrido[3,2-d]pyrimidin-4 (3H)-one (100 mg, 72.5% yield)

LCMS Condition J: Rt=1.96 min. m/z 455.3 [M+H]⁺.

### Step-6—Preparation of 8-(4-chloro-2-fluorophenyl)-2,3-dimethyl-6-(2-(1-methyl-1H-pyrazol-4-yl)morpholino)quinazolin-4 (3H)-one

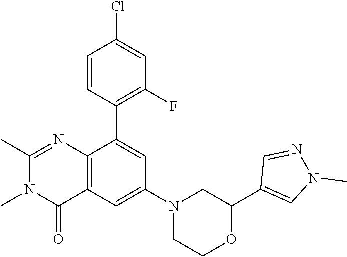

To a stirred solution of 6-chloro-8-(4-chloro-2-fluorophenyl)-2,3-dimethylpyrido[3,2-d]pyrimidin-4 (3H)-one (80 mg, 0.18 mmol) in THF (2 mL) was added 2(M) MeNH2 in THF (0.18 mL, 0.352 mmol) at 0° C. Resulting mixture was heated at 120° C. for 48 h. Reaction mixture was concentrated under reduced pressure and was purified by reverse phase prep HPLC method to afford 8-(4-chloro-2-fluorophenyl)-2,3-dimethyl-6-(2-(1-methyl-1H-pyrazol-4-yl)morpholino)quinazolin-4 (3H)-one (20 mg, 24.3% yield) as white solid.

1H NMR (400 MHz, DMSO-d6) δ 7.73 (s, 1H), 7.56 (d, J=2.3 Hz, 1H), 7.55-7.41 (m, 4H), 7.36 (dd, J=2.1, 8.2 Hz, 1H), 4.65-4.56 (m, 1H), 4.03 (d, J=10.6 Hz, 1H), 3.87 (d, J=11.6 Hz, 1H), 3.80 (s, 3H), 3.80-3.72 (m, 1H), 3.67 (d, J=12.0 Hz, 1H), 3.51 (s, 3H), 2.93-2.72 (m, 2H), 2.41 (s, 3H).

LCMS Condition K: Rt=2.82 min. m/z 468.3 [M+H]⁺.

### Example 107

### Example-107A: Enantiomer 1

### (R)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one

### Example-107B: Enantiomer 2

### (S)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one

### Step-1—Preparation of 6-(2-(1-(bromodifluoromethyl)-1H-pyrazol-4-yl)morpholino)-8-(4-chloro-2-fluorophenyl)-2,3-dimethylpyrimido[5,4-d]pyrimidin-4 (3H)-one

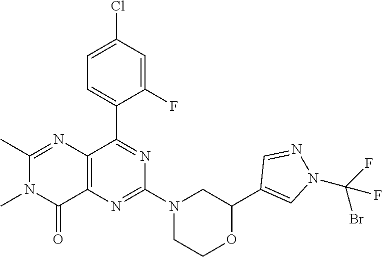 [SMILES: Cc1nc2c(-c3ccc(Cl)cc3F)nc(N3CCOC(c4cnn(C(F)(F)Br)c4)C3)nc2c(=O)n1C]

To a solution of 6-(2-(1H-pyrazol-4-yl)morpholino)-8-(4-chloro-2-fluorophenyl)-2,3dimethylpyrimido[5,4-d]pyrimidin-4 (3H)-one (350.0 mg, 0.77 mmol, Example 4-10) in DMF (5 mL) was added cesium carbonate (500.0 mg, 1.54 mmol) and dibromo difluoromethane (0.37 mL, 15.4 mmol). The reaction mixture was stirred at room temperature overnight, diluted with 20 mL DCM and washed with water. The organic phase was separated and concentrated under vacuo and the crude product was purified by column chromatography eluting with a gradient of (0-50)% EtOAc in hexane to afford 6-(2-(1-(bromodifluoromethyl)-1H-pyrazol-4-yl)morpholino)-8-(4-chloro-2-fluorophenyl)-2,3-dimethylpyrimido[5,4-d]pyrimidin-4 (3H)-one (200.0 mg, 44.5% yield) as yellow solid.

1H NMR (400 MHz, DMSO D6) δ 8.50 (s, 1H), 8.05 (s, 1H), 7.65 (t, J=8.04 Hz, 1H), 7.60 (d, J=9.72 Hz, 1H), 7.45 (d, J=8.24 Hz, 1H), 4.68 (d, J=12.32 Hz, 1H), 4.62 (d, J=10.32 Hz, 1H), 4.53 (d, J=13.64 Hz, 1H), 4.05-4.02 (m, 1H), 3.69 (t, J=11.96 Hz, 1H), 3.51 (s, 3H), 3.25-3.18 (m, 2H), 2.44 (s, 3H).

LCMS Condition B: Rt=1.96 min. m/z 584.0 [M+H]⁺.

### Step-2—Preparation of 8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one

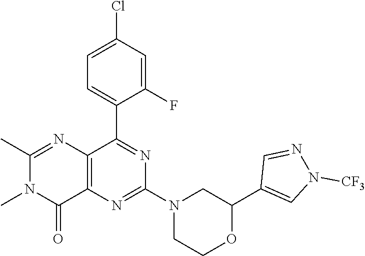 [SMILES: Cc1nc2c(-c3ccc(Cl)cc3F)nc(N3CCOC(c4cnn(C(F)(F)F)c4)C3)nc2c(=O)n1C]

To a stirred solution of 6-(2-(1-(bromodifluoromethyl)-1H-pyrazol-4-yl)morpholino)-8-(4-chloro-2-fluorophenyl)-2,3-dimethylpyrimido[5,4-d]pyrimidin-4 (3H)-one (200.0 mg, 0.343 mmol) in DCM (8 mL) was added silver(I) tetrafluoroborate (400.35 mg, 2.06 mmol) at −78° C. Then the solution was stirred at room temperature overnight. Progress of the reaction was monitored by LC-Ms analysis that confirmed formation of product mass. After completion, the mixture was diluted with water and followed by extracted with DCM containing 5% MeOH. Combined organic layer was dried over magnesium sulfate and concentrated into vacuo. The crude product was purified by column chromatography eluting with a gradient of (0-50)% EtOAc in hexane to afford 8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one (100.0 mg, 55.7% yield) as yellow solid.

1H NMR (400 MHz, DMSO D6) δ 8.50 (s, 1H), 8.05 (s, 1H), 7.67 (t, J=8.04 Hz, 1H), 7.60 (d, J=9.72 Hz, 1H), 7.45 (d, J=8.96 Hz, 1H), 4.69 (d, J=12.72 Hz, 1H), 4.63 (d, J=10.36 Hz, 1H), 4.54 (d, J=13.08 Hz, 1H), 4.06-4.0 (m, 2H), 3.70 (t, J=10.48 Hz, 1H), 3.51 (s, 3H), 3.25-3.18 (m, 2H), 2.44 (s, 3H).

LCMS Condition B: Rt=3.046 min. m/z 524.08 [M+H]⁺.

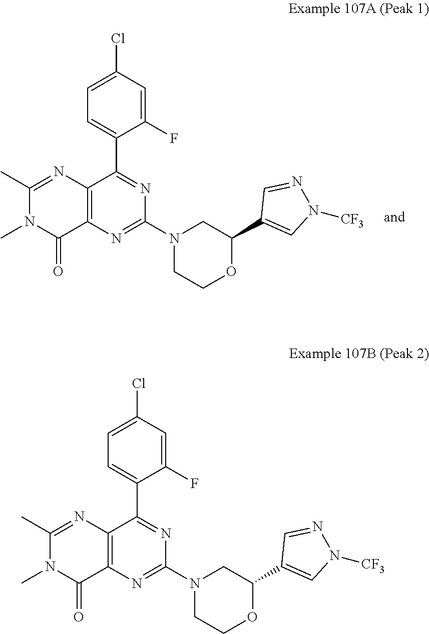 [SMILES: Cc1nc2c(-c3ccc(Cl)cc3F)nc(N3CCO[C@H](c4cnn(C(F)(F)F)c4)C3)nc2c(=O)n1C ; Cc1nc2c(-c3ccc(Cl)cc3F)nc(N3CCO[C@@H](c4cnn(C(F)(F)F)c4)C3)nc2c(=O)n1C]

Chiral separation of 8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one (100 mg) was done by normal phase chiral prep HPLC to afford Example 107A, (R)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one (35.4 mg, 35.4%; eluted first, assigned as Peak 1 with arbitrary assignment of stereochemistry) as yellow solid and Example 107B, (S)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one (32.4 mg, 32.4% yield, eluted later, assigned as Peak 2 with arbitrary assignment of stereochemistry) as yellow solid.

Chiral prep HPLC method details to separate the enantiomers:

Instrument: Agilent 1200 series instrument/Column: CHIRALPAK IC (250×21 mm) 5u/Flow rate: 21.0 ml/min./Mobile phase: Hexane/Dichloromethane/Ethanol: 50/25/25/Solubility: Mixture of Acetonitrile and Dichloromethane/Wave length: 295 nm/Run time: 36 min

Example 107A (Peak 1): ¹H NMR (400 MHz, DMSO-d₆) δ 8.58 (s, 1H), 8.05 (s, 1H), 7.68-7.60 (m, 2H), 7.47-7.44 (m, 1H), 4.70-4.61 (m, 2H), 4.55 (d, J=12.9 Hz, 1H), 4.05 (d, J=10.2 Hz, 1H), 3.72-3.67 (m, 1H), 3.51 (s, 3H), 3.24-3.18 (m, 2H), 2.44 (s, 3H).

LCMS Condition P: Rt=3.07 min. m/z 524.3 [M+H]⁺.

Absolute stereochemistry unknown Example 107B (Peak 2): ¹H NMR (400 MHz, DMSO-d₆) δ 8.58 (s, 1H), 8.05 (s, 1H), 7.66 (t, J=8.0 Hz, 1H), 7.61 (dd, J=1.7 Hz, 9.7 Hz, 1H), 7.46 (dd, J=1.7, 8.1 Hz, 1H), 4.67-4.53 (m, 3H), 4.10-4.0 (m, 1H), 3.70-3.60 (m, 1H), 3.51 (s, 3H), 3.21 (t, J=11.7 Hz, 2H), 2.50-2.44 (m, 3H).

LCMS Condition P: 3.07 min. m/z 524.3 [M+H]⁺.

Absolute stereochemistry unknown

Analytical chiral HPLS: Chiralpak IG (4.6×250 mm), 5μ Mobile Phase: Hexane/EtOH/DCM/IPAmine: 60/20/20/0.1 Flow Rate: 1.0 ml/min. Solubility: MeOH, Rt=5.27 min for Peak 1 and Rt=5.86 min for Peak 2.

### Example 108

### 8-(4-chloro-2-fluorophenyl)-2,3-dimethyl-6-(5-(1-methyl-1H-pyrazol-4-yl)-4-oxa-7-azaspiro(2,5)octan-7-yl)pyrimido(5,4-d)pyrimidin-4 (3H)-one

Compound 108 was prepared following the procedure as described in Preparation-1 steps 2-5, and Step-6 was followed as described in Example 43-58.

| | | | | | |
|---|---|---|---|---|---|
| Ex. # | Structure | Name | LCMS/NMR | Description | Yield (%) |

| | | | | |
|---|---|---|---|---|
| Step-2 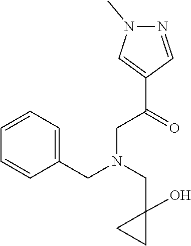 [SMILES: Cn1cc(C(=O)CN(Cc2ccccc2)CC2(O)CC2)cn1] | 2-(benzyl(1- hydroxycyclopropyl) methyl)amino-1-(1- methyl-1-H-pyrazol-4- yl)ethan-1-one | LCMS Condition A: Rt = 3.34 min. m/z 300.0 [M + H]⁺ | Gummy | 77.0 |
| Step-3 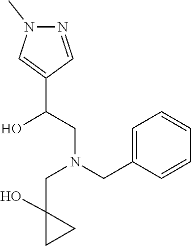 [SMILES: Cn1cc(C(O)CN(Cc2ccccc2)CC2(O)CC2)cn1] | 1-((benzyl(2-hydroxy- 2-(1-methyl-1-H- pyrazol-4- yl)ethyl)amino)methyl) cyclopropan-1-ol | LCMS Condition A: Rt = 2.85 min. m/z 302.2 [M + H]⁺. | Gummy | 85.8 |
| Step-4 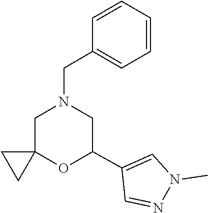 [SMILES: Cn1cc(C2CN(Cc3ccccc3)CC3(CC3)O2)cn1] | 7-benzyl-5-(1-methyl- 1H-pyrazol-4-yl)-5- oxa-7-azaspiro(2,5)- octane | LCMS Condition A: Rt = 3.13 min. m/z 283.9 [M + H]⁺. | Gummy | 79.7 |
| Step-5 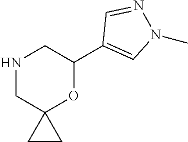 | 5-(1-methyl-1H- pyrazol-4-yl)-4-oxa-7- azaspiro(2,5)-octane | LCMS Condition A: Rt = 0.94 min. m/z 194.1 [M + H]⁺. | Gummy | 87.8 |
| Ex. 108 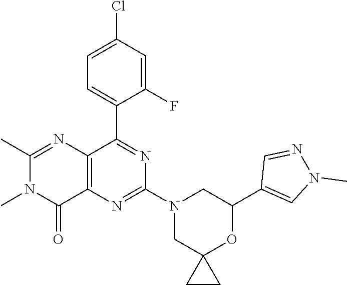 | 8-(4-chloro-2- fluorophenyl)-2,3- dimethyl-6-(5-(1- methyl-1H-pyrazol-4- yl)-4-oxa-7- azaspiro(2,5)octan-7- yl)pyrimido(5,4- d)pyrimidin-4(3H)-one | LCMS Condition P: Rt = 2.87 min. m/z 496.4 [M + H]⁺. | Yellow solid | 13.0 |

Ex. 108 ¹H NMR (400 MHz, DMSO-d₆) δ 7.74 (s, 1H), 7.67-7.59 (m, 2H), 7.44 (s, 2H), 4.80-4.63 (m, 2H), 4.08 (d, J=13.5 Hz, 1H), 3.80 (s, 3H), 3.63 (d, J=13.7 Hz, 1H), 3.50 (s, 3H), 3.22-3.16 (m, 1H), 2.49-2.43 (m, 3H), 0.90-0.50 (m, 4H).

### Example 108A and Example 108B

### (R)-8-(4-chloro-2-fluorophenyl)-2,3-dimethyl-6-(5-(1-methyl-1H-pyrazol-4-yl)-4-oxa-7-azaspiro[2.5]octan-7-yl)pyrimido[5,4-d]pyrimidin-4 (3H)-one and (S)-8-(4-chloro-2-fluorophenyl)-2,3-dimethyl-6-(5-(1-methyl-1H-pyrazol-4-yl)-4-oxa-7-azaspiro[2.5]octan-7-yl)pyrimido[5,4-d]pyrimidin-4 (3H)-one

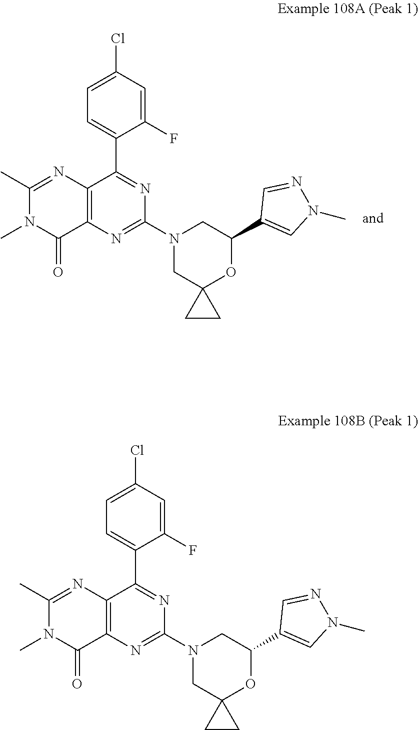

Chiral separation of 8-(4-chloro-2-fluorophenyl)-2,3-dimethyl-6-(5-(1-methyl-1H-pyrazol-4-yl)-4-oxa-7-azaspiro(2,5)octan-7-yl)pyrimido(5,4-d)pyrimidin-4 (3H)-one (Example 108, 130 mg) was done by normal phase chiral prep HPLC to afford Example 108A, (R)-8-(4-chloro-2-fluorophenyl)-2,3-dimethyl-6-(5-(1-methyl-1H-pyrazol-4-yl)-4-oxa-7-azaspiro[2.5]octan-7-yl)pyrimido[5,4-d]pyrimidin-4 (3H)-one (43.5 mg, 33.4%; eluted first, assigned as Peak 1 with arbitrary assignment of stereochemistry) as yellow solid and Example 108B, (S)-8-(4-chloro-2-fluorophenyl)-2,3-dimethyl-6-(5-(1-methyl-1H-pyrazol-4-yl)-4-oxa-7-azaspiro[2.5]octan-7-yl)pyrimido[5,4-d]pyrimidin-4 (3H)-one (42.24 mg, 32.5% yield, eluted later, assigned as Peak 2 with arbitrary assignment of stereochemistry) as yellow solid.

Chiral prep HPLC method details to separate the enantiomers:

Instrument: Agilent 1200 series instrument/Column: CHIRALPAK IG (250×21 mm) 5u/Flow rate: 21.0 ml/min./Mobile phase: Hexane/Dichloromethane/Ethanol: 60/20/20/Solubility: Mixture of Acetonitrile and Dichloromethane/Wave length: 296 nm/Run time: 20 min

Example 108A (Peak 1): 1H NMR (400 MHz, DMSO-d6) δ 7.74 (s, 1H), 7.70-7.57 (m, 2H), 7.49-7.41 (m, 2H), 4.78-4.70 (m, 1H), 4.69-4.61 (m, 1H), 4.08 (d, J=13.3 Hz, 1H), 3.80 (s, 3H), 3.63 (d, J=13.3 Hz, 1H), 3.50 (s, 3H), 3.20 (t, J=11.8 Hz, 1H), 2.43 (s, 3H), 0.95-0.64 (m, 3H), 0.63-0.48 (m, 1H).

LCMS Condition H: Rt=2.87 min. m/z 496.3 [M+H]⁺.

Example 108B (Peak 2): 1H NMR (400 MHz, DMSO-d6) δ 7.77-7.63 (m, 2H), 7.53-7.47 (m, 1H), 7.46-7.37 (m, 2H), 4.79 (d, J=12.8 Hz, 1H), 4.69 (d, J=10.4 Hz, 1H), 4.13 (d, J=12.8 Hz, 1H), 3.81 (s, 3H), 3.66 (d, J=14.0 Hz, 1H), 3.52 (s, 3H), 3.29-3.18 (m, 1H), 2.45 (s, 3H), 0.95-0.67 (m, 3H), 0.66-0.53 (m, 1H).

LCMS Condition H: Rt=2.87 min. m/z 496.3 [M+H]⁺.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=8.45 min for Peak 1 and Rt=9.73 min for Peak 2.

### Example 109A and Example 109B

### (R)-8-(4-chloro-2-fluorophenyl)-2,3-dimethyl-6-(6-(1-methyl-1H-pyrazol-4-yl)-5-oxa-8-azaspiro[3.5]nonan-8-yl)pyrimido[5,4-d]pyrimidin-4 (3H)-one and (S)-8-(4-chloro-2-fluorophenyl)-2,3-dimethyl-6-(6-(1-methyl-1H-pyrazol-4-yl)-5-oxa-8-azaspiro[3.5]nonan-8-yl)pyrimido[5,4-d]pyrimidin-4 (3H)-one

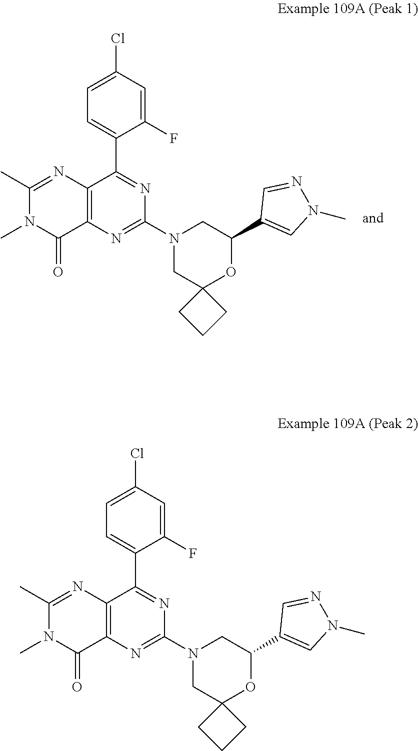

Chiral separation of 8-(4-chloro-2-fluorophenyl)-2,3-dimethyl-6-(6-(1-methyl-1H-pyrazol-4-yl)-5-oxa-8-azaspiro[3.5]nonan-8-yl)pyrimido[5,4-d]pyrimidin-4 (3H)-one (Example 90, 150 mg) was done by normal phase chiral prep HPLC to afford Example 109A, (R)-8-(4-chloro-2-fluorophenyl)-2,3-dimethyl-6-(6-(1-methyl-1H-pyrazol-4-yl)-5-oxa-8-azaspiro[3.5]nonan-8-yl)pyrimido[5,4-d]pyrimidin-4 (3H)-one (38.9 mg, 26.5%; eluted first, assigned as Peak 1 with arbitrary assignment of stereochemistry) as yellow solid and Example 109B, (S)-8-(4-chloro-2-fluorophenyl)-2,3-dimethyl-6-(6-(1-methyl-1H-pyrazol-4-yl)-5-oxa-8-azaspiro[3.5]nonan-8-yl)pyrimido[5,4-d]pyrimidin-4 (3H)-one (50.8 mg, 33.8% yield, eluted later, assigned as Peak 2 with arbitrary assignment of stereochemistry) as yellow solid.

Chiral prep HPLC method details to separate the enantiomers:

Instrument: Agilent 1200 series instrument/Column: CHIRALPAK IG (250×21 mm) 5u/Flow rate: 21.0 ml/min./Mobile phase: Hexane/Dichloromethane/Ethanol: 60/20/20/Solubility: Mixture of Acetonitrile and Dichloromethane/Wave length: 296 nm/Run time: 20 min

Example 109A (Peak 1): 1H NMR (400 MHz, DMSO-d6) δ 7.74 (s, 1H), 7.67 (t, J=8.0 Hz, 1H), 7.62 (d, J=9.9 Hz, 1H), 7.47 (d, J=5.8 Hz, 2H), 4.81 (d, J=13.0 Hz, 1H), 4.72-4.58 (m, 1H), 4.53 (d, J=10.0 Hz, 1H), 3.81 (s, 3H), 3.50 (s, 3H), 3.08-2.91 (m, 2H), 2.43 (s, 3H), 2.12-1.90 (m, 4H), 1.89-1.58 (m, 2H).

LCMS Condition H: Rt=2.92 min. m/z 510.4 [M+H]⁺.

Example 109B (Peak 2) 1H NMR (400 MHz, DMSO-d6) δ 7.74-7.65 (m, 2H), 7.50 (d, J=10.0 Hz, 1H), 7.46-7.38 (m, 2H), 4.83 (d, J=13.2 Hz, 1H), 4.69 (d, J=12.8 Hz, 1H), 4.58 (d, J=10.8 Hz, 1H), 3.82 (s, 3H), 3.53 (s, 3H), 3.12-3.00 (m, 2H), 2.45 (s, 3H), 2.15-1.94 (m, 4H), 1.92-1.62 (m, 2H).

LCMS Condition H: Rt=2.95 min. m/z 510.4 [M+H]⁺.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=6.90 min for Peak 1 and Rt=9.53 min for Peak 2.

### Example 110A and Example 110B

### (R)-8-(4-chloro-2-fluorophenyl)-6-(6-(1-cyclopropyl-1H-pyrazol-4-yl)-2,2-dimethylmorpholino)-2,3-dimethylpyrimido[5,4-d]pyrimidin-4 (3H)-one and (S)-8-(4-chloro-2-fluorophenyl)-6-(6-(1-cyclopropyl-1H-pyrazol-4-yl)-2,2-dimethylmorpholino)-2,3-dimethylpyrimido[5,4-d]pyrimidin-4 (3H)-one

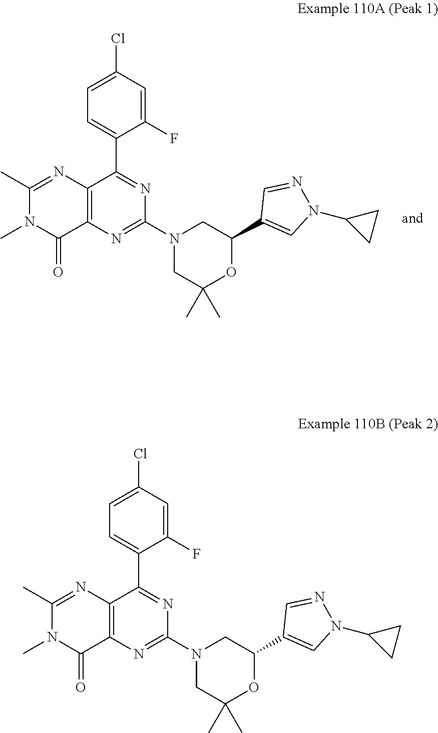 [SMILES: Cc1nc2c(-c3ccc(Cl)cc3F)nc(N3C[C@@H](c4cnn(C5CC5)c4)OC(C)(C)C3)nc2c(=O)n1C ; Cc1nc2c(-c3ccc(Cl)cc3F)nc(N3C[C@H](c4cnn(C5CC5)c4)OC(C)(C)C3)nc2c(=O)n1C]

Chiral separation of 8-(4-chloro-2-fluorophenyl)-6-(6-(1-cyclopropyl-1H-pyrazol-4-yl)-2,2-dimethylmorpholino)-2,3-dimethylpyrimido[5,4-d]pyrimidin-4 (3H)-one (Synthesized by similar protocol as described for Examples 19 and 62, 200 mg) was done by normal phase chiral prep HPLC to afford Example 110A, (R)-8-(4-chloro-2-fluorophenyl)-6-(6-(1-cyclopropyl-1H-pyrazol-4-yl)-2,2-dimethylmorpholino)-2,3-dimethylpyrimido[5,4-d]pyrimidin-4 (3H)-one (82 mg, 41%; eluted first, assigned as Peak 1 with arbitrary assignment of stereochemistry) as yellow solid and Example 1101B, (S)-8-(4-chloro-2-fluorophenyl)-6-(6-(1-cyclopropyl-1H-pyrazol-4-yl)-2,2-dimethylmorpholino)-2,3-dimethylpyrimido[5,4-d]pyrimidin-4 (3H)-one (74 mg, 37% yield, eluted later, assigned as Peak 2 with arbitrary assignment of stereochemistry) as yellow solid.

Chiral prep HPLC method details to separate the enantiomers: Instrument: Agilent 1200 series instrument/Column: CHIRALPAK IC (250×21 mm) 5u/Flow rate: 21.0 ml/min./Mobile phase: Hexane/Dichloromethane/Ethanol: 80/10/10/Solubility: Mixture of Acetonitrile and Dichloromethane/Wave length: 250 nm/Run time: 25 min

Example 110A (Peak 1): 1H NMR (400 MHz, DMSO-d6) δ 7.81 (s, 1H), 7.67-7.59 (m, 2H), 7.45 (d, J=9.6 Hz, 2H), 4.78-4.67 (m, 2H), 4.53 (d, J=12.7 Hz, 1H), 3.74-3.63 (m, 1H), 3.55 (s, 3H), 3.05-2.90 (m, 2H), 2.43 (s, 3H), 1.23 (s, 6H), 1.11-0.99 (m, 2H), 1.03-0.90 (m, 2H).

LCMS Condition P: Rt=2.91 min. m/z 524.2 [M+H]⁺.

Example 110B (Peak 2) 1H NMR (400 MHz, DMSO-d6) δ 7.81 (s, 1H), 7.70-7.57 (m, 2H), 7.45 (d, J=9.6 Hz, 2H), 4.81-4.67 (m, 2H), 4.53 (d, J=12.7 Hz, 1H), 3.74-3.63 (m, 1H), 3.50 (s, 3H), 3.05-2.88 (m, 2H), 2.43 (s, 3H), 1.23 (s, 6H), 1.11-0.99 (m, 2H), 1.03-0.88 (m, 2H).

LCMS Condition P: Rt=2.91 min. m/z 524.2 [M+H]⁺.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=6.44 min for Peak 1 and Rt=9.54 min for Peak 2.

### Example 111

### Example 111A: Enantiomer 1

### 8-(4-chloro-2-fluorophenyl)-6-((4S,6S)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one

### Example 111B: Enantiomer 2

### 8-(4-chloro-2-fluorophenyl)-6-((4R,6R)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one

### Step-1—Preparation of 1-hydroxy-5-methyl-1-(1-methyl-1H-pyrazol-4-yl)hex-4-en-3-one

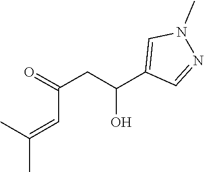

In a two-neck oven-dried round bottom flask, under argon atmosphere mesityl oxide (5.0 g, 50.942 mmol) was dissolved in dry THF (70 mL) and cooled the reaction mixture at −78° C. LDA (2.0 M, 38.5 mL) was slowly added to it and reaction mixture was stirred at −78° C. for one hour in presence of argon atmosphere. A solution of 1-methyl-1H-pyrazole-4-carbaldehyde (4.5 g, 40.754 mmol) in dry THF (30) was slowly added to it and reaction was stirred additional 30 min. After completion, reaction mixture was quenched with saturated ammonium chloride solution and followed by extracted with EtOAc. Combined organic layer was dried over sodium sulphate and concentrated. Purification was performed by column chromatography on silica gel using (30-60) % EtOAc in hexane as eluent to afford title compound 1-hydroxy-5-methyl-1-(1-methyl-1H-pyrazol-4-yl)hex-4-en-3-one (5.0 g, 47.2% yield) as yellow oil.

LCMS Condition A: Rt=1.23 min. m/z 209.2 [M+H]⁺.

1H NMR (400 MHz, DMSO D6) δ 7.51 (s, 1H), 7.29 (s, 1H), 6.18 (s, 1H), 4.97-4.92 (m, 1H), 3.76 (s, 3H), 2.79-2.73 (m, 1H), 2.69-2.63 (m, 1H), 2.04 (s, 3H), 1.84 (s, 3H).

### Step-2—Preparation of 2,2-dimethyl-6-(1-methyl-1H-pyrazol-4-yl)oxan-4-one

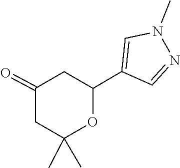

To a stirred solution of 1-hydroxy-5-methyl-1-(1-methyl-1H-pyrazol-4-yl)hex-4-en-3-one (5.0 g, 24.0 mmol) in dry DCM (30 mL) was added Amberlyst-15 (H⁺ form, 20.0 g) and reaction was continued at rt until all the reactant was consumed. After eight hours, reaction mixture was filtered through a pad of celite and washed with DCM several times. Concentration of the solvent under reduced pressure afforded crude as yellow oil. Purification was performed by column chromatography on silica gel using (10-50)% EtOAc in hexane as eluent to afford 2,2-dimethyl-6-(1-methyl-1H-pyrazol-4-yl)oxan-4-one (1.3 g, 26% yield) as yellow oil.

1H NMR (400 MHz, DMSO D6) δ 7.66 (s, 1H), 7.40 (s, 1H), 4.89-4.86 (m, 1H), 3.79 (s, 3H), 2.59 (d, J=12.6 Hz, 1H), 2.368 (d, J=14.04 Hz, 1H), 2.24 (d, J=10.6 Hz, 1H), 1.27 (s, 3H), 1.18 (s, 3H).

### Step-3—Preparation of 2,2-dimethyl-6-(1-methyl-1H-pyrazol-4-yl)-3,6-dihydro-2H-pyran-4-yl 1,1,2,2,3,3,4,4,4-nonafluorobutane-1-sulfonate (mixture)

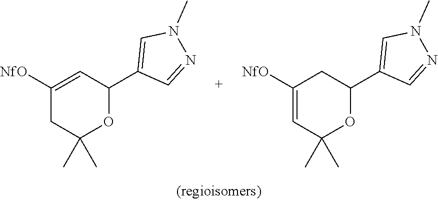

A solution of 2,2-dimethyl-6-(1-methyl-1H-pyrazol-4-yl)oxan-4-one (1.0 g, 4.8 mmol) in dry THF (20 mL) was cooled to 0° C. and followed by DBU (3.6 mL, 24.02 mmol) was added to it. Reaction mixture was stirred for 30 min at 0° C. and followed by Nonafluorobutanesulfonyl fluoride (1.3 mL, 7.205 mmol) was added to it. Reaction mixture was then then stirring for 4 h at rt. After completion, reaction mixture was washed with water and followed by extracted with EtOAc. Combined organic layer was dried over sodium sulfate and followed by concentrated into vacuo to get crude as yellow oil. Purification was performed by column chromatography on silica gel using (0-20)% EtOAc in hexane as eluent to afford 2,2-dimethyl-6-(1-methyl-1H-pyrazol-4-yl)-3,6-dihydro-2H-pyran-4-yl 1,1,2,2,3,3,4,4,4-nonafluorobutane-1-sulfonate (2.0 g, 84.92% yield; Regio isomers) as colourless oil.

1H NMR (400 MHz, DMSO D6) δ 7.87 (s, 1H), 7.71 (s, 1H), 7.41 (s, 1H), 7.33 (s, 1H), 6.13 (bs, 2H), 5.27 (bs, 1H), 4.85-4.82 (m, 1H), 3.8 (s, 6H), 2.62-2.45 (m, 3H), 2.25 (d, J=16.72 Hz, 1H), 1.34 (s, 3H), 1.26 (s, 9H).

### Step-4—Preparation of 4-[6,6-dimethyl-4-(4,4,5,5-tetramethyl-1,3,2-dioxaborolan-2-yl)-5,6-dihydro-2H-pyran-2-yl]-1-methyl-1H-pyrazole (Mixture)

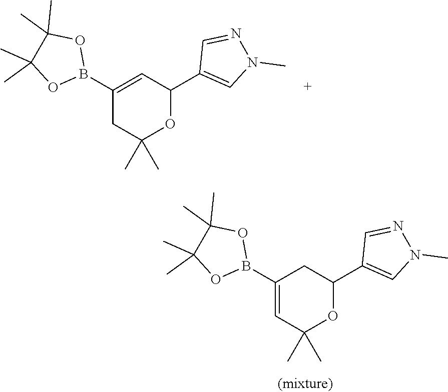

In an oven-dried two neck round bottom flask under inert atmosphere, 2,2-dimethyl-6-(1-methyl-1H-pyrazol-4-yl)-3,6-dihydro-2H-pyran-4-yl 1,1,2,2,3,3,4,4,4-nonafluorobutane-1-sulfonate (2.0 g, 4.08 mmol) was taken and dissolved in 1,4-dioxane (40 mL). Degassed the solution for 5 min under argon atmosphere. Bis(pinacolato)diboron (1.554 g, 6.12 mmol) and potassium acetate (1.6 g, 16.32 mmol) were added into the reaction mass and again degassed for 5 min. PdCl₂(dppf)₂·DCM complex (0.333 g, 0.41 mmol) was added and stirred it for 3 hours at 90° C. After completion, reaction mixture was filtered through celite bed and filtrated portion was dried under reduced pressure. Crude mass was washed with Heptane/Ether (1:1; v/v) and finally dried to get crude 4-[6,6-dimethyl-4-(4,4,5,5-tetramethyl-1,3,2-dioxaborolan-2-yl)-5,6-dihydro-2H-pyran-2-yl]-1-methyl-1H-pyrazole (1.1 g, 84.72 yield) as yellow oil.

NB: This material was forwarded for the next step without further purification to check the feasibility of next step Suzuki reaction and product mass was observed.

LCMS Condition (ELSD): Rt=3.39 min. m/z 318.1 [M+H]⁺.

### Step-5—Preparation of 8-(4-chloro-2-fluorophenyl)-6-[2,2-dimethyl-6-(1-methyl-1H-pyrazol-4-yl)-3,6-dihydro-2H-pyran-4-yl]-2,3-dimethyl-3H,4H-[1,3]diazino[5,4-d]pyrimidin-4-one

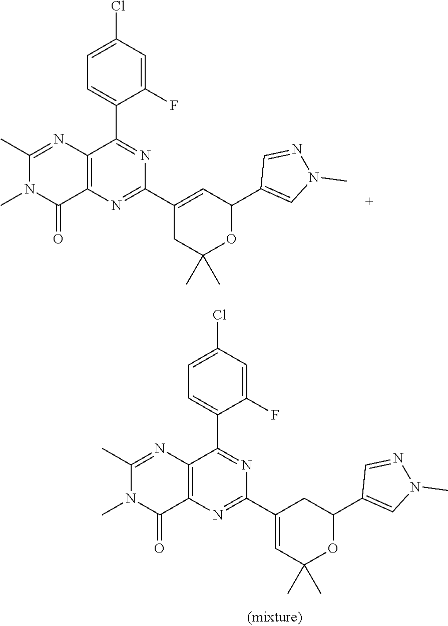

To a suspension of 6-chloro-8-(4-chloro-2-fluorophenyl)-2,3-dimethylpyrimido[5,4-d]pyrimidin-4 (3H)-one (1.0 g, 2.95 mmol) and 4-[6,6-dimethyl-4-(4,4,5,5-tetramethyl-1,3,2-dioxaborolan-2-yl)-5,6-dihydro-2H-pyran-2-yl]-1-methyl-1H-pyrazole (1.4 g, 4.42 mmol) in a mixture of 1,4-dioxane/water (20 mL; 4:1) was added cesium carbonate (2.0 g, 5.9 mmol) and purged the reaction mixture with argon for 5 min. PdCl₂(dppDf)·DCM complex (0.24 g, 0.295 mmol) was then added to it under inert atmosphere and finally it was heated for 4 h at 90° C. After completion, reaction mixture was passed through celite and washed with EtOAc. Concentration of the solvent afforded crude which was washed with water and followed by extracted with water. Organic layer was dried over sodium sulfate and concentrated into vacuo. Purification was performed by column chromatography on silica gel (100-200 mesh size) using (30-80) % EtOAc in hexane as eluent to afford 8-(4-chloro-2-fluorophenyl)-6-[2,2-dimethyl-6-(1-methyl-1H-pyrazol-4-yl)-3,6-dihydro-2H-pyran-4-yl]-2,3-dimethyl-3H,4H-[1,3]diazino[5,4-d]pyrimidin-4-one (0.75 g, 51.44% yield) as white solid.

LCMS Condition A: Rt=1.78 min. m/z 495.2 [M+H]⁺.

### Step-6—Preparation of 8-(4-chloro-2-fluorophenyl)-6-[2,2-dimethyl-6-(1-methyl-1H-pyrazol-4-yl)oxan-4-yl]-2,3-dimethyl-3H,4H-[1,3]diazino[5,4-d]pyrimidin-4-one

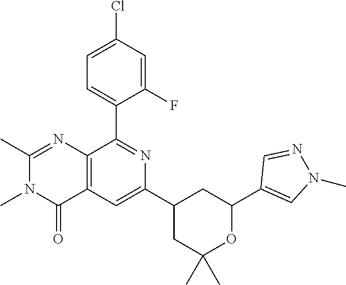

To a solution of 8-(4-chloro-2-fluorophenyl)-6-[2,2-dimethyl-6-(1-methyl-1H-pyrazol-4-yl)-3,6-dihydro-2H-pyran-4-yl]-2,3-dimethyl-3H,4H-[1,3]diazino[5,4-d]pyrimidin-4-one (600.0 mg, 1.2 mmol) in THF (50 mL) were added sodium acetate (298.60 mg, 3.64 mmol) and acetic acid (0.208 mL, 3.64 mmol) and purged the reaction mixture with argon. Pd(OH)2 (150.0 mg, 20% wt) was added to it and mixture was hydrogenated under balloon pressure until all the reactant was consumed. LCMS of the crude reaction mixture after 5 h showed desired product mass (Poor profile; ~10-12%) along with de-halogenated reduced and non-reduced product mass of the reactant as major. After completion, reaction mixture was passed through celite and washed with ethyl acetate. Concentration of the solvent under reduced pressure afforded crude which was washed with aq. NaHCO₃ solution and followed by extracted with DCM. Concentration of the solvent under reduced pressure afforded desired product as yellow solid. Purification was performed by reverse phase prep-HPLC to get 8-(4-chloro-2-fluorophenyl)-6-[2,2-dimethyl-6-(1-methyl-1H-pyrazol-4-yl)oxan-4-yl]-2,3-dimethyl-3H,4H-[1,3]diazino[5,4-d]pyrimidin-4-one (60.0 mg, 10% yield) as white solid.

LCMS Condition B: Rt=2.85 min. m/z 497.37 [M+H]⁺.

### Example 111A and Example 111B

### 8-(4-chloro-2-fluorophenyl)-6-((4S,6S)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one and 8-(4-chloro-2-fluorophenyl)-6-((4R,6R)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one

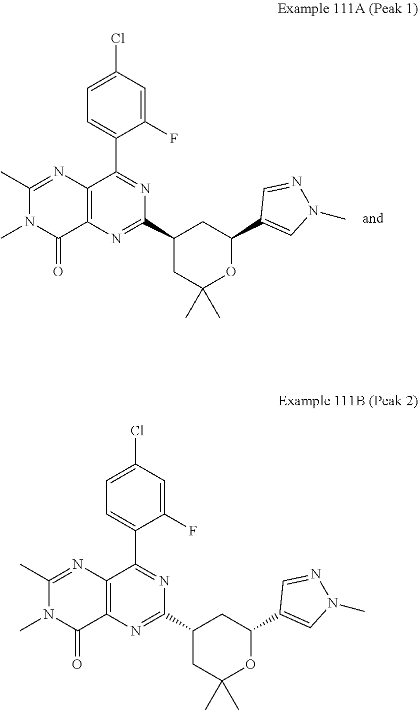

Chiral separation of 8-(4-chloro-2-fluorophenyl)-6-[2,2-dimethyl-6-(1-methyl-1H-pyrazol-4-yl)oxan-4-yl]-2,3-dimethyl-3H,4H-[1,3]diazino[5,4-d]pyrimidin-4-one (50 mg) was done by normal phase chiral prep HPLC to afford Example 111A 8-(4-chloro-2-fluorophenyl)-6-((4S,6S)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one (15 mg, 30%; eluted first, assigned as Peak 1 with arbitrary assignment of stereochemistry) as white solid. and Example 111B, 8-(4-chloro-2-fluorophenyl)-6-((4R,6R)-2,2-dimethyl-6-(1-methyl-1H-pyrazol-4-yl)tetrahydro-2H-pyran-4-yl)-2,3-dimethylpyrimido[5,4-d]pyrimidin-4 (3H)-one (11 mg, 22% yield, eluted later, assigned as Peak 2 with arbitrary assignment of stereochemistry) as white solid.

Chiral prep HPLC method details to separate the enantiomers:

Instrument: Agilent 1200 series instrument/Column: CHIRALPAK IG (250×21 mm) 5u/Flow rate: 21.0 ml/min./Mobile phase: Hexane/Dichloromethane/Ethanol: 80/10/10/Solubility: Mixture of Acetonitrile and Dichloromethane/Wave length: 350 nm/Run time: 36 min

Example 111A (Peak 1): 1H NMR (400 MHz, DMSO-d6) δ 7.71-7.62 (m, 2H), 7.59 (s, 1H), 7.50-7.48 (m, 1H), 7.33 (s, 1H), 4.78 (d, J=10.2 Hz, 1H), 3.77 (s, 3H), 3.59-3.55 (m, 1H), 3.55 (s, 3H), 2.48 (s, 3H), 2.14 (d, J=12.8 Hz, 1H), 1.92 (d, J=12.8 Hz, 1H), 1.75-1.68 (m, 2H), 1.37 (s, 3H), 1.23 (s, 3H).

LCMS Condition H: Rt=2.82 min. m/z 497.3 [M+H]⁺.

Example 111B (Peak 2) 1H NMR (400 MHz, DMSO-d6) δ 7.71-7.62 (m, 2H), 7.59 (s, 1H), 7.50-7.48 (m, 1H), 7.33 (s, 1H), 4.78 (d, J=10.2 Hz, 1H), 3.77 (s, 3H), 3.59-3.55 (m, 1H), 3.55 (s, 3H), 2.48 (s, 3H), 2.14 (d, J=12.8 Hz, 1H), 1.92 (d, J=12.8 Hz, 1H), 1.78-1.68 (m, 2H), 1.37 (s, 3H), 1.24 (s, 3H).

LCMS Condition H: Rt=2.82 min. m/z 497.3 [M+H]⁺.

Analytical chiral HPLS: Chiralpak IC (4.6×250 mm), 5μ Mobile Phase: Hexane/DCM/EtOH/IPAmine: 60/20/20/0.1 Flow Rate: 1.0 ml/min. Solubility: MeOH, Rt=7.59 min for Peak 1 and Rt=8.74 min for Peak 2.

### Example 112

### Example 112A and Example 112B: (R)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one and (S) (R)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one

### Step-1—Preparation of 6-chloro-8-(4-chloro-2-fluorophenyl)-2,3-dimethylpyrido[3,2-d]pyrimidin-4 (3H)-one

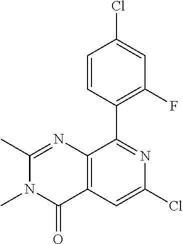 [SMILES: Cc1nc2c(-c3ccc(Cl)cc3F)nc(Cl)cc2c(=O)n1C]

To a stirred solution of 8-bromo-6-chloro-2,3-dimethylpyrido[3,2-d]pyrimidin-4 (3H)-one (100.0 mg, 0.35 mmol) and (4-chloro-2-fluorophenyl)boronic acid (54.47 mg, 0.31 mmol) in dioxane (6 mL) and water (2 mL) was added sodium carbonate (73.54 mg, 0.69 mmol) and degassed with argon. PdCl2(dppf) (25.38 mg, 0.035 mmol) was added under inert atmosphere. The resulting mixture was heated at 80° C. for 1 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (35% ethyl acetate-hexane) to afford 6-chloro-8-(4-chloro-2-fluorophenyl)-2,3-dimethylpyrido[3,2-d]pyrimidin-4 (3H)-one (60 mg, 51.1% yield) as off white solid.

LCMS Condition G: Rt=2.14 min. m/z 338.3 [M+H]⁺.

### Step-2—Preparation of 8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one

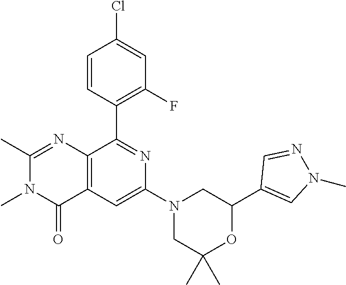

To a stirred solution of 6-chloro-8-(4-chloro-2-fluorophenyl)-2,3-dimethylpyrido[3,2-d]pyrimidin-4 (3H)-one (65.0 mg, 0.19 mmol) and 2,2-dimethyl-6-(1-methyl-1H-pyrazol-4-yl)morpholine (60.1 mg, 0.31 mmol) in toluene (2 mL) degassed with argon was added sodium tertiary butoxide (36.95 mg, 0.39 mmol) and RuPhos (8.97 mg, 0.02 mmol) and RuPhos-Pd-G3 (16.1 mg, 0.02 mmol). The resulting mixture was heated at 100° C. for 12 h. Reaction mixture was diluted with DCM, filtered through a short pad of celite and washed with ethyl acetate. Combined organic part was washed with water, brine, dried over anhydrous sodium sulphate, filtered and concentrated under reduced pressure. Crude was purified by Prep-HPLC to afford 8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one (30 mg, 31.4% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 40% A and 60% B in 3 min, then to 20% A and 80% B in 20 min., then to 5% A and 95% B in 21 min., held this composition up to 22 min. for column washing, then returned to initial composition in 23 min. and held till 25 min.

1H NMR (400 MHz, DMSO-d6) δ 7.70 (s, 1H), 7.55-7.51 (m, 2H), 7.45-7.38 (m, 2H), 7.31 (s, 1H), 4.84-4.80 (m, 1H), 4.37 (d, J=12.0 Hz, 1H), 4.34 (d, J=12.0 Hz, 1H), 3.79 (s, 3H), 3.51 (s, 3H), 2.81-2.75 (m, 2H), 2.43 (s, 3H), 1.28 (s, 3H), 1.24 (s, 3H).

LCMS Condition H: Rt=3.01 min. m/z 497.4 [M+H]⁺.

### Example 112A and Example 112B: (R)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one and (S) (R)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one

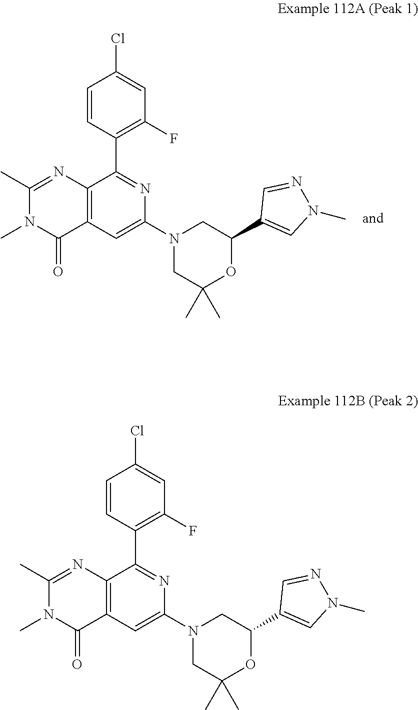

Chiral separation of 8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one (30 mg) was done by normal phase chiral prep HPLC to afford Example 112A, (R)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one (11 mg, 33%; eluted first, assigned as Peak 1 with arbitrary assignment of stereochemistry) as yellow solid and Example 112B, (S)-8-(4-chloro-2-fluorophenyl)-6-(2,2-dimethyl-6-(1-methyl-1H-pyrazol-4-yl)morpholino)-2,3-dimethylpyrido[3,4-d]pyrimidin-4 (3H)-one (11 mg, 33% yield, eluted later, assigned as Peak 2 with arbitrary assignment of stereochemistry) as yellow solid.

Chiral prep HPLC method details to separate the enantiomers:

Instrument: Agilent 1200 series instrument/Column: CHIRALPAK IG (250×21 mm) 5u/Flow rate: 21.0 ml/min./Mobile phase: Hexane/Dichloromethane/Ethanol: 70/15/15/Solubility: Mixture of Acetonitrile and Dichloromethane/Wave length: 308 nm/Run time: 30 min Example 112A (Peak 1): 1H NMR (400 MHz, DMSO-d6) δ 7.70 (s, 1H), 7.55-7.51 (m, 2H), 7.45-7.38 (m, 2H), 7.31 (s, 1H), 4.84-4.80 (m, 1H), 4.37 (d, J=12.0 Hz, 1H), 4.34 (d, J=12.0 Hz, 1H), 3.79 (s, 3H), 3.51 (s, 3H), 2.81-2.75 (m, 2H), 2.43 (s, 3H), 1.28 (s, 3H), 1.24 (s, 3H).

LCMS Condition H: Rt=3.01 min. m/z 497.4 [M+H]⁺.

Example 112B (Peak 2): 1H NMR (400 MHz, DMSO-d6) δ 7.70 (s, 1H), 7.57-7.51 (m, 2H), 7.45-7.38 (m, 2H), 7.31 (s, 1H), 4.84-4.81 (m, 1H), 4.37 (d, J=12.0 Hz, 1H), 4.34 (d, J=12.0 Hz, 1H), 3.79 (s, 3H), 3.51 (s, 3H), 2.81-2.75 (m, 2H), 2.43 (s, 3H), 1.28 (s, 3H), 1.24 (s, 3H).

LCMS Condition H: Rt=3.01 min. m/z 497.4 [M+H]⁺.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=5.84 min for Peak 1 and Rt=7.99 min for Peak 2.

### Example 113

### (S)-2,3-dimethyl-6-(2-(1-methyl-1H-pyrazol-4-yl)morpholino)-8-(3-(trifluoromethyl)bicyclo[1.1.1]pentan-1-yl)pyrimido[5,4-d]pyrimidin-4 (3H)-one

### Step-1—Preparation of 6-chloro-2,3-dimethyl-8-(3-(trifluoromethyl)bicyclo[1.1.1]pentan-1-yl)pyrimido[5,4-d]pyrimidin-4 (3H)-one

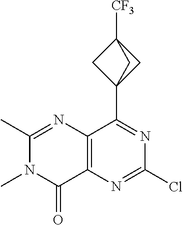 [SMILES: Cc1nc2c(C34CC(C(F)(F)F)(C3)C4)nc(Cl)nc2c(=O)n1C]

Procedure of Step-3, Example 72 was followed using 6,8-dichloro-2,3-dimethylpyrimido[5,4-d]pyrimidin-4 (3H)-one and 3-(trifluoromethyl)bicyclo[1.1.1]pentane-1-carboxylic acid as starting materials to afford 6-chloro-2,3-dimethyl-8-(3-(trifluoromethyl)bicyclo[1.1.1]pentan-1-yl)pyrimido[5,4-d]pyrimidin-4 (3H)-one as off-white solid in 6.3% yield.

LCMS Condition K: Rt=2.32 min. m/z 345.3 [M+H]+.

### Step-2—Preparation of (S)-2,3-dimethyl-6-(2-(1-methyl-1H-pyrazol-4-yl)morpholino)-8-(3-(trifluoromethyl)bicyclo[1.1.1]pentan-1-yl)pyrimido[5,4-d]pyrimidin-4 (3H)-one [Example 113]

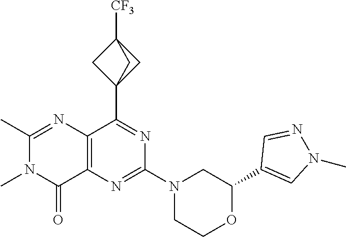

Procedure of Step-5, Example 1 was followed using 6-chloro-2,3-dimethyl-8-(3-(trifluoromethyl)bicyclo[1.1.1]pentan-1-yl)pyrimido[5,4-d]pyrimidin-4 (3H)-one and (S)-2-(1-methyl-1H-pyrazol-4-yl)morpholine as starting materials to afford (S)-2,3-dimethyl-6-(2-(1-methyl-1H-pyrazol-4-yl)morpholino)-8-(3-(trifluoromethyl)bicyclo[1.1.1]pentan-1-yl)pyrimido[5,4-d]pyrimidin-4 (3H)-one as off-white solid in 28.9% yield.

1H NMR (400 MHz, DMSO-d6) δ 7.69 (s, 1H), 7.56 (s, 1H), 4.94 (m, 1H), 4.73-4.69 (m, 1H), 4.58-4.55 (m, 1H), 4.07-4.04 (m, 1H), 3.91 (s, 3H), 3.78-3.71 (m, 1H), 3.64 (s, 3H), 3.28-3.13 (m, 2H), 2.62 (s, 3H), 2.55 (s, 6H).

LCMS Condition L: Rt=2.93 min. m/z 476.4 [M+H]+.

### Example 114

### 4-(4-chloro-2-fluorophenyl)-6,7-dimethyl-2-(2-(1-methyl-1H-pyrazol-4-yl)morpholino)pyrido[3,4-d]pyrimidin-8 (7H)-one

### Step-1—Preparation of methyl 5-iodo-2,6-dioxo-1,2,3,6-tetrahydropyrimidine-4-carboxylate

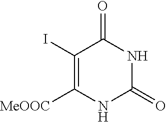 [SMILES: COC(=O)c1[nH]c(=O)[nH]c(=O)c1I]

To a stirred solution of methyl 2,6-dioxo-1,2,3,6-tetrahydropyrimidine-4-carboxylate (5.0 gm, 29.41 mmol) in MeOH (50 mL) were added Iodine (1.5 gm, 5.88 mmol) and Periodic acid (3.4 gm, 17.64 mmol). Resulting mixture was heated at 70° C. for 16 h. Reaction mixture was cooled to RT and concentrated under reduced pressure. The solid residue was suspended in water, collected by filtration, washed well with water and dried under vacuum to afford methyl 5-iodo-2,6-dioxo-1,2,3,6-tetrahydropyrimidine-4-carboxylate (6 gm, yield 68.93%) as off-white solid.

LCMS Condition L: Rt=0.98 min. m/z 297.2 [M+H]+.

### Step-2—Preparation of methyl 5-iodo-1,3-bis(4-methoxybenzyl)-2,6-dioxo-1,2,3,6-tetrahydropyrimidine-4-carboxylate

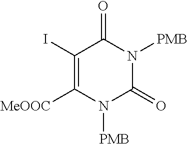

To a stirred solution of methyl 5-iodo-2,6-dioxo-1,2,3,6-tetrahydropyrimidine-4-carboxylate (6.5 gm, 21.96 mmol) in DMF (50.0 mL), was added Na2CO3 (6.98 g, 65.88 mmol) and stirred for 10 min at RT. PMB-chloride (6.0 mL, 43.92 mmol) was then added dropwise to the reaction mixture. Resulting mixture was stirred at RT for 16 h. Reaction mixture was quenched by cool water and extracted with ethyl acetate. Combined organic layer was dried over anhydrous sodium sulphate, filtered and concentrated under reduced pressure. Crude mass was purified by column chromatography (20-30% ethyl acetate-hexane) to afford methyl 5-iodo-1,3-bis(4-methoxybenzyl)-2,6-dioxo-1,2,3,6-tetrahydropyrimidine-4-carboxylate (8.5 gm, yield 72.17%) as an off white solid.

LCMS Condition K: Rt=2.31 min. m/z 537.3 [M+H]+.

### Step-3—Preparation of methyl 1,3-bis(4-methoxybenzyl)-2,6-dioxo-5-(prop-1-yn-1-yl)-1,2,3,6-tetrahydropyrimidine-4-carboxylate

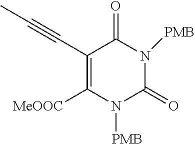

To a stirred solution of methyl 5-iodo-1,3-bis(4-methoxybenzyl)-2,6-dioxo-1,2,3,6-tetrahydropyrimidine-4-carboxylate (3.5 g, 6.53 mmol) in THF (40 mL) and was added triethyl amine (2.0 ml, 13.06 mmol) and degassed with argon. CuI (250 mg, 1.31 mmol) and PdCl₂(PPh3)2 (230 mg, 0.05 mmol) were added under inert atmosphere. Reaction mixture was then cooled to 0° C. and propyne gas was purged for 2 min Resulting mixture was stirred at RT for 48 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (20-30% ethyl acetate in hexane) to afford methyl 1,3-bis(4-methoxybenzyl)-2,6-dioxo-5-(prop-1-yn-1-yl)-1,2,3,6-tetrahydropyrimidine-4-carboxylate (3.0 gm, 99.03%) as yellow solid.

LCMS Condition K: Rt=2.34 min. m/z 449.2 [M+H]+.

### Step-4—Preparation of 1,3-bis(4-methoxybenzyl)-2,6-dioxo-5-(prop-1-yn-1-yl)-1,2,3,6-tetrahydropyrimidine-4-carboxylic acid

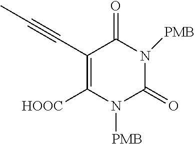

To a stirred solution of methyl 1,3-bis(4-methoxybenzyl)-2,6-dioxo-5-(prop-1-yn-1-yl)-1,2,3,6-tetrahydropyrimidine-4-carboxylate (2.5 g, 5.58 mmol) in THF (40 mL) and methanol (10 mL) was added NaOH (669.64 mg, 16.74 mmol). Resulting mixture was stirred at RT for 16 hours. Reaction mixture was diluted with water, washed with ethyl acetate and layers were partitioned. Ethyl acetate portion was discarded and aqueous portion was acidified with 1 N H2SO4 and extracted with DCM. Combined DCM portion was dried over anhydrous sodium sulphate, filtered and concentrated under reduced pressure. to afford 1,3-bis[(4-methoxyphenyl)methyl]-2,6-dioxo-5-(prop-1-yn-1-yl)-1,2,3,6-tetrahydropyrimidine-4-carboxylic acid (1.2 gm, yield 51.56%) as off white solid.

LCMS Condition K: Rt=1.84 min. m/z 435.2 [M+H]+.

### Step-5—Preparation of 1,3-bis(4-methoxybenzyl)-6-methyl-1H-pyrano[3,4-d]pyrimidine-2,4,8 (3H)-trione

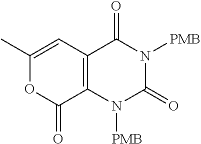

To a stirred solution of 1,3-bis[(4-methoxyphenyl)methyl]-2,6-dioxo-5-(prop-1-yn-1-yl)-1,2,3,6-tetrahydropyrimidine-4-carboxylic acid (1.2 g, 0.27 mmol) in DMF (25 mL) was added AgBF4 (280.82 mg, 1.44 mmol) under argon atmosphere. Resulting mixture was stirred at 80° C. for 16 h. Reaction mixture was diluted with ethyl acetate and washed with water. Organic portion dried over anhydrous sodium sulphate, filtered and concentrated under reduced pressure. Crude mass was purified by combi flash chromatography using 10-20% ethyl acetate in hexane to afford 1,3-bis[(4-methoxyphenyl)methyl]-6-methyl-1H,2H,3H,4H,8H-pyrano[3,4-d]pyrimidine-2,4,8-trione (170 mg, 13.59%) as yellow solid.

LCMS Condition K: Rt=2.34 min. m/z 435.2 [M+H]+.

### Step-6—Preparation of 1,3-bis(4-methoxybenzyl)-6,7-dimethyl-1,7-dihydropyrido[3,4-d]pyrimidine-2,4,8 (3H)-trione

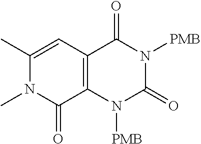

To a stirred solution of 1,3-bis[(4-methoxyphenyl)methyl]-6-methyl-1H,2H,3H,4H,8H-pyrano[3,4-d]pyrimidine-2,4,8-trione (170 mg, 0.391 mmol) in acetic acid (4 mL) in seal tube were added NaOAc (32.11 mg, 0.39 mmol) and MeNH2 [2(M) in THF, 4 ml, 7.827 mmol] at RT. Resulting mixture was stirred at RT for 2 h and then heated at 120° C. for 16 h. Reaction mixture was concentrated under reduced pressure. Crude mass was treated with saturated aqueous NaHCO₃ solution and extracted with ethyl acetate. Organic portion was dried over anhydrous sodium sulphate, filtered and concentrated under reduced pressure. Crude mass was purified by combi flash chromatography using 30-50% ethyl acetate in hexane to afford 1,3-bis(4-methoxybenzyl)-6,7-dimethyl-1,7-dihydropyrido[3,4-d]pyrimidine-2,4,8 (3H)-trione (100 mg, yield 57.1%) as gummy liquid.

LCMS Condition K: Rt=1.94 min. m/z 448.4 [M+H]+.

### Step-7—Preparation of 6,7-dimethyl-1,7-dihydropyrido[3,4-d]pyrimidine-2,4,8 (3H)-trione

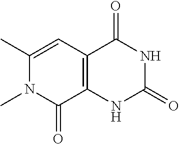

To a stirred solution of 1,3-bis(4-methoxybenzyl)-6,7-dimethyl-1,7-dihydropyrido[3,4-d]pyrimidine-2,4,8 (3H)-trione (100 mg, 0.22 mmol) in TFA (1.5 mL) was added Triflic acid (1.5 mL) at 0° C. Resulting mixture was stirred at RT for 16 h. Reaction mixture was concentrated under reduced pressure. The concentrated mass was treated with saturated aqueous NaHCO₃ solution and extracted with 30% isopropyl alcohol-DCM solution. Combined organic portion was dried over anhydrous sodium sulphate, filtered and concentrated under reduced pressure. Crude mass was purified by combi flash chromatography using 5-10% methanol in DCM to afford 6,7-dimethyl-1H,2H,3H,4H,7H,8H-pyrido[3,4-d]pyrimidine-2,4,8-trione (40 mg, yield 86.3%) as sticky solid.

LCMS Condition K: Rt=0.66 min. m/z 208.2 [M+H]+.

### Step-8—Preparation of 2,4-dichloro-6,7-dimethylpyrido[3,4-d]pyrimidin-8 (7H)-one

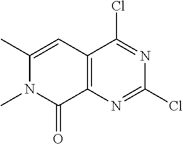

To a stirred solution of 6,7-dimethyl-1H,2H,3H,4H,7H,8H-pyrido[3,4-d]pyrimidine-2,4,8-trione (40 mg, 0.193 mmol) in MeCN (2 mL), was added DIPEA (0.013 ml, 0.077 mmol) and POCl3 (0.108 ml, 1.159 mmol) at 0° C. under argon atmosphere. Resulting mixture was stirred at RT for 15 min and then heated to 100° C. for 8 h. The mixture was cooled to RT and concentrated under reduced pressure. The residue was cooled to 0° C., neutralized with saturated aqueous NaHCO₃ solution and extracted with ethyl acetate. Combined organic part was washed with water, brine, dried over anhydrous sodium sulphate, filtered and concentrated under reduced pressure. Crude mass was purified by column chromatography (50% ethyl acetate-hexane) to afford 2,4-dichloro-6,7-dimethyl-7H,8H-pyrido[3,4-d]pyrimidin-8-one (25 mg, 53.01%) as off white solid.

LCMS Condition K: Rt=1.97 min. m/z 244.3 [M+H]+.

### Step-9—Preparation of 2-chloro-4-(4-chloro-2-fluorophenyl)-6,7-dimethylpyrido[3,4-d]pyrimidin-8 (7H)-one

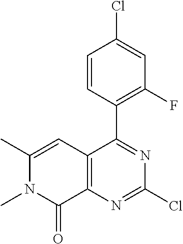 [SMILES: Cc1cc2c(-c3ccc(Cl)cc3F)nc(Cl)nc2c(=O)n1C]

To a stirred solution of 2,4-dichloro-6,7-dimethyl-7H,8H-pyrido[3,4-d]pyrimidin-8-one (25 mg, 0.103 mmol) and (4-chloro-2-fluorophenyl)boronic acid (16.111 mg, 0.093 mmol) dioxane (2 mL) and water (0.5 mL) was added sodium carbonate (21.81 mg, 0.21 mmol) and degassed with argon. PdCl2(dppf) (3.75 mg, 0.005 mmol) was added under inert atmosphere. The resulting mixture was heated at 60° C. for 1 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (50-70% ethyl acetate-hexane) to afford 2-chloro-4-(4-chloro-2-fluorophenyl)-6,7-dimethylpyrido[3,4-d]pyrimidin-8 (7H)-one (18 mg, 51.74%) as off white solid.

LCMS Condition K: Rt=2.09 min. m/z 238.2 [M+H]+.

### Step-10—Preparation of 4-(4-chloro-2-fluorophenyl)-6,7-dimethyl-2-(2-(1-methyl-1H-pyrazol-4-yl)morpholino)pyrido[3,4-d]pyrimidin-8 (7H)-one [Example 114]

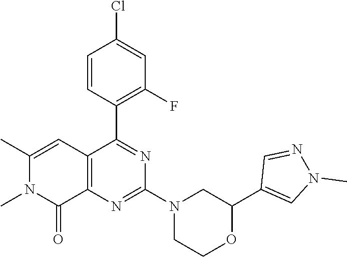

Procedure of Step-5, Example 1 was followed using 2-chloro-4-(4-chloro-2-fluorophenyl)-6,7-dimethylpyrido[3,4-d]pyrimidin-8 (7H)-one and 2-(1-methyl-1H-pyrazol-4-yl)morpholine as starting materials followed by prep HPLC purification to afford 4-(4-chloro-2-fluorophenyl)-6,7-dimethyl-2-(2-(1-methyl-1H-pyrazol-4-yl)morpholino)pyrido[3,4-d]pyrimidin-8 (7H)-one [Example 114]: as white solid in 23.4% yield.

Prep HPLC method used for purification of Example 114:

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 30% A and 70% B in 22 min, then to 5% A and 95% B in 23 min., held this composition up to 25 min. for column washing, then returned to initial composition in 26 min. and held till 28 min.

1H NMR (400 MHz, DMSO) δ 7.74 (s, 1H), 7.70-7.66 (m, 1H), 7.61 (t, J=7.9 Hz, 1H), 7.53-7.48 (m, 1H), 7.45 (s, 1H), 6.09-5.92 (m, 1H), 4.63 (d, J=12.8 Hz, 1H), 4.58-4.46 (m, 2H), 4.00 (d, J=10.2 Hz, 1H), 3.81 (s, 3H), 3.70-3.60 (m, 1H), 3.50 (s, 3H), 3.22-3.07 (m, 2H), 2.31 (s, 3H).

LCMS Condition K: Rt=2.79 min. m/z 469.3 [M+H]+.

### Example 115

### 8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one

### Step-1—Preparation of 1-(1-methyl-1H-pyrazol-4-yl)-2-nitroethan-1-ol

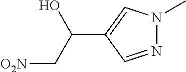

To a stirred solution of 1-methyl-1H-pyrazole-4-carbaldehyde (10 gm, 90.43 mmol) in nitromethane (100.0 mL) at 0° C. triethyl amine (26 mL, 180 mmol) was added to the reaction mixture dropwise. Resulting mixture was stirred at RT for 6 h. The reaction mixture was evaporated and in column chromatography (silica gel, 20-70% ethyl acetate in Hexane) to get 1-(1-methyl-1H-pyrazol-4-yl)-2-nitroethan-1-ol (4.0 g, 25%) as brown gummy mass.

LCMS Condition A: Rt=1.18 min. m/z 172.1 [M+H]+.

### Step-2—Preparation of 2-amino-1-(1-methyl-1H-pyrazol-4-yl)ethan-1-ol

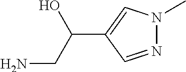

To a stirred solution of 1-(1-methyl-1H-pyrazol-4-yl)-2-nitroethan-1-ol (3.2 gm, 18.7 mmol) in methanol (64.0 mL), glacial acetic acid (0.2 mL) was added. Finally it was degassed with nitrogen and 10% Pd/C (1.6 gm) was added and under vacuum condition it was charged with hydrogen gas under Belone pressure and kept it stirring for 16 h. After completion of reaction it filtered through celite bed and washed with methanol and concentrated to get 2-amino-1-(1-methyl-1H-pyrazol-4-yl)ethan-1-ol (2.4 g, 70%) as gummy liquid.

LCMS Condition A: Rt=0.19 min. m/z 142.1 [M+H]+.

### Step-3—Preparation of 2-bromo-2,2-difluoro-N-(2-hydroxy-2-(1-methyl-1H-pyrazol-4-yl)ethyl)acetamide

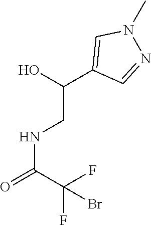 [SMILES: Cn1cc(C(O)CNC(=O)C(F)(F)Br)cn1]

To a stirred solution of 2-amino-1-(1-methyl-1H-pyrazol-4-yl)ethan-1-ol (2.4 g, 17.0 mmol) in DMF (20.0 mL), ethyl-2-bromo-2,2-difluoro acetate (3.43 g, 17.0 mmol) was added. Resulting mixture was stirred at RT for 16 h. Reaction was quenched with crushed ice and extracted with 10% methanol-DCM (100 mL). Combined organic layer was washed with brine, dried over sodium sulphate, filtered and evaporated under reduced pressure. Crude product was purified by combi-flash chromatography (Methanol:DCM, 1:9) to get 2-bromo-2,2-difluoro-N-(2-hydroxy-2-(1-methyl-1H-pyrazol-4-yl)ethyl)acetamide (1.1 g, 31%) as white solid.

LCMS Condition A: Rt=1.80 min. m/z 298.0 [M+H]⁺.

### Step-4—Preparation of 2,2-difluoro-6-(1-methyl-1H-pyrazol-4-yl)morpholin-3-one

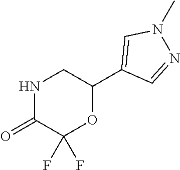

To a stirred solution of 2-bromo-2,2-difluoro-N-(2-hydroxy-2-(1-methyl-1H-pyrazol-4-yl)ethyl)acetamide (1.1 g, 3.69 mmol) in THF (30.0 mL) under ice cold condition, sodium tert butoxide (3.6 mL, 2.0 M in THF, 7.38 mmol) was added. The reaction was stirred at RT for 16 h. Reaction was quenched with crushed ice and extracted with 10% MeOH in DCM (50 mL×3). Combined organic layer was washed with brine, dried over sodium sulphate, filtered and evaporated under reduced pressure. Crude product was purified by combi-flash chromatography (3-4% MeOH-DCM) to have 2,2-difluoro-6-(1-methyl-1H-pyrazol-4-yl)morpholin-3-one (20 mg, ~2%) as white solid.

1H NMR (400 MHz, DMSO-d6) δ 7.76 (s, 1H), 7.56 (s, 1H), 5.49-5.45 (m, 1H), 3.88 (s, 3H), 3.84-3.73 (m, 1H), 3.54-3.51 (m, 1H).

LCMS Condition A: Rt=1.30 min. m/z 218.2 [M+H]⁺.

### Step-5—Preparation of 2,2-difluoro-6-(1-methyl-1H-pyrazol-4-yl)morpholine

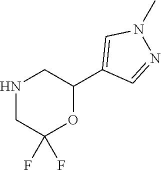

To a stirred solution of 2,2-difluoro-6-(1-methyl-1H-pyrazol-4-yl)morpholin-3-one (20 mg 0.092 mmol) in THF (5.0 mL), BH3-DMS (0.2 mL, 2M in THF, 0.27 mmol) at 0° C. Resulting mixture was heated at 55° C. for 3 h. Reaction was quenched with methanol evaporated the reaction mixture and washed with bicarbonate (5 mL) and extracted with 20% Methanol/DCM (40 mL). Combined organic layer was washed with brine, dried over sodium sulphate, filtered and evaporated under reduced pressure to have 2,2-difluoro-6-(1-methyl-1H-pyrazol-4-yl)morpholine (18 mg crude) as gummy liquid.

LCMS Condition A: Rt=1.57 min. m/z 204.17 [M+H]⁺.

### Step-6—Preparation of 8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one

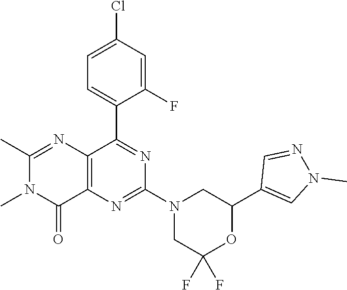

To a stirred solution of 6-chloro-8-(4-chloro-2-fluorophenyl)-2,3-dimethylpyrimido[5,4-d]pyrimidin-4 (3H)-one (30 mg, 0.089 mmol) in DMSO (3 mL), was added 2,2-difluoro-6-(1-methyl-1H-pyrazol-4-yl)morpholine (18 mg, crude) and DIPEA (0.062 ml, 0.35 mmol) at rt. Then the reaction mixture was stirred at 80° C. for 16 h. After completion of reaction, Reaction mixture was diluted with water and extracted with ethyl acetate, organic layer was separated, washed with brine solution and dried over anhydrous sodium sulfate. The filtrate was evaporated under reduced pressure to afford the crude which was purified through prep HPLC to afford 8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one (4 mg, 49.13%) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 40% A and 60% B in 3 min, then to 30% A and 70% B in 20 min., then to 5% A and 95% B in 21 min., held this composition up to 22 min. for column washing, then returned to initial composition in 23 min. and held till 25 min.

1H NMR (400 MHz, DMSO-d6) δ 7.93 (s, 1H), 7.70 (t, J=7.9 Hz, 1H), 7.67-7.57 (m, 2H), 7.52-7.43 (m, 1H), 5.28 (d, J=8.7 Hz, 1H), 4.96 (d, J=9.5 Hz, 1H), 4.73 (d, J=13.6 Hz, 1H), 3.84 (s, 3H), 3.80-3.65 (m, 1H), 3.61-3.42 (m, 4H), 2.46 (s, 3H).

LCMS Condition K: Rt=2.94 min. m/z 506.3 [M+H]⁺.

### Example 116

### 8-(4-chloro-2-fluorophenyl)-6-(2-(1-cyclopropyl-1H-pyrazol-4-yl)-3-oxabicyclo[4.1.0]heptan-6-yl)-2,3-dimethylpyrimido[5,4-d]pyrimidin-4 (3H)-one

### Example 116A: Enantiomer Peak 1: 8-(4-chloro-2-fluorophenyl)-6-((1S,2S,6R)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one

### Example 116B: Enantiomer Peak 2: 8-(4-chloro-2-fluorophenyl)-6-((1R,2R,6S)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one

### Step-1—Preparation of 8-(4-chloro-2-fluorophenyl)-6-(2-(1-cyclopropyl-1H-pyrazol-4-yl)-3-oxabicyclo[4.1.0]heptan-6-yl)-2,3-dimethylpyrimido[5,4-d]pyrimidin-4 (3H)-one

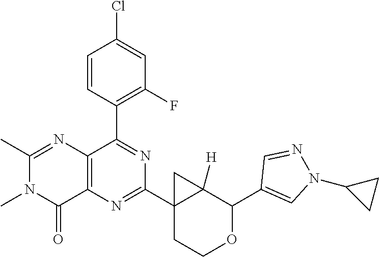 [SMILES: Cc1nc2c(-c3ccc(Cl)cc3F)nc(C34CCOC(c5cnn(C6CC6)c5)C3C4)nc2c(=O)n1C]

To a mixture of sodium hydride (48.78 mg, 1.22 mmol) and trimethyloxosulfonium chloride (196.067 mg, 1.524 mmol) under inert atmosphere was added anhydrous DMSO (2 mL) at RT. Resultant mixture was heated at 65° C. for 30 min. Then a solution 8-(4-chloro-2-fluorophenyl)-6-(6-(1-cyclopropyl-1H-pyrazol-4-yl)-3,6-dihydro-2H-pyran-4-yl)-2,3-dimethylpyrimido[5,4-d]pyrimidin-4 (3H)-one (300 mg, 0.61 mmol) in anhydrous DMSO (2 ml) was added under heating condition and the reaction mixture stirred at 65° C. for 4 h. Reaction mixture was diluted with ethyl acetate and washed with water and brine. dried over anhydrous sodium sulphate, filtered and concentrated under reduced pressure. Crude mass was purified by reverse phase prep HPLC to afford 8-(4-chloro-2-fluorophenyl)-6-(2-(1-cyclopropyl-1H-pyrazol-4-yl)-3-oxabicyclo[4.1.0]heptan-6-yl)-2,3-dimethylpyrimido[5,4-d]pyrimidin-4 (3H)-one (60 mg, 19.4% yield) as white solid.

Preparative HPLC was done on Waters auto purification instrument. Column name: Kinetex evo C18 (250×21.2 mm, 5μ) operating at ambient temperature and flow rate of 16 mL/min. Mobile phase: A=20 mM Ammonium Bicarbonate in water, B=Acetonitrile: Methanol (50:50); Gradient Profile: Mobile phase initial composition of 40% A and 60% B, then 20% A and 90% B in 22 min, then to 5% A and 95% B in 23 min., held this composition up to 25 min. for column washing, then returned to initial composition in 26 min. and held till 28 min.

1H NMR (400 MHz, Chloroform-d) δ 7.60 (t, J=7.8 Hz, 1H), 7.44 (d, J=6.6 Hz, 2H), 7.32-7.19 (m, 2H), 4.89 (s, 1H), 3.80-3.69 (m, 1H), 3.64 (s, 3H), 3.60-3.47 (m, 1H), 3.45-3.34 (m, 1H), 3.24-3.13 (m, 1H), 2.59 (s, 3H), 2.23-2.12 (m, 2H), 1.83-1.75 (m, 1H), 1.36-1.27 (m, 1H), 1.14-1.02 (m, 2H), 1.04-0.90 (m, 2H).

LCMS Condition L: Rt=2.87 min. m/z 507.3 [M+H]+.

### Step-2—Preparation of 8-(4-chloro-2-fluorophenyl)-6-((1S,2S,6R)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one [Example 116A] and 8-(4-chloro-2-fluorophenyl)-6-((1R,2R,6S)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one [Example 116B]

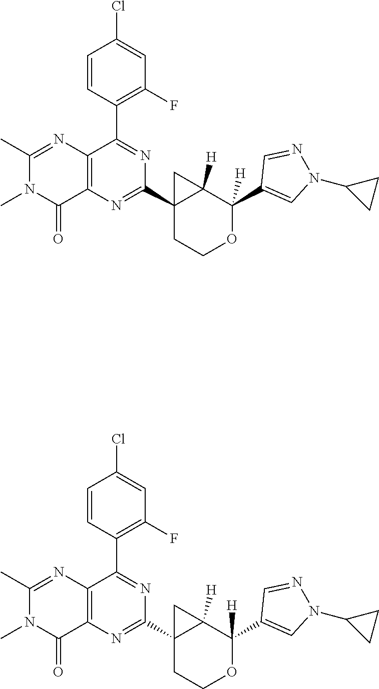 [SMILES: Cc1nc2c(-c3ccc(Cl)cc3F)nc([C@]34CCO[C@H](c5cnn(C6CC6)c5)[C@H]3C4)nc2c(=O)n1C ; Cc1nc2c(-c3ccc(Cl)cc3F)nc([C@@]34CCO[C@@H](c5cnn(C6CC6)c5)[C@@H]3C4)nc2c(=O)n1C]

8-(4-chloro-2-fluorophenyl)-6-((1S,2S,6R)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one [Example 116A, Peak1] and 8-(4-chloro-2-fluorophenyl)-6-((1R,2R,6S)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one [Example 116B, Peak2] were obtained by chiral separation of 8-(4-chloro-2-fluorophenyl)-6-(2-(1-cyclopropyl-1H-pyrazol-4-yl)-3-oxabicyclo[4.1.0]heptan-6-yl)-2,3-dimethylpyrimido[5,4-d]pyrimidin-4 (3H)-one by SFC. Absolute stereochemistry was arbitrarily assigned.

Chiral prep HPLC method details to separate the enantiomers:

Column: I Cellulose J (30 mm×250 mm), 5μ/Flow: 100 ml/min/Mobile Phase: 80% CO2+20% (100% Methanol)/ABPR: 100 bar/Temp: 35° C./UV: 280 nm/DILUENT: Methanol+MeCN/Loading: 1.08 mg/4.2 min/Sample concentration: 9.0 mg/ml Example 116A, Peak1: 1H NMR (400 MHz, Chloroform-d) δ 7.60 (t, J=7.8 Hz, 1H), 7.44 (d, J=6.6 Hz, 2H), 7.32-7.19 (m, 2H), 4.89 (s, 1H), 3.80-3.69 (m, 1H), 3.64 (s, 3H), 3.60-3.47 (m, 1H), 3.45-3.34 (m, 1H), 3.24-3.13 (m, 1H), 2.59 (s, 3H), 2.23-2.12 (m, 2H), 1.83-1.75 (m, 1H), 1.36-1.27 (m, 1H), 1.14-1.02 (m, 2H), 1.04-0.90 (m, 2H).

LCMS Condition L: Rt=2.87 min. m/z 507.3 [M+H]+.

Example 116B, Peak2: 1H NMR (400 MHz, Chloroform-d) δ 7.60 (t, J=7.8 Hz, 1H), 7.44 (d, J=6.6 Hz, 2H), 7.32-7.19 (m, 2H), 4.89 (s, 1H), 3.80-3.69 (m, 1H), 3.64 (s, 3H), 3.60-3.47 (m, 1H), 3.45-3.34 (m, 1H), 3.24-3.13 (m, 1H), 2.59 (s, 3H), 2.23-2.12 (m, 2H), 1.83-1.75 (m, 1H), 1.36-1.27 (m, 1H), 1.14-1.02 (m, 2H), 1.04-0.90 (m, 2H).

LCMS Condition L: Rt=2.87 min. m/z 507.3 [M+H]+.

Analytical chiral HPLC by SFC: I-CELLULOSE J column Mobile Phase: 0.3% IP amine in MEOH Flow Rate: 8.0 ml/min. Solubility: MeOH, Rt=5.26 min for Peak 1 and Rt=6.48 min for Peak 2.

### Example 117

### Example 117A—Peak1

### (R)-8-(4-chloro-2-fluorophenyl)-6-(2,2-dimethyl-6-(1-methyl-1H-pyrazol-4-yl)morpholino)-2,3-dimethylquinazolin-4 (3H)-one

### Example 117B—Peak2

### (S)-8-(4-chloro-2-fluorophenyl)-6-(2,2-dimethyl-6-(1-methyl-1H-pyrazol-4-yl)morpholino)-2,3-dimethylquinazolin-4 (3H)-one

(R)-8-(4-chloro-2-fluorophenyl)-6-(2,2-dimethyl-6-(1-methyl-1H-pyrazol-4-yl)morpholino)-2,3-dimethylquinazolin-4 (3H)-one [Example 117A (Peak1)] and (S)-8-(4-chloro-2-fluorophenyl)-6-(2,2-dimethyl-6-(1-methyl-1H-pyrazol-4-yl)morpholino)-2,3-dimethylquinazolin-4 (3H)-one [Example 117B (Peak2)] were obtained by chiral separation of 8-(4-chloro-2-fluorophenyl)-6-(2,2-dimethyl-6-(1-methyl-1H-pyrazol-4-yl)morpholino)-2,3-dimethylquinazolin-4 (3H)-one by normal phase chiral prep HPLC. Absolute stereochemistry was arbitrarily assigned.

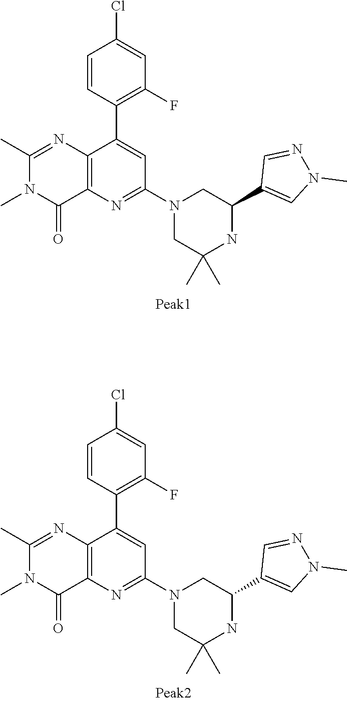

Chiral prep HPLC method details to separate the enantiomers: Instrument: Agilent 1200 series instrument/Column: CHIRALPAK IG (250×30 mm) 5μ/Flow rate: 27.0 mL/min./Mobile phase: Hexane/Dichloromethane/Ethanol: 60/20/20/Solubility: Mixture of Acetonitrile and Dichloromethane/Wave length: 304 nm/Run time: 25 min

Synthetic protocol for preparation of racemate compound [8-(4-chloro-2-fluorophenyl)-6-(2,2-dimethyl-6-(1-methyl-1H-pyrazol-4-yl)morpholino)-2,3-dimethylquinazolin-4 (3H)-one] of Example 117A and Example 117B) is same as for Example 106. Starting materials used for the synthesis of racemate compound are methyl 3-bromo-5-fluoro-2-nitrobenzoate (commercially available) and 2,2-dimethyl-6-(1-methyl-1H-pyrazol-4-yl)morpholine, HCl salt (synthesized in step-5, Example 62).

Example 117A, Peak1: 1H NMR (400 MHz, DMSO-d6) δ 7.70 (s, 1H), 7.57-7.41 (m, 5H), 7.36 (d, J=8.3 Hz, 1H), 4.89 (d, J=9.2 Hz, 1H), 3.89-3.72 (m, 4H), 3.66 (d, J=11.7 Hz, 1H), 3.51 (s, 3H), 2.69-2.56 (m, 2H), 2.41 (s, 3H), 1.38 (s, 3H), 1.23 (s, 3H).

LCMS Condition H: Rt=2.99 min. m/z 496.4 [M+H]+.

Example 117B, Peak2: 1H NMR (400 MHz, DMSO-d6) δ 7.70 (s, 1H), 7.57-7.41 (m, 5H), 7.36 (d, J=8.3 Hz, 1H), 4.89 (d, J=9.2 Hz, 1H), 3.89-3.72 (m, 4H), 3.66 (d, J=11.7 Hz, 1H), 3.51 (s, 3H), 2.69-2.56 (m, 2H), 2.41 (s, 3H), 1.38 (s, 3H), 1.23 (s, 3H).

LCMS Condition H: Rt=2.99 min. m/z 496.4 [M+H]+.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=7.56 min for Peak 1 and Rt=10.12 min for Peak 2.

### Example 118

### Example 118A—Peak1

### 6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one

### Example 118B—Peak2

### 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one

### Step-1—Preparation of 8-bromo-6-chloro-2-methyl-4H-benzo[d][1,3]oxazin-4-one

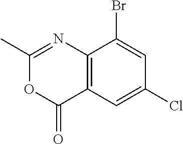

A stirred solution of 2-amino-3-bromo-5-chloro-benzoic acid (2.0 gm, 8 mmol) in Acetic Anhydride (20.0 ml) in a sealed tube was heated at 100° C. for 1 h. The reaction mixture was quenched with ice cold water and a yellow precipitate was formed which was collected by filtration and dried under reduced pressure to afford 8-bromo-6-chloro-2-methyl-4H-benzo[d][1,3]oxazin-4-one (2.0 gm, yield 91.08%) as an off white solid.

LCMS Condition K: Rt=2.21 min. m/z 274.2 [M+H]+.

### Step-2—Preparation of 8-bromo-6-chloro-2,3-dimethylquinazolin-4 (3H)-one

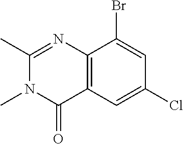

To a stirred solution of 8-bromo-6-chloro-2-methyl-4H-benzo[d][1,3]oxazin-4-one (2 g, 7.25 mmol) in THF (5.0 mL) was added MeNH2 in THF [2(M), 21.7 ml, 43.44 mmol] at 10° C. Resulting mixture was heated at 100° C. for 16 h. Reaction mixture was concentrated under reduced pressure. The crude mass was purified by column chromatography (20%-50% ethyl acetate-hexane) to afford 8-bromo-6-chloro-2,3-dimethylquinazolin-4 (3H)-one (800 mg, yield 62.4%) as a white solid.

LCMS Condition K: Rt=1.56 min. m/z 287.2 [M+H]+.

### Step-3—Preparation of 6-chloro-8-(2-fluoro-4-(trifluoromethyl)phenyl)-2,3-dimethylquinazolin-4 (3H)-one

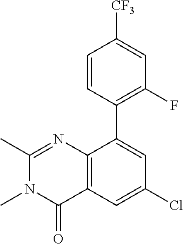 [SMILES: Cc1nc2c(-c3ccc(C(F)(F)F)cc3F)cc(Cl)cc2c(=O)n1C]

Procedure of Step-9, Example 114 was followed using 8-bromo-6-chloro-2,3-dimethylquinazolin-4 (3H)-one and (2-fluoro-4-(trifluoromethyl)phenyl)boronic acid as starting materials to afford 6-chloro-8-(2-fluoro-4-(trifluoromethyl)phenyl)-2,3-dimethylquinazolin-4 (3H)-one as yellowish solid in 75.3% yield.

LCMS Condition K: Rt=2.52 min. m/z 371.2 [M+H]+.

### Step-4—Preparation of 6-(6-(1-cyclopropyl-1H-pyrazol-4-yl)-3,6-dihydro-2H-pyran-4-yl)-8-(2-fluoro-4-(trifluoromethyl)phenyl)-2,3-dimethylquinazolin-4 (3H)-one

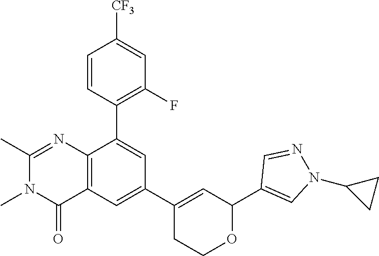 [SMILES: Cc1nc2c(-c3ccc(C(F)(F)F)cc3F)cc(C3=CC(c4cnn(C5CC5)c4)OCC3)cc2c(=O)n1C]

Procedure of Step-3, Example 14 was followed using 6-chloro-8-(2-fluoro-4-(trifluoromethyl)phenyl)-2,3-dimethylquinazolin-4 (3H)-one and 1-cyclopropyl-4-(4-(4,4,5,5-tetramethyl-1,3,2-dioxaborolan-2-yl)-5,6-dihydro-2H-pyran-2-yl)-1H-pyrazole (synthesized, step-2, Example 14) as starting materials to afford 6-(6-(1-cyclopropyl-1H-pyrazol-4-yl)-3,6-dihydro-2H-pyran-4-yl)-8-(2-fluoro-4-(trifluoromethyl)phenyl)-2,3-dimethylquinazolin-4 (3H)-one as brownish solid in 61.3% yield.

LCMS Condition K: Rt=2.49 min. m/z 525.2 [M+H]+.

### Step-5—Preparation of 6-(2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one

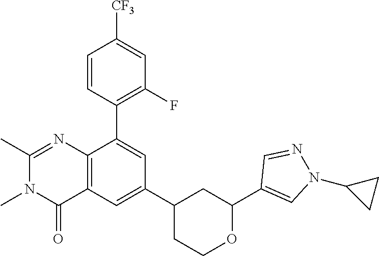 [SMILES: Cc1nc2c(-c3ccc(C(F)(F)F)cc3F)cc(C3CCOC(c4cnn(C5CC5)c4)C3)cc2c(=O)n1C]

Procedure of Step-2, Example 88A was followed using 6-(6-(1-cyclopropyl-1H-pyrazol-4-yl)-3,6-dihydro-2H-pyran-4-yl)-8-(2-fluoro-4-(trifluoromethyl)phenyl)-2,3-dimethylquinazolin-4 (3H)-one as starting material followed by reverse phase prep HPLC purification to afford 6-(2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one as yellow solid in 51.3% yield.

Prep-HPLC: Preparative HPLC was done on Waters auto purification instrument.

Column name: Xbridge C18 (250×19 mm, 5μ) operating at ambient temperature and flow rate of 16 mL/min. Mobile phase: A=20 mM Ammonium Bicarbonate in water, B=Acetonitrile:MEOH (50:50); Gradient Profile: Mobile phase initial composition of 60% A and 40% B, then 40% A and 60% B in 3 min, then to 10% A and 90% B in 20 min., then to 5% A and 95% B in 21 min., held this composition up to 22 min. for column washing, then returned to initial composition in 23 min. and held till 25 min.

LCMS Condition K: Rt=3.03 min. m/z 527.2 [M+H]+.

### Step-6—Preparation of 6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one [Example 118A] and 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one [Example 118B]

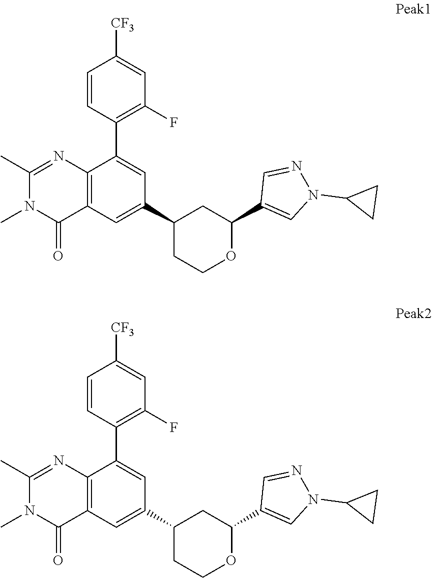 [SMILES: Cc1nc2c(-c3ccc(C(F)(F)F)cc3F)cc([C@@H]3CCO[C@H](c4cnn(C5CC5)c4)C3)cc2c(=O)n1C ; Cc1nc2c(-c3ccc(C(F)(F)F)cc3F)cc([C@H]3CCO[C@@H](c4cnn(C5CC5)c4)C3)cc2c(=O)n1C]

6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one [Example 118A, Peak1] and 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one [Example 118B, Peak2] were obtained by chiral separation of 6-(2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one by SFC. Absolute stereochemistry was assigned arbitrarily.

Chiral prep HPLC method details to separate the enantiomers:

Column: CHIRALCELOX-H (21.0 mm×250 mm), 5μ/Flow: 60 gm/min/Mobile Phase: 65% CO2+35% (100% MeOH)/UV: 220 nm/DILUENT: Methanol/Loading: 10.5 mg/11 min/Sample concentration: 35 mg/ml,

Example 118A, Peak1: 1H NMR (400 MHz, DMSO) δ 8.05 (d, J=1.5 Hz, 1H), 7.76-7.69 (m, 2H), 7.56-7.44 (m, 2H), 7.42-7.34 (m, 2H), 4.44 (d, J=10.4 Hz, 1H), 4.09-4.02 (m, 1H), 3.70-3.59 (m, 2H), 3.53 (s, 3H), 3.19-3.08 (m, 1H), 2.46 (s, 3H), 2.12-2.05 (m, 1H), 1.88-1.70 (m, 3H), 1.08-0.81 (m, 4H).

LCMS Condition K: Rt=3.03 min. m/z 527.2 [M+H]+.

Example 118B, Peak2: 1H NMR (400 MHz, DMSO) δ 8.05 (d, J=1.2 Hz, 1H), 7.76-7.69 (m, 2H), 7.56-7.44 (m, 2H), 7.42-7.35 (m, 2H), 4.43 (d, J=10.7 Hz, 1H), 4.12-3.99 (m, 1H), 3.70-3.59 (m, 2H), 3.53 (s, 3H), 3.19-3.08 (m, 1H), 2.46 (s, 3H), 2.12-2.04 (m, 1H), 1.88-1.67 (m, 3H), 1.04-0.83 (m, 4H).

LCMS Condition K: Rt=3.03 min. m/z 527.2 [M+H]+.

Analytical chiral HPLC by SFC: CHIRALCEL OX-H Mobile Phase: 0.3% IPAmine in MeOH: Flow Rate: 4.0 ml/min. Solubility: MeOH, Rt=4.22 min for Peak 1 and Rt=5.66 min for Peak 2.

### Example 119

### Example 119A—Peak1

### 8-(4-chloro-2-fluorophenyl)-6-((2S,4R)-2-(1-cyclopropyl-1H-pyrazol-4-yl)tetrahydro-2H-pyran-4-yl)-2,3-dimethylquinazolin-4 (3H)-one

### Example 119B—Peak2

### 8-(4-chloro-2-fluorophenyl)-6-((2R,4S)-2-(1-cyclopropyl-1H-pyrazol-4-yl)tetrahydro-2H-pyran-4-yl)-2,3-dimethylquinazolin-4 (3H)-one

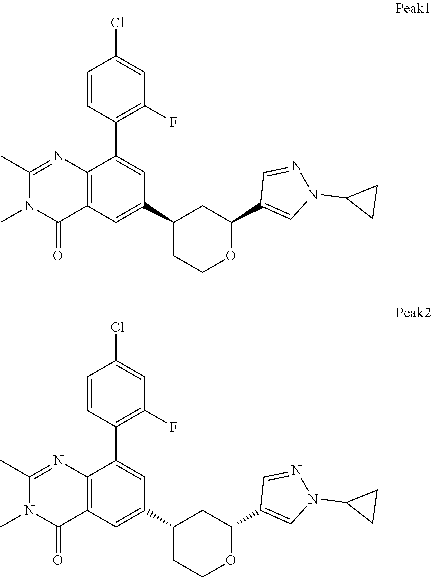 [SMILES: Cc1nc2c(-c3ccc(Cl)cc3F)cc([C@@H]3CCO[C@H](c4cnn(C5CC5)c4)C3)cc2c(=O)n1C ; Cc1nc2c(-c3ccc(Cl)cc3F)cc([C@H]3CCO[C@@H](c4cnn(C5CC5)c4)C3)cc2c(=O)n1C]

Synthesis of Example 119A—Peak1 and Example 119B—Peak2 involved 6 steps using 2-amino-3,5-dibromobenzoic acid as commercial starting material at 1st step and followed similar protocol as used for the synthesis and chiral separation of Example 118A—Peak1 and Example 118B—Peak2. Absolute stereochemistry was assigned arbitrarily.

Chiral separation of 8-(4-chloro-2-fluorophenyl)-6-(2-(1-cyclopropyl-1H-pyrazol-4-yl)tetrahydro-2H-pyran-4-yl)-2,3-dimethylquinazolin-4 (3H)-one by normal phase chiral prep HPLC afforded 8-(4-chloro-2-fluorophenyl)-6-((2S,4R)-2-(1-cyclopropyl-1H-pyrazol-4-yl)tetrahydro-2H-pyran-4-yl)-2,3-dimethylquinazolin-4 (3H)-one [arbitrarily chosen Example 119A—Peak1] as white solid eluted first and 8-(4-chloro-2-fluorophenyl)-6-((2R,4S)-2-(1-cyclopropyl-1H-pyrazol-4-yl)tetrahydro-2H-pyran-4-yl)-2,3-dimethylquinazolin-4 (3H)-one[arbitrarily chosen Example 119B—Peak2] as white solid eluted latter.

Example 119A, Peak1: 1H NMR (400 MHz, DMSO) δ 8.05 (d, J=1.5 Hz, 1H), 7.76-7.69 (m, 2H), 7.56-7.44 (m, 2H), 7.42-7.34 (m, 2H), 4.44 (d, J=10.4 Hz, 1H), 4.09-4.02 (m, 1H), 3.70-3.59 (m, 2H), 3.53 (s, 3H), 3.19-3.08 (m, 1H), 2.46 (s, 3H), 2.12-2.05 (m, 1H), 1.88-1.70 (m, 3H), 1.08-0.81 (m, 4H).

LCMS Condition K: Rt=2.99 min. m/z 493.4 [M+H]+.

Example 119B, Peak2: 1H NMR (400 MHz, DMSO) δ 8.05 (d, J=1.5 Hz, 1H), 7.76-7.69 (m, 2H), 7.56-7.44 (m, 2H), 7.42-7.34 (m, 2H), 4.44 (d, J=10.4 Hz, 1H), 4.09-4.02 (m, 1H), 3.70-3.59 (m, 2H), 3.53 (s, 3H), 3.19-3.08 (m, 1H), 2.46 (s, 3H), 2.12-2.05 (m, 1H), 1.88-1.70 (m, 3H), 1.08-0.81 (m, 4H).

LCMS Condition K: Rt=2.99 min. m/z 493.4 [M+H]+.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=11.29 min for Peak 1 and Rt=19.95 min for Peak 2.

### Example 120

### Example 120A—Peak1

### 6-((2S,4R)-2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one

### Example 120B—Peak2

### 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one

### Step-1—Preparation of 6-(6-(1-cyclopropyl-1H-pyrazol-4-yl)-3,6-dihydro-2H-pyran-4-yl)-8-(2,4-difluorophenyl)-2,3-dimethylpyrimido[5,4-d]pyrimidin-4 (3H)-one

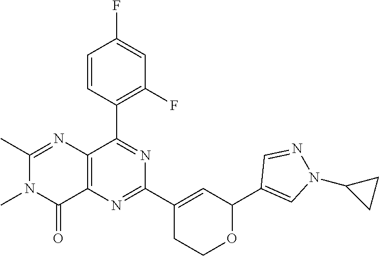 [SMILES: Cc1nc2c(-c3ccc(F)cc3F)nc(C3=CC(c4cnn(C5CC5)c4)OCC3)nc2c(=O)n1C]

Procedure of Step-3, Example 14 was followed using intermediate 3b and 1-cyclopropyl-4-(4-(4,4,5,5-tetramethyl-1,3,2-dioxaborolan-2-yl)-5,6-dihydro-2H-pyran-2-yl)-1H-pyrazole (step-2, Example 14) as starting materials to afford 6-(6-(1-cyclopropyl-1H-pyrazol-4-yl)-3,6-dihydro-2H-pyran-4-yl)-8-(2,4-difluorophenyl)-2,3-dimethylpyrimido[5,4-d]pyrimidin-4 (3H)-one as off-white solid in 42.1% yield.

LCMS Condition K: Rt=2.63 min. m/z 477.2 [M+H]+.

### Step-2—Preparation of 6-(2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one

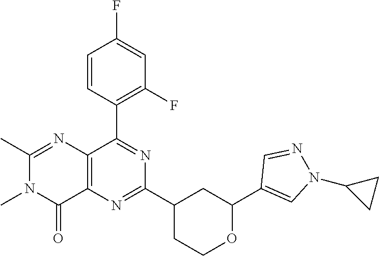 [SMILES: Cc1nc2c(-c3ccc(F)cc3F)nc(C3CCOC(c4cnn(C5CC5)c4)C3)nc2c(=O)n1C]

Procedure of Step-2, Example 88A was followed using 6-(6-(1-cyclopropyl-1H-pyrazol-4-yl)-3,6-dihydro-2H-pyran-4-yl)-8-(2,4-difluorophenyl)-2,3-dimethylpyrimido[5,4-d]pyrimidin-4 (3H)-one as starting material followed by reverse phase prep HPLC purification to afford 6-(2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one as off white solid in 39.3% yield.

Prep-HPLC: Preparative HPLC was done on Waters auto purification instrument. Column name: Xbridge C18 (250×19 mm, 5μ) operating at ambient temperature and flow rate of 16 mL/min. Mobile phase: A=20 mM Ammonium Bicarbonate in water, B=Acetonitrile:MEOH (50:50); Gradient Profile: Mobile phase initial composition of 60% A and 40% B, then 40% A and 60% B in 3 min, then to 10% A and 90% B in 20 min., then to 5% A and 95% B in 21 min., held this composition up to 22 min. for column washing, then returned to initial composition in 23 min. and held till 25 min

1H NMR (400 MHz, DMSO-d6) δ 7.79-7.69 (m, 2H), 7.50-7.39 (m, 1H), 7.36 (s, 1H), 7.33-7.24 (m, 1H), 4.49 (d, J=10.6 Hz, 1H), 4.12-4.04 (m, 1H), 3.74-3.60 (m, 2H), 3.56 (s, 3H), 3.49-3.36 (m, 1H), 2.52 (s, 3H), 2.21 (d, J=13.4 Hz, 1H), 2.05-1.77 (m, 3H), 1.04-0.82 (m, 4H).

LCMS Condition K: Rt=2.63 min. m/z 479.3 [M+H]+.

### Step-3—Preparation of 6-((2S,4R)-2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one [Example 120A] and 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one [Example 120B]

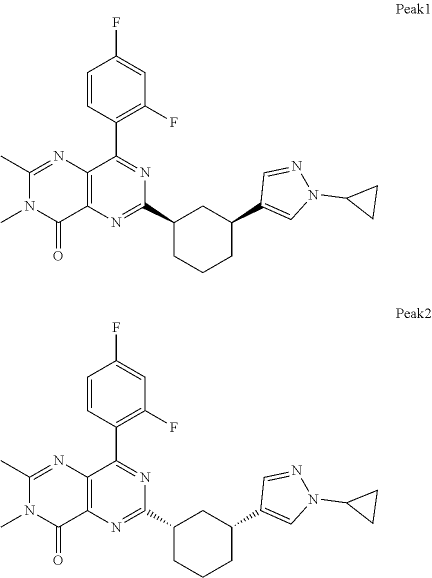 [SMILES: Cc1nc2c(-c3ccc(F)cc3F)nc([C@@H]3CCC[C@H](c4cnn(C5CC5)c4)C3)nc2c(=O)n1C ; Cc1nc2c(-c3ccc(F)cc3F)nc([C@H]3CCC[C@@H](c4cnn(C5CC5)c4)C3)nc2c(=O)n1C]

Chiral separation of 6-(2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one by normal phase chiral prep HPLC afforded 6-((2S,4R)-2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one [arbitrarily chosen Example 120A—Peak1] as off-white solid eluted first and 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one [arbitrarily chosen Example 120B—Peak2] as off-white solid eluted latter. Absolute stereochemistry was assigned arbitrarily.

Example 120A, Peak1: 1H NMR (400 MHz, DMSO-d6) δ 7.79-7.69 (m, 2H), 7.50-7.39 (m, 1H), 7.36 (s, 1H), 7.33-7.24 (m, 1H), 4.49 (d, J=10.6 Hz, 1H), 4.12-4.04 (m, 1H), 3.74-3.60 (m, 2H), 3.56 (s, 3H), 3.49-3.36 (m, 1H), 2.52 (s, 3H), 2.21 (d, J=13.4 Hz, 1H), 2.05-1.77 (m, 3H), 1.04-0.82 (m, 4H).

LCMS Condition K: Rt=2.63 min. m/z 479.3 [M+H]+.

Example 120B, Peak2: 1H NMR (400 MHz, DMSO-d6) δ 7.79-7.69 (m, 2H), 7.50-7.39 (m, 1H), 7.36 (s, 1H), 7.33-7.24 (m, 1H), 4.49 (d, J=10.6 Hz, 1H), 4.12-4.04 (m, 1H), 3.74-3.60 (m, 2H), 3.56 (s, 3H), 3.49-3.36 (m, 1H), 2.52 (s, 3H), 2.21 (d, J=13.4 Hz, 1H), 2.05-1.77 (m, 3H), 1.04-0.82 (m, 4H).

LCMS Condition K: Rt=2.63 min. m/z 479.3 [M+H]⁺.

Analytical chiral HPLS: Chiralpak IG (4.6×250 mm), 5μ Mobile Phase: Hexane/DCM/EtOH/IPAmine: 50/25/25/0.1 Flow Rate: 1.0 ml/min. Solubility: MeOH, Rt=9.77 min for Peak 1 and Rt=16.98 min for Peak 2.

### Example 121 to Example 124

Example 121 to Example 124 were synthesized using similar procedures as Example 4-10. Details of Example 121 to example 124 are captured in following table:

| | | | | |
|---|---|---|---|---|
| | | | Intermediate | |
| Compound | | | compound/SMs | LCMS |
| No. | Structure | Name | used | Conditions |

| | | | |
|---|---|---|---|
| Ex. 121 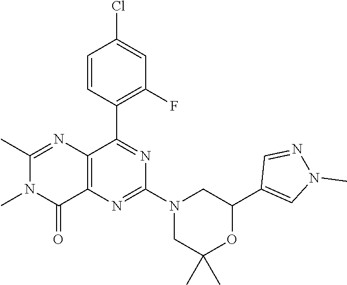 | 8-(2,4- difluorophenyl)- 6-(2,2-dimethyl- 6-(1-methyl-1H- pyrazol-4- yl)morpholino)- 2,3- dimethylpyrimido [5,4-d]pyrimidin- 4(3H)-one | 2,2-dimethyl-6-(1- methyl-1H-pyrazol- 4-yl)morpholine 6-chloro-8-(2,4- difluorophenyl)-2,3- dimethylpyrimido [5,4-d]pyrimidin- 4(3H)-one | Condition K: Rt = 2.75 min. m/z 482.4 [M + H]⁺. |
| Ex. 122 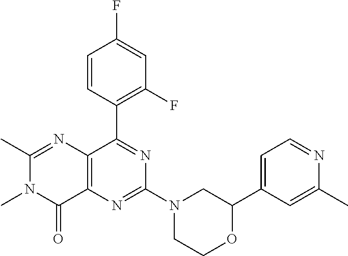 | 8-(2,4- difluorophenyl)- 2,3-dimethyl-6- (2-(2- methylpyridin-4- yl)morpholino) pyrimido[5,4- d]pyrimidin- 4(3H)-one | 2-(2-methylpyridin- 4-yl)morpholine 6-chloro-8-(2,4- difluorophenyl)-2,3- dimethylpyrido [5,4-d]pyrimidin- 4(3H)-one | Condition K: Rt = 2.33 min. m/z 465.3 [M + H]⁺. |
| Ex. 123 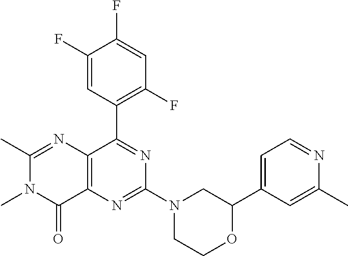 | 2,3-dimethyl-6- (2-(2- methylpyridin-4- yl)morpholino)- 8-(2,4,5- trifluorophenyl) pyrimido[5,4- d]pyrimidin- 4(3H)-one | 2-(2-methylpyridin- 4-yl)morpholine 6-chloro-2,3- dimethyl-8-(2,4,5- trifluorophenyl) pyrimido[5,4- d]pyrimidin-4(3H)- one | Condition K: Rt = 2.35 min. m/z 483.3 [M + H]⁺. |
| Ex. 124 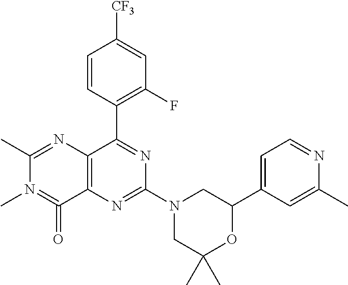 | 6-(2,2-dimethyl- 6-(2- methylpyridin-4- yl)morpholino)- 8-(2-fluoro-4- (trifluoromethyl) phenyl)-2,3- dimethylpyrimido [5,4-d]pyrimidin- 4(3H)-one | 2,2-dimethyl-6-(2- methylpyridin-4- yl)morpholine 6-chloro-8-(2- fluoro-4- (trifluoromethyl) phenyl)-2,3- dimethylpyrimido [5,4-d]pyrimidin- 4(3H)-one | Condition K: Rt = 2.35 min. m/z 543.3 [M + H]⁺. |

Chiral separation details of Example 121A-121B to 124A-124B are captured in following table:

| | | | | |
|---|---|---|---|---|
| | | | | Chiral |
| Compound | | | Starting | separation |
| No. | Structure | Name | material | specification |

| | | | |
|---|---|---|---|
| Ex. 121A (Peak 1) 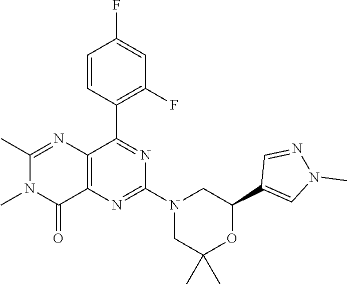 | (R)-8-(2,4- difluorophenyl)-6- (2,2-dimethyl-6-(1- methyl-1H-pyrazol-4- yl)morpholino)-2,3- dimethylpyrimido[5,4- d]pyrimidin-4(3H)- one | 8-(2,4-difluorophenyl)-6-(2,2- dimethyl-6-(1-methyl-1H- pyrazol-4-yl)morpholino)- 2,3-dimethylpyrimido[5,4- d]pyrimidin-4(3H)-one | Similar specifications as mentioned in step-6 of example 1. Absolute stereochemistry unknown |
| Ex. 121B (Peak 2) 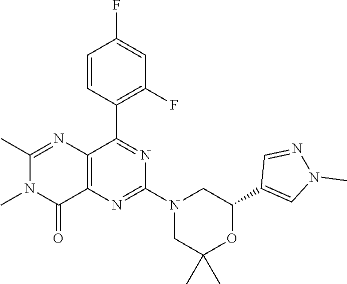 | (S)-8-(2,4- difluorophenyl)-6- (2,2-dimethyl-6-(1- methyl-1H-pyrazol-4- yl)morpholino)-2,3- dimethylpyrimido [5,4-d]pyrimidin- 4(3H)-one | | Absolute stereochemistry unknown |
| Ex. 122A (Peak 1) 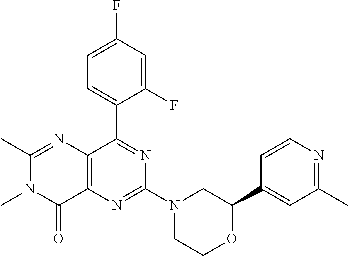 | (R)-8-(2,4- difluorophenyl)-2,3- dimethyl-6-(2-(2- methylpyridin-4- yl)morpholino) pyrimido[5,4-d] pyrimidin-4(3H)-one | 8-(2,4-difluorophenyl)-2,3- dimethyl-6-(2-(2- methylpyridin-4-yl) morpholino)pyrimido [5,4-d]pyrimidin-4(3H)-one | Similar specifications as mentioned in step-6 of example 1. Absolute stereochemistry unknown |
| Ex. 122B (Peak 2) 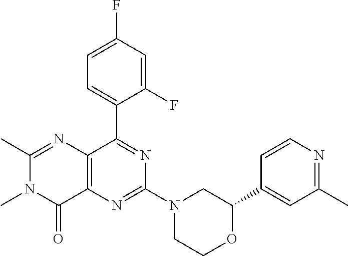 | (S)-8-(2,4- difluorophenyl)-2,3- dimethyl-6-(2-(2- methylpyridin-4- yl)morpholino) pyrimido[5,4-d] pyrimidin-4(3H)-one | | Absolute stereochemistry unknown |
| Ex. 123A (Peak 1) 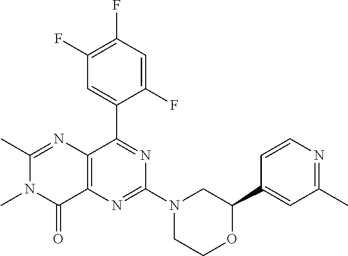 | (R)-2,3-dimethyl-8- (2-(2-methylpyridin- 4-yl)morpholino)-8- (2,4,5- trifluorophenyl) pyrimido[5,4-d] pyrimidin-4(3H)-one | 2,3-dimethyl-6- (2-(2-methylpyridin-4- yl)morpholino)-8-(2,4,5- trifluorophenyl) pyrimido[5,4-d]pyrimidin- 4(3H)-one | Similar specifications as mentioned in step-6 of example-1. Absolute stereochemistry unknown |
| Ex. 123B (Peak 2) 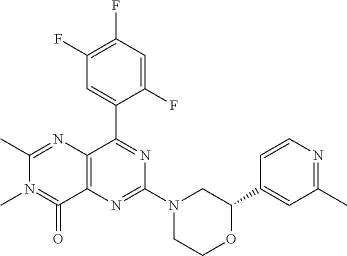 | (S)-2,3-dimethyl-6- (2-(2-methylpyridin- 4-yl)morpholino)-8- (2,4,5-trifluorophenyl) pyrimido[5,4-d] pyrimidin-4(3H)-one | | Absolute stereochemistry unknown |
| Ex. 124A (Peak 1) 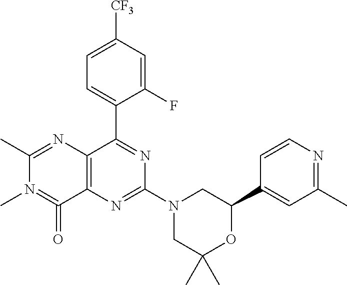 | (R)-6-(2,2-dimethyl- 6-(2-methylpyridin-4- yl)morpholino)-8-(2- fluoro-4- (trifluoromethyl) phenyl)-2,3- dimethylpyrimido [5,4-d]pyrimidin- 4(3H)-one | 6-(2,2-dimethyl-6-(2- methylpyridin-4- yl)morpholino)-8-(2- fluoro-4-(trifluoromethyl) phenyl)-2,3-dimethyl- pyrimido[5,4-d]pyrimidin- 4(3H)-one | Agilent 1200 series instrument. Column name: CHIRALART CELLULOSE SC (250 × 20 mm) 5 u. Operating at ambient temperature and flow rate is 18.0 mL/min. Mobile phase was mixture of 75% Hexane, 12.5% DCM and 12.5% ETOH, held this isocratic mixture run upto 25 min with wavelength of 296 nm. Absolute stereochemistry unknown |
| Ex. 124B (Peak 2) 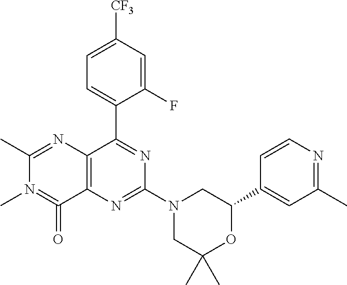 | (S)-6-(2,2-dimethyl- 6-(2-methylpyridin-4- yl)morpholino)-8-(2- fluoro-4- (trifluoromethyl) phenyl)-2,3- dimethylpyrimido [5,4-d]pyrimidin- 4(3H)-one | | Absolute stereochemistry unknown |

Following table describes analytical data analysis and yield information of examples 121A-121B to examples 124A-124B:

| Compound | | | Analytical chiral | | Yield |
|---|---|---|---|---|---|
| No. | HNMR | LCMS | HPLC | Description | (%) |
| Ex. 121A | 1H NMR (400 MHz, DMSO) δ | Condition | CHIRALPAK IG | Yellow | 35.7 |
| (Peak 1) | 7.75-7.64 (m, 2H), 7.46- | K: Rt = | (250 × 4.6) mm, 5 μ | solid | |
| | 7.36 (m, 2H), 7.25 (t, J = 7.6 | 2.75 min. | M.P.-Hexane/DCM/ | | |
| | Hz, 1H), 4.85-4.65 (m, 2H), | m/z 482.4 | EtOH/IPAmine- | | |
| | 4.53 (d, J = 12.9 Hz, 1H), | [M + H]⁺. | 50/25/25/0.1 Flow | | |
| | 3.80 (s, 3H), 3.51 (s, 3H), | | Rate-1.0 ml/min | | |
| | 3.03-2.88 (m, 2H), 2.43 (s, | | Solubility: MeOH | | |
| | 3H), 1.23 (s, 6H). | | Rt = 6.55 min for | | |
| Ex. 121B | 1H NMR (400 MHz, DMSO) δ | Condition | Peak 1 and Rt = | Yellow | 35.3 |
| (Peak 2) | 7.75-7.64 (m, 2H), 7.46- | K: Rt = | 11.48 min for Peak 2. | solid | |
| | 7.36 (m, 2H), 7.29-7.20 (m, | 2.75 min. | | | |
| | 1H), 4.85-4.66 (m, 2H), | m/z 482.4 | | | |
| | 4.53 (d, J = 12.9 Hz, 1H), | [M + H]⁺. | | | |
| | 3.80 (s, 3H), 3.50 (s, 3H), | | | | |
| | 3.03-2.88 (m, 2H), 2.43 (s, | | | | |
| | 3H), 1.23 (s, 6H). | | | | |
| Ex. 122A | 1H NMR (400 MHz, Methanol-d4) | Condition | CHIRALPAK IG | Yellow | 38.3 |
| (Peak 1) | δ 8.40 (d, J = 5.2 Hz, 1H), | K: Rt = | (250 × 4.6) mm, 5 μ | solid | |
| | 7.76-7.65 (m, 1H), 7.47 (s, | 2.33 min. | M.P.-Hexane/ | | |
| | 1H), 7.39 (d, J = 5.2 Hz, | m/z 465.3 | DCM/EtOH/ | | |
| | 1H), 7.19-6.96 (m, 2H), 5.12- | [M + H]⁺. | IPAmine- | | |
| | 5.07 (m, 1H), 4.84-4.76 (m, | | 50/25/25/0.1 Flow | | |
| | 1H), 4.61 (d, J = 9.0 Hz, | | Rate-1.0 ml/min | | |
| | 1H), 4.17 (d, J = 11.0 Hz, 1H), | | Solubility: MeOH | | |
| | 3.80 (t, J = 10.6 Hz, 1H), | | Rt = 6.63 min for | | |
| | 3.64 (s, 3H), 3.27-3.19 (m, | | Peak 1 and Rt = | | |
| | 1H), 3.00-2.89 (m, 1H), 2.54 | | 5.59 min for Peak 2. | | |
| | (d, J = 13.0 Hz, 6H). | | | | |
| Ex. 122B | 1H NMR (400 MHz, | Condition | | Yellow | 38.0 |
| (Peak 2) | Methanol-d4) δ 8.40 (d, J = | K: Rt = | | solid | |
| | 5.1 Hz, 1H), 7.76-7.65 (m, | 2.33 min. | | | |
| | 1H), 7.47 (s, 1H), 7.39 (d, J = | m/z 465.3 | | | |
| | 4.6 Hz, 1H), 7.15-7.05 (m, | [M + H]⁺. | | | |
| | 2H), 5.13-5.07 (m, 1H), | | | | |
| | 4.84-4.76 (m, 1H), 4.61 (d, | | | | |
| | J = 8.5 Hz, 1H), 4.21-4.13 | | | | |
| | (m, 1H), 3.86-3.76 (m, 1H), | | | | |
| | 3.64 (s, 3H), 3.27-3.19 (m, | | | | |
| | 1H), 3.00-2.89 (m, 1H), | | | | |
| | 2.54 (d, J = 12.9 Hz, 6H). | | | | |
| Ex. 123A | 1H NMR (400 MHz, | Condition | CHIRALPAK IG | Yellow | 36.4 |
| (Peak 1) | Methanol-d4) δ 8.40 (d, J = | K: Rt = | (250 × 4.6) mm, 5 μ | solid | |
| | 4.7 Hz, 1H), 7.69-7.62 (m, | 2.35 min. | M.P.-Hexane/ | | |
| | 1H), 7.46 (s, 1H), 7.42-7.27 | m/z 483.3 | DCM/EtOH/ | | |
| | (m, 2H), 5.12-5.07 (m, 1H), | [M + H]⁺. | IPAmine- | | |
| | 4.83-4.75 (m, 1H), 4.65- | | 50/25/25/0.1 Flow | | |
| | 4.58 (m, 1H), 4.30-4.05 (m, | | Rate-1.0 ml/min | | |
| | 1H), 3.80 (t, J = 10.1 Hz, 1H), | | Solubility: MeOH | | |
| | 3.64 (s, 3H), 3.29-3.17 (m, | | Rt = 6.35 min for | | |
| | 1H), 2.95 (t, J = 10.7 Hz, 1H), | | Peak 1 and Rt = | | |
| | 2.54 (d, J = 9.3 Hz, 6H). | | 9.07 min for Peak | | |
| Ex. 123B | 1H NMR (400 MHz, | Condition | 2. | Yellow | 36.0 |
| (Peak 2) | Methanol-d4) δ 8.40 (d, J = | K: Rt = | | solid | |
| | 4.9 Hz, 1H), 7.73-7.59 (m, | 2.35 min. | | | |
| | 1H), 7.46 (s, 1H), 7.43-7.24 | m/z 483.3 | | | |
| | (m, 2H), 5.23-4.97 (m, 1H), | [M + H]⁺. | | | |
| | 4.83-4.75 (m, 1H), 4.61 (d, | | | | |
| | J = 10.3 Hz, 1H), 4.22-4.13 | | | | |
| | (m, 1H), 3.80 (t, J = 11.2 Hz, | | | | |
| | 1H), 3.64 (s, 3H), 3.30-3.18 | | | | |
| | (m, 1H), 2.95 (t, J = 11.0 Hz, | | | | |
| | 1H), 2.54 (d, J = 9.3 Hz, 6H). | | | | |
| Ex. 124A | 1H NMR (400 MHz, | Condition | Column Name: | Yellow | 34.4 |
| (Peak 1) | Methanol-d4) δ 8.38 (d, J = | K: Rt = | Chiralpak IC (4.6 × | solid | |
| | 5.2 Hz, 1H), 7.83 (t, J = 6.6 | 2.65 min. | 250 mm), 5 μ | | |
| | Hz, 1H), 7.65 (d, J = 8.2 Hz, | m/z 543.4 | ARD/K/7788 | | |
| | 1H), 7.61 (d, J = 9.6 Hz, 1H), | [M + H]⁺. | Mobile Phase: | | |
| | 7.53-7.33 (m, 2H), 5.35- | | Hexane/DCM/EtOH/ | | |
| | 5.06 (m, 1H), 4.99-4.88 (m, | | IPAmine: | | |
| | 2H), 3.65 (s, 3H), 2.99 (d, J = | | 50/25/25/0.1 | | |
| | 13.1 Hz, 1H), 2.80 (dd, J = | | Flow Rate: 1.0 | | |
| | 10.9, 13.2 Hz, 1H), 2.53 (d, | | ml/min Solubility: | | |
| | J = 15.5 Hz, 6H), 1.39 (s, 3H), | | MeOH Rt = 5.28 | | |
| | 1.32 (s, 3H). | | min for Peak 1 and | | |
| Ex. 124B | 1H NMR (400 MHz, | Condition | Rt = 6.71 min for | Yellow | 34.3 |
| (Peak 2) | Methanol-d4) δ 8.38 (d, J = | K: Rt = | Peak 2. | solid | |
| | 5.0 Hz, 1H), 7.83 (t, J = 7.5 | 2.65 min. | | | |
| | Hz, 1H), 7.65 (d, J = 7.8 Hz, | m/z 543.4 | | | |
| | 1H), 7.61 (d, J = 9.8 Hz, 1H), | [M + H]⁺. | | | |
| | 7.46 (s, 1H), 7.39 (s, 1H), | | | | |
| | 5.36-5.03 (m, 1H), 4.98- | | | | |
| | 4.90 (m, 2H), 3.65 (s, 3H), | | | | |
| | 2.99 (d, J = 13.2 Hz, 1H), | | | | |
| | 2.85-2.74 (m, 1H), 2.53 (d, | | | | |
| | J = 15.4 Hz, 6H), 1.39 (s, | | | | |
| | 3H), 1.32 (s, 3H). | | | | |

### Example 125 to Example 141

Example 125 to Example 141 were synthesized using similar procedures as Example 112.

| | | | | |
|---|---|---|---|---|
| | | | Intermediate | |
| Compound | | | compounds/SMs | |
| No. | Structure | Name | used | LCMS Data |

| | | | |
|---|---|---|---|
| Ex. 125 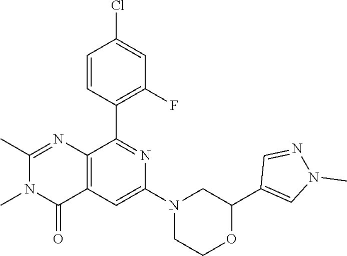 | 8-(4-chloro-2- fluorophenyl)- 2,3-dimethyl-6- (2-(1-methyl- 1H-pyrazol-4- yl)morpholino) pyrido[3,4- d]pyrimidin- 4(3H)-one | 2-(1-methyl-1H- pyrazol-4- yl)morpholine 6-chloro-8-(4-chloro- 2-fluorophenyl)-2,3- dimethylpyrido[3,4- d]pyrimidin-4(3H)- one | Condition K: Rt = 2.91 min. m/z 469.3 [M + H]⁺. |
| Ex. 126 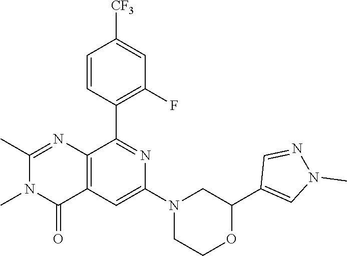 | 8-(2-fluoro-4- (trifluoromethyl) phenyl)-2,3- dimethyl-6-(2- (1-methyl-1H- pyrazol-4- yl)morpholino) pyrido[3,4- d]pyrimidin- 4(3H)-one | 2-(1-methyl-1H- pyrazol-4- yl)morpholine 6-chloro-8-(2-fluoro- 4- (trifluoromethyl) phenyl)-2,3- dimethylpyrido[3,4- d]pyrimidin-4(3H)- one | Condition K: Rt = 3.27 min. m/z 503.2 [M + H]⁺. |
| Ex. 127 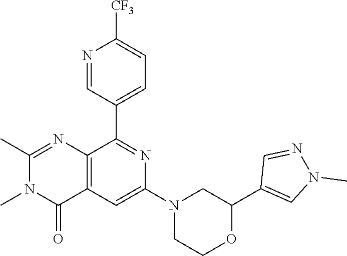 | 2,3-dimethyl-6- (2-(1-methyl- 1H-pyrazol-4- yl)morpholino)- 8-(6- (trifluoromethyl) pyridin-3- yl)pyrido[3,4- d]pyrimidin- 4(3H)-one | 2-(1-methyl-1H- pyrazol-4- yl)morpholine 6-chloro-2,3- dimethyl-8-(6- (trifluoromethyl) pyridin-3-yl)pyrido [3,4-d]pyrimidin- 4(3H)-one | Condition K: Rt = 3.14 min. m/z 486.2 [M + H]⁺. |

Chiral separation details of Example 125A-1251B to 141A-141B are captured in following table:

| | | | | |
|---|---|---|---|---|
| | | | | Chiral |
| Compound | | | Starting | separation |
| No. | Structure | Name | material | specification |

| | | | |
|---|---|---|---|
| Ex. 125A (Peak 1) 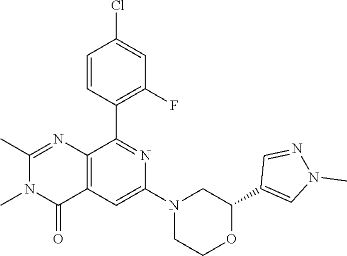 | (S)-8-(4-chloro- 2-fluorophenyl)- 2,3-dimethyl-6- (2-(1-methyl-1H- pyrazol-4- yl)morpholino) pyrido[3,4-d] pyrimidin-4(3H)- one | 8-(4-chloro-2- fluorophenyl)- 2,3-dimethyl-6- (2-(1-methyl- 1H-pyrazol-4- yl)morpholino) pyrido[3,4- d]pyrimidin- 4(3H)-one | Similar specifications as mentioned in step-6 of example 1. Absolute stereochemistry unknonw |
| Ex. 125B (Peak 2) 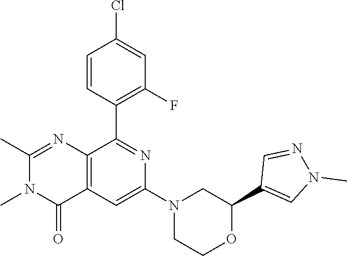 | (R)-8-(4-chloro- 2-fluorophenyl)- 2,3-dimethyl-6- (2-(1-methyl-1H- pyrazol-4- yl)morpholino) pyrido[3,4- d]pyrimidin- 4(3H)-one | | Absolute stereochemistry unknown |
| Ex. 126A (Peak 1) 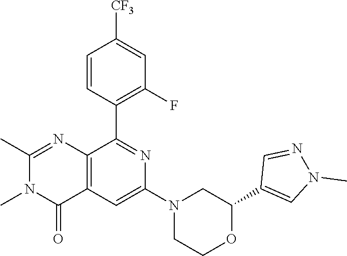 | (S)-8-(2-fluoro- 4- (trifluoromethyl) phenyl)-2,3- dimethyl-6-(2-(1- methyl-1H- pyrazol-4- yl)morpholino) pyrido[3,4-d] pyrimidin- 4(3H)-one | 8-(2-fluoro-4- (trifluoromethyl) phenyl)-2,3- dimethyl-6-(2- (1-methyl-1H- pyrazol-4- yl)morpholino) pyrido[3,4- d]pyrimidin- 4(3H)-one | Similar specifications as mentioned in step-6 of example-1. Absolute stereochemistry unknown |
| Ex. 126B (Peak 2) 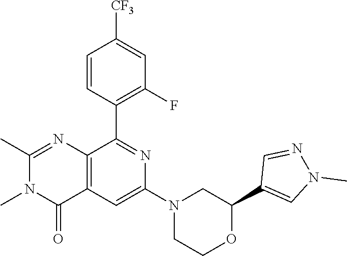 | (R)-8-(2-fluoro- 4- (trifluoromethyl) phenyl)-2,3- dimethyl-6-(2-(1- methyl-1H- pyrazol-4- yl)morpholino) pyrido[3,4- d]pyrimidin- 4(3H)-one | | Absolute stereochemistry unknown |
| Ex. 127A (Peak 1) 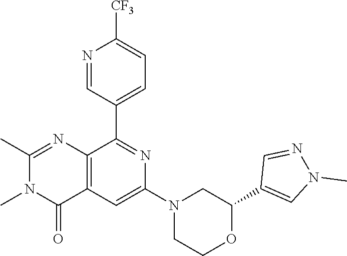 | (S)-2,3- dimethyl-6-(2-(1- methyl-1H- pyrazol-4- yl)morpholino)- 8-(6- (trifluoromethyl) pyridin-3- yl)pyrido[3,4- d]pyrimidin- 4(3H)-one | 2,3-dimethyl-6- (2-(1-methyl- 1H-pyrazol-4- yl)morpholino)- 8-(6- (trifluoromethyl) pyridin-3- yl)pyrido[3,4- d]pyrimidin- 4(3H)-one | Agilent 1200 series instrument. Column name: CHIRALART CELLULOSE SC (250 × 20 mm) 5 u. Operating at ambient temperature and flow rate is 18.0 mL/min. Mobile phase was mixture of 75% Hexane, 12.5% DCM and 12.5% ETOH, held this isocratic mixture run upto 25 min with wavelength of 296 nm. Absolute stereochemistry unknown |
| Ex. 127B (Peak 2) 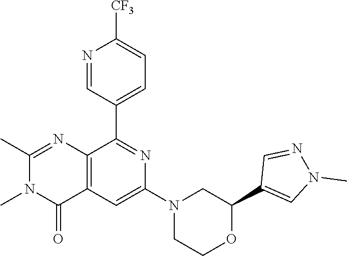 | (R)-2,3- dimethyl-6-(2-(1- methyl-1H- pyrazol-4- yl)morpholino)- 8-(6- (trifluoromethyl) pyridin-3-yl) pyrido[3,4-d] pyrimidin- 4(3H)-one | | Absolute stereochemistry unknown |
| Ex. 128A (Peak 1) 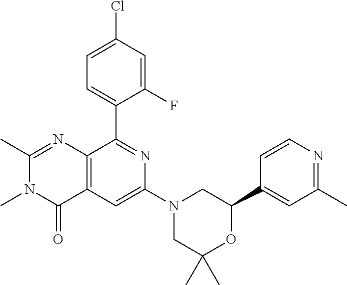 | (R)-8-(4-chloro- 2-fluorophenyl)- 2,3-dimethyl-6- (5-(2- methylpyridin-4- yl)-4-oxa-7- azaspiro[2.5] octan-7-yl) pyrido[3,4-d] pyrimidin- 4(3H)-one | 8-(4-chloro-2- fluorophenyl)- 2,3-dimethyl-6- (5-(2- methylpyridin- 4-yl)-4-oxa-7- azaspiro[2.5] octan-7- yl)pyrido[3,4- d]pyrimidin- 4(3H)-one | Similar specifications as mentioned in step-6 of example 1 Peak 1 Absolute stereochemistry unknown |
| Ex. 128B (Peak 2) 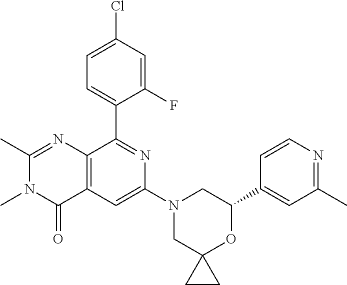 | (S)-8-(4-chloro- 2-fluorophenyl)- 2,3-dimethyl-6- (5-(2- methylpyridin-4- yl)-4-oxa-7- azaspiro[2.5] octan-7- yl)pyrido[3,4- d]pyrimidin- 4(3H)-one | | Peak 2 Absolute stereochemistry unknown |
| Ex. 129A (Peak 1) 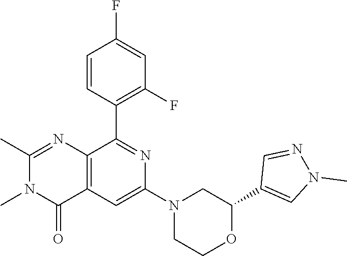 | (S)-8-(2,4- difluorophenyl)- 2,3-dimethyl-6- (2-(1-methyl-1H- pyrazol-4- yl)morpholino) pyrido[3,4- d]pyrimidin- 4(3H)-one | 8-(2,4- difluorophenyl)- 2,3-dimethyl- 6-(2-(1-methyl- 1H-pyrazol-4- yl)morpholino) pyrido[3,4- d]pyrimidin- 4(3H)-one | Similar specifications as mentioned in step-6 of example 1 Peak 1 Absolute stereochemistry unknown |
| Ex. 129B (Peak 2) 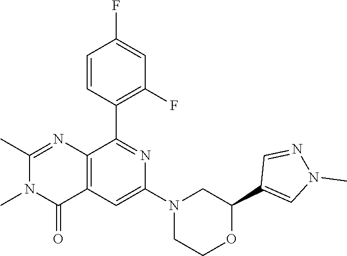 | (R)-8-(2,4- difluorophenyl)- 2,3-dimethyl-6- (2-(1-methyl-1H- pyrazol-4- yl)morpholino) pyrido[3,4-d] pyrimidin- 4(3H)-one | | Peak 2 Absolute stereochemistry unknown |
| Ex. 130A (Peak 1) 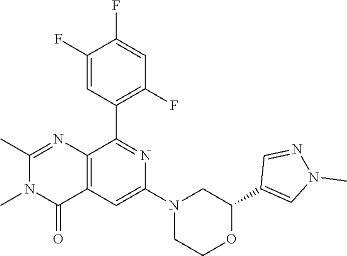 | (S)-2,3- dimethyl-6-(2-(1- methyl-1H- pyrazol-4- yl)morpholino)- 8-(2,4,5- trifluorophenyl) pyrido[3,4- d]pyrimidin- 4(3H)-one | 2,3-dimethyl-6- (2-(1-methyl- 1H-pyrazol-4- yl)morpholino)- 8-(2,4,5- trifluorophenyl) pyrido[3,4- d]pyrimidin- 4(3H)-one | Similar specifications as mentioned in step-6 of example 1 Peak 1 Absolute stereochemistry unknown |
| Ex. 130B (Peak 2) 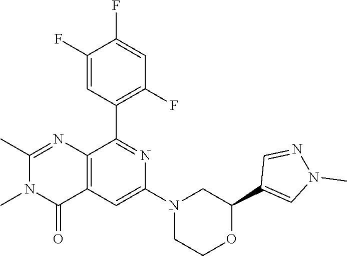 | (R)-2,3- dimethyl-6-(2-(1- methyl-1H- pyrazol-4- yl)morpholino)- 8-(2,4,5- trifluorophenyl) pyrido[3,4-d] pyrimidin- 4(3H)-one | | Peak 2 Absolute stereochemistry unknown |
| Ex. 131A (Peak 1) 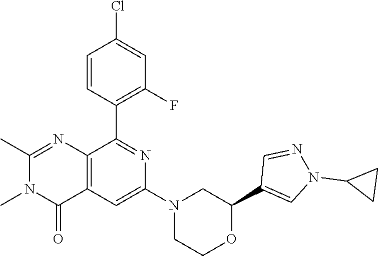 [SMILES: Cc1nc2c(-c3ccc(Cl)cc3F)nc(N3CCO[C@H](c4cnn(C5CC5)c4)C3)cc2c(=O)n1C] | (R)-8-(4-chloro- 2-fluorophenyl) 6-(2-(1- cyclopropyl-1H- pyrazol-4- yl)morpholino)- 2,3- dimethylpyrido [3,4-d]pyrimidin- 4(3H)-one | 8-(4-chloro-2- fluorophenyl)- 6-(2-(1- cyclopropyl- 1H-pyrazol-4- yl)morpholino)- 2,3- dimethylpyrido [3,4-d] pyrimidin-4(3H)- one | Similar specifications as mentioned in step-6 of example 1 Peak 1 Absolute stereochemistry unknown |
| Ex. 131B (Peak 2) 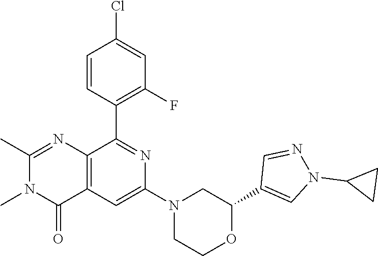 [SMILES: Cc1nc2c(-c3ccc(Cl)cc3F)nc(N3CCO[C@@H](c4cnn(C5CC5)c4)C3)cc2c(=O)n1C] | (S)-8-(4-chloro- 2-fluorophenyl)- 6-(2-(1- cyclopropyl-1H- pyrazol-4- yl)morpholino)- 2,3- dimethylpyrido [3,4-d]pyrimidin- 4(3H)-one | | Peak 2 Absolute stereochemistry unknown |
| Ex. 132A (Peak 1) 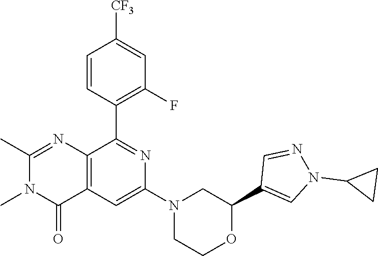 [SMILES: Cc1nc2c(-c3ccc(C(F)(F)F)cc3F)nc(N3CCO[C@H](c4cnn(C5CC5)c4)C3)cc2c(=O)n1C] | (R)-6-(2-(1- cyclopropyl-1H- pyrazol-4- yl)morpholino)- 8-(2-fluoro-4- (trifluoromethyl) phenyl)-2,3- dimethylpyrido [3,4-d]pyrimidin- 4(3H)-one | 6-(2-(1- cyclopropyl- 1H-pyrazol-4- yl)morpholino)- 8-(2-fluoro-4- (trifluoromethyl) phenyl)-2,3- dimethylpyrido [3,4-d] pyrimidin- 4(3H)-one | Similar specifications as mentioned in step-6 of example 1 Peak 1 Absolute stereochemistry unknown |
| Ex. 132B (Peak 2) 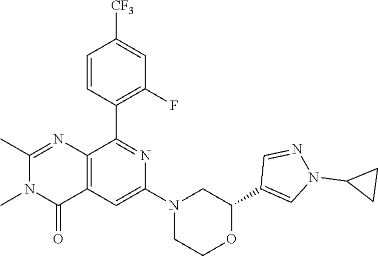 [SMILES: Cc1nc2c(-c3ccc(C(F)(F)F)cc3F)nc(N3CCO[C@@H](c4cnn(C5CC5)c4)C3)cc2c(=O)n1C] | (S)-6-(2-(1- cyclopropyl-1H- pyrazol-4- yl)morpholino)- 8-(2-fluoro-4- (trifluoromethyl) phenyl)-2,3- dimethylpyrido [3,4-d]pyrimidin- 4(3H)-one | | Peak 2 Absolute stereochemistry unknown |
| Ex. 133A (Peak 1) 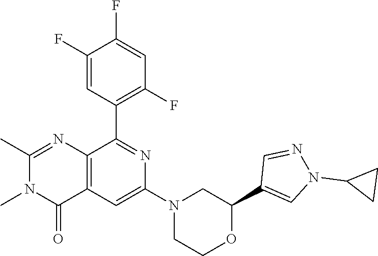 [SMILES: Cc1nc2c(-c3cc(F)c(F)cc3F)nc(N3CCO[C@H](c4cnn(C5CC5)c4)C3)cc2c(=O)n1C] | (R)-6-(2-(1- cyclopropyl-1H- pyrazol-4- yl)morpholino)- 2,3-dimethyl-8- (2,4,5- trifluorophenyl) pyrido[3,4-d] pyrimidin- 4(3H)-one | 6-(2-(1- cyclopropyl- 1H-pyrazol-4- yl)morpholino)- 2,3-dimethyl-8- (2,4,5- trifluorophenyl) pyrido[3,4-d] pyrimidin- 4(3H)-one | Similar specifications as mentioned in step-6 of example 1 Peak 1 Absolute stereochemistry unknown |
| Ex. 133B (Peak 2) 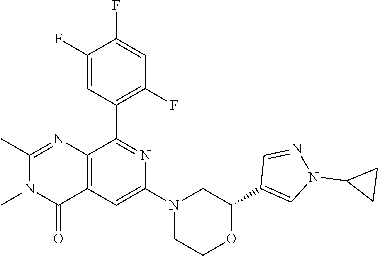 [SMILES: Cc1nc2c(-c3cc(F)c(F)cc3F)nc(N3CCO[C@@H](c4cnn(C5CC5)c4)C3)cc2c(=O)n1C] | (S)-6-(2-(1- cyclopropyl-1H- pyrazol-4- yl)morpholino)- 2,3-dimethyl-8- (2,4,5- trifluorophenyl) pyrido[3,4- d]pyrimidin- 4(3H)-one | | Peak 2 Absolute stereochemistry unknown |
| Ex. 134A (Peak 1) 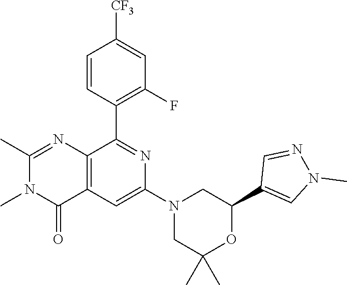 | (R)-6-(2,2- dimethyl-6-(1- methyl-1H- pyrazol-4- yl)morpholino)- 8-(2-fluoro-4- (trifluoromethyl) phenyl)-2,3- dimethylpyrido [3,4-d]pyrimidin- 4(3H)-one | 6-(2,2- dimethyl-6-(1- methyl-1H- pyrazol-4- yl)morpholino)- 8-(2-fluoro-4- (trifluoromethyl) phenyl)-2,3- dimethylpyrido [3,4- d]pyrimidin- 4(3H)-one | Similar specifications as mentioned in step-6 of example 1 Peak 1 Absolute stereochemistry unknown |
| Ex. 134B (Peak 2) 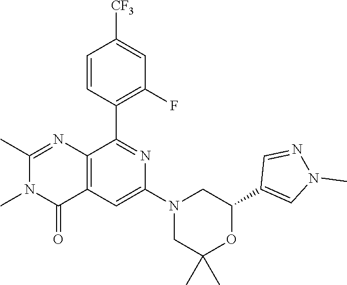 | (S)-6-(2,2- dimethyl-6-(1- methyl-1H- pyrazol-4- yl)morpholino)- 8-(2-fluoro-4- (trifluoromethyl) phenyl)-2,3- dimethylpyrido [3,4-d]pyrimidin- 4(3H)-one | | Peak 2 Absolute stereochemistry unknown |
| Ex. 135A (Peak 1) 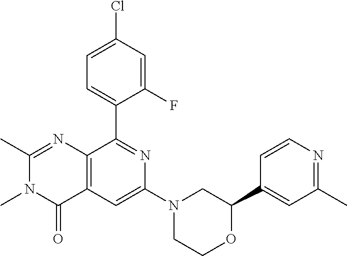 | (R)-8-(4-chloro- 2-fluorophenyl)- 2,3-dimethyl-6- (2-(2- methylpyridin-4- yl)morpholino) pyrido[3,4- d]pyrimidin- 4(3H)-one | 8-(4-chloro-2- fluorophenyl)- 2,3-dimethyl-6- (2-(2- methylpyridin- 4-yl) morpholino) pyrido[3,4-d] pyrimidin-4(3H)- one | Similar specifications as mentioned in step-6 of example 1 Peak 1 Absolute stereochemistry unknown |
| Ex. 135B (Peak 2) 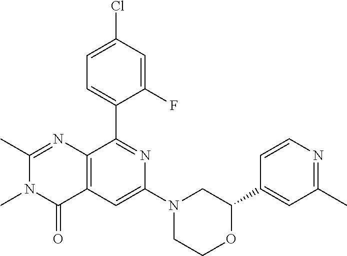 | (S)-8-(4-chloro- 2-fluorophenyl)- 2,3-dimethyl-6- (2-(2- methylpyridin-4- yl)morpholino) pyrido[3,4-d] pyrimidin- 4(3H)-one | | Peak 2 Absolute stereochemistry unknown |
| Ex. 136A (Peak 1) 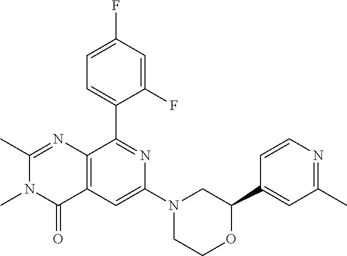 | (R)-8-(2,4- difluorophenyl)- 2,3-dimethyl-6- (2-(2- methylpyridin-4- yl)morpholino) pyrido[3,4- d]pyrimidin- 4(3H)-one | 8-(2,4- difluorophenyl)- 2,3-dimethyl- 6-(2-(2- methylpyridin-4- yl)morpholino) pyrido[3,4- d]pyrimidin- 4(3H)-one | Similar specifications as mentioned in step-6 of example 1 Peak 1 Absolute stereochemistry unknown |
| Ex. 136B (Peak 2) 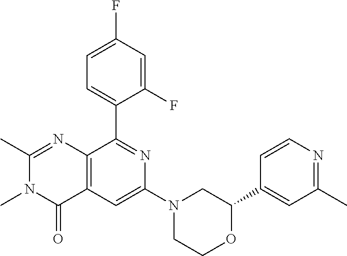 | (S)-8-(2,4- difluorophenyl)- 2,3-dimethyl-6- (2-(2- methylpyridin-4- yl)morpholino) pyrido[3,4-d] pyrimidin- 4(3H)-one | | Peak 2 Absolute stereochemistry unknown |
| Ex. 137A (Peak 1) 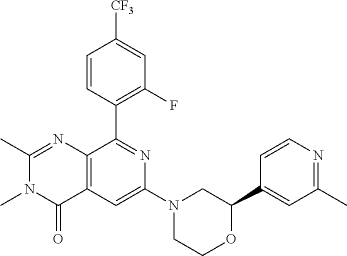 | (R)-8-(2-fluoro- 4- (trifluoromethyl) phenyl)-2,3- dimethyl-6-(2-(2- methylpyridin-4- yl)morpholino) pyrido[3,4- d]pyrimidin- 4(3H)-one | 8-(2-fluoro-4- (trifluoromethyl) phenyl)-2,3- dimethyl-6-(2- (2- methylpyridin- 4- yl)morpholino) pyrido[3,4- d]pyrimidin- 4(3H)-one | Similar specifications as mentioned in step-6 of example 1 Peak 1 Absolute stereochemistry unknown |
| Ex. 137B (Peak 2) 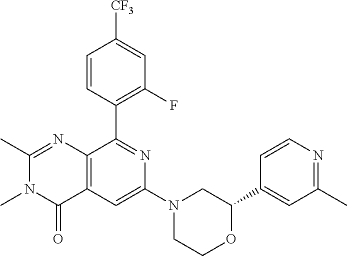 | (S)-8-(2-fluoro- 4- (trifluoromethyl) phenyl)-2,3- dimethyl-6-(2-(2- methylpyridin-4- yl)morpholino) pyrido[3,4- d]pyrimidin- 4(3H)-one | | Peak 2 Absolute stereochemistry unknown |
| Ex. 138A (Peak 1) 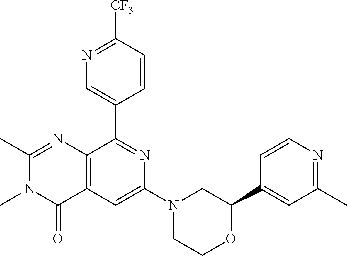 | (R)-2,3- dimethyl-6-(2-(2- methylpyridin-4- yl)morpholino)- 8-(6- (trifluoromethyl) pyridin-3- yl)pyrido[3,4- d]pyrimidin- 4(3H)-one | 2,3-dimethyl-6- (2-(2- methylpyridin-4- yl)morpholino)- 8-(6- (trifluoromethyl) pyridin-3- yl)pyrido[3,4- d]pyrimidin- 4(3H)-one | Similar specifications as mentioned in step-6 of example 1 Peak 1 Absolute stereochemistry unknown |
| Ex. 138B (Peak 2) 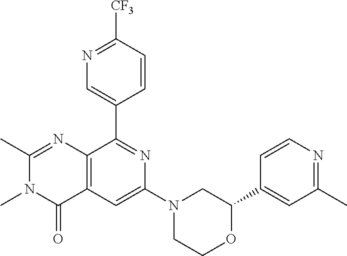 | (S)-2,3- dimethyl-6-(2-(2- methylpyridin-4- yl)morpholino)- 8-(6- (trifluoromethyl) pyridin-3- yl)pyrido[3,4- d]pyrimidin- 4(3H)-one | | Peak 2 Absolute stereochemistry unknown |
| Ex. 139A (Peak 1) 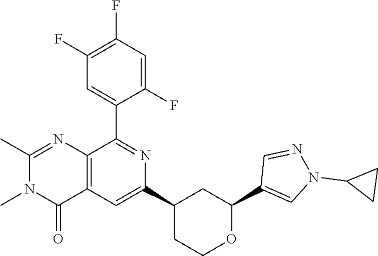 [SMILES: Cc1nc2c(-c3cc(F)c(F)cc3F)nc([C@@H]3CCO[C@H](c4cnn(C5CC5)c4)C3)cc2c(=O)n1C] | 6-((2S,4R)-2-(1- cyclopropyl-1H- pyrazol-4- yl)tetrahydro- 2H-pyran-4-yl)- 2,3-dimethyl-8- (2,4,5- trifluorophenyl) pyrido[3,4-d] pyrimidin- 4(3H)-one | 6-(2-(1- cyclopropyl- 1H-pyrazol-4- yl)tetrahydro- 2H-pyran-4- yl)-2,3- dimethyl-8- (2,4,5- trifluorophenyl) pyrido[3,4-d] pyrimidin- 4(3H)-one | Similar specifications as mentioned in step-6 of example 1 Peak 1 Absolute stereochemistry unknown |
| Ex. 139B (Peak 2) 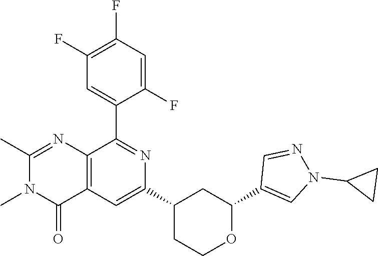 [SMILES: Cc1nc2c(-c3cc(F)c(F)cc3F)nc([C@H]3CCO[C@@H](c4cnn(C5CC5)c4)C3)cc2c(=O)n1C] | 6-((2R,4S)-2-(1- cyclopropyl-1H- pyrazol-4- yl)tetrahydro- 2H-pyran-4-yl)- 2,3-dimethyl-8- (2,4,5- trifluorophenyl) pyrido[3,4- d]pyrimidin- 4(3H)-one | | Peak 2 Absolute stereochemistry unknown |
| Ex. 140A (Peak 1) 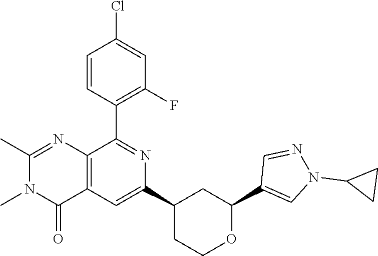 [SMILES: Cc1nc2c(-c3ccc(Cl)cc3F)nc([C@@H]3CCO[C@H](c4cnn(C5CC5)c4)C3)cc2c(=O)n1C] | 8-(4-chloro-2- fluorophenyl)-6- ((2S,4R)-2-(1- cyclopropyl-1H- pyrazol-4- yl)tetrahydro- 2H-pyran-4-yl)- 2,3- dimethylpyrido [3,4-d]pyrimidin- 4(3H)-one | 8-(4-chloro-2- fluorophenyl)- 6-2-(1- cyclopropyl- 1H-pyrazol-4- yl)tetrahydro- 2H-pyran-4- yl)-2,3- dimethylpyrido [3,4-d] pyrimidin- 4(3H)-one | Similar specifications as mentioned in step-6 of example 1 Peak 1 Absolute stereochemistry unknown |
| Ex. 140B (Peak 2) 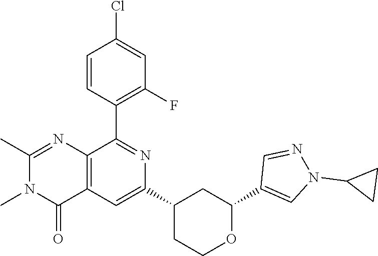 [SMILES: Cc1nc2c(-c3ccc(Cl)cc3F)nc([C@H]3CCO[C@@H](c4cnn(C5CC5)c4)C3)cc2c(=O)n1C] | 8-(4-chloro-2- fluorophenyl)-6- ((2R,4S)-2-(1- cyclopropyl-1H- pyrazol-4- yl)tetrahydro- 2H-pyran-4-yl)- 2,3- dimethylpyrido [3,4-d]pyrimidin- 4(3H)-one | | Peak 2 Absolute stereochemistry unknown |
| Ex. 141A (Peak 1) 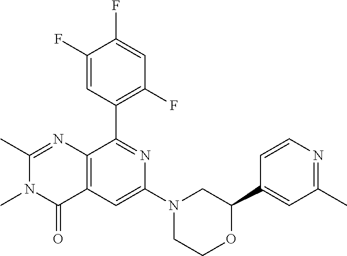 | (R)-2,3- dimethyl-6-(2-(2- methylpyridin-4- yl)morpholino)- 8-(2,4,5- trifluorophenyl) pyrido[3,4-d] pyrimidin- 4(3H)-one | 2,3-dimethyl-6- (2-(2- methylpyridin- 4-yl) morpholino)-8- (2,4,5- trifluorophenyl) pyrido[3,4-d] pyrimidin- 4(3H)-one | Similar specifications as mentioned in step-6 of example 1 Peak 1 Absolute stereochemistry unknown |
| Ex. 141B (Peak 2) 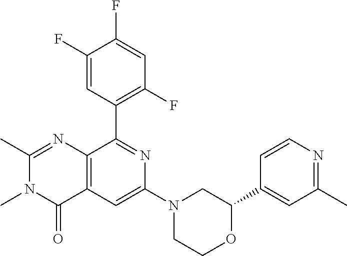 | (S)-2,3- dimethyl-6-(2-(2- methylpyridin-4- yl)morpholino)- 8-(2,4,5- trifluorophenyl) pyrido[3,4-d] pyrimidin- 4(3H)-one | | Peak 2 Absolute stereochemistry unknown |

Following table describes analytical data analysis and yield information of examples 125A-125B to examples 141A-141B:

| Compound | | | Analytical | | Yield |
|---|---|---|---|---|---|
| No. | HNMR | LCMS | chiral HPLC | Description | (%) |
| Ex. | 1H NMR (400 MHZ, DMSO- | Condition K: | CHIRALPAK IG | Yellow | 46.5 |
| 125A | d6) δ 7.74 (s, 1 H), 7.61-7.49 | Rt = 2.91 | (250 × 4.6) mm, | solid | |
| (Peak 1) | (m, 2 H), 7.46 (s, 1 H), 7.39 | min. m/z | 5 μ M.P.- | | |
| | (dd, J = 2.1, 8.4 Hz, 1 H), 7.31 | 469.3 | Hexane/DCM/ | | |
| | (s, 1 H), 4.55 (dd, J = 2.8, 10.4 | [M + H]⁺. | EtOH/IPAmine- | | |
| | Hz, 1 H), 4.29 (d, J = 12.4 Hz, | | 50/25/25/0.1 | | |
| | 1 H), 4.12 (d, J = 12.4 Hz, 1 H), | | Flow Rate-1.0 | | |
| | 4.04-3.99 (m, 1 H), 3.81 (s, | | ml/min | | |
| | 3 H), 3.77-3.66 (m, 1 H), 3.51 | | Solubility: | | |
| | (s, 3 H), 3.07-2.88 (m, 2 H), | | MeOH Rt = 9.34 | | |
| | 2.43 (s, 3 H). | | min for Peak 1 | | |
| Ex. | 1H NMR (400 MHZ, DMSO- | Condition K: | and Rt = 13.57 | Yellow | 46.3 |
| 125B | d6) δ 7.73 (s, 1 H), 7.61-7.49 | Rt = 2.91 | min for Peak 2. | solid | |
| (Peak 2) | (m, 2 H), 7.46 (s, 1 H), 7.39 | min. m/z | | | |
| | (dd, J = 2.2, 8.1 Hz, 1 H), 7.31 | 469.3 | | | |
| | (s, 1 H), 4.55 (dd, J = 2.7, 10.4 | [M + H]⁺. | | | |
| | Hz, 1 H), 4.29 (d, J = 12.2 Hz, | | | | |
| | 1 H), 4.12 (d, J = 12.5 Hz, 1 H), | | | | |
| | 4.05-3.96 (m, 1 H), 3.80 (s, | | | | |
| | 3 H), 3.77-3.66 (m, 1 H), 3.51 | | | | |
| | (s, 3 H), 3.07-2.88 (m, 2 H), | | | | |
| | 2.43 (s, 3 H). | | | | |
| Ex. | 1H NMR (400 MHZ, DMSO- | Condition K: | CHIRALPAK IG | Yellow | 35.7 |
| 126A | d6) δ 7.83-7.66 (m, 4 H), | Rt = 3.27 | (250 × 4.6) mm, | solid | |
| (Peak 1) | 7.46 (s, 1 H), 7.36 (s, 1 H), 4.55 | min. m/z | 5 μ M.P.- | | |
| | (d, J = 10.0 Hz, 1 H), 4.30 (d, J = | 503.2 | Hexane/DCM/ | | |
| | 12.2 Hz, 1 H), 4.13 (d, J = | [M + H]⁺. | EtOH/IPAmine- | | |
| | 12.2 Hz, 1 H), 4.02 (d, J = 10.7 | | 50/25/25/0.1 | | |
| | Hz, 1 H), 3.80 (s, 3 H), 3.71 (t, | | Flow Rate-1.0 | | |
| | J = 10.0 Hz, 1 H), 3.52 (s, 3 H), | | ml/min | | |
| | 3.08-2.89 (m, 2 H), 2.43 (s, | | Solubility: | | |
| | 3 H). | | MeOH Rt = 6.33 | | |
| Ex. | 1H NMR (400 MHZ, DMSO- | Condition K: | min for Peak 1 | Yellow | 35.7 |
| 126B | d6) δ 7.83-7.66 (m, 4 H), | Rt = 3.27 | and Rt = 7.77 | solid | |
| (Peak 2) | 7.46 (s, 1 H), 7.36 (s, 1 H), 4.55 | min. m/z | min for Peak 2. | | |
| | (d, J = 9.8 Hz, 1 H), 4.30 (d, J = | 503.2 | | | |
| | 12.4 Hz, 1 H), 4.13 (d, J = | [M + H]⁺. | | | |
| | 12.4 Hz, 1 H), 4.02 (d, J = 11.2 | | | | |
| | Hz, 1 H), 3.80 (s, 3 H), 3.71 (t, | | | | |
| | J = 10.1 Hz, 1 H), 3.52 (s, 3 H), | | | | |
| | 3.08-2.89 (m, 2 H), 2.43 (s, | | | | |
| | 3 H). | | | | |
| Ex. | 1H NMR (400 MHZ, DMSO- | Condition K: | CHIRALPAK IG | Yellow | 30.7 |
| 127A | d6) δ 9.51 (s, 1 H), 8.79 (d, J = | Rt = 3.14 | (250 × 4.6) mm, | solid | |
| (Peak 1) | 7.8 Hz, 1 H), 8.04 (d, J = 8.1 | min. m/z | 5 μ M.P.- | | |
| | Hz, 1 H), 7.76 (s, 1 H), 7.49 (s, | 486.2 | Hexane/DCM/ | | |
| | 1 H), 7.39 (s, 1 H), 4.64-4.51 | [M + H]⁺. | EtOH/IPAmine- | | |
| | (m, 1 H), 4.39 (d, J = 12.2 Hz, | | 50/25/25/0.1 | | |
| | 1 H), 4.22 (d, J = 12.7 Hz, 1 H), | | Flow Rate-1.0 | | |
| | 4.05 (d, J = 10.0 Hz, 1 H), 3.82 | | ml/min | | |
| | (s, 3 H), 3.79-3.69 (m, 1 H), | | Solubility: | | |
| | 3.54 (s, 3 H), 3.12-2.93 (m, | | MeOH Rt = 8.44 | | |
| | 2 H), 2.56 (s, 3 H). | | min for Peak 1 | | |
| Ex. | ¹H NMR (400 MHZ, DMSO-d₆) | Condition K: | and Rt = 9.87 | Yellow | 30.0 |
| 127B | δ 9.51 (s, 1 H), 8.86-8.72 | Rt = 3.14 | min for Peak 2. | solid | |
| (Peak 2) | (m, 1 H), 8.07-7.99 (m, 1 H), | min. m/z | | | |
| | 7.77 (s, 1 H), 7.49 (s, 1 H), 7.39 | 486.2 | | | |
| | (s, 1 H), 4.58 (d, J = 9.0 Hz, | [M + H]⁺. | | | |
| | 1 H), 4.39 (d, J = 12.4 Hz, 1 H), | | | | |
| | 4.22 (d, J = 12.3 Hz, 1 H), 4.05 | | | | |
| | (d, J = 10.6 Hz, 1 H), 3.82 (s, | | | | |
| | 3 H), 3.79-3.68 (m, 1 H), 3.54 | | | | |
| | (s, 3 H), 3.12-2.94 (m, 2 H), | | | | |
| | 2.56 (s, 3 H). | | | | |
| Ex. | ¹H NMR (400 MHZ, DMSO-d₆) | Condition K: | CHIRALPAK IG | Yellow | 46.5 |
| 128A | δ 8.42 (d, J = 5.0 Hz, 1 H), 7.61- | Rt = 2.57 | (250 × 4.6) mm, | solid | |
| (Peak 1) | 7.49 (m, 2 H), 7.44-7.35 (m, | min. m/z | 5 μ M.P.- | | |
| | 2 H), 7.31 (s, 1 H), 7.25 (d, J = | 506.3 | Hexane/DCM/ | | |
| | 4.8 Hz, 1 H), 4.78 (d, J = 8.8 Hz, | [M + H]⁺. | EtOH/IPAmine- | | |
| | 1 H), 4.50 (d, J = 11.9 Hz, 1 H), | | 50/25/25/0.1 | | |
| | 3.78 (d, J = 12.7 Hz, 1 H), 3.51 | | Flow Rate-1.0 | | |
| | (s, 3 H), 3.48 (s, 1 H), 2.90- | | ml/min. | | |
| | 2.79 (m, 1 H), 2.45 (d, J = 12.8 | | Solubility: | | |
| | Hz, 6 H), 1.00-0.83 (m, 2 H), | | MeOH Rt = 5.54 | | |
| | 0.78-0.62 (m, 2 H). | | min for Peak 1 | | |
| Ex. | ¹H NMR (400 MHZ, DMSO-d₆) | Condition K: | and Rt = 7.15 | Yellow | 46.3 |
| 128B | δ 8.42 (d, J = 5.0 Hz, 1 H), 7.61- | Rt = 2.57 | min for Peak 2. | solid | |
| (Peak 2) | 7.49 (m, 2 H), 7.44-7.35 (m, | min. m/z | | | |
| | 2 H), 7.31 (s, 1 H), 7.25 (d, J = | 506.3 | | | |
| | 4.8 Hz, 1 H), 4.78 (d, J = 8.8 Hz, | [M + H]⁺. | | | |
| | 1 H), 4.50 (d, J = 11.9 Hz, 1 H), | | | | |
| | 3.78 (d, J = 12.7 Hz, 1 H), 3.51 | | | | |
| | (s, 3 H), 3.48 (s, 1 H), 2.90- | | | | |
| | 2.79 (m, 1 H), 2.45 (d, J = 12.8 | | | | |
| | Hz, 6 H), 1.00-0.83 (m, 2 H), | | | | |
| | 0.78-0.62 (m, 2 H). | | | | |
| Ex. | ¹H NMR (400 MHZ, DMSO-d₆) | Condition K: | Chiralpak IG | Yellow | 39.7 |
| 129A | δ 7.74 (s, 1 H), 7.60 (q, J = 8.1 | Rt = 2.73 | (4.6 × 250 mm), | solid | |
| (Peak 1) | Hz, 1 H), 7.46 (s, 1 H), 7.38- | min. m/z | 5 μ | | |
| | 7.28 (m, 2 H), 7.19 (t, J = 7.1 | 453.3 | ARD/K/ | | |
| | Hz, 1 H), 4.55 (d, J = 10.1 Hz, | [M + H]⁺. | Mobile Phase: | | |
| | 1 H), 4.29 (d, J = 13.0 Hz, 1 H), | | Hexane/DCM/ | | |
| | 4.13 (d, J = 12.1 Hz, 1 H), 4.02 | | EtOH/IPAmine: | | |
| | (d, J = 10.8 Hz, 1 H), 3.80 (s, | | 50/25/25/0.1 | | |
| | 3 H), 3.71 (t, J = 11.0 Hz, 1 H), | | Flow Rate: 1.0 | | |
| | 3.51 (s, 3 H), 3.06-2.88 (m, | | ml/min | | |
| | 2 H), 2.44 (s, 3 H). | | Solubility: | | |
| Ex. | ¹H NMR (400 MHZ, DMSO-d₆) | Condition K: | MeOH Rt = | Yellow | 40.1 |
| 129B | δ 7.74 (s, 1 H), 7.60 (q, J = 8.1 | Rt = 2.73 | 10.14 min for | solid | |
| (Peak 2) | Hz, 1 H), 7.46 (s, 1 H), 7.38- | min. m/z | Peak 1 and Rt = | | |
| | 7.28 (m, 2 H), 7.19 (t, J = 7.1 | 453.3 | 15.06 min for | | |
| | Hz, 1 H), 4.55 (d, J = 10.1 Hz, | [M + H]⁺. | Peak 2. | | |
| | 1 H), 4.29 (d, J = 13.0 Hz, 1 H), | | | | |
| | 4.13 (d, J = 12.1 Hz, 1 H), 4.02 | | | | |
| | (d, J = 10.8 Hz, 1 H), 3.80 (s, | | | | |
| | 3 H), 3.71 (t, J = 11.0 Hz, 1 H), | | | | |
| | 3.51 (s, 3 H), 3.06-2.88 (m, | | | | |
| | 2 H), 2.44 (s, 3 H). | | | | |
| Ex. | ¹H NMR (400 MHZ, DMSO-d₆) | Condition K: | CHIRALPAK IG | Yellow | 42.3 |
| 130A | δ 7.74 (s, 1 H), 7.71-7.59 (m, | Rt = 2.80 | (250 × 4.6) mm, | solid | |
| (Peak 1) | 2 H), 7.46 (s, 1 H), 7.32 (s, 1 H), | min. m/z | 5 μ M.P.- | | |
| | 4.59-4.51 (m, 1 H), 4.29 (d, J = | 471.3 | Hexane/DCM/ | | |
| | 12.5 Hz, 1 H), 4.14 (d, J = | [M + H]⁺. | EtOH/IPAmine- | | |
| | 13.0 Hz, 1 H), 4.06-3.98 (m, | | 50/25/25/0.1 | | |
| | 1 H), 3.80 (s, 3 H), 3.76-3.66 | | Flow Rate-1.0 | | |
| | (m, 1 H), 3.51 (s, 3 H), 3.06- | | ml/min. | | |
| | 2.88 (m, 2 H), 2.44 (s, 3 H). | | Solubility: | | |
| Ex. | ¹H NMR (400 MHZ, DMSO-d₆) | Condition K: | MeOH Rt = 8.29 | Yellow | 41.4 |
| 130B | δ 7.74 (s, 1 H), 7.71-7.59 (m, | Rt = 2.80 | min for Peak 1 | solid | |
| (Peak 2) | 2 H), 7.46 (s, 1 H), 7.32 (s, 1 H), | min. m/z | and Rt = 11.85 | | |
| | 4.59-4.51 (m, 1 H), 4.29 (d, J = | 471.3 | min for Peak 2. | | |
| | 12.5 Hz, 1 H), 4.14 (d, J = | [M + H]⁺. | | | |
| | 13.0 Hz, 1 H), 4.06-3.98 (m, | | | | |
| | 1 H), 3.80 (s, 3 H), 3.76-3.66 | | | | |
| | (m, 1 H), 3.51 (s, 3 H), 3.06- | | | | |
| | 2.88 (m, 2 H), 2.44 (s, 3 H). | | | | |
| Ex. | ¹H NMR (400 MHZ, DMSO-d₆) | Condition | Chiralpak IG | Yellow | 38.7 |
| 131A | δ 7.83 (s, 1 H), 7.61-7.49 (m, | K: Rt = 2.97 | (4.6 × 250 mm), | Solid | |
| (Peak 1) | 2 H), 7.46 (s, 1 H), 7.39 (dd, J = | min. m/z | 5 μ | | |
| | 2.0, 8.1 Hz, 1 H), 7.32 (s, 1 H), | 495.3 | ARD/K/ | | |
| | 4.57-4.49 (m, 1 H), 4.28 (d, J | [M + H]⁺. | Mobile Phase: | | |
| | = 12.8 Hz, 1 H), 4.14 (d, J = | | Hexane/DCM/ | | |
| | 12.8 Hz, 1 H), 4.08-3.96 (m, | | EtOH/IPAmine: | | |
| | 1 H), 3.76-3.63 (m, 2 H), 3.51 | | 50/25/25/0.1 | | |
| | (s, 3 H), 3.06-2.89 (m, 2 H), | | Flow Rate: 1.0 | | |
| | 2.43 (s, 3 H), 1.05-0.97 (m, | | ml/min | | |
| | 2 H), 1.00-0.88 (m, 2 H). | | Solubility: | | |
| Ex. | ¹H NMR (400 MHZ, DMSO-d₆) | Condition K: | MeOH Rt = | Yellow | 39.4 |
| 131B | δ 7.83 (s, 1 H), 7.61-7.49 (m, | Rt = 2.97 | 10.06 min for | Solid | |
| (Peak 2) | 2 H), 7.46 (s, 1 H), 7.39 (dd, J = | min. m/z | Peak 1 and Rt = | | |
| | 2.0, 8.1 Hz, 1 H), 7.32 (s, 1 H), | 495.3 | 13.73 min for | | |
| | 4.57-4.49 (m, 1 H), 4.28 (d, J = | [M + H]⁺. | Peak 2. | | |
| | 12.8 Hz, 1 H), 4.14 (d, J = | | | | |
| | 12.8 Hz, 1 H), 4.08-3.96 (m, | | | | |
| | 1 H), 3.76-3.63 (m, 2 H), 3.51 | | | | |
| | (s, 3 H), 3.06-2.89 (m, 2 H), | | | | |
| | 2.43 (s, 3 H), 1.05-0.97 (m, | | | | |
| | 2 H), 1.00-0.88 (m, 2 H). | | | | |
| Ex. | ¹H NMR (400 MHZ, DMSO-d₆) | Condition K: | CHIRALPAK IG | Yellow | 41.5 |
| 132A | δ 7.83 (s, 1 H), 7.78 (t, J = 6.8 | Rt = 3.06 | (250 × 4.6) mm, | Solid | |
| (Peak 1) | Hz, 2 H), 7.69 (d, J = 7.9 Hz, | min. m/z | 5 μ M.P.- | | |
| | 1 H), 7.46 (s, 1 H), 7.36 (s, 1 H), | 529.3 | Hexane/DCM/ | | |
| | 4.58-4.49 (m, 1 H), 4.29 (d, J = | [M + H]⁺. | EtOH/IPAmine- | | |
| | 12.9 Hz, 1 H), 4.14 (d, J = | | 50/25/25/0.1 | | |
| | 12.1 Hz, 1 H), 4.05-3.97 (m, | | Flow Rate-1.0 | | |
| | 1 H), 3.76-3.63 (m, 2 H), 3.52 | | ml/min | | |
| | (s, 3 H), 3.06-2.90 (m, 2 H), | | Solubility: | | |
| | 2.43 (s, 3 H), 1.06-0.88 (m, | | MeOH Rt = 5.82 | | |
| | 4 H). | | min for Peak 1 | | |
| Ex. | ¹H NMR (400 MHZ, DMSO-d₆) | Condition K: | and Rt = 6.69 | Yellow | 40.9 |
| 132B | δ 7.83 (s, 1 H), 7.78 (t, J = 6.8 | Rt = 3.06 | min for Peak 2 | Solid | |
| (Peak 2) | Hz, 2 H), 7.69 (d, J = 7.9 Hz, | min. m/z | | | |
| | 1 H), 7.46 (s, 1 H), 7.36 (s, 1 H), | 529.4 | | | |
| | 4.58-4.49 (m, 1 H), 4.29 (d, J = | [M + H]⁺. | | | |
| | 12.9 Hz, 1 H), 4.14 (d, J = | | | | |
| | 12.1 Hz, 1 H), 4.05-3.97 (m, | | | | |
| | 1 H), 3.76-3.63 (m, 2 H), 3.52 | | | | |
| | (s, 3 H), 3.06-2.90 (m, 2 H), | | | | |
| | 2.43 (s, 3 H), 1.06-0.88 (m, | | | | |
| | 4 H). | | | | |
| Ex. | ¹H NMR (400 MHZ, DMSO-d₆) | Condition K: | Chiralpak IG | Yellow | 37.7 |
| 133A | δ 7.83 (s, 1 H), 7.70-7.58 (m, | Rt = 2.89 | (4.6 × 250 mm), | solid | |
| (Peak 1) | 2 H), 7.46 (s, 1 H), 7.33 (s, 1 H), | min. m/z | 5 μ | | |
| | 4.53 (d, J = 9.5 Hz, 1 H), 4.28 | 497.3 | ARD/K/ | | |
| | (d, J = 12.6 Hz, 1 H), 4.15 (d, J = | [M + H]⁺. | Mobile Phase: | | |
| | 12.0 Hz, 1 H), 4.01 (d, J = | | Hexane/DCM/ | | |
| | 10.8 Hz, 1 H), 3.81-3.61 (m, | | EtOH/IPAmine: | | |
| | 2 H), 3.52 (s, 3 H), 3.05-2.89 | | 50/25/25/0.1 | | |
| | (m, 2 H), 2.44 (s, 3 H), 1.12- | | Flow Rate: 1.0 | | |
| | 0.81 (m, 4 H). | | ml/min | | |
| Ex. | ¹H NMR (400 MHZ, DMSO-d₆) | Condition K: | Solubility: | Yellow | 37.0 |
| 133B | δ 7.83 (s, 1 H), 7.70-7.58 (m, | Rt = 2.89 | MeOH | Solid | |
| (Peak 2) | 2 H), 7.46 (s, 1 H), 7.33 (s, 1 H), | min. m/z | Rt = 8.74 min for | | |
| | 4.53 (d, J = 9.5 Hz, 1 H), 4.28 | 497.3 | Peak 1 and Rt = | | |
| | (d, J = 12.6 Hz, 1 H), 4.15 (d, J = | [M + H]⁺. | 12.33 min for | | |
| | 12.0 Hz, 1 H), 4.01 (d, J = | | Peak 2 | | |
| | 10.8 Hz, 1 H), 3.81-3.61 (m, | | | | |
| | 2 H), 3.52 (s, 3 H), 3.05-2.89 | | | | |
| | (m, 2 H), 2.44 (s, 3 H), 1.12- | | | | |
| | 0.81 (m, 4 H). | | | | |
| Ex. | ¹H NMR (400 MHZ, DMSO-d₆) | Condition K: | CHIRALPAK IG | Yellow | 46.5 |
| 134A | δ 7.77 (t, J = 7.6 Hz, 2 H), 7.70 | Rt = 3.08 | (250 × 4.6) mm, | Solid | |
| (Peak 1) | (d, J = 6.4 Hz, 2 H), 7.40 (d, J = | min. m/z | 5 μ M.P.- | | |
| | 27.7 Hz, 2 H), 4.87-4.79 (m, | 531.4 | Hexane/DCM/ | | |
| | 1 H), 4.38 (d, J = 11.7 Hz, 1 H), | [M + H]⁺. | EtOH/IPAmine- | | |
| | 4.16 (d, J = 12.7 Hz, 1 H), 3.80 | | 50/25/25/0.1 | | |
| | (s, 3 H), 3.51 (s, 3 H), 2.86- | | Flow Rate-1.0 | | |
| | 2.75 (m, 2 H), 2.43 (s, 3 H), 1.27 | | ml/min | | |
| | (d, J = 18.9 Hz, 6 H). | | Solubility: | | |
| Ex. | ¹H NMR (400 MHZ, DMSO-d₆) | Condition K: | MeOH | Yellow | 46.3 |
| 134B | δ 7.77 (t, J = 7.6 Hz, 2 H), 7.70 | Rt = 3.09 | Rt = 4.57 min for | Solid | |
| (Peak 2) | (d, J = 6.4 Hz, 2 H), 7.40 (d, J = | min. m/z | Peak 1 and Rt = | | |
| | 27.7 Hz, 2 H), 4.87-4.79 (m, | 531.4 | 5.14 min for | | |
| | 1 H), 4.38 (d, J = 11.7 Hz, 1 H), | [M + H]⁺. | Peak 2 | | |
| | 4.16 (d, J = 12.7 Hz, 1 H), 3.80 | | | | |
| | (s, 3 H), 3.51 (s, 3 H), 2.86- | | | | |
| | 2.75 (m, 2 H), 2.43 (s, 3 H), 1.27 | | | | |
| | (d, J = 18.9 Hz, 6 H). | | | | |
| Ex. | ¹H NMR (400 MHZ, Methanol- | Condition K: | CHIRALPAK IG | Yellow | 34.0 |
| 135A | d₄) δ 8.39 (d, J = 5.2 Hz, 1 H), | Rt = 2.52 | (250 × 4.6) mm, | Solid | |
| (Peak 1) | 7.54 (t, J = 8.0 Hz, 1 H), 7.40 (d, | min. m/z | 5 μ M.P.- | | |
| | J = 8.9 Hz, 2 H), 7.36-7.24 (m, | 480.3 | Hexane/DCM/ | | |
| | 3 H), 4.73-4.61 (m, 1 H), 4.49 | [M + H]⁺. | EtOH/IPAmine- | | |
| | (d, J = 12.5 Hz, 1 H), 4.27- | | 50/25/25/0.1 | | |
| | 4.15 (m, 2 H), 3.94-3.82 (m, | | Flow Rate-1.0 | | |
| | 1 H), 3.60 (s, 3 H), 3.19-3.07 | | ml/min. | | |
| | (m, 1 H), 2.88-2.77 (m, 1 H), | | Solubility: | | |
| | 2.52 (d, J = 14.7 Hz, 6 H). | | MeOH | | |
| Ex. | ¹H NMR (400 MHZ, Methanol- | Condition K: | Rt = 6.36 min for | Yellow | 36.2 |
| 135B | d₄) δ 8.39 (d, J = 5.2 Hz, 1 H), | Rt = 2.52 | Peak 1 and Rt = | Solid | |
| (Peak 2) | 7.54 (t, J = 8.0 Hz, 1 H), 7.40 (d, | min. m/z | 9.55 min for | | |
| | J = 8.9 Hz, 2 H), 7.36-7.24 (m, | 480.3 | Peak 2 | | |
| | 3 H), 4.73-4.61 (m, 1 H), 4.49 | [M + H]⁺. | | | |
| | (d, J = 12.5 Hz, 1 H), 4.27- | | | | |
| | 4.15 (m, 2 H), 3.94-3.82 (m, | | | | |
| | 1 H), 3.60 (s, 3 H), 3.19-3.07 | | | | |
| | (m, 1 H), 2.88-2.77 (m, 1 H), | | | | |
| | 2.52 (d, J = 14.7 Hz, 6 H). | | | | |
| Ex. | ¹H NMR (400 MHZ, DMSO-d₆) | Condition K: | CHIRALPAK IG | Off | 35.7 |
| 136A | δ 8.43 (d, J = 5.0 Hz, 1 H), 7.60 | Rt = 2.21 | (250 × 4.6) mm, | White | |
| (Peak 1) | (q, J = 8.2 Hz, 1 H), 7.41-7.25 | min. m/z | 5 μ M.P.- | Solid | |
| | (m, 4 H), 7.24-7.15 (m, 1 H), | 464.3 | Hexane/DCM/ | | |
| | 4.67-4.61 (m, 1 H), 4.38 (d, J = | [M + H]⁺. | EtOH/IPAmine- | | |
| | 12.5 Hz, 1 H), 4.22 (d, J = | | 50/25/25/0.1 | | |
| | 12.3 Hz, 1 H), 4.18-4.11 (m, | | Flow Rate-1.0 | | |
| | 1 H), 3.84-3.73 (m, 1 H), 3.51 | | ml/min Solubility: | | |
| | (s, 3 H), 3.09-2.97 (m, 1 H), | | MeOH | | |
| | 2.83-2.72 (m, 1 H), 2.48 (s, | | Rt = 6.07 min for | | |
| | 3 H), 2.44 (s, 3 H). | | Peak 1 and Rt = | | |
| Ex. | ¹H NMR (400 MHZ, DMSO-d₆) | Condition K: | 10.12 min for | Off | 34.7 |
| 136B | δ 8.43 (d, J = 5.0 Hz, 1 H), 7.60 | Rt = 2.19 | Peak 2 | White | |
| (Peak 2) | (q, J = 8.2 Hz, 1 H), 7.41-7.25 | min. m/z | | Solid | |
| | (m, 4 H), 7.24-7.15 (m, 1 H), | 464.3 | | | |
| | 4.67-4.61 (m, 1 H), 4.38 (d, J = | [M + H]⁺. | | | |
| | 12.5 Hz, 1 H), 4.22 (d, J = | | | | |
| | 12.3 Hz, 1 H), 4.18-4.11 (m, | | | | |
| | 1 H), 3.84-3.73 (m, 1 H), 3.51 | | | | |
| | (s, 3 H), 3.09-2.97 (m, 1 H), | | | | |
| | 2.83-2.72 (m, 1 H), 2.48 (s, | | | | |
| | 3 H), 2.44 (s, 3 H). | | | | |
| Ex. | ¹H NMR (400 MHZ, DMSO-d₆) | Condition K: | Chiralpak IG | Yellow | 30.7 |
| 137A | δ 8.43 (d, J = 5.2 Hz, 1 H), 7.78 | Rt = 2.56 | (4.6 × 250 mm), | Solid | |
| (Peak 1) | (t, J = 7.7 Hz, 2 H), 7.70 (d, J = | min. m/z | 5 μ | | |
| | 8.2 Hz, 1 H), 7.41 (s, 1 H), 7.34 | 514.3 | ARD/K/ | | |
| | (s, 1 H), 7.27 (d, J = 4.8 Hz, | [M + H]⁺. | Mobile Phase: | | |
| | 1 H), 4.64 (d, J = 11.1 Hz, 1 H), | | Hexane/DCM/ | | |
| | 4.40 (d, J = 11.9 Hz, 1 H), 4.23 | | EtOH/IPAmine: | | |
| | (d, J = 12.4 Hz, 1 H), 4.14 (d, J = | | 50/25/25/0.1 | | |
| | 9.8 Hz, 1 H), 3.78 (t, J = 9.8 | | Flow Rate: 1.0 | | |
| | Hz, 1 H), 3.52 (s, 3 H), 3.03 (t, J = | | ml/min | | |
| | 9.4 Hz, 1 H), 2.78 (t, J = 11.2 | | Solubility: | | |
| | Hz, 1 H), 2.48 (s, 3 H), 2.43 (s, | | MeOH. | | |
| | 3 H). | | Rt = 5.02 min for | | |
| Ex. | ¹H NMR (400 MHZ, DMSO-d₆) | Condition K: | Peak 1 and Rt = | Yellow | 30.0 |
| 137B | δ 8.43 (d, J = 5.2 Hz, 1 H), 7.78 | Rt = 2.56 | 5.89 min for | Solid | |
| (Peak 2) | (t, J = 7.7 Hz, 2 H), 7.70 (d, J = | min. m/z | Peak 2 | | |
| | 8.2 Hz, 1 H), 7.41 (s, 1 H), 7.34 | 514.3 | | | |
| | (s, 1 H), 7.27 (d, J = 4.8 Hz, | [M + H]⁺. | | | |
| | 1 H), 4.64 (d, J = 11.1 Hz, 1 H), | | | | |
| | 4.40 (d, J = 11.9 Hz, 1 H), 4.23 | | | | |
| | (d, J = 12.4 Hz, 1 H), 4.14 (d, J = | | | | |
| | 9.8 Hz, 1 H), 3.78 (t, J = 9.8 | | | | |
| | Hz, 1 H), 3.52 (s, 3 H), 3.03 (t, J = | | | | |
| | 9.4 Hz, 1 H), 2.78 (t, J = 11.2 | | | | |
| | Hz, 1 H), 2.48 (s, 3 H), 2.43 (s, | | | | |
| | 3 H). | | | | |
| Ex. | ¹H NMR (400 MHZ, Methanol- | Condition K: | Chiralpak IG | Yellow | 39.8 |
| 138A | d₄) δ 9.51 (s, 1 H), 8.85 (d, J = | Rt = 2.21 | (4.6 × 250 mm), | Solid | |
| (Peak 1) | 8.0 Hz, 1 H), 8.42 (d, J = 5.1 Hz, | min. m/z | 5 μ | | |
| | 1 H), 7.91 (d, J = 8.1 Hz, 1 H), | 497.3 | ARD/K/ | | |
| | 7.47 (s, 1 H), 7.44-7.36 (m, | [M + H]⁺. | Mobile Phase: | | |
| | 2 H), 4.69 (d, J = 10.1 Hz, 1 H), | | Hexane/DCM/ | | |
| | 4.54 (d, J = 12.9 Hz, 1 H), 4.30 | | EtOH/IPAmine: | | |
| | (d, J = 12.7 Hz, 1 H), 4.22 (d, J = | | 50/25/25/0.1 | | |
| | 10.8 Hz, 1 H), 3.94-3.84 (m, | | Flow Rate: 1.0 | | |
| | 1 H), 3.62 (s, 3 H), 3.22-3.06 | | ml/min | | |
| | (m, 1 H), 2.85 (t, J = 11.0 Hz, | | Solubility: | | |
| | 1 H), 2.58 (d, J = 6.0 Hz, 6 H). | | MeOH | | |
| Ex. | ¹H NMR (400 MHZ, Methanol- | Condition K: | Rt = 6.54 min for | Yellow | 40.3 |
| 138B | d₄) δ 9.51 (s, 1 H), 8.85 (d, J = | Rt = 2.21 | Peak 1 and Rt = | Solid | |
| (Peak 2) | 8.0 Hz, 1 H), 8.42 (d, J = 5.1 Hz, | min. m/z | 7.90 min for | | |
| | 1 H), 7.91 (d, J = 8.1 Hz, 1 H), | 497.3 | Peak 2 | | |
| | 7.47 (s, 1 H), 7.44-7.36 (m, | [M + H]⁺. | | | |
| | 2 H), 4.69 (d, J = 10.1 Hz, 1 H), | | | | |
| | 4.54 (d, J = 12.9 Hz, 1 H), 4.30 | | | | |
| | (d, J = 12.7 Hz, 1 H), 4.22 (d, J = | | | | |
| | 10.8 Hz, 1 H), 3.94-3.84 (m, | | | | |
| | 1 H), 3.62 (s, 3 H), 3.22-3.06 | | | | |
| | (m, 1 H), 2.85 (t, J = 11.0 Hz, | | | | |
| | 1 H), 2.58 (d, J = 6.0 Hz, 6 H). | | | | |
| Ex. | ¹H NMR (400 MHZ, DMSO-d₆) | Condition K: | CHIRALCEL | White | 42.5 |
| 139A | δ 7.91 (s, 1 H), 7.75-7.62 (m, | Rt = 2.83 | OX-H | solid | |
| (Peak 1) | 3 H), 7.37 (s, 1 H), 4.46 (d, J = | min. m/z | CoSolvent 0.3% | | |
| | 10.9 Hz, 1 H), 4.16-3.98 (m, | 496.3 | IPAmine_MeOH | | |
| | 1 H), 3.72-3.59 (m, 2 H), 3.55 | [M + H]⁺. | Solubility: | | |
| | (s, 3 H), 3.30-3.10 (m, 1 H), | | MeOH | | |
| | 2.69-2.41 (m, 3 H), 2.15 (d, J = | | Rt = 3.10 min for | | |
| | 12.6 Hz, 1 H), 1.91-1.74 (m, | | Peak 1 and Rt = | | |
| | 3 H), 1.09-0.81 (m, 4 H). | | 3.33 min for | | |
| Ex. | ¹H NMR (400 MHZ, DMSO-d₆) | Condition K: | Peak 2 | White | 41.3 |
| 139B | δ 7.91 (s, 1 H), 7.75-7.62 (m, | Rt = 2.83 | | solid | |
| (Peak 2) | 3 H), 7.37 (s, 1 H), 4.46 (d, J = | min. m/z | | | |
| | 10.9 Hz, 1 H), 4.16-3.98 (m, | 496.3 | | | |
| | 1 H), 3.72-3.59 (m, 2 H), 3.55 | [M + H]⁺. | | | |
| | (s, 3 H), 3.30-3.10 (m, 1 H), | | | | |
| | 2.69-2.41 (m, 3 H), 2.15 (d, J = | | | | |
| | 12.6 Hz, 1 H), 1.91-1.74 (m, | | | | |
| | 3 H), 1.09-0.81 (m, 4 H). | | | | |
| Ex. | ¹H NMR (400 MHZ, DMSO-d₆) | Condition K: | Chiralpak IG | White | 42.3 |
| 140A | δ 7.43 (d, J = 8.0 Hz, 1 H), 7.37 | Rt = 2.90 | (4.6 × 250 mm), | solid | |
| (Peak 1) | (s, 1 H), 3.32-3.15 (m, 4 H), | min. m/z | 5 μ | | |
| | 2.14 (d, J = 12.8 Hz, 1 H), 2.03- | 494.3 | ARD/K/ | | |
| | 1.70 (m, 3 H), 1.05-0.83 (m, | [M + H]⁺. | Mobile Phase: | | |
| | 4 H), 3.32-3.23 (m, 6 H), 7.90 | | Hexane/DCM/ | | |
| | (s, 1 H), 7.71 (s, 1 H), 7.59 (d, | | EtOH/IPAmine: | | |
| | J = 8.0 Hz, 2 H), 4.46 (d, J = 10.7 | | 40/30/30/0.1 | | |
| | Hz, 1 H), 4.11-4.02 (m, 1 H), | | Flow Rate: 1.0 | | |
| | 3.72-3.59 (m, 2 H), 3.54 (s, | | ml/min | | |
| | 3 H). | | Solubility: | | |
| Ex. | ¹H NMR (400 MHZ, DMSO-d₆) | Condition K: | MeOH | White | 41.8 |
| 140B | δ 7.43 (d, J = 8.0 Hz, 1 H), 7.37 | Rt = 2.90 | Rt = 7.43 min for | solid | |
| (Peak 2) | (s, 1 H), 3.32-3.15 (m, 4 H), | min. m/z | Peak 1 and Rt = | | |
| | 2.14 (d, J = 12.8 Hz, 1 H), 2.03- | 494.3 | 10.21 min for | | |
| | 1.70 (m, 3 H), 1.05-0.83 (m, | [M + H]⁺. | Peak 2 | | |
| | 4 H), 3.32-3.23 (m, 6 H), 7.90 | | | | |
| | (s, 1 H), 7.71 (s, 1 H), 7.59 (d, | | | | |
| | J = 8.0 Hz, 2 H), 4.46 (d, J = 10.7 | | | | |
| | Hz, 1 H), 4.11-4.02 (m, 1 H), | | | | |
| | 3.72-3.59 (m, 2 H), 3.54 (s, | | | | |
| | 3 H). | | | | |
| Ex. | 1H NMR (400 MHZ, DMSO-d6) | Condition K: | CHIRALPAK IG | Yellow | 42.1 |
| 141A | δ 8.44 (d, J = 5.0 Hz, 1 H), 7.73- | Rt = 2.44 | (250 × 4.6) mm, | solid | |
| (Peak 1) | 7.58 (m, 2 H), 7.39 (s, 1 H), | min. m/z | 5 μ M.P.- | | |
| | 7.35 (s, 1 H), 7.28 (d, J = 4.9 | 482.3 | Hexane/DCM/ | | |
| | Hz, 1 H), 4.68-4.59 (m, 1 H), | [M + H]⁺. | EtOH/IPAmine- | | |
| | 4.39 (d, J = 12.0 Hz, 1 H), 4.23 | | 50/25/25/0.1 | | |
| | (d, J = 12.6 Hz, 1 H), 4.19- | | Flow Rate-1.0 | | |
| | 4.10 (m, 1 H), 3.84-3.72 (m, | | ml/min | | |
| | 1 H), 3.52 (s, 3 H), 3.09-2.97 | | Solubility: | | |
| | (m, 1 H), 2.84-2.73 (m, 1 H), | | MeOH | | |
| | 2.48 (s, 4 H), 2.45 (s, 3 H). | | Rt = 5.81 min for | | |
| Ex. | 1H NMR (400 MHZ, DMSO-d6) | Condition K: | Peak 1 and Rt = | Yellow | 39.8 |
| 141B | δ 8.44 (d, J = 5.0 Hz, 1 H), 7.73- | Rt = 2.44 | 8.98 min for | solid | |
| (Peak 2) | 7.58 (m, 2 H), 7.39 (s, 1 H), | min. m/z | Peak 2 | | |
| | 7.35 (s, 1 H), 7.28 (d, J = 4.9 | 482.3 | | | |
| | Hz, 1 H), 4.68-4.59 (m, 1 H), | [M + H]⁺. | | | |
| | 4.39 (d, J = 12.0 Hz, 1 H), 4.23 | | | | |
| | (d, J = 12.6 Hz, 1 H), 4.19- | | | | |
| | 4.10 (m, 1 H), 3.84-3.72 (m, | | | | |
| | 1 H), 3.52 (s, 3 H), 3.09-2.97 | | | | |
| | (m, 1 H), 2.84-2.73 (m, 1 H), | | | | |
| | 2.48 (s, 4 H), 2.45 (s, 3 H). | | | | |

### Example 142A—Peak1

### (R)8-(4-chloro-2-fluorophenyl)-6-(2,2-dimethyl-6-(1-methyl-1H-pyrazol-4-yl)morpholino)-2,3-dimethylpyrido[3,2-d]pyrimidin-4 (3H)-one

### Example 142B—Peak2

### (S)8-(4-chloro-2-fluorophenyl)-6-(2,2-dimethyl-6-(1-methyl-1H-pyrazol-4-yl)morpholino)-2,3-dimethylpyrido[3,2-d]pyrimidin-4 (3H)-one

### Step-1—Preparation of methyl methyl 3-amino-4-bromo-6-chloropyridine-2-carboxylate

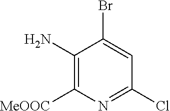 [SMILES: COC(=O)c1nc(Cl)cc(Br)c1N]

To a stirred solution of methyl 3-amino-6-chloropicolinate (1.0 gm, 5.38 mmol) in DMF (5.0 mL), NBS (1.43 gm, 8.06 mmol) was added at 0° C. Resulting mixture was heated at 80° C. for 2 h. Reaction mixture was quenched with crushed ice, precipitate thus obtained was filtered, washed with cold water, and dried to afford methyl 3-amino-4-bromo-6-chloropyridine-2-carboxylate (1.2 g, 84.07%) as off white solid.

LCMS Condition A: Rt=1.93 min. m/z 265.1 [M+H]⁺.

### Step-2—Preparation of methyl 4-bromo-6-chloro-3-acetamidopyridine-2-carboxylate

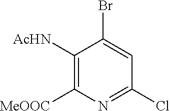 [SMILES: COC(=O)c1nc(Cl)cc(Br)c1NC(C)=O]

To a stirred solution of methyl 3-amino-4-bromo-6-chloropyridine-2-carboxylate (1.2 gm, 4.55 mmol) in DMF (10.0 mL), K₂CO₃ (1.26 gm, 9.09 mmol) was added portion wise under argon atmosphere. Resulting mixture was heated at 80° C. for 2 h. It was then cooled to RT and acetyl chloride (0.65 mL) was added dropwise at 0° C. and stirred at RT for 16 h. Reaction mixture was quenched with cold water and extracted with ethyl acetate. Combined organic layer was dried over anhydrous Na₂SO₄ filtered and concentrated. Crude mass was purified by combi flash chromatography using 30%-50% ethyl acetate in hexane to afford methyl 4-bromo-6-chloro-3-acetamidopyridine-2-carboxylate (800.0 mg, 57.21%) as off white solid.

LCMS Condition A: Rt=1.67 min. m/z 307.1 [M+H]⁺.

### Step-3—Preparation of 4-bromo-6-chloro-3-acetamidopyridine-2-carboxylic acid

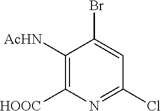 [SMILES: CC(=O)Nc1c(Br)cc(Cl)nc1C(=O)O]

To a stirred solution of methyl 4-bromo-6-chloro-3-acetamidopyridine-2-carboxylate (800 mg, 2.62 mmol) in THF (10.0 mL) and H2O (3 mL), LiOH·H₂O (275.14 mg, 6.56 mmol) was added. Resulting mixture was stirred at RT for 1 h. Then reaction mixture was evaporated, diluted with water, acidified with 1N HCl and extracted with 10% MeOH-DCM. The organic layer concentrated under reduced pressure to get 4-bromo-6-chloro-3-acetamidopyridine-2-carboxylic acid (650.0 mg, 84.43%) as yellow solid.

LCMS Condition A: Rt=0.87 min. m/z 293.1 [M+H]⁺.

### Step-4—Preparation of 8-bromo-6-chloro-2,3-dimethyl-3H,4H-pyrido[3,2-d]pyrimidin-4-one

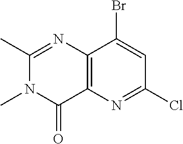

To a stirred solution of 4-bromo-6-chloro-3-acetamidopyridine-2-carboxylic acid (400 mg, 1.365 mmol) in pyridine (5.0 mL), MeNH₂ in THF (2M) (4.0 ml, 8.191 mmol) was added at 0° C. under argon atmosphere. Then the reaction was stirred at RT for 15 min and T₃P (50% in EA) (2.0 ml, 2.73 mmol) was added at cooling. Then the reaction was stirred at 80° C. for 48 h. Excess pyridine of the reaction mixture was evaporated, then reaction mixture was quenched with water, extracted with ethyl acetate. Organic portion was dried over Na2SO4 and concentrated. The crude mass was purified by combi flash chromatography using 20%-50% Ethyl acetate in Hexane to afford 8-bromo-6-chloro-2,3-dimethyl-3H,4H-pyrido[3,2-d]pyrimidin-4-one (100 mg, 25.39%) as white solid.

LCMS Condition A: Rt=2.57 min. m/z 288.1 [M+H]⁺.

### Step-5—Preparation of 6-chloro-8-(4-chloro-2-fluorophenyl)-2,3-dimethyl-3H,4H-pyrido[3,2-d]pyrimidin-4-one

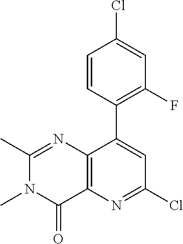 [SMILES: Cc1nc2c(-c3ccc(Cl)cc3F)cc(Cl)nc2c(=O)n1C]

To a stirred solution of 8-bromo-6-chloro-2,3-dimethyl-3H,4H-pyrido[3,2-d]pyrimidin-4-one (100.0 mg, 0.35 mmol), (4-chloro-2-fluorophenyl)boronic acid (54.46 gm, 0.31 mmol) in Dioxane:Water (4:1) ml was added Na₂CO₃ (73.54 mg, 0.69 mmol). The reaction mass was degassed under argon atmosphere over 10 minutes. Then PdCl2(dppf) (25.4 mg, 0.04 mmol) was added to this reaction and the resultant reaction mass was heated at 80° C. for 1 h. Reaction mixture was quenched with water, extracted with ethyl acetate. Combined organic layer was dried over Na2SO4 and concentrated.

The crude was purified by combi flash chromatography using 30%-50% ethyl acetate in hexane to afford 6-chloro-8-(4-chloro-2-fluorophenyl)-2,3-dimethyl-3H,4H-pyrido[3,2-d]pyrimidin-4-one (60.0 mg, 51.1%) as off white solid.

LCMS Condition A: Rt=2.14 min. m/z 338.2 [M+H]⁺.

### Step-6—Preparation of 8-(4-chloro-2-fluorophenyl)-6-[2,2-dimethyl-6-(1-methyl-1H-pyrazol-4-yl)morpholin-4-yl]-2,3-dimethyl-3H,4H-pyrido[3,2-d]pyrimidin-4-one

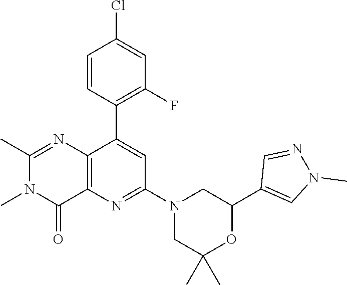

To a solution of 6-chloro-8-(4-chloro-2-fluorophenyl)-2,3-dimethyl-3H,4H-pyrido[3,2-d]pyrimidin-4-one (100 mg, 0.3 mmol) and 2,2-dimethyl-6-(1-methyl-1H-pyrazol-4-yl)morpholine (86.61 mg, 0.44 mmol) in DMSO was added DIPEA (0.103 ml, 0.59 mmol) and stirred at 120° C. for 16 hrs. The crude mass was purified by prep-HPLC chromatography to get 8-(4-chloro-2-fluorophenyl)-6-[2,2-dimethyl-6-(1-methyl-1H-pyrazol-4-yl)morpholin-4-yl]-2,3-dimethyl-3H,4H-pyrido[3,2-d]pyrimidin-4-one (44 mg, 29.94%) as off white solid.

Preparative HPLC method:

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 40% A and 60% B in 3 min, then to 10% A and 90% B in 20 min., then to 5% A and 95% B in 21 min., held this composition up to 22 min. for column washing, then returned to initial composition in 23 min. and held till 25 min.

LCMS Condition K: Rt=2.80 min. m/z 497.3 [M+H]⁺.

### Step-7—Preparation of (R) 8-(4-chloro-2-fluorophenyl)-6-[2,2-dimethyl-6-(1-methyl-1H-pyrazol-4-yl)morpholin-4-yl]-2,3-dimethyl-3H,4H-pyrido[3,2-d]pyrimidin-4-one [Example 142A] and (S) 8-(4-chloro-2-fluorophenyl)-6-[2,2-dimethyl-6-(1-methyl-1H-pyrazol-4-yl)morpholin-4-yl]-2,3-dimethyl-3H,4H-pyrido[3,2-d]pyrimidin-4-one [Example 142B]

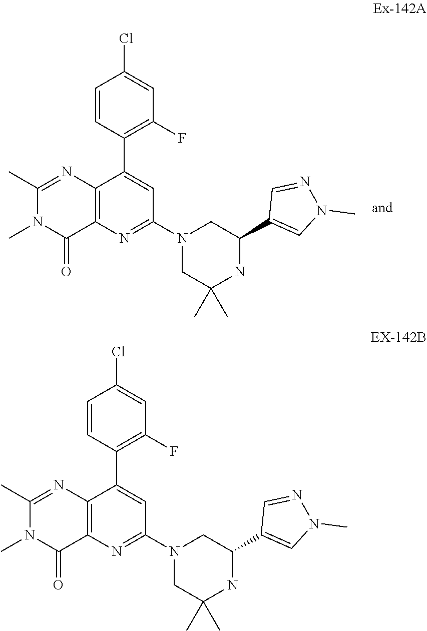

Chiral separation of 8-(4-chloro-2-fluorophenyl)-6-[2,2-dimethyl-6-(1-methyl-1H-pyrazol-4-yl)morpholin-4-yl]-2,3-dimethyl-3H,4H-pyrido[3,2-d]pyrimidin-4-one by normal phase chiral prep HPLC afforded (R) 8-(4-chloro-2-fluorophenyl)-6-[2,2-dimethyl-6-(1-methyl-1H-pyrazol-4-yl)morpholin-4-yl]-2,3-dimethyl-3H,4H-pyrido[3,2-d]pyrimidin-4-one [arbitrarily chosen Example 142A—Peak1] as off-white solid eluted first (S) 8-(4-chloro-2-fluorophenyl)-6-[2,2-dimethyl-6-(1-methyl-1H-pyrazol-4-yl)morpholin-4-yl]-2,3-dimethyl-3H,4H-pyrido[3,2-d]pyrimidin-4-one [arbitrarily chosen Example 142B—Peak2] as off-white solid eluted latter.

Example 142A, Peak1: 1H NMR (400 MHz, DMSO-d6) δ 7.70 (s, 1H), 7.60-7.38 (m, 5H), 4.79 (dd, J=2.7, 11.1 Hz, 1H), 4.58 (d, J=12.7 Hz, 1H), 4.32 (d, J=12.8 Hz, 1H), 3.80 (s, 3H), 3.50 (s, 3H), 2.87-2.76 (m, 2H), 2.40 (s, 3H), 1.25 (d, J=11.6 Hz, 6H).

Condition K: Rt=2.83 min. m/z 497.3 [M+H]⁺

Example 142B, Peak2: 1H NMR (400 MHz, DMSO-d6) δ 7.70 (s, 1H), 7.60-7.38 (m, 5H), 4.79 (dd, J=2.7, 11.1 Hz, 1H), 4.58 (d, J=12.7 Hz, 1H), 4.32 (d, J=12.8 Hz, 1H), 3.80 (s, 3H), 3.50 (s, 3H), 2.87-2.76 (m, 2H), 2.40 (s, 3H), 1.25 (d, J=11.6 Hz, 6H).

Condition K: Rt=2.83 min. m/z 497.3 [M+H]⁺

Analytical chiral HPLC: CHIRALPAK IC (250×4.6) mm, 5μ, M.P.—Hexane/DCM/EtOH/IPAmine—60/20/20/0.1, Flow Rate—1.0 ml/min. Solubility: MeOH Rt=12.47 min for Peak 1 and Rt=14.35 min for Peak 2.

### Example 143 to Example 147

Example 143 to Example 147 were synthesized using similar procedure as Example 112. Chiral separation details of Example 143A-143B to 147A-147B are captured in following

| Compound No. | | Starting | Chiral separation |
|---|---|---|---|
| Structure | Name | material | specification |
| Ex. 143A (Peak 1) 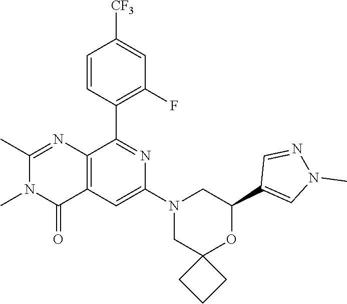 | (R)-8-(2-fluoro-4- (trifluoromethyl)phenyl)- 2,3-dimethyl-6-(6-(1- methyl-1H-pyrazol-4- yl)-5-oxa-8- azaspiro[3.5]nonan-8- yl)pyrido[3,4- d]pyrimidin-4(3H)-one | 8-(2-fluoro-4- (trifluoromethyl) phenyl)- 2,3-dimethyl- 6-(6-(1- methyl-1H- pyrazol-4-yl)- 5-oxa-8- azaspiro[3.5] nonan-8- yl)pyrido[3,4- d]pyrimidin- 4(3H)-one | Similar specifications as mentioned in step-6 of example 1 Peak 1 Absolute stereochemistry unknown |
| Ex. 143B (Peak 2) 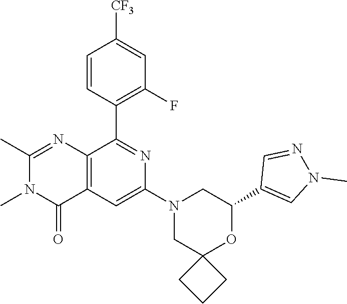 | (S)-8-(2-fluoro-4- (trifluoromethyl)phenyl)- 2,3-dimethyl-6-(6-(1- methyl-1H-pyrazol-4- yl)-5-oxa-8- azaspiro[3.5]nonan-8- yl)pyrido[3,4-d] pyrimidin-4(3H)-one | | Peak 2 Absolute stereochemistry unknown |
| Ex. 144A (Peak 1) 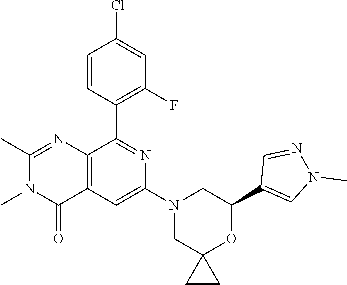 | (R)-8-(4-chloro-2- fluorophenyl)-2,3- dimethyl-6-(5-(1- methyl-1H-pyrazol-4- yl)-4-oxa-7- azaspiro[2.5]octan-7- yl)pyrido[3,4- d]pyrimidin-4(3H)-one | 8-(4-chloro- 2-fluorophenyl)- 2,3- dimethyl-6- (5-(1-methyl- 1H-pyrazol- 4-yl)-4-oxa-7- azaspiro[2.5] octan-7- yl)pyrido[3,4- d]pyrimidin- 4(3H)-one | Similar specifications as mentioned in step-6 of example 1 Peak 1 Absolute stereochemistry unknown |
| Ex. 144B (Peak 2) 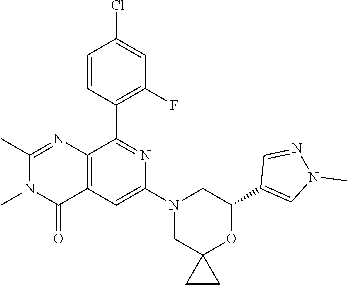 | (S)-8-(4-chloro-2- fluorophenyl)-2,3- dimethyl-6-(5-(1- methyl-1H-pyrazol-4- yl)-4-oxa-7- azaspiro[2.5]octan-7- yl)pyrido[3,4- d]pyrimidin-4(3H)-one | | Peak 2 Absolute stereochemistry unknown |
| Ex. 145A (Peak 1) 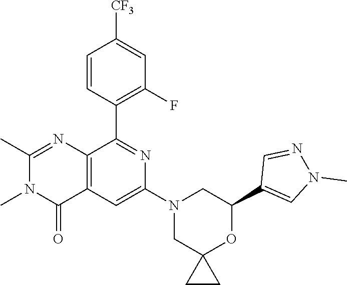 | (R)-8-(2-fluoro-4- (trifluoromethyl)phenyl)- 2,3-dimethyl-6-(5-(1- methyl-1H-pyrazol-4- yl)-4-oxa-7- azaspiro[2.5]octan-7- yl)pyrido[3,4- d]pyrimidin-4(3H)-one | 8-(2-fluoro-4- (trifluoromethyl) phenyl)-2,3- dimethyl-6-(5- (1-methyl-1H- pyrazol-4-yl)- 4-oxa-7- azaspiro[2.5] octan-7-yl) pyrido[3,4-d] pyrimidin- 4(3H)-one | Similar specifications as mentioned in step- 6 of example 1 Peak 1 Absolute stereochemistry unknown |
| Ex. 145B (Peak 2 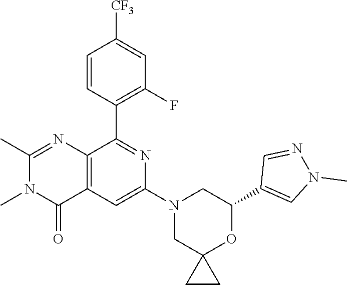 | (S)-8-(2-fluoro-4- (trifluoromethyl)phenyl)- 2,3-dimethyl-6-(5-(1- methyl-1H-pyrazol-4- yl)-4-oxa-7- azaspiro[2.5]octan-7- yl)pyrido[3,4- d]pyrimidin-4(3H)-one | | Peak 2 Absolute stereochemistry unknown |
| Ex. 146A (Peak 1) 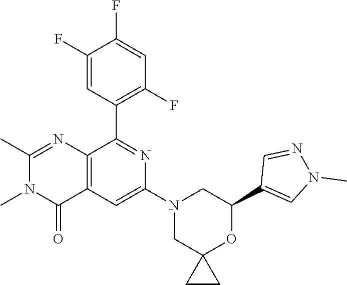 | (R)-2,3-dimethyl-6-(5- (1-methyl-1H-pyrazol- 4-yl)-4-oxa-7- azaspiro[2.5]octan-7- yl)-8-(2,4,5- trifluorophenyl)pyrido [3,4-d]pyrimidin- 4(3H)-one | 2,3-dimethyl- 6-(5-(1- methyl-1H- pyrazol-4-yl)- 4-oxa-7- azaspiro[2.5] octan-7-yl)-8- (2,4,5- trifluorophenyl) pyrido[3,4-d] pyrimidin- 4(3H)-one | Similar specifications as mentioned in step- 6 of example 1 Peak 1 Absolute stereochemistry unknown |
| Ex. 146B (Peak 2) 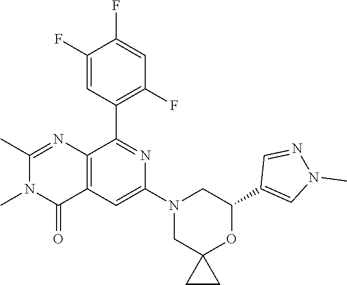 | (S)-2,3-dimethyl-6-(5- (1-methyl-1H-pyrazol- 4-yl)-4-oxa-7- azaspiro[2.5]octan-7- yl)-8-(2,4,5- trifluorophenyl)pyrido [3,4-d]pyrimidin- 4(3H)-one | | Peak 2 Absolute stereochemistry unknown |
| Ex. 147A (Peak 1) 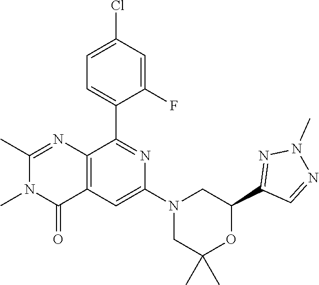 [SMILES: Cc1nc2c(-c3ccc(Cl)cc3F)nc(N3C[C@@H](c4cnn(C)n4)OC(C)(C)C3)cc2c(=O)n1C] | (S)-8-(4-chloro-2- fluorophenyl)-6-(2,2- dimethyl-6-(2-methyl- 2H-1,2,3-triazol-4- yl)morpholino)-2,3- dimethylpyrido[3,4- d]pyrimidin-4(3H)-one | 8-(4-chloro-2- fluorophenyl)-6- (2,2- dimethyl-6- (2-methyl- 2H-1,2,3- triazol-4- yl)morpholino)- 2,3-dimethyl- pyrido[3,4-d] pyrimidin- 4(3H)-one | Similar specifications as mentioned in step- 6 of example 1 Peak 1 Absolute stereochemistry unknown |
| Ex. 147B (Peak 2) 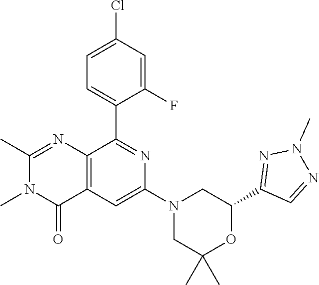 [SMILES: Cc1nc2c(-c3ccc(Cl)cc3F)nc(N3C[C@H](c4cnn(C)n4)OC(C)(C)C3)cc2c(=O)n1C] | (R)-8-(4-chloro-2- fluorophenyl)-6-(2,2- dimethyl-6-(2-methyl- 2H-1,2,3-triazol-4- yl)morpholino)-2,3- dimethylpyrido[3,4- d]pyrimidin-4(3H)-one | | Peak 2 Absolute stereochemistry unknown |

Following table describes analytical data analysis and yield information of examples 143A-143B to examples 147A-147B:

| Compbound | | | Analytical | | Yield |
|---|---|---|---|---|---|
| No. | HNMR | LCMS | chiral HPLC | Description | (%) |
| Ex. | ¹H-NMR (400 MHZ, DMSO-d6): δ | Condition | CHIRALPAK IG | Yellow | 43.9 |
| 143A | 7.80-7.77 (m, 2 H), 7.73-7.69 (m, | K: Rt = | (250 × 4.6) mm, | solid | |
| (Peak 1) | 2 H), 7.45 (s, 1 H), 7.41 (s, 1 H), | 3.11 min. | 5 μ M.P.- | | |
| | 4.56 (d, J = 10.8 Hz, 1 H), 4.38 (d, | m/z 543.3 | Hexane/DCM/ | | |
| | J = 12.4 Hz, 1 H), 4.27 (d, J = 12.0 | [M + H]⁺. | EtOH/IPAmine- | | |
| | Hz, 1 H), 3.80 (s, 3 H), 3.31 (s, | | 50/25/25/0.1 | | |
| | 3 H), 2.86-2.78 (m, 2 H), 2.43 (s, | | Flow Rate-1.0 | | |
| | 3 H), 2.03-1.96 (m, 4 H), 1.79- | | ml/min. | | |
| | 1.77 (m, 2 H). | | Solubility: | | |
| Ex. | ¹H-NMR (400 MHZ, DMSO-d6): δ | Condition | MeOH Rt = 4.79 | Yellow | 44.2 |
| 143B | 7.80-7.77 (m, 2 H), 7.73-7.69 (m, | K: Rt = | min for Peak 1 | solid | |
| (Peak 2) | 2 H), 7.45 (s, 1 H), 7.41 (s, 1 H), | 3.11 min. | and Rt = 5.52 | | |
| | 4.56 (d, J = 10.8 Hz, 1 H), 4.38 (d, | m/z 543.3 | min for Peak 2. | | |
| | J = 12.4 Hz, 1 H), 4.27 (d, J = 12.0 | [M + H]⁺. | | | |
| | Hz, 1 H), 3.80 (s, 3 H), 3.31 (s, | | | | |
| | 3 H), 2.86-2.78 (m, 2 H), 2.43 (s, | | | | |
| | 3 H), 2.03-1.96 (m, 4 H), 1.79- | | | | |
| | 1.77 (m, 2 H). | | | | |
| Ex. | ¹H-NMR (400 MHZ, DMSO-d6): δ | Condition | CHIRALPAK IG | Yellow | 42.1 |
| 144A | 7.72 (s, 1 H), 7.57-7.50 (m, | K: Rt = | (250 × 4.6) mm, | solid | |
| (Peak 1) | 2 H), 7.44 (s, 1 H), 7.40-7.37 (m, | 3.01 min. | 5 μ M.P.- | | |
| | 1 H), 7.30 (s, 1 H), 4.72-4.68 (dd, | m/z 495.3 | Hexane/DCM/ | | |
| | J = 10.8 Hz, J = 2.0 Hz, 1 H), 4.39 | [M + H]⁺. | EtOH/IPAmine- | | |
| | (d, J = 12.0 Hz, 1 H), 3.79 (s, 3 H), | | 50/25/25/0.1 | | |
| | 3.68 (d, J = 11.6 Hz, 1 H), 3.51 (s, | | Flow Rate-1.0 | | |
| | 3 H), 3.47-3.44 (m, 1 H), 2.98 (t, | | ml/min. | | |
| | J = 11.2 Hz, 1 H), 2.43 (s, 3 H), | | Solubility: | | |
| | 0.86-0.79 ( m, 2 H), 0.70-0.64 (m, | | MeOH Rt = 6.62 | | |
| | 2 H). | | min for Peak 1 | | |
| Ex. | ¹H-NMR (400 MHZ, DMSO-d6): δ | Condition | and Rt = 10.97 | Yellow | 41.6 |
| 144B | 7.72 (s, 1 H), 7.57-7.50 (m, | K: Rt = | min for Peak 2. | solid | |
| (Peak 2) | 2 H), 7.44 (s, 1 H), 7.40-7.37 (m, | 3.01 min. | | | |
| | 1 H), 7.30 (s, 1 H), 4.72-4.68 (dd, | m/z 495.3 | | | |
| | J = 10.8 Hz, J = 2.0 Hz, 1 H), 4.39 | [M + H]⁺. | | | |
| | (d, J = 12.0 Hz, 1 H), 3.79 (s, 3 H), | | | | |
| | 3.68 (d, J = 11.6 Hz, 1 H), 3.51 (s, | | | | |
| | 3 H), 3.47-3.44 (m, 1 H), 2.98 (t, | | | | |
| | J = 11.2 Hz, 1 H), 2.43 (s, 3 H), | | | | |
| | 0.86-0.79 (m, 2 H), 0.70-0.64 (m, | | | | |
| | 2 H). | | | | |
| Ex. | ¹H-NMR (400 MHZ, DMSO-d6): δ | Condition | CHIRALPAK IG | Yellow | 38.5 |
| 145A | 7.79-7.68 (m, 4 H), 7.44 (s, 1 H), | K: Rt = | (250 × 4.6) mm, | Solid | |
| (Peak 1) | 7.35 (s, 1 H), 4.69 (d, J = 8.8 Hz, | 3.08 min. | 5 μ M.P.- | | |
| | 1 H), 4.39 (d, J = 12.4 Hz, 1 H), | m/z 529.4 | Hexane/DCM/ | | |
| | 3.79 (s, 3 H), 3.69 (d, J = 12.4 Hz, | [M + H]⁺. | EtOH/IPAmine- | | |
| | 1 H), 3.51 (s, 3 H), 3.44 (d, J = 12.0 | | 60/20/20/0.1 | | |
| | Hz, 1 H), 3.02-2.96 (m, 1 H), 2.98 | | Flow Rate-1.0 | | |
| | (t, J = 11.2 Hz, 1 H), 2.43 (s, 3 H), | | ml/min. | | |
| | 0.86-0.80 ( m, 2 H), 0.72-0.63 (m, | | MeOH Rt = 6.81 | | |
| | 2 H). | | min for Peak 1 | | |
| Ex. | ¹H-NMR (400 MHZ, DMSO-d6): δ | Condition | and Rt = 9.64 | Yellow | 37.6 |
| 145B | 7.79-7.68 (m, 4 H), 7.44 (s, 1 H), | K: Rt = | min for Peak 2. | Solid | |
| (Peak 2) | 7.35 (s, 1 H), 4.69 (d, J = 8.8 Hz, | 3.08 min. | | | |
| | 1 H), 4.39 (d, J = 12.4 Hz, 1 H), | m/z 529.4 | | | |
| | 3.79 (s, 3 H), 3.69 (d, J = 12.4 Hz, | [M + H]⁺. | | | |
| | 1 H), 3.51 (s, 3 H), 3.44 (d, J = 12.0 | | | | |
| | Hz, 1 H), 3.02-2.96 (m, 1 H), 2.98 | | | | |
| | (t, J = 11.2 Hz, 1 H), 2.43 (s, 3 H), | | | | |
| | 0.86-0.80 ( m, 2 H), 0.72-0.63 (m, | | | | |
| | 2 H). | | | | |
| Ex. | ¹H-NMR (400 MHZ, DMSO-d6): δ | Condition | CHIRALPAK IG | Yellow | 40.2 |
| 146A | 7.72 (s, 1 H), 7.68-7.60 (m, 2 H), | K: Rt = | (250 × 4.6) mm, | Solid | |
| (Peak 1) | 7.44 (s, 1 H), 7.32 (s, 1 H), 4.69 | 2.89 min. | 5 μ M.P.- | | |
| | (d, J = 9.2 Hz, 1 H), 4.39 (d, J = | m/z 497.3 | Hexane/DCM/ | | |
| | 12.0 Hz, 1 H), 3.79 (s, 3 H), 3.68 | [M + H]⁺. | EtOH/IPAmine- | | |
| | (d, J = 12.4 Hz, 1 H), 3.51 (s, 3 H), | | 50/25/25/0.1 | | |
| | 3.47 (d, J = 12.8 Hz, 1 H), 3.02- | | Flow Rate-1.0 | | |
| | 2.95 (m, 1 H), 2.43 (s, 3 H), 0.82- | | ml/min | | |
| | 0.80 ( m, 2 H), 0.72-0.64 (m, 2 H). | | solubility | | |
| Ex. | ¹H-NMR (400 MHZ, DMSO-d6): δ | Condition | MeOH Rt = 5.84 | Yellow | 39.8 |
| 146B | 7.72 (s, 1 H), 7.68-7.60 (m, 2 H), | K: Rt = | min for Peak 1 | Solid | |
| (Peak 2) | 7.44 (s, 1 H), 7.32 (s, 1 H), 4.69 | 2.89 min. | and Rt = 9.98 | | |
| | (d, J = 9.2 Hz, 1 H), 4.39 (d, J = | m/z 497.3 | min for Peak 2. | | |
| | 12.0 Hz, 1 H), 3.79 (s, 3 H), 3.68 | [M + H]⁺. | | | |
| | (d, J = 12.4 Hz, 1 H), 3.51 (s, 3 H), | | | | |
| | 3.47 (d, J = 12.8 Hz, 1 H), 3.02- | | | | |
| | 2.95 (m, 1 H), 2.43 (s, 3 H), 0.82- | | | | |
| | 0.80 ( m, 2 H), 0.72-0.64 (m, 2 H). | | | | |
| Ex. | ¹H-NMR (400 MHZ, DMSO-d6): δ | Condition | Waters Thar | Yellow | 20.0 |
| 147A | 7.73 (s, 1 H), 7.58-7.51 (m, 2 H), | P: Rt = | SFC-80 | Solid | |
| (Peak 1) | 7.41-7.38 (dd, J = 8.0 Hz, J = 2.0 | 3.05 min. | equipped with | | |
| | Hz, 1 H), 7.32 (s, 1 H), 5.02-4.99 | m/z 498.2 | Waters UV | | |
| | (dd, J = 11.2 Hz, J = 2.8 Hz, 1 H), | [M + H]⁺. | Detector 2489 | | |
| | 4.49 (d, J = 11.6 Hz, 1 H), 4.18- | | by using | | |
| | 4.15 (m, 1 H), 4.13 (s, 3 H), 3.51 | | Chiralpak-IG | | |
| | (s, 3 H), 2.99-2.93 (m, 1 H), 2.86 | | (30.0 mm × | | |
| | (d, J = 12.8 Hz, 1 H), 2.43 (s, 3 H), | | 250 mm), 5 μ | | |
| | 1.32 (s, 3 H), 1.26 (s, 3 H). | | Column | | |
| Ex. | ¹H-NMR (400 MHZ, DMSO-d6): δ | Condition | operating at 35 | Yellow | 17.2 |
| 147B | 7.73 (s, 1 H), 7.58-7.51 (m, 2 H), | P: Rt = | ° C. temperature, | Solid | |
| (Peak 2) | 7.41-7.38 (dd, J = 8.0 Hz, J = 2.0 | 3.05 min. | maintaining flow | | |
| | Hz, 1 H), 7.32 (s, 1 H), 5.02-4.99 | m/z 498.2 | rate of 60 ml/min | | |
| | (dd, J = 11.2 Hz, J = 2.8 Hz, 1 H), | [M + H]⁺. | using 75% CO2 | | |
| | 4.49 (d, J = 11.6 Hz, 1 H), 4.18- | | in super critical | | |
| | 4.15 (m, 1 H), 4.13 (s, 3 H), 3.51 | | state & 25% of | | |
| | (s, 3 H), 2.99-2.93 (m, 1 H), 2.86 | | (ACN:MEOH | | |
| | (d, J = 12.8 Hz, 1 H), 2.43 (s, 3 H), | | (1:1)) as Mobile | | |
| | 1.32 (s, 3 H), 1.26 (s, 3 H). | | phase | | |
| | | | solubility | | |
| | | | MeOH Rt = 3.23 | | |
| | | | min for Peak 1 | | |
| | | | and Rt = 9.68 | | |
| | | | min for Peak 2. | | |

### Example 148

### Example 148A—Peak1

### 8-(4-chloro-2-fluorophenyl)-2,3-dimethyl-6-((1R,2S)-2-(1-methyl-1H-pyrazol-4-yl)cyclopropyl)pyrido[3,4-d]pyrimidin-4 (3H)-one

### Example 148B—Peak2

### 8-(4-chloro-2-fluorophenyl)-2,3-dimethyl-6-((1R,2R)-2-(1-methyl-1H-pyrazol-4-yl)cyclopropyl)pyrido[3,4-d]pyrimidin-4 (3H)-one

### Step-1—Preparation of 4-ethenyl-1-methyl-1H-pyrazole

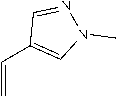

To a stirred solution of 4-bromo-1-methyl-1H-pyrazole (2.5 g, 15.52 mmol) and potassium-vinyltrifluoroborate (6.24 g, 46.56 mmol) in dioxane (40 mL) and water (1 mL) was added cesium carbonate (10.2 g, 31.06 mmol) and degassed with argon. Pd(dppf)Cl₂·DCM (1.27 g, 1.56 mmol) was added under inert atmosphere. The resulting mixture was heated at 100° C. for 12 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by (50-60% ethyl acetate in hexane to afford 4-ethenyl-1-methyl-1H-pyrazole (300 mg, 18% yield) as colourless oil.

¹H-NMR (400 MHz, DMSO-d6): δ 7.76 (s, 1H), 7.55 (s, 1H), 6.53-6.41 (m, 1H), 5.41 (d, J=17.6 Hz, 1H), 5.03-4.96 (m, 1H), 3.78 (s, 3H).

### Step-2—Preparation of 1-methyl-4-[2-(4,4,5,5-tetramethyl-1,3,2-dioxaborolan-2-yl)cyclopropyl]-1H-pyrazole

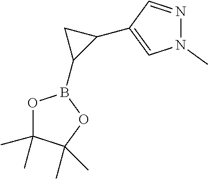

To a stirred solution of CrCl₂ (3.4 g, 27.74 mmol) and TMEDA (4.16 mL, 27.74 mmol) in dry THF (20 mL) at RT for 1 h and (diiodomethyl)-4,4,5,5-tetramethyl-1,3,2-dioxaborolane (1.3 g, 3.3 mmol) was added. Resulting mixture was stirred for 1 h at RT. 1-cyclopropyl-4-(2,5-dihydrofuran-2-yl)-1H-pyrazole (0.3 g, 2.8 mmol) was added. Reaction mixture was heated to 50° C. for 20 h. Reaction mixture was diluted with water and ethyl acetate. Combined mixture was filtered through a short pad of celite. Filtrate was washed with brine, dried over sodium sulphate and concentrated into vacuo.

Crude was purified by column chromatography (10-30% ethyl acetate in hexane) to afford 1-methyl-4-[2-(4,4,5,5-tetramethyl-1,3,2-dioxaborolan-2-yl)cyclopropyl]-1H-pyrazole (450.0 mg; 65.4% yield) as colourless oil.

LCMS: Condition G: Rt=3.30 min. m/z 249.0 [M+H]⁺.

### Step-3—Preparation of 8-(4-chloro-2-fluorophenyl)-2,3-dimethyl-6-((1R,2S)-2-(1-methyl-1H-pyrazol-4-yl)cyclopropyl)pyrido[3,4-d]pyrimidin-4 (3H)-one (Example 148A) and 8-(4-chloro-2-fluorophenyl)-2,3-dimethyl-6-((1R,2R)-2-(1-methyl-1H-pyrazol-4-yl)cyclopropyl)pyrido[3,4-d]pyrimidin-4 (3H)-one (Example 148B)

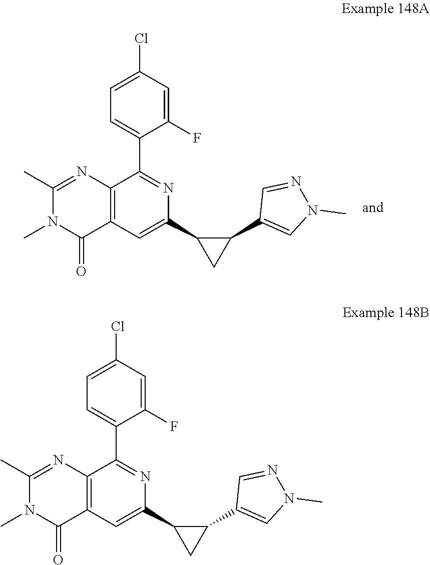

To a stirred solution of 6-chloro-8-(4-chloro-2-fluorophenyl)-2,3-dimethylpyrido[3,4-d]pyrimidin-4 (3H)-one (200.0 mg, 0.6 mmol) and 1-methyl-4-[2-(4,4,5,5-tetramethyl-1,3,2-dioxaborolan-2-yl)cyclopropyl]-1H-pyrazole (220.0 mg, 0.9 mmol) in toluene (8 mL) and water (2 mL) was added cesium carbonate (384.2 mg, 1.2 mmol) and degassed with argon. CataCXiumA (63.42 mg, 0.18 mmol) and Pd(OAc)₂ (26.5 mg, 0.118 mmol) were added under inert atmosphere. The resulting mixture was heated at 100° C. for 8 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by reverse-phase prep HPLC to obtain 8-(4-chloro-2-fluorophenyl)-2,3-dimethyl-6-((1R,2S)-2-(1-methyl-1H-pyrazol-4-yl)cyclopropyl)pyrido[3,4-d]pyrimidin-4 (3H)-one (Example 148A, Peak1, 28.0 mg, 11.2%) as white solid (racemic mixture) and 8-(4-chloro-2-fluorophenyl)-2,3-dimethyl-6-((1R,2R)-2-(1-methyl-1H-pyrazol-4-yl)cyclopropyl)pyrido[3,4-d]pyrimidin-4 (3H)-one (Example 148B, Peak2, 30.0 mg, 12.0%) as white solid (racemic mixture). Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 55% A and 45% B in 3 min, then to 25% A and 75% B in 20 min., then to 5% A and 95% B in 21 min., held this composition up to 22 min. for column washing, then returned to initial composition in 23 min. and held till 25 min.

### Example 148A

¹H-NMR (400 MHz, DMSO-d6): δ 7.79 (s, 1H), 7.52 (d, J=10.8 Hz, 1H), 7.40 (d, J=8.0 Hz, 1H), 7.27 (s, 1H), 7.22 (t, J=8.0 Hz, 1H), 6.94 (s, 1H), 3.59 (s, 3H), 3.49 (s, 3H), 2.77-2.71 (m, 1H), 2.44 (s, 3H), 2.37-2.31 (m, 1H), 1.68-1.64 (m, 1H), 1.46-1.41 (m, 1H).

LCMS: Condition K: Rt=2.79 min. m/z 424.2 [M+H]⁺.

### Example 148B

¹H-NMR (400 MHz, DMSO-d6): δ 7.79 (s, 1H), 7.52 (d, J=10.8 Hz, 1H), 7.40 (d, J=8.0 Hz, 1H), 7.27 (s, 1H), 7.22 (t, J=8.0 Hz, 1H), 6.94 (s, 1H), 3.59 (s, 3H), 3.49 (s, 3H), 2.77-2.71 (m, 1H), 2.44 (s, 3H), 2.37-2.31 (m, 1H), 1.68-1.64 (m, 1H), 1.46-1.41 (m, 1H).

LCMS: Condition K: Rt=2.79 min. m/z 424.2 [M+H]⁺.

### Example 149 to Example 152

Example 149 to Example 152 were synthesized using similar procedure as Example 1 and 112.

Chiral separation details of Example 149A-149B to 152A-152B are captured in following

| | | | Chiral |
|---|---|---|---|
| Compound No. | | | separation |
| Structure | Name | Starting material | specification |
| Ex. 149A (Peak 1) | (R)-8-(4-chloro-2- | 8-(4-chloro-2- | Similar |
| | fluorophenyl)-6-(2,2- | fluorophenyl)-6- | specifications |
| 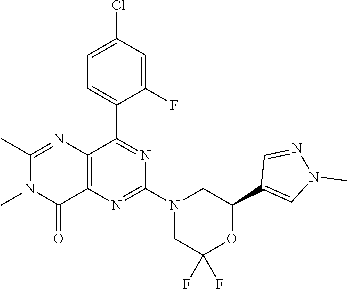 | difluoro-6-(1-methyl-1H- pyrazol-4-yl)morpholino)- 2,3-dimethylpyrimido[5,4- d]pyrimidin-4(3H)-one | (2,2-difluoro-6-(1- methyl-1H- pyrazol-4- yl)morpholino)- 2,3- dimethylpyrimido [5,4-d]pyrimidin- 4(3H)-one (Example-115) | as mentioned in step-6 of example 1 Peak 1 Absolute stereo- chemistry unknown |
| Ex. 149B (Peak 2) | (S)-8-(4-chloro-2- | | Peak 2 |
| | fluorophenyl)-6-(2,2- | | Absolute |
| 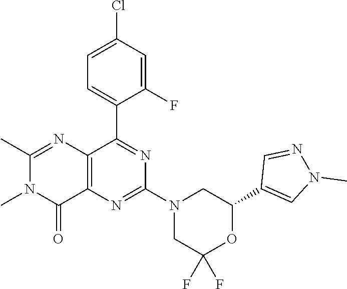 | difluoro-6-(1-methyl-1H- pyrazol-4-yl)morpholino)- 2,3-dimethylpyrimido[5,4- d]pyrimidin-4(3H)-one | | stereo- chemistry unknown |
| Ex. 150A (Peak 1) | (R)-6-(2,2-dimethyl-6-(1- | 6-(2,2-dimethyl-6- | Similar |
| | methyl-1H-pyrazol-4- | (1-methyl-1H- | specifications |
| 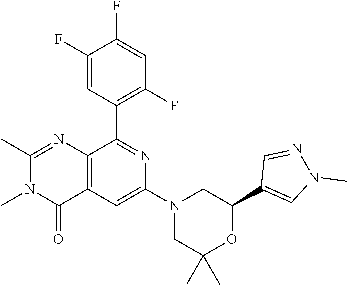 | yl)morpholino)-2,3- dimethyl-8-(2,4,5- trifluorophenyl)pyrido [3,4- d]pyrimidin-4(3H)-one | pyrazol-4- yl)morpholino)- 2,3-dimethyl-8- (2,4,5- trifluorophenyl) pyrido[3,4- d]pyrimidin-4(3H)- one | as mentioned in step-6 of example 1 Peak 1 Absolute stereo- chemistry unknown |
| Ex. 150B (Peak 2) | (S)-6-(2,2-dimethyl-6-(1- | | Peak 2 |
| | methyl-1H-pyrazol-4- | | Absolute |
| 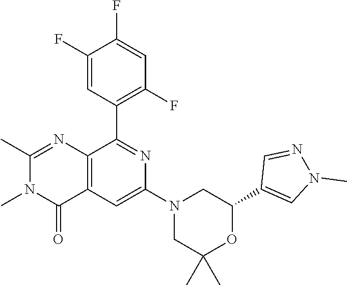 | yl)morpholino)-2,3- dimethyl-8-(2,4,5- trifluorophenyl)pyrido[3,4- d]pyrimidin-4(3H)-one | | stereo- chemistry unknown |
| Ex. 151A (Peak 1) | (R)-8-(4-chloro-2- | 8-(4-chloro-2- | Similar |
| | fluorophenyl)-2,3- | fluorophenyl)-2,3- | specifications |
| 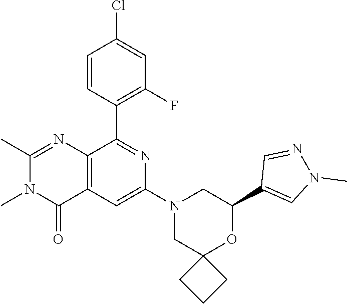 | dimethyl-6-(6-(1-methyl- 1H-pyrazol-4-yl)-5-oxa-8- azaspiro[3.5]nonan-8- yl)pyrido[3,4-d]pyrimidin- 4(3H)-one | dimethyl-6-(6-(1- methyl-1H- pyrazol-4-yl)-5- oxa-8- azaspiro[3.5] nonan-8-yl) pyrido[3,4- d]pyrimidin-4(3H)- one | as mentioned in step-6 of example 1 Peak 1 Absolute stereo- chemistry unknown |
| Ex. 151B (Peak 2) | (S)-8-(4-chloro-2- | | Peak 2 |
| | fluorophenyl)-2,3- | | Absolute |
| 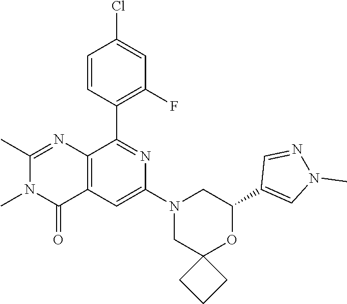 | dimethyl-6-(6-(1-methyl- 1H-pyrazol-4-yl)-5-oxa-8- azaspiro[3.5]nonan-8- yl)pyrido[3,4-d]pyrimidin- 4(3H)-one | | stereo- chemistry unknown |
| Ex. 152A (Peak 1) | (R)-2,3-dimethyl-6-(6-(1- | 2,3-dimethyl-6-(6- | Similar |
| | methyl-1H-pyrazol-4-yl)-5- | (1-methyl-1H- | specifications |
| 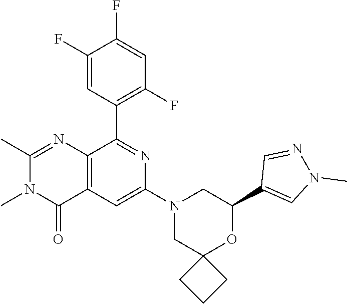 | oxa-8-azaspiro[3.5]nonan- 8-yl)-8-(2,4,5- trifluorophenyl)pyrido[3,4- d]pyrimidin-4(3H)-one | pyrazol-4-yl)-5- oxa-8- azaspiro[3.5]nonan- 8-yl)-8-(2,4,5- trifluorophenyl) pyrido[3,4- d]pyrimidin-4(3H)- one | as mentioned in step-6 of example 1 Peak 1 Absolute stereo- chemistry unknown |
| Ex. 152B (Peak 2) | (S)-2,3-dimethyl-6-(6-(1- | | Peak 2 |
| | methyl-1H-pyrazol-4-yl)-5- | | Absolute |
| 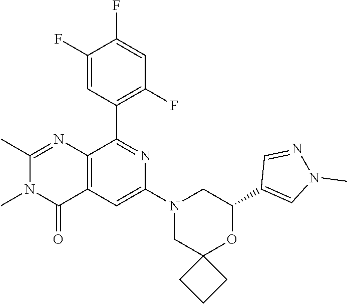 | oxa-8-azaspiro[3.5]nonan- 8-yl)-8-(2,4,5- trifluorophenyl)pyrido[3,4- d]pyrimidin-4(3H)-one | | stereo- chemistry unknown |

Following table describes analytical data analysis and yield information of examples 149A-149B to examples 152A-152B:

| | | | Analytical | | Yield |
|---|---|---|---|---|---|
| Compound No. | HNMR | LCMS | chiral HPLC | Description | (%) |
| Ex. | ¹H-NMR (400 MHZ, DMSO- | Condition K: | Chiralpak IG | Yellow | 35.1 |
| 149A | d6): δ 7.92 (s, 1 H), 7.70 (t, | Rt = 2.99 | (4.6 × 250 mm), | solid | |
| (Peak 1) | J = 8.0 Hz, 1 H), 7.63-7.59 (m, | min. m/z | 5 μ | | |
| | 2 H), 7.46 ( d, J = 6.8 Hz, 1 H), | 506.3 | ARD/K/ | | |
| | 5.29-5.26 (dd, J = 11.2 Hz, | [M + H]⁺. | Mobile Phase: | | |
| | J = 2.4 Hz, 1 H), 4.94 (d, J = | | Hexane/DCM/ | | |
| | 12.4 Hz, 1 H), 4.70 (d, J = 12.8 | | EtOH/IPAmine: | | |
| | Hz, 1 H), 3.84 (s, 3 H), 3.77- | | 50/25/25/0.1 | | |
| | 3.67 (m, 2 H), 3.54 (s, 3 H), | | Flow Rate: 1.0 | | |
| | 2.45 (s, 3 H). | | ml/min | | |
| Ex. | ¹H-NMR (400 MHZ, DMSO- | Condition K: | Solubility: | Yellow | 35.9 |
| 149B | d6): δ 7.92 (s, 1 H), 7.70 (t, | Rt = 2.99 | MeOH | solid | |
| (Peak 2) | J = 8.0 Hz, 1 H), 7.63-7.59 (m, | min. m/z | Rt = 6.88 min for | | |
| | 2 H), 7.46 ( d, J = 6.8 Hz, 1 H), | 506.3 | Peak 1 and Rt = | | |
| | 5.29-5.26 (dd, J = 11.2 Hz, | [M + H]⁺. | 8.02 min for | | |
| | J = 2.4 Hz, 1 H), 4.94 (d, J = | | Peak 2. | | |
| | 12.4 Hz, 1 H), 4.70 (d, J = 12.8 | | | | |
| | Hz, 1 H), 3.84 (s, 3 H), 3.77- | | | | |
| | 3.67 (m, 2 H), 3.54 (s, 3 H), | | | | |
| | 2.45 (s, 3 H). | | | | |
| Ex. | ¹H-NMR (400 MHZ, DMSO- | Condition K: | CHIRALPAK IG | Yellow | 42.1 |
| 150A | d6): δ 7.70 (s, 1 H), 7.64-7.62 | Rt = 2.92 | (250 × 4.6) mm, | solid | |
| (Peak 1) | (m, 2 H), 7.43 (s, 1 H), 7.33 (s, | min. m/z | 5 μ M.P.- | | |
| | 1 H), 4.81 (d, J = 9.6 Hz, 1 H), | 499.3 | Hexane/DCM/ | | |
| | 4.36 (d, J = 11.6 Hz, 1 H), 4.12 | [M + H]⁺. | EtOH/IPAmine- | | |
| | (d, J = 12.8 Hz, 1 H), 3.79 (s, | | 50/25/25/0.1 | | |
| | 3 H), 3.51 (s, 3 H), 2.81-2.75 | | Flow Rate-1.0 | | |
| | (m, 2 H), 2.32 (s, 3 H), 1.29 (s, | | ml/minMeOH | | |
| | 3 H), 1.07 (s, 3 H). | | Rt = 5.57 min for | | |
| Ex. | ¹H-NMR (400 MHZ, DMSO- | Condition K: | Peak 1 and Rt = | Yellow | 41.6 |
| 150B | d6): δ 7.70 (s, 1 H), 7.64-7.62 | Rt = 2.92 | 8.20 min for | solid | |
| (Peak 2) | (m, 2 H), 7.43 (s, 1 H), 7.33 (s, | min. m/z | Peak 2. | | |
| | 1 H), 4.81 (d, J = 9.6 Hz, 1 H), | 499.3 | | | |
| | 4.36 (d, J = 11.6 Hz, 1 H), 4.12 | [M + H]⁺. | | | |
| | (d, J = 12.8 Hz, 1 H), 3.79 (s, | | | | |
| | 3 H), 3.51 (s, 3 H), 2.81-2.75 | | | | |
| | (m, 2 H), 2.32 (s, 3 H), 1.29 (s, | | | | |
| | 3 H), 1.07 (s, 3 H). | | | | |
| Ex. | ¹H-NMR (400 MHZ, DMSO- | Condition K: | CHIRALPAK IG | Yellow | 39.5 |
| 151A | d6): 7.73 (s, 1 H), 7.59-7.51 | Rt = 3.04 | (250 × 4.6) mm, | Solid | |
| (Peak 1) | (m, 2 H), 7.45 (s, 1 H), 7.40- | min. m/z | 5 μ M.P.- | | |
| | 7.38 (m, 1 H), 7.36 (s, 1 H), | 509.3 | Hexane/DCM/ | | |
| | 4.55 (d, J = 10.8 Hz, 1 H), 4.37 | [M + H]⁺. | EtOH/IPAmine- | | |
| | (d, J = 12.8 Hz, 1 H), 4.26 (d, | | 50/25/25/0.1 | | |
| | J = 12.4 Hz, 1 H), 3.80 (s, 3 H), | | Flow Rate-1.0 | | |
| | 3.51 (s, 3 H), 2.85-2.77 (m, | | ml/min. | | |
| | 2 H), 2.43 (s, 3 H), 2.08-1.99 | | solubility: | | |
| | (m, 4 H), 1.79-1.75 (m, 2 H). | | MeOH, Rt = | | |
| Ex. | ¹H-NMR (400 MHZ, DMSO- | Condition K: | 6.56 min for | Yellow | 38.6 |
| 151B | d6): 7.73 (s, 1 H), 7.59-7.51 | Rt = 3.04 | Peak 1 and Rt = | Solid | |
| (Peak 2) | (m, 2 H), 7.45 (s, 1 H), 7.40- | min. m/z | 8.71 min for | | |
| | 7.38 (m, 1 H), 7.36 (s, 1 H), | 509.3 | Peak 2. | | |
| | 4.55 (d, J = 10.8 Hz, 1 H), 4.37 | [M + H]⁺. | | | |
| | (d, J = 12.8 Hz, 1 H), 4.26 (d, | | | | |
| | J = 12.4 Hz, 1 H), 3.80 (s, 3 H), | | | | |
| | 3.51 (s, 3 H), 2.85-2.77 (m, | | | | |
| | 2 H), 2.43 (s, 3 H), 2.08-1.99 | | | | |
| | (m, 4 H), 1.79-1.75 (m, 2 H). | | | | |
| Ex. | ¹H-NMR (400 MHZ, DMSO- | Condition K: | Chiralpak IG | Yellow | 40.2 |
| 152A | d6): 7.73 (s, 1 H), 7.67-7.63 | Rt = 2.95 | (4.6 × 250 mm), | Solid | |
| (Peak 1) | (m, 2 H), 7.45 (s, 1 H), 7.38 | min. m/z | 5 μ | | |
| | (s, 1 H), 4.56 (d, J = 8.8 Hz, | 511.4 | ARD/K/ | | |
| | 1 H), 4.37 (d, J = 12.4 Hz, 1 H), | [M + H]⁺. | Mobile Phase: | | |
| | 4.27 (d, J = 12.0 Hz, 1 H), 3.80 | | Hexane/DCM/ | | |
| | (s, 3 H), 3.52 (s, 3 H), 2.85- | | EtOH/IPAmine: | | |
| | 2.77 (m, 2 H), 2.44 (s, 3 H), | | 50/25/25/0.1 | | |
| | 2.08-1.99 (m, 4 H), 1.80-1.75 | | Flow Rate: 1.0 | | |
| | (m, 2 H). | | ml/min | | |
| Ex. | ¹H-NMR (400 MHZ, DMSO- | Condition K: | Solubility: | Yellow | 39.8 |
| 152B | d6): 7.73 (s, 1 H), 7.67-7.63 | Rt = 2.95 | MeOH | Solid | |
| (Peak 2) | (m, 2 H), 7.45 (s, 1 H), 7.38 | min. m/z | Rt = 6.08 min for | | |
| | (s, 1 H), 4.56 (d, J = 8.8 Hz, | 511.4 | Peak 1 and Rt = | | |
| | 1 H), 4.37 (d, J = 12.4 Hz, 1 H), | [M + H]⁺. | 8.09 min for | | |
| | 4.27 (d, J = 12.0 Hz, 1 H), 3.80 | | Peak 2. | | |
| | (s, 3 H), 3.52 (s, 3 H), 2.85- | | | | |
| | 2.77 (m, 2 H), 2.44 (s, 3 H), | | | | |
| | 2.08-1.99 (m, 4 H), 1.80-1.75 | | | | |
| | (m, 2 H). | | | | |

### Example 153A—Peak1

### 6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one

### Example 153B—Peak2

### 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one

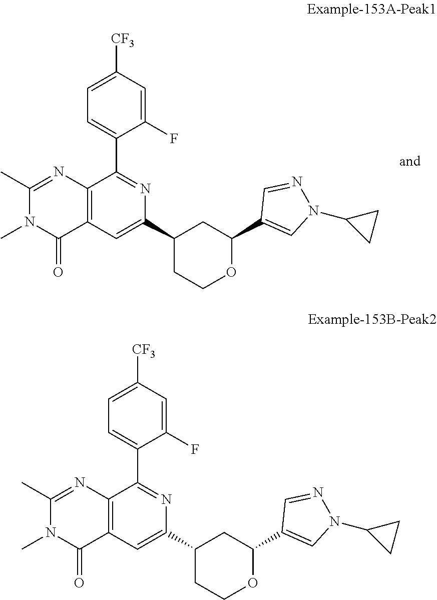 [SMILES: Cc1nc2c(-c3ccc(C(F)(F)F)cc3F)nc([C@@H]3CCO[C@H](c4cnn(C5CC5)c4)C3)cc2c(=O)n1C ; Cc1nc2c(-c3ccc(C(F)(F)F)cc3F)nc([C@H]3CCO[C@@H](c4cnn(C5CC5)c4)C3)cc2c(=O)n1C]

The racemic compound 6-(2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one (60 mg, 59%, white solid) was synthesized according to similar procedure depicted in Example 17.

Chiral separation of 6-(2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one (60 mg) was done by normal phase chiral prep HPLC to afford Example 153A, 6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one

(28.1 mg, 42%; eluted first, assigned as Peak 1 with arbitrary assignment of stereochemistry) as white solid and Example 153B, 6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one (29.0 mg, 43% yield, eluted later, assigned as Peak 2 with arbitrary assignment of stereochemistry) as white solid.

Normal phase Chiral prep HPLC method details to separate the enantiomers: Instrument: Agilent 1200 series instrument

Column: CHIRALPAK IG (250×21 mm) 5u/Flow rate: 21.0 ml/min./Mobile phase: Hexane/Dichloromethane/Ethanol: 60/20/20/Solubility: Mixture of Acetonitrile and Dichloromethane/Wave length: 306 nm/Run time: 20 min

Example 153A—Peak1: 1H NMR (400 MHz, DMSO-d6) δ 7.94 (s, 1H), 7.83-7.78 (m, 2H), 7.74-7.71 (m, 2H), 4.45 (d, J=10.4 Hz, 1H), 4.08-4.05 (m, 1H), 3.66-3.63 (m, 2H), 3.55 (s, 3H), 2.55 (s, 3H), 2.17-2.13 (m, 1H), 1.89-1.80 (m, 3H), 0.98-0.96 (m, 2H), 0.92-0.89 (m, 2H).

LCMS Condition K: Rt=2.95 min. m/z 528.3 [M+H]⁺.

Example 153B—Peak2: 1H NMR (400 MHz, DMSO-d6) δ 7.94 (s, 1H), 7.83-7.78 (m, 2H), 7.74-7.71 (m, 2H), 4.45 (d, J=10.4 Hz, 1H), 4.08-4.05 (m, 1H), 3.66-3.63 (m, 2H), 3.55 (s, 3H), 2.55 (s, 3H), 2.17-2.13 (m, 1H), 1.89-1.80 (m, 3H), 0.98-0.96 (m, 2H), 0.92-0.89 (m, 2H).

LCMS Condition K: Rt=2.95 min. m/z 528.3 [M+H]⁺.

Analytical chiral HPLC: Waters Chiralcel OX-H, Mobile Phase: 0.3% IPAmine in MeOH, Flow Rate: 1.0 ml/m. Solubility: MeOH, Rt=3.78 m for Peak 1 and Rt=4.62 min for Peak 2.

### Example 154 to Example 156

Example 154 to Example 156 were synthesized using similar procedure as Example 1 and 103.

Chiral separation details of Example 154A-154B to 156A-156B are captured in following table:

| | | | Chiral |
|---|---|---|---|
| Compound No. | | | separation |
| Structure | Name | Starting material | specification |
| Ex. 154A (Peak 1) | (R)-8-(2,4-difluorophenyl)- | 8-(2,4- | Similar |
| | 3-methyl-6-(2-(2- | difluorophenyl)-3- | specifications |
| 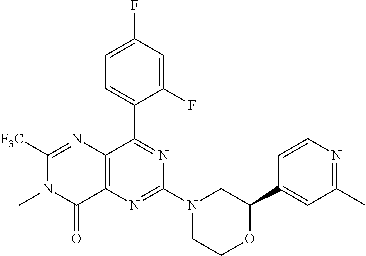 | methylpyridin-4- yl)morpholino)-2- (trifluoromethyl)pyrimido [5,4-d]pyrimidin- 4(3H)-one | methyl-6-(2-(2- methylpyridin-4- yl)morpholino)-2- (trifluoromethyl) pyrimido[5,4- d]pyrimidin-4(3H)- one | as mentioned in step-6 of example 1 Peak 1 Absolute stereo- chemistry unknown |
| Ex. 154B (Peak 2) | (S)-8-(2,4-difluorophenyl)- | | Peak 2 |
| | 3-methyl-6-(2-(2- | | Absolute |
| 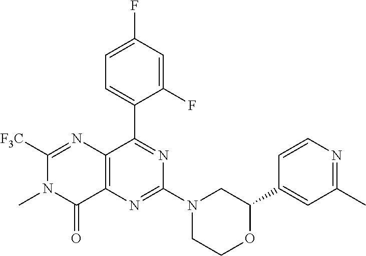 | methylpyridin-4- yl)morpholino)-2- (trifluoromethyl)pyrimido [5,4-d]pyrimidin- 4(3H)-one | | stereo- chemistry unknown |
| Ex. 155A (Peak 1) | (R)-3-methyl-6-(2-(2- | 3-methyl-6-(2-(2- | Similar |
| | methylpyridin-4- | methylpyridin-4- | specifications |
| 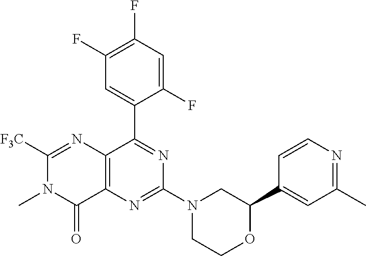 | yl)morpholino)-2- (trifluoromethyl)-8-(2,4,5- trifluorophenyl)pyrimido[5, 4-d]pyrimidin-4(3H)-one | yl)morpholino)-2- (trifluoromethyl)- 8-(2,4,5- trifluorophenyl) pyrimido[5,4- d]pyrimidin-4(3H)- one | as mentioned in step-6 of example 1 Peak 1 Absolute stereo- chemistry unknown |
| Ex. 155B (Peak 2) | (S)-3-methyl-6-(2-(2- | | Peak 2 |
| | methylpyridin-4- | | Absolute |
| 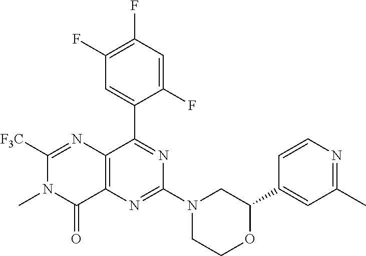 | yl)morpholino)-2- (trifluoromethyl)-8-(2,4,5- trifluorophenyl)pyrimido[5, 4-d]pyrimidin-4(3H)-one | | stereo- chemistry unknown |
| Ex. 156A (Peak 1) | (R)-6-(2,2-difluoro-6-(1- | 6-(2,2-difluoro-6-(1- | Similar |
| | methyl-1H-pyrazol-4- | methyl-1H-pyrazol- | specifications |
| 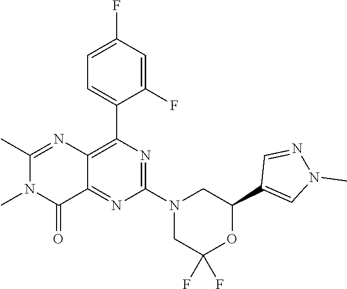 | yl)morpholino)-8-(2,4- difluorophenyl)-2,3- dimethylpyrimido [5,4- d]pyrimidin-4(3H)-one | 4-yl)morpholino)-8- (2,4- difluorophenyl)-2,3- dimethylpyrimido[5, 4-d]pyrimidin- 4(3H)-one | as mentioned in step-6 of example 1 Peak 1 Absolute stereo- chemistry unknown |
| Ex. 156B (Peak 2) | (S)-6-(2,2-difluoro-6-(1- | | Peak 2 |
| | methyl-1H-pyrazol-4- | | Absolute |
| 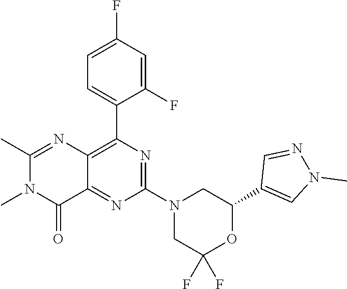 | yl)morpholino)-8-(2,4- difluorophenyl)-2,3- dimethylpyrimido [5,4- d]pyrimidin-4(3H)-one | | stereo- chemistry unknown |

Following table describes analytical data analysis and yield information of examples 154A-154B to examples 156A-156B:

| | | | Analytical | | Yield |
|---|---|---|---|---|---|
| Compound No. | HNMR | LCMS | chiral HPLC | Description | (%) |
| Ex. 154A | ¹H-NMR (400 MHZ, CDCI₃): δ | Condition | Chiralpak IG | Yellow | 35.1 |
| (Peak 1) | 8.51 (d, J = 5.2 Hz, 1 H), 7.72- | K: Rt = 2.64 | (4.6 × 250 mm), | solid | |
| | 7.66 (m, 1 H), 7.45-7.37 (m, | min. m/z | 5 μ | | |
| | 2 H), 7.05-6.99 (m, 2 H), 5.04 | 519.3 | ARD/K/ | | |
| | (s, 1 H), 4.88 (s, 1 H), 4.57 (d, | [M + H]⁺. | Mobile Phase: | | |
| | J = 5.4 Hz, 1 H), 4.22-4.18 (m, | | Hexane/DCM/ | | |
| | 1 H), 3.84-3.78(m, 1 H), 3.73 | | EtOH/IPAmine: | | |
| | (s, 3 H), 3.34 (t, J = 11.2 Hz, | | 50/25/25/0.1 | | |
| | 1 H), 3.10-3.04 (m, 1 H), 2.68 | | Flow Rate: 1.0 | | |
| | (s, 3 H). | | ml/min | | |
| Ex. 154B | ¹H-NMR (400 MHZ, CDCI₃): δ | Condition | Solubility: | Yellow | 35.9 |
| (Peak 2) | 8.51 (d, J = 5.2 Hz, 1 H), 7.72- | K: Rt = 2.64 | MeOH | solid | |
| | 7.66 (m, 1 H), 7.45-7.37 (m, | min. m/z | Rt = 4.86 min | | |
| | 2 H), 7.05-6.99 (m, 2 H), 5.04 | 519.3 | for Peak 1 and | | |
| | (s, 1 H), 4.88 (s, 1 H), 4.57 (d, | [M + H]⁺. | Rt = 6.31 min | | |
| | J = 5.4 Hz, 1 H), 4.22-4.18 (m, | | for Peak 2. | | |
| | 1 H), 3.84-3.78(m, 1 H), 3.73 | | | | |
| | (s, 3 H), 3.34 (t, J = 11.2 Hz, | | | | |
| | 1 H), 3.10-3.04 (m, 1 H), 2.68 | | | | |
| | (s, 3 H). | | | | |
| Ex. 155A | ¹H-NMR (400 MHZ, CDCI₃): δ | Condition | CHIRALPAK IG | Yellow | 42.1 |
| (Peak 1) | 8.55 (d, J = 5.6 Hz, 1 H), 7.58- | K: Rt = 2.26 | (250 × 4.6) mm, | solid | |
| | 7.50 (m, 3 H), 7.12-7.05 (m, | min. m/z | 5 μ M.P.- | | |
| | 1 H), 5.06 (s, 1 H), 4.88 (s, 1 H), | 537.3 | Hexane/DCM/ | | |
| | 4.63 (d, J = 8.8 Hz, 1 H), 4.24- | [M + H]⁺. | EtOH/IPAmine- | | |
| | 4.20 (m, 1 H), 3.86-3.80(m, | | 50/25/25/0.1 | | |
| | 1 H), 3.73 (s, 3 H), 3.38-3.32 | | Flow Rate-1.0 | | |
| | (m, 1 H), 3.10-3.04 (m, 1 H), | | ml/minMeOH | | |
| | 2.79 (s, 3 H). | | Rt = 4.69 min | | |
| Ex. 155B | ¹H-NMR (400 MHZ, CDCI₃): δ | Condition | for Peak 1 and | Yellow | 41.6 |
| (Peak 2) | 8.55 (d, J = 5.6 Hz, 1 H), 7.58- | K: Rt = 2.26 | Rt = 5.87 min | solid | |
| | 7.50 (m, 3 H), 7.12-7.05 (m, | min. m/z | for Peak 2. | | |
| | 1 H), 5.06 (s, 1 H), 4.88 (s, 1 H), | 537.3 | | | |
| | 4.63 (d, J = 8.8 Hz, 1 H), 4.24- | [M + H]⁺. | | | |
| | 4.20 (m, 1 H), 3.86-3.80(m, | | | | |
| | 1 H), 3.73 (s, 3 H), 3.38-3.32 | | | | |
| | (m, 1 H), 3.10-3.04 (m, 1 H), | | | | |
| | 2.79 (s, 3 H). | | | | |
| Ex. 156A | ¹H-NMR (400 MHZ, DMSO- | Condition | CHIRALPAK | Yellow | 39.5 |
| (Peak 1) | d6): δ 7.92 (s, 1 H), 7.75-7.73 | K: Rt = 2.80 | IG | Solid | |
| | (m, 1 H), 7.59 (s, 1 H), 7.42- | min. m/z | (250 × 4.6) mm, | | |
| | 7.40 (m, 1 H), 7.26 (t, J = 1.6 | 490.3 | 5 μ M.P.- | | |
| | Hz, 1 H), 5.29-5.26 (dd, J = | [M + H]⁺. | Hexane/DCM/ | | |
| | 12.0 Hz, J = 2.4 Hz, 1 H), 4.94 | | EtOH/IPAmine- | | |
| | (d, J = 12.8 Hz, 1 H), 4.71 (d, | | 50/25/25/0.1 | | |
| | J = 13.2 Hz, 1 H), 3.84 (s, 3 H), | | Flow Rate-1.0 | | |
| | 3.77-3.67 (m, 2 H), 3.52 (s, | | ml/min. | | |
| | 3 H), 2.46 (s, 3 H). | | solubility: | | |
| Ex. 156B | ¹H-NMR (400 MHZ, DMSO- | Condition | MeOH, Rt = | Yellow | 38.6 |
| (Peak 2) | d6): δ 7.92 (s, 1 H), 7.75-7.73 | K: Rt = 2.80 | 7.20 min for | Solid | |
| | (m, 1 H), 7.59 (s, 1 H), 7.42- | min. m/z | Peak 1 and Rt = | | |
| | 7.40 (m, 1 H), 7.26 (t, J = 1.6 | 490.3 | 8.75 min for | | |
| | Hz, 1 H), 5.29-5.26 (dd, J = | [M + H]⁺. | Peak 2. | | |
| | 12.0 Hz, J = 2.4 Hz, 1 H), 4.94 | | | | |
| | (d, J = 12.8 Hz, 1 H), 4.71 (d, | | | | |
| | J = 13.2 Hz, 1 H), 3.84 (s, 3 H), | | | | |
| | 3.77-3.67 (m, 2 H), 3.52 (s, | | | | |
| | 3 H), 2.46 (s, 3 H). | | | | |

### Example 157

### 8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one

### Step-1—Preparation of tert-butyl (6-chloro-5-fluoropyridin-3-yl)carbamate

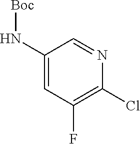 [SMILES: CC(C)(C)OC(=O)Nc1cnc(Cl)c(F)c1]

To a stirred solution of 5-bromo-2-chloro-3-fluoropyridine (5.0 g, 23.94 mmol) and tert-butyl carbamate (3.08 g, 26.33 mmol) in dioxane (50 mL), cesium carbonate (15.50 g, 47.87 mmol) was added and degassed with argon. Pd₂(dba)₃ (1.095 g, 1.19 mmol) was added under inert atmosphere. Resulting mixture was heated at 80° C. for 16 h. The reaction mixture was diluted with ethyl acetate, filtered through a pad of celite and washed with ethyl acetate. The combined organic layers were washed with water, brine, dried over anhydrous sodium sulphate, filtered and concentrated under reduced pressure. The crude product was purified by column chromatography (70% ethyl acetate in hexane) to afford tert-butyl (6-chloro-5-fluoropyridin-3-yl)carbamate (3.9 g, 67% yield) as pale yellow solid.

LCMS Condition N: Rt=2.11 min. m/z 247.05 [M+H]⁺.

### Step-2—Preparation of 5-((tert-butoxycarbonyl)amino)-2-chloro-3-fluoroisonicotinic acid

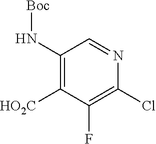 [SMILES: CC(C)(C)OC(=O)Nc1cnc(Cl)c(F)c1C(=O)O]

To a stirred solution of tert-butyl (6-chloro-5-fluoropyridin-3-yl)carbamate (2.0 g, 8.13 mmol) in diethyl ether (50 mL) at −78° C. under argon atmosphere, n-BuLi (1.6 M in hexane, 16.0 mL, 24.4 mmol) was added dropwise and stirred for 0.5 h. Then temperature of reaction mixture was increased to −20° C. and stirred for another 0.5 h. Reaction mixture was again cooled to −78° C. and purged with CO₂ gas for 1 h. Reaction mixture was quenched with aqueous ammonium chloride and acidified with dilute HCl and extracted with ethyl acetate. Combined organic layer was washed with brine, dried over anhydrous sodium sulphate, filtered and concentrated under reduced pressure to afford 5-((tert-butoxycarbonyl)amino)-2-chloro-3-fluoroisonicotinic acid (1.9 g, 80% yield) as white solid.

LCMS Condition N: Rt=1.84 min. m/z 291.2 [M+H]⁺.

### Step-3—Preparation of tert-butyl (6-chloro-5-fluoro-4-(methylcarbamoyl)pyridin-3-yl) carbamate

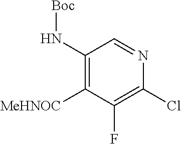

To a stirred solution of 5-(tert-butoxycarbonyl)amino)-2-chloro-3-fluoroisonicotinic acid (1.9 g, 6.55 mmol) in DMF (20 mL) at 0° C. was subsequently added MeNH₂·HCl (0.53 g, 7.86 mmol) followed by HATU (2.98 g, 7.86 mmol) and DIPEA (4.0 mL, 22.92 mmol). Resulting mixture was stirred at RT for 16 h. After completion, reaction mixture was poured into crushed ice. The aqueous layer was extracted with ethyl acetate, washed with water, brine, dried over anhydrous sodium sulphate, filtered and concentrated under reduced pressure. The crude product was purified by column chromatography 10% ethyl acetate in hexane) to afford tert-butyl (6-chloro-5-fluoro-4-(methylcarbamoyl)pyridin-3-yl)carbamate (1.4 g, 72% yield) as white solid.

LCMS Condition N: Rt=2.12 min. m/z 304.2 [M+H]⁺.

### Step-4—Preparation of 5-amino-2-chloro-3-fluoro-N-methylisonicotinamide

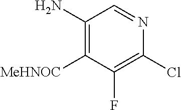

To a stirred solution of tert-butyl (6-chloro-5-fluoro-4-(methylcarbamoyl)pyridin-3-yl)carbamate (1.4 g, 4.6 mmol) in DCM (10 mL) was added TFA (10.0 mL, excess eq.) at 0° C. The resulting mixture was stirred at RT for 2 h. Reaction mixture was concentrated under reduced pressure. The crude product was neutralized with aqueous sodium bicarbonate solution and extracted with ethyl acetate and dried over anhydrous sodium sulphate, filtered and concentrated to afford 5-amino-2-chloro-3-fluoro-N-methylisonicotinamide (850 mg, 90% yield) as white solid.

LCMS Condition N: Rt=1.50 min. m/z 204.2 [M+H]⁺.

### Step-5—Preparation of 3-amino-2-bromo-6-chloro-5-fluoro-N-methylisonicotinamide

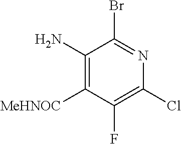

To a stirred solution of 5-amino-2-chloro-3-fluoro-N-methylisonicotinamide (850.0 mg 4.1 mmol) in DMF (5.0 mL) was added NBS (740 mg, 4.1 mmol) portion wise. The resulting mixture was heated at 80° C. for 1 h. After completion, reaction mixture was poured into crushed ice. The aqueous layer was extracted with ethyl acetate, washed with water, brine, dried over anhydrous sodium sulphate, filtered and concentrated under reduced pressure. The crude product was purified by column chromatography 30% ethyl acetate in hexane) to afford 3-amino-2-bromo-6-chloro-5-fluoro-N-methylisonicotinamide (1.0 g, 85% yield) as yellow solid.

LCMS Condition N: Rt=1.81 min. m/z 282.1 [M+H]⁺.

### Step-6—Preparation of 8-bromo-6-chloro-5-fluoro-2,3-dimethylpyrido[3,4-d]pyrimidin-4 (3H)-one

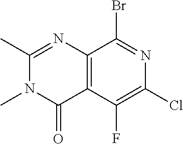 [SMILES: Cc1nc2c(Br)nc(Cl)c(F)c2c(=O)n1C]

To a stirred solution of 3-amino-2-bromo-6-chloro-5-fluoro-N-methylisonicotinamide (1.0 g, 3.55 mmol) in triethyl orthoacetate (6.5 mL, excess eq.) was added PTSA (1.02 g, 5.34 mmol). The resulting mixture was heated at 140° C. for 4 h. After completion, reaction mixture was neutralised with aqueous sodium bicarbonate solution and extracted with ethyl acetate, dried over anhydrous sodium sulphate, filtered and concentrated. The crude product was triturated with diethyl ether to afford 8-bromo-6-chloro-5-fluoro-2,3-dimethylpyrido[3,4-d]pyrimidin-4 (3H)-one (730 mg, 67% yield) as dark brown solid.

LCMS Condition N: Rt=2.29 min. m/z 306.1 [M+H]⁺

### Step-7—Preparation of 6-chloro-8-(4-chloro-2-fluorophenyl)-5-fluoro-2,3-dimethylpyrido[3,4-d]pyrimidin-4 (3H)-one

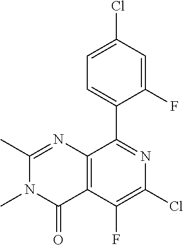 [SMILES: Cc1nc2c(-c3ccc(Cl)cc3F)nc(Cl)c(F)c2c(=O)n1C]

To a stirred solution of 8-bromo-6-chloro-5-fluoro-2,3-dimethylpyrido[3,4-d]pyrimidin-4 (3H)-one (750.0 mg 2.46 mmol) and (4-chloro-2-fluorophenyl)boronic acid (385.18 mg 2.21 mmol) in dioxane (30 mL) and water (5 mL) was added sodium carbonate (390.0 mg, 3.69 mmol) and degassed with argon. PdCl₂(dppf) (90.0 mg, 0.12 mmol) was added under inert atmosphere. The resulting mixture was heated at 60° C. for 1 h. Reaction mixture was diluted with ethyl acetate, filtered through a pad of celite and washed with ethyl acetate. Combined organic part was washed with water, brine, dried over anhydrous sodium sulphate, filtered and concentrated under reduced pressure. Crude product was purified by column chromatography (35% ethyl acetate-hexane) to afford 8-bromo-6-chloro-5-fluoro-2,3-dimethylpyrido[3,4-d]pyrimidin-4 (3H)-one (660 mg, 57% yield) as white solid.

LCMS Condition N: Rt=2.72 min. m/z 356.1 [M+H]⁺

### Step-8—Preparation of 8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one (Example-157)

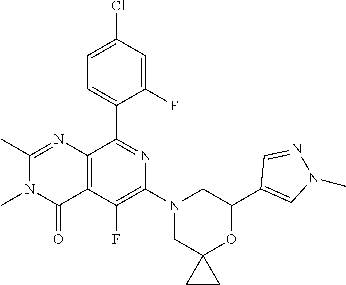

To a stirred solution of 8-bromo-6-chloro-5-fluoro-2,3-dimethylpyrido[3,4-d]pyrimidin-4 (3H)-one (200.0 mg 0.56 mmol) in toluene (30.0 mL) were added 5-(1-methyl-1H-pyrazol-4-yl)-4-oxa-7-azaspiro[2.5]octane (141.44 mg, 0.73 mmol), sodium tert-butoxide (108.29 mg, 1.12 mmol) and Ruphos (26.29 mg, 0.06 mmol). The resulting mixture was degassed with argon. Ru-Phos-Pd-G3 (47.0 mg 0.06 mmol) was added under inert atmosphere. The resulting mixture was heated at 70° C. for 4 h. After completion, reaction mixture was passed through celite bed and washed with DCM and concentrated. Crude product was purified by reverse phase preparative HPLC to afford 8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one (10 mg, 3.1% yield) as yellow solid.

Prep-HPLC: Preparative HPLC was done on Waters auto purification instrument. Column name: Xbridge C18 (250×19 mm, 5μ) operating at ambient temperature and flow rate of 16 mL/min. Mobile phase: A=20 mM Ammonium Bicarbonate in water, B=Acetonitrile:MEOH (50:50); Gradient Profile: Mobile phase initial composition of 60% A and 40% B, then 40% A and 60% B in 3 min, then to 10% A and 90% B in 20 min., then to 5% A and 95% B in 21 min., held this composition up to 22 min. for column washing, then returned to initial composition in 23 min. and held till 25 min.

1H NMR (400 MHz, DMSO-d6) δ 7.69 (s, 1H), 7.58-7.51 (m, 2H), 7.40-7.38 (m, 2H), 4.81 (d, J=10.4 Hz, 1H), 3.99 (d, J=12.8 Hz, 1H), 3.78 (s, 3H), 3.58 (d, J=12.8 Hz, 1H), 3.46 (s, 3H), 3.36 (d, J=13.2 Hz, 1H), 3.10-3.07 (m, 1H), 2.43 (s, 3H), 0.82-0.72 (m, 4H).

LCMS Condition K: Rt=3.10 min. m/z 513.3 [M+H]⁺.

### Example 158

### 8-(2,4-difluorophenyl)-5-fluoro-3-methyl-6-(5-(1-methyl-1H-pyrazol-4-yl)-4-oxa-7-azaspiro[2.5]octan-7-yl)-2-(trifluoromethyl)pyrido[3,4-d]pyrimidin-4 (3H)-one

### Step-1—Preparation of 3-amino-6-chloro-2-(2,4-difluorophenyl)-5-fluoro-N-methylisonicotinamide

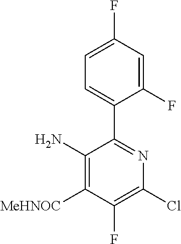

To a stirred solution of 3-amino-2-bromo-6-chloro-5-fluoro-N-methylisonicotinamide (1.0 g, 3.56 mmol, described in Example-157, step-5) and (2,4-difluorophenyl) boronic acid (506.0 mg 3.20 mmol) in dioxane (40 mL) and water (10 mL), K₃PO₄ (1.2 g, 5.339 mmol) was added and degassed with argon. Pd-118 (260.0 mg 0.35 mmol) was added to the reaction mixture under inert atmosphere. The resulting mixture was heated at 60° C. for 3 h. Reaction mixture was diluted with ethyl acetate, filtered through a pad of celite and washed with ethyl acetate. Combined organic part was washed with water, brine, dried over anhydrous sodium sulphate, filtered and concentrated under reduced pressure. Crude mass was purified by column chromatography (35% ethyl acetate in hexane) to afford 3-amino-6-chloro-2-(2,4-difluorophenyl)-5-fluoro-N-methylisonicotinamide (850 mg, 76% yield) as white solid.

LCMS Condition K: Rt=1.85 min. m/z 316.13 [M+H]⁺.

### Step-2—Preparation of 2-chloro-6-(2,4-difluorophenyl)-3-fluoro-N-methyl-5-(2,2,2-trifluoroacetamido)isonicotinamide

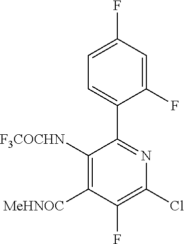

To a stirred solution of 3-amino-6-chloro-2-(2,4-difluorophenyl)-5-fluoro-N-methylisonicotinamide (850.0 mg, 2.69 mmol) in THF (10 mL) was added DMAP (66.0 mg 0.54 mmol) followed by pyridine (0.65 mL) at 0° C. Then added trifluoroacetic anhydride (0.6 mL, 8.1 mmol) dropwise at same temperature. Resulting mixture was allowed to stir at RT for 2 h. After completion, reaction mixture was poured into ice-cold water. The aqueous layer was extracted with ethyl acetate, washed with water, brine, dried over anhydrous sodium sulphate, filtered and concentrated under reduced pressure. The crude product was purified by column chromatography 10% ethyl acetate in hexane) to afford 2-chloro-6-(2,4-difluorophenyl)-3-fluoro-N-methyl-5-(2,2,2-trifluoroacetamido)isonicotinamide (750 mg, 65% yield) as gummy mass.

LCMS Condition K: Rt=1.90 min. m/z 412.1 [M+H]⁺.

### Step-3—Preparation of 6-chloro-8-(2,4-difluorophenyl)-5-fluoro-3-methyl-2-(trifluoromethyl)pyrido[3,4-d]pyrimidin-4 (3H)-one

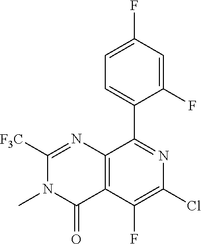 [SMILES: Cn1c(C(F)(F)F)nc2c(-c3ccc(F)cc3F)nc(Cl)c(F)c2c1=O]

To a stirred solution of 2-chloro-6-(2,4-difluorophenyl)-3-fluoro-N-methyl-5-(2,2,2-trifluoroacetamido)isonicotinamide (750.0 mg 1.83 mmol) in acetic acid (10 mL) was added sodium acetate (149.69 mg, 1.83 mmol). Resulting mixture was refluxed at 120° C. for 16 h. After completion, reaction mixture was evaporated to dryness. Crude product was quenched with aqueous sodium bicarbonate. The aqueous layer was extracted with ethyl acetate, washed with brine, dried over anhydrous sodium sulphate, filtered and concentrated under reduced pressure to afford 6-chloro-8-(2,4-difluorophenyl)-5-fluoro-3-methyl-2-(trifluoromethyl)pyrido[3,4-d]pyrimidin-4 (3H)-one (375 mg, 52% yield) as gum.

LCMS Condition K: Rt=2.84 min. m/z 394.22 [M+H]⁺.

### Step-4—Preparation of 8-(2,4-difluorophenyl)-5-fluoro-3-methyl-6-(5-(1-methyl-1H-pyrazol-4-yl)-4-oxa-7-azaspiro[2.5]octan-7-yl)-2-(trifluoromethyl)pyrido[3,4-d]pyrimidin-4 (3H)-one (Example-158)

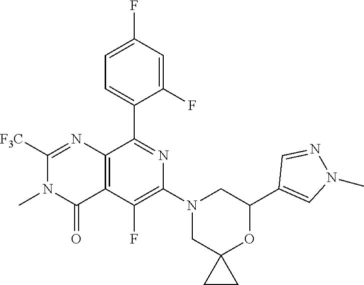

To a stirred solution of 6-chloro-8-(2,4-difluorophenyl)-5-fluoro-3-methyl-2-(trifluoromethyl)pyrido[3,4-d]pyrimidin-4 (3H)-one (100.0 mg, 0.25 mmol) in toluene (3.0 mL) were added subsequently 5-(1-methyl-1H-pyrazol-4-yl)-4-oxa-7-azaspiro[2.5]octane (98.0 mg, 0.50 mmol), sodium tert-butoxide (61.0 mg, 0.63 mmol) and Ruphos (12.0 mg, 0.03 mmol). The resulting mixture was degassed with argon RuPhos-Pd-G3 (21.0 mg, 0.03 mmol) was added under inert atmosphere. The resulting mixture was heated at 70° C. for 4 h. After completion, reaction mixture was passed through a pad of celite and washed with DCM and concentrated.

Crude product was purified by reverse phase preparative HPLC to afford 8-(2,4-difluorophenyl)-5-fluoro-3-methyl-6-(5-(1-methyl-1H-pyrazol-4-yl)-4-oxa-7-azaspiro[2.5]octan-7-yl)-2-(trifluoromethyl)pyrido[3,4-d]pyrimidin-4 (3H)-one (15.0 mg, 10% yield) as white solid.

Prep-HPLC: Preparative HPLC was done on Waters auto purification instrument. Column name: LONGYMC, C18 (20×250 mm), 5 μm operating at ambient temperature and flow rate of 16 mL/min. Mobile phase: A=Acetonitrile, B=20 mM Ammonium Bicarbonate; Gradient Profile: Mobile phase initial composition of 40% A and 60% B, then 65% A and 35% B in 3 min, then to 90% A and 10% B in 22 min., then to 100% A and 0% B in 22.5 min., held this composition up to 25 min. for column washing, then returned to initial composition in 25.5 min. and held till 28 min.

1H NMR (400 MHz, DMSO-d6) δ 7.71 (s, 1H), 7.64-7.7.62 (m, 1H), 7.42 (s, 1H), 7.37-7.36 (m, 1H), 7.23-7.22 (m, 1H), 4.81-4.78 (dd, J=10.4 Hz, J=2.4 Hz, 1H), 4.19 (d, J=13.2 Hz, 1H), 3.79 (s, 3H), 3.69 (d, J=13.3 Hz, 1H), 3.55 (d, J=13.3 Hz, 1H), 3.50 (s, 3H), 3.24-3.18 (m, 1H), 0.82-0.78 (m, 4H).

LCMS Condition K: Rt=9.12 min. m/z 551.5 [M+H]⁺.

### Example 159 to Example 166

Example 159 to Example 162 were synthesized using similar procedures as Example 1 and 112.

Chiral separation details of Example 159A-159B to 166A-166B are captured in following table:

| | | | Chiral |
|---|---|---|---|
| Compound No. | | | separation |
| Structure | Name | Starting material | specification |
| Ex. 159A (Peak 1) | (R)-8-(2,4- | 8-(2,4- | Similar |
| | difluorophenyl)-6-(2,2- | difluorophenyl)-6- | specifications as |
| 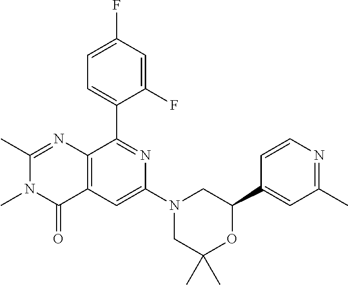 | dimethyl-6-(2- methylpyridin-4- yl)morpholino)-2,3- dimethylpyrido[3,4- d]pyrimidin-4(3H)-one | (2,2-dimethyl-6-(2- methylpyridin-4- yl)morpholino)-2,3- dimethylpyrido[3,4- d]pyrimidin-4(3H)- one | mentioned in step-6 of example 1 Peak 1 Absolute stereochemistry unknown |
| Ex. 159B (Peak 2) | (S)-8-(2,4- | | Peak 2 |
| | difluorophenyl)-6-(2,2- | | Absolute |
| 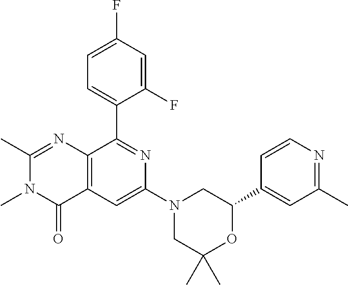 | dimethyl-6-(2- methylpyridin-4- yl)morpholino)-2,3- dimethylpyrido [3,4- d]pyrimidin-4(3H)-one | | stereochemistry unknown |
| Ex. 160A (Peak 1) | (R)-6-(2,2-dimethyl-6- | 6-(2,2-dimethyl-6-(2- | Similar |
| | (2-methylpyridin-4- | methylpyridin-4- | specifications as |
| 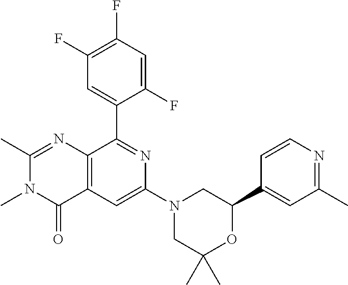 | yl)morpholino)-2,3- dimethyl-8-(2,4,5- trifluorophenyl)pyrido [3,4-d]pyrimidin- 4(3H)-one | yl)morpholino)-2,3- dimethyl-8-(2,4,5- trifluorophenyl)pyrido [3,4-d]pyrimidin- 4(3H)-one | mentioned in step-6 of example 1 Peak 1 Absolute stereochemistry unknown |
| Ex. 160B (Peak 2) | (S)-6-(2,2-dimethyl-6- | | Peak 2 |
| | (2-methylpyridin-4- | | Absolute |
| 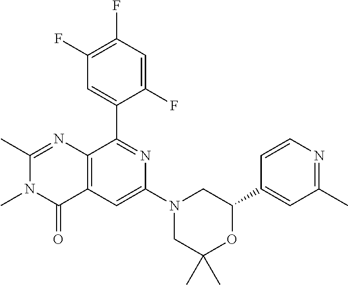 | yl)morpholino)-2,3- dimethyl-8-(2,4,5- trifluorophenyl)pyrido[3 ,4-d]pyrimidin-4(3H)- one | | stereochemistry unknown |
| Ex. 161A (Peak 1) | (R)-6-(2,2-dimethyl-6- | 6-(2,2-dimethyl-6-(2- | Similar |
| | (2-methylpyridin-4- | methylpyridin-4- | specifications as |
| 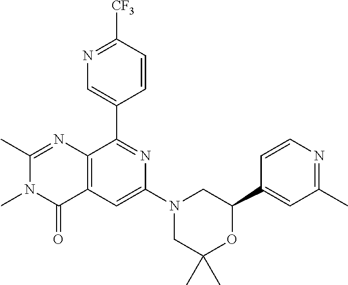 | yl)morpholino)-2,3- dimethyl-8-(6- (trifluoromethyl)pyridin- 3-yl)pyrido[3,4- d]pyrimidin-4(3H)-one | yl)morpholino)-2,3- dimethyl-8-(6- (trifluoromethyl) pyridin-3-yl)pyrido [3,4-d]pyrimidin- 4(3H)-one | mentioned in step-6 of example 1 Peak 1 Absolute stereochemistry unknown |
| Ex. 161B (Peak 2) | (S)-6-(2,2-dimethyl-6- | | Peak 2 |
| | (2-methylpyridin-4- | | Absolute |
| 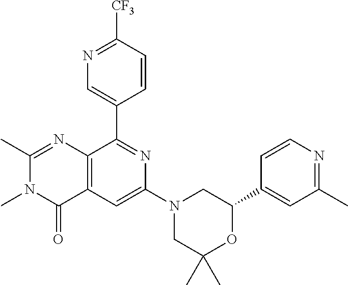 | yl)morpholino)-2,3- dimethyl-8-(6- (trifluoromethyl)pyridin- 3-yl)pyrido[3,4- d]pyrimidin-4(3H)-one | | stereochemistry unknown |
| Ex. 162A (Peak 1) | (R)-3-methyl-6-(5-(1- | 3-methyl-6-(5-(1- | Similar |
| | methyl-1H-pyrazol-4- | methyl-1H-pyrazol-4- | specifications as |
| 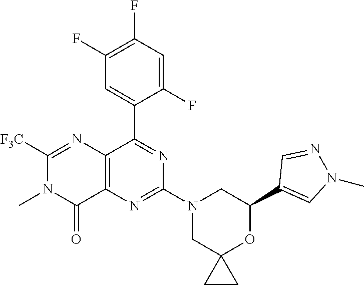 | yl)-4-oxa-7- azaspiro[2.5]octan-7- yl)-2-(trifluoromethyl)- 8-(2,4,5- trifluorophenyl) pyrimido[5,4-d] pyrimidin- 4(3H)-one | yl)-4-oxa-7- azaspiro[2.5]octan- 7-yl)-2- (trifluoromethyl)-8- (2,4,5- trifluorophenyl) pyrimido[5,4-d] pyrimidin-4(3H)-one | mentioned in step-6 of example 1 Peak 1 Absolute stereochemistry unknown |
| Ex. 162B (Peak 2) | (S)-3-methyl-6-(5-(1- | | Peak 2 |
| | methyl-1H-pyrazol-4- | | Absolute |
| 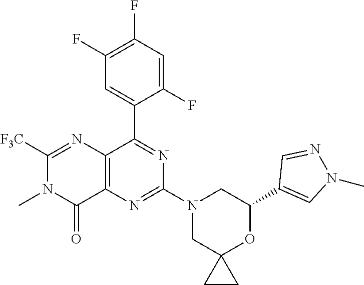 | yl)-4-oxa-7- azaspiro[2.5]octan-7- yl)-2-(trifluoromethyl)- 8-(2,4,5- trifluorophenyl)pyrimid o[5,4-d]pyrimidin- 4(3H)-one | | stereochemistry unknown |
| Ex. 163A (Peak 1) | (R)-6-(2,2-dimethyl-6- | 6-(2,2-dimethyl-6-(2- | Similar |
| | (2-methylpyridin-4- | methylpyridin-4- | specifications as |
| 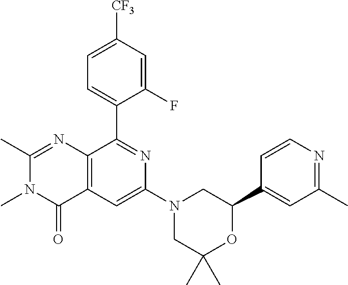 | yl)morpholino)-8-(2- fluoro-4- (trifluoromethyl) phenyl)-2,3- dimethylpyrido[3,4- d]pyrimidin-4(3H)-one | yl)morpholino)-8-(2- fluoro-4- (trifluoromethyl) phenyl)-2,3- dimethylpyrido[3,4- d]pyrimidin-4(3H)- one | mentioned in step-6 of example 1 Peak 1 Absolute stereochemistry unknown |
| Ex. 163B (Peak 2) | (S)-6-(2,2-dimethyl-6- | | Peak 2 |
| | (2-methylpyridin-4- | | Absolute |
| 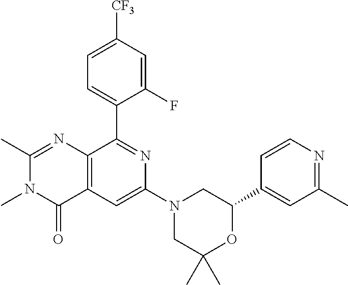 | yl)morpholino)-8-(2- fluoro-4- (trifluoromethyl) phenyl)-2,3- dimethylpyrido[3,4- d]pyrimidin-4(3H)-one | | stereochemistry unknown |
| Ex. 164A (Peak 1) | (R)-6-(2,2-dimethyl-6- | 6-(2,2-dimethyl-6-(2- | Similar |
| | (2-methylpyridin-4- | methylpyridin-4- | specifications as |
| 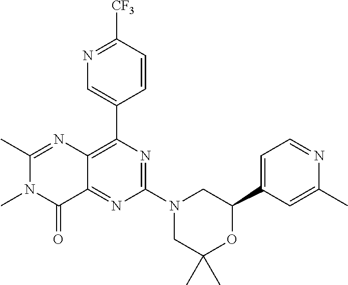 | yl)morpholino)-2,3- dimethyl-8-(6- (trifluoromethyl)pyridin- 3-yl)pyrimido[5,4- d]pyrimidin-4(3H)-one | yl)morpholino)-2,3- dimethyl-8-(6- (trifluoromethyl) pyridin-3-yl) pyrimido[5,4- d]pyrimidin-4(3H)- one | mentioned in step-6 of example 1 Peak 1 Absolute stereochemistry unknown |
| Ex. 164B (Peak 2) | (S)-6-(2,2-dimethyl-6- | | Peak 2 |
| | (2-methylpyridin-4- | | Absolute |
| 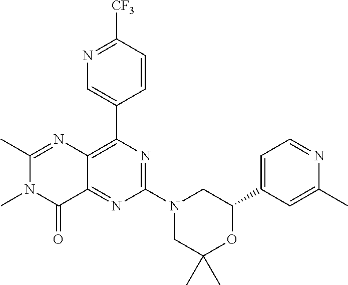 | yl)morpholino)-2,3- dimethyl-8-(6- (trifluoromethyl)pyridin- 3-yl)pyrimido[5,4- d]pyrimidin-4(3H)-one | | stereochemistry unknown |
| Ex. 165A (Peak 1) | (R)-8-(2,4- | 8-(2,4- | Similar |
| | difluorophenyl)-3- | difluorophenyl)-3- | specifications as |
| 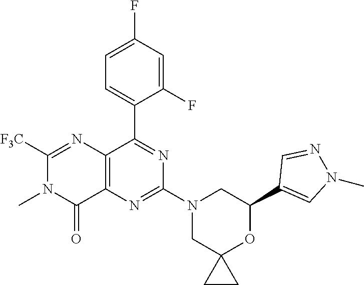 | methyl-6-(5-(1-methyl- 1H-pyrazol-4-yl)-4- oxa-7- azaspiro[2.5]octan-7- yl)-2- (trifluoromethyl)pyrimid o[5,4-d]pyrimidin- 4(3H)-one | methyl-6-(5-(1- methyl-1H-pyrazol-4- yl)-4-oxa-7- azaspiro[2.5]octan- 7-yl)-2- (trifluoromethyl) pyrimido[5,4-d] pyrimidin- 4(3H)-one | mentioned in step-6 of example 1 Peak 1 Absolute stereochemistry unknown |
| Ex. 165B (Peak 2) | (S)-8-(2,4- | | Peak 2 |
| | difluorophenyl)-3- | | Absolute |
| 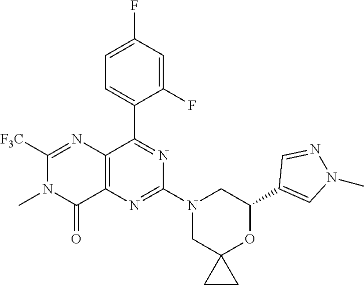 | methyl-6-(5-(1-methyl- 1H-pyrazol-4-yl)-4- oxa-7- azaspiro[2.5]octan- 7-yl)-2- (trifluoromethyl) pyrimido[5,4-d] pyrimidin- 4(3H)-one | | stereochemistry unknown |
| Ex. 166A (Peak 1) | (R)-6-(2,2-difluoro-6- | 6-(2,2-difluoro-6-(1- | Similar |
| | (1-methyl-1H-pyrazol- | methyl-1H-pyrazol-4- | specifications as |
| 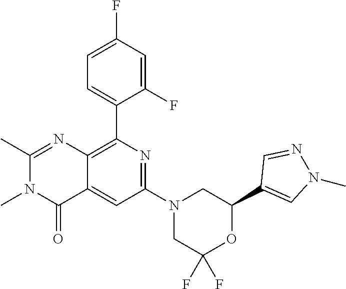 | 4-yl)morpholino)-8- (2,4-difluorophenyl)- 2,3-dimethylpyrido[3,4- d]pyrimidin-4(3H)-one | yl)morpholino)-8- (2,4-difluorophenyl)- 2,3- dimethylpyrido[3,4- d]pyrimidin-4(3H)- one (racemic compound prepared according to the | mentioned in step-6 of example 1 Peak 1 Absolute stereochemistry unknown |
| Ex. 166B (Peak 2) | (S)-6-(2,2-difluoro-6- | | Peak 2 |
| | (1-methyl-1H-pyrazol- | | Absolute |
| 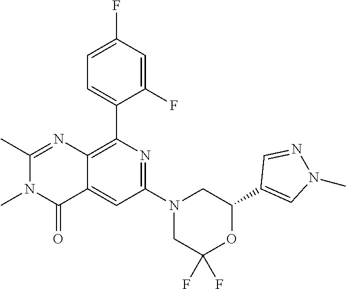 | 4-yl)morpholino)-8- (2,4-difluorophenyl)- 2,3-dimethylpyrido[3,4- d]pyrimidin-4(3H)-one | | stereochemistry unknown |

Following table describes analytical data analysis and yield information of examples 159A-1591B to examples 166A-166B:

| | | | Analytical | | Yield |
|---|---|---|---|---|---|
| Compound No. | HNMR | LCMS | chiral HPLC | Description | (%) |
| Ex. | ¹H NMR (400 MHZ, DMSO-d₆): | Condition | Chiralpak IG | Yellow solid | 38.5 |
| 159A | δ 8.42 (d, J = 6.0 Hz, 1 H), 7.60- | K: Rt | (4.6 × 250 mm), | | |
| (Peak 1) | 7.58 (m, 1 H), 7.36-7.33 (m, | 2.66 min. | 5 μ | | |
| | 3 H), 7.25 (d, J = 4.8 Hz, 1 H), | m/z 492.4 | ARD/K/ | | |
| | 7.19-7.18 (m, 1 H), 4.89 (d, J = | [M + H]⁺. | Mobile | | |
| | 8.8 Hz, 1 H), 4.44 (d, J = 12.0 Hz, | | Phase: | | |
| | 1 H), 4.20 (d, J = 12.8 Hz, 1 H), | | Hexane/DC | | |
| | 3.51 (s, 3 H), 2.81 (d, J = 12.8 | | M/EtOH/IPA | | |
| | Hz, 1 H), 2.66-2.61 (m, 1 H), | | mine: | | |
| | 2.47 (s, 3 H), 2.43 (s, 3 H), 1.32 | | 50/25/25/0.1 | | |
| | (s, 6 H). | | Flow Rate: | | |
| Ex. | ¹H NMR (400 MHZ, DMSO-d₆): | Condition | 1.0 ml/min | Yellow solid | 39.2 |
| 159B | δ 8.42 (d, J = 6.0 Hz, 1 H), 7.60- | K: Rt = | Solubility: | | |
| (Peak 2) | 7.58 (m, 1 H), 7.36-7.33 (m, | 2.66 min. | ACN, Rt = | | |
| | 3 H), 7.25 (d, J = 4.8 Hz, 1 H), | m/z 492.4 | 5.14 min for | | |
| | 7.19-7.18 (m, 1 H), 4.89 (d, J = | [M + H]⁺. | Peak 1 and | | |
| | 8.8 Hz, 1 H), 4.44 (d, J = 12.0 Hz, | | Rt = 5.96 min | | |
| | 1 H), 4.20 (d, J = 12.8 Hz, 1 H), | | for Peak 2. | | |
| | 3.51 (s, 3 H), 2.81 (d, J = 12.8 | | | | |
| | Hz, 1 H), 2.66-2.61 (m, 1 H), | | | | |
| | 2.47 (s, 3 H), 2.43 (s, 3 H), 1.32 | | | | |
| | (s, 6 H). | | | | |
| Ex. | ¹H NMR (400 MHZ, DMSO-d₆): | Condition | Chiralpak IG | Yellow solid | 43.1 |
| 160A | δ 8.42 (d, J = 4.8 Hz, 1 H), 7.67- | K: Rt = | (4.6 × 250 | | |
| (Peak 1) | 7.63 (m, 2 H), 7.39 (s, 1 H), 7.33 | 2.28 min. | mm), 5 μ | | |
| | (s, 1 H), 7.25 (d, J = 4.8 Hz, 1 H), | m/z 510.4 | ARD/K/ | | |
| | 4.88 (d, J = 10.4 Hz, 1 H), 4.47 | [M + H]⁺. | Mobile | | |
| | (d, J = 11.6 Hz, 1 H), 4.20 (d, | | Phase: | | |
| | J = 12.4 Hz, 1 H), 3.51 (s, 3 H), | | Hexane/DC | | |
| | 2.82 (d, J = 12.8 Hz, 1 H), 2.67- | | M/EtOH/IPAmine: | | |
| | 2.61 (m, 1 H), 2.47 (s, 3 H), 2.44 | | 50/25/25/0.1 | | |
| | (s, 3 H), 1.31 (s, 6 H). | | Flow Rate: | | |
| Ex. | ¹H NMR (400 MHZ, DMSO-d₆): | Condition | 1.0 ml/min | Yellow solid | 42.6 |
| 160B | δ 8.42 (d, J = 4.8 Hz, 1 H), 7.67- | K: Rt = | Solubility: | | |
| (Peak 2) | 7.63 (m, 2 H), 7.39 (s, 1 H), 7.33 | 2.28 min. | MeOH, Rt = | | |
| | (s, 1 H), 7.25 (d, J = 4.8 Hz, 1 H), | m/z 510.4 | 4.98 min for | | |
| | 4.88 (d, J = 10.4 Hz, 1 H), 4.47 | [M + H]⁺. | Peak 1 and | | |
| | (d, J = 11.6 Hz, 1 H), 4.20 (d, | | Rt = 5.75 min | | |
| | J = 12.4 Hz, 1 H), 3.51 (s, 3 H), | | for Peak 2. | | |
| | 2.82 (d, J = 12.8 Hz, 1 H), 2.67- | | | | |
| | 2.61 (m, 1 H), 2.47 (s, 3 H), 2.44 | | | | |
| | (s, 3 H), 1.31 (s, 6 H). | | | | |
| Ex. | ¹H NMR (400 MHZ, DMSO-d₆): | Condition | :CHIRALPAK IG | Yellow solid | 39.5 |
| 161A | δ 9.49 (s, 1 H), 8.79 (d, J = 8.0 | K: Rt = | (250 × 4.6) mm, | | |
| (Peak 1) | Hz, 1 H), 8.42 (d, J = 5.2 Hz, | 2.73 min. | 5 μ M.P.- | | |
| | 1 H), 8.03 (d, J = 8.4 Hz, 1 H), | m/z 525.4 | Hexane/DCM/ | | |
| | 7.44 (s, 1 H), 7.35 (s, 1 H), 7.29 | [M + H]⁺. | EtOH/IPAmine- | | |
| | (d, J = 5.2 Hz, 1 H), 4.91 (d, J = | | 50/25/25/0.1 | | |
| | 10.4 Hz, 1 H), 4.55 (d, J = 12.8 | | Flow Rate-1.0, | | |
| | Hz, 1 H), 4.30 (d, J = 12.0 Hz, | | solubility: | | |
| | 1 H), 3.54 (s, 3 H), 2.86 (d, | | MeOH, Rt= | | |
| | J = 12.8 Hz, 1 H), 2.72-2.67 (m, | | 4.51 min for | | |
| | 1 H), 2.54 (s, 3 H), 2.49 (s, 3 H), | | Peak 1 and | | |
| | 1.33 (s, 6 H). | | Rt = 4.92 min | | |
| Ex. | ¹H NMR (400 MHZ, DMSO-d₆): | Condition | for Peak 2. | Yellow solid | 38.6 |
| 161B | δ 9.49 (s, 1 H), 8.79 (d, J = 8.0 | K: Rt = | | | |
| (Peak 2) | Hz, 1 H), 8.42 (d, J = 5.2 Hz, | 2.73 min. | | | |
| | 1 H), 8.03 (d, J = 8.4 Hz, 1 H), | m/z 525.4 | | | |
| | 7.44 (s, 1 H), 7.35 (s, 1 H), 7.29 | [M + H]⁺. | | | |
| | (d, J = 5.2 Hz, 1 H), 4.91 (d, J = | | | | |
| | 10.4 Hz, 1 H), 4.55 (d, J = 12.8 | | | | |
| | Hz, 1 H), 4.30 (d, J = 12.0 Hz, | | | | |
| | 1 H), 3.54 (s, 3 H), 2.86 (d, | | | | |
| | J = 12.8 Hz, 1 H), 2.72-2.67 (m, | | | | |
| | 1 H), 2.54 (s, 3 H), 2.49 (s, 3 H), | | | | |
| | 1.33 (s, 6 H). | | | | |
| Ex. | ¹H NMR (400 MHZ, DMSO-d₆) | Condition K: | CHIRALPAK IG | Yellow solid | 38.2 |
| 162A | δ 7.84-7.76 (m, 3 H), 7.46 (s, | Rt = | (250 × 4.6) mm, | | |
| (Peak 1) | 1 H), 4.83 (bs, 1 H), 4.67-4.64 | 3.16 min. | 5 μ M.P.- | | |
| | (dd, J = 10.8 Hz, J = 2.4 Hz, 1 H), | m/z 552.3 | Hexane/DCM/ | | |
| | 4.17 (brs, 1 H), 3.81 (s, 3 H), | [M + H]⁺. | EtOH/IPAmine- | | |
| | 3.69 (d, J = 13.3 Hz, 1 H), 3.55 | | 50/25/25/0.1 | | |
| | (s, 3 H), 3.30 (brs, 1 H), 0.87 (bs, | | Flow Rate- | | |
| | 2 H), 0.71 (brs, 1 H), 0.58-0.53 | | 1.0 ml/min, | | |
| | (m, 1 H). | | solubility: | | |
| Ex. | ¹H NMR (400 MHZ, DMSO-d₆) | Condition | MeOH, Rt = | Yellow solid | 38.9 |
| 162B | δ 7.84-7.76 (m, 3 H), 7.46 (s, | K: Rt = | 4.46 min for | | |
| (Peak 2) | 1 H), 4.83 (bs, 1 H), 4.67-4.64 | 3.16 min. | Peak 1 and | | |
| | (dd, J = 10.8 Hz, J = 2.4 Hz, 1 H), | m/z 552.3 | Rt = 6.89 min | | |
| | 4.17 (brs, 1 H), 3.81 (s, 3 H), | [M + H]⁺. | for Peak 2. | | |
| | 3.69 (d, J = 13.3 Hz, 1 H), 3.55 | | | | |
| | (s, 3 H), 3.30 (brs, 1 H), 0.87 | | | | |
| | (brs, 2 H), 0.71 (brs, 1 H), 0.58- | | | | |
| | 0.53 (m, 1 H). | | | | |
| Ex. | ¹H NMR (400 MHZ, DMSO-d₆): | Condition | CHIRALPAK IG | Yellow solid | 41.5 |
| 163A | δ 8.42 (d, J = 5.2 Hz, 1 H), 7.80- | K: Rt = | (250 × 4.6) mm, | | |
| (Peak 1) | 7.76 (m, 2 H), 7.69 (d, J = 8.0 | 2.86 min. | 5 μ M.P.- | | |
| | Hz, 1 H), 7.42 (s, 1 H), 7.35 (s, | m/z 542.4 | Hexane/DCM/ | | |
| | 1 H), 7.27 (d, J = 5.2 Hz, 1 H), | [M + H]⁺. | EtOH/IPAmine- | | |
| | 4.90 (d, J = 10.4 Hz, 1 H), 4.45 | | 60/20/20/0.1 | | |
| | (d, J = 12.0 Hz, 1 H), 4.22 (d, | | Flow Rate- | | |
| | J = 12.4 Hz, 1 H), 3.52 (s, 3 H), | | 1.0 ml/min | | |
| | 2.82 (d, J = 12.8 Hz, 1 H), 2.68- | | Solubility : | | |
| | 2.62 (m, 1 H), 2.48 (s, 3 H), 2.43 | | MeOH, Rt = | | |
| | (s, 3 H), 1.32 (s, 6 H). | | 4.82 min for | | |
| Ex. | ¹H NMR (400 MHZ, DMSO-d₆): | Condition | Peak 1 and | Yellow solid | 40.2 |
| 163B | δ 8.42 (d, J = 5.2 Hz, 1 H), 7.80- | K: Rt = | Rt = 5.09 min | | |
| (Peak 2) | 7.76 (m, 2 H), 7.69 (d, J = 8.0 | 2.86 min. | for Peak 2. | | |
| | Hz, 1 H), 7.42 (s, 1 H), 7.35 (s, | m/z 542.4 | | | |
| | 1 H), 7.27 (d, J = 5.2 Hz, 1 H), | [M + H]⁺. | | | |
| | 4.90 (d, J = 10.4 Hz, 1 H), 4.45 | | | | |
| | (d, J = 12.0 Hz, 1 H), 4.22 (d, | | | | |
| | J = 12.4 Hz, 1 H), 3.52 (s, 3 H), | | | | |
| | 2.82 (d, J = 12.8 Hz, 1 H), 2.68- | | | | |
| | 2.62 (m, 1 H), 2.48 (s, 3 H), 2.43 | | | | |
| | (s, 3 H), 1.32 (s, 6 H). | | | | |
| Ex. | ¹H NMR (400 MHZ, DMSO-d₆): | Condition | CHIRALPAK IG | Yellow solid | 39.1 |
| 164A | δ 9.59 (s, 1 H), 8.92 (d, J = 8.0 | K: Rt = | (250 × 4.6) mm, | | |
| (Peak 1) | Hz, 1 H), 8.45 (d, J = 5.2 Hz, | 2.87 min. | 5 μ M.P.- | | |
| | 1 H), 8.09 (d, J = 8.4 Hz, 1 H), | m/z 526.3 | Hexane/DCM/ | | |
| | 7.34 (s, 1 H), 7.27 (d, J = 4.8 Hz, | [M + H]⁺. | EtOH/IPAmine- | | |
| | 1 H), 4.91-4.86 (m, 2 H), 4.65 | | 70/15/15/0.1 | | |
| | (d, J = 13.2 Hz, 1 H), 3.54 (s, | | Flow Rate- | | |
| | 3 H), 3.00 (d, J = 13.2 Hz, 1 H), | | 1.0 ml/min: | | |
| | 2.89-2.83 (m, 1 H), 2.55 (s, 3 H), | | Solubility: | | |
| | 2.51 (s, 3 H), 1.37 (s, 3 H), 1.29 | | MeOH, Rt = | | |
| | (s, 3 H). | | 10.92 min for | | |
| Ex. | ¹H NMR (400 MHZ, DMSO-d₆): | Condition | Peak 1 and | Yellow solid | 38.6 |
| 164B | δ 9.59 (s, 1 H), 8.92 (d, J = 8.0 | K: Rt = | Rt = 11.95 | | |
| (Peak 2) | Hz, 1 H), 8.45 (d, J = 5.2 Hz, | 2.87 min. | min for Peak 2. | | |
| | 1 H), 8.09 (d, J = 8.4 Hz, 1 H), | m/z 526.3 | | | |
| | 7.34 (s, 1 H), 7.27 (d, J = 4.8 Hz, | [M + H]⁺. | | | |
| | 1 H), 4.91-4.86 (m, 2 H), 4.65 | | | | |
| | (d, J = 13.2 Hz, 1 H), 3.54 (s, | | | | |
| | 3 H), 3.00 (d, J = 13.2 Hz, 1 H), | | | | |
| | 2.89-2.83 (m, 1 H), 2.55 (s, 3 H), | | | | |
| | 2.51 (s, 3 H), 1.37 (s, 3 H), 1.29 | | | | |
| | (s, 3 H). | | | | |
| Ex. | 1H NMR (400 MHZ, DMSO-d₆) | Condition | CHIRALPAK IG | Yellow solid | 39.5 |
| 165A | δ 7.75 (brs, 2 H), 7.47-7.43 (m, | K: Rt = | (250 × 4.6) mm, | | |
| (Peak 1) | 2 H), 7.30-7.26 (m, 1 H), 4.84- | 3.11 min. | 5 μ M.P.- | | |
| | 4.79 (m, 1 H), 4.64 (d, J = 10.8 | m/z 534.3 | Hexane/DCM/ | | |
| | Hz, 1 H), 4.16 (brs, 1 H), 3.81 (s, | [M + H]⁺. | EtOH/IPAmine- | | |
| | 3 H), 3.69 (d, J = 13.6 Hz, 1 H), | | 50/25/25/0.1 | | |
| | 3.55 (s, 3 H), 0.87 (bs, 2 H), 0.72 | | Flow Rate | | |
| | (brs, 1 H), 0.55 (brs, 1 H). | | 1.0 ml/min, | | |
| Ex. | 1H NMR (400 MHZ, DMSO-d₆) | Condition | solubility: | Yellow solid | 38.6 |
| 165B | δ 7.75 (brs, 2 H), 7.47-7.43 (m, | K: Rt = | MeOH, Rt = | | |
| (Peak 2) | 2 H), 7.30-7.26 (m, 1 H), 4.84- | 3.11 min. | 4.53 min for | | |
| | 4.79 (m, 1 H), 4.64 (d, J = 10.8 | m/z 534.3 | Peak 1 and | | |
| | Hz, 1 H), 4.16 (brs, 1 H), 3.81 (s, | [M + H]⁺. | Rt = 7.65 min | | |
| | 3 H), 3.69 (d, J = 13.6 Hz, 1 H), | | for Peak 2. | | |
| | 3.55 (s, 3 H), 0.87 (bs, 2 H), 0.72 | | | | |
| | (brs, 1 H), 0.55 (brs, 1 H). | | | | |
| Ex. | ¹H-NMR (400 MHZ, DMSO-d6): | Condition | CHIRALPAK IG | Yellow solid | 40.2 |
| 166A | δ 7.90 (s, 1 H), 7.63-7.61 (m, | K: Rt = | (250 × 4.6) mm, | | |
| (Peak 1) | 1 H), 7.59 (s, 1 H), 7.46 (s, 1 H), | 3.01 min. | 5 μ M.P.- | | |
| | 7.37-7.32 (m, 1 H), 7.22-7.18 | m/z 489.3 | Hexane/DCM/ | | |
| | (m, 1 H), 5.31-5.27 (dd, J = 11.2 | [M + H]⁺. | EtOH/IPAmine- | | |
| | Hz, J = 2.4 Hz, 1 H), 4.70 (d, J = | | 60/20/20/0.1 | | |
| | 13.2 Hz, 1 H), 4.42 (d, J = 13.2 | | Flow Rate- | | |
| | Hz, 1 H), 3.84 (s, 3 H), 3.62-3.56 | | 1.0 ml/min, | | |
| | (m, 1 H), 3.52 (s, 3 H), 3.38-3.32 | | solubility: | | |
| | (m, 1 H), 2.45 (s, 3 H). | | MeOH, Rt= | | |
| Ex. | ¹H-NMR (400 MHZ, DMSO-d6): | Condition | 11.71 min for | Yellow solid | 39.1 |
| 166B | δ 7.90 (s, 1 H), 7.63-7.61 (m, | K: Rt = | Peak 1 and | | |
| (Peak 2) | 1 H), 7.59 (s, 1 H), 7.46 (s, 1 H), | 3.01 min. | Rt = 14.92 | | |
| | 7.37-7.32 (m, 1 H), 7.22-7.18 | m/z 489.3 | min for Peak 2. | | |
| | (m, 1 H), 5.31-5.27 (dd, J = 11.2 | [M + H]⁺. | | | |
| | Hz, J = 2.4 Hz, 1 H), 4.70 (d, J = | | | | |
| | 13.2 Hz, 1 H), 4.42 (d, J = 13.2 | | | | |
| | Hz, 1 H), 3.84 (s, 3 H), 3.62-3.56 | | | | |
| | (m, 1 H), 3.52 (s, 3 H), 3.38-3.32 | | | | |
| | (m, 1 H), 2.45 (s, 3 H). | | | | |

### Example 167

### 6-(2-(1-cyclopropyl-1H-pyrazol-4-yl)tetrahydro-2H-pyran-4-yl)-8-(2,4-difluorophenyl)-5-fluoro-3-methyl-2-(trifluoromethyl) pyrido[3,4-d]pyrimidin-4 (3H)-one

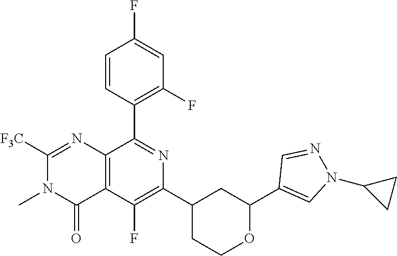 [SMILES: Cn1c(C(F)(F)F)nc2c(-c3ccc(F)cc3F)nc(C3CCOC(c4cnn(C5CC5)c4)C3)c(F)c2c1=O]

Synthesized using similar protocol as described in example 157 and followed by prep HPLC purification to afford 6-(2-(1-cyclopropyl-1H-pyrazol-4-yl)tetrahydro-2H-pyran-4-yl)-8-(2,4-difluorophenyl)-5-fluoro-3-methyl-2-(trifluoromethyl)pyrido[3,4-d]pyrimidin-4 (3H)-one (17 mg, 15% yield) as white solid.

Prep-HPLC: Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 40% A and 60% B, then 10% A and 90% B in 22 min, then to 5% A and 95% B in 23 min., held this composition up to 25 min. for column washing, then returned to initial composition in 26 min. and held till 28 min.

1H NMR (400 MHz, DMSO-d6): δ 7.69 (s, 1H), 7.67-7.61 (m, 1H), 7.43-7.37 (m, 1H), 7.35 (s, 1H), 7.28-7.23 (m, 1H), 4.51 (d, J=10.8 Hz, 1H), 4.08-4.05 (m, 1H), 3.75-3.62 (m, 3H), 3.54 (s, 3H), 2.02-1.86 (m, 3H), 1.73 (d, J=12.4 Hz, 1H), 0.99-0.95 (m, 2H), 0.93-0.88 (m, 2H).

LCMS Condition K: Rt=3.20 min. m/z 550.3 [M+H]⁺.

### Example 168

### Example 168A: Enantiomer Peak 1

### 8-(2-fluoro-4-(trifluoromethyl)phenyl)-2,3-dimethyl-6-((2S,4R)-2-(2-methylpyridin-4-yl)tetrahydro-2H-pyran-4-yl)pyrido[3,4-d]pyrimidin-4 (3H)-one

### Example 168B: Enantiomer Peak 2

### 8-(2-fluoro-4-(trifluoromethyl)phenyl)-2,3-dimethyl-6-((2R,4S)-2-(2-methylpyridin-4-yl)tetrahydro-2H-pyran-4-yl)pyrido[3,4-d]pyrimidin-4 (3H)-one

### Step-1—Preparation of 4-(4-bromotetrahydro-2H-pyran-2-yl)-2-methylpyridine

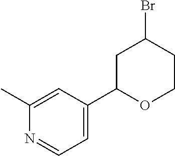

4-(4-bromotetrahydro-2H-pyran-2-yl)-2-methylpyridine was synthesized following the procedure as described in Step-1, Example 94.

### Step-2 Preparation of 8-(2-fluoro-4-(trifluoromethyl)phenyl)-2,3-dimethyl-6-(2-(2-methylpyridin-4-yl)tetrahydro-2H-pyran-4-yl)pyrido[3,4-d]pyrimidin-4 (3H)-one

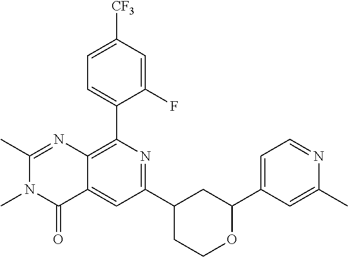

To a stirred solution of 4-(4-bromotetrahydro-2H-pyran-2-yl)-2-methylpyridine (130 mg, 0.51 mmol) in DMA (5 mL) under argon atmosphere were added NaI (20 mg, 0.12 mmol), 4,4,4″-tri-tert-butyl-2,2,6,2-terpyridine (14 mg, 0.04 mmol), NiCl₂(DME) (8 mg, 0.04 mmol), Zinc (66 mg, 1.01 mmol) and 6-chloro-8-(2-fluoro-4-(trifluoromethyl)phenyl)-2,3-dimethylpyrido[3,4-d]pyrimidin-4 (3H)-one (190 mg, 0.51 mmol) successively. Finally, TFA (0.004 mL, 0.51 mmol) was added to the reaction mixture and the resulting mixture was heated at 60° C. for 16 h. After completion, reaction mixture was filtered through a pad of celite, washed with dichloromethane and concentrated under reduced pressure. Crude product was purified by reverse phase preparative HPLC to 8-(2-fluoro-4-(trifluoromethyl)phenyl)-2,3-dimethyl-6-(2-(2-methylpyridin-4-yl)tetrahydro-2H-pyran-4-yl)pyrido[3,4-d]pyrimidin-4 (3H)-one (50 mg, 19% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 45% A and 55% B, then 45% A and 55% B in 3 min, then to 25% A and 75% B in 20 min., then to 5% A and 95% B in 21 min., held this composition up to 22 min. for column washing, then returned to initial composition in 23 min. and held till 25 min.

1H NMR (400 MHz, DMSO-d6) δ 8.34 (d, J=5.2 Hz, 1H), 7.95 (s, 1H), 7.81-7.75 (m, 2H), 7.70 (d, J=8.0 Hz, 1H), 7.25 (s, 1H), 7.16 (d, J=4.8 Hz, 1H), 4.53 (d, J=11.2 Hz, 1H), 4.22-4.19 (m, 1H), 3.76-3.71 (m, 1H), 3.54 (s, 3H), 3.31-3.29 (m, 1H), 2.49 (s, 3H), 2.44 (s, 3H), 2.24-2.21 (m, 1H), 1.97-1.86 (m, 2H), 1.69-1.63 (m, 1H).

### Condition K: Rt=2.62 min. m/z 513.4 [M+H]⁺

### Step-3—Preparation of 8-(2-fluoro-4-(trifluoromethyl)phenyl)-2,3-dimethyl-6-((2S,4R)-2-(2-methylpyridin-4-yl)tetrahydro-2H-pyran-4-yl)pyrido[3,4-d]pyrimidin-4 (3H)-one [Example 168A] and 8-(2-fluoro-4-(trifluoromethyl)phenyl)-2,3-dimethyl-6-((2R,4S)-2-(2-methylpyridin-4-yl)tetrahydro-2H-pyran-4-yl)pyrido[3,4-d]pyrimidin-4 (3H)-one [Example 168B]

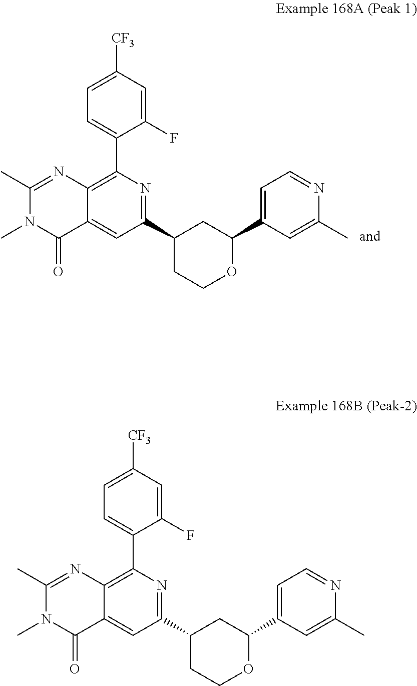

Chiral separation of 8-(2-fluoro-4-(trifluoromethyl)phenyl)-2,3-dimethyl-6-(2-(2-methylpyridin-4-yl)tetrahydro-2H-pyran-4-yl)pyrido[3,4-d]pyrimidin-4 (3H)-one (50.0 mg by normal phase chiral prep HPLC afforded Example 168B, 8-(2-fluoro-4-(trifluoromethyl)phenyl)-2,3-dimethyl-6-((2S,4R)-2-(2-methylpyridin-4-yl)tetrahydro-2H-pyran-4-yl)pyrido[3,4-d]pyrimidin-4 (3H)-one (11 mg, 11% yield; eluted earlier, assigned as Peak 1 with arbitrary assignment of stereochemistry) as yellow solid and Example 168B, 8-(2-fluoro-4-(trifluoromethyl)phenyl)-2,3-dimethyl-6-((2R,4S)-2-(2-methylpyridin-4-yl)tetrahydro-2H-pyran-4-yl)pyrido[3,4-d]pyrimidin-4 (3H)-one (10 mg, 10% yield; eluted later, assigned as Peak 2 with arbitrary assignment of stereochemistry) as yellow solid.

Example 168A (Peak 1): 1H NMR (400 MHz, DMSO-d6) δ 8.34 (d, J=5.2 Hz, 1H), 7.95 (s, 1H), 7.81-7.75 (m, 2H), 7.70 (d, J=8.0 Hz, 1H), 7.25 (s, 1H), 7.16 (d, J=4.8 Hz, 1H), 4.53 (d, J=11.2 Hz, 1H), 4.22-4.19 (m, 1H), 3.76-3.71 (m, 1H), 3.54 (s, 3H), 3.31-3.29 (m, 1H), 2.49 (s, 3H), 2.44 (s, 3H), 2.24-2.21 (m, 1H), 1.97-1.86 (m, 2H), 1.69-1.63 (m, 1H).

Condition K: Rt=2.62 min. m/z 513.4 [M+H]⁺

Example 168B (Peak 2): 1H NMR (400 MHz, DMSO-d6) δ 8.34 (d, J=5.2 Hz, 1H), 7.95 (s, 1H), 7.81-7.75 (m, 2H), 7.70 (d, J=8.0 Hz, 1H), 7.25 (s, 1H), 7.16 (d, J=4.8 Hz, 1H), 4.53 (d, J=11.2 Hz, 1H), 4.22-4.19 (m, 1H), 3.76-3.71 (m, 1H), 3.54 (s, 3H), 3.31-3.29 (m, 1H), 2.49 (s, 3H), 2.44 (s, 3H), 2.24-2.21 (m, 1H), 1.97-1.86 (m, 2H), 1.69-1.63 (m, 1H).

### Condition K: Rt=2.62 min. m/z 513.4 [M+H]⁺

Analytical chiral HPLC: CHIRALPAK IG (250×4.6) mm, 5μ M.P.—Hexane/DCM/EtOH/IPAmine: 50/25/25/0.1, Flow Rate—1.0 ml/min. Solubility: MeOH Rt=4.64 min for Peak 1 and Rt=5.05 min for Peak 2.

### Example 169

### Example 169A: Enantiomer Peak 1

### (R)-6-(2,2-difluoro-6-(2-methylpyridin-4-yl)morpholino)-8-(2,4-difluorophenyl)-2,3-dimethylpyrimido[5,4-d]pyrimidin-4 (3H)-one

### Example 169B: Enantiomer Peak 2

### (S)-6-(2,2-difluoro-6-(2-methylpyridin-4-yl)morpholino)-8-(2,4-difluorophenyl)-2,3-dimethylpyrimido[5,4-d]pyrimidin-4 (3H)-one

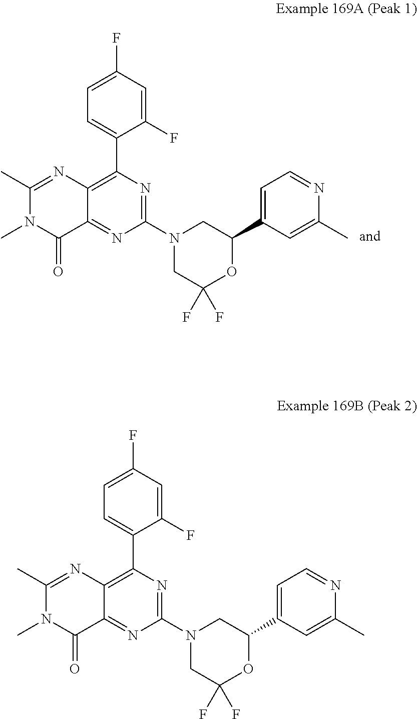

6-(2,2-difluoro-6-(2-methylpyridin-4-yl)morpholino)-8-(2,4-difluorophenyl)-2,3-dimethylpyrimido[5,4-d]pyrimidin-4 (3H)-one (70 mg, 30% yield, white solid) was synthesized according to similar procedure depicted in Example 115 and where corresponding 2,2-difluoro-6-(2-methylpyridin-4-yl)morpholine was synthesized using lithium aluminium hydride (1.5 eq.) instead of boron dimethyl sulphide complex. Chiral separation of 6-(2,2-difluoro-6-(2-methylpyridin-4-yl)morpholino)-8-(2,4-difluorophenyl)-2,3-dimethylpyrimido[5,4-d]pyrimidin-4 (3H)-one (70 mg) was done by normal phase chiral prep HPLC to afford Example 169A, (R)-6-(2,2-difluoro-6-(2-methylpyridin-4-yl)morpholino)-8-(2,4-difluorophenyl)-2,3-dimethylpyrimido[5,4-d]pyrimidin-4 (3H)-one (28 mg, 39%; eluted first, assigned as Peak 1 with arbitrary assignment of stereochemistry) as white solid and Example 169B, (S)-6-(2,2-difluoro-6-(2-methylpyridin-4-yl)morpholino)-8-(2,4-difluorophenyl)-2,3-dimethylpyrimido[5,4-d]pyrimidin-4 (3H)-one (25.0 mg, 35% yield, eluted later, assigned as Peak 2 with arbitrary assignment of stereochemistry) as white solid.

Example 169A (Peak 1): 1H NMR (400 MHz, DMSO-d6) δ 8.51 (d, J=5.2 Hz, 1H), 7.76-7.75 (m, 1H), 7.44-7.43 (m, 1H), 7.39 (s, 1H), 7.32-7.27 (m, 2H), 5.40-5.3d (dd, J=11.2 Hz, J=2.8 Hz, 1H), 4.99 (d, J=13.2 Hz, 1H), 4.77 (d, J=13.6 Hz, 1H), 3.86-3.76 (m, 1H), 3.53 (s, 3H), 3.38-3.35 (m, 1H), 2.49 (s, 3H), 2.46 (s, 3H).

LCMS Condition K: Rt=2.80 min. m/z 501.2 [M+H]⁺.

Example 169B (Peak 2): 1H NMR (400 MHz, DMSO-d6) δ 8.51 (d, J=5.2 Hz, 1H), 7.76-7.75 (m, 1H), 7.44-7.43 (m, 1H), 7.39 (s, 1H), 7.32-7.27 (m, 2H), 5.40-5.33 (dd, J=11.2 Hz, J=2.8 Hz, 1H), 4.99 (d, J=13.2 Hz, 1H), 4.77 (d, J=13.6 Hz, 1H), 3.86-3.76 (m, 1H), 3.53 (s, 3H), 3.38-3.35 (m, 1H), 2.49 (s, 3H), 2.46 (s, 3H).

LCMS Condition K: Rt=2.80 min. m/z 501.2 [M+H]⁺.

Analytical chiral HPLC: CHIRALPAK IG (250×4.6) mm, 5μ M.P.—Hexane/DCM/EtOH/IPAmine: 50/25/25/0.1 Flow Rate—1.0 ml/min, solubility: MeOH, Rt=5.46 min for Peak 1 and Rt=6.93 min for Peak 2.

### Example 170 to Example 173

Example 170 [A-B] to Example 173[A-B]were synthesized as similar procedure as Example 112. Corresponding chloro intermediates were synthesized following same procedure as Example 112, step-1.

Chiral separation details of Example 170A-170B to 173A-173B are captured in following

| | | | | |
|---|---|---|---|---|
| | | | | Chiral |
| Comp- | | | | separation |
| ound No. | Structure | Name | Starting material | specification |

| | | | |
|---|---|---|---|
| Ex. 170A (Peak 1) | (R)-8-(2,4- | 8-(2,4- | Peak 1 |
| | difluorophenyl)-6- | difluorophenyl)-6- | Absolute |
| 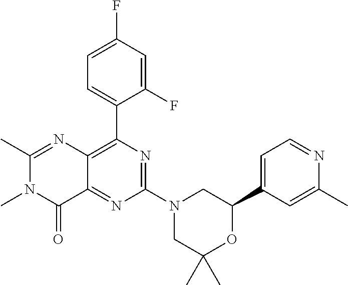 | (2,2-dimethyl-6-(2- methylpyridin-4- yl)morpholino)-2,3- dimethylpyrimido [5,4- d]pyrimidin-4(3H)- one | (2,2-dimethyl-6-(2- methylpyridin-4- | stereochemistry unknown |
| Ex. 170B (Peak 2) | (S)-8-(2,4- | yl)morpholino)-2,3- | Peak 2 |
| | difluorophenyl)-6- | dimethylpyrimido[5, | Absolute |
| 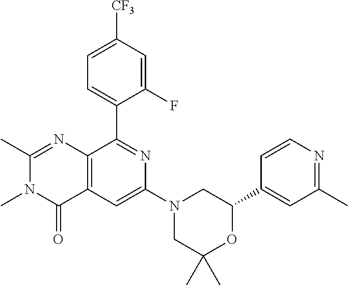 | (2,2-dimethyl-6-(2- methylpyridin-4- yl)morpholino)-2,3- dimethylpyrimido [5,4- d]pyrimidin-4(3H)- one | 4-d]pyrimidin- 4(3H)-one | stereochemistry unknown. |
| Ex. 171A (Peak 1) | (R)-6-(2,2-difluoro-6- | 6-(2,2-difluoro-6-(2- | Peak 1 |
| | (2-methylpyridin-4- | methylpyridin-4- | Absolute |
| 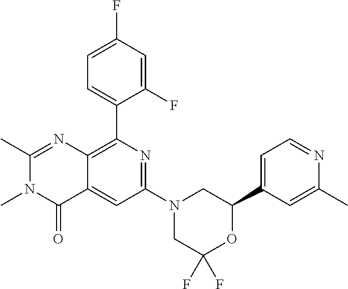 | yl)morpholino)-8- (2,4-difluorophenyl)- 2,3- dimethylpyrido[3,4- d]pyrimidin-4(3H)- one | yl)morpholino)-8- (2,4- difluorophenyl)-2,3- dimethylpyrido[3,4- d]pyrimidin-4(3H)- one | stereochemistry unknown |
| Ex. 171B (Peak 2) | (S)-6-(2,2-difluoro-6- | | Peak 2 |
| | (2-methylpyridin-4- | | Absolute |
| 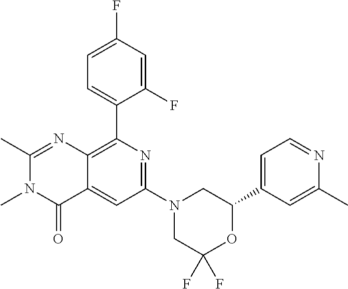 | yl)morpholino)-8- (2,4-difluorophenyl)- 2,3- dimethylpyrido[3,4- d]pyrimidin-4(3H)- one | | stereochemistry unknown |
| Ex. 172A (Peak 1) | (R)-8-(4-chloro-2- | 8-(4-chloro-2- | Peak 1 |
| | fluorophenyl)-6-(2,2- | fluorophenyl)-6- | Absolute |
| 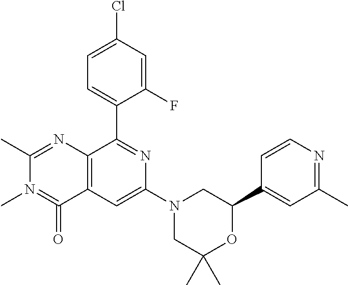 | dimethyl-6-(2- methylpyridin-4- yl)morpholino)-2,3- dimethylpyrido[3,4- d]pyrimidin-4(3H)- one | (2,2-dimethyl-6-(2- methylpyridin-4- yl)morpholino)-2,3- dimethylpyrido[3,4- d]pyrimidin-4(3H)- one | stereochemistry unknown |
| Ex. 172B (Peak 2) | (S)-8-(4-chloro-2- | | Peak 2 |
| | fluorophenyl)-6-(2,2- | | Absolute |
| 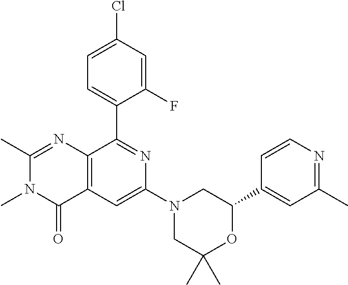 | dimethyl-6-(2- methylpyridin-4- yl)morpholino)-2,3- dimethylpyrido[3,4- d]pyrimidin-4(3H)- one | | stereochemistry unknown |
| Ex. 173A (Peak 1) | (R)-8-(4-chloro-2- | 8-(4-chloro-2- | Peak 1 |
| | fluorophenyl)-5- | fluorophenyl)-5- | Absolute |
| 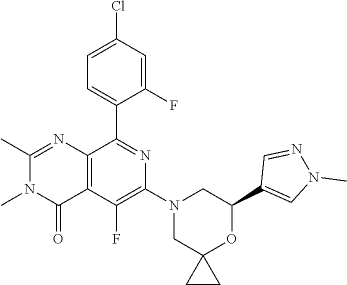 | fluoro-2,3-dimethyl-6- (5-(1-methyl-1H- pyrazol-4-yl)-4-oxa- 7-azaspiro[2.5]octan- 7-yl)pyrido[3,4- d]pyrimidin-4(3H)- one | fluoro-2,3-dimethyl- 6-(5-(1-methyl-1H- pyrazol-4-yl)- 4-oxa-7- azaspiro[2.5]octan- 7-yl)pyrido[3,4- d]pyrimidin-4(3H)- one [racemate was synthesized in Ex- 157] | stereochemistry unknown |
| Ex. 173B (Peak 2) | (S)-8-(4-chloro-2- | | Peak 2 |
| | fluorophenyl)-5- | | Absolute |
| 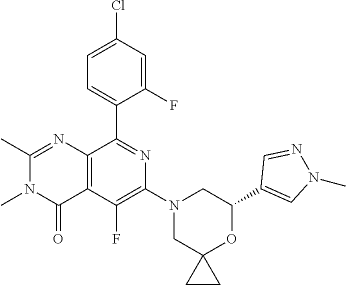 | fluoro-2,3-dimethyl-6- (5-(1-methyl-1H- pyrazol-4-yl)-4-oxa- 7-azaspiro[2.5]octan- 7-yl)pyrido[3,4- d]pyrimidin-4(3H)- one | | stereochemistry unknown |

Following table describes analytical data analysis and yield information of examples 170A-170B to examples 173A-173B:

| | | | Analytical | | Yield |
|---|---|---|---|---|---|
| Compound No. | HNMR | LCMS | chiral HPLC | Description | (%) |
| Ex. 170A | ¹H NMR (400 MHZ, DMSO-d₆): | Condition K: | I-CELLULOSE | yellow | 41.5 |
| (Peak 1) | δ 8.45 (d, J = 4.8 Hz, | Rt = 2.50 | Z(150 × 4.6) mm, | solid | |
| | 1 H), 7.74-7.68 (m, 1 H), | min. m/z | 3 μ M.P.-0.3% | | |
| | 7.44-7.38 (m, 1 H), 7.31 (s, | 493.5 [M + H]⁺. | IPamine | | |
| | 1 H), 7.28-7.24 (m, 2 H), | | in MeOH: | | |
| | 4.90-4.86 (dd, J = 10.8 Hz, | | Flow Rate-4.0 | | |
| | J = 2.4 Hz, 1 H), 4.77 (d, | | ml/min Solubility: | | |
| | J = 12.0 Hz, 1 H), 4.55 (d, | | MeOH, Rt = | | |
| | J = 12.8 Hz, 1 H), 3.51 (s, | | 1.33 min for | | |
| | 3 H), 2.97 (d, J = 13.2 Hz, | | Peak 1 and Rt = | | |
| | 1 H), 2.82 (t, J = 12.0 Hz, 1 H), | | 1.48 min for | | |
| | 2.49 (s, 3 H), 2.44 (s, | | Peak 2. | | |
| | 3 H), 1.34 (s, 3 H), 1.27 (s, | | | | |
| | 3 H). | | | | |
| Ex. 170B | ¹H NMR (400 MHZ, DMSO-d₆): | Condition K: | | yellow | 40.2 |
| (Peak 2) | δ 8.45 (d, J = 4.8 Hz, | Rt = 2.50 | | solid | |
| | 1 H), 7.74-7.68 (m, 1 H), | min. m/z | | | |
| | 7.44-7.38 (m, 1 H), 7.31 (s, | 493.5 [M + H]⁺. | | | |
| | 1 H), 7.28-7.24 (m, 2 H), | | | | |
| | 4.90-4.86 (dd, J = 10.8 Hz, | | | | |
| | J = 2.4 Hz, 1 H), 4.77 (d, | | | | |
| | J = 12.0 Hz, 1 H), 4.55 (d, | | | | |
| | J = 12.8 Hz, 1 H), 3.51 (s, | | | | |
| | 3 H), 2.97 (d, J = 13.2 Hz, | | | | |
| | 1 H), 2.82 (t, J = 12.0 Hz, 1 H), | | | | |
| | 2.49 (s, 3 H), 2.44 (s, | | | | |
| | 3 H), 1.34 (s, 3 H), 1.27 (s, | | | | |
| | 3 H). | | | | |
| Ex. 171A | ¹H NMR (400 MHZ, DMSO-d₆) | Condition K: | CHIRALPAK IG | white solid | 39.1 |
| (Peak 1) | δ 8.50 (d, J = 4.8 Hz, | Rt = 2.75 | (250 × 4.6) mm, | | |
| | 1 H), 7.67-7.61 (m, 1 H), 7.53 | min. m/z | 5 μ M.P.- | | |
| | (s, 1 H), 7.41 (s, 1 H), 7.38- | 500.4 [M + H]⁺. | Hexane/DCM/ | | |
| | 7.34 (m, 2 H), 7.23-7.19 (m, | | EtOH/IPAmine- | | |
| | 1 H), 5.38 (d, J = 8.8 Hz, 1 H), | | 50/25/25/0.1 | | |
| | 4.80 (d, J = 12.8 Hz, 1 H), | | Flow Rate-1.0 | | |
| | 4.53 (d, J = 12.8 Hz, 1 H), | | ml/min: | | |
| | 3.65-3.58 (m, 1 H), 3.53 (s, | | Solubility: | | |
| | 3 H), 3.22-3.16 (m, 1 H), | | MeOH, Rt = | | |
| | 2.50 (s, 3 H), 2.45 (s, 3 H). | | 5.89 min for | | |
| | | | Peak 1 and Rt = | | |
| Ex. 171B | ¹H NMR (400 MHZ, DMSO-d₆): | Condition K: | 7.73 min for | white solid | 38.6 |
| (Peak 2) | δ 8.50 (d, J = 4.8 Hz, | Rt = 2.75 | Peak 2. | | |
| | 1 H), 7.67-7.61 (m, 1 H), 7.53 | min. m/z | | | |
| | (s, 1 H), 7.41 (s, 1 H), 7.38- | 500.4 [M + H]⁺. | | | |
| | 7.34 (m, 2 H), 7.23-7.19 (m, | | | | |
| | 1 H), 5.38 (d, J = 8.8 Hz, 1 H), | | | | |
| | 4.80 (d, J = 12.8 Hz, 1 H), | | | | |
| | 4.53 (d, J = 12.8 Hz, 1 H), | | | | |
| | 3.65-3.58 (m, 1 H), 3.53 (s, | | | | |
| | 3 H), 3.22-3.16 (m, 1 H), | | | | |
| | 2.50 (s, 3 H), 2.45 (s, 3 H). | | | | |
| Ex. 172A | 1H NMR (400 MHZ, DMSO-d₆) | Condition K: | I-CELLULOSE | Yellow | 39.5 |
| (Peak 1) | δ 8.42 (d, J = 4.8 Hz, 1 H), | Rt = 2.69 | Z(150 × 4.6) mm, | solid | |
| | 7.59-7.7.52 (m, 2 H), 7.41- | min. m/z | 3 μ M.P.-0.3% | | |
| | 7.39 (m, 1 H), 7.38 (s, 1 H), | 508.4 [M + H]⁺. | IPamine | | |
| | 7.32 (s, 1 H), 7.25 (d, J = 5.2 | | in MeOH: | | |
| | Hz, 1 H), 4.91-4.88 (dd, J = | | Flow Rate-4.0 | | |
| | 10.8 Hz, J = 2.4 Hz, 1 H), | | ml/min Solubility: | | |
| | 4.44 (d, J = 11.6 Hz, 1 H), | | MeOH, Rt = | | |
| | 4.20 (d, J = 12.8 Hz, 1 H), | | 1.39 min for | | |
| | 3.51 (s, 3 H), 2.81 (d, J = 12.8 | | Peak 1 and Rt = | | |
| | Hz, 1 H), 2.67-2.61 (m, 1 H), | | 1.56 min for | | |
| | 2.47 (s, 3 H), 2.43 (s, 3 H), | | Peak 2. | | |
| | 1.32 (s, 6 H). | | | | |
| Ex. 172B | 1H NMR (400 MHZ, DMSO-d⁶) | Condition K: | | Yellow | 38.6 |
| (Peak 2) | δ 8.42 (d, J = 4.8 Hz, 1 H), | Rt = 2.69 | | solid | |
| | 7.59-7.7.52 (m, 2 H), 7.41- | min. m/z | | | |
| | 7.39 (m, 1 H), 7.38 (s, 1 H), | 508.4 [M + H]⁺. | | | |
| | 7.32 (s, 1 H), 7.25 (d, J = 5.2 | | | | |
| | Hz, 1 H), 4.91-4.88 (dd, J = | | | | |
| | 10.8 Hz, J = 2.4 Hz, 1 H), | | | | |
| | 4.44 (d, J = 11.6 Hz, 1 H), | | | | |
| | 4.20 (d, J = 12.8 Hz, 1 H), | | | | |
| | 3.51 (s, 3 H), 2.81 (d, J = 12.8 | | | | |
| | Hz, 1 H), 2.67-2.61 (m, 1 H), | | | | |
| | 2.47 (s, 3 H), 2.43 (s, 3 H), | | | | |
| | 1.32 (s, 6 H). | | | | |
| Ex. 173A | ¹H-NMR (400 MHZ, DMSO-d6): | Condition K: | CHIRALPAK IG | Yellow | 40.2 |
| (Peak 1) | δ 7.69 (s, 1 H), 7.57- | Rt = 3.31 | (250 × 4.6) mm, | solid | |
| | 7.52 (m, 2 H), 7.41-7.39 (m, | min. m/z | 5 μ M.P.- | | |
| | 2 H), 4.79 (d, J = 10.8 Hz, | 513.2 [M + H]⁺. | Hexane/DCM/ | | |
| | 1 H), 3.99 (d, J = 13.6 Hz, | | EtOH/IPAmine: | | |
| | 1 H), 3.78 (s, 3 H), 3.58 (d, | | 50/25/25/0.1 | | |
| | J = 12.4 Hz, 1 H), 3.46 (s, | | Flow Rate-1.0 | | |
| | 3 H), 3.38-3.32 (m, 1 H), | | ml/min, | | |
| | 3.13-3.07 (m, 1 H), 2.43 (s, | | solubility: | | |
| | 3 H), 0.81-0.60 (m, 4 H). | | MeOH, Rt = | | |
| Ex. 173B | ¹H-NMR (400 MHZ, DMSO-d6): | Condition K: | 5.40 min for | Yellow | 39.1 |
| (Peak 2) | δ 7.69 (s, 1 H), 7.57- | Rt = 3.31 | Peak 1 and Rt = | solid | |
| | 7.52 (m, 2 H), 7.41-7.39 (m, | min. m/z | 7.27 min for | | |
| | 2 H), 4.79 (d, J = 10.8 Hz, | 513.2 [M + H]⁺. | Peak 2. | | |
| | 1 H), 3.99 (d, J = 13.6 Hz, | | | | |
| | 1 H), 3.78 (s, 3 H), 3.58 (d, | | | | |
| | J = 12.4 Hz, 1 H), 3.46 (s, | | | | |
| | 3 H), 3.38-3.32 (m, 1 H), | | | | |
| | 3.13-3.07 (m, 1 H), 2.43 (s, | | | | |
| | 3 H), 0.81-0.60 (m, 4 H). | | | | |

### Example 174

### Example 174A: Enantiomer Peak 1

### (R)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one

### Example 174B: Enantiomer Peak 2

### (S)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one

### Step-1—Preparation of 8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one

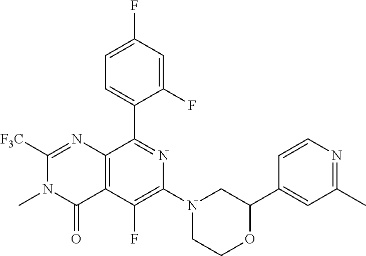

To a stirred solution of 6-chloro-8-(2,4-difluorophenyl)-5-fluoro-3-methyl-2-(trifluoromethyl)pyrido[3,4-d]pyrimidin-4 (3H)-one (100 mg, 0.25 mmol, Examples 158, Step-3) in toluene (10 mL) were added subsequently 2-(2-methylpyridin-4-yl)morpholine (58 mg, 0.33 mmol), Caesium carbonate (165 mg, 0.51 mmol) and xanthphos (14 mg, 0.025 mmol). The resulting mixture was degassed with argon and added Pd2dba3 (23 mg 0.056 mmol). The resulting mixture was heated at 70° C. for 4 h. After completion, reaction mixture was passed through a pad of celite and washed with DCM and concentrated. Crude product was purified by reverse phase preparative HPLC to 8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one (50.0 mg, 40%) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 45% A and 55% B, then 45% A and 55% B in 3 min, then to 25% A and 75% B in 20 min., then to 5% A and 95% B in 21 min., held this composition up to 22 min. for column washing, then returned to initial composition in 23 min. and held till 25 min.

1H NMR (400 MHz, DMSO-d6) δ 8.43 (d, J=5.2 Hz, 1H), 7.69-7.63 (m, 1H), 7.41-7.435 (m, 1H), 7.30 (s, 1H), 7.25-7.21 (m, 2H), 4.74-4.71 (dd, J=10.4 Hz, J=2.0 Hz, 1H), 4.17-4.05 (m, 3H), 3.89-3.83 (m, 1H), 3.52 (s, 3H), 3.26-3.22 (m, 1H), 3.03-2.97 (m, 1H), 2.51 (s, 3H).

LCMS Condition K: Rt=2.82 min. m/z 536.5 [M+H]⁺.

### Step-2—Preparation of (R)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one [Example 174A] and (S)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one [Example 174B]

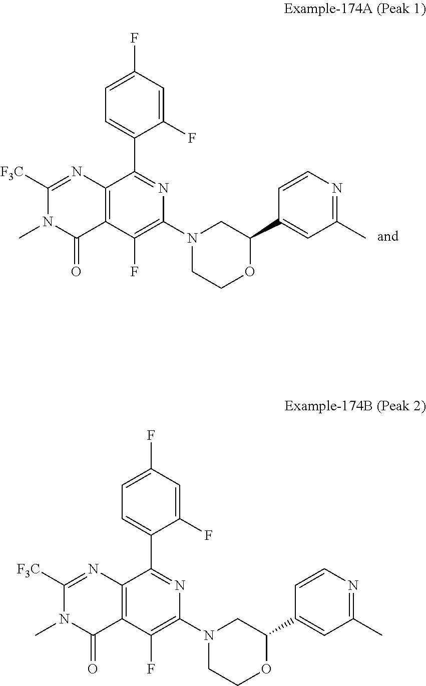 [SMILES: Cc1cc([C@@H]2CN(c3nc(-c4ccc(F)cc4F)c4nc(C(F)(F)F)n(C)c(=O)c4c3F)CCO2)ccn1 ; Cc1cc([C@H]2CN(c3nc(-c4ccc(F)cc4F)c4nc(C(F)(F)F)n(C)c(=O)c4c3F)CCO2)ccn1]

Chiral separation of 8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one (50 mg) was done by normal phase chiral prep HPLC to afford Example 174A, (R)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one (19 mg, 37.8%; eluted first, assigned as Peak 1 with arbitrary assignment of stereochemistry) as yellow solid and Example 174B, (S)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one (20 mg, 39.2% yield, eluted later, assigned as Peak 2 with arbitrary assignment of stereochemistry) as yellow solid.

Chiral separation was done on Agilent 1200 series instrument. Column name: CHIRALPAK IG (250×30 mm) 5u. Operating at ambient temperature and flow rate is 27.0 mL/min. Mobile phase was mixture of 70% Hexane, 15% Ethyl alcohol and 15% DCM, held this isocratic mixture run up-to 25 min with wavelength of 330 nm

Example 174A (Peak 1): 1H NMR (400 MHz, DMSO-d6) δ 8.43 (d, J=5.2 Hz, 1H), 7.69-7.63 (m, 1H), 7.41-7.435 (m, 1H), 7.30 (s, 1H), 7.25-7.21 (m, 2H), 4.74-4.71 (dd, J=10.4 Hz, J=2.0 Hz, 1H), 4.17-4.05 (m, 3H), 3.89-3.83 (m, 1H), 3.52 (s, 3H), 3.26-3.22 (m, 1H), 3.03-2.97 (m, 1H), 2.51 (s, 3H).

LCMS Condition K: Rt=2.82 min. m/z 536.5 [M+H]⁺.

Example 174B (Peak 2): 1H NMR (400 MHz, DMSO-d6) δ 8.43 (d, J=5.2 Hz, 1H), 7.69-7.63 (m, 1H), 7.41-7.435 (m, 1H), 7.30 (s, 1H), 7.25-7.21 (m, 2H), 4.74-4.71 (dd, J=10.4 Hz, J=2.0 Hz, 1H), 4.17-4.05 (m, 3H), 3.89-3.83 (m, 1H), 3.52 (s, 3H), 3.26-3.22 (m, 1H), 3.03-2.97 (m, 1H), 2.51 (s, 3H).

LCMS Condition K: Rt=2.82 min. m/z 536.5 [M+H]⁺.

Analytical chiral HPLC: CHIRALPAK IG (250×4.6) mm, 5μ M.P.—Hexane/DCM/EtOH/IPAmine: 50/25/25/0.1 Flow Rate—1.0 ml/min, solubility: MeOH, Rt=5.00 min for Peak 1 and Rt=6.22 min for Peak 2.

### Example 175 and Example 176

### Example 175A: Enantiomer Peak 1

### 8-(2,4-difluorophenyl)-2,3-dimethyl-6-((2S,6R)-2-methyl-6-(2-methylpyridin-4-yl)morpholino)pyrimido[5,4-d]pyrimidin-4 (3H)-one

### Example 175B: Enantiomer Peak 2

### (8-(2,4-difluorophenyl)-2,3-dimethyl-6-((2R,6S)-2-methyl-6-(2-methylpyridin-4-yl)morpholino)pyrimido[5,4-d]pyrimidin-4 (3H)-one

### Example 176A: Enantiomer Peak 1

### 8-(4-chloro-2-fluorophenyl)-2,3-dimethyl-6-((2S,6R)-2-methyl-6-(2-methylpyridin-4-yl)morpholino)pyrimido[5,4-d]pyrimidin-4 (3H)-one

### Example 176B: Enantiomer Peak 2

### 8-(4-chloro-2-fluorophenyl)-2,3-dimethyl-6-((2R,6S)-2-methyl-6-(2-methylpyridin-4-yl)morpholino)pyrimido[5,4-d]pyrimidin-4 (3H)-one

### Step-1—Preparation of 4-(1-ethoxyvinyl)-2-methylpyridine

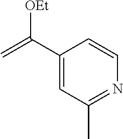

To a stirred solution of 4-bromo-2-methylpyridine (20 g, 116.13 mmol) in toluene (200 mL) was added tributyl(1-ethoxyvinyl)stannane (45 mL) Resulting mixture was degassed with argon and Pd(PPh₃)₄ (6.71 g, 5.81 mmol) was added under inert atmosphere. Resulting mixture was heated to 110° C. for 16 h. Reaction mixture was filtered through a short pad of celite and washed with ethyl acetate. Combined filtrate was concentrated under reduced pressure to afford 4-(1-ethoxyvinyl)-2-methylpyridine (20.2 g, crude) as black liquid. The crude material was used in the next step without further purification.

LCMS Condition K: Rt=0.72 min. m/z 164.0 [M+H]⁺.

### Step-2—Preparation of 1-(2-methylpyridin-4-yl)ethan-1-one

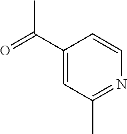

To a stirred material of 4-(1-ethoxyvinyl)-2-methylpyridine (20.2 g, crude) 0° C. was added dil. HCl (25 mL, 3N) and the resulting solution was stirred at room temperature for 2 h. Reaction mixture was quenched with sodium bicarbonate solution and extracted with ethyl acetate. The combined organic layers were washed with water, brine, dried over anhydrous sodium sulphate, filtered and concentrated under reduced pressure. Crude mass was purified by column chromatography (silica gel; 10-20% ethyl acetate-hexane) to afford 1-(2-methylpyridin-4-yl)ethan-1-one (10.1 g, 63% yield two step) as colorless liquid.

LCMS Condition F: Rt=0.33 min. m/z 136.2 [M+H]⁺.

### Step-3—Preparation of 2-bromo-1-(2-methylpyridin-4-yl)ethan-1-one

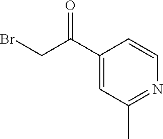

To a stirred solution of 1-(2-methylpyridin-4-yl)ethan-1-one (8 g, 59.25 mmol) in HBr (50 mL; 33% in glacial acetic acid) was added bromine (3.1 mL, 59.25 mmol) dropwise. Resulting mixture was heated at 80° C. for 1 h. The reaction mixture cooled to room temperature and poured into the ether and it was stirred for another 30 min. Off-white solid precipitated out, filtered and dried under vacuum to afford 2-bromo-1-(2-methylpyridin-4-yl)ethan-1-one (9 g, 70% yield) as white solid.

LCMS Condition F: Rt=0.27 min. m/z 214.0 [M+H]⁺.

### Step-4—Preparation of 2-(benzyl(2-hydroxypropyl)amino)-1-(2-methylpyridin-4-yl)ethan-1-one

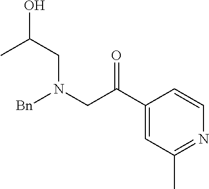 [SMILES: Cc1cc(C(=O)CN(Cc2ccccc2)CC(C)O)ccn1]

To a stirred solution of 2-bromo-1-(2-methylpyridin-4-yl)ethan-1-one (9 g, 42.25 mmol) in THF (100 mL) were added DIPEA (37 mL, 211 mmol) and 1-(benzylamino)propan-2-ol (8.3 g, 50.7 mmol) at room temperature. Resulting mixture was stirred at room temperature for 16 h. Reaction mixture was quenched with water and extracted with ethyl acetate. The combined organic layer was washed with water, brine, dried over anhydrous sodium sulphate, filtered and concentrated under reduced pressure. Crude mass was purified by combi-flash chromatography (silica gel; 50-80% ethyl acetate-hexane) to afford 2-(benzyl(2-hydroxypropyl)amino)-1-(2-methylpyridin-4-yl)ethan-1-one (9 g, 71% yield) as yellow liquid.

LCMS Condition F: Rt=1.51 min. m/z 300.4 [M+H]⁺.

### Step-5—Preparation of 4-benzyl-2-methyl-6-(2-methylpyridin-4-yl)-3,4-dihydro-2H-1,4-oxazine

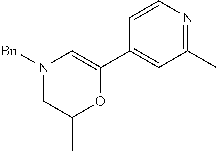 [SMILES: Cc1cc(C2=CN(Cc3ccccc3)CC(C)O2)ccn1]

To a stirred solution of 2-(benzyl(2-hydroxypropyl)amino)-1-(2-methylpyridin-4-yl)ethan-1-one (2.5 g, 83.50 mmol) in dichloroethane (100 mL) were added TMSOTf (45 mL, 250 mmol) at 0° C. Resulting mixture was heated to 80° C. for 16 h. Reaction mixture was quenched with sodium bicarbonate and extracted with 10% methanol-dichloromethane. The combined organic layer was washed with water, brine, dried over anhydrous sodium sulphate, filtered and concentrated under reduced pressure to afford 4-benzyl-2-methyl-6-(2-methylpyridin-4-yl)-3,4-dihydro-2H-1,4-oxazine (2 g, 85% yield) as yellow liquid.

LCMS Condition F: Rt=1.42 min. m/z 282.0 [M+H]⁺.

### Step-6—Preparation of 2-methyl-6-(2-methylpyridin-4-yl)morpholine

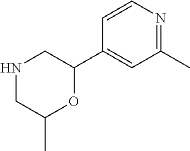

To a stirred solution of 4-benzyl-2-methyl-6-(2-methylpyridin-4-yl)-3,4-dihydro-2H-1,4-oxazine (1.0 g, 3.56 mmol) in methanol (20 mL) were added ammonium formate (1.1 g, 17.82 mmol) and Pd(OH)₂ (1 g, 20% on carbon) under nitrogen atmosphere. Resulting mixture was heated to 80° C. for 16 h. Reaction mixture was filtered through a pad of celite bed, washed with methanol and concentrated. The crude residue was quenched with sodium bicarbonate solution, extracted with 10% methanol in dichloromethane mixture, dried over sodium sulphate and concentrated under reduced pressure. Crude mass was purified by combi-flash chromatography (silica gel; 10% methanol-dichloromethane) to afford 2-methyl-6-(2-methylpyridin-4-yl)morpholine (500 mg, 73.0% yield) as a gum.

LCMS Condition F: Rt=0.25 min m/z 193.2 [M+H]⁺.

Example 175[A-B] to Example 176[A-B]were then synthesized using similar procedures as Examples 1 and 112.

Chiral separation details of Example 176A-176B to 177A-177B are captured in following table:

| Comp-ound No. | | Starting | Chiral separation |
|---|---|---|---|
| Structure | Name | material | specification |
| Ex. 175A (Peak 1) | 8-(2,4- | 8-(2,4- | Similar |
| | difluorophenyl)-2,3- | difluorophenyl)- | specifications as |
| 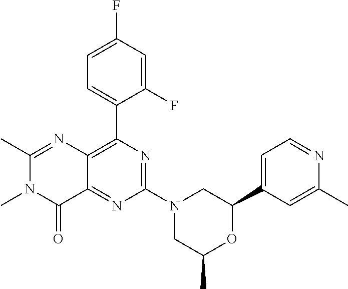 | dimethyl-6-((2S,6R)- 2-methyl-6-(2- methylpyridin-4- yl)morpholino)pyrimi do[5,4-d]pyrimidin- 4(3H)-one | 2,3-dimethyl- 6-(2-methyl-6- (2- methylpyridin- 4- yl)morpholino) pyrimido[5,4- d]pyrimidin- 4(3H)-one | mentioned in example-100. Peak 1 Absolute stereochemistry unknown |
| Ex. 175B (Peak 2) | 8-(2,4- | | Similar |
| | difluorophenyl)-2,3- | | specifications as |
| 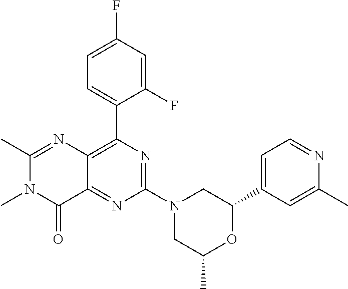 | dimethyl-6-((2R,6S)- 2-methyl-6-(2- methylpyridin-4- yl)morpholino) pyrimido[5,4-d] pyrimidin- 4(3H)-one | | mentioned in example-100. Peak 2 Absolute stereochemistry unknown |
| Ex. 176A (Peak 1) | 8-(4-chloro-2- | 8-(4-chloro-2- | Similar |
| | fluorophenyl)-2,3- | fluorophenyl)- | specifications as |
| 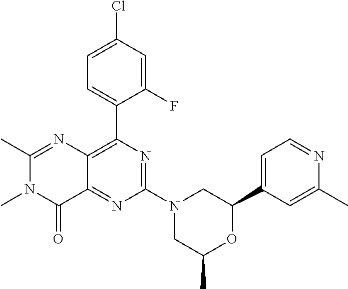 | dimethyl-6-((2S,6R)- 2-methyl-6-(2- methylpyridin-4- yl)morpholino) pyrimido[5,4-d] pyrimidin- 4(3H)-one | 2,3-dimethyl-6- (2-methyl-6-(2- methylpyridin- 4- yl)morpholino) pyrimido[5,4- d]pyrimidin- 4(3H)-one | mentioned in example-100. Peak 1 Absolute stereochemistry unknown |
| Ex. 176B (Peak 2) | 8-(4-chloro-2- | | Peak 2 |
| | fluorophenyl)-2,3- | | Absolute |
| 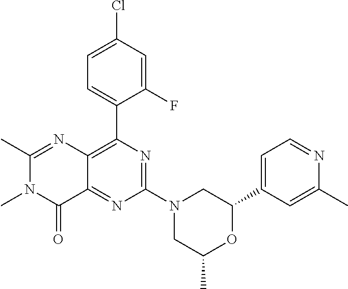 | dimethyl-6-((2R,6S)- 2-methyl-6-(2- methylpyridin-4- yl)morpholino) pyrimido[5,4-d] pyrimidin- 4(3H)-one | | stereochemistry unknown |

Following table describes analytical data analysis and yield information of examples 175A-175B to examples 176A-177B:

| | | | Analytical | | Yield |
|---|---|---|---|---|---|
| Compound No. | HNMR | LCMS | chiral HPLC | Description | (%) |
| Ex. 175A | ¹H-NMR (400 MHZ, CDCI3): | Condition K: | CHIRALPAK IG | Yellow | 41.5 |
| (Peak 1) | δ 8.50 (d, J = 5.2 Hz, 1 H), | Rt = 2.64 | (250 × 4.6) mm, | solid | |
| | 7.68-7.63 (m, 1 H), 7.42- | min. m/z | 5 μ M.P.- | | |
| | 7.39 (m, 2 H), 7.03-6.91 (m, | 495.4 [M + H]⁺. | Hexane/DCM/ | | |
| | 2 H), 5.02 (d, J = 12.4 Hz, | | EtOH/IPAmine- | | |
| | 1 H), 4.87 (d, J = 10.8 Hz, | | 50/25/25/0.1 | | |
| | 1 H), 4.63 (d, J = 9.6 Hz, 1 H), | | Flow Rate-1.0 | | |
| | 3.83 (d, J = 6.4 Hz, 1 H), 3.62 | | ml/min Solubility: | | |
| | (s, 3 H), 2.89-2.79(m, 2 H), | | MeOH, Rt = | | |
| | 2.71 (s, 3 H), 2.51 (s, 3 H), | | 5.58 min for | | |
| | 1.36 (d, J = 6.0 Hz, 3 H). | | Peak 1 and Rt = | | |
| Ex. 175B | ¹H-NMR (400 MHZ, CDCI3): | Condition K: | 7.62 min for | Yellow | 40.2 |
| (Peak 2) | δ 8.50 (d, J = 5.2 Hz, 1 H), | Rt = 2.64 | Peak 2. | solid | |
| | 7.68-7.63 (m, 1 H), 7.42- | min. m/z | | | |
| | 7.39 (m, 2 H), 7.03-6.91 (m, | 495.4 [M + H]⁺. | | | |
| | 2 H), 5.02 (d, J = 12.4 Hz, | | | | |
| | 1 H), 4.87 (d, J = 10.8 Hz, | | | | |
| | 1 H), 4.63 (d, J = 9.6 Hz, 1 H), | | | | |
| | 3.83 (d, J = 6.4 Hz, 1 H), 3.62 | | | | |
| | (s, 3 H), 2.89-2.79(m, 2 H), | | | | |
| | 2.71 (s, 3 H), 2.51 (s, 3 H), | | | | |
| | 1.36 (d, J = 6.0 Hz, 3 H). | | | | |
| Ex. 176A | ¹H NMR (400 MHZ, DMSO-d₆): | Condition K: | CHIRALPAK IG | Yellow | 39.1 |
| (Peak 1) | δ 8.44 (d, J = 4.8 Hz, | Rt = 2.75 | (250 × 4.6) mm, | solid | |
| | 1 H), 7.67(t, J = 8.0 Hz, 1 H), | min. m/z | 5 μ M.P.- | | |
| | 7.63-7.60 (m, 1 H), 7.47- | 500.4 [M + H]⁺. | Hexane/DCM/ | | |
| | 7.7.45 (dd, J = 8.4 Hz, J = 1.2 | | EtOH/IPAmine- | | |
| | Hz, 1 H), 7.30 (s, 1 H), 7.22 | | 50/25/25/0.1 | | |
| | (d, J = 4.8 Hz, 1 H), 4.74-4.64 | | Flow Rate-1.0 | | |
| | (m, 3 H), 3.82 (bs, 1 H), 3.51 | | ml/min: | | |
| | (s, 3 H), 2.88-2.76 (m, 2 H), | | Solubility: | | |
| | 2.49 (s, 3 H), 2.44 (s, 3 H), | | MeOH, Rt = | | |
| | 1.28 (d, J = 6.0 Hz, 3 H). . | | 5.66 min for | | |
| Ex. 176B | ¹H NMR (400 MHZ, DMSO-d₆): | Condition K: | Peak 1 and Rt = | Yellow | 38.6 |
| (Peak 2) | δ 8.44 (d, J = 4.8 Hz, | Rt = 2.75 | 6.76 min for | solid | |
| | 1 H), 7.67(t, J = 8.0 Hz, 1 H), | min. m/z | Peak 2. | | |
| | 7.63-7.60 (m, 1 H), 7.47- | 500.4 [M + H]⁺. | | | |
| | 7.7.45 (dd, J = 8.4 Hz, J = 1.2 | | | | |
| | Hz, 1 H), 7.30 (s, 1 H), 7.22 | | | | |
| | (d, J = 4.8 Hz, 1 H), 4.74-4.64 | | | | |
| | (m, 3 H), 3.82 (bs, 1 H), 3.51 | | | | |
| | (s, 3 H), 2.88-2.76 (m, 2 H), | | | | |
| | 2.49 (s, 3 H), 2.44 (s, 3 H), | | | | |
| | 1.28 (d, J = 6.0 Hz, 3 H). | | | | |

### Example 177

### Example 177A: Enantiomer Peak 1

### 6-((2R,6S)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one

### Example 177B: Enantiomer Peak 2

### 6-((2S,6R)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one

### Example 177C

### 6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (trans-racemate)

### Step-1—Preparation of 4-bromo-1-cyclopropyl-1H-pyrazole

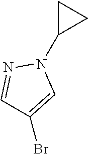

To a stirred solution of 4-bromo-1H-pyrazole (10 g, 68.02 mmol) in dichloroethane (200 mL) were added cyclopropyl boronic acid (11.7 g, 137.93 mmol), 2,2-bipyridyl (11.7 g, 74.83 mmol) and sodium carbonate (15.9 g, 149.66 mmol) under oxygen atmosphere. Finally, cupper acetate (13.6 g, 74.83 mmol) was added to the reaction mixture. Resulting mixture was heated to 65° C. for 16 h. Reaction mixture was filtered through a pad of celite bed, washed with methanol and concentrated under reduced pressure. The crude residue was extracted with ethyl acetate, washed with brine, dried over sodium sulphate and concentrated. Crude product was purified by combi-flash chromatography (silica gel; 10% ethyl acetate-hexane) to afford 4-bromo-1-cyclopropyl-1H-pyrazole (6.5 g, 51% yield) as colourless liquid.

LCMS Condition C: Rt=1.86 min. m/z 187.1 [M+H]⁺.

### Step-2—Preparation of 1-cyclopropyl-4-(1-ethoxyvinyl)-1H-pyrazole

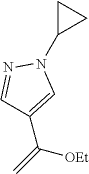

To a stirred solution of 4-bromo-1-cyclopropyl-1H-pyrazole (5 g, 26.73 mmol) in toluene (100 mL) was added tributyl(1-ethoxyvinyl)stannane (9.9 mL, 29.42 mmol). Resulting mixture was degassed with argon and Pd(PPh₃)₄ (1.54 g, 1.33 mmol) was added under inert atmosphere. Resulting mixture was heated to 110° C. for 16 h. Reaction mixture was filtered through a short pad of celite and washed with ethyl acetate. Combined layer was concentrated to get 1-cyclopropyl-4-(1-ethoxyvinyl)-1H-pyrazole (4.5 g, crude). The crude product was carried forward for the next step without further purification.

### Step-3—Preparation of 2-bromo-1-(1-cyclopropyl-1H-pyrazol-4-yl)ethan-1-one

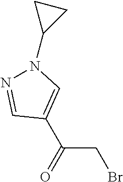 [SMILES: O=C(CBr)c1cnn(C2CC2)c1]

To a stirred solution of 1-cyclopropyl-4-(1-ethoxyvinyl)-1H-pyrazole (4.5 g, crude) in THF (120 mL) and water (50 mL) at 0° C. was added NBS (10 g, 62.5 mmol). Resulting reaction mixture was stirred at room temperature for 3 h. Reaction mixture was extracted with ethyl acetate, washed brine, dried over anhydrous sodium sulphate, filtered and concentrated under reduced pressure. Crude mass was purified by column chromatography (silica gel; 25-40% ethyl acetate-hexane) to afford 2-bromo-1-(1-cyclopropyl-1H-pyrazol-4-yl)ethan-1-one (1.5 g, 23.4% yield) as white solid.

LCMS Condition C: Rt=1.74 min. m/z 229.1 [M+H]⁺.

### Step-4—Preparation of 2-(benzyl(2-hydroxypropyl)amino)-1-(1-cyclopropyl-1H-pyrazol-4-yl)ethan-1-one

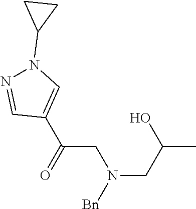 [SMILES: CC(O)CN(CC(=O)c1cnn(C2CC2)c1)Cc1ccccc1]

To a stirred solution of 2-bromo-1-(1-cyclopropyl-1H-pyrazol-4-yl)ethan-1-one (1.5 g, 6.55 mmol) in ACN (30 mL) were added K₂CO₃ (1.8 g, 13.0 mmol) and 1-(benzylamino)propan-2-ol (1.18 g, 7.20 mmol) at room temperature. Resulting reaction mixture was stirred at room temperature for 6 h. Reaction mixture was quenched with water and extracted with ethyl acetate. The combined organic layer was washed with water, brine, dried over anhydrous sodium sulphate, filtered and concentrated under reduced pressure. Crude mass was purified by combi-flash chromatography (silica gel, 50-80% ethyl acetate-hexane) to afford 2-(benzyl(2-hydroxypropyl)amino)-1-(1-cyclopropyl-1H-pyrazol-4-yl)ethan-1-one (1.7 g, 82% yield) as yellow gum.

LCMS Condition G: Rt=4.35 min. m/z 314.1 [M+H]⁺.

### Step-5—Preparation of 1-(benzyl(2-(1-cyclopropyl-1H-pyrazol-4-yl)-2-hydroxyethyl)amino)propan-2-ol

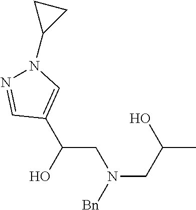 [SMILES: CC(O)CN(Cc1ccccc1)CC(O)c1cnn(C2CC2)c1]

To a stirred solution of 2-(benzyl(2-hydroxypropyl)amino)-1-(1-cyclopropyl-1H-pyrazol-4-yl)ethan-1-one (1.7 g, 5.4 mmol) in MeOH (20 mL) at 0° C. was added NaBH₄ (310 mg, 8.1 mmol) was added portion wise. The resulting mixture was stirred at room temperature for 1 h. Reaction mixture was quenched with ice cold water and extracted with dichloromethane. The combined organic layers were washed with brine, dried over anhydrous sodium sulphate, filtered and concentrated under reduced pressure.

Crude mass was purified by column chromatography (silica-gel; 10-20% methanol-dichloromethane) to afford 1-(benzyl(2-(1-cyclopropyl-1H-pyrazol-4-yl)-2-hydroxyethyl)amino)propan-2-ol (1.5 g, 88% yield) as yellow gum.

LCMS Condition K: Rt=1.37 min. m/z 316.3 [M+H]⁺.

### Step-6—Preparation of 4-benzyl-2-(1-cyclopropyl-1H-pyrazol-4-yl)-6-methylmorpholine

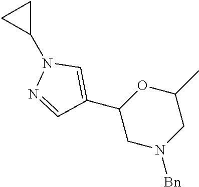 [SMILES: CC1CN(Cc2ccccc2)CC(c2cnn(C3CC3)c2)O1]

To a stirred solution of 1-(benzyl(2-(1-cyclopropyl-1H-pyrazol-4-yl)-2-hydroxyethyl)amino)propan-2-ol (1.5 g, 4.7 mmol) in 1,4-dioxane (5 mL) was added 4 M HCl-1,4-Dioxane solution (5 mL) at room temperature. Resulting mixture was heated at 110° C. for 4 h. The reaction mixture cooled to room temperature and poured into the ice cold water, neutralised with sodium bicarbonate, extracted with 10% methanol-dichloromethane, dried over sodium sulphate and concentrated to afford 4-benzyl-2-(1-cyclopropyl-1H-pyrazol-4-yl)-6-methylmorpholine (1.0 g, 71% yield) as a gum.

LCMS Condition K: Rt=2.09 min. m/z 298.3 [M+H]⁺.

### Step-6—Preparation of 2-(1-cyclopropyl-1H-pyrazol-4-yl)-6-methylmorpholine

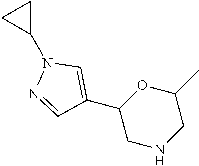

To a stirred solution of 4-benzyl-2-(1-cyclopropyl-1H-pyrazol-4-yl)-6-methylmorpholine (1 g, 3.35 mmol) in methanol (20 mL) were added ammonium formate (1.1 g, 17.82 mmol) and Pd(OH)₂ (300 mg, 20% on carbon) under nitrogen atmosphere. Resulting mixture was heated to 80° C. for 1 h. Reaction mixture was filtered through a pad of celite bed, washed with methanol and concentrated. The crude residue was quenched with sodium bicarbonate solution, extracted with 10% methanol/dichloromethane mixture, dried over sodium sulphate and concentrated. Crude mass was purified by combi flash chromatography (silica gel: 10% methanol-dichloromethane) to afford 2-(1-cyclopropyl-1H-pyrazol-4-yl)-6-methylmorpholine (500 mg, 71% yield) as gum.

Chiral SFC data showed that formation of cis/trans ratio (3:1) and which was further confirmed by 2D NMR in Example 178A.

LCMS Condition F: Rt=0.25 min. m/z 207.2 [M+H]⁺.

### Step-7—Preparation of 6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (Cis racemate and trans-racemate)

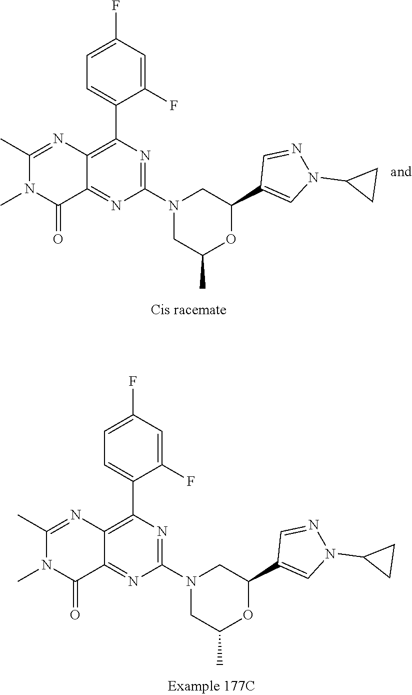 [SMILES: Cc1nc2c(-c3ccc(F)cc3F)nc(N3C[C@@H](c4cnn(C5CC5)c4)O[C@@H](C)C3)nc2c(=O)n1C ; Cc1nc2c(-c3ccc(F)cc3F)nc(N3C[C@@H](C)O[C@H](c4cnn(C5CC5)c4)C3)nc2c(=O)n1C]

To a stirred solution of 6-chloro-8-(2,4-difluorophenyl)-2,3-dimethylpyrimido[5,4-d]pyrimidin-4 (3H)-one (400 mg, 1.24 mmol) in DMSO (5.0 mL) were added 2-(1-cyclopropyl-1H-pyrazol-4-yl)-6-methylmorpholine (385 mg, 1.86 mmol) and DIPEA (0.7 mL, 3.72 mmol). The resulting mixture was heated at 100° C. for 16 h. After completion, reaction mixture was passed through a pad of celite and washed with DCM and concentrated. Crude product was purified by reverse phase preparative HPLC to have two isomeric compound (3:1) 6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (cis-racemate, 115 mg, 19% yield) as yellow solid and 6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (trans-racemate, 45 mg, 7% yield) as yellow solid.

Preparative HPLC was done on Waters auto purification instrument. Column name: Hydrosphare C18 (250×20 mm, 5μ) operating at ambient temperature and flow rate of 16 mL/min. Mobile phase: A=10 mM Ammonium Acetate in water, B=Acetonitrile; Gradient Profile: Mobile phase initial composition of 60% A and 40% B, then 45% A and 55% B in 3 min, then to 30% A and 70% B in 20 min., then to 5% A and 95% B in 21 min., held this composition up to 22 min. for column washing, then returned to initial composition in 23 min. and held till 25 min.

6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (cis-racemate):

1H NMR (400 MHz, CDCl3) δ 7.67-7.61 (m, 1H), 7.53 (d, J=6.0 Hz, 2H), 7.00 (t, J=7.6 Hz, 1H), 6.92 (t, J=9.6 Hz, 1H), 4.91 (d, J=12.0 Hz, 1H), 4.81 (d, J=12.8 Hz, 1H), 4.56 (d, J=10.0 Hz, 1H), 3.79 (bs, 1H), 3.61-3.57 (s, 4H), 3.01 (t, J=11.6 Hz, 1H), 2.79 (t, J=11.6 Hz, 1H), 2.50 (s, 3H), 1.30 (d, J=5.6 Hz, 3H), 1.11-1.00 (m, 2H), 0.86-0.85 (m, 2H).

LCMS Condition K: Rt=2.89 min. m/z 494.4 [M+H]⁺.

### Example 178C: 6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (trans-racemate)

1H NMR (400 MHz, CDCl3) δ 7.69-7.63 (m, 1H), 7.50 (brs, 2H), 7.00 (t, J=7.6 Hz, 1H), 6.94 (t, J=9.6 Hz, 1H), 5.03 (brs, 1H), 4.70 (d, J=13.6 Hz, 1H), 4.48-4.46 (m, 1H), 3.89-3.87 (m, 1H), 3.75 (brs, 1H), 3.61 (s, 3H), 3.43-3.41 (m, 1H), 3.19 (brs, 1H), 2.50 (s, 3H), 1.22 (d, J=6.4 Hz, 3H), 1.11-0.94 (m, 2H), 0.86-0.85 (m, 2H).

LCMS Condition K: Rt=2.82 min. m/z 494.4 [M+H]⁺.

### Step-8—Preparation of 6-((2R,6S)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (Example 178A] and 6-((2S,6R)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one [Example 178B]

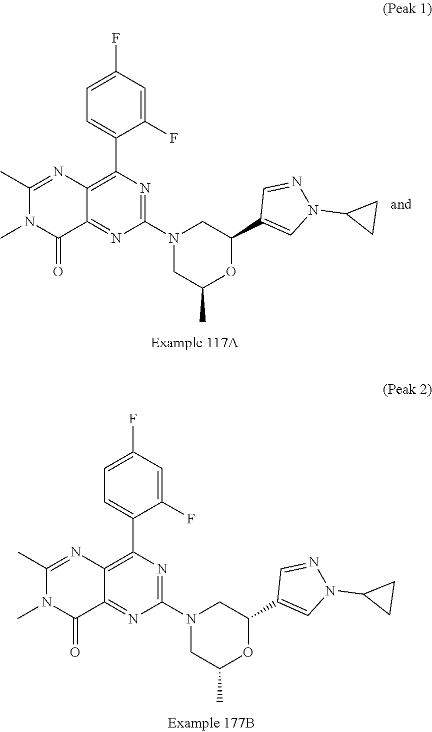 [SMILES: Cc1nc2c(-c3ccc(F)cc3F)nc(N3C[C@@H](c4cnn(C5CC5)c4)O[C@@H](C)C3)nc2c(=O)n1C ; Cc1nc2c(-c3ccc(F)cc3F)nc(N3C[C@@H](C)O[C@@H](c4cnn(C5CC5)c4)C3)nc2c(=O)n1C]

Chiral separation of 6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (cis-racemate, 115 mg) was done by SFC afforded Example 178A, 6-((2R,6S)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (29 mg, 25%; eluted first, assigned as Peak 1 with arbitrary assignment of stereochemistry) as yellow solid and Example 178B, 6-((2S,6R)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (24 mg, 23% yield, eluted later, assigned as Peak 2 with arbitrary assignment of stereochemistry) as yellow solid.

SFC Prep Purification of the sample was run on Pic Solution 175 instrument equipped with Knauer UV Detector 40D by using I CELLULOSE-Z (21.1 mm×250 mm). 5p Column operating at 35° C. temperature, maintaining flow rate of 70 mL/min, using 80% CO2 in super critical state & 20% of 100% Methanol as Mobile phase, Run this isocratic mixture up to 20.0 minutes and also maintained the isobaric condition of 100 bar at 220 nm wavelength.

Example 177A (Peak 1): 1H NMR (400 MHz, CDCl3) δ 7.67-7.61 (m, 1H), 7.53 (d, J=6.0 Hz, 2H), 7.00 (t, J=7.6 Hz, 1H), 6.92 (t, J=9.6 Hz, 1H), 4.91 (d, J=12.0 Hz, 1H), 4.81 (d, J=12.8 Hz, 1H), 4.56 (d, J=10.0 Hz, 1H), 3.79 (bs, 1H), 3.61-3.57 (m, 4H), 3.01 (t, J=11.6 Hz, 1H), 2.79 (t, J=11.6 Hz, 1H), 2.50 (s, 3H), 1.30 (d, J=5.6 Hz, 3H), 1.11-1.00 (m, 2H), 0.86-0.85 (m, 2H).

LCMS Condition K: Rt=2.89 min. m/z 494.4 [M+H]⁺.

Example 177B (Peak 2): 1H NMR (400 MHz, CDCl3) δ 7.67-7.61 (m, 1H), 7.53 (d, J=6.0 Hz, 2H), 7.00 (t, J=7.6 Hz, 1H), 6.92 (t, J=9.6 Hz, 1H), 4.91 (d, J=12.0 Hz, 1H), 4.81 (d, J=12.8 Hz, 1H), 4.56 (d, J=10.0 Hz, 1H), 3.79 (bs, 1H), 3.61-3.57 (m, 4H), 3.01 (t, J=11.6 Hz, 1H), 2.79 (t, J=11.6 Hz, 1H), 2.50 (s, 3H), 1.30 (d, J=5.6 Hz, 3H), 1.11-1.00 (m, 2H), 0.86-0.85 (m, 2H).

LCMS Condition K: Rt=2.89 min. m/z 494.4 [M+H]⁺.

Analytical chiral HPLC: I-CELLULOSE Z (150×4.6) mm, 3μ M.P.—0.3% IPamine in MeOH: Flow Rate—4.0 ml/min Solubility: MeOH, Rt=1.81 min for Peak 1 and Rt=1.97 min for Peak 2.

### Example 178 to Example 184

Example 178[A-B] to Example 184[A-B]were synthesized as similar procedure as Example 177.

Chiral separation details of Example 178A-179B to 184A-184B are captured in following table:

| Comp-ound No. | | Starting | Chiral separation |
|---|---|---|---|
| Structure | Name | material | specification |
| Ex. 178A (Peak 1) | 8-(4-chloro-2- | 8-(4-chloro-2- | Similar |
| | fluorophenyl)-6- | fluorophenyl)-6- | specifications as |
| 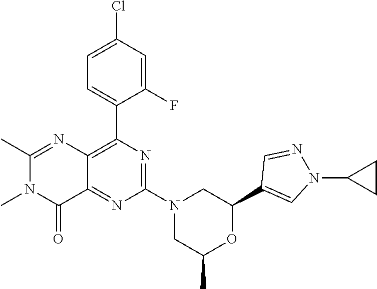 [SMILES: Cc1nc2c(-c3ccc(Cl)cc3F)nc(N3C[C@@H](c4cnn(C5CC5)c4)O[C@@H](C)C3)nc2c(=O)n1C] | ((2R,6S)-2-(1- cyclopropyl-1H- pyrazol-4-yl)-6- methylmorpholino)- 2,3- dimethylpyrimido[5, 4-d]pyrimidin- 4(3H)-one | (2-(1- cyclopropyl-1H- pyrazol-4-yl)-6- methyl- morpholino)-2,3- dimethyl- pyrimido[5,4- d]pyrimidin- 4(3H)-one | mentioned in example-177 Peak 1 Absolute stereochemistry unknown. |
| Ex. 178B (Peak 2) | 8-(4-chloro-2- | | Similar |
| | fluorophenyl)-6- | | specifications as |
| 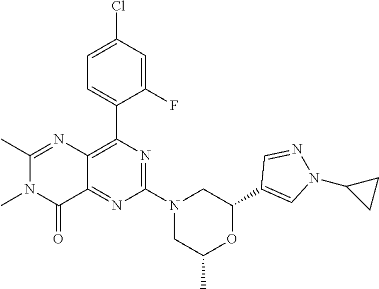 [SMILES: Cc1nc2c(-c3ccc(Cl)cc3F)nc(N3C[C@@H](C)O[C@@H](c4cnn(C5CC5)c4)C3)nc2c(=O)n1C] | ((2S,6R)-2-(1- cyclopropyl-1H- pyrazol-4-yl)-6- methylmorpholino)- 2,3- dimethylpyrimido[5, 4-d]pyrimidin- 4(3H)-one | | mentioned in example-177 Peak 2 Absolute stereochemistry unknown. |
| Ex. 179A (Peak 1) | 8-(2-fluoro-4- | 8-(2-fluoro-4- | Similar |
| | (trifluoromethyl) | (trifluoromethyl) | specifications as |
| 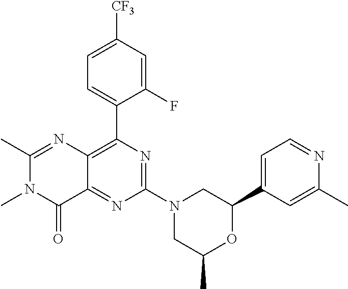 | phenyl)-2,3- dimethyl-6- ((2S,6R)-2-methyl- 6-(2-methylpyridin- 4-yl)morpholino) pyrimido[5,4-d] pyrimidin- 4(3H)-one | phenyl)-2,3- dimethyl-6-(2- methyl-6-(2- methylpyridin-4- yl)morpholino) pyrimido[5,4- d]pyrimidin- 4(3H)-one | mentioned in example-177 Peak 1 Absolute stereochemistry unknown. |
| Ex. 179B (Peak 2) | 8-(2-fluoro-4- | | Peak 2 |
| | (trifluoromethyl) | | Absolute |
| 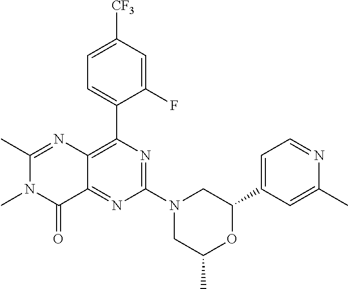 | phenyl)-2,3- dimethyl-6- ((2R,6S)-2-methyl- 6-(2-methylpyridin- 4- yl)morpholino) pyrimido[5,4-d] pyrimidin- 4(3H)-one | | stereochemistry unknown. |
| Ex. 180A (Peak 1) | (R)-8-(6- | 8-(6- | Peak 1 |
| | (difluoromethyl) | (difluoromethyl) | Absolute |
| 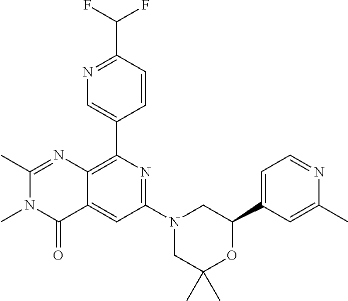 | pyridin-3-yl)-6- (2,2-dimethyl-6-(2- methylpyridin-4- yl)morpholino)-2,3- dimethylpyrido [3,4- d]pyrimidin-4(3H)- one | pyridin-3-yl)-6- (2,2-dimethyl-6- (2-methylpyridin- 4-yl) morpholino)-2,3- dimethylpyrido [3,4-d]pyrimidin- 4(3H)-one | stereochemistry unknown. |
| Ex. 180B (Peak 2) | (S)-8-(6- | | Peak 2 |
| | (difluoromethyl) | | Absolute |
| 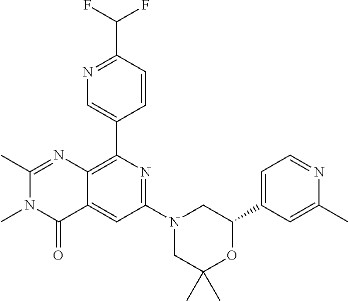 | pyridin-3-yl)-6-(2,2- dimethyl-6-(2- methylpyridin-4- yl)morpholino)-2,3- dimethylpyrido[3,4- d]pyrimidin-4(3H)- one | | stereochemistry unknown. |
| Ex. 181A (Peak 1) | (R)-6-(2,2-difluoro- | 6-(2,2-difluoro- | Peak 1 |
| | 6-(2-methylpyridin- | 6-(2- | Absolute |
| 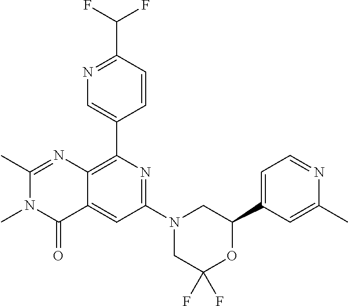 | 4-yl)morpholino)-8- (6- (difluoromethyl) pyridin-3-yl)-2,3- dimethylpyrido[3,4- d]pyrimidin-4(3H)- one | methylpyridin-4- yl)morpholino)- 8-(6- (difluoromethyl) pyridin-3-yl)-2,3- dimethylpyrido [3,4-d] pyrimidin- 4(3H)-one | stereochemistry unknown. |
| Ex. 181B (Peak 2) | (S)-6-(2,2-difluoro- | | Peak 2 |
| | 6-(2-methylpyridin- | | Absolute |
| 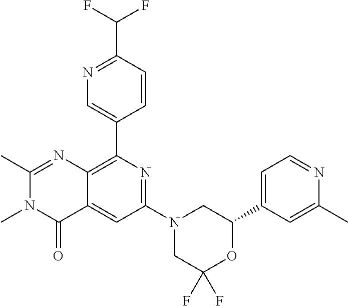 | 4-yl)morpholino)-8- (6-(difluoromethyl) pyridin-3-yl)-2,3- dimethylpyrido[3,4- d]pyrimidin-4(3H)- one | | stereochemistry unknown. |
| Ex. 182A (Peak 1) | 8-(2,4- | 8-(2,4- | Similar |
| | difluorophenyl)-2,3- | difluorophenyl)- | specifications as |
| 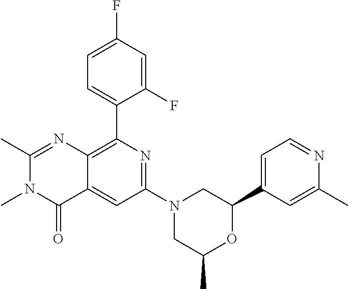 | dimethyl-6- ((2S,6R)-2-methyl- 6-(2-methylpyridin- 4-yl)morpholino) pyrido[3,4-d] pyrimidin- 4(3H)-one | 2,3-dimethyl-6- (2-methyl-6-(2- methylpyridin-4- yl)morpholino) pyrido[3,4- d]pyrimidin- 4(3H)-one | mentioned in example-177 Peak 1 Absolute stereochemistry unknown. |
| Ex. 182B (Peak 2) | 8-(2,4- | | Peak 2 |
| | difluorophenyl)-2,3- | | Absolute |
| 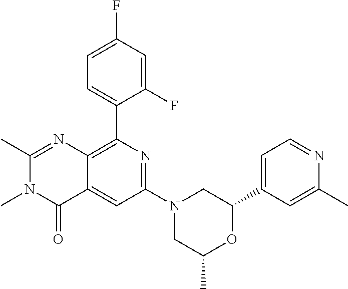 | dimethyl-6- ((2R,6S)-2-methyl- 6-(2-methylpyridin- 4-yl)morpholino) pyrido[3,4-d] pyrimidin- 4(3H)-one | | stereochemistry unknown. |
| Ex. 183A (Peak 1) | 6-((2R,6S)-2-(1- | 6-(2-(1- | Similar |
| | cyclopropyl-1H- | cyclopropyl-1H- | specifications as |
| 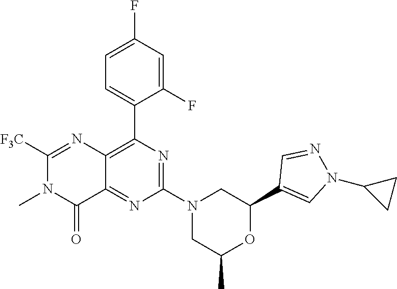 [SMILES: C[C@H]1CN(c2nc(-c3ccc(F)cc3F)c3nc(C(F)(F)F)n(C)c(=O)c3n2)C[C@@H](c2cnn(C3CC3)c2)O1] | pyrazol-4-yl)-6- methylmorpholino)- 8-(2,4- difluorophenyl)-3- methyl-2- (trifluoromethyl) pyrimido[5,4- d]pyrimidin-4(3H)- one | pyrazol-4-yl)-6- methyl- morpholino)- 8-(2,4- difluorophenyl)- 3-methyl-2- (trifluoromethyl) pyrimido[5,4- d]pyrimidin- 4(3H)-one | mentioned in example-177 Peak 1 Absolute stereochemistry unknown |
| Ex. 183B (Peak 2) | 6-((2S,6R)-2-(1- | | Peak 2 |
| | cyclopropyl-1H- | | Absolute |
| 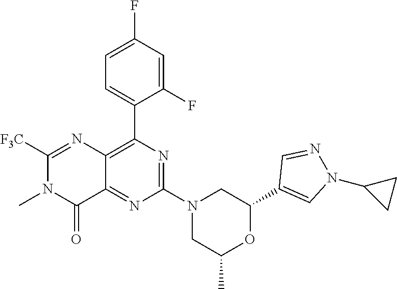 [SMILES: C[C@@H]1CN(c2nc(-c3ccc(F)cc3F)c3nc(C(F)(F)F)n(C)c(=O)c3n2)C[C@H](c2cnn(C3CC3)c2)O1] | pyrazol-4-yl)-6- methylmorpholino)- 8-(2,4- difluorophenyl)-3- methyl-2- (trifluoromethyl) pyrimido[5,4- d]pyrimidin-4(3H)- one | | stereochemistry unknown. |
| Ex. 184A (Peak 1) | 6-((2R,6S)-2-(1- | 6-(2-(1- | Similar |
| | cyclopropyl-1H- | cyclopropyl-1H- | specifications as |
| 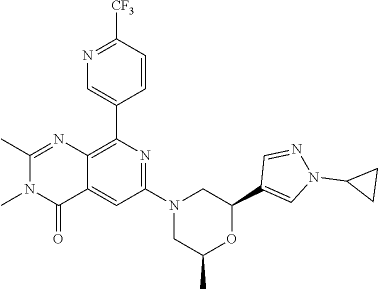 [SMILES: Cc1nc2c(-c3ccc(C(F)(F)F)nc3)nc(N3C[C@@H](c4cnn(C5CC5)c4)O[C@@H](C)C3)cc2c(=O)n1C] | pyrazol-4-yl)-6- methylmorpholino)- 2,3-dimethyl-8-(6- (trifluoromethyl) pyridin-3- yl)pyrido[3,4- d]pyrimidin-4(3H)- one | pyrazol-4-yl)-6- methyl- morpholino)-2,3- dimethyl-8-(6- (trifluoromethyl) pyridin-3- yl)pyrido[3,4- d]pyrimidin- 4(3H)-one | mentioned in example-177 Peak 1 Absolute stereochemistry unknown. |
| Ex. 184B (Peak 2) | 6-((2S,6R)-2-(1- | | Peak 2 |
| | cyclopropyl-1H- | | Absolute |
| 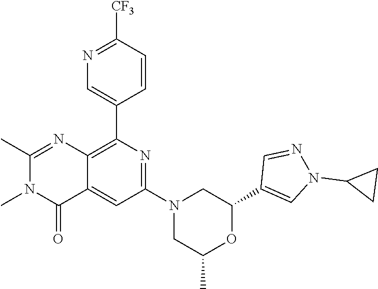 [SMILES: Cc1nc2c(-c3ccc(C(F)(F)F)nc3)nc(N3C[C@@H](C)O[C@@H](c4cnn(C5CC5)c4)C3)cc2c(=O)n1C] | pyrazol-4-yl)-6- methylmorpholino)- 2,3-dimethyl-8-(6- (trifluoromethyl) pyridin-3-yl) pyrido[3,4- d]pyrimidin-4(3H)- one | | stereochemistry unknown. |

Following table describes analytical data analysis and yield information of examples 178A-178B to examples 184A-184B:

| Compound | | | Analytical | | Yield |
|---|---|---|---|---|---|
| No. | HNMR | LCMS | chiral HPLC | Description | (%) |
| Ex. 178A | ¹H-NMR (400 MHz, DMSO-d₆): | Condition | CHIRALCEL OX-H | Yellow | 41.2 |
| (Peak 1) | δ 8.84 (s, 1H), 7.65 (t, J = 8.0 | K: Rt = | (150 × 4.6) mm, | solid | |
| | Hz, 1H), 7.59 (d, J = 9.6 Hz, 1H), | 3.06 min. | 3μ M.P.-0.3% | | |
| | 7.46 (s, 1H), 7.40 (bs, 1H), 4.64 | m/z 510.4 | IPamine in MeOH: | | |
| | (t, J = 12.0 Hz, 2H), 4.50 (d, | [M + H]⁺. | Flow Rate - | | |
| | J = 10.8 Hz, 1H), 3.73-3.68 (m, | | 4.0 ml/min | | |
| | 2H), 3.50 (s, 3H), 3.03 (t, J = | | Solubility: | | |
| | 12.0 Hz, 1H), 2.73 (t, J = 12.0 | | MeOH, Rt = | | |
| | Hz, 1H), 2.43 (s, 3H), 1.96 (d, | | 5.25 min for | | |
| | J = 5.6 Hz, 3H), 1.02-0.97 (m, | | Peak 1 and Rt = | | |
| | 2H), 0.94-0.92 (m, 2H). | | 5.99 min for | | |
| Ex. 178B | ¹H-NMR (400 MHz, DMSO-d₆): | Condition | Peak 2. | Yellow | 39.6 |
| (Peak 2) | δ 8.84 (s, 1H), 7.65 (t, J = 8.0 | K: Rt = | | solid | |
| | Hz, 1H), 7.59 (d, J = 9.6 Hz, 1H), | 3.06 min. | | | |
| | 7.46 (s, 1H), 7.40 (bs, 1H), 4.64 | m/z 510.4 | | | |
| | (t, J = 12.0 Hz, 2H), 4.50 (d, | [M + H]⁺. | | | |
| | J = 10.8 Hz, 1H), 3.73-3.68 (m, | | | | |
| | 2H), 3.50 (s, 3H), 3.03 (t, J = | | | | |
| | 12.0 Hz, 1H), 2.73 (t, J = 12.0 | | | | |
| | Hz, 1H), 2.43 (s, 3H), 1.96 (d, | | | | |
| | J = 5.6 Hz, 3H), 1.02-0.97 (m, | | | | |
| | 2H), 0.94-0.92 (m, 2H). | | | | |
| Ex. 179A | ¹H-NMR (400 MHz, DMSO-d₆): | Condition | CHIRALPAK IC | Yellow | 37.5 |
| (Peak 1) | δ 8.46 (d, J = 5.2 Hz, 1H), 7.86 | K: Rt = | (250 × 4.6)mm, | solid | |
| | (t, J = 8.8 Hz, 2H), 7.75 (d, J = 8.0 | 2.66 min. | 5μ M.P.- | | |
| | Hz, 1H), 7.32 (s, 1H), 7.24 (d, | m/z 529.5 | Hexane/DCM/ | | |
| | J = 4.4 Hz, 1H), 4.74-4.65 (m, | [M + H]⁺. | EtOH/IPAmine: | | |
| | 3H), 3.83 (bs, 1H), 3.51 (s, 3H), | | 50/25/25/0.1 | | |
| | 2.89-2.78 (m, 2H), 2.49 (s, 3H), | | Flow Rate - | | |
| | 2.43 (s, 3H), 1.28 (d, J = 6.0 Hz, | | 1.0 ml/min: | | |
| | 3H). | | Solubility: | | |
| Ex. 179B | ¹H-NMR (400 MHz, DMSO-d₆): | Condition | MeOH, Rt = | Yellow | 35.7 |
| (Peak 2) | δ 8.46 (d, J = 5.2 Hz, 1H), 7.86 | K: Rt = | 5.60 min for | solid | |
| | (t, J = 8.8 Hz, 2H), 7.75 (d, J = 8.0 | 2.66 min. | Peak 1 and Rt = | | |
| | Hz, 1H), 7.32 (s, 1H), 7.24 (d, | m/z 529.5 | 8.14 min for | | |
| | J = 4.4 Hz, 1H), 4.74-4.65 (m, | [M + H]⁺. | Peak 2. | | |
| | 3H), 3.83 (bs, 1H), 3.51 (s, 3H), | | | | |
| | 2.89-2.78 (m, 2H), 2.49 (s, 3H), | | | | |
| | 2.43 (s, 3H), 1.28 (d, J = 6.0 Hz, | | | | |
| | 3H). | | | | |
| Ex. 180A | 1H NMR (400 MHz, DMSO-d6) | Condition | I-CELLULOSE Z | Yellow | 31.2 |
| (Peak 1) | δ 9.40 (s, 1H), 8.69 (d, J = 8.4 | K: Rt = | (150 × 4.6) mm, | solid | |
| | Hz, 1H), 8.43 (d, J = 4.8 Hz, 1H), | 2.54 min. | 3μ M.P.-0.3% | | |
| | 7.82 (d, J = 8.0 Hz, 1H), 7.41 (s, | m/z 507.5 | IPamine in MeOH: | | |
| | 1H), 7.35 (s, 1H), 7.28 (d, J = 4.8 | [M + H]⁺. | Flow Rate - | | |
| | Hz, 1H), 7.04 (t, J = 14.8 Hz, | | 4.0 ml/min | | |
| | 1H), 4.93-4.90 (dd, J = 10.4 Hz, | | Solubility: | | |
| | J = 2.4 Hz, 1H), 4.55 (d, J = 12.0 | | MeOH, Rt = | | |
| | Hz, 1H), 4.29 (d, J = 12.4 Hz, | | 3.53 min for | | |
| | 1H), 3.54 (s, 3H), 2.86 (d, J = | | Peak 1 and Rt = | | |
| | 12.8 Hz, 1H), 2.72-2.66 (m, 1H), | | 4.24 min for | | |
| | 2.54 (s, 3H), 2.50 (s, 3H), 1.36 | | Peak 2. | | |
| | (s, 3H), 1.33 (s, 3H). | | | | |
| Ex. 180B | 1H NMR (400 MHz, DMSO-d6) | Condition | | Yellow | 30.3 |
| (Peak 2) | δ 9.40 (s, 1H), 8.69 (d, J = 8.4 | K: Rt = | | solid | |
| | Hz, 1H), 8.43 (d, J = 4.8 Hz, 1H), | 2.54 min. | | | |
| | 7.82 (d, J = 8.0 Hz, 1H), 7.41 (s, | m/z 507.5 | | | |
| | 1H), 7.35 (s, 1H), 7.28 (d, J = 4.8 | [M + H]⁺. | | | |
| | Hz, 1H), 7.04 (t, J = 14.8 Hz, | | | | |
| | 1H), 4.93-4.90 (dd, J = 10.4 Hz, | | | | |
| | J = 2.4 Hz, 1H), 4.55 (d, J = 12.0 | | | | |
| | Hz, 1H), 4.29 (d, J = 12.4 Hz, | | | | |
| | 1H), 3.54 (s, 3H), 2.86 (d, J = | | | | |
| | 12.8 Hz, 1H), 2.72-2.66 (m, 1H), | | | | |
| | 2.54 (s, 3H), 2.50 (s, 3H), 1.36 | | | | |
| | (s, 3H), 1.33 (s, 3H). | | | | |
| Ex. 181A | 1H NMR (400 MHz, DMSO-d6) | LCMS | I-CELLULOSE Z | Yellow | 37.0 |
| (Peak 1) | δ 9.45 (s, 1H), 8.75-8.72 (dd, | Condition | (150 × 4.6) mm, | solid | |
| | J = 8.0 Hz, J = 1.6 Hz, 1H), 8.51 | K: Rt = | 3μ M.P.-0.3% | | |
| | (d, J = 5.2 Hz, 1H), 7.86 (d, J = 8.0 | 2.57 min. | IPamine in MeOH: | | |
| | Hz, 1H), 7.58 (s, 1H), 7.44 (s, | m/z 515.5 | Flow Rate - | | |
| | 1H), 7.36 (d, J = 4.8 Hz, 1H), | [M + H]⁺. | 4.0 ml/min | | |
| | 7.04 (t, J = 14.8 Hz, 1H), 5.40- | | Solubility: | | |
| | 5.38 (dd, J = 10.8 Hz, J = 2.4 Hz, | | MeOH, Rt = | | |
| | 1H), 4.91 (d, J = 12.8 Hz, 1H), | | 2.39 min for | | |
| | 4.62 (d, J = 13.2 Hz, 1H), 3.70- | | Peak 1 and Rt = | | |
| | 3.61 (m, 1H), 3.55 (s, 3H), 3.26- | | 3.40 min for | | |
| | 3.20 (m, 1H), 2.56 (s, 3H), 2.52 | | Peak 2. | | |
| | (s, 3H). | | | | |
| Ex. 181B | 1H NMR (400 MHz, DMSO-d6) | LCMS | | Yellow | 33.0 |
| (Peak 2) | δ 9.45 (s, 1H), 8.75-8.72 (dd, | Condition | | solid | |
| | J = 8.0 Hz, J = 1.6 Hz, 1H), 8.51 | K: Rt = | | | |
| | (d, J = 5.2 Hz, 1H), 7.86 (d, J = 8.0 | 2.57 min. | | | |
| | Hz, 1H), 7.58 (s, 1H), 7.44 (s, | m/z 515.5 | | | |
| | 1H), 7.36 (d, J = 4.8 Hz, 1H), | [M + H]⁺. | | | |
| | 7.04 (t, J = 14.8 Hz, 1H), 5.40- | | | | |
| | 5.38 (dd, J = 10.8 Hz, J = 2.4 Hz, | | | | |
| | 1H), 4.91 (d, J = 12.8 Hz, 1H), | | | | |
| | 4.62 (d, J = 13.2 Hz, 1H), 3.70- | | | | |
| | 3.61 (m, 1H), 3.55 (s, 3H), 3.26- | | | | |
| | 3.20 (m, 1H), 2.56 (s, 3H), 2.52 | | | | |
| | (s, 3H). | | | | |
| Ex. 182A | 1H NMR (400 MHz, DMSO-d6) | LCMS | Chiralpak IG | Yellow | 31.0 |
| (Peak 1) | δ 8.43 (d, J = 4.8 Hz, 1H), 7.62- | Condition | (4.6 × 250 mm), | solid | |
| | 7.56 (m, 1H), 7.37-7.30 (m, 3H), | K: Rt = | 5μ ARD/K/7788 | | |
| | 7.27 (d, J = 4.8 Hz, 1H), 7.19 (t, | 2.53 min. | Mobile Phase: | | |
| | J = 8.4 Hz, 1H), 4.68 (d, J = 10.4 | m/z 478.4 | Hexane/DCM/ | | |
| | Hz, 1H), 4.40 (d, J = 12 Hz,, 1H), | [M + H]+. | EtOH/IPAmine: | | |
| | 4.31 (d, J = 12.4 Hz, 1H), 3.87 | | 50/25/25/0.1 | | |
| | (brs, 1H), 3.52 (s, 3H), 3.28 (s, | | Flow Rate: | | |
| | 3H), 2.70-2.61 (m, 2H), 2.44 (s, | | 1.0 ml/min | | |
| | 3H), 1.29 (d, J = 6.0 Hz, 3H). | | Solubility: | | |
| Ex. 182B | 1H NMR (400 MHz, DMSO-d6) | LCMS | MeOH; Rt = | Yellow | 32.7 |
| (Peak 2) | δ 8.43 (d, J = 4.8 Hz, 1H), 7.62- | Condition | 5.37 min for | solid | |
| | 7.56 (m, 1H), 7.37-7.30 (m, 3H), | K: Rt = | Peak 1 and Rt = | | |
| | 7.27 (d, J = 4.8 Hz, 1H), 7.19 (t, | 2.53 min. | 7.50 min for | | |
| | J = 8.4 Hz, 1H), 4.68 (d, J = 10.4 | m/z 478.4 | Peak 2. | | |
| | Hz, 1H), 4.40 (d, J = 12 Hz,, 1H), | [M + H]+. | | | |
| | 4.31 (d, J = 12.4 Hz, 1H), 3.87 | | | | |
| | (brs, 1H), 3.52 (s, 3H), 3.28 (s, | | | | |
| | 3H), 2.70-2.61 (m, 2H), 2.44 (s, | | | | |
| | 3H), 1.29 (d, J = 6.0 Hz, 3H). | | | | |
| Ex. 183A | ¹H-NMR (400 MHz, CD₃OD): δ | LCMS | I-CELLULOSE Z | Yellow | 38.2 |
| (Peak 1) | 7.76-7.73 (m, 2H), 7.55 (s, 1H), | Condition | (150 × 4.6) mm, | solid | |
| | 7.13-7.08 (m, 2H), 4.84 (m, 2H), | K: Rt = | 3μ M.P.-0.3% | | |
| | 4.59 (d, J = 10.8 Hz, 1H), 3.80- | 3.16 min. | IPamine in MeOH: | | |
| | 3.79 (m, 1H), 3.71 (s, 3H), 3.62 | m/z 548.5 | Flow Rate - | | |
| | (d, J = 4.0 Hz, 1H), 3.08-3.01 | [M + H]+. | 4.0 ml/min | | |
| | (m, 1H), 2.85-2.79 (m, 1H), | | Solubility: | | |
| | 1.28 (d, J = 6.0 Hz, 3H), 1.07- | | MeOH, Rt = | | |
| | 1.02 (m, 4H). | | 2.07 min for | | |
| Ex. 183B | ¹H-NMR (400 MHz, CD₃OD): δ | LCMS | Peak 1 and Rt = | Yellow | 35.4 |
| (Peak 2) | 7.76-7.73 (m, 2H), 7.55 (s, 1H), | Condition | 2.40 min for | solid | |
| | 7.13-7.08 (m, 2H), 4.84 (m, 2H), | K: Rt = | Peak 2. | | |
| | 4.59 (d, J = 10.8 Hz, 1H), 3.80- | 3.16 min. | | | |
| | 3.79 (m, 1H), 3.71 (s, 3H), 3.62 | m/z 548.5 | | | |
| | (d, J = 4.0 Hz, 1H), 3.08-3.01 | [M + H]+. | | | |
| | (m, 1H), 2.85-2.79 (m, 1H), | | | | |
| | 1.28 (d, J = 6.0 Hz, 3H), 1.07- | | | | |
| | 1.02 (m, 4H). | | | | |
| Ex. 184A | ¹H-NMR (400 MHz, DMSO-d₆): | LCMS | Chiralpak IG | Yellow | 35.3 |
| (Peak 1) | δ 9.49 (s, 1H), 8.77 (d, J = 8.0 | Condition | (4.6 × 250 mm), | solid | |
| | Hz, 1H), 8.02 (d, J = 8.0 Hz, 1H), | P: Rt = | 5μ ARD/K | | |
| | 7.85 (s, 1H), 7.48 (s, 1H), 7.39 | 2.93 min. | Mobile Phase: | | |
| | (s, 1H), 4.58 (d, J = 9.2 Hz, 1H), | m/z 526.1 | Hexane/DCM/ | | |
| | 4.39 (d, J = 12.4 Hz, 1H), 4.32 (d, | [M + H]+. | EtOH/IPAmine: | | |
| | J = 10.8 Hz, 1H), 3.80 (t, J = 6.8 | | 50/25/25/0.1 | | |
| | Hz, 1H), 3.71-3.67 (m, 1H), 3.54 | | Flow Rate: | | |
| | (s, 3H), 2.90 (t J = 12.0 Hz, 1H), | | 1.0 ml/min | | |
| | 2.63 (t, J = 11.6 Hz, 1H), 2.60 (s, | | Solubility: | | |
| | 3H), 1.23 (d, J = 6.0 Hz, 3H), | | MeOH, Rt = | | |
| | 1.03-1.01(m, 2H), 0.98-0.91 | | 5.76 min for | | |
| | (m, 2H). | | Peak 1 and Rt = | | |
| Ex. 184B | ¹H-NMR (400 MHz, DMSO-d₆): | LCMS | 6.25 min for | Yellow | 33.1 |
| (Peak 2) | δ 9.49 (s, 1H), 8.77 (d, J = 8.0 | Condition | | solid | |
| | Hz, 1H), 8.02 (d, J = 8.0 Hz, 1H), | P: Rt = | | | |
| | 7.85 (s, 1H), 7.48 (s, 1H), 7.39 | 2.93 min. | | | |
| | (s, 1H), 4.58 (d, J = 9.2 Hz, 1H), | m/z 526.1 | | | |
| | 4.39 (d, J = 12.4 Hz, 1H), 4.32 (d, | [M + H]+. | | | |
| | J = 10.8 Hz, 1H), 3.80 (t, J = 6.8 | | | | |
| | Hz, 1H), 3.71-3.67 (m, 1H), 3.54 | | | | |
| | (s, 3H), 2.90 (t J = 12.0 Hz, 1H), | | | | |
| | 2.63 (t, J = 11.6 Hz, 1H), 2.60 (s, | | | | |
| | 3H), 1.23 (d, J = 6.0 Hz, 3H), | | | | |
| | 1.03-1.01(m, 2H), 0.98-0.91 | | | | |
| | (m, 2H). | | | | |

### Example 185

### (8-(2,4-difluorophenyl)-2,3-dimethyl-6-((2R,4S,6R)-2-methyl-6-(2-methylpyridin-4-yl)tetrahydro-2H-pyran-4-yl)pyrido[3,4-d]pyrimidin-4 (3H)-one

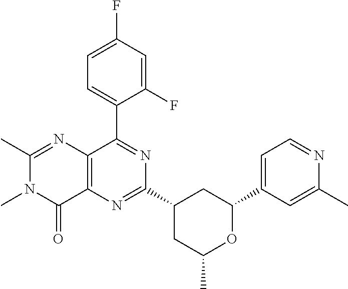

Procedure of Example 168 was followed using 6-chloro-8-(2,4-difluorophenyl)-2,3-dimethylpyrido[3,4-d]pyrimidin-4 (3H)-one and 4-((2R,4S,6R)-4-bromo-6-methyltetrahydro-2H-pyran-2-yl)-2-methylpyridine as starting materials followed by reverse phase prep-HPLC purification to afford (8-(2,4-difluorophenyl)-2,3-dimethyl-6-((2R,4S,6R)-2-methyl-6-(2-methylpyridin-4-yl)tetrahydro-2H-pyran-4-yl)pyrido[3,4-d]pyrimidin-4 (3H)-one (17 mg, 12%, white solid). 4-((2R,4S,6R)-4-bromo-6-methyltetrahydro-2H-pyran-2-yl)-2-methylpyridine was synthesized using FeBr₃ (1.5 eq.) in dichloromethane under room temperature for 16 h in 40% yield according to similar procedure depicted in Step-1, Example 94.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 45% A and 55% B, then 45% A and 55% B in 3 min, then to 25% A and 75% B in 20 min., then to 5% A and 95% B in 21 min., held this composition up to 22 min. for column washing, then returned to initial composition in 23 min. and held till 25 min.

1H NMR (400 MHz, DMSO-d6) δ 8.36 (d, J=4.8 Hz, 1H), 7.88 (s, 1H), 7.59-7.57 (m, 1H), 7.34-7.32 (m, 1H), 7.25-7.16 (m, 3H), 4.58 (d, J=10.8 Hz, 1H), 3.82-3.81 (m, 1H), 3.54 (s, 3H), 3.40-3.37 (m, 1H), 2.49 (s, 3H), 2.44 (s, 3H), 2.20-2.17 (m, 1H), 2.03-2.01 (m, 1H), 1.59-1.52 (m, 2H), 1.26 (d, J=6.0 Hz, 3H).

### Condition K: Rt=2.57 min. m/z 477.4 [M+H]+

### Example 186

### Example 186A: Enantiomer Peak 1

### 6-((2S,4R)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one

### Example 186B: Enantiomer Peak 2

### 6-((2R,4S)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one

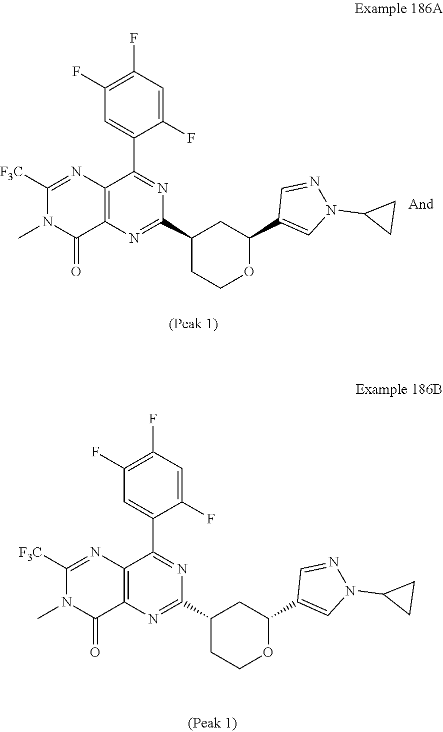 [SMILES: Cn1c(C(F)(F)F)nc2c(-c3cc(F)c(F)cc3F)nc([C@@H]3CCO[C@H](c4cnn(C5CC5)c4)C3)nc2c1=O ; Cn1c(C(F)(F)F)nc2c(-c3cc(F)c(F)cc3F)nc([C@H]3CCO[C@@H](c4cnn(C5CC5)c4)C3)nc2c1=O]

The racemic compound 6-(6-(1-cyclopropyl-1H-pyrazol-4-yl)-3,6-dihydro-2H-pyran-4-yl)-3-methyl-2-(trifluoromethyl)-8-(2,4,5-trifluorophenyl)pyrimido[5,4-d]pyrimidin-4 (3H)-one (30 mg, 25% yield, white solid, purified by prep HPLC method) was synthesized according to similar procedure depicted in Example 17.

Chiral separation of 6-(6-(1-cyclopropyl-1H-pyrazol-4-yl)-3,6-dihydro-2H-pyran-4-yl)-3-methyl-2-(trifluoromethyl)-8-(2,4,5-trifluorophenyl)pyrimido[5,4-d]pyrimidin-4 (3H)-one (30 mg) was done by SFC to afford Example 187A, 6-((2S,4R)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one (7 mg, 23% yield; eluted first, assigned as Peak 1 with arbitrary assignment of stereochemistry) as white solid and Example 187B, 6-((2R,4S)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one (6 mg, 20% yield, eluted later, assigned as Peak 2 with arbitrary assignment of stereochemistry) as white solid.

SFC method: Chiral Prep-purification was done on Pic Solution 175 instrument equipped with Knauer UV Detector 40D by using I-Cellulose-J column (30.0 mm×250 mm) 0.5p operating at 35° C. temperature, maintaining flow rate of 70 mL/min, using 75% CO2 in super critical state & 25% of 100% Methanol as mobile phase. Run this isocratic mixture up to 15.0 minutes and also maintained the isobaric condition of 100 bar at 290 nm wavelength.

Example 186A (Peak 1): 1H NMR (400 MHz, DMSO-d₆) δ 7.91-7.78 (m, 2H), 7.72 (s, 1H), 7.37 (s, 1H), 4.50 (d, J=10.0 Hz, 1H), 4.12-4.08 (dd, J=10.8, J=3.6 Hz, 1H), 3.73-3.63 (m, 2H), 3.61 (s, 3H), 3.53-3.46 (m, 1H), 2.23 (d, J=12.8 Hz, 1H), 2.02-2.1.94 (m, 1H), 1.92-1.83 (m, 2H), 1.01-0.96 (m, 2H), 0.93-0.88 (m, 2H).

LCMS Condition K: Rt=3.08 min. m/z 551.5 [M+H]⁺.

Example 186B (Peak 2): 1H NMR (400 MHz, DMSO-d₆) δ 7.91-7.78 (m, 2H), 7.72 (s, 1H), 7.37 (s, 1H), 4.50 (d, J=10.0 Hz, 1H), 4.12-4.08 (dd, J=10.8, J=3.6 Hz, 1H), 3.73-3.63 (m, 2H), 3.61 (s, 3H), 3.53-3.46 (m, 1H), 2.23 (d, J=12.8 Hz, 1H), 2.02-2.1.94 (m, 1H), 1.92-1.83 (m, 2H), 1.01-0.96 (m, 2H), 0.93-0.88 (m, 2H).

LCMS Condition K: Rt=3.08 min. m/z 551.5 [M+H]⁺.

Analytical chiral HPLC: I-CELLULOSE J (150×4.6) mm, 3μ M.P.—0.3% IPamine in MeOH: Flow Rate—4.0 ml/min Solubility: MeOH, Rt=2.27 min for Peak 1 and Rt=2.64 min for Peak 2.

### Example 187

### 8-(4-chloro-2-fluorophenyl)-3-methyl-6-(2-(1-methyl-1H-pyrazol-4-yl)morpholino)pyrimido[5,4-d]pyrimidin-4 (3H)-one

### Step-1—Preparation of ethyl 5-amino-2,6-dioxo-1,2,3,6-tetrahydropyrimidine-4-carboxylate

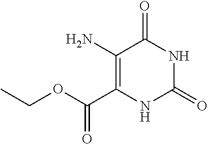

To a stirred solution of ethyl 5-nitro-2,6-dioxo-1,2,3,6-tetrahydropyrimidine-4-carboxylate (800 mg, 3.49 mmol) in water (30 mL) ware added sodium bicarbonate (4 g, 47.51 mmol) and sodium dithionite (3.3 g, 18.86 mmol). The resulting mixture was stirred at room temperature for 2 h. Reaction mixture was filtered and residue was washed with water, dried under vacuum to afford ethyl 5-amino-2,6-dioxo-1,2,3,6-tetrahydropyrimidine-4-carboxylate (390 mg, 56% yield) as white solid.

LCMS Condition C: Rt=0.7 min. m/z 200.2 [M+H]⁺.

### Step-2—Preparation of ethyl (E)-5-((ethoxymethylene)amino)-2,6-dioxo-1,2,3,6-tetrahydropyrimidine-4-carboxylate

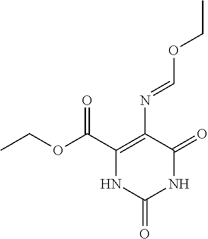 [SMILES: CCO/C=N/c1c(C(=O)OCC)[nH]c(=O)[nH]c1=O]

A stirred solution of ethyl 5-amino-2,6-dioxo-1,2,3,6-tetrahydropyrimidine-4-carboxylate (390 mg, 1.96 mmol) in diethoxymethyl acetate (5 mL) was heated at 185° C. for 2 h. The precipitated solid was filtered, washed with ethyl acetate, dried under vacuum to afford ethyl (E)-5-((ethoxymethylene)amino)-2,6-dioxo-1,2,3,6-tetrahydropyrimidine-4-carboxylate (380 mg, 75% yield) as white solid.

1H NMR (400 MHz, DMSO-d6) δ 11.48 (s, 1H), 10.89 (s, 1H), 8.26 (s, 1H), 4.27-4.22 (m, 2H), 4.17-4.12 (m, 2H), 1.27-1.23 (m, 6H).

### Step-3—Preparation of 7-methyl-1,7-dihydropyrimido[5,4-d]pyrimidine-2,4,8 (3H)-trione

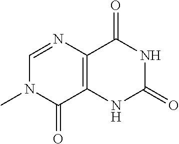

To a stirred solution of ethyl (E)-5-((ethoxymethylene)amino)-2,6-dioxo-1,2,3,6-tetrahydropyrimidine-4-carboxylate (320 mg, 1.25 mmol) in ethanol (10 mL) was added methyl amine solution (2M in THF, 10 mL). The resulting mixture was heated at 100° C. for 16 h. The precipitated solid was filtered, washed with ethanol, dried under vacuum to afford 7-methyl-1,7-dihydropyrimido[5,4-d]pyrimidine-2,4,8 (3H)-trione (200 mg, 82% yield) as white solid.

LCMS Condition N: Rt=0.2 min. m/z 195.3 [M+H]⁺.

### Step-4—Preparation of 6,8-dichloro-3-methylpyrimido[5,4-d]pyrimidin-4 (3H)-one

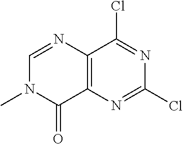

To a stirred solution of 7-methyl-1,7-dihydropyrimido[5,4-d]pyrimidine-2,4,8 (3H)-trione (200 mg, 1.02 mmol) in POCl₃ (4 mL) was added DIPEA (0.8 mL) dropwise under argon atmosphere at room temperature. The resulting mixture was heated at 100° C. for 16 h. The reaction mixture poured into ice cold water, neutralized with sodium bicarbonate, extracted with ethyl acetate, dried over sodium sulphate and concentrated under reduced pressure. The crude product was purified by column chromatography (silica gel; 0-20% ethyl acetate-hexane) to afford 6,8-dichloro-3-methylpyrimido[5,4-d]pyrimidin-4 (3H)-one (60 mg, 25% yield) as white solid.

LCMS Condition N: Rt=1.45 min. m/z 231.1 [M+H]⁺.

### Step-5—Preparation of 6-chloro-8-(4-chloro-2-fluorophenyl)-3-methylpyrimido[5,4-d]pyrimidin-4 (3H)-one

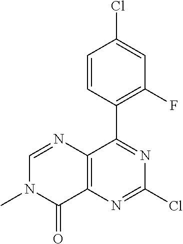 [SMILES: Cn1cnc2c(-c3ccc(Cl)cc3F)nc(Cl)nc2c1=O]

To a stirred solution of 6,8-dichloro-3-methylpyrimido[5,4-d]pyrimidin-4 (3H)-one (50 mg, 0.22 mmol) and (4-chloro-2-fluorophenyl)boronic acid (34 mg 0.19 mmol) in 1,4-dioxane (3 mL) and water (1 mL) was added sodium carbonate (34 mg, 0.32 mmol) and degassed with argon. PdCl₂(dppf) (16 mg, 0.02 mmol) was added to the reaction mixture under inert atmosphere. The resulting mixture was heated at 80° C. for 16 h. Reaction mixture was diluted with ethyl acetate, filtered through a pad of celite and washed with ethyl acetate. Combined organic layer was washed with brine, dried over anhydrous sodium sulphate, filtered and concentrated under reduced pressure. Crude product was purified by column chromatography (silica gel; 35% ethyl acetate-hexane) to afford 6-chloro-8-(4-chloro-2-fluorophenyl)-3-methylpyrimido[5,4-d]pyrimidin-4 (3H)-one (35 mg, 49% yield) as white solid.

LCMS Condition N: Rt=2.07 min. m/z 325.2 [M+H]⁺.

### Step-6—Preparation of 8-(4-chloro-2-fluorophenyl)-3-methyl-6-(2-(1-methyl-1H-pyrazol-4-yl)morpholino)pyrimido[5,4-d]pyrimidin-4 (3H)-one [Example 187]

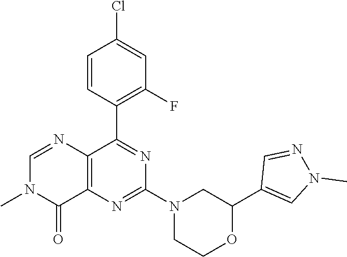

To a stirred solution of 6-chloro-8-(4-chloro-2-fluorophenyl)-3-methylpyrimido[5,4-d]pyrimidin-4 (3H)-one (32 mg, 0.09 mmol) in DMSO (1 mL) were added 2-(1-methyl-1H-pyrazol-4-yl)morpholine (47 mg, 0.12 mmol) and DIPEA (0.07 mL, 0.39 mmol). The resulting mixture was heated at 100° C. for 16 h. Reaction mixture was passed through a pad of celite and washed with dichloromethane and concentrated under reduced pressure. Crude product was purified by reverse phase preparative HPLC to afford 8-(4-chloro-2-fluorophenyl)-3-methyl-6-(2-(1-methyl-1H-pyrazol-4-yl)morpholino)pyrimido[5,4-d]pyrimidin-4 (3H)-one (10 mg, 22% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 70% A and 30% B, then 50% A and 50% B in 3 min, then to 10% A and 90% B in 20 min., then to 5% A and 95% B in 21 min., held this composition up to 22 min. for column washing, then returned to initial composition in 23 min. and held till 25 min.

1H NMR (400 MHz, DMSO-d6) δ 8.14 (s, 1H), 7.47 (s, 1H), 7.66 (t, J=8.0 Hz, 1H), 7.58 (d, J=10.4 Hz, 1H), 7.46-7.45 (m, 2H), 4.62 (d, J=12.0 Hz, 1H), 4.54-4.50 (m, 2H), 3.99 (d, J=12.4 Hz, 1H), 3.81 (s, 3H), 3.66 (t, J=11.6 Hz, 1H), 3.47 (s, 3H), 3.20-3.14 (m, 1H).

LCMS Condition K: Rt=2.67 min. m/z 456.18 [M+H]⁺.

### Example 188

### 8-(4-chloro-2-fluorophenyl)-2,3-dimethyl-6-(2-(trifluoromethyl)-5,6-dihydro-[1,2,4]triazolo[1,5-a]pyrazin-7 (8H)-yl)pyrimido[5,4-d]pyrimidin-4 (3H)-one

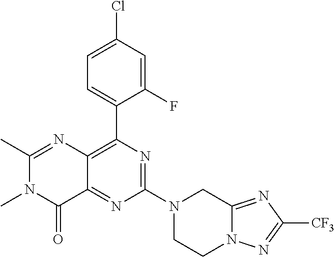 [SMILES: Cc1nc2c(-c3ccc(Cl)cc3F)nc(N3CCn4nc(C(F)(F)F)nc4C3)nc2c(=O)n1C]

To a stirred solution of 6-chloro-8-(4-chloro-2-fluorophenyl)-2,3-dimethylpyrimido[5,4-d]pyrimidin-4 (3H)-one (100 mg, 0.3 mmol) and 2-(trifluoromethyl)-5,6,7,8-tetrahydro-[1,2,4]triazolo[1,5-a]pyrazine (56 mg, 0.44 mmol) in DMSO (2 mL) was added DIPEA (0.1 ml, 0.59 mmol). Resulting solution was heated at 120° C. for 16 h. The crude solution was purified by reverse phase prep-HPLC chromatography to afford 8-(4-chloro-2-fluorophenyl)-2,3-dimethyl-6-(2-(trifluoromethyl)-5,6-dihydro-[1,2,4]triazolo[1,5-a]pyrazin-7 (8H)-yl)pyrimido[5,4-d]pyrimidin-4 (3H)-one (60 mg, 30% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 40% A and 60% B in 3 min, then to 10% A and 90% B in 20 min., then to 5% A and 95% B in 21 min., held this composition up to 22 min. for column washing, then returned to initial composition in 23 min. and held till 25 min.

1H NMR (400 MHz, DMSO-d6) b 7.73 (t, J=7.7 Hz, 1H), 7.65-7.62 (m, 1H), 7.48 (d, J=8.4 Hz, 1H), 5.20 (s, 2H), 4.46-4.40 (m, 4H), 3.53 (s, 3H), 2.46 (s, 3H).

LCMS Condition K: Rt=2.98 min. m/z 493.02 [M−H]⁺.

### Example 189 to 195

Example 189[A-B] to Example 195[A-B]were synthesized using similar procedures as Example 1, Example 103 and Example 112.

Chiral separation details of Example 179A-179B to 185A-185B are captured in following

| Comp-ound No. | | Starting | Chiral separation |
|---|---|---|---|
| Structure | Name | material | specification |
| Ex. 189A (Peak 1) | 8-(2,4-difluorophenyl)- | 8-(2,4- | Similar |
| | 3-methyl-6-((2S,6R)-2- | difluorophenyl)- | specifications as |
| 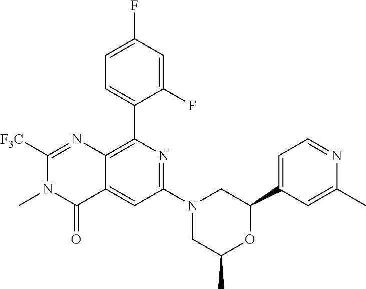 | methyl-6-(2- methylpyridin-4- yl)morpholino)-2- (trifluoromethyl)pyrido [3,4-d]pyrimidin-4(3H)- one | 3-methyl-6-(2- methyl-6-(2- methylpyridin- 4- yl)morpholino)- 2- (trifluoromethyl) pyrido[3,4- d]pyrimidin- 4(3H)-one | example-177 Peak 1 Absolute stereochemistry unknown |
| Ex. 189B (Peak 2) | 8-(2,4-difluorophenyl)- | | Peak 2 |
| | 3-methyl-6-((2R,6S)-2- | | Absolute |
| 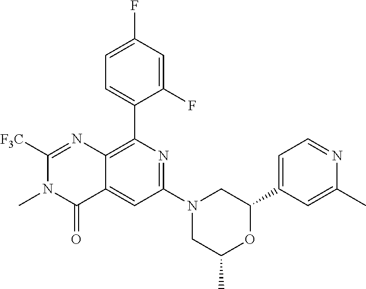 | methyl-6-(2- methylpyridin-4- yl)morpholino)-2- (trifluoromethyl)pyrido [3,4-d]pyrimidin-4(3H)- one | | stereochemistry unknown |
| Ex. 190A (Peak 1) | 6-((2R,6S)-2-(1- | 6-(2-(1- | Similar |
| | cyclopropyl-1H- | cyclopropyl- | specifications as |
| 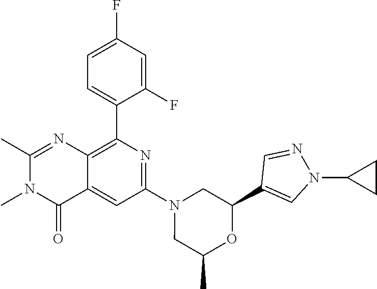 [SMILES: Cc1nc2c(-c3ccc(F)cc3F)nc(N3C[C@@H](c4cnn(C5CC5)c4)O[C@@H](C)C3)cc2c(=O)n1C] | pyrazol-4-yl)-6- methylmorpholino)-8- (2,4-difluorophenyl)- 2,3-dimethylpyrido[3,4- d]pyrimidin-4(3H)-one | 1H-pyrazol-4- yl)-6- methyl- morpholino)- 8-(2,4- difluorophenyl)- 2,3- dimethylpyrido [3,4- d]pyrimidin- 4(3H)-one | example-177 Peak 1 Absolute stereochemistry unknown |
| Ex. 190B (Peak 2) | 6-((2S,6R)-2-(1- | | Peak 2 |
| | cyclopropyl-1H- | | Absolute |
| 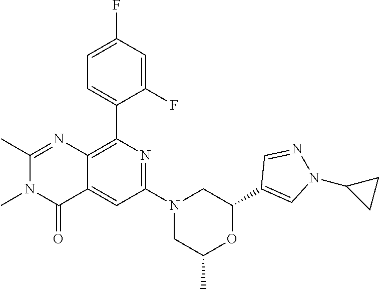 [SMILES: Cc1nc2c(-c3ccc(F)cc3F)nc(N3C[C@@H](C)O[C@@H](c4cnn(C5CC5)c4)C3)cc2c(=O)n1C] | pyrazol-4-yl)-6- methylmorpholino)-8- (2,4-difluorophenyl)- 2,3-dimethylpyrido[3,4- d]pyrimidin-4(3H)-one | | stereochemistry unknown |
| Ex. 191A (Peak 1) | 8-(2,4-difluorophenyl)- | 8-(2,4- | Similar |
| | 3-methyl-6-((2S,6R)-2- | difluorophenyl)- | specifications as |
| 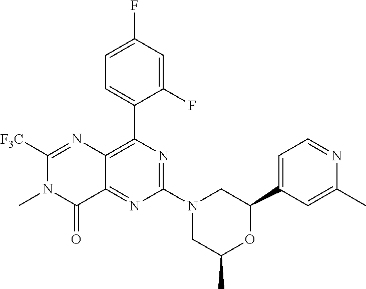 | methyl-6-(2- methylpyridin-4- yl)morpholino)-2- (trifluoromethyl) pyrimido[5,4-d] pyrimidin- 4(3H)-one | 3-methyl-6-(2- methyl-6-(2- methylpyridin- 4- yl)morpholino)- 2- (trifluoromethyl) pyrimido[5,4- d]pyrimidin- 4(3H)-one | example-177 Peak 1 Absolute stereochemistry unknown |
| Ex. 191B (Peak 2) | 8-(2,4-difluorophenyl)- | | Peak 2 |
| | 3-methyl-6-((2R,6S)-2- | | Absolute |
| 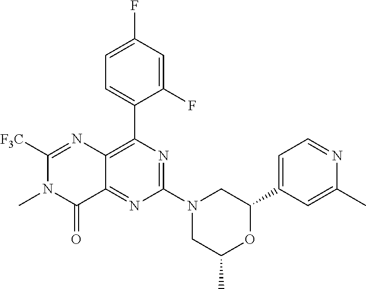 | methyl-6-(2- methylpyridin-4- yl)morpholino)-2- (trifluoromethyl) pyrimido[5,4-d] pyrimidin- 4(3H)-one | | stereochemistry unknown |
| Ex. 192A (Peak 1) | 8-(6- | 8-(6- | Similar |
| | (difluoromethyl)pyridin- | (difluoromethyl) | specifications as |
| 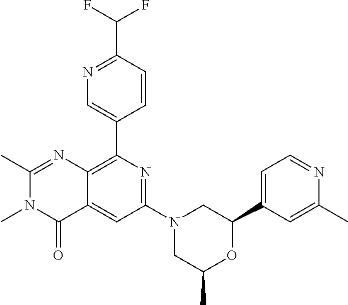 | 3-yl)-2,3-dimethyl-6- ((2S,6R)-2-methyl-6- (2-methylpyridin-4- yl)morpholino)pyrido[3, 4-d]pyrimidin-4(3H)- one | pyridin-3-yl)- 2,3-dimethyl-6- (2-methyl-6-(2- methylpyridin- 4- yl)morpholino) pyrido[3,4- d]pyrimidin- 4(3H)-one | example-177 Peak 1 Absolute stereochemistry unknown |
| Ex. 192B (Peak 2) | 8-(6- | | Peak 2 |
| | (difluoromethyl)pyridin- | | Absolute |
| 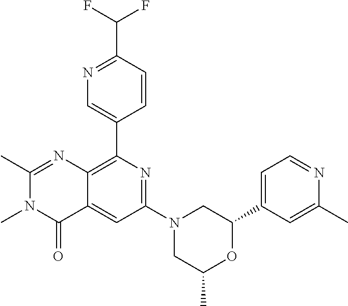 | 3-yl)-2,3-dimethyl-6- ((2R,6S)-2-methyl-6- (2-methylpyridin-4- yl)morpholino)pyrido[3, 4-d]pyrimidin-4(3H)- one | | stereochemistry unknown |
| Ex. 193A (Peak 1) | 6-((2R,6S)-2-(1- | 6-(2-(1- | Similar |
| | cyclopropyl-1H- | cyclopropyl- | specifications as |
| 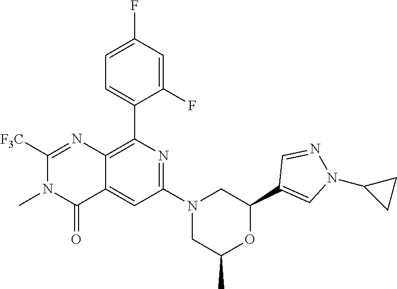 [SMILES: C[C@H]1CN(c2cc3c(=O)n(C)c(C(F)(F)F)nc3c(-c3ccc(F)cc3F)n2)C[C@@H](c2cnn(C3CC3)c2)O1] | pyrazol-4-yl)-6- methylmorpholino)-8- (2,4-difluorophenyl)-3- methyl-2- (trifluoromethyl)pyrido [3,4-d]pyrimidin-4(3H)- one | 1H-pyrazol-4- yl)-6- methyl- morpholino)- 8-(2,4- difluorophenyl)- 3-methyl-2- (trifluoromethyl) pyrido[3,4- d]pyrimidin- 4(3H)-one | example-177 Peak 1 Absolute stereochemistry unknown |
| Ex. 193B (Peak 2) | 6-((2S,6R)-2-(1- | | Peak 2 |
| | cyclopropyl-1H- | | Absolute |
| 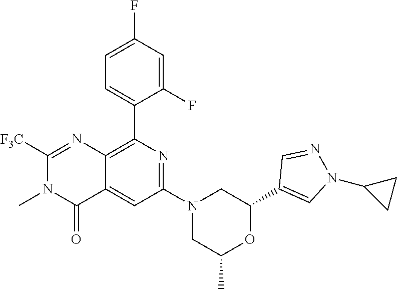 [SMILES: C[C@@H]1CN(c2cc3c(=O)n(C)c(C(F)(F)F)nc3c(-c3ccc(F)cc3F)n2)C[C@H](c2cnn(C3CC3)c2)O1] | pyrazol-4-yl)-6- methylmorpholino)-8- (2,4-difluorophenyl)-3- methyl-2- (trifluoromethyl)pyrido [3,4-d]pyrimidin-4(3H)- one | | stereochemistry unknown |
| Ex. 194A (Peak 1) | 6-((2R,6S)-2-(1- | 6-(2-(1- | Similar |
| | cyclopropyl-1H- | cyclopropyl- | specifications as |
| 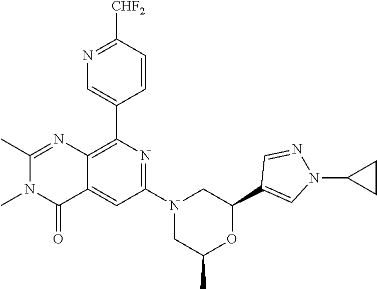 [SMILES: Cc1nc2c(-c3ccc(C(F)F)nc3)nc(N3C[C@@H](c4cnn(C5CC5)c4)O[C@@H](C)C3)cc2c(=O)n1C] | pyrazol-4-yl)-6- methylmorpholino)- 8-(6- (difluoromethyl) pyridin-3-yl)-2,3- dimethylpyrido[3,4- d]pyrimidin-4(3H)-one | 1H-pyrazol-4- yl)-6- methyl- morpholino)- 8-(6- (difluoromethyl) pyridin-3-yl)- 2,3- dimethylpyrido [3,4- d]pyrimidin- 4(3H)-one | example-177 Peak 1 Absolute stereochemistry unknown |
| Ex. 194B (Peak 2) | 6-((2S,6R)-2-(1- | | Peak 2 |
| | cyclopropyl-1H- | | Absolute |
| 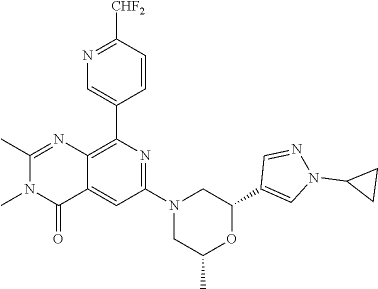 [SMILES: Cc1nc2c(-c3ccc(C(F)F)nc3)nc(N3C[C@@H](C)O[C@@H](c4cnn(C5CC5)c4)C3)cc2c(=O)n1C] | pyrazol-4-yl)-6- methylmorpholino)- 8-(6- (difluoromethyl) pyridin-3-yl)-2,3- dimethylpyrido[3,4- d]pyrimidin-4(3H)-one | | stereochemistry unknown |
| Ex. 195A (Peak 1) | 6-((2R,6S)-2-(1- | 6-(2-(1- | Similar |
| | cyclopropyl-1H- | cyclopropyl- | specifications as |
| 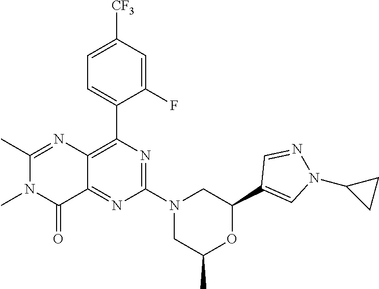 [SMILES: Cc1nc2c(-c3ccc(C(F)(F)F)cc3F)nc(N3C[C@@H](c4cnn(C5CC5)c4)O[C@@H](C)C3)nc2c(=O)n1C] | pyrazol-4-yl)-6- methylmorpholino)-8- (2-fluoro-4- (trifluoromethyl) phenyl)-2,3- dimethylpyrimido [5,4- d]pyrimidin-4(3H)-one | 1H-pyrazol-4- yl)-6- methyl- morpholino)- 8-(2-fluoro-4- (trifluoromethyl) phenyl)-2,3- dimethyl- pyrimido[5,4- d]pyrimidin- 4(3H)-one | example-177 Peak 1 Absolute stereochemistry unknown |
| Ex. 195B (Peak 2) | 6-((2S,6R)-2-(1- | | Peak 2 |
| | cyclopropyl-1H- | | Absolute |
| 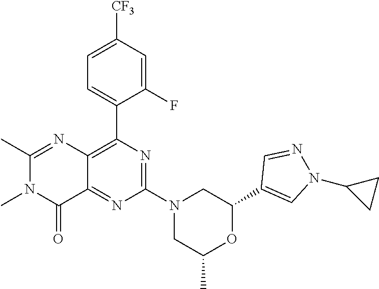 [SMILES: Cc1nc2c(-c3ccc(C(F)(F)F)cc3F)nc(N3C[C@@H](C)O[C@@H](c4cnn(C5CC5)c4)C3)nc2c(=O)n1C] | pyrazol-4-yl)-6- methylmorpholino)-8- (2-fluoro-4- (trifluoromethyl) phenyl)-2,3- dimethylpyrimido [5,4- d]pyrimidin-4(3H)-one | | stereochemistry unknown |

Following table describes analytical data analysis and yield information of examples 189A-189B to examples 195A-195B:

| Compound | | | Analytical | | Yield |
|---|---|---|---|---|---|
| No. | HNMR | LCMS | chiral HPLC | Description | (%) |
| Ex. 189A | ¹H-NMR (400 MHz, DMSO-d₆): δ | Condition | Chiralpak IG | White | 37.8 |
| (Peak 1) | 8.46 (d, J = 5.2 Hz, 1H), 7.67-7.65 | P: Rt = | (4.6 × 250 mm), | solid | |
| | (m, 1H), 7.47 (s, 1H), 7.41-7.32 (m, | 3.36 min. | 5μ ARD/K/ | | |
| | 3H), 7.24-7.23 (m, 1H), 4.72-4.69 | m/z 532.3 | Mobile Phase: | | |
| | (dd, J = 10.4 Hz, J = 2.4 Hz, 1H), | [M + H]⁺. | Hexane/DCM/ | | |
| | 4.53 (d, J = 12.4 Hz, 1H), 4.43 (d, | | EtOH/IPAmine: | | |
| | J = 12.4 Hz, 1H), 3.90 (brs, 1H), | | 50/25/25/0.1 | | |
| | 3.57 (s, 3H), 2.81-2.70 (m, 2H), | | Flow Rate: | | |
| | 2.49 (s, 3H), 1.29 (d, J = 6.0 Hz, | | 1.0 ml/min | | |
| | 3H). | | Solubility: | | |
| Ex. 189B | ¹H-NMR (400 MHz, DMSO-d₆): δ | Condition | MeOH, Rt = | White | 39.6 |
| (Peak 2) | 8.46 (d, J = 5.2 Hz, 1H), 7.67-7.65 | P: Rt = | 4.36 min for | solid | |
| | (m, 1H), 7.47 (s, 1H), 7.41-7.32 (m, | 3.36 min. | Peak 1 and Rt = | | |
| | 3H), 7.24-7.23 (m, 1H), 4.72-4.69 | m/z 532.3 | 5.51 min for | | |
| | (dd, J = 10.4 Hz, J = 2.4 Hz, 1H), | [M + H]⁺. | Peak 2. | | |
| | 4.53 (d, J = 12.4 Hz, 1H), 4.43 (d, | | | | |
| | J = 12.4 Hz, 1H), 3.90 (brs, 1H), | | | | |
| | 3.57 (s, 3H), 2.81-2.70 (m, 2H), | | | | |
| | 2.49 (s, 3H), 1.29 (d, J = 6.0 Hz, | | | | |
| | 3H). | | | | |
| Ex. 190A | ¹H-NMR (400 MHz, DMSO-d₆): δ | Condition | C-AMYLOSE A | Yellow | 37.5 |
| (Peak 1) | 7.83 (s, 1H), 7.58-7.57 (m, 1H), | K: Rt = | (150 × 4.6) mm, | solid | |
| | 7.46 (s, 1H), 7.34-7.31 (m, 2H), | 2.99 min. | 3μ M.P.-0.3% | | |
| | 7.18 (t, J = 8.0 Hz, 1H), 4.55 (d, | m/z 493.5 | IPamine in MeOH: | | |
| | J = 10.4 Hz, 1H), 4.31-4.22 (m, 2H), | [M + H]⁺. | Flow Rate - | | |
| | 3.77 (brs, 1H), 3.66 (d, J = 4.0 Hz, | | 4.0 ml/min | | |
| | 1H), 3.51 (s, 3H), 2.84 (t, J = 12.0 | | Solubility: | | |
| | Hz, 1H), 2.60-2.57 (m, 1H), 2.43 | | MeOH, Rt = | | |
| | (s, 3H), 1.20 (d, J = 6.0 Hz, 3H), | | 4.18 min for | | |
| | 1.01 (brs, 2H), 0.93-0.92 (m, 2H). | | Peak 1 and Rt = | | |
| Ex. 190B | ¹H-NMR (400 MHz, DMSO-d₆): δ | Condition | 5.35 min for | Yellow | 35.7 |
| (Peak 2) | 7.83 (s, 1H), 7.58-7.57 (m, 1H), | K: Rt = | Peak 2. | solid | |
| | 7.46 (s, 1H), 7.34-7.31 (m, 2H), | 2.99 min. | | | |
| | 7.18 (t, J = 8.0 Hz, 1H), 4.55 (d, | m/z 493.5 | | | |
| | J = 10.4 Hz, 1H), 4.31-4.22 (m, 2H), | [M + H]⁺. | | | |
| | 3.77 (brs, 1H), 3.66 (d, J = 4.0 Hz, | | | | |
| | 1H), 3.51 (s, 3H), 2.84 (t, J = 12.0 | | | | |
| | Hz, 1H), 2.60-2.57 (m, 1H), 2.43 | | | | |
| | (s, 3H), 1.20 (d, J = 6.0 Hz, 3H), | | | | |
| | 1.01 (brs, 2H), 0.93-0.92 (m, 2H). | | | | |
| Ex. 191A | ¹H-NMR (400 MHz, DMSO-d₆): δ | Condition | CHIRALPAK IG | Yellow | 33.6 |
| (Peak 1) | 8.45 (d, J = 2.8 Hz, 1H), 7.80-7.74 | K: Rt = | (150 × 4.6) mm, | solid | |
| | (m, 1H), 7.49-7.44 (m, 1H), 7.31- | 2.75 min. | 3μ M.P.-0.3% | | |
| | 7.23 (m, 3H), 4.78 (brs, 2H), 4.65 | m/z 533.4 | IPamine in MeOH: | | |
| | (d, J = 10.4 Hz, 1H), 3.84 (brs, 1H), | [M + H]⁺. | Flow Rate - | | |
| | 3.57 (s, 3H), 2.98-2.85 (m, 2H), | | 4.0 ml/min | | |
| | 2.49 (s, 3H), 1.23 (d, J = 6.0 Hz, | | Solubility: | | |
| | 3H). | | MeOH, Rt = | | |
| Ex. 191B | ¹H-NMR (400 MHz, DMSO-d₆): δ | Condition | 2.04 min for | Yellow | 45.8 |
| (Peak 2) | 8.45 (d, J = 2.8 Hz, 1H), 7.80-7.74 | K: Rt = | Peak 1 and Rt = | solid | |
| | (m, 1H), 7.49-7.44 (m, 1H), 7.31- | 2.75 min. | 2.70 min for | | |
| | 7.23 (m, 3H), 4.78 (brs, 2H), 4.65 | m/z 533.4 | Peak 2. | | |
| | (d, J = 10.4 Hz, 1H), 3.84 (brs, 1H), | [M + H]⁺. | | | |
| | 3.57 (s, 3H), 2.98-2.85 (m, 2H), | | | | |
| | 2.49 (s, 3H), 1.23 (d, J = 6.0 Hz, | | | | |
| | 3H). | | | | |
| Ex. 192A | 1H NMR (400 MHz, DMSO-d6) δ | LCMS | CHIRALCEL OX-H | Yellow | 45.1 |
| (Peak 1) | 9.39 (s, 1H), 8.68 (d, J = 8.0 Hz, | Condition | (150 × 4.6) mm, | solid | |
| | 1H), 8.44 (d, J = 5.2 Hz, 1H), 7.82 | K: Rt = | 3μ M.P.- | | |
| | (d, J = 8.0 Hz, 1H), 7.42 (s, 1H), | 2.40 min. | ACN/MEOH/ | | |
| | 7.36 (s, 1H), 7.29 (d, J = 4.8 Hz, | m/z 493.5 | IPAMINE | | |
| | 1H), 7.03 (t, J = 14.8 Hz, 2H), 4.68 | [M + H]⁺. | (50/50/0.3) | | |
| | (d, J = 10.4 Hz, 1H), 4.49 (d, J = 12.0 | | Flow Rate - | | |
| | Hz, 1H), 4.38 (d, J = 12.4 Hz, 1H), | | 4.0 ml/min | | |
| | 3.90-3.87 (m, 1H), 3.54 (s, 3H), | | Solubility: | | |
| | 2.74-2.65 (m, 2H), 2.54 (s, 3H), | | MeOH, Rt = | | |
| | 2.49 (s, 3H), 1.31 (d, J = 6.4 Hz, | | 3.46 min for | | |
| | 3H). | | Peak 1 and Rt = | | |
| Ex. 192B | 1H NMR (400 MHz, DMSO-d6) δ | LCMS | 4.01 min for | Yellow | 44.2 |
| (Peak 2) | 9.39 (s, 1H), 8.68 (d, J = 8.0 Hz, | Condition | Peak 2. | solid | |
| | 1H), 8.44 (d, J = 5.2 Hz, 1H), 7.82 | K: Rt = | | | |
| | (d, J = 8.0 Hz, 1H), 7.42 (s, 1H), | 2.40 min. | | | |
| | 7.36 (s, 1H), 7.29 (d, J = 4.8 Hz, | m/z 493.5 | | | |
| | 1H), 7.03 (t, J = 14.8 Hz, 2H), 4.68 | [M + H]⁺. | | | |
| | (d, J = 10.4 Hz, 1H), 4.49 (d, J = 12.0 | | | | |
| | Hz, 1H), 4.38 (d, J = 12.4 Hz, 1H), | | | | |
| | 3.90-3.87 (m, 1H), 3.54 (s, 3H), | | | | |
| | 2.74-2.65 (m, 2H), 2.54 (s, 3H), | | | | |
| | 2.49 (s, 3H), 1.31 (d, J = 6.4 Hz, | | | | |
| | 3H). | | | | |
| Ex. 193A | ¹H-NMR (400 MHz, DMSO-d₆): δ | LCMS | Chiralpak IG | Yellow | 23.0 |
| (Peak 1) | 7.84 (s, 1H), 7.67-7.61 (m, 1H), | Condition | (4.6 × 250 mm), | solid | |
| | 7.47 (s, 1H), 7.41 (s, 1H), 7.37- | K: Rt = | 5μ ARD/K/ | | |
| | 7.34 (m, 1H), 7.22 (t, J = 8.4 Hz, | 3.37 min. | Mobile Phase: | | |
| | 1H), 4.55 (d, J = 8.8 Hz, 1H), 4.46- | m/z 547.5 | Hexane/DCM/ | | |
| | 4.36 (m, 2H), 3.79-3.75 (m, 1H), | [M + H]+. | EtOH/IPAmine: | | |
| | 3.71-3.65 (m, 1H), 3.56 (s, 3H), | | 50/25/25/0.1 | | |
| | 2.94 (t, J = 11.6 Hz, 1H), 2.70-2.65 | | Flow Rate: | | |
| | (m, 1H), 1.21 (d, J = 6.4 Hz, 3H), | | 1.0 ml/min | | |
| | 1.03-0.95 (m, 2H), 0.93-0.85 (m, | | Solubility: | | |
| | 2H). | | MeOHRt = | | |
| Ex. 193B | ¹H-NMR (400 MHz, DMSO-d₆): δ | LCMS | 6.42 min for | Yellow | 24.7 |
| (Peak 2) | 7.84 (s, 1H), 7.67-7.61 (m, 1H), | Condition | Peak 1 and Rt = | solid | |
| | 7.47 (s, 1H), 7.41 (s, 1H), 7.37- | K: Rt = | 7.95 min for | | |
| | 7.34 (m, 1H), 7.22 (t, J = 8.4 Hz, | 3.37 min. | Peak 2. | | |
| | 1H), 4.55 (d, J = 8.8 Hz, 1H), 4.46- | m/z 547.5 | | | |
| | 4.36 (m, 2H), 3.79-3.75 (m, 1H), | [M + H]+. | | | |
| | 3.71-3.65 (m, 1H), 3.56 (s, 3H), | | | | |
| | 2.94 (t, J = 11.6 Hz, 1H), 2.70-2.65 | | | | |
| | (m, 1H), 1.21 (d, J = 6.4 Hz, 3H), | | | | |
| | 1.03-0.95 (m, 2H), 0.93-0.85 (m, | | | | |
| | 2H). | | | | |
| Ex. 194A | ¹H-NMR (400 MHz, DMSO-d₆): δ | LCMS | CHIRALPAK IG | Yellow | 34.2 |
| (Peak 1) | 9.39 (s, 1H), 8.69-8.66 (dd, J = 8.0 | Condition | (150 × 4.6) mm, | solid | |
| | Hz, J = 1.2 Hz, 1H), 7.85-7.81 (m, | K: Rt = | 3μ M.P.- | | |
| | 2H), 7.48 (s, 1H), 7.36 (s, 1H), 7.03 | 2.96 min. | ACN/MEOH/ | | |
| | (t, J = 15.6 Hz, 1H), 4.60-4.57 (dd, | m/z 508.4 | IPAMINE | | |
| | J = 10.4 Hz, J = 2.0 Hz, 1H), 4.42- | [M + H]+. | (50/50/0.3) | | |
| | 4.32 (m, 2H), 3.82-3.77 (m, 1H), | | Flow Rate - | | |
| | 3.72-3.67 (m, 1H), 3.54 (s, 3H), | | 4.0 ml/min | | |
| | 2.89 (t, J = 12.0 Hz, 1H), 2.66-2.59 | | Solubility: | | |
| | (m, 1H), 2.54 (s, 3H), 1.23 (d, | | MeOH, Rt = | | |
| | J = 6.0 Hz, 3H), 1.05-1.01 (m, 2H), | | 3.91 min for | | |
| | 0.98-0.96 (m, 2H). | | Peak 1 and Rt = | | |
| Ex. 194B | ¹H-NMR (400 MHz, DMSO-d₆): δ | LCMS | 4.39 min for | Yellow | 33.4 |
| (Peak 2) | 9.39 (s, 1H), 8.69-8.66 (dd, J = 8.0 | Condition | Peak 2. | solid | |
| | Hz, J = 1.2 Hz, 1H), 7.85-7.81 (m, | K: Rt = | | | |
| | 2H), 7.48 (s, 1H), 7.36 (s, 1H), 7.03 | 2.96 min. | | | |
| | (t, J = 15.6 Hz, 1H), 4.60-4.57 (dd, | m/z 508.4 | | | |
| | J = 10.4 Hz, J = 2.0 Hz, 1H), 4.42- | [M + H]+. | | | |
| | 4.32 (m, 2H), 3.82-3.77 (m, 1H), | | | | |
| | 3.72-3.67 (m, 1H), 3.54 (s, 3H), | | | | |
| | 2.89 (t, J = 12.0 Hz, 1H), 2.66-2.59 | | | | |
| | (m, 1H), 2.54 (s, 3H), 1.23 (d, | | | | |
| | J = 6.0 Hz, 3H), 1.05-1.01 (m, 2H), | | | | |
| | 0.98-0.96 (m, 2H). | | | | |
| Ex. 195A | ¹H-NMR (400 MHz, DMSO-d₆): δ | LCMS | (R,R) | Yellow | 29.2 |
| (Peak 1) | 7.87-7.85 (m, 3H), 7.76 (d, J = 7.6 | Condition | WHELK-O1 | solid | |
| | Hz, 1H), 7.46 (s, 1H), 4.64 (t, | K: Rt = | (4.6 × 250 mm), | | |
| | J = 10.8 Hz, 2H), 4.51(d, J = 10.8 | 3.11 min. | 5μ Mobile | | |
| | Hz, 1H), 3.73-3.67 (m, 2H), 3.51 | m/z 544.5 | Phase: 0.3% | | |
| | (s, 3H), 3.04 (t, J = 12.0 Hz, 1H), | [M + H]+. | IPamine MeOH | | |
| | 2.77-2.66 (m, 1H), 2.43 (s, 3H), | | Flow Rate: | | |
| | 1.19 (d, J = 5.6 Hz, 3H), 1.02-0.97 | | 1.0 ml/min | | |
| | (m, 2H), 0.95-0.90 (m, 2H). | | Solubility: | | |
| Ex. 195B | ¹H-NMR (400 MHz, DMSO-d₆): δ | LCMS | MeOH, Rt = | Yellow | 28.4 |
| (Peak 2) | 7.87-7.85 (m, 3H), 7.76 (d, J = 7.6 | Condition | 3.17 min for | solid | |
| | Hz, 1H), 7.46 (s, 1H), 4.64 (t, | K: Rt = | Peak 1 and Rt = | | |
| | J = 10.8 Hz, 2H), 4.51(d, J = 10.8 | 3.11 min. | 4.03 min for | | |
| | Hz, 1H), 3.73-3.67 (m, 2H), 3.51 | m/z 544.5 | | | |
| | (s, 3H), 3.04 (t, J = 12.0 Hz, 1H), | [M + H]+. | | | |
| | 2.77-2.66 (m, 1H), 2.43 (s, 3H), | | | | |
| | 1.19 (d, J = 5.6 Hz, 3H), 1.02-0.97 | | | | |
| | (m, 2H), 0.95-0.90 (m, 2H). | | | | |

### Example 196 and 197

Example 196[A-B] to Example 197[A-B]were synthesized using a similar procedure to Example 118.

Chiral separation details of Example 196A-196B to 197A-197B are captured in following table:

| | | | Chiral |
|---|---|---|---|
| Comp-ound No. | | Starting | separation |
| Structure | Name | material | specification |
| Ex. 196A (Peak 1) | 6-((2S,4R)-2-(1- | 6-(2-(1- | Similar |
| | cyclopropyl-1H- | cyclopropyl-1H- | specifications as |
| 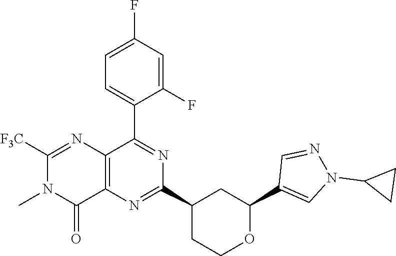 [SMILES: Cn1c(C(F)(F)F)nc2c(-c3ccc(F)cc3F)nc([C@@H]3CCO[C@H](c4cnn(C5CC5)c4)C3)nc2c1=O] | pyrazol-4- yl)tetrahydro-2H- pyran-4-yl)-8-(2,4- difluorophenyl)-3- methyl-2- (trifluoromethyl) pyrimido[5,4-d] pyrimidin- 4(3H)-one | pyrazol-4- yl)tetrahydro- 2H-pyran-4-yl)- 8-(2,4- difluorophenyl)- 3-methyl-2- (trifluoromethyl) pyrimido[5,4- d]pyrimidin- 4(3H)-one | example-177 Peak 1 Absolute stereochemistry unknown |
| Ex. 196B (Peak 2) | 6-((2R,4S)-2-(1- | | Peak 2 |
| | cyclopropyl-1H- | | Absolute |
| 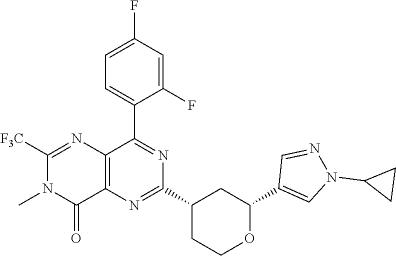 [SMILES: Cn1c(C(F)(F)F)nc2c(-c3ccc(F)cc3F)nc([C@H]3CCO[C@@H](c4cnn(C5CC5)c4)C3)nc2c1=O] | pyrazol-4- yl)tetrahydro-2H- pyran-4-yl)-8-(2,4- difluorophenyl)-3- methyl-2- (trifluoromethyl) pyrimido[5,4-d] pyrimidin- 4(3H)-one | | stereochemistry unknown |
| Ex. 197A (Peak 1) | 6-((2S,4R)-2-(1- | 6-(2-(1- | Similar |
| | cyclopropyl-1H- | cyclopropyl-1H- | specifications as |
| 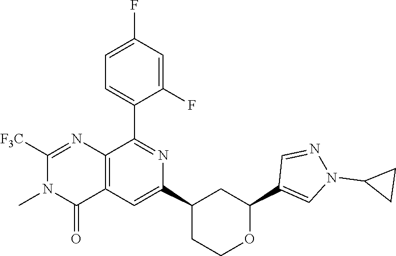 [SMILES: Cn1c(C(F)(F)F)nc2c(-c3ccc(F)cc3F)nc([C@@H]3CCO[C@H](c4cnn(C5CC5)c4)C3)cc2c1=O] | pyrazol-4- yl)tetrahydro-2H- pyran-4-yl)-8-(2,4- difluorophenyl)-3- methyl-2- (trifluoromethyl)pyrido [3,4-d]pyrimidin-4(3H)- one | pyrazol-4- yl)tetrahydro- 2H-pyran-4-yl)- 8-(2,4- difluorophenyl)- 3-methyl-2- (trifluoromethyl) pyrido[3,4- d]pyrimidin- 4(3H)-one | example-177 Peak 1 Absolute stereochemistry unknown |
| Ex. 197B (Peak 2) | 6-((2R,4S)-2-(1- | | Peak 2 |
| | cyclopropyl-1H- | | Absolute |
| 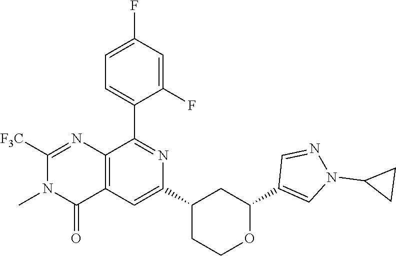 [SMILES: Cn1c(C(F)(F)F)nc2c(-c3ccc(F)cc3F)nc([C@H]3CCO[C@@H](c4cnn(C5CC5)c4)C3)cc2c1=O] | pyrazol-4- yl)tetrahydro-2H- pyran-4-yl)-8-(2,4- difluorophenyl)-3- methyl-2- (trifluoromethyl)pyrido [3,4-d]pyrimidin-4(3H)- onedimethylpyrido [3,4- d]pyrimidin-4(3H)-one | | stereochemistry unknown |

Following table describes analytical data analysis and yield information of examples 196A-196B to examples 197A-197B:

| Compound | | | Analytical | | Yield |
|---|---|---|---|---|---|
| No. | HNMR | LCMS | chiral HPLC | Description | (%) |
| Ex. 196A | 1H NMR (400 MHz, DMSO-d₆) δ | Condition | I CELLULOSE J | White | 20.8 |
| (Peak 1) | 7.79 (d, J = 6.8 Hz, 1H), 7.71 (s, | K: Rt = | (150 × 4.6) mm, | solid | |
| | 1H), 7.52-7.46 (m, 1H), 7.37-7.31 | 3.04 min. | 3μ M.P 0.3% | | |
| | (m, 2H), 4.50 (d, J = 10.0 Hz, 1H), | m/z 533.5 | IPamine in MeOH | | |
| | 4.12-4.08 (dd, J = 11.2 Hz, J = 3.2 | [M + H]⁺. | Flow Rate: | | |
| | Hz, 1H), 3.73-3.67 (m, 2H), 3.60 | | 1.0 ml/min; | | |
| | (s, 3H), 3.50-3.48 (m, 1H), 2.23 | | Solubility: | | |
| | (d, J = 12.8 Hz, 1H), 1.99-1.86 (m, | | MeOH, Rt = | | |
| | 3H), 1.01-0.97 (m, 2H), 0.92-0.89 | | 1.63 min for | | |
| | (m, 2H). | | Peak 1 and Rt = | | |
| Ex. 196B | 1H NMR (400 MHz, DMSO-d₆) δ | Condition | 1.85 min for | White | 20.1 |
| (Peak 2) | 7.79 (d, J = 6.8 Hz, 1H), 7.71 (s, | K: Rt = | Peak 2. | solid | |
| | 1H), 7.52-7.46 (m, 1H), 7.37-7.31 | 3.04 min. | | | |
| | (m, 2H), 4.50 (d, J = 10.0 Hz, 1H), | m/z 533.5 | | | |
| | 4.12-4.08 (dd, J = 11.2 Hz, J = 3.2 | [M + H]⁺. | | | |
| | Hz, 1H), 3.73-3.67 (m, 2H), 3.60 | | | | |
| | (s, 3H), 3.50-3.48 (m, 1H), 2.23 | | | | |
| | (d, J = 12.8 Hz, 1H), 1.99-1.86 (m, | | | | |
| | 3H), 1.01-0.97 (m, 2H), 0.92-0.89 | | | | |
| | (m, 2H). | | | | |
| Ex. 197A | 1H NMR (400 MHz, DMSO-d₆) δ | Condition | CHIRALPAK IG | White | 21.4 |
| (Peak 1) | 8.02 (s, 1H), 7.72 (s, 1H), 7.68- | K: Rt = | (250 × 4.6)mm, | solid | |
| | 7.67 (m, 1H), 7.41-7.37 (m, 2H), | 3.18 min. | 5μ, M.P.- | | |
| | 7.27-7.26 (m, 1H), 4.46 (d, | m/z 532.5 | Hexane/DCM/ | | |
| | J = 10.8 Hz, 1H), 4.06 (d, J = 10.8 | [M + H]⁺. | EtOH/IPAmine: | | |
| | Hz, 1H), 3.68-3.63 (m, 2H), 3.59 | | 50/25/25/0.1 | | |
| | (s, 3H), 3.38-3.31 (m, 1H), 2.14 | | Flow Rate - | | |
| | (d, J = 13.2 Hz, 1H), 1.89-1.83 (m, | | 1.0 ml/min, | | |
| | 3H), 0.98-0.96 (m, 2H), 0.92-0.89 | | Solubility: | | |
| | (m, 2H). | | MeOH, Rt = | | |
| Ex. 197B | 1H NMR (400 MHz, DMSO-d₆) δ | Condition | 6.18 min for | White | 22.2 |
| (Peak 2) | 8.02 (s, 1H), 7.72 (s, 1H), 7.68- | K: Rt = | Peak 1 and Rt = | solid | |
| | 7.67 (m, 1H), 7.41-7.37 (m, 2H), | 3.18 min. | 9.69 min for | | |
| | 7.27-7.26 (m, 1H), 4.46 (d, | m/z 532.5 | Peak 2. | | |
| | J = 10.8 Hz, 1H), 4.06 (d, J = 10.8 | [M + H]⁺. | | | |
| | Hz, 1H), 3.68-3.63 (m, 2H), 3.59 | | | | |
| | (s, 3H), 3.38-3.31 (m, 1H), 2.14 | | | | |
| | (d, J = 13.2 Hz, 1H), 1.89-1.83 (m, | | | | |
| | 3H), 0.98-0.96 (m, 2H), 0.92-0.89 | | | | |
| | (m, 2H). | | | | |

### Example 198

### 5-chloro-2-(6-(2-(1-cyclopropyl-1H-pyrazol-4-yl)-6-methylmorpholino)-2,3-dimethyl-4-oxo-3,4-dihydropyrido[3,4-d]pyrimidin-8-yl)benzonitrile

### Step-1—Preparation of 5-chloro-2-(4,4,5,5-tetramethyl-1,3,2-dioxaborolan-2-yl)benzonitrile

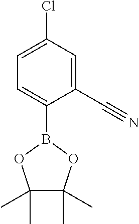 [SMILES: CC1(C)OB(c2ccc(Cl)cc2C#N)OC1(C)C]

To a stirred solution of 2-bromo-5-chlorobenzonitrile (2.5 g, 11.63 mmol) and bispinacolato diborane (6 g, 23.26 mmol) in 1,4-dioxane (30 mL) was added potassium acetate (4.56 g 46.53 mmol) and degassed with argon. PdCl₂(dppf) (0.47 g, 0.58 mmol) was added to the reaction mixture under inert atmosphere. The resulting mixture was heated at 110° C. for 16 h. Reaction mixture was diluted with ethyl acetate, filtered through a pad of celite and washed with ethyl acetate. Combined organic was concentrated under reduced pressure to afford 5-chloro-2-(4,4,5,5-tetramethyl-1,3,2-dioxaborolan-2-yl)benzonitrile (2.5 g, crude) as brown gum.

LCMS Condition C: Rt=2.53 min. m/z 264.4 [M+H]⁺.

### Step-2—Preparation of 5-chloro-2-(6-chloro-2,3-dimethyl-4-oxo-3,4-dihydropyrido[3,4-d]pyrimidin-8-yl)benzonitrile

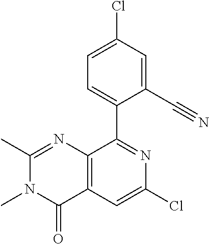 [SMILES: Cc1nc2c(-c3ccc(Cl)cc3C#N)nc(Cl)cc2c(=O)n1C]

To a stirred solution of 8-bromo-6-chloro-2,3-dimethylpyrido[3,4-d]pyrimidin-4 (3H)-one (2 g, 11.63 mmol) and 5-chloro-2-(4,4,5,5-tetramethyl-1,3,2-dioxaborolan-2-yl)benzonitrile (1.6 g, 6.27 mmol) in 1,4-dioxane (50 mL) and water (10 mL) was added K₃PO₄ (2.23 g, 10.45 mmol) and degassed with argon. Pd-118 (454 mg 0.69 mmol) was added to the reaction mixture under inert atmosphere. The resulting mixture was heated at 60° C. for 3 h. Reaction mixture was diluted with ethyl acetate, filtered through a pad of celite. Combined organic was washed with water, brine, dried over sodium sulphate and concentrated under reduced pressure. The crude product was purified by column chromatography (silica gel; 0-40% ethyl acetate-hexane) to afford 5-chloro-2-(6-chloro-2,3-dimethyl-4-oxo-3,4-dihydropyrido[3,4-d]pyrimidin-8-yl)benzonitrile (800 mg, 30.6% yield) as off white solid.

LCMS Condition C: Rt=3.19 min. m/z 345.1 [M+H]⁺.

### Step-3—Preparation of 5-chloro-2-(6-(2-(1-cyclopropyl-1H-pyrazol-4-yl)-6-methylmorpholino)-2,3-dimethyl-4-oxo-3,4-dihydropyrido[3,4-d]pyrimidin-8-yl)benzonitrile [Example 198]

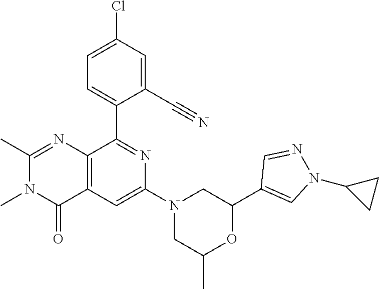 [SMILES: Cc1nc2c(-c3ccc(Cl)cc3C#N)nc(N3CC(C)OC(c4cnn(C5CC5)c4)C3)cc2c(=O)n1C]

To a stirred solution of 5-chloro-2-(6-chloro-2,3-dimethyl-4-oxo-3,4-dihydropyrido[3,4-d]pyrimidin-8-yl)benzonitrile (100 mg, 0.29 mmol) in toluene (3 mL) were added subsequently 2-(1-cyclopropyl-1H-pyrazol-4-yl)-6-methylmorpholine (60 mg, 0.29 mmol), sodium tert-butoxide (55 mg, 0.59 mmol) and Ruphos (13 mg, 0.03 mmol). The resulting mixture was degassed with argon and added Ru-Phos-Pd-G3 (24 mg, 0.03 mmol). The resulting reaction mixture was heated at 70° C. for 4 h. After completion, reaction mixture was filtered through a pad of celite and washed with dichloromethane and concentrated under reduced pressure. Crude product was purified by reverse phase preparative HPLC to afford 5-chloro-2-(6-(2-(1-cyclopropyl-1H-pyrazol-4-yl)-6-methylmorpholino)-2,3-dimethyl-4-oxo-3,4-dihydropyrido[3,4-d]pyrimidin-8-yl)benzonitrile (25 mg, 16% yield) as yellow solid.

Preparative HPLC was done on Waters auto purification instrument. Column name: YMC-Actus C18 (250×20 mm, 5μ) operating at ambient temperature and flow rate of 16 mL/min. Mobile phase: A=20 mM Ammonium Bicarbonate in water, B=Acetonitrile; Gradient Profile: Mobile phase initial composition of 60% A and 40% B, then 40% A and 60% B in 3 min, then to 10% A and 90% B in 20 min., then to 5% A and 95% B in 21 min., held this composition up to 22 min. for column washing, then returned to initial composition in 23 min. and held till 25 min.

¹H-NMR (400 MHz, DMSO-d₆): δ 8.15 (s, 1H), 7.93-7.86 (m, 2H), 7.81 (s, 1H), 7.45 (s, 1H), 7.39 (s, 1H), 4.54 (d, J=10.8 Hz, 1H), 4.41 (d, J=12.8 Hz, 1H), 4.31 (d, J=11.6 Hz, 1H), 3.77-3.59 (m, 2H), 3.53 (s, 3H), 2.91-2.85 (m, 1H), 2.66-2.60 (m, 1H), 2.47 (s, 3H), 1.20 (d, J=6.0 Hz, 3H), 1.01-0.99 (m, 2H), 0.94-0.92 (m, 2H).

LCMS Condition K: Rt=3.20 min. m/z 516.4 [M+H]⁺.

### Example 199 to 203

Example 199[A-B]was synthesized using a similar procedure to Example 118 followed by chiral separation and example 200[A-B] to 203[A-B]were synthesized using similar procedures to example 198 followed by chiral separation.

Chiral separation details of Example 199A-199B to 203A-203B are captured in following table:

| Comp-ound No. | | | Chiral separation |
|---|---|---|---|
| Structure | Name | Starting material | specification |
| Ex. 199A (Peak 1) | 2,3-dimethyl-6- | 2,3-dimethyl-6-(2- | Similar |
| | ((2S,6R)-2-methyl-6- | methyl-6-(2- | specifications as |
| 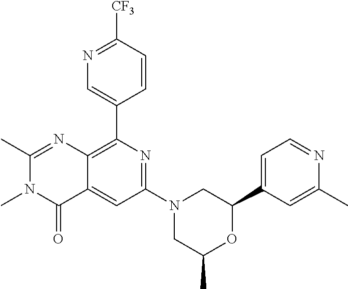 | (2-methylpyridin-4- yl)morpholino)-8-(6- (trifluoromethyl) pyridin-3- yl)pyrido[3,4- d]pyrimidin-4(3H)- one | methylpyridin-4- yl)morpholino)-8-(6- (trifluoromethyl) pyridin-3-yl) pyrido[3,4- d]pyrimidin-4(3H)- one | example-177 Peak 1 Absolute stereochemistry unknown |
| Ex. 199B (Peak 2) | 2,3-dimethyl-6- | | Peak 2 |
| | ((2R,6S)-2-methyl-6- | | Absolute |
| 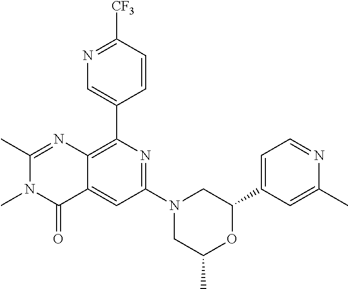 | (2-methylpyridin-4- yl)morpholino)-8-(6- (trifluoromethyl) pyridin-3-yl)pyrido [3,4- d]pyrimidin-4(3H)- one | | stereochemistry unknown |
| Ex. 200A (Peak 1) | 4-(6-((2R,6S)-2-(1- | 4-(6-(2-(1- | Similar |
| | cyclopropyl-1H- | cyclopropyl-1H- | specifications as |
| 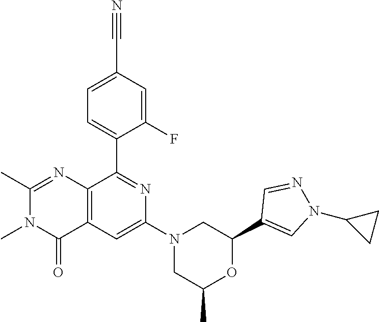 [SMILES: Cc1nc2c(-c3ccc(C#N)cc3F)nc(N3C[C@@H](c4cnn(C5CC5)c4)O[C@@H](C)C3)cc2c(=O)n1C] | pyrazol-4-yl)-6- methylmorpholino)- 2,3-dimethyl-4-oxo- 3,4- dihydropyrido[3,4- d]pyrimidin-8-yl)-3- fluorobenzonitrile | pyrazol-4-yl)-6- methylmorpholino)- 2,3-dimethyl-4-oxo- 3,4- dihydropyrido[3,4- d]pyrimidin-8-yl)-3- fluorobenzonitrile | example-177 Peak 1 Absolute stereochemistry unknown |
| Ex. 200B (Peak 2) | 4-(6-((2S,6R)-2-(1- | | Peak 2 |
| | cyclopropyl-1H- | | Absolute |
| 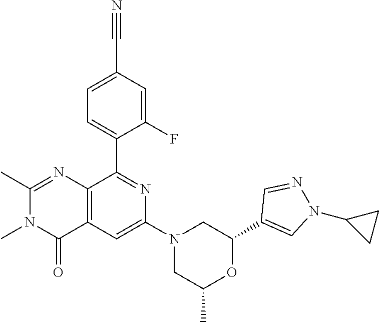 [SMILES: Cc1nc2c(-c3ccc(C#N)cc3F)nc(N3C[C@@H](C)O[C@@H](c4cnn(C5CC5)c4)C3)cc2c(=O)n1C] | pyrazol-4-yl)-6- methylmorpholino)- 2,3-dimethyl-4-oxo- 3,4- dihydropyrido[3,4- d]pyrimidin-8-yl)-3- fluorobenzonitrile | | stereochemistry unknown |
| Ex. 201A (Peak 1) | 2-(6-((2R,6S)-2-(1- | 2-(6-(2-(1- | Similar |
| | cyclopropyl-1H- | cyclopropyl-1H- | specifications as |
| 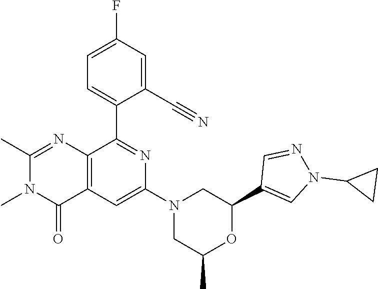 [SMILES: Cc1nc2c(-c3ccc(F)cc3C#N)nc(N3C[C@@H](c4cnn(C5CC5)c4)O[C@@H](C)C3)cc2c(=O)n1C] | pyrazol-4-yl)-6- methylmorpholino)- 2,3-dimethyl-4-oxo- 3,4- dihydropyrido[3,4- d]pyrimidin-8-yl)-5- fluorobenzonitrile | pyrazol-4-yl)-6- methylmorpholino)- 2,3-dimethyl-4-oxo- 3,4- dihydropyrido[3,4- d]pyrimidin-8-yl)-5- fluorobenzonitrile | example-177 Peak 1 Absolute stereochemistry unknown |
| Ex. 201B (Peak 2) | 2-(6-((2S,6R)-2-(1- | | Peak 2 |
| | cyclopropyl-1H- | | Absolute |
| 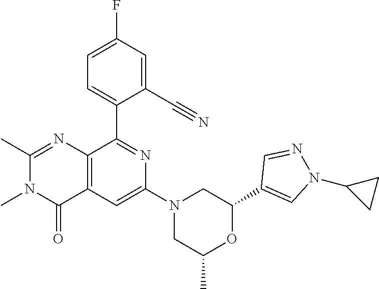 [SMILES: Cc1nc2c(-c3ccc(F)cc3C#N)nc(N3C[C@@H](C)O[C@@H](c4cnn(C5CC5)c4)C3)cc2c(=O)n1C] | pyrazol-4-yl)-6- methylmorpholino)- 2,3-dimethyl-4-oxo- 3,4- dihydropyrido[3,4- d]pyrimidin-8-yl)-5- fluorobenzonitrile | | stereochemistry unknown |
| Ex. 202A (Peak 1) | 2-(6-((2R,6S)-2-(1- | 2-(6-(2-(1- | Similar |
| | cyclopropyl-1H- | cyclopropyl-1H- | specifications as |
| 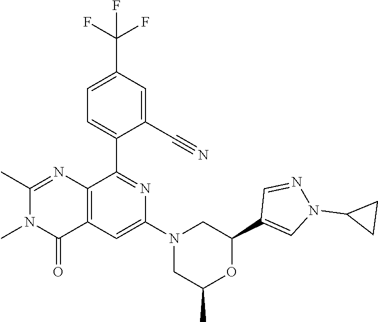 [SMILES: Cc1nc2c(-c3ccc(C(F)(F)F)cc3C#N)nc(N3C[C@@H](c4cnn(C5CC5)c4)O[C@@H](C)C3)cc2c(=O)n1C] | pyrazol-4-yl)-6- methylmorpholino)- 2,3-dimethyl-4-oxo- 3,4- dihydropyrido[3,4- d]pyrimidin-8-yl)-5- (trifluoromethyl) benzonitrile | pyrazol-4-yl)-6- methylmorpholino)- 2,3-dimethyl-4-oxo- 3,4- dihydropyrido[3,4- d]pyrimidin-8-yl)-5- (trifluoromethyl) benzonitrile | example-177 Peak 1 Absolute stereochemistry unknown |
| Ex. 202B (Peak 2) | 2-(6-((2S,6R)-2-(1- | | Peak 2 |
| | cyclopropyl-1H- | | Absolute |
| 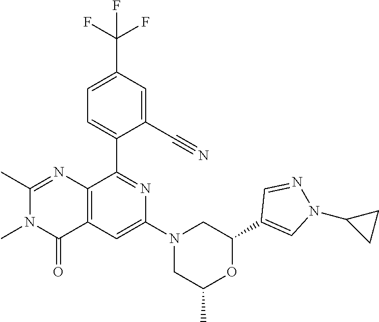 [SMILES: Cc1nc2c(-c3ccc(C(F)(F)F)cc3C#N)nc(N3C[C@@H](C)O[C@@H](c4cnn(C5CC5)c4)C3)cc2c(=O)n1C] | pyrazol-4-yl)-6- methylmorpholino)- 2,3-dimethyl-4-oxo- 3,4- dihydropyrido[3,4- d]pyrimidin-8-yl)-5- (trifluoromethyl) benzonitrile | | stereochemistry unknown |
| Ex. 203A (Peak 1) | (R)-8-(4-chloro-2- | 8-(4-chloro-2- | Similar |
| | fluorophenyl)-2- | fluorophenyl)-2- | specifications as |
| 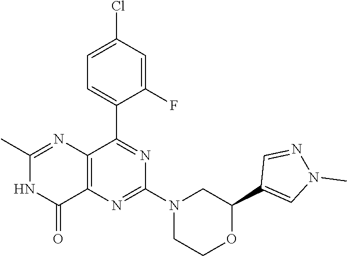 | methyl-6-(2-(1- methyl-1H-pyrazol-4- yl)morpholino) pyrimido[5,4-d] pyrimidin- 4(3H)-one | methyl-6-(2-(1- methyl-1H-pyrazol- 4- yl)morpholino) pyrimido[5,4-d] pyrimidin- 4(3H)-one [corresponding racemate compound synthesized in Example-60] | example-177 Peak 1 Absolute stereochemistry unknown |
| Ex. 203B (Peak 2) | (S)-8-(4-chloro-2- | | Similar |
| | fluorophenyl)-2- | | specifications as |
| 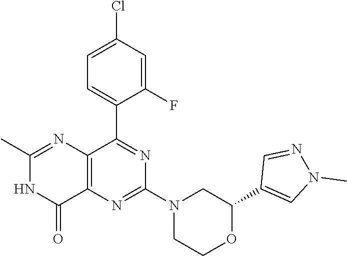 | methyl-6-(2-(1- methyl-1H-pyrazol-4- yl)morpholino) pyrimido[5,4-d] pyrimidin- 4(3H)-one | | example-177 Peak 2 Absolute stereochemistry unknown |

Following table describes analytical data analysis and yield information of examples 199A-199B to examples 203A-203B:

| Compound | | | Analytical | | Yield |
|---|---|---|---|---|---|
| No. | HNMR | LCMS | chiral HPLC | Description | (%) |
| Ex. 199A | ¹H-NMR (400 MHz, MeOD): δ | Condition | CHIRALCEL OX-H | Yellow | 9.8 |
| (Peak 1) | 9.51 (d, J = 1.6 Hz, 1H), 8.85- | K: Rt = | (150 × 4.6) mm, | solid | |
| | 8.83 (dd, J = 8.4 Hz, J = 1.6 Hz, | 2.73 min. | 3μ M.P.- | | |
| | 1H), 8.40 (d, J = 5.2 Hz, 1H), | m/z 511.5 | ACN/MEOH/ | | |
| | 7.91 (d, J = 8.0 Hz, 1H), 7.44- | [M + H]⁺. | IPAMINE | | |
| | 7.43 (m, 2H), 7.36 (d, J = 5.2 Hz, | | (50/50/0.3) | | |
| | 1H), 4.75-4.72 (dd, J = 10.8 Hz, | | Flow Rate - | | |
| | J = 2.8 Hz, 1H), 4.55 (d, J = 12.8 | | 4.0 ml/min | | |
| | Hz, 1H), 4.36 (d, J = 12.0 Hz, | | Solubility: | | |
| | 1H), 3.94-3.91 (m, 1H), 3.62 (s, | | MeOH, Rt = | | |
| | 3H), 2.79-2.72 (m, 2H), 2.59 (s, | | 4.65 min for | | |
| | 3H), 2.56 (s, 3H), 1.37 (d, J = 6.4 | | Peak 1 and Rt = | | |
| | Hz, 3H). | | 3.05 min for | | |
| Ex. 199B | ¹H-NMR (400 MHz, MeOD): δ | Condition | Peak 2. | Yellow | 8.5 |
| (Peak 2) | 9.51 (d, J = 1.6 Hz, 1H), 8.85- | K: Rt = | | solid | |
| | 8.83 (dd, J = 8.4 Hz, J = 1.6 Hz, | 2.73 min. | | | |
| | 1H), 8.40 (d, J = 5.2 Hz, 1H), | m/z 511.5 | | | |
| | 7.91 (d, J = 8.0 Hz, 1H), 7.44- | [M + H]⁺. | | | |
| | 7.43 (m, 2H), 7.36 (d, J = 5.2 Hz, | | | | |
| | 1H), 4.75-4.72 (dd, J = 10.8 Hz, | | | | |
| | J = 2.8 Hz, 1H), 4.55 (d, J = 12.8 | | | | |
| | Hz, 1H), 4.36 (d, J = 12.0 Hz, | | | | |
| | 1H), 3.94-3.91 (m, 1H), 3.62 (s, | | | | |
| | 3H), 2.79-2.72 (m, 2H), 2.59 (s, | | | | |
| | 3H), 2.56 (s, 3H), 1.37 (d, J = 6.4 | | | | |
| | Hz, 3H). | | | | |
| Ex. 200A | ¹H-NMR (400 MHz, DMSO-d₆): | Condition | CHIRALPAK IG | Yellow | 12.2 |
| (Peak 1) | δ 7.94 (d, J = 9.2 Hz, 1H), 7.83 | K: Rt = | (250 × 4.6)mm, | solid | |
| | (s, 1H), 7.81-7.78 (m, 1H), | 2.59 min. | 5μ M.P.- | | |
| | 7.73 (t, J = 7.2 Hz, 1H), 7.46 (s, | m/z 500.5 | Hexane/DCM/ | | |
| | 1H), 7.37 (s, 1H), 4.58-4.55 | [M + H]⁺. | EtOH/IPAmine: | | |
| | (dd, J = 10.4 Hz, J = 2.4 Hz, 1H), | | 50/25/25/0.1 | | |
| | 4.32-4.23 (m, 2H), 3.70-3.67 | | Flow Rate - | | |
| | (m, 2H), 3.51 (s, 3H), 2.85 (t, | | 1.0 ml/min; | | |
| | J = 12.0 Hz, 1H), 2.66-2.56 (m, | | Solubility: | | |
| | 1H), 2.43 (s, 3H), 1.22 (d | | MeOH, Rt = | | |
| | J = 6.4 Hz, 3H), 1.02-0.99 (m, | | 8.57 min for | | |
| | 2H), 0.95-0.92 (m, 2H). | | Peak 1 and Rt = | | |
| Ex. 200B | ¹H-NMR (400 MHz, DMSO-d₆): | Condition | 9.82 min for | Yellow | 8.5 |
| (Peak 2) | δ 7.94 (d, J = 9.2 Hz, 1H), 7.83 | K: Rt = | Peak 2. | solid | |
| | (s, 1H), 7.81-7.78 (m, 1H), | 2.59 min. | | | |
| | 7.73 (t, J = 7.2 Hz, 1H), 7.46 (s, | m/z 500.5 | | | |
| | 1H), 7.37 (s, 1H), 4.58-4.55 | [M + H]⁺. | | | |
| | (dd, J = 10.4 Hz, J = 2.4 Hz, 1H), | | | | |
| | 4.32-4.23 (m, 2H), 3.70-3.67 | | | | |
| | (m, 2H), 3.51 (s, 3H), 2.85 (t, | | | | |
| | J = 12.0 Hz, 1H), 2.66-2.56 (m, | | | | |
| | 1H), 2.43 (s, 3H), 1.22 (d, | | | | |
| | J = 6.4 Hz, 3H), 1.02-0.99 (m, | | | | |
| | 2H), 0.95-0.92 (m, 2H). | | | | |
| Ex. 201A | ¹H-NMR (400 MHz, DMSO-d₆): | Condition | CHIRALPAK IG | Yellow | 38.2 |
| (Peak 1) | δ 7.98-7.93 (m, 2H), 7.82 (s, | K: Rt = | (250 × 4.6)mm, | solid | |
| | 1H), 7.70-7.65 (m, 1H), 7.45 | 3.04 min. | 5μ M.P.- | | |
| | (s, 1H), 7.38 (s, 1H), 4.58-4.54 | m/z 500.5 | ACN/MEOH/ | | |
| | (dd, J = 10.8 Hz, J = 2.4 Hz, 1H), | [M + H]⁺. | IPAMINE | | |
| | 4.41 (d, J = 12.8 Hz, 1H), 4.31 | | (50/50/0.3) | | |
| | (d, J = 12.0 Hz, 1H), 3.78-3.75 | | Flow Rate - | | |
| | (m, 1H), 3.70-3.66 (m, 1H), | | 4.0 ml/min | | |
| | 3.53 (s, 3H), 2.91-2.86 (m, 1H), | | Solubility: | | |
| | 2.66-2.60 (m, 1H), 2.47 (s, 3H), | | MeOH, Rt = | | |
| | 1.20 (d, J = 6.4 Hz, 3H), 1.03- | | 4.04 min for | | |
| | 0.99 (m, 2H), 0.97-0.92 (m, | | Peak 1 and Rt = | | |
| | 2H). | | 4.83 min for | | |
| Ex. 201B | ¹H-NMR (400 MHz, DMSO-d₆): | Condition | Peak 2. | Yellow | 30.8 |
| (Peak 2) | δ 7.98-7.93 (m, 2H), 7.82 (s, | K: Rt = | | solid | |
| | 1H), 7.70-7.65 (m, 1H), 7.45 | 3.04 min. | | | |
| | (s, 1H), 7.38 (s, 1H), 4.58-4.54 | m/z 500.5 | | | |
| | (dd, J = 10.8 Hz, J = 2.4 Hz, 1H), | [M + H]⁺. | | | |
| | 4.41 (d, J = 12.8 Hz, 1H), 4.31 | | | | |
| | (d, J = 12.0 Hz, 1H), 3.78-3.75 | | | | |
| | (m, 1H), 3.70-3.66 (m, 1H), | | | | |
| | 3.53 (s, 3H), 2.91-2.86 (m, 1H), | | | | |
| | 2.66-2.60 (m, 1H), 2.47 (s, 3H), | | | | |
| | 1.20 (d, J = 6.4 Hz, 3H), 1.03- | | | | |
| | 0.99 (m, 2H), 0.97-0.92 (m, | | | | |
| | 2H). | | | | |
| Ex. 202A | ¹H-NMR (400 MHz, DMSO-d₆): | LCMS | CHIRALPAK IG | Yellow | 45.1 |
| (Peak 1) | δ 8.45 (s, 1H), 8.18-8.11(m, | Condition | (250 × 4.6) mm, | solid | |
| | 2H), 7.82 (s, 1H), 7.44 (d, | K: Rt = | 5μ M.P.-0.3% | | |
| | J = 7.6 Hz, 2H), 4.58-4.55 (dd, | 3.27 min. | IPAMINE in | | |
| | J = 10.8 Hz, J = 2.4 Hz, 1H), 4.42 | m/z 550.5 | isopropanol | | |
| | (d, J = 12.4 Hz, 1H), 4.32 (d, | [M + H]⁺. | Flow Rate - | | |
| | J = 12.4 Hz, 1H), 3.77-3.75 (m, | | 4.0 ml/min | | |
| | 1H), 3.69-3.66 (m, 1H), 3.53 (s, | | Solubility: | | |
| | 3H), 2.93-2.89 (m, 1H), 2.67- | | MeOH, Rt = | | |
| | 2.61 (m, 1H), 2.47 (s, 3H), 1.20 | | 3.72 min for | | |
| | (d, J = 6.0 Hz, 3H), 1.01-0.99 (m, | | Peak 1 and Rt = | | |
| | 2H), 0.95-0.92 (m, 2H). | | 4.61 min for | | |
| Ex. 202B | ¹H-NMR (400 MHz, DMSO-d₆): | LCMS | Peak 2. | Yellow | 44.2 |
| (Peak 2) | δ 8.45 (s, 1H), 8.18-8.11(m, | Condition | | solid | |
| | 2H), 7.82 (s, 1H), 7.44 (d, | K: Rt = | | | |
| | J = 7.6 Hz, 2H), 4.58-4.55 (dd, | 3.27 min. | | | |
| | J = 10.8 Hz, J = 2.4 Hz, 1H), 4.42 | m/z 550.5 | | | |
| | (d, J = 12.4 Hz, 1H), 4.32 (d, | [M + H]⁺. | | | |
| | J = 12.4 Hz, 1H), 3.77-3.75 (m, | | | | |
| | 1H), 3.69-3.66 (m, 1H), 3.53 (s, | | | | |
| | 3H), 2.93-2.89 (m, 1H), 2.67- | | | | |
| | 2.61 (m, 1H), 2.47 (s, 3H), 1.20 | | | | |
| | (d, J = 6.0 Hz, 3H), 1.01-0.99 (m, | | | | |
| | 2H), 0.95-0.92 (m, 2H). | | | | |
| Ex. 203A | ¹H-NMR (400 MHz, DMSO-d₆): | LCMS | (R,R) | Yellow | 23.0 |
| (Peak 1) | δ 7.74 (s, 1H), 7.64 (t, J = 8.0 | Condition | WHELK-01 | solid | |
| | Hz, 1H), 7.57 (d, J = 7.2 Hz, | K: Rt = | (250 × 4.6)mm, | | |
| | 1H), 7.42 (d, J = 7.2 Hz, 2H), | 2.61 min. | 5μ M.P.- 0.3% | | |
| | 4.58 (d, J = 13.2 Hz, 1H), 4.51- | m/z 456.29 | IPAMINE in | | |
| | 4.49 (m, 2H), 3.98 (d, J = 10.8 | [M + H]+. | isopropanol | | |
| | Hz, 1H ), 3.81 (s, 3H), 3.65 (t, | | Flow Rate - | | |
| | J = 11.6 Hz, 1H), 3.20-3.10 (m, | | 4.0 ml/min | | |
| | 2H), 2.49 (m, 1H), 2.21 (s, 3H). | | Solubility: | | |
| Ex. 203B | ¹H-NMR (400 MHz, DMSO-d₆): | LCMS | MeOH, Rt = | Yellow | 24.7 |
| (Peak 2) | δ 7.74 (s, 1H), 7.64 (t, J = 8.0 | Condition | 3.72 min for | solid | |
| | Hz, 1H), 7.57 (d, J = 7.2 Hz, | K: Rt = | Peak 1 and Rt = | | |
| | 1H), 7.42 (d, J = 7.2 Hz, 2H), | 2.61 min. | 4.61 min for | | |
| | 4.58 (d, J = 13.2 Hz, 1H), 4.51- | m/z 456.29 | Peak 2. | | |
| | 4.49 (m, 2H), 3.98 (d, J = 10.8 | [M + H]+. | | | |
| | Hz, 1H ), 3.81 (s, 3H), 3.65 (t, | | | | |
| | J = 11.6 Hz, 1H), 3.20-3.10 (m, | | | | |
| | 2H), 2.49 (m, 1H), 2.21 (s, 3H). | | | | |

### Example 204—Biological Evaluation

Human TREM2, in vitro Measurement of Triggering Receptor Expressed on Myeloid Cells 2 activity using cellular phosphorylation of Spleen Tyrosine Kinase (“Syk”) Assay

### Cell Line:

HEK-293 cells were co-transfected with separate plasmids encoding TREM2 and DAP12 to generate a stable cell line. After antibiotic selection, functional clone pool analysis and two successive limiting dilutions, the final clone “HEK293/DAP12+TREM2” underwent a qPCR analysis and a pharmacological validation.

### Assay

TREM2 signaling through DAP12 was monitored in the HEK293/DAP12+TREM2 stable cell line by measuring the phosphorylation levels of the Syk kinase using the commercially available AlphaLISA SureFire Ultra p-SYK (Tyr525/526) Assay Kit (PerkinElmer #ALSU-PSYK), based on the Perkin Elmer AlphaScreen/AlphaLISA technology.

Compounds are transferred to the test plate and tested in full dose response, 8 concentrations in quadruplicate data points. Compound serial dilutions were performed at Cybi-Felix instrument in 100% DMSO and the dose response curves were assembled in automated fashion in 384MPT at Hamilton STARlet instrument. All the stock solutions were prepared at 20 mM in 100% DMSO. For compounds testing, the starting concentration was 100 μM, dilution steps 1:6. A different concentration's range was adapted for compound's activity based on the preliminary results. Finally, a 384MPT reformatted for all compounds at 8 concentrations, quadruplicates, was used as “mother to child” process with a CyBi®-Well dispenser in which 1 μL of each compound was moved into a destination plate pre-filled with 65.6 μL of EMEM cell culture medium (BIOWHITTAKER_cat.BE12-125F), thus obtaining 3× concentrated compounds working solution. In columns 1-2 and 23-24 the control wells were added. In particular, dose response curves of a reference control agonist were included in column 1 and 24 as reference control agonist (Reference control agonists used include Human TREM2 polyclonal Antibody AF1828: R&D Systems; Human TREM2 monoclonal Antibody MAB1828: R&D Systems). The dose response curves were tested starting at 30 μM, dilution step 1:6. Both “source” compound plate and “destination” compound plate were barcoded and a relationship between the two plates was thus generated.

HEK293/DAP12+TREM2 cells were cultured in EMEM medium supplemented with IX Penicillin/Streptomycin (BIOWHITTAKER_cat.DE17-602E), ULTRAGLUTAMINE I 200 mM, 10% Fetal Bovine Serum plus antibiotics referred to as “HEK293 Culture Medium”. The day before the experiment, cells were detached by gentle wash with DPBS, followed by 5 min incubation at 37° C. with Trypsin solution. Cells were then diluted in HEK293 Culture Medium without antibiotics, counted and seeded into 384-well poly-D-Lysine coated microplates black/clear bottom (GREINER 781946) at a density of 10,000 cells/well in 25 μL/well by the use of a MATRIX WellMate dispenser. Plates were placed into a humidified cell culture incubator at 37° C. with 5% CO₂ until the experimental day. 20-24 hours after seeding mature medium was removed and replaced with 10 μL/well of EMEM cell medium supplemented with 0.1% Pluronic F-68 non-ionic surfactant (Thermofisher, 24040032), referred to as “Assay Buffer”, using the CyBi®-Well instrument. Then 5 μL/well of Assay Buffer containing 3× concentrated test compounds or the reference control agonist (in 0.5% final DMSO concentration) were added to the cells with the CyBi®-Well instrument. Cell plates were incubated for 30 min into a humidified cell culture incubator at 37° C. with 5% CO₂·, then the medium was removed by manually discard. 20 μL/well of lysis buffer were dispensed using the CyBi®-Well instrument and plates were incubated for 10 min at room temperature on a plate shaker (350 rpm). Then, 10 μL/well of lysate were transferred to the Alpha plates. The CyBi®-Well instrument was used to dispense 5 μL/well of AlphaLISA Acceptor Bead Solution in IX Immunoassay buffer (Perkin Elmer AL000F). Then the plates were sealed with Heat sealing foil, shaked for 2 minutes (350 rpm) and incubated for 1 hour at room temperature. Following the incubation with the AlphaLISA Acceptor Bead Solution, the CyBi®-Well instrument was used to dispense 5 μL/well of AlphaLISA Donor Bead Solution in IX Immunoassay buffer. The plates were sealed with Heat sealing foil, shaked for 2 minutes (350 rpm) and then incubated for 1 hour at room temperature. At the end of the incubation an AlphaLISA signal was acquired from the donor and acceptor beads using the Pherastar FSX instrument, a high throughput multi-modal microplate reader calibrated to the plate type with the AlphaLISA mirror and filter-set in 384-well mode, 680-615 nanometer excitation wavelength. The total integration time was 0.60 seconds with a 0.30 second excitation time and a gain of 3600.

Data analysis was performed with Genedata Screener® software and reported compounds activity as % effect in relation to the normalization standards. The AlphaScreen Signal was normalized versus Neutral Controls (Assay buffer plus 0.5% DMSO final conc.) and Stimulator Controls (EC₁₀₀ of the reference control agonist in Assay buffer plus 0.5% DMSO final conc.) in order to obtain the Activity[%] for each well. The normalization places the compound activity values on an equivalent scale and makes them comparable across plates or different compound batches. Therefore, the compound activity values were scaled (based on the two references) to a common range (two-point normalization). The following equation was used by the software to normalize the signal values to the desired signal range:

$$N ( x ) = CR + [ ( ( x - < c r > ) / ( < s r > - < c r > ) ) \cdot ( SR - CR ) ]$$

where: x is the signal value of a well; <cr> is the median of the signal values for the Central Reference wells of a plate (median of Neutral Controls); <sr> is the median of the signal values for the Scale Reference wells of a plate (median of Stimulator Controls); CR is the desired median normalized value for the Central Reference (0) and SR is the desired median normalized value for the Scale Reference (100).

The final equation to calculate the Activity % can be simplified as follow:

$$\% Activity = 100 \cdot ( x - < NeutralControls > ) \text{} / ( < StimulatorControls > - < NeutralControls > )$$

where full activation corresponds to % Activity=100.

The fitting of the dose-response curve of each test compound is performed in the Analyzer module of the Screener software on the normalized values and applying the “smart fit” strategy. This strategy allowed an automatic selection between the “Constant Fit” and the “Hill Fit” model calculating which fit model best matched the experimental data. The Constant Fit was applied when no change of activity was detected across the measured concentrations, and the corresponding compounds were further classified as “inactive”. The Hill Fit was applied when the observed activity significantly changed with the compound concentration. In case of Hill Fit, Hill equation was used to determine the concentration at which activity reaches 50% of maximum level, i.e., AC₅₀.

$$Y = {S}_{0} + ( ( {S}_{inf} - {S}_{0} ) / ( 1 + {( 1 {0}^{L o gAC 50} / {10}^{x} )}^{n} ) )$$

where X is Log 10 of compound concentration.

The equation has four parameters:
Zero Activity (S0)—Activity level at zero concentration of test compound;
Infinite Activity (S_{inf})—Activity level at infinite concentration of test compound;
AC₅₀—Concentration at which activity reaches 50% of maximum level. This term corresponds to EC₅₀ in this assay;
Hill coefficient (n)—Measure of the slope at AC₅₀.

The potency of the test compounds was expressed as EC₅₀ corresponding to the test compound concentration able to activate the phospho-Syk AlphaScreen signal to 50% of the maximal response.

The EC₅₀ values measured in this assay for the exemplified compounds is set out in the table below:

| Example | EC₅₀ | Eₘₐₓ |
|---|---|---|
| 1 | C | ++ |
| 1A | D | ++ |
| 1B | C | +++ |
| 2 | B | ++ |
| 3 | B | ++ |
| 4 | D | ++ |
| 5 | C | + |
| 6 | E | ++ |
| 7 | D | + |
| 8 | D | + |
| 9 | D | + |
| 10 | D | ++ |
| 11 | D | ++ |
| 12a | E | ++ |
| 12b | D | ++ |
| 13 | B | ++ |
| 14 | D | ++ |
| 15 | E | + |
| 16 | D | ++ |
| 17 | D | ++ |
| 18 | E | + |
| 19 | C | + |
| 20A | C | ++ |
| 20B | B | ++ |
| 21A | C | ++ |
| 21B | A | ++ |
| 22A | D | ++ |
| 22B | B | ++ |
| 23A | C | + |
| 23B | B | ++ |
| 24A | B | ++ |
| 24B | D | + |
| 25A | C | + |
| 25B | C | + |
| 26A | C | ++ |
| 26B | B | ++ |
| 27A | C | + |
| 27B | C | ++ |
| 28A | E | ++ |
| 28B | C | ++ |
| 29 | D | + |
| 30 | D | ++ |
| 31 | C | ++ |
| 32 | C | + |
| 33 | C | + |
| 34 | D | + |
| 35 | C | + |
| 36 | D | ++ |
| 37 | D | ++ |
| 38A | D | + |
| 39 | C | + |
| 40 | C | ++ |
| 41 | D | ++ |
| 42 | C | + |
| 43 | C | + |
| 44 | D | ++ |
| 45 | C | + |
| 46 | D | + |
| 47 | D | + |
| 48 | C | + |
| 49 | E | + |
| 50 | C | + |
| 51 | E | ++ |
| 52 | D | ++ |
| 53 | D | ++ |
| 54 | E | + |
| 55 | E | + |
| 56 | E | + |
| 57 | E | + |
| 58 | D | ++ |
| 59 | D | ++ |
| 60 | D | + |
| 61 | C | + |
| 62 | B | + |
| 63 | C | + |
| 64 | C | + |
| 65 | C | ++ |
| 66 | E | ++ |
| 67 | D | +++ |
| 68 | E | ++ |
| 69 | C | + |
| 70 | E | + |
| 71A | C | ++ |
| 71B | C | + |
| 72 | D | ++ |
| 73 | D | ++ |
| 74 | E | + |
| 75 | D | + |
| 76 | D | ++ |
| 77 | E | ++ |
| 78 | D | ++ |
| 79 | E | + |
| 80 | E | + |
| 81 | E | + |
| 82 | E | ++ |
| 83 | E | + |
| 84 | D | + |
| 85 | E | + |
| 86 | E | + |
| 87 | E | ++ |
| 88A | E | + |
| 88B | E | + |
| 89A | C | ++ |
| 89B | C | ++ |
| 90 | B | ++ |
| 91 | B | ++ |
| 92 | C | + |
| 93 | C | ++ |
| 94 | B | ++ |
| 95 | D | + |
| 96A | B | ++ |
| 96B | D | + |
| 97A | C | ++ |
| 97B | E | + |
| 98A | C | + |
| 98B | E | + |
| 99 | D | + |
| 100A | C | ++ |
| 100B | D | + |
| 101A | C | ++ |
| 101B | E | + |
| 102A | C | ++ |
| 102B | E | + |
| 103 | B | ++ |
| 104 | D | ++ |
| 105 | C | ++ |
| 106 | D | + |
| 107A | C | ++ |
| 107B | D | ++ |
| 108A | B | ++ |
| 108B | D | + |
| 109A | B | ++ |
| 109B | D | + |
| 110A | B | ++ |
| 110B | C | + |
| 111A | C | ++ |
| 111B | D | + |
| 112A | C | ++ |
| 112B | D | + |
| 113 | B | ++ |
| 114 | B | ++ |
| 115 | B | ++ |
| 116A | D | + |
| 116B | D | + |
| 117A | C | ++ |
| 117B | D | + |
| 118A | C | ++ |
| 118B | E | + |
| 119A | D | + |
| 119B | B | ++ |
| 120A | E | + |
| 120B | C | ++ |
| 121A | D | + |
| 121B | E | + |
| 122A | B | ++ |
| 122B | D | + |
| 123A | B | +++ |
| 123B | C | ++ |
| 124A | C | ++ |
| 124B | B | ++ |
| 125A | C | ++ |
| 125B | C | + |
| 126A | B | ++ |
| 126B | D | + |
| 127A | D | + |
| 127B | E | + |
| 128A | B | +++ |
| 128B | D | ++ |
| 129A | C | ++ |
| 129B | C | + |
| 130A | C | + |
| 130B | D | + |
| 131A | B | ++ |
| 131B | D | + |
| 132A | B | ++ |
| 132B | D | + |
| 133A | C | ++ |
| 133B | D | + |
| 134A | C | ++ |
| 134B | D | + |
| 135A | B | ++ |
| 135B | D | ++ |
| 136A | B | ++ |
| 136B | D | + |
| 137A | B | ++ |
| 137B | D | ++ |
| 138A | C | +++ |
| 138B | D | + |
| 139A | C | ++ |
| 139B | D | + |
| 140A | D | + |
| 140B | C | ++ |
| 141A | C | ++ |
| 141B | C | ++ |
| 142A | D | + |
| 142B | C | ++ |
| 143A | B | ++ |
| 143B | D | + |
| 144A | B | ++ |
| 144B | C | + |
| 145A | B | ++ |
| 145B | C | + |
| 146A | C | ++ |
| 146B | E | + |
| 147A | C | + |
| 147B | D | ++ |
| 148A | E | + |
| 148B | E | + |
| 149A | A | ++ |
| 149B | C | + |
| 150A | C | ++ |
| 150B | E | + |
| 151A | B | ++ |
| 151B | D | + |
| 152A | D | ++ |
| 152B | E | + |
| 153A | B | +++ |
| 153B | D | + |
| 154A | A | ++ |
| 154B | C | ++ |
| 155A | A | ++ |
| 155B | C | ++ |
| 156A | B | ++ |
| 156B | C | ++ |
| 157 | D | + |
| 158 | D | + |
| 159A | B | ++ |
| 159B | D | + |
| 160A | B | ++ |
| 160B | D | + |
| 161A | D | + |
| 161B | B | ++ |
| 162A | B | ++ |
| 162B | C | + |
| 163A | D | + |
| 163B | B | ++ |
| 164A | C | ++ |
| 164B | B | + |
| 165A | B | ++ |
| 165B | C | ++ |
| 166A | B | ++ |
| 166B | C | + |
| 167 | D | + |
| 168A | D | + |
| 168B | B | ++ |
| 169A | A | ++ |
| 169B | C | ++ |
| 170A | D | + |
| 170B | B | ++ |
| 171A | B | +++ |
| 171B | D | + |
| 172A | D | + |
| 172B | B | ++ |
| 173A | C | + |
| 173B | C | + |
| 174A | C | + |
| 174B | D | + |
| 175A | B | ++ |
| 175B | C | ++ |
| 176A | A | ++ |
| 176B | B | ++ |
| 177A | C | + |
| 177B | B | ++ |
| 177C | D | + |
| 178A | B | ++ |
| 178B | B | ++ |
| 179A | B | ++ |
| 179B | A | ++ |
| 180A | E | + |
| 180B | C | + |
| 181A | C | + |
| 181B | B | ++ |
| 182A | B | +++ |
| 182B | D | + |
| 183A | B | ++ |
| 183B | A | ++ |
| 184A | B | ++ |
| 184B | C | + |
| 185 | B | ++ |
| 186A | B | ++ |
| 186B | D | + |
| 187 | C | + |
| 188 | C | + |
| 189A | C | ++ |
| 189B | D | ++ |
| 190A | C | ++ |
| 190B | D | + |
| 191A | A | + |
| 191B | B | ++ |
| 192A | D | + |
| 192B | B | ++ |
| 193A | B | ++ |
| 193B | C | + |
| 194A | C | ++ |
| 194B | D | + |
| 195A | B | + |
| 195B | A | ++ |
| 196A | B | ++ |
| 196B | C | + |
| 197A | C | + |
| 197B | B | + |
| 198 | E | + |
| 199A | C | + |
| 199B | A | ++ |
| 200A | A | ++ |
| 200B | C | ++ |
| 201A | E | + |
| 201B | E | + |
| 202A | E | + |
| 202B | E | + |
| 203A | D | + |
| 203B | D | + |

wherein for EC50 values “A “denotes an EC50 value<1 nM, “B” denotes an EC50 value between 1 nM and 10 nM, “C” denotes an EC50 value between 10 nM and 100 nM, “D” denotes an EC50 value between 100 nM and 1000 nM, “E” denotes an EC50 value greater than 1000 nM; and wherein for Emax values, “+” denotes <75%, “++” denotes 75-110% and “+++” denotes >110% maximal response relative to the maximal response of the reference agonist. For examples 1, 1A and 1B, human TREM2 MAB1828 (vide supra) was used as the reference, whereas example 1B was used for the remaining examples.

### Example 205—Pharmacodynamics and Pharmacokinetics Studies in hTREM2 Knock-In Mice

Humanized TREM2 knockin (hTREM2 KI) mice were used for pharmacodynamics and pharmacokinetics studies. hTREM2 mice were generated by replacement of the mouse TREM2 gene that encodes the full-length protein by the human TREM2 gene in a C57BL/6 mouse strain background. Groups of 10 animals 8-10-week-old hTREM2 mice were treated via oral gavage administration with a TREM2 agonist compound at 50 mg/kg twice a day with a 10-hour interval. After 24 hours, mice were sacrificed and brains were isolated for RNA extraction used for reverse transcription to create complementary DNAs (cDNAs). The relative mRNA expression level of known TREM2 regulated genes were measured by quantitative reverse transcription polymerase chain reaction (RT-qPCR) relative to Glyceraldehyde-3-phosphate dehydrogenase (GAPDH) reference housekeeping gene. Expression of TREM2 regulated genes CXCL10, CCL2, CST7, and TMEM119 were measured as pharmacodynamic biomarkers after TREM2 agonist treatment normalized to vehicle control. These genes are known as microglial activation biomarkers and have previously been reported to respond to TREM2 antibody and small molecule agonist treatment in vivo.

Statistical Analyses. All graphs represent the mean of all samples in each group±SEM.

GraphPad Prism software was used to perform statistical analyses. P<0.05 was considered significant difference between vehicle and TREM2 small molecule treatment determined by T-test.

The relative expression levels for Compound 89A and vehicle were measured.

Treatment with Compound 89A increased brain expression levels of CXCL10 by about 2.5-fold (M=2.554, SD=1.542, T-test p<0.01), of CST7 by 3.25-fold (M=3.25, SD=1.858, T-test p<0.01), of CCL2 by 1.7-fold (M=1.773, SD=0.9372, T-test p<0.05) and of TMEM119 by 1.8-fold (M=1.830, SD=0.5154, T-test p<0.001), as compared to vehicle control.

### REFERENCES

Colonna, M. et al. (2016) Nat Rev Neurosci 17, 201-207
Deczkowska, A. et al. (2020) Perspective, 181, 6, 1207-1217
Hammond, T. R. (2019) Immunity, 50, 4 5955-974
Suárez-Calvet, M. et al. (2016) EMBO Mol Med, 8, 466-476
Yamazaki, K. et al. (2015) Clinical psychopharmacology and neuroscience: the official scientific journal of the Korean College of Neuropsychopharmacology, 13(3), 324-326
Paloneva BM, J. et al. (2001) Neurology, 56 (11) 1552-1558;
Ulrich J. D. et al. (2017) Neuron., 19;94(2):237-248)
Atagi, Y. et al. (2015) J Biol Chem., 290(43), 26043-50
Kleinberger, G. et al (2014) Sci Transl Med., 2,6 (243):243

1. A compound of Formula LXI: 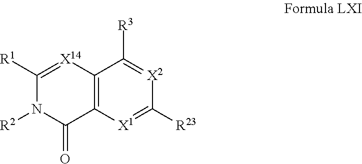
wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₈ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₈ cycloalkyl, C₃₋₈ spiroalkyl, C₅₋₈ tricycloalkyl, C₅₋₈ bicycloalkyl cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl, C₁₋₃ haloalkyl and CN;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XLVI: 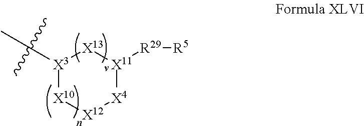
wherein
X³ is N or C(R⁸);
X⁴ is C(R³⁸)(R³⁹), O, NH, NR⁹ or a bond;
X¹⁰ is individually C(R³⁷)(R⁷) or C(O);
X¹¹ is C(R⁴) or N;
X¹² is C(R⁶)(R^{6b});
X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
wherein when R⁶ and R^{6b} are C₁₋₃ alkyl, then R⁶ and R^{6b} are optionally linked together to form a 3-6 membered ring; and/or
wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is individually H, C₁₋₃ alkyl or a bond;
wherein when R⁷ is a bond then R⁶ is C₁₋₃ alkyl, and R⁷ and R⁶ are linked together to form a 3-5 membered ring;
R³⁷ is individually H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
R³⁸ is H or F;
R³⁹ is H or F;
R⁴⁰ is individually H, C₁₋₃ alkyl or a bond;
wherein when R⁴⁰ is a bond then R⁸ is C₁₋₃ alkyl, and R⁸ and R⁴⁰ are linked together to form a 3-5 membered ring;
R⁴¹ is individually H or C₁₋₃ alkyl;
n is 0, 1 or 2; and
v is 0, 1 or 2,
or a pharmaceutically acceptable salt thereof.

2. The compound according to claim 1, wherein the compound is of Formula LXI: 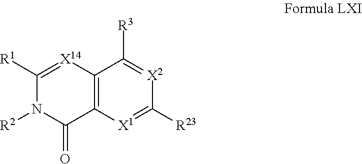
wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₃₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl and C₁₋₃ haloalkyl;
wherein one methylene group of the C₃₋₆ cycloalkyl is optionally replaced with —O— or —N(R²³)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XLVI: 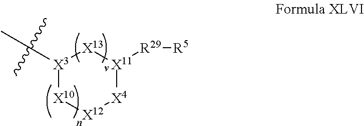
wherein
X³ is N or C(R⁸);
X⁴ is C(R³⁸)(R³⁹), O, NH, NR⁹ or a bond;
X¹⁰ is individually C(R³⁷)(R⁷) or C(O);
X¹¹ is C(R⁴) or N;
X¹² is C(R⁶)(R^{6b});
X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
wherein when R⁶ and R^{6b} are C₁₋₃ alkyl, then R⁶ and R^{6b} are optionally linked together to form a 3-6 membered ring; and/or
wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is individually H or C₁₋₃ alkyl;
R³⁷ is individually H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
R³⁸ is H or F;
R³⁹ is H or F;
R⁴⁰ is individually H, C₁₋₃ alkyl or a bond;
wherein when R⁸ is a bond and R⁴⁰ C₁₋₃ alkyl, then R⁸ and R⁴⁰ are optionally linked together to form a 3-5 membered ring;
R⁴¹ is individually H or C₁₋₃ alkyl;
n is 0, 1 or 2; and
v is 0, 1 or 2,
or a pharmaceutically acceptable salt thereof.

3. The compound according to claim 1, wherein the compound is of Formula LXI: 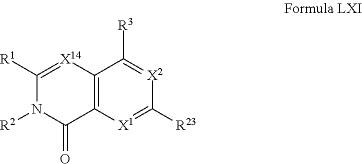
wherein
X¹ is N or C(R⁴²);
R⁴² is H or halogen;
X² is N or CH;
X¹⁴ is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₃₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl and C₁₋₃ haloalkyl;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XLVI: 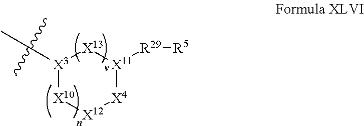
wherein
X³ is N or C(R⁸);
X⁴ is C(R³⁸)(R³⁹), O, NH, NR⁹ or a bond;
X¹⁰ is individually C(R³⁷)(R⁷) or C(O);
X¹¹ is C(R⁴) or N;
X¹² is C(R⁶)(R^{6b});
X¹³ is C(R⁴⁰)(R⁴¹);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
wherein when R⁶ and R^{6b} are C₁₋₃ alkyl, then R⁶ and R^{6b} are optionally linked together to form a 3-6 membered ring; and/or
wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is individually H or C₁₋₃ alkyl;
R³⁷ is individually H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
R³⁸ is H or F;
R³⁹ is H or F;
R⁴⁰ is individually H, C₁₋₃ alkyl or a bond;
wherein when R⁸ is a bond and R⁴⁰ C₁₋₃ alkyl, then R⁸ and R⁴⁰ are optionally linked together to form a 3-5 membered ring;
R⁴¹ is individually H or C₁₋₃ alkyl;
n is 0, 1 or 2; and
v is 1 or 2,
or a pharmaceutically acceptable salt thereof.

4. The compound according to claim 1, wherein the compound is of Formula IX: 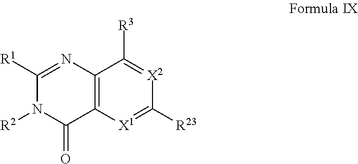
wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with 1 to 3 individually selected substituents R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, 5-membered heterorayl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl or 5-membered heteroaryl is optionally substituted with 1 to 4 substituents independently selected from the group consisting of halogen, C₁₋₃ alkyl and C₁₋₃ haloalkyl;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl, C₃₋₆ cycloalkyl and halogen;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XX: 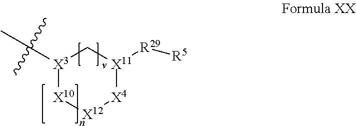
wherein
X³ is N or C(R⁸);
X⁴ is CH₂, O, CHF, CF₂, NH, NR⁹ or a bond;
X¹⁰ is C(H)(R⁷) or C(O);
X¹¹ is C(R⁴) or N;
X¹² is C(R⁶)(R^{6b});
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents independently selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
wherein when R⁶ and R^{6b} are C₁₋₃ alkyl, then R⁶ and R^{6b} are optionally linked together to form a 3-6 membered ring; and/or
wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
n is 0, 1 or 2; and
v is 1 or 2,
or a pharmaceutically acceptable salt thereof.

5. The compound according to claim 1, wherein the compound is of Formula IX: 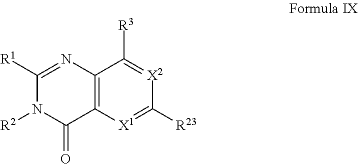
wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₈ cycloalkyl is optionally substituted with R¹⁰;
R^{a} is H or C₁₋₆ alkyl;
R^{b} is H or C₁₋₆ alkyl;
R² is selected from the group consisting of C₁₋₈ alkyl, C₃₋₈ cycloalkyl and H, wherein the C₁-6 alkyl or C₃-6 cycloalkyl is optionally substituted with R¹¹;
R³ is selected from the group consisting of C₁₋₈ alkyl, C₁₋₈ haloalkyl, C₃₋₈ cycloalkyl, C₃₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
wherein one methylene group of the C₃₋₆ cycloalkyl is optionally replaced with —O— or —N(R²⁸);
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl and C₃₋₆ cycloalkyl;
R¹¹ is selected from the group consisting of —O—C₁₋₆ alkyl, C₃₋₆ cycloalkyl, C₁₋₆ haloalkyl and phenyl, wherein the phenyl is optionally substituted with 1 to 5 substituents individually selected from —O—C₁₋₆ alkyl;
R²³ is of Formula XX: 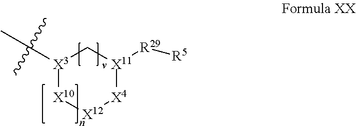
wherein
X³ is N or C(R⁸);
X⁴ is CH₂, O, CHF, CF₂, NH, NR⁹ or a bond;
X¹⁰ is C(H)(R⁷) or C(O);
X¹¹ is C(R⁴) or N;
X¹² is C(R⁶)(R^{6b});
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents independently selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H, halogen or C₁₋₃ alkyl;
R^{6b} is H, halogen or C₁₋₃ alkyl;
wherein when R⁶ and R^{6b} are C₁₋₃ alkyl, then R⁶ and R^{6b} are optionally linked together to form a 3-6 membered ring; and/or
wherein when R⁶ and R^{6b} are C₁₋₃ alkyl, then one methylene group is optionally replaced with —O— or —N(R³⁵)—, wherein R³⁵ is selected from the group consisting of H, C₁₋₃ alkyl and —C(O)C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
n is 0, 1 or 2; and
v is 1 or 2
or a pharmaceutically acceptable salt thereof.

6. The compound according to claim 1, wherein the compound is of Formula IX: 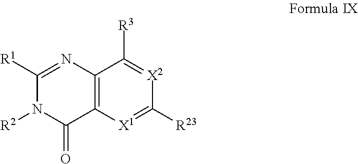
wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₈ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₈ cycloalkyl, C₃₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl and C₃₋₈ cycloalkyl;
R¹¹ is selected from the group consisting of —O—C₁₋₆ alkyl, C₃₋₆ cycloalkyl and C₁₋₆ haloalkyl;
R²³ is of Formula XVI: 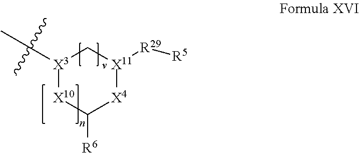
wherein
X³ is N or C(R⁸);
X⁴ is CH₂, O, CHF, CF₂, NH, NR⁹ or a bond;
X¹⁰ is C(H)(R⁷) or C(O);
X¹¹ is C(R⁴) or N;
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents independently selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O), —O—C₁₋₃ alkyl and —N(R³⁰)(R³¹);
R⁶ is H or C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl, —C(O)—C₃₋₆ cycloalkyl or C₁₋₆ haloalkyl, wherein the C₁₋₆ alkyl, —C(O)—C₁₋₆ alkyl or —C(O)—C₃₋₆ cycloalkyl is optionally substituted with 1 to 3 substituents independently selected from halogen and C₁₋₃ alkoxy;
R²⁴ is C₁₋₆ alkyl or aryl, wherein the C₁₋₆ alkyl is optionally substituted with C₁₋₃ alkoxy or halogen;
R²⁹ is a bond, —O— or C₁₋₆ hydrocarbon chain wherein one methylene group is optionally replaced with —O—;
R³⁰ is H or C₁₋₃ alkyl;
R³¹ is H or C₁₋₃ alkyl;
n is 0, 1 or 2; and
v is 1 or 2
or a pharmaceutically acceptable salt thereof.

7. The compound according to claim 1, wherein the compound is of Formula IX: 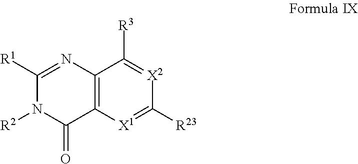
wherein
X¹ is N or CH;
X² is N or CH;
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₃₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is individually selected from the group consisting of —O—C₁₋₆ alkyl and C₃₋₆ cycloalkyl;
R¹¹ is selected from the group consisting of —O—C₁₋₆ alkyl, C₃₋₆ cycloalkyl and C₁₋₆ haloalkyl;
R²³ is of Formula X: 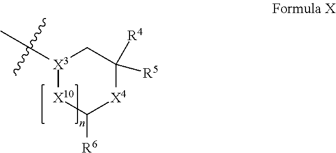
wherein
X³ is N or C(R⁸);
X⁴ is CH₂, O, CHF, CF₂, NH, NR⁹ or a bond;
X¹⁰ is C(H)(R⁷) or C(O);
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with oxo (═O), and
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents independently selected from the group consisting of halogen, C₁₋₃ alkyl and —C(O)O(C₁₋₆ alkyl);
(c) the C₁₋₆ alkyl is optionally substituted with —N(H)C(O)R²⁴;
R⁶ is H or C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₁₋₆ alkyl or —C(O)—C₃₋₆ cycloalkyl;
R²⁴ is C₁₋₆ alkyl or aryl, wherein the C₁₋₆ alkyl is optionally substituted with C₁₋₃ alkoxy or halogen; and
n is 0, 1 or 2,
or a pharmaceutically acceptable salt thereof.

8. The compound according to any one of the preceding claims, wherein the compound is of Formula I: 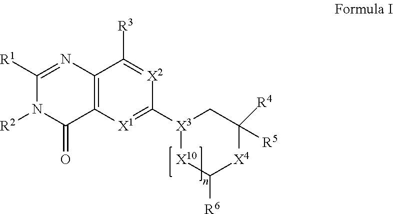
wherein
X¹ is N or CH;
X² is N or CH;
X³ is N or C(R⁸);
X⁴ is CH₂, O, CHF, CF₂, NH, NR⁹ or a bond;
X¹⁰ is C(H)(R⁷) or C(O);
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₃₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl,
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, 5-membered heteroaryl and 6-membered heteroaryl, wherein
(a) the C₃₋₆ cycloalkyl or C₃₋₆ heterocycloalkyl is optionally substituted with oxo (═O), and
(b) the phenyl, 5-membered heteroaryl or 6-membered heteroaryl is optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents independently selected from the group consisting of halogen, C₁₋₃ alkyl and —C(O)O(C₁₋₆ alkyl);
R⁶ is H or C₁₋₃ alkyl;
R⁷ is H or C₁₋₃ alkyl;
R⁸ is selected from the group consisting of H, —OH, —O-alkyl and halogen;
R⁹ is C₁₋₆ alkyl, —C(O)—C₃₋₆ cycloalkyl, —C(O)—C₁₋₆ alkyl, C₁₋₆ alkyl-O— C₁₋₆ alkyl or C₁₋₆ haloalkyl;
R¹⁰ is selected from the group consisting of —O—C₁₋₆ alkyl and C₃₋₆ cycloalkyl;
R¹¹ is selected from the group consisting of —O—C₁₋₆ alkyl and C₃₋₆ cycloalkyl;
n is 0, 1 or 2,
or a pharmaceutically acceptable salt thereof.

9. The compound according to any one of the preceding claims, wherein R²³ is of Formula Xa: 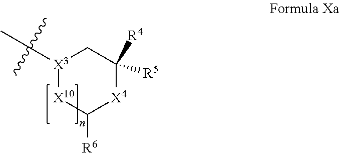
or a pharmaceutically acceptable salt thereof.

10. The compound according to any one of the preceding claims, wherein R²³ is of Formula Xb: 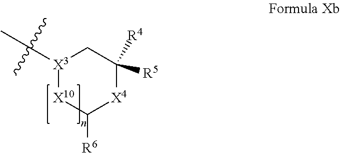
or a pharmaceutically acceptable salt thereof.

11. The compound according to any one of the preceding claims, wherein R²³ is of Formula XXa: 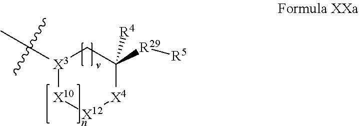
or a pharmaceutically acceptable salt thereof.

12. The compound according to any one of the preceding claims, wherein R²³ is of Formula XXb: 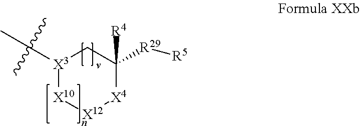
or a pharmaceutically acceptable salt thereof.

13. The compound according to any one of the preceding claims, wherein R²³ is of Formula XXc: 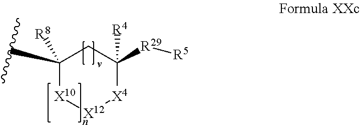
or a pharmaceutically acceptable salt thereof.

14. The compound according to any one of the preceding claims, wherein R²³ is of Formula XXd: 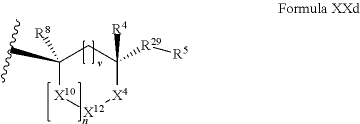
or a pharmaceutically acceptable salt thereof.

15. I The compound according to any one of the preceding claims, wherein R²³ is of Formula XXe: 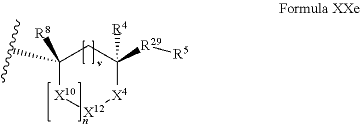
or a pharmaceutically acceptable salt thereof.

16. The compound according to any one of the preceding claims, wherein R²³ is of Formula XXf: 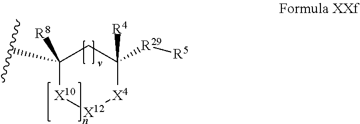
or a pharmaceutically acceptable salt thereof.

17. The compound according to any one of the preceding claims, wherein the compound is of Formula Ia or Formula Ib: 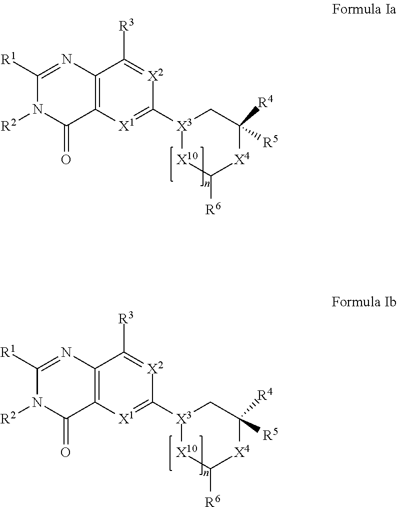
or a pharmaceutically acceptable salt thereof.

18. The compound according to any one of the preceding claims, wherein X¹ is N, or a pharmaceutically acceptable salt thereof.

19. The compound according to any one of the preceding claims, wherein X¹ is C(R⁴²), or a pharmaceutically acceptable salt thereof.

20. The compound according to any one of the preceding claims, wherein R⁴² is H,
or a pharmaceutically acceptable salt thereof.

21. The compound according to any one of the preceding claims, wherein R⁴² is halogen, or a pharmaceutically acceptable salt thereof.

22. The compound according to any one of the preceding claims, wherein R⁴² is F, or a pharmaceutically acceptable salt thereof.

23. The compound according to any one of the preceding claims, wherein X¹ is CH, or a pharmaceutically acceptable salt thereof.

24. The compound according to any one of the preceding claims, wherein X¹ is CF, or a pharmaceutically acceptable salt thereof.

25. The compound according to any one of the preceding claims, wherein X² is N, or a pharmaceutically acceptable salt thereof.

26. The compound according to any one of the preceding claims, wherein X² is CH, or a pharmaceutically acceptable salt thereof.

27. The compound according to any one of the preceding claims, wherein X¹⁴ is N, or a pharmaceutically acceptable salt thereof.

28. The compound according to any one of the preceding claims, wherein X¹⁴ is CH, or a pharmaceutically acceptable salt thereof.

29. The compound according to any one of the preceding claims, wherein X¹ is N and X² is N, or a pharmaceutically acceptable salt thereof.

30. The compound according to any one of the preceding claims, wherein X¹ is N and X² is CH, or a pharmaceutically acceptable salt thereof.

31. The compound according to any one of the preceding claims, wherein X¹ is CH and X² is N, or a pharmaceutically acceptable salt thereof.

32. The compound according to any one of the preceding claims, wherein X¹ is CH and X² is CH, or a pharmaceutically acceptable salt thereof.

33. The compound according to any one of the preceding claims, wherein X¹ is N, X² is N and X¹⁴ is N, or a pharmaceutically acceptable salt thereof.

34. The compound according to any one of the preceding claims, wherein X¹ is N, X² is N and X¹⁴ is CH, or a pharmaceutically acceptable salt thereof.

35. The compound according to any one of the preceding claims, wherein X¹ is N, X² is CH and X¹⁴ is N, or a pharmaceutically acceptable salt thereof.

36. The compound according to any one of the preceding claims, wherein X¹ is CH, X² is N and X¹⁴ is N, or a pharmaceutically acceptable salt thereof.

37. The compound according to any one of the preceding claims, wherein X¹ is CH, X² is CH and X¹⁴ is N, or a pharmaceutically acceptable salt thereof.

38. The compound according to any one of the preceding claims, wherein X¹ is CH, X² is N and X¹⁴ is CH, or a pharmaceutically acceptable salt thereof.

39. The compound according to any one of the preceding claims, wherein X¹ is N, X² is CH and X¹⁴ is CH, or a pharmaceutically acceptable salt thereof.

40. The compound according to any one of the preceding claims, wherein X¹ is CH, X² is CH and X¹⁴ is CH, or a pharmaceutically acceptable salt thereof.

41. The compound according to any one of the preceding claims, wherein X¹ is CF, X² is N and X¹⁴ is N, or a pharmaceutically acceptable salt thereof.

42. The compound according to any one of the preceding claims, wherein the compound is of Formula II: 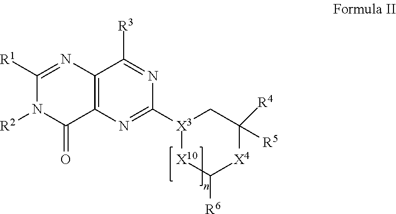
or a pharmaceutically acceptable salt thereof.

43. The compound according to any one of the preceding claims, wherein the compound is of Formula II: 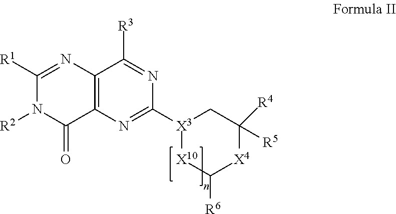
wherein
R¹ is selected from the group consisting of C₁₋₆ alkyl, C₃₋₆ cycloalkyl, H, and NR^{a}R^{b}, wherein the C₁₋₆ alkyl or C₃₋₆ cycloalkyl is optionally substituted with R¹⁰;
R^{a} is H or C₁₋₆ alkyl;
R^{b} is H or C₁₋₆ alkyl;
R² is selected from the group consisting of C₁₋₆ alkyl, C₃₋₆ cycloalkyl and H, wherein the C₁₋₆ alkyl or C₃₋₆ cycloalkyl is optionally substituted with R¹¹;
R³ is selected from the group consisting of C₁₋₆ alkyl, C₁₋₆ haloalkyl, C₃₋₆ cycloalkyl, C₅₋₈ spiroalkyl, C₅₋₈ tricycloalkyl, cyclopent-1-en-1-yl, cyclohex-1-en-1-yl, phenyl, 6-membered heteroaryl, azetidine-1-yl, pyrrolidine-1-yl, 3-azabicyclo[3.1.0]hexan-3-yl, piperidine-1-yl, and —OCH₂—(C₃₋₆ cycloalkyl),
wherein the C₁₋₆ alkyl, C₃₋₆ cycloalkyl, C₅₋₈ spiroalkyl, C₅₋₈ tricycloalkyl, cyclopent-1-en-1-yl, cyclohex-1-en-1-yl, phenyl or 6-membered heteroaryl is optionally substituted with 1 to 4 substituents independently selected from the group consisting of halogen, C₁₋₃ alkyl and C₁₋₃ haloalkyl;
wherein one methylene group of the C₃₋₆ cycloalkyl is optionally replaced with —O— or —N(R²⁸)—;
R²⁸ is selected from the group consisting of H, C₁₋₃ alkyl and —C(O)C₁₋₃ alkyl;
and wherein the azetidine-1-yl, pyrrolidine-1-yl, 3-azabicyclo[3.1.0]hexan-3-yl, piperidine-1-yl, or —OCH₂—(C₃₋₆ cycloalkyl) is optionally substituted with 1 to 4 substituents independently selected from the group consisting of halogen, C₁₋₃ alkyl, C₁₋₃ haloalkyl, C₁₋₃ alkoxy and C₁₋₃ haloalkoxy;
R¹⁰ is selected from the group consisting of —O—C₁₋₆ alkyl and C₃₋₆ cycloalkyl;
R¹¹ is selected from the group consisting of —O—C₁₋₆ alkyl, C₃₋₆ cycloalkyl and C₁₋₆ haloalkyl;
X³ is N or C(R⁸);
X⁴ is CH₂, O, CHF, CF₂, NH, NR⁹ or a bond;
X¹⁰ is CH₂;
R⁴ is H or C₁₋₃ alkyl;
R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with oxo (═O), and
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
(c) the C₁₋₆ alkyl is optionally substituted with —N(H)C(O)R²⁴, -oxo (═O), —O—C₁₋₃ alkyl and —N(R³⁰)(R³¹);
R⁶ is H or C₁₋₃ alkyl;
R⁸ is selected from the group consisting of H, —OH, —O-alkyl and halogen;
R⁹ is C₁₋₆ alkyl, —C(O)—C₁₋₆ alkyl or —C(O)—C₃₋₆ cycloalkyl;
R²⁴ is C₁₋₆ alkyl or aryl, wherein the C₁₋₆ alkyl is optionally substituted with C₁₋₃ alkoxy or halogen; and
n is 1,
or a pharmaceutically acceptable salt thereof.

44. The compound according to any one of the preceding claims, wherein R¹ is C₁₋₆ alkyl optionally substituted with R¹⁰, or a pharmaceutically acceptable salt thereof.

45. The compound according to any one of the preceding claims, wherein R¹ is C₁₋₃ alkyl optionally substituted with R¹⁰, or a pharmaceutically acceptable salt thereof.

46. The compound according to any one of the preceding claims, wherein R¹ is —CH₃, or a pharmaceutically acceptable salt thereof.

47. The compound according to any one of the preceding claims, wherein R¹ is C₁₋₆ alkyl optionally substituted with 1 to 3 individually selected substituents R¹⁰, or a pharmaceutically acceptable salt thereof.

48. The compound according to any one of the preceding claims, wherein R¹ is C₁₋₃ alkyl optionally substituted with 1 to 3 individually selected substituents R¹⁰, or a pharmaceutically acceptable salt thereof.

49. The compound according to any one of the preceding claims, wherein R¹⁰ is halogen, such as F, or a pharmaceutically acceptable salt thereof.

50. The compound according to any one of the preceding claims, wherein R¹ is —CF₃, or a pharmaceutically acceptable salt thereof.

51. The compound according to any one of the preceding claims, wherein R¹ is C₃₋₆ cycloalkyl optionally substituted with R¹⁰, or a pharmaceutically acceptable salt thereof.

52. The compound according to any one of the preceding claims, wherein R¹ is H, or a pharmaceutically acceptable salt thereof.

53. The compound according to any one of the preceding claims, wherein R² is C₁₋₆ alkyl optionally substituted with R¹¹, or a pharmaceutically acceptable salt thereof.

54. The compound according to any one of the preceding claims, wherein R² is C₁₋₃ alkyl optionally substituted with R¹¹, or a pharmaceutically acceptable salt thereof.

55. The compound according to any one of the preceding claims, wherein R² is —CH₃, or a pharmaceutically acceptable salt thereof.

56. The compound according to any one of the preceding claims, wherein R² is H.

57. The compound according to any one of the preceding claims, wherein R² is C₃₋₆ cycloalkyl optionally substituted with R¹¹, or a pharmaceutically acceptable salt thereof.

58. The compound according to any one of the preceding claims, wherein R¹¹ is —O—C₁₋₆ alkyl, such as —O—CH₃, or a pharmaceutically acceptable salt thereof.

59. The compound according to any one of the preceding claims, wherein R² is C₁₋₃ alkyl substituted with R¹¹, and R¹¹ is —O—C₁₋₆ alkyl, or a pharmaceutically acceptable salt thereof.

60. The compound according to any one of the preceding claims, wherein R² is —CH₂CH₂OCH₃, or a pharmaceutically acceptable salt thereof.

61. The compound according to any one of the preceding claims, wherein R² is C₁₋₆ alkyl substituted with phenyl optionally substituted with —O—C₁₋₆ alkyl, or a pharmaceutically acceptable salt thereof.

62. The compound according to any one of the preceding claims, wherein R² is C, alkyl substituted with phenyl substituted with —O—C₁₋₆ alkyl, such as —O—C₁₋₃ alkyl, for example —O—C₁ alkyl or a pharmaceutically acceptable salt thereof.

63. The compound according to any one of the preceding claims, wherein R³ is phenyl optionally substituted with 1 to 4 substituents independently selected from the group consisting of halogen, C₁₋₃ alkyl, C₁₋₃ haloalkyl and CN, or a pharmaceutically acceptable salt thereof.

64. The compound according to any one of the preceding claims, wherein R³ is phenyl optionally substituted with 1 to 4 substituents independently selected from the group consisting of halogen, C₁₋₃ alkyl and C₁₋₃ haloalkyl, or a pharmaceutically acceptable salt thereof.

65. The compound according to any one of the preceding claims, wherein R³ is of Formula III: 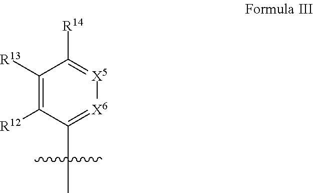
wherein
X⁵ is C(R¹⁵) or N;
X⁶ is CH or N;
R¹² is H, halogen, C₁₋₃ alkyl, C₁₋₃ haloalkyl or CN;
R¹³ is H, halogen, C₁₋₃ alkyl, C₁₋₃ haloalkyl or CN;
R¹⁴ is H, halogen, C₁₋₃ alkyl, C₁₋₃ haloalkyl or CN; and
R¹⁵ is H, halogen, C₁₋₃ alkyl, C₁₋₃ haloalkyl or CN,
or a pharmaceutically acceptable salt thereof.

66. The compound according to any one of the preceding claims, wherein R³ is of Formula III: 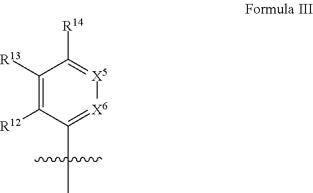
wherein
X⁵ is C(R¹⁵) or N;
X⁶ is CH or N;
R¹² is H, halogen, C₁₋₃ alkyl or C₁₋₃ haloalkyl;
R¹³ is H, halogen, C₁₋₃ alkyl or C₁₋₃ haloalkyl;
R¹⁴ is H, halogen, C₁₋₃ alkyl or C₁₋₃ haloalkyl; and
R¹⁵ is H, halogen, C₁₋₃ alkyl or C₁₋₃ haloalkyl,
or a pharmaceutically acceptable salt thereof.

67. The compound according to any one of the preceding claims, wherein
X⁵ is C(R¹⁵) or N;
X⁶ is CH;
R¹² is H or halogen;
R¹³ is H or halogen;
R¹⁴ is H, halogen, or C₁₋₃ haloalkyl; and
R¹⁵ is H or halogen,
or a pharmaceutically acceptable salt thereof.

68. The compound according to any one of the preceding claims, wherein X⁵ is N and X⁶ is CH, or a pharmaceutically acceptable salt thereof.

69. The compound according to any one of the preceding claims, wherein X⁵ is CH and X⁶ is N.

70. The compound according to any one of the preceding claims, wherein R³ is of Formula IV: 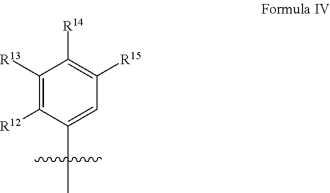
wherein
R¹² is H, halogen, C₁₋₃ alkyl, C₁₋₃ haloalkyl or CN;
R¹³ is H, halogen, C₁₋₃ alkyl, C₁₋₃ haloalkyl or CN;
R¹⁴ is H, halogen, C₁₋₃ alkyl, C₁₋₃ haloalkyl or CN; and
R¹⁵ is H, halogen, C₁₋₃ alkyl, C₁₋₃ haloalkyl or CN,
or a pharmaceutically acceptable salt thereof.

71. The compound according to any one of the preceding claims, wherein R³ is of Formula IV: 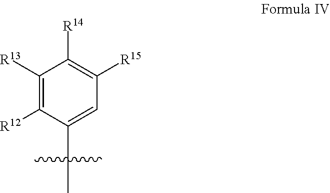
wherein
R¹² is H, halogen, C₁₋₃ alkyl or C₁₋₃ haloalkyl;
R¹³ is H, halogen, C₁₋₃ alkyl or C₁₋₃ haloalkyl;
R¹⁴ is H, halogen, C₁₋₃ alkyl or C₁₋₃ haloalkyl; and
R¹⁵ is H, halogen, C₁₋₃ alkyl or C₁₋₃ haloalkyl,
or a pharmaceutically acceptable salt thereof.

72. The compound according to any one of the preceding claims, wherein R¹² is H, or a pharmaceutically acceptable salt thereof.

73. The compound according to any one of the preceding claims, wherein R¹² is halogen, such as F or Cl, or a pharmaceutically acceptable salt thereof.

74. The compound according to any one of the preceding claims, wherein R¹² is C₁₋₃ alkyl, such as —CH₃, or a pharmaceutically acceptable salt thereof.

75. The compound according to any one of the preceding claims, wherein R¹² is CN, or a pharmaceutically acceptable salt thereof.

76. The compound according to any one of the preceding claims, wherein R¹³ is H, or a pharmaceutically acceptable salt thereof.

77. The compound according to any one of the preceding claims, wherein R¹³ is halogen, such as F, or a pharmaceutically acceptable salt thereof.

78. The compound according to any one of the preceding claims, wherein R¹⁴ is halogen, such as F or C, or a pharmaceutically acceptable salt thereof.

79. The compound according to any one of the preceding claims, wherein R¹⁴ is C₁₋₃ alkyl, such as —CH₃, or a pharmaceutically acceptable salt thereof.

80. The compound according to any one of the preceding claims, wherein R¹⁴ is C₁₋₃ haloalkyl, such as —CF₃, or a pharmaceutically acceptable salt thereof.

81. The compound according to any one of the preceding claims, wherein R¹⁴ is C₁₋₃ haloalkyl, such as —CHF₂, or a pharmaceutically acceptable salt thereof.

82. The compound according to any one of the preceding claims, wherein R¹⁴ is CN, or a pharmaceutically acceptable salt thereof.

83. The compound according to any one of the preceding claims, wherein R¹⁵ is H, or a pharmaceutically acceptable salt thereof.

84. The compound according to any one of the preceding claims, wherein R¹⁵ is halogen, such as F, or a pharmaceutically acceptable salt thereof.

85. The compound according to any one of the preceding claims, or a pharmaceutically acceptable salt thereof, wherein R³ is selected from the group consisting of:

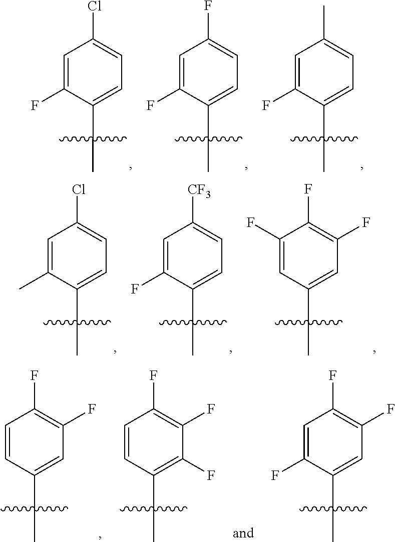

86. The compound according to any one of the preceding claims, or a pharmaceutically acceptable salt thereof, wherein R³ is

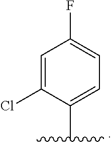 [SMILES: *c1ccc(F)cc1Cl]

87. The compound according to any one of the preceding claims, or a pharmaceutically acceptable salt thereof, wherein R³ is selected from the group consisting of:

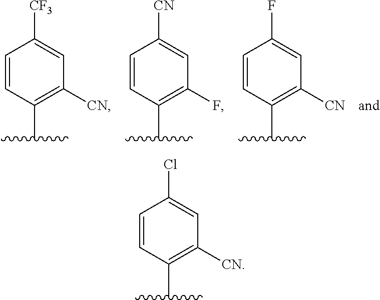

88. The compound according to any one of the preceding claims, or a pharmaceutically acceptable salt thereof, wherein R³ is

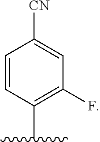 [SMILES: *c1ccc(C#N)cc1F]

89. The compound according to any one of the preceding claims, or a pharmaceutically acceptable salt thereof, wherein R³ is selected from the group consisting of:

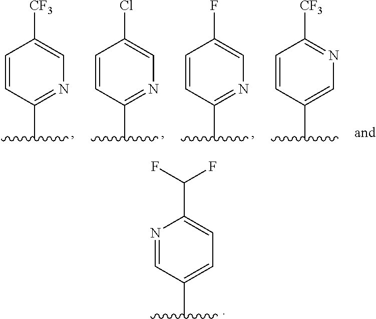

90. The compound according to any one of the preceding claims, wherein R³ is a 5-membered heteroaryl optionally substituted with 1 to 4 substituents independently selected from the group consisting of halogen, C₁₋₃ alkyl and C₁₋₃ haloalkyl,
or a pharmaceutically acceptable salt thereof.

91. The compound according to any one of the preceding claims, wherein R³ is a 5-membered heteroaryl optionally substituted with 1 to 3 substituents independently selected from the group consisting of halogen, C₁₋₃ alkyl and C₁₋₃ haloalkyl, or a pharmaceutically acceptable salt thereof.

92. The compound according to any one of the preceding claims, wherein R³ is a pyrazolyl optionally substituted with 1 to 3 substituents independently selected from the group consisting of halogen, C₁₋₃ alkyl and C₁₋₃ haloalkyl, or a pharmaceutically acceptable salt thereof.

93. The compound according to any one of the preceding claims, wherein R³ is 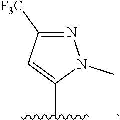 [SMILES: *c1cc(C(F)(F)F)nn1C]
or a pharmaceutically acceptable salt thereof.

94. The compound according to any one of the preceding claims, wherein R³ is C₃₋₆ cycloalkyl optionally substituted with 1 to 4 substituents independently selected from the group consisting of halogen, C₁₋₃ alkyl and C₁₋₃ haloalkyl, or a pharmaceutically acceptable salt thereof.

95. The compound according to any one of the preceding claims, wherein R³ is of Formula V: 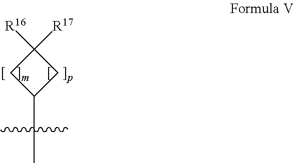
wherein
R¹⁶ is H, halogen, C₁₋₃ alkyl and C₁₋₃ haloalkyl;
R¹⁷ is H, halogen, C₁₋₃ alkyl and C₁₋₃ haloalkyl;
m is 1, 2 or 3; and
p is 1, 2 or 3,
or a pharmaceutically acceptable salt thereof.

96. The compound according to any one of the preceding claims, wherein m is 1, or a pharmaceutically acceptable salt thereof.

97. The compound according to any one of the preceding claims, wherein m is 2, or a pharmaceutically acceptable salt thereof.

98. The compound according to any one of the preceding claims, wherein p is 1, or a pharmaceutically acceptable salt thereof.

99. The compound according to any one of the preceding claims, wherein p is 2, or a pharmaceutically acceptable salt thereof.

100. The compound according to any one of the preceding claims, wherein R¹⁶ is H, or a pharmaceutically acceptable salt thereof.

101. The compound according to any one of the preceding claims, wherein R¹⁶ is halogen, such as F, or a pharmaceutically acceptable salt thereof.

102. The compound according to any one of the preceding claims, wherein R¹⁶ is C₁₋₃ alkyl, such as —CH₃, or a pharmaceutically acceptable salt thereof.

103. The compound according to any one of the preceding claims, wherein R¹⁶ is C₁₋₃ haloalkyl, such as —CF₃ or —CHF₂, or a pharmaceutically acceptable salt thereof.

104. The compound according to any one of the preceding claims, wherein R¹⁷ is H, or a pharmaceutically acceptable salt thereof.

105. The compound according to any one of the preceding claims, wherein R¹⁷ is halogen, such as F, or a pharmaceutically acceptable salt thereof.

106. The compound according to any one of the preceding claims, wherein R¹⁷ is C₁₋₃ alkyl, such as —CH₃, or a pharmaceutically acceptable salt thereof.

107. The compound according to any one of the preceding claims, wherein R¹⁷ is C₁₋₃ haloalkyl, such as —CF₃ or —CHF₂, or a pharmaceutically acceptable salt thereof.

108. The compound according to any one of the preceding claims, or a pharmaceutically acceptable salt thereof, wherein R³ is selected from the group consisting of:

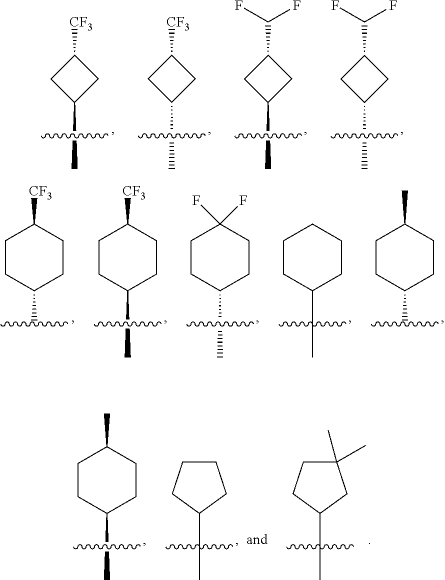

109. The compound according to any one of the preceding claims, wherein R³ is C₁₋₃ haloalkyl, such as —(CH₂)₂CF₃, or a pharmaceutically acceptable salt thereof.

110. The compound according to any one of the preceding claims, wherein R³ is C₅₋₈ spiroalkyl optionally substituted with 1 to 4 substituents independently selected from the group consisting of halogen, C₁₋₃ alkyl and C₁₋₃ haloalkyl, or a pharmaceutically acceptable salt thereof.

111. The compound according to any one of the preceding claims, or a pharmaceutically acceptable salt thereof, wherein R³ is

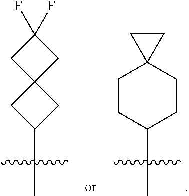 [SMILES: *C1CC2(C1)CC(F)(F)C2 ; *C1CCC2(CC1)CC2]

112. The compound according to any one of the preceding claims, wherein R³ is C₅₋₈ tricycloalkyl optionally substituted with 1 to 4 substituents independently selected from the group consisting of halogen, C₁₋₃ alkyl and C₁₋₃ haloalkyl, or a pharmaceutically acceptable salt thereof.

113. The compound according to any one of the preceding claims, wherein R³ is C₅₋₈ bicycloalkyl optionally substituted with 1 to 4 substituents independently selected from the group consisting of halogen, C₁₋₃ alkyl and C₁₋₃ haloalkyl, or a pharmaceutically acceptable salt thereof.

114. The compound according to any one of the preceding claims, wherein R³ is bicyclo[1.1.1]pentyl optionally substituted with CF₃ or C₁ alkyl, or a pharmaceutically acceptable salt thereof.

115. The compound according to any one of the preceding claims, wherein R³ is 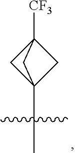 [SMILES: *C12CC(C(F)(F)F)(C1)C2]
or a pharmaceutically acceptable salt thereof.

116. The compound according to any one of the preceding claims, wherein R³ is of Formula VI: 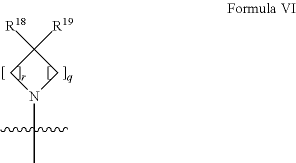
wherein
R¹⁸ is H, halogen, C₁₋₃ alkyl and C₁₋₃ haloalkyl;
R¹⁹ is H, halogen, C₁₋₃ alkyl and C₁₋₃ haloalkyl;
q is 1, 2 or 3; and
r is 1, 2 or 3,
or a pharmaceutically acceptable salt thereof.

117. The compound according to any one of the preceding claims, wherein q is 1, or a pharmaceutically acceptable salt thereof.

118. The compound according to any one of the preceding claims, wherein q is 2, or a pharmaceutically acceptable salt thereof.

119. The compound according to any one of the preceding claims, wherein r is 1, or a pharmaceutically acceptable salt thereof.

120. The compound according to any one of the preceding claims, wherein r is 2, or a pharmaceutically acceptable salt thereof.

121. The compound according to any one of the preceding claims, wherein R¹⁸ is H, or a pharmaceutically acceptable salt thereof.

122. The compound according to any one of the preceding claims, wherein R¹⁸ is halogen, such as F, or a pharmaceutically acceptable salt thereof.

123. The compound according to any one of the preceding claims, wherein R¹⁸ is C₁₋₃ alkyl, such as —CH₃, or a pharmaceutically acceptable salt thereof.

124. The compound according to any one of the preceding claims, wherein R¹⁸ is C₁₋₃ haloalkyl, such as —CF₃ or —CHF₂, or a pharmaceutically acceptable salt thereof.

125. The compound according to any one of the preceding claims, wherein R¹⁹ is H, or a pharmaceutically acceptable salt thereof.

126. The compound according to any one of the preceding claims, wherein R¹⁹ is halogen, such as F, or a pharmaceutically acceptable salt thereof.

127. The compound according to any one of the preceding claims, wherein R¹⁹ is C₁₋₃ alkyl, such as —CH₃, or a pharmaceutically acceptable salt thereof.

128. The compound according to any one of the preceding claims, wherein R¹⁹ is C₁₋₃ haloalkyl, such as —CF₃ or —CHF₂, or a pharmaceutically acceptable salt thereof.

129. The compound according to any one of the preceding claims, or a pharmaceutically acceptable salt thereof, wherein R³ is selected from the group consisting of:

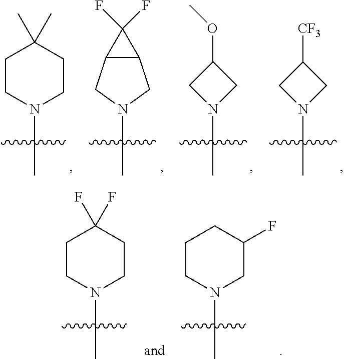

130. The compound according to any one of the preceding claims, wherein R³ is —OCH₂—(C₃₋₆ cycloalkyl) optionally substituted with 1 to 4 substituents independently selected from the group consisting of halogen, C₁₋₃ alkyl, C₁₋₃ haloalkyl, C₁₋₃ alkoxy and C₁₋₃ haloalkoxy, or a pharmaceutically acceptable salt thereof.

131. The compound according to any one of the preceding claims, wherein R³ is 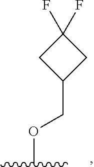 [SMILES: *OCC1CC(F)(F)C1]
or a pharmaceutically acceptable salt thereof.

132. The compound according to any one of the preceding claims, wherein R³ is C₃₋₆ cycloalkyl wherein one or more methylene group is replaced with —O—, or a pharmaceutically acceptable salt thereof.

133. The compound according to any one of the preceding claims, wherein R³ is 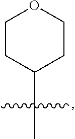 [SMILES: *C1CCOCC1]
or a pharmaceutically acceptable salt thereof.

134. The compound according to any one of the preceding claims, wherein R³ is C₃₋₆ cycloalkyl wherein one or more methylene group is replaced with —N(R²⁸)—, wherein R²⁸ is selected from the group consisting of H, C₁₋₃ alkyl and —C(O)C₁₋₃ alkyl, or a pharmaceutically acceptable salt thereof.

135. The compound according to any one of the preceding claims, wherein R³ is of Formula XV: 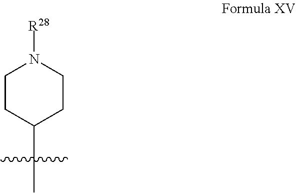
wherein R²⁸ is selected from the group consisting of H, C₁₋₃ alkyl and —C(O)C₁₋₃ alkyl, or a pharmaceutically acceptable salt thereof.

136. The compound according to any one of the preceding claims, or a pharmaceutically acceptable salt thereof, wherein R³ is selected from the group consisting of:

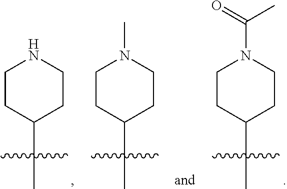

137. The compound according to any one of the preceding claims, wherein R²³ is of Formula XLVII: 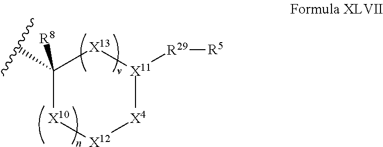
or a pharmaceutically acceptable salt thereof.

138. The compound according to any one of the preceding claims, wherein R²³ is of Formula XLVIII: 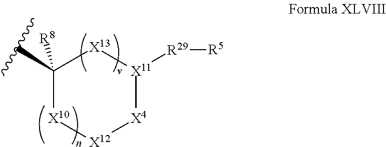
or a pharmaceutically acceptable salt thereof.

139. The compound according to any one of the preceding claims, wherein R²³ is of Formula XLIX: 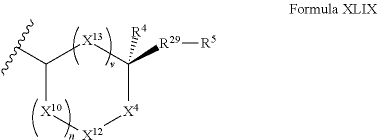
or a pharmaceutically acceptable salt thereof.

140. The compound according to any one of the preceding claims, wherein R²³ is of Formula L: 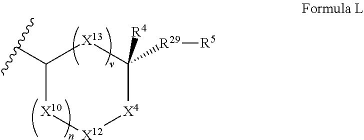
or a pharmaceutically acceptable salt thereof.

141. The compound according to any one of the preceding claims, wherein R²³ is of Formula LI: 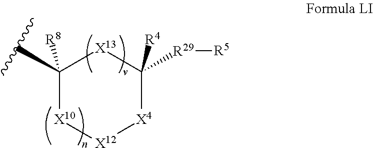
or a pharmaceutically acceptable salt thereof.

142. The compound according to any one of the preceding claims, wherein R²³ is of Formula LII: 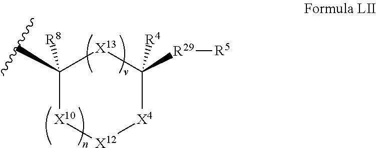
or a pharmaceutically acceptable salt thereof.

143. The compound according to any one of the preceding claims, wherein R²³ is of Formula LIII: 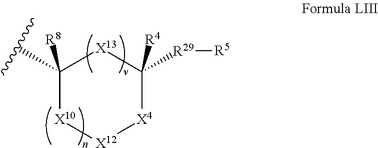
or a pharmaceutically acceptable salt thereof.

144. The compound according to any one of the preceding claims, wherein R²³ is of Formula XLVI, wherein X³ is C(R⁸); R⁸ is a bond; X¹³ is C(R⁴⁰)(R⁴¹); R⁴⁰ is C₁ alkyl; R⁸ and R⁴⁰ are linked together to form a 3-membered ring; R⁴¹ is H; and v is 1

145. The compound according to any one of the preceding claims, wherein R²³ is of Formula LIV: 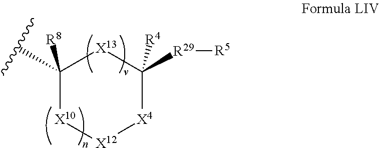
or a pharmaceutically acceptable salt thereof.

146. The compound according to any one of the preceding claims, wherein R²³ is of Formula LV: 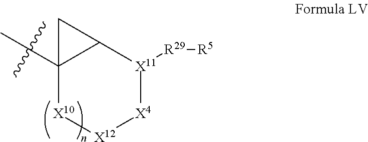
or a pharmaceutically acceptable salt thereof.

147. The compound according to any one of the preceding claims, wherein R²³ is of Formula LVI: 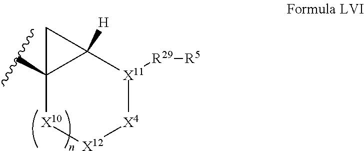
or a pharmaceutically acceptable salt thereof.

148. The compound according to any one of the preceding claims, wherein R²³ is of Formula LVII: 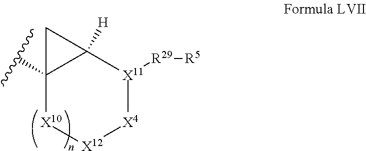
or a pharmaceutically acceptable salt thereof.

149. The compound according to any one of the preceding claims, wherein R²³ is of Formula LVIII: 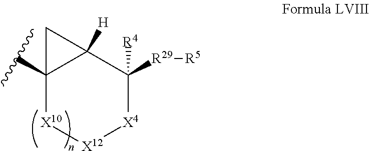
or a pharmaceutically acceptable salt thereof.

150. The compound according to any one of the preceding claims, wherein R²³ is of Formula LIX: 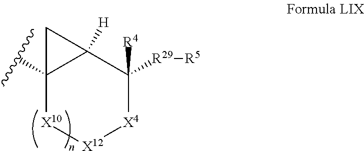
or a pharmaceutically acceptable salt thereof.

151. The compound according to any one of the preceding claims, wherein R²³ is a 6 membered ring, or a pharmaceutically acceptable salt thereof.

152. The compound according to any one of the preceding claims, wherein X³ is N; X⁴ is O; X¹⁰ is C(R³⁷)(R⁷); X¹¹ is C(R⁴); X¹² is C(R⁶)(R^{6b}); X¹³ is C(R⁴⁰)(R⁴¹);
n is 1; and v is 1, or a pharmaceutically acceptable salt thereof.

153. The compound according to any one of the preceding claims, wherein X³ is N; X⁴ is NH or NR⁹; X¹⁰ is C(R³⁷)(R⁷); X¹¹ is C(R⁴); X¹² is C(R¹)(R^{6b}); X¹³ is C(R⁴⁰)(R⁴¹); n is 1; and v is 1, or a pharmaceutically acceptable salt thereof.

154. The compound according to any one of the preceding claims, wherein X³ is N; X⁴ is C(R³⁸)(R³⁹); X¹¹ is C(R³⁷)(R⁷); X¹¹ is C(R⁴); X¹² is C(R⁶)(R^{6b}); X¹³ is C(R⁴⁰)(R⁴¹); n is 1; and v is 1, or a pharmaceutically acceptable salt thereof.

155. The compound according to any one of the preceding claims, wherein X³ is N; X⁴ is a bond; X¹⁰ is C(R³⁷)(R⁷); X¹¹ is C(R⁴); X¹² is C(R⁶)(R^{6b}); X¹³ is C(R⁴⁰)(R⁴¹); n is 1; and v is 2, or a pharmaceutically acceptable salt thereof.

156. The compound according to any one of the preceding claims, wherein X³ is C(R⁸); X⁴ is O; X¹⁰ is C(R³⁷)(R⁷); X¹¹ is C(R⁴); X¹² is C(R⁶)(R^{6b}); X¹³ is C(R⁴⁰)(R⁴¹); n is 1; and v is 1, or a pharmaceutically acceptable salt thereof

157. The compound according to any one of the preceding claims, wherein X³ is N, or a pharmaceutically acceptable salt thereof.

158. The compound according to any one of the preceding claims, wherein X³ is C(R⁸), or a pharmaceutically acceptable salt thereof.

159. The compound according to any one of the preceding claims, wherein R⁸ is H, or a pharmaceutically acceptable salt thereof.

160. The compound according to any one of the preceding claims, wherein X⁴ is O, or a pharmaceutically acceptable salt thereof.

161. The compound according to any one of the preceding claims, wherein X⁴ is CF₂, or a pharmaceutically acceptable salt thereof.

162. The compound according to any one of the preceding claims, wherein X⁴ is CH₂, or a pharmaceutically acceptable salt thereof.

163. The compound according to any one of the preceding claims, wherein X⁴ is CHF, or a pharmaceutically acceptable salt thereof.

164. The compound according to any one of the preceding claims, wherein R³⁸ is H, or a pharmaceutically acceptable salt thereof.

165. The compound according to any one of the preceding claims, wherein R³⁸ is F, or a pharmaceutically acceptable salt thereof.

166. The compound according to any one of the preceding claims, wherein R³⁹ is F, or a pharmaceutically acceptable salt thereof.

167. The compound according to any one of the preceding claims, wherein R³⁹ is F, or a pharmaceutically acceptable salt thereof.

168. The compound according to any one of the preceding claims, wherein R³⁸ is H and R³⁹ is H, or a pharmaceutically acceptable salt thereof.

169. The compound according to any one of the preceding claims, wherein R³⁸ is F and R³⁹ is F, or a pharmaceutically acceptable salt thereof.

170. The compound according to any one of the preceding claims, wherein R³⁸ is H and R³⁹ is F, or a pharmaceutically acceptable salt thereof.

171. The compound according to any one of the preceding claims, wherein R⁴ is H, or a pharmaceutically acceptable salt thereof.

172. The compound according to any one of the preceding claims, wherein R⁴ and R⁵ are linked together to form a ring, or a pharmaceutically acceptable salt thereof.

173. The compound according to any one of the preceding claims, wherein R²³ is of Formula XLVI, wherein X³ is C(R⁸); R⁸ is a bond; X¹³ is C(R⁴⁰)(R⁴¹); R⁴⁰ is C₁ alkyl; R⁸ and R⁴⁰ are linked together to form a 3-membered ring; R⁴¹ is H; v is 1; X¹¹ is C(H), X⁴ is O; X¹² is CH₂; X¹⁰ is CH₂; and n is 1; or a pharmaceutically acceptable salt thereof.

174. The compound according to any one of the preceding claims, wherein R²³ is of Formula LX: 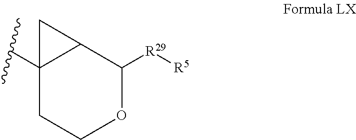
or a pharmaceutically acceptable salt thereof.

175. The compound according to any one of the preceding claims, wherein X¹² is C(R⁶)(R^{6b}); R^{6b} is H; R⁶ is C₁₋₃ alkyl; n is 1; X¹⁰ is C(R³⁷)(R⁷); R³⁷ is H; and R⁷ is a bond to R⁶, or a pharmaceutically acceptable salt thereof.

176. The compound according to any one of the preceding claims, wherein R²³ is of Formula XLVI, wherein X³ is N; X¹³ is CH₂; v is 1; X¹¹ is C(H); X⁴ is O; X¹² is C(R⁶)(R^{6b}); R^{6b} is H; R⁶ is C₁ alkyl; n is 1; X¹⁰ is C(R³⁷)(R⁷); R³⁷ is H; and R⁷ is a bond to R⁶, or a pharmaceutically acceptable salt thereof.

177. The compound according to any one of the preceding claims, wherein R²³ is of Formula LXIII: 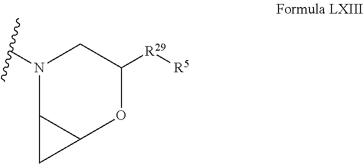
or a pharmaceutically acceptable salt thereof.

178. The compound according to any one of the preceding claims, wherein R²³ is of Formula XVI, wherein X³ is N; X¹⁰ is CH₂; n is 1; v is 1; R⁶ is H; X⁴ is O; and; X¹¹ is C(H), or a pharmaceutically acceptable salt thereof.

179. The compound according to any one of the preceding claims, wherein R²³ is of Formula XX, wherein X³ is N; X¹⁰ is CH₂; n is 1; v is 1; X⁴ is O; and; X¹¹ is C(H), or a pharmaceutically acceptable salt thereof.

180. The compound according to any one of the preceding claims, wherein R²³ is of Formula XXXIV: 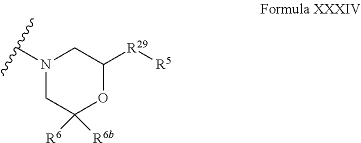
or a pharmaceutically acceptable salt thereof.

181. The compound according to any one of the preceding claims, wherein R²³ is of Formula XXXIV, R⁶ is H and R^{6b} is H, or a pharmaceutically acceptable salt thereof.

182. The compound according to any one of the preceding claims, wherein R²³ is of Formula XXXIV, R⁶ is F and R^{6b} is F, or a pharmaceutically acceptable salt thereof.

183. The compound according to any one of the preceding claims, wherein R²³ is of Formula XXXIV, R⁶ is CH₃ and R^{6b} is CH₃, or a pharmaceutically acceptable salt thereof.

184. The compound according to any one of the preceding claims, wherein R²³ is of Formula XXXIV, R⁶ is CH₃ and R^{6b} is H, or a pharmaceutically acceptable salt thereof.

185. The compound according to any one of the preceding claims, wherein R²³ is of Formula XLV: 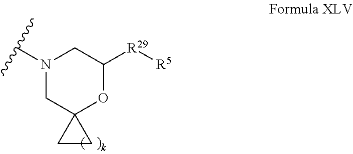
wherein k is 1, 2, 3 or 4, or a pharmaceutically acceptable salt thereof.

186. The compound according to any one of the preceding claims, wherein k is 1, or a pharmaceutically acceptable salt thereof.

187. The compound according to any one of the preceding claims, wherein k is 2, or a pharmaceutically acceptable salt thereof.

188. The compound according to any one of the preceding claims, wherein R²³ is of Formula XVI, wherein X³ is N; X¹⁰ is CH₂; n is 1; v is 1; R⁶ is H; X⁴ is C(R³³)(R³⁴); R³³ is H or F; R³⁴ is H or F; and; X¹¹ is C(H), or a pharmaceutically acceptable salt thereof.

189. The compound according to any one of the preceding claims, wherein R²³ is of Formula XXXV: 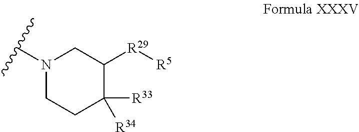
wherein R³³ is H or F, and R³⁴ is H or F, or a pharmaceutically acceptable salt thereof.

190. The compound according to any one of the preceding claims, wherein R²³ is of Formula XVI, wherein X³ is N; n is 0; v is 1; R⁶ is H; X⁴ is CH₂; and; X¹¹ is C(H), or a pharmaceutically acceptable salt thereof.

191. The compound according to any one of the preceding claims, wherein R²³ is of Formula XXXVI: 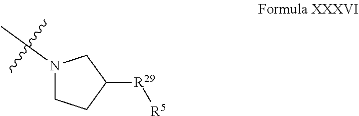
or a pharmaceutically acceptable salt thereof.

192. The compound according to any one of the preceding claims, wherein R²³ is of Formula XVI, wherein X³ is N; X¹⁰ is CH₂; n is 1; v is 1; R⁶ is H; X⁴ is N(R⁹); and; X¹¹ is C(H), or a pharmaceutically acceptable salt thereof.

193. The compound according to any one of the preceding claims, wherein R²³ is of Formula XXXVII: 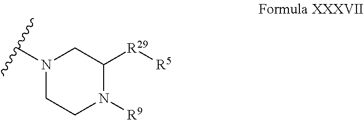
or a pharmaceutically acceptable salt thereof.

194. The compound according to any one of the preceding claims, wherein R²³ is of Formula XVI, wherein X³ is C(H); X¹⁰ is CH₂; n is 1; v is 1; R⁶ is H; X⁴ is O; and; X¹¹ is C(H), or a pharmaceutically acceptable salt thereof.

195. The compound according to any one of the preceding claims, wherein R²³ is of Formula XXXVIII: 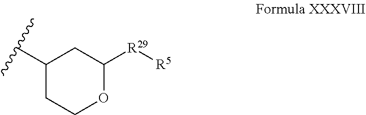
or a pharmaceutically acceptable salt thereof.

196. The compound according to any one of the preceding claims, wherein R²³ is of Formula XVI, wherein X³ is C(H); X¹⁰ is CH₂; n is 1; v is 1; R⁶ is H; X⁴ is C(R³³)(R³⁴); R³³ is H or F; R³⁴ is H or F; and X¹¹ is N, or a pharmaceutically acceptable salt thereof.

197. The compound according to any one of the preceding claims, wherein R²³ is of Formula XXXIX: 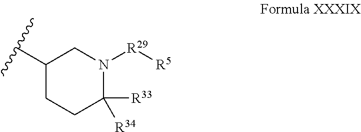
wherein R³³ is H or F, and R³⁴ is H or F, or a pharmaceutically acceptable salt thereof.

198. The compound according to any one of the preceding claims, wherein R³³ is H and R³⁴ is H, or a pharmaceutically acceptable salt thereof.

199. The compound according to any one of the preceding claims, wherein R³³ is F and R³⁴ is F, or a pharmaceutically acceptable salt thereof.

200. The compound according to any one of the preceding claims, wherein R²³ is of Formula XVI, wherein X³ is N; n is 0; v is 1; R⁶ is H; X⁴ is a bond; and X¹¹ is C(H), or a pharmaceutically acceptable salt thereof.

201. The compound according to any one of the preceding claims, wherein R²³ is of Formula XL: 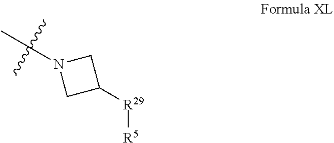
or a pharmaceutically acceptable salt thereof.

202. The compound according to any one of the preceding claims, wherein R²³ is of Formula XVI, wherein X³ is N; X¹⁰ is CH₂; n is 1; v is 2; R⁶ is H; X⁴ is a bond; and X¹¹ is C(H), or a pharmaceutically acceptable salt thereof.

203. The compound according to any one of the preceding claims, wherein R²³ is of Formula XLI: 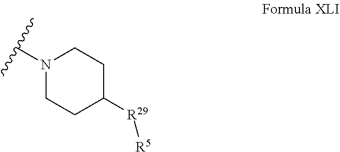
or a pharmaceutically acceptable salt thereof.

204. The compound according to any one of the preceding claims, wherein R²³ is a 5 membered ring, or a pharmaceutically acceptable salt thereof.

205. The compound according to any one of the preceding claims, wherein n is 1, v is 1 and X⁴ is a bond; or n is 0, v is 1 and X⁴ is not a bond, or a pharmaceutically acceptable salt thereof.

206. The compound according to any one of the preceding claims, wherein X³ is N; X¹³ is C(R⁴⁰)(R⁴¹); X¹¹ is C(R⁴); X⁴ is C(R³⁸)(R³⁹); X¹² is C(R⁶)(R^{6b}); and n is O, or a pharmaceutically acceptable salt thereof.

207. The compound according to any one of the preceding claims, wherein R²³ is a 4 membered ring, or a pharmaceutically acceptable salt thereof.

208. The compound according to any one of the preceding claims, wherein n is 0, v is 1 and X⁴ is a bond, n is 1, v is 0 and X⁴ is a bond, or a pharmaceutically acceptable salt thereof.

209. The compound according to any one of the preceding claims, wherein X³ is N; X⁴ is a bond; n is 0; X¹¹ is C(R⁴); X¹² is C(R⁶)(R^{6b}); X¹³ is C(R⁴⁰)(R⁴¹); and v is 1, or a pharmaceutically acceptable salt thereof. The compound according to any one of the preceding claims, wherein R⁵ is selected from the group consisting of C₁₋₆ alkyl, C₁₋₆ haloalkyl, diC₁₋₃ alkylamino, —C(O)—O—(C₁₋₆ alkyl), C₃₋₆ cycloalkyl, C₃₋₆ heterocycloalkyl, phenyl, —O-phenyl, 5-membered heteroaryl, —O-(5-membered heteroaryl), 6-membered heteroaryl and —O-(6-membered heteroaryl), wherein
(a) the C₃₋₆ cycloalkyl or C₃₋₆ heterocycloalkyl is optionally substituted with 1 to 3 substituents independently selected from oxo (═O), C₁₋₃ alkoxy and halogen;
(b) the phenyl, —O-phenyl, 5-membered heteroaryl, —O-(5-membered heteroaryl), 6-membered heteroaryl or —O-(6-membered heteroaryl) is optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl of subsection (b) are optionally substituted with —OH, and
wherein the C₃₋₆ heterocycloalkyl of subsection (b) is optionally substituted with 1 to 3 substituents individually selected from the group consisting of halogen, C₁₋₃ alkyl and —C(O)O(C₁₋₆ alkyl);
(c) the C₁₋₆ alkyl is optionally substituted with 1 to 3 substituents independently selected from —N(H)C(O)R²⁴, -oxo (═O) and —N(R³⁰)(R³¹);
or a pharmaceutically acceptable salt thereof.

210. The compound according to any one of the preceding claims, wherein R⁵ is a 5-membered heteroaryl optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with 1 to 3 substituents independently selected from the group consisting of C₁₋₆ alkyl, C₁₋₆ alkoxy, halogen and —OH,
and wherein the C₃₋₆ heterocycloalkyl is optionally substituted with 1 to 3 substituents individually selected from the group consisting of halogen, C₁₋₃ alkyl and —C(O)O(C₁₋₆ alkyl),
or a pharmaceutically acceptable salt thereof.

211. The compound according to any one of the preceding claims, wherein R⁵ is a 5-membered heteroaryl optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and wherein the C₃₋₆ heterocycloalkyl is optionally substituted with 1 to 3 substituents individually selected from the group consisting of halogen, C₁₋₃ alkyl and —C(O)O(C₁₋₆ alkyl),
or a pharmaceutically acceptable salt thereof.

212. The compound according to any one of the preceding claims, wherein R⁵ is of Formula XXI: 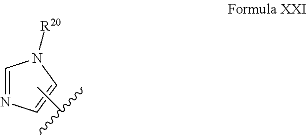
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

213. The compound according to any one of the preceding claims, wherein R⁵ is of Formula XXII: 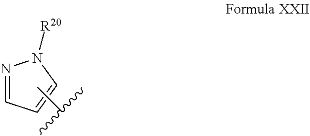
wherein
R²⁰ is C₁₋₃ alkyl, C₃₋₆ cycloalkyl, H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl C₁₋₆ alkoxy, halogen and —OH, or a pharmaceutically acceptable salt thereof.

214. The compound according to any one of the preceding claims, wherein R⁵ is of Formula XXII: 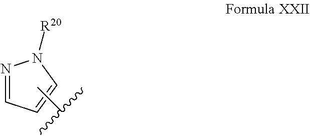
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

215. The compound according to any one of the preceding claims, wherein R⁵ is of Formula XXIII: 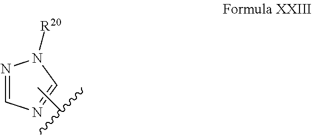
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy, or a pharmaceutically acceptable salt thereof.

216. The compound according to any one of the preceding claims, wherein R⁵ is of Formula XXIV: 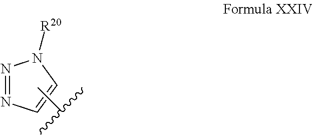
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

217. The compound according to any one of the preceding claims, R⁵ is of Formula XLIII: 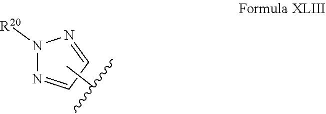
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

218. The compound according to any one of the preceding claims, R⁵ is 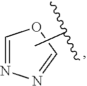
or a pharmaceutically acceptable salt thereof.

219. The compound according to any one of the preceding claims, wherein R⁵ is 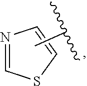
or a pharmaceutically acceptable salt thereof.

220. The compound according to any one of the preceding claims, wherein R⁵ is of Formula VII: 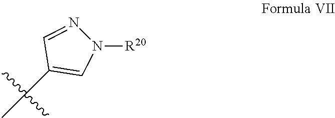
wherein
R²⁰ is C₁₋₃ alkyl, C₃₋₆ cycloalkyl, H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl C₁₋₆ alkoxy, halogen and —OH, or a pharmaceutically acceptable salt thereof.

221. The compound according to any one of the preceding claims, wherein R⁵ is of Formula VII: 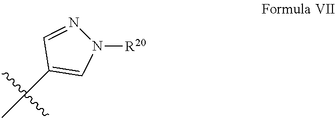
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy, or a pharmaceutically acceptable salt thereof.

222. The compound according to any one of the preceding claims, wherein R⁵ is of Formula XXV: 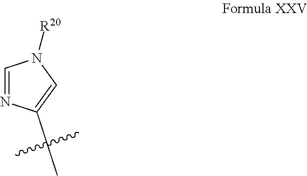
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy, or a pharmaceutically acceptable salt thereof.

223. The compound according to any one of the preceding claims, wherein R⁵ is of Formula XXVI: 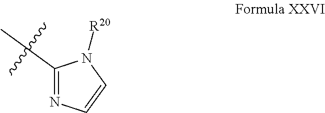
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

224. The compound according to any one of the preceding claims, wherein R⁵ is of Formula XXVII: 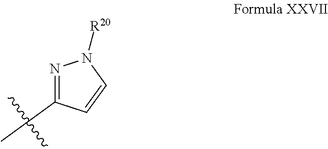
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

225. The compound according to any one of the preceding claims, wherein R⁵ is of Formula XXVIII: 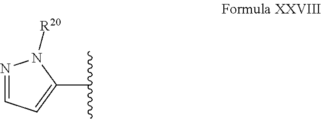
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₆ alkyl and C₁₋₆ alkoxy, or a pharmaceutically acceptable salt thereof.

226. The compound according to any one of the preceding claims, wherein R⁵ is of Formula XXIX: 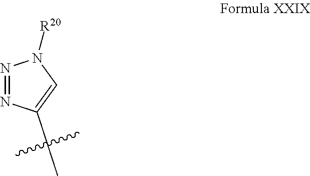
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

227. The compound according to any one of the preceding claims, wherein R⁵ is of Formula XXX: 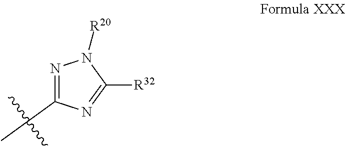
wherein
R³² is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy and
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

228. The compound according to any one of the preceding claims, wherein R⁵ is of Formula XLII: 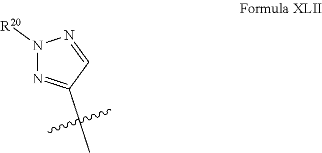
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

229. The compound according to any one of the preceding claims, wherein R⁵ is of Formula XXXI: 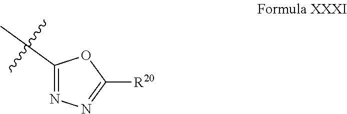
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

230. The compound according to any one of the preceding claims, wherein R⁵ is of Formula XXXII: 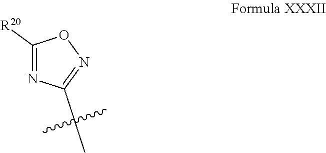
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

231. The compound according to any one of the preceding claims, wherein R⁵ is of Formula XXXIII: 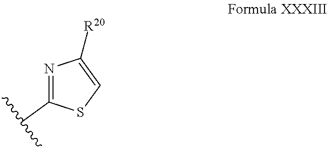
wherein
R²⁰ is C₁₋₃ alkyl, C₃₋₆ cycloalkyl H or C₃₋₆ heterocycloalkyl, wherein the C₁₋₃ alkyl, C₃₋₆ cycloalkyl or C₃₋₆ heterocycloalkyl is optionally substituted with 1 to 3 substituents independently selected from the group consisting of C₁₋₅ alkyl and C₁₋₆ alkoxy, or a pharmaceutically acceptable salt thereof.

232. The compound according to any one of the preceding claims, wherein R²⁰ is C₁₋₃ alkyl, such as —CH₃, or a pharmaceutically acceptable salt thereof.

233. The compound according to any one of the preceding claims, wherein R²⁰ is C₁₋₃ alkyl optionally substituted with C₁₋₆ alkoxy, or a pharmaceutically acceptable salt thereof.

234. The compound according to any one of the preceding claims, wherein R²⁰ is C₁₋₂ alkyl optionally substituted with C, alkoxy, or a pharmaceutically acceptable salt thereof.

235. The compound according to any one of the preceding claims, wherein R²⁰ is C₁₋₃ alkyl optionally substituted with 1 to 3 halogens, such as F, or a pharmaceutically acceptable salt thereof.

236. The compound according to any one of the preceding claims, wherein R²⁰ is methyl optionally substituted with 1 to 3 halogens, such as F, or a pharmaceutically acceptable salt thereof.

237. The compound according to any one of the preceding claims, wherein R²⁰ is —CF₃, or a pharmaceutically acceptable salt thereof.

238. The compound according to any one of the preceding claims, wherein R²⁰ is —CH₂CH₂OCH₃, or a pharmaceutically acceptable salt thereof.

239. The compound according to any one of the preceding claims, wherein R²⁰ C₃₋₆ cycloalkyl, such as C₃ cycloalkyl, or a pharmaceutically acceptable salt thereof.

240. The compound according to any one of the preceding claims, wherein R²⁰ is H, or a pharmaceutically acceptable salt thereof.

241. The compound according to any one of the preceding claims, wherein R⁵ is selected from the group consisting of: 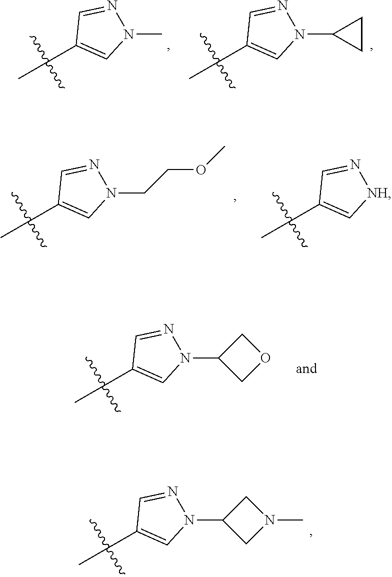
or a pharmaceutically acceptable salt thereof.

242. The compound according to any one of the preceding claims, wherein R⁵ 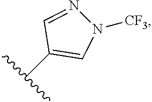 [SMILES: *c1cnn(C(F)(F)F)c1]
or a pharmaceutically acceptable salt thereof.

243. The compound according to any one of the preceding claims, wherein R⁵ is 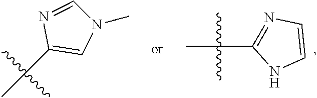
or a pharmaceutically acceptable salt thereof.

244. The compound according to any one of the preceding claims, wherein R⁵ is selected from the group consisting of 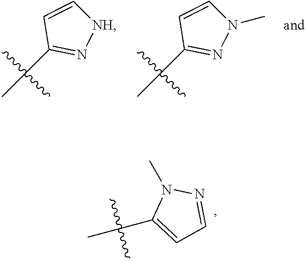
or a pharmaceutically acceptable salt thereof.

245. The compound according to any one of the preceding claims, wherein R⁵ is selected from the group consisting of 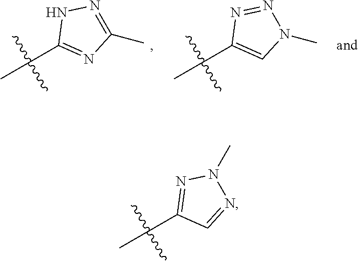
or a pharmaceutically acceptable salt thereof.

246. The compound according to any one of the preceding claims, wherein R⁵ is selected from the group consisting of 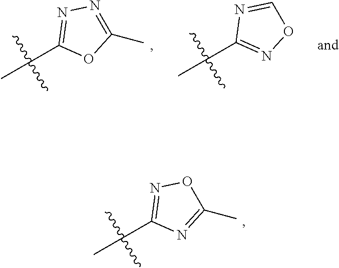
or a pharmaceutically acceptable salt thereof.

247. The compound according to any one of the preceding claims, wherein R⁵ is 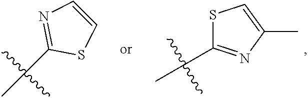
or a pharmaceutically acceptable salt thereof.

248. The compound according to any one of the preceding claims, wherein R⁵ is 6-membered heteroaryl optionally substituted with 1 to 3 substituents independently selected from the group consisting of halogen, C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and wherein the C₃₋₆ heterocycloalkyl is optionally substituted with 1 to 3 substituents individually selected from the group consisting of halogen, C₁₋₃ alkyl and —C(O)O(C₁₋₆ alkyl),
or a pharmaceutically acceptable salt thereof.

249. The compound according to any one of the preceding claims, wherein R⁵ is of Formula XLIV: 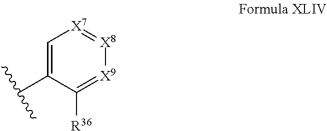
wherein
X⁷ is N or CH;
X³ is N or C(R²¹);
X⁹ is N or C(R²²);
R²¹ is C₁₋₃ alkyl, such as —CH₃;
R²² is C₁₋₃ alkyl, such as —CH₃, and
R³¹ is H or C₁₋₃ alkyl, such as —CH₃,
or a pharmaceutically acceptable salt thereof.

250. The compound according to any one of the preceding claims, wherein R⁵ is of Formula VIII: 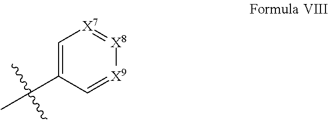
wherein
X⁷ is N or CH;
X⁸ is N or C(R²¹);
X⁹ is N or C(R²²);
R²¹ is C₁₋₃ alkyl, such as —CH₃; and
R²² is C₁₋₃ alkyl, such as —CH₃,
or a pharmaceutically acceptable salt thereof.

251. The compound according to any one of the preceding claims, wherein R⁵ is selected from the group consisting of 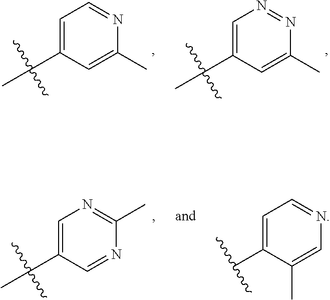
or a pharmaceutically acceptable salt thereof.

252. The compound according to any one of the preceding claims, wherein R⁵ is of Formula XIII: 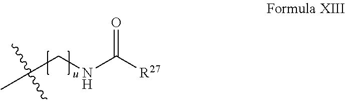
wherein
R²⁷ is C₁₋₆ alkyl; and
u is 1, 2 or 3,
or a pharmaceutically acceptable salt thereof.

253. The compound according to any one of the preceding claims, wherein R⁵ is C₁₋₆ alkyl optionally substituted with 1 to 3 substituents independently selected from —N(H)C(O)R²⁴, -oxo (═O), —O—C₁₋₃ alkyl and —N(R³⁰)(R³¹), or a pharmaceutically acceptable salt thereof.

254. The compound according to any one of the preceding claims, wherein R⁵ is C₁₋₃ alkyl optionally substituted with —O—C₁₋₃ alkyl, such as with —O—C₁ alkyl, or a pharmaceutically acceptable salt thereof, or a pharmaceutically acceptable salt thereof.

255. The compound according to any one of the preceding claims, wherein R⁵ is C₁₋₃ alkyl optionally substituted with —N(R³⁰)(R³¹), wherein R³⁰ and R³¹ are individually selected from H and C₁₋₃ alkyl, such as C₁ alkyl, or a pharmaceutically acceptable salt thereof.

256. The compound according to any one of the preceding claims, wherein R⁵ is selected from the group consisting of: 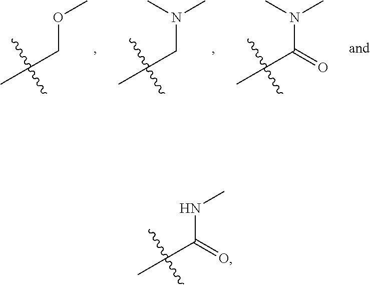
or a pharmaceutically acceptable salt thereof.

257. The compound according to any one of the preceding claims, wherein R⁵ is C₃₋₆ heterocycloalkyl optionally substituted with 1 to 3 substituents independently selected from oxo (═O), C₁₋₃ alkoxy and halogen, or a pharmaceutically acceptable salt thereof.

258. The compound according to any one of the preceding claims, wherein R⁵ is C₄ heterocycloalkyl optionally substituted with 1 to 3 substituents independently selected from C₁₋₃ alkoxy and halogen, or a pharmaceutically acceptable salt thereof.

259. The compound according to any one of the preceding claims, wherein R⁵ is C₃₋₆ heterocycloalkyl optionally substituted with 1 to 3 substituents independently selected from oxo (═O), C₁₋₃ alkoxy and halogen. or a pharmaceutically acceptable salt thereof.

260. The compound according to any one of the preceding claims, wherein R⁵ is C₄ heterocycloalkyl optionally substituted with 1 to 3 substituents independently selected from C₁₋₃ alkoxy and halogen, or a pharmaceutically acceptable salt thereof.

261. The compound according to any one of the preceding claims, wherein R⁵ is an azetidine optionally substituted with 1 to 3 substituents independently selected from C₁₋₃ alkoxy and halogen, or a pharmaceutically acceptable salt thereof.

262. The compound according to any one of the preceding claims, wherein R⁵ is 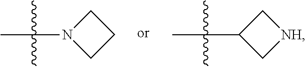 [SMILES: *N1CCC1 ; *C1CNC1]
optionally substituted with 1 or 2 substituents independently selected from C₁₋₃ alkoxy, such as C, alkoxy, and halogen, such as F, or a pharmaceutically acceptable salt thereof.

263. The compound according to any one of the preceding claims, wherein R⁵ is: 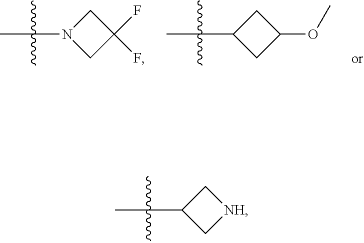
or a pharmaceutically acceptable salt thereof.

264. The compounds according to any one of the preceding claims, wherein R⁵ is C heterocycloalkyl optionally substituted with 1 to 3 substituents independently selected from oxo (═O), C₁₋₃ alkoxy and halogen, or a pharmaceutically acceptable salt thereof.

265. The compound according to any one of the preceding claims, wherein R⁵ is: 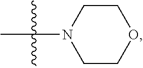 [SMILES: *N1CCOCC1]
or a pharmaceutically acceptable salt thereof.

266. The compound according to any one of the preceding claims, wherein the C₃₋₆ heterocycloalkyl is selected from the group consisting of: 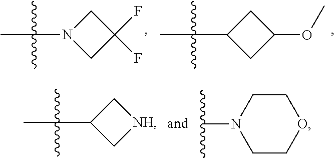
or a pharmaceutically acceptable salt thereof.

267. The compound according to any one of the preceding claims, wherein R⁶ is H, or a pharmaceutically acceptable salt thereof.

268. The compound according to any one of the preceding claims, wherein R⁶ is C₁₋₃ alkyl, such as C₁ alkyl, or a pharmaceutically acceptable salt thereof.

269. The compound according to any one of the preceding claims, wherein R⁶ is halogen.

270. The compound according to any one of the preceding claims, wherein R^{6b} is H, or a pharmaceutically acceptable salt thereof.

271. The compound according to any one of the preceding claims, wherein R^{6b} is C₁₋₃ alkyl, such as C₁ alkyl, or a pharmaceutically acceptable salt thereof.

272. The compound according to any one of the preceding claims, wherein R^{6b} is halogen.

273. The compound according to any one of the preceding claims, wherein R⁶ and R^{6b} are H, or a pharmaceutically acceptable salt thereof.

274. The compound according to any one of the preceding claims, wherein R⁶ and R^{6b} are C₁₋₃ alkyl, such as C₁ alkyl, or a pharmaceutically acceptable salt thereof.

275. The compound according to any one of the preceding claims, wherein R⁶ and R^{6b} are C₁₋₃ alkyl, and R⁶ and R^{6b} are linked together to form a 3 to 6-membered ring, such as a 4-membered ring or a 3-membered ring, or a pharmaceutically acceptable salt thereof.

276. The compound according to any one of the preceding claims, wherein R⁶ and R^{6b} are C₁₋₃ alkyl, and R⁶ and R^{6b} are linked together to form a 3 to 6-membered ring, and one methylene group is replaced with —O—, or a pharmaceutically acceptable salt thereof.

277. The compound according to any one of the preceding claims, wherein R⁶ and R^{6b} are C₁₋₃ alkyl, and R⁶ and R^{6b} are linked together to form a 3 to 6-membered ring, and one methylene group is replaced with —N(R³⁵)—, wherein R³⁵ is selected from the group consisting of H, C₁₋₃ alkyl and —C(O)C₁₋₃ alkyl, or a pharmaceutically acceptable salt thereof.

278. The compound according to any one of the preceding claims, wherein X¹⁰ is C(H)(R⁷), or a pharmaceutically acceptable salt thereof.

279. The compound according to any one of the preceding claims, wherein R⁷ is H, or a pharmaceutically acceptable salt thereof.

280. The compound according to any one of the preceding claims, wherein R³⁷ is H, or a pharmaceutically acceptable salt thereof.

281. The compound according to any one of the preceding claims, wherein R⁷ is H and R³⁷ is H, or a pharmaceutically acceptable salt thereof.

282. The compound according to any one of the preceding claims, wherein X¹⁰ is C(H)₂, or a pharmaceutically acceptable salt thereof.

283. The compound according to any one of the preceding claims, wherein R²³ is selected from the group consisting of: 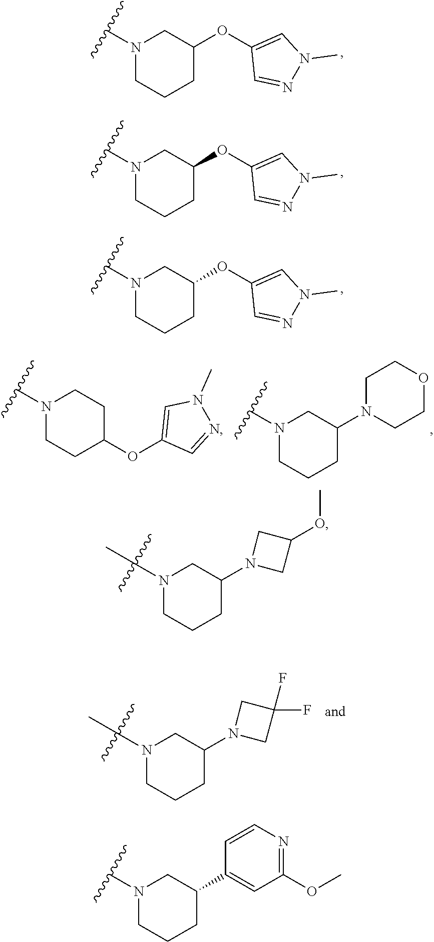
or a pharmaceutically acceptable salt thereof.

284. The compound according to any one of the preceding claims, wherein R²³ is selected from the group consisting of: 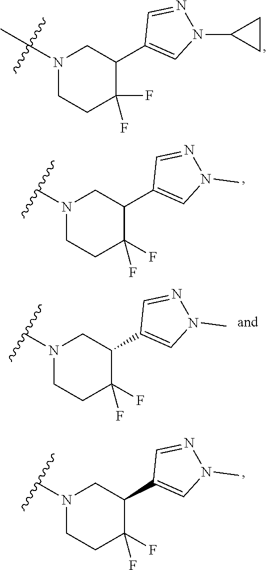
or a pharmaceutically acceptable salt thereof.

285. The compound according to any one of the preceding claims, wherein R²³ is selected from the group consisting of: 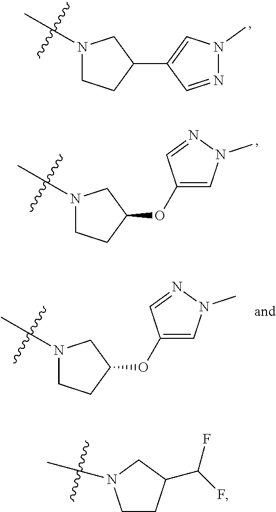
or a pharmaceutically acceptable salt thereof.

286. The compound according to any one of the preceding claims, wherein R²³ is selected from the group consisting of: 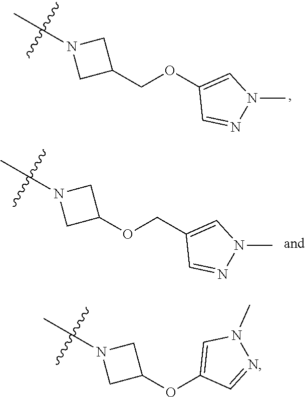
or a pharmaceutically acceptable salt thereof.

287. The compound according to any one of the preceding claims, wherein R²³ is selected from the group consisting of: 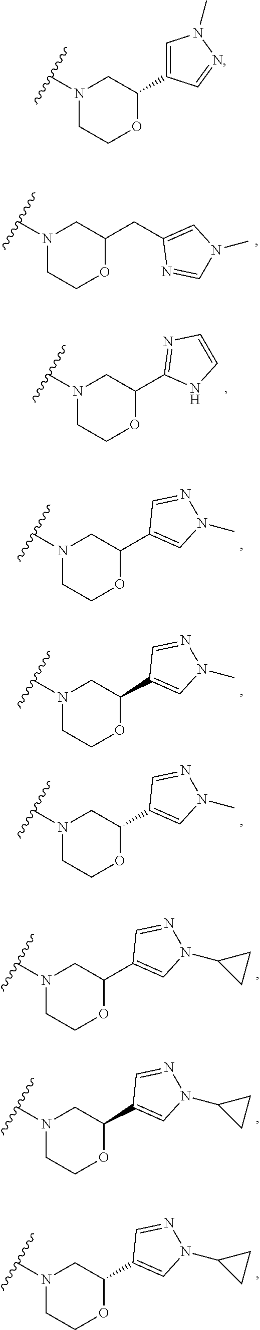 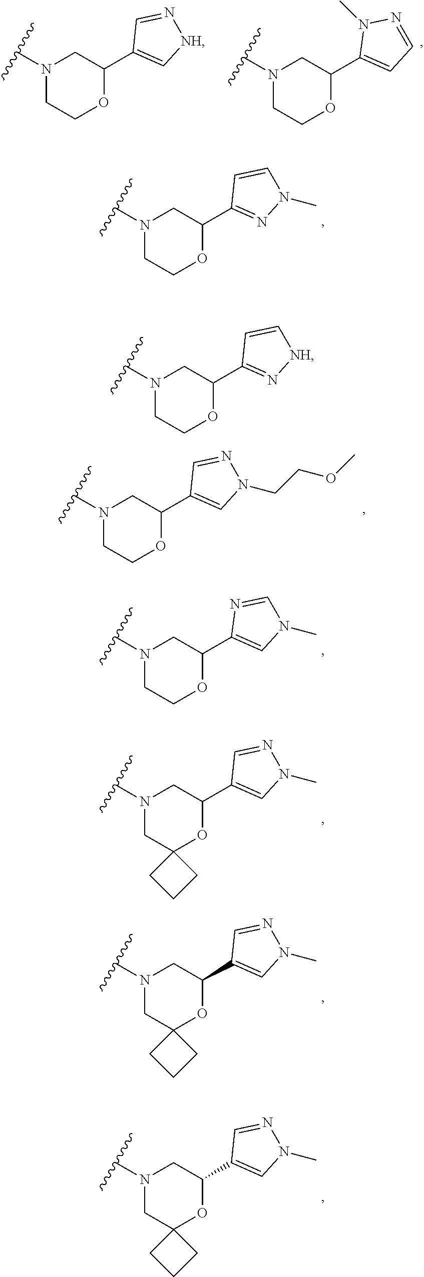 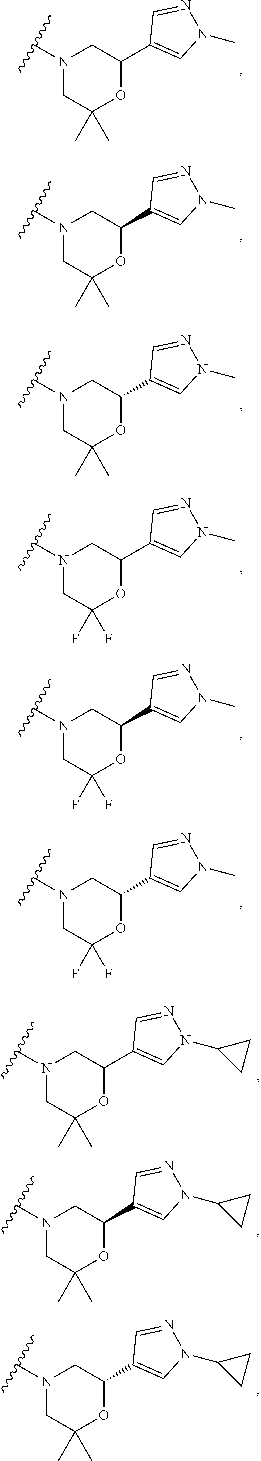 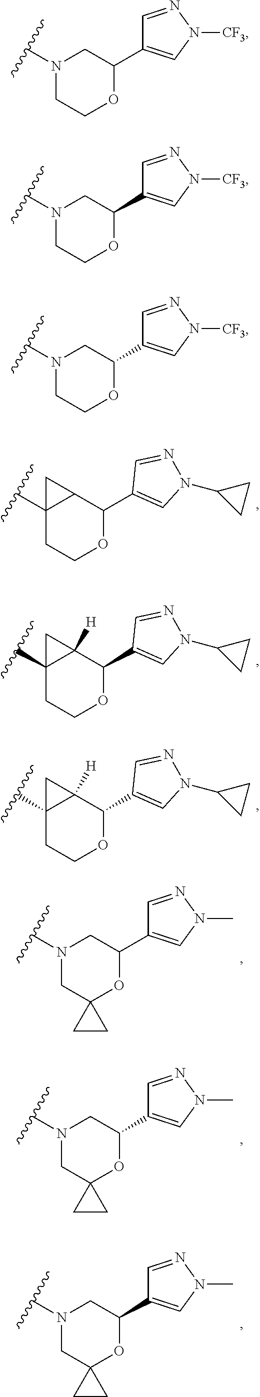 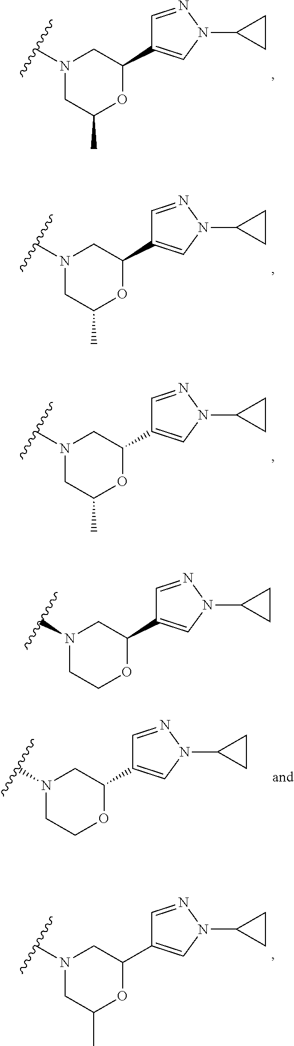
or a pharmaceutically acceptable salt thereof.

288. The compound according to any one of the preceding claims, wherein R²³ is selected from the group consisting of: 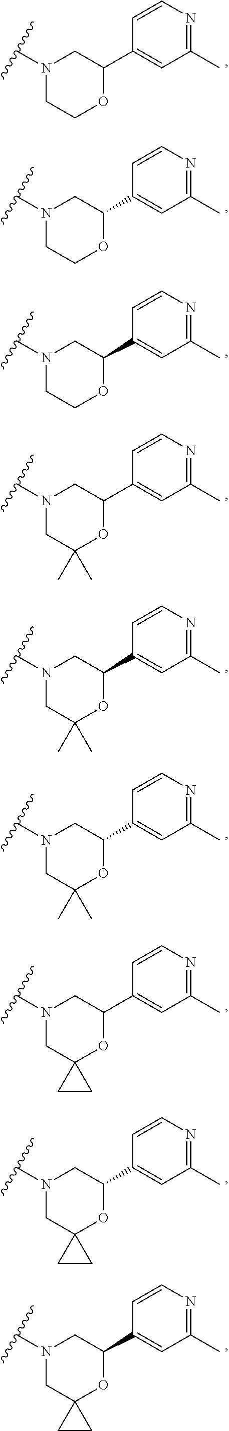 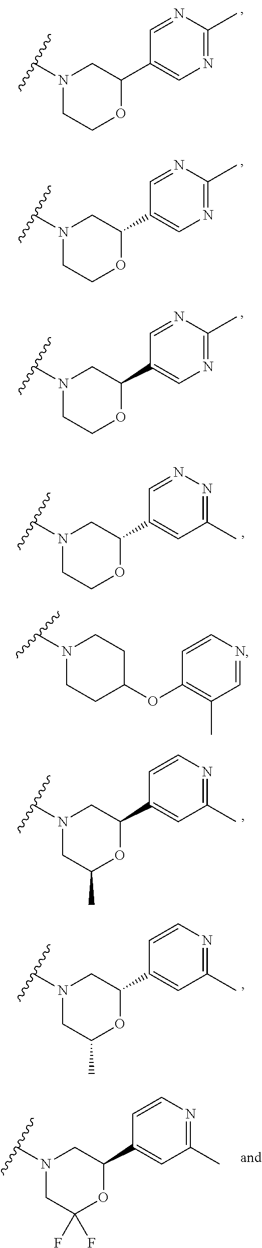 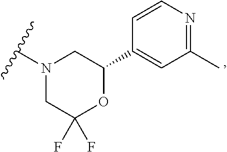 [SMILES: *N1C[C@H](c2ccnc(C)c2)OC(F)(F)C1]
or a pharmaceutically acceptable salt thereof.

289. The compound according to any one of the preceding claims, wherein R²³ is 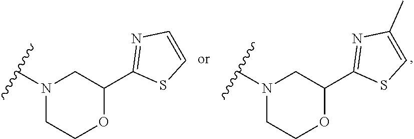 [SMILES: *N1CCOC(c2nccs2)C1 ; *N1CCOC(c2nc(C)cs2)C1]
or a pharmaceutically acceptable salt thereof.

290. The compound according to any one of the preceding claims, wherein R²³ is selected from the group consisting of: 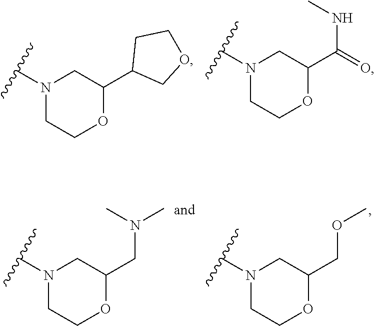
or a pharmaceutically acceptable salt thereof.

291. The compound according to any one of the preceding claims, wherein R²³ is selected from the group consisting of: 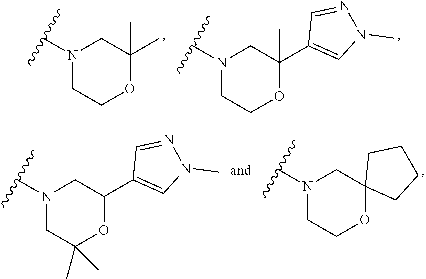
or a pharmaceutically acceptable salt thereof.

292. The compound according to any one of the preceding claims, wherein R²³ is selected from the group consisting of: 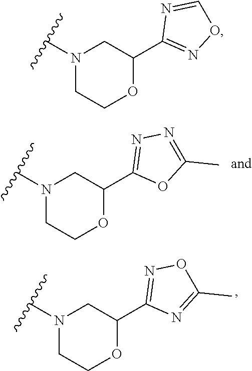
or a pharmaceutically acceptable salt thereof.

293. The compound according to any one of the preceding claims, wherein R²³ is selected from the group consisting of: 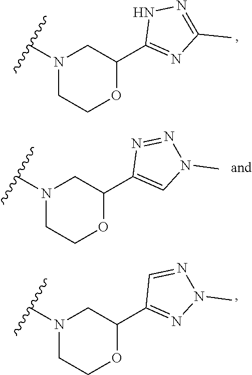 [SMILES: *N1CCOC(c2nc(C)n[nH]2)C1 ; *N1CCOC(c2cn(C)nn2)C1 ; *N1CCOC(c2cnn(C)n2)C1]
or a pharmaceutically acceptable salt thereof.

294. The compound according to any one of the preceding claims, wherein R²³ is selected from the group consisting of: 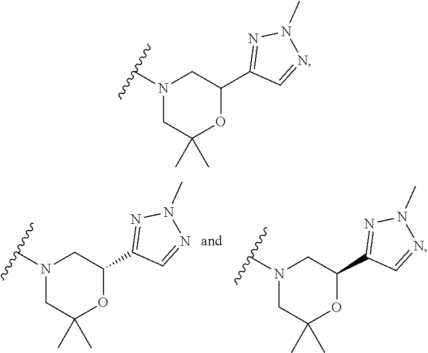
or a pharmaceutically acceptable salt thereof.

295. The compound according to any one of the preceding claims, wherein R²³ is selected from the group consisting of: 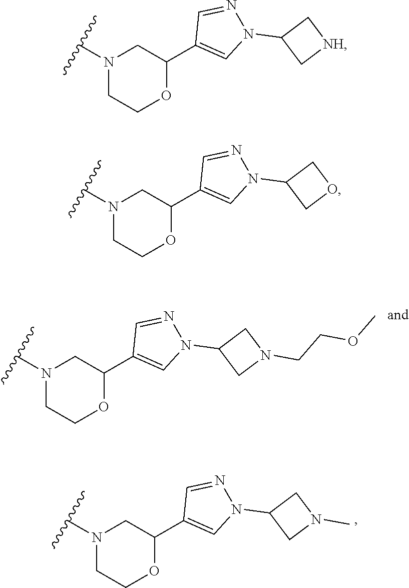 [SMILES: *N1CCOC(c2cnn(C3CNC3)c2)C1 ; *N1CCOC(c2cnn(C3COC3)c2)C1 ; *N1CCOC(c2cnn(C3CN(CCOC)C3)c2)C1 ; *N1CCOC(c2cnn(C3CN(C)C3)c2)C1]
or a pharmaceutically acceptable salt thereof.

296. The compound according to any one of the preceding claims, wherein R²³ is selected from the group consisting of: 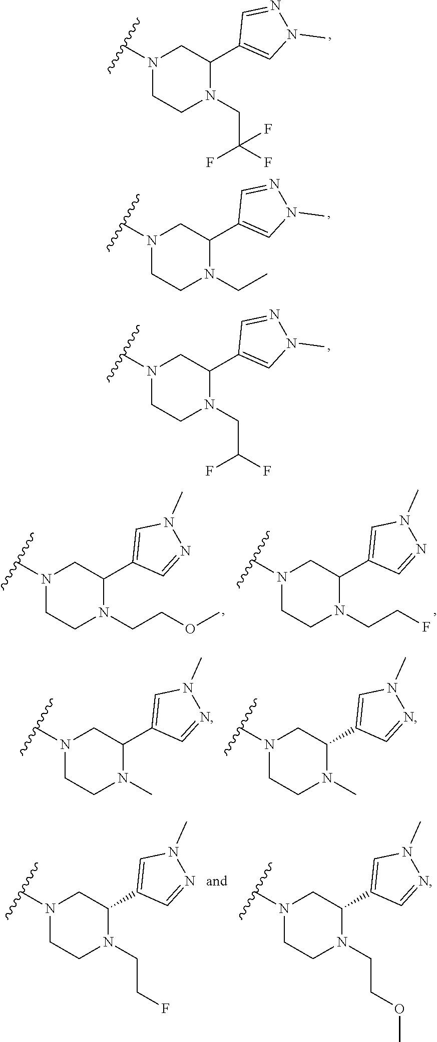
or a pharmaceutically acceptable salt thereof.

297. The compound according to any one of the preceding claims, wherein R²³ is selected from the group consisting of: 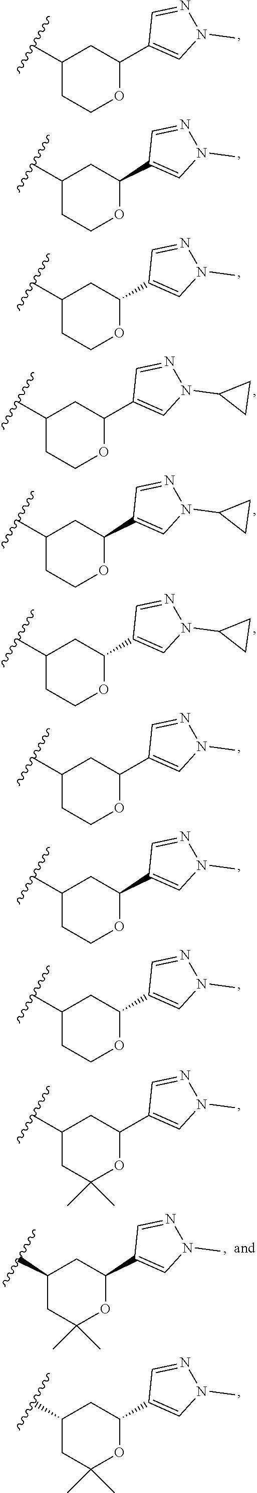
or a pharmaceutically acceptable salt thereof.

298. The compound according to any one of the preceding claims, wherein R²³ is selected from the group consisting of: 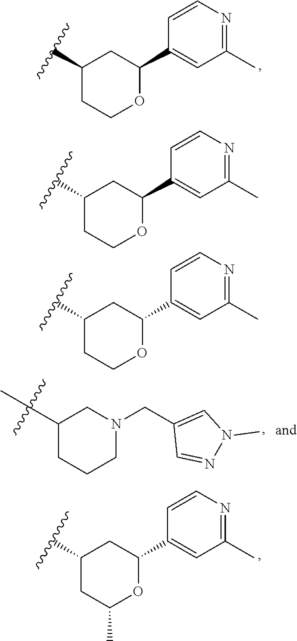
or a pharmaceutically acceptable salt thereof.

299. The compound according to any one of the preceding claims, or a pharmaceutically acceptable salt thereof, wherein R²³ is selected from the group consisting of:

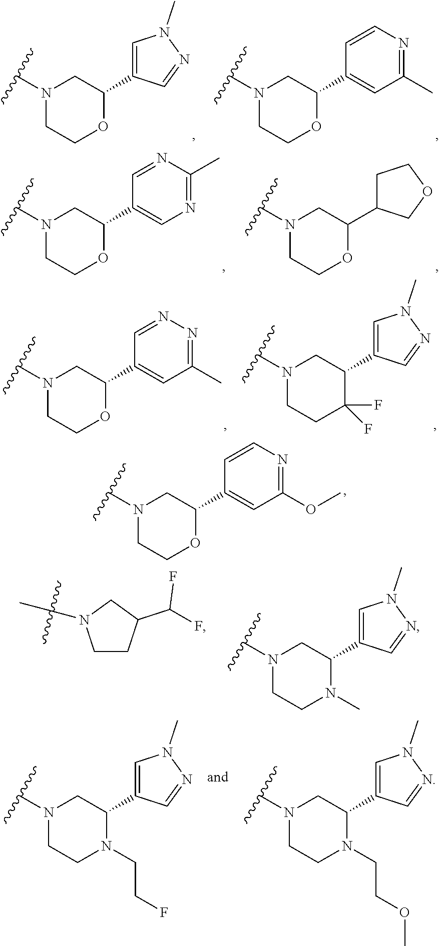

300. The compound according to any one of the preceding claims, wherein R²³ is selected from the group consisting of: 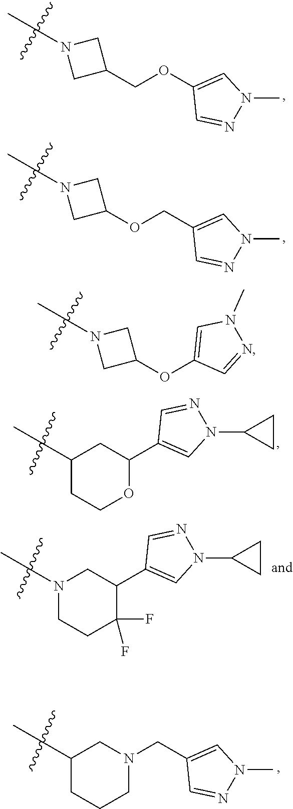
or a pharmaceutically acceptable salt thereof.

301. The compound according to any one of the preceding claims, wherein X³ is N, X⁴ is O, R⁴ is H, R⁶ is H, X¹⁰ is C(H)(R⁷), R⁷ is H and n is 1, or a pharmaceutically acceptable salt thereof.

302. The compound according to any one of the preceding claims, wherein X³ is CH, X⁴ is O, R⁴ is H, R⁶ is H, X¹⁰ is C(H)(R⁷), R⁷ is H and n is 1, or a pharmaceutically acceptable salt thereof.

303. The compound according to any one of the preceding claims, wherein X³ is N, X⁴ is a bond, R⁴ is H, R⁶ is H and n is O, or a pharmaceutically acceptable salt thereof.

304. The compound according to any one of the preceding claims, wherein R²³ is of Formula XI: 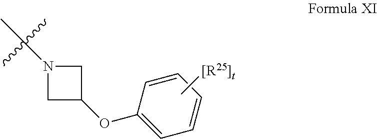
wherein
R²⁵ is individually selected from the group consisting of C₁₋₆ alkyl, —O— C₁₋₆ alkyl, halogen, C₁₋₆ haloalkyl and —CN; and
t is 0, 1, 2 or 3,
or a pharmaceutically acceptable salt thereof.

305. The compound according to any one of the preceding claims, wherein R²³ is of Formula XIX: 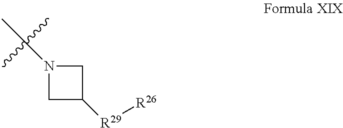
wherein
R²⁶ is a 5- or 6-membered heteroaryl optionally substituted optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃-6 heterocycloalkyl,
wherein the C₁₋₆ alkyl or C₁₋₆ haloalkyl is optionally substituted with —OH, and wherein the C₃₋₆ heterocycloalkyl is optionally substituted with 1 to 3 substituents individually selected from the group consisting of halogen, C₁₋₃ alkyl and —C(O)O(C₁₋₆ alkyl); and
R²⁹ is a bond, —O— or C₁₋₆ hydrocarbon chain wherein one methylene group is optionally replaced with —O—;
or a pharmaceutically acceptable salt thereof.

306. The compound according to any one of the preceding claims, wherein R²³ is of Formula XII: 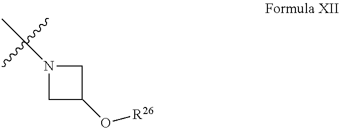
wherein
R²⁶ is a 5- or 6-membered heteroaryl optionally substituted optionally substituted with 1 to 3 substituents independently selected from the group consisting of halogen, C₁₋₆ alkyl, —O—C₁₋₆ alkyl, C₁₋₆ haloalkyl, C₁₋₆ alkoxy, C₁₋₆ haloalkoxy, —(C₁₋₃ alkyl)O(C₁₋₃ alkyl), —CN, C₂₋₄ alkenyl, C₃₋₆ cycloalkyl and C₃₋₆ heterocycloalkyl,
wherein the C₁₋₆ alkyl or C₁₋₆ haloalkyl is optionally substituted with —OH, and wherein the C₃₋₆ heterocycloalkyl is optionally substituted with 1 to 3 substituents individually selected from the group consisting of halogen, C₁₋₃ alkyl and —C(O)O(C₁₋₆ alkyl), or a pharmaceutically acceptable salt thereof.

307. The compound according to any one of the preceding claims, wherein R²⁶ is R⁵.

308. The compound according to any one of the preceding claims, wherein R²³ is 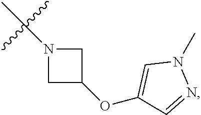 [SMILES: *N1CC(Oc2cnn(C)c2)C1]
or a pharmaceutically acceptable salt thereof.

309. The compound according to any one of the preceding claims, wherein R²³ is of Formula XIV: 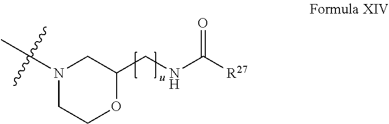
wherein
R²⁷ is C₁₋₆ alkyl; and
u is 1, 2 or 3,
or a pharmaceutically acceptable salt thereof.

310. The compound according to any one of the preceding claims, wherein R²³ is a 3 membered ring, or a pharmaceutically acceptable salt thereof.

311. The compound according to any one of the preceding claims, wherein X³ is C(R⁸); X⁴ is a bond; n is 0; X¹¹ is C(R⁴); X¹² is C(R⁶)(R^{6b}); and v is 0, or a pharmaceutically acceptable salt thereof.

312. The compound according to any one of the preceding claims, wherein X³ is C(H), X¹² is C(H)₂, X⁴ is a bond, X¹¹ is C(H), n is 0 and v is 0, or a pharmaceutically acceptable salt thereof.

313. The compound according to any one of the preceding claims, wherein R²³ is of Formula LXII: 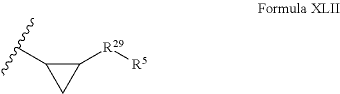
or a pharmaceutically acceptable salt thereof.

314. The compound according to any one of the preceding claims, wherein R²³ is of Formula LXIIa or Formula LXIIb: 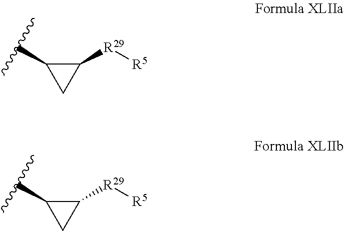
or a pharmaceutically acceptable salt thereof.

315. The compound according to any one of the preceding claims, wherein R²³ is 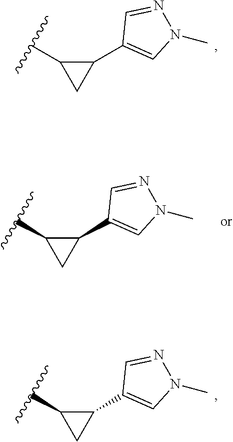
or a pharmaceutically acceptable salt thereof.

316. The compound according to any one of the preceding claims, wherein R²³ is of Formula XVII: 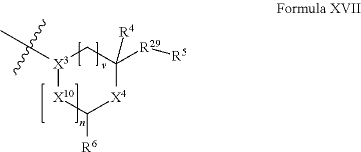
or a pharmaceutically acceptable salt thereof.

317. The compound according to any one of the preceding claims, wherein R²³ is of Formula XVIII: 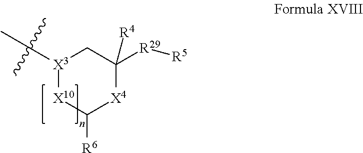
or a pharmaceutically acceptable salt thereof.

318. The compound according to any one of the preceding claims, wherein R²³ is of Formula X: 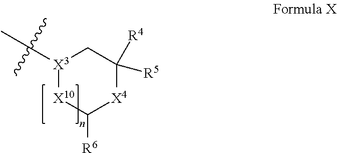
or a pharmaceutically acceptable salt thereof.

319. The compound according to any one of the preceding claims, wherein R²⁹ is a bond, or a pharmaceutically acceptable salt thereof.

320. The compound according to any one of the preceding claims, wherein R²⁹ is a C₁₋₃ alkoxy, or a pharmaceutically acceptable salt thereof.

321. The compound according to any one of the preceding claims, wherein R²⁹ is —O—C₁₋₃ alkyl, or a pharmaceutically acceptable salt thereof.

322. The compound according to any one of the preceding claims, wherein X¹¹ is C(R⁴), or a pharmaceutically acceptable salt thereof.

323. The compound according to any one of the preceding claims, wherein X¹¹ is N, or a pharmaceutically acceptable salt thereof.

324. The compound according to any one of the preceding claims, wherein X¹³ is CH₂, or a pharmaceutically acceptable salt thereof.

325. The compound according to any one of the preceding claims, wherein R⁴⁰ is H, or a pharmaceutically acceptable salt thereof.

326. The compound according to any one of the preceding claims, wherein R⁴¹ is H, or a pharmaceutically acceptable salt thereof.

327. The compound according to any one of the preceding claims, wherein R⁴⁰ is H and R⁴¹ is H, or a pharmaceutically acceptable salt thereof.

328. The compound according to any one of the preceding claims, wherein v is 1, or a pharmaceutically acceptable salt thereof.

329. The compound according to any one of the preceding claims, wherein v is 0, or a pharmaceutically acceptable salt thereof.

330. The compound according to any one of the preceding claims, wherein v is 2, or a pharmaceutically acceptable salt thereof.

331. The compound according to any one of the preceding claims, wherein R⁵ is C₃₋₆ cycloalkyl or C₃₋₆ heterocycloalkyl, and the C₃₋₆ cycloalkyl or C₃₋₆ heterocycloalkyl is optionally substituted with oxo (═O), or a pharmaceutically acceptable salt thereof.

332. The compound according to any one of the preceding claims, wherein R⁵ is C₁₋₆ alkyl, wherein the C₁₋₆ alkyl is optionally substituted with —N(H)C(O)R²⁴.

333. The compound according to any one of the preceding claims, wherein R⁹ is C₁₋₆ alkyl, —C(O)—C₁₋₆ alkyl or —C(O)—C₃₋₆ cycloalkyl, or a pharmaceutically acceptable salt thereof.

334. The compound according to any one of the preceding claims, wherein R²⁹ is a bond, or a pharmaceutically acceptable salt thereof.

335. The compound according to any one of the preceding claims, wherein R²⁹ is O, or a pharmaceutically acceptable salt thereof.

336. The compound according to any one of the preceding claims, wherein R³⁰ is H, or a pharmaceutically acceptable salt thereof.

337. The compound according to any one of the preceding claims, wherein R³⁰ is C₁₋₃ alkyl, such as C₁ alkyl, or a pharmaceutically acceptable salt thereof.

338. The compound according to any one of the preceding claims, wherein R³¹ is H, or a pharmaceutically acceptable salt thereof.

339. The compound according to any one of the preceding claims, wherein R³¹ is C₁₋₃ alkyl, such as C₁ alkyl, or a pharmaceutically acceptable salt thereof.

340. The compound according to any one of the preceding claims, wherein, R³⁰ is C₁₋₃ alkyl, such as C₁ alkyl, and R³¹ is H, or a pharmaceutically acceptable salt thereof.

341. The compound according to any one of the preceding claims, wherein R³⁰ is C₁₋₃ alkyl, such as C₁ alkyl, and R³¹ is C₁₋₃ alkyl, such as C₁ alkyl, or a pharmaceutically acceptable salt thereof.

342. The compound according to any one of the preceding claims, wherein v is 1, or a pharmaceutically acceptable salt thereof.

343. The compound according to any one of the preceding claims, wherein v is 2, or a pharmaceutically acceptable salt thereof.

344. The compound according to any one of the preceding claims, wherein n is 0, or a pharmaceutically acceptable salt thereof.

345. The compound according to any one of the preceding claims, wherein n is 1, or a pharmaceutically acceptable salt thereof.

346. The compound according to any one of the preceding claims, wherein X¹² is CH₂, R²⁹ is a bond and R⁵ is pyrazolyl optionally substituted with C₁₋₆ alkyl or C₃₋₆ cycloalkyl, or a pharmaceutically acceptable salt thereof.

347. The compound according to any one of the preceding claims, wherein X¹² is CH₂, R²⁹ is a bond and R⁵ is pyrazolyl substituted with C₃₋₆ cycloalkyl, such as cyclopropyl, or a pharmaceutically acceptable salt thereof.

348. The compound according to any one of the preceding claims, wherein the halogen is F, or a pharmaceutically acceptable salt thereof.

349. The compound according to any one of the preceding claims, wherein the halogen is Cl, or a pharmaceutically acceptable salt thereof.

350. The compound according to claim 1, wherein the compound is of Formula LXI, wherein
X¹ is N;
X² is N;
X¹⁴ is N;
R¹ is —CF₃,
R² is C₁₋₆ alkyl, such as CH₃,
R³ is of Formula IV: 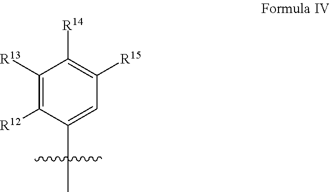
wherein
R¹² is H, halogen, C₁₋₃ alkyl or C₁₋₃ haloalkyl;
R¹³ is H, halogen, C₁₋₃ alkyl or C₁₋₃ haloalkyl;
R¹⁴ is H, halogen, C₁₋₃ alkyl or C₁₋₃ haloalkyl; and
R¹⁵ is H, halogen, C₁₋₃ alkyl or C₁₋₃ haloalkyl,
or a pharmaceutically acceptable salt thereof.

351. The compound according to claim 350, wherein R¹² is halogen, R¹³ is H, R¹⁴ is halogen and R¹⁵ is H, or a pharmaceutically acceptable salt thereof.

352. The compound according to claim 351, wherein R¹² is F and R¹⁴ is Cl, or a pharmaceutically acceptable salt thereof.

353. The compound according to any one of claims 350 to 352, wherein R²³ is of Formula XXXIV: 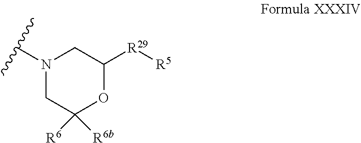
wherein
R⁶ is H, halogen or C₁₋₃ alkyl;
R^{6b} is H, halogen or C₁₋₃ alkyl; and
R²⁹ is a bond,
or a pharmaceutically acceptable salt thereof.

354. The compound according to claim 353, wherein R⁶ is H and R^{6b} is H.

355. The compound according to any one of claims 350 to 354, wherein R⁵ is of Formula XXII: 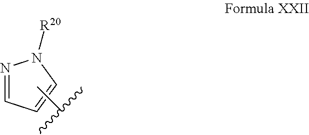
wherein
R²⁰ is C₁₋₃ alkyl or C₃₋₆ cycloalkyl, or a pharmaceutically acceptable salt thereof.

356. The compound according to any one of claims 350 to 355, wherein R⁵ is of Formula VII: 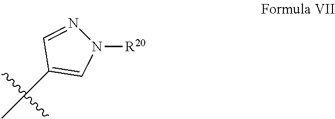
wherein
R²⁰ is C₁₋₃ alkyl or C₃₋₆ cycloalkyl, or a pharmaceutically acceptable salt thereof.

357. The compound according to any one of claims 350 to 356, wherein R²³ is 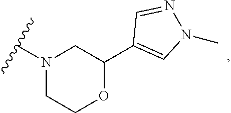 [SMILES: *N1CCOC(c2cnn(C)c2)C1]
or a pharmaceutically acceptable salt thereof.

358. The compound according to any one of claims 350 to 356, wherein R²³ is 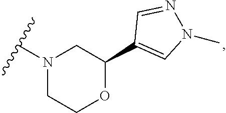 [SMILES: *N1CCO[C@H](c2cnn(C)c2)C1]
or a pharmaceutically acceptable salt thereof.

359. The compound according to claim 1, wherein the compound is of Formula LXI, wherein
X¹ is N;
X² is N;
X¹⁴ is N;
R¹ is —CH₃,
R² is C₁₋₆ alkyl, such as CH₃,
R³ is of Formula IV: 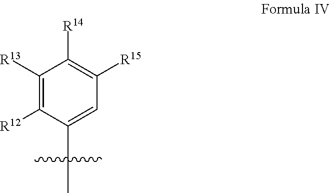
wherein
R¹² is H, halogen, C₁₋₃ alkyl or C₁₋₃ haloalkyl;
R¹³ is H, halogen, C₁₋₃ alkyl or C₁₋₃ haloalkyl;
R¹⁴ is H, halogen, C₁₋₃ alkyl, or C₁₋₃ haloalkyl; and
R¹⁵ is H, halogen, C₁₋₃ alkyl or C₁₋₃ haloalkyl,
or a pharmaceutically acceptable salt thereof.

360. The compound according to claim 359, wherein R¹² is halogen, R¹³ is H, R¹⁴ is haloalkyl and R¹⁵ is H, or a pharmaceutically acceptable salt thereof.

361. The compound according to claim 360, wherein R¹² is F and R¹⁴ is CF₃, or a pharmaceutically acceptable salt thereof.

362. The compound according to any one of claims 359 to 361, wherein R²³ is of Formula XXXIV: 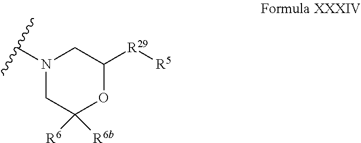
wherein
R⁶ is H, halogen or C₁₋₃ alkyl;
R^{6b} is H, halogen or C₁₋₃ alkyl; and
R²⁹ is a bond,
or a pharmaceutically acceptable salt thereof.

363. The compound according to claim 362, wherein R⁶ is H and R^{6b} is H.

364. The compound according to any one of claims 359 to 363, wherein R⁵ is Formula VIII: 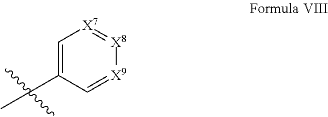
wherein
X⁷ is N or CH;
X³ is N or C(R²¹);
X⁹ is N or C(R²²);
R²¹ is C₁₋₃ alkyl, such as —CH₃; and
R²² is C₁₋₃ alkyl, such as —CH₃,
or a pharmaceutically acceptable salt thereof.

365. The compound according to any one of claims 359 to 364, wherein R⁵ is 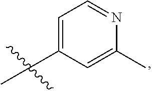 [SMILES: *c1ccnc(C)c1]
or a pharmaceutically acceptable salt thereof.

366. The compound according to any one of claims 359 to 365, wherein R²³ is 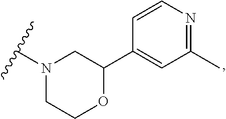 [SMILES: *N1CCOC(c2ccnc(C)c2)C1]
or a pharmaceutically acceptable salt thereof.

367. The compound according to any one of claims 359 to 366, wherein R²³ is 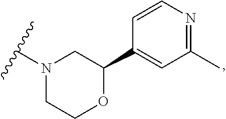 [SMILES: *N1CCO[C@H](c2ccnc(C)c2)C1]
or a pharmaceutically acceptable salt thereof.

368. The compound according to any one of the preceding claims, wherein the compound is selected from the group consisting of:
8-(4-chloro-2-fluorophenyl)-3-methyl-6-[(2R)-2-(1-methyl-1H-pyrazol-4-yl)morpholin-4-yl]-3H,4H-pyrimido[5,4-d][1,3]diazin-4-one;
8-(4-chloro-2-fluorophenyl)-3-methyl-6-[(2S)-2-(1-methyl-1H-pyrazol-4-yl)morpholin-4-yl]-3H,4H-pyrimido[5,4-d][1,3]diazin-4-one;
8-(4-chloro-2-fluorophenyl)-2,3-dimethyl-6-[(2R)-2-(1-methyl-1H-pyrazol-4-yl)morpholin-4-yl]-3H,4H-pyrimido[5,4-d][1,3]diazin-4-one;
8-(4-chloro-2-fluorophenyl)-2,3-dimethyl-6-[(2S)-2-(1-methyl-1H-pyrazol-4-yl)morpholin-4-yl]-3H,4H-pyrimido[5,4-d][1,3]diazin-4-one;
8-(4-chloro-2-fluorophenyl)-6-[(2R)-2-(1-cyclopropyl-1H-pyrazol-4-yl)morpholin-4-yl]-2,3-dimethyl-3H,4H-pyrimido[5,4-d][1,3]diazin-4-one;
8-(4-chloro-2-fluorophenyl)-6-[(2S)-2-(1-cyclopropyl-1H-pyrazol-4-yl)morpholin-4-yl]-2,3-dimethyl-3H,4H-pyrimido[5,4-d][1,3]diazin-4-one;
8-(4-chloro-2-fluorophenyl)-2,3-dimethyl-6-[(2R)-2-(2-methylpyridin-4-yl)morpholin-4-yl]-3H,4H-pyrimido[5,4-d][1,3]diazin-4-one;
8-(4-chloro-2-fluorophenyl)-2,3-dimethyl-6-[(2S)-2-(2-methylpyridin-4-yl)morpholin-4-yl]-3H,4H-pyrimido[5,4-d][1,3]diazin-4-one;
8-(4-chloro-2-fluorophenyl)-2,3-dimethyl-6-[(2R)-2-(2-methylpyrimidin-5-yl)morpholin-4-yl]-3H,4H-pyrimido[5,4-d][1,3]diazin-4-one;
8-(4-chloro-2-fluorophenyl)-2,3-dimethyl-6-[(2S)-2-(2-methylpyrimidin-5-yl)morpholin-4-yl]-3H,4H-pyrimido[5,4-d][1,3]diazin-4-one;
8-(4-chloro-2-fluorophenyl)-2,3-dimethyl-6-[(2R)-2-(6-methylpyridazin-4-yl)morpholin-4-yl]-3H,4H-pyrimido[5,4-d][1,3]diazin-4-one;
8-(4-chloro-2-fluorophenyl)-2,3-dimethyl-6-[(2S)-2-(6-methylpyridazin-4-yl)morpholin-4-yl]-3H,4H-pyrimido[5,4-d][1,3]diazin-4-one;
8-(4-chloro-2-fluorophenyl)-6-[(3R)-4,4-difluoro-3-(1-methyl-1H-pyrazol-4-yl)piperidin-1-yl]-2,3-dimethyl-3H,4H-pyrimido[5,4-d][1,3]diazin-4-one;
8-(4-chloro-2-fluorophenyl)-6-[(3S)-4,4-difluoro-3-(1-methyl-1H-pyrazol-4-yl)piperidin-1-yl]-2,3-dimethyl-3H,4H-pyrimido[5,4-d][1,3]diazin-4-one;
8-(4-chloro-2-fluorophenyl)-2,3-dimethyl-6-[(2S,4R)-2-(1-methyl-1H-pyrazol-4-yl)oxan-4-yl]-3H,4H-pyrimido[5,4-d][1,3]diazin-4-one;
8-(4-chloro-2-fluorophenyl)-2,3-dimethyl-6-[(2R,4S)-2-(1-methyl-1H-pyrazol-4-yl)oxan-4-yl]-3H,4H-pyrimido[5,4-d][1,3]diazin-4-one;
8-(4-chloro-2-fluorophenyl)-2,3-dimethyl-6-[(2R,4R)-2-(1-methyl-1H-pyrazol-4-yl)oxan-4-yl]-3H,4H-pyrimido[5,4-d][1,3]diazin-4-one;
8-(4-chloro-2-fluorophenyl)-2,3-dimethyl-6-[(2S,4S)-2-(1-methyl-1H-pyrazol-4-yl)oxan-4-yl]-3H,4H-pyrimido[5,4-d][1,3]diazin-4-one;
8-(4-chloro-2-fluorophenyl)-6-[(2S,4R)-2-(1-cyclopropyl-1H-pyrazol-4-yl)oxan-4-yl]-2,3-dimethyl-3H,4H-pyrimido[5,4-d][1,3]diazin-4-one;
8-(4-chloro-2-fluorophenyl)-6-[(2R,4S)-2-(1-cyclopropyl-1H-pyrazol-4-yl)oxan-4-yl]-2,3-dimethyl-3H,4H-pyrimido[5,4-d][1,3]diazin-4-one;
8-(4-chloro-2-fluorophenyl)-6-[(2R,4R)-2-(1-cyclopropyl-1H-pyrazol-4-yl)oxan-4-yl]-2,3-dimethyl-3H,4H-pyrimido[5,4-d][1,3]diazin-4-one;
8-(4-chloro-2-fluorophenyl)-6-[(2S,4S)-2-(1-cyclopropyl-1H-pyrazol-4-yl)oxan-4-yl]-2,3-dimethyl-3H,4H-pyrimido[5,4-d][1,3]diazin-4-one;
8-(4-chloro-2-fluorophenyl)-2,3-dimethyl-6-[(2S,4R)-2-(2-methylpyridin-4-yl)oxan-4-yl]-3H,4H-pyrimido[5,4-d][1,3]diazin-4-one;
8-(4-chloro-2-fluorophenyl)-2,3-dimethyl-6-[(2R,4S)-2-(2-methylpyridin-4-yl)oxan-4-yl]-3H,4H-pyrimido[5,4-d][1,3]diazin-4-one;
8-(4-chloro-2-fluorophenyl)-2,3-dimethyl-6-[(2R,4R)-2-(2-methylpyridin-4-yl)oxan-4-yl]-3H,4H-pyrimido[5,4-d][1,3]diazin-4-one;
8-(4-chloro-2-fluorophenyl)-2,3-dimethyl-6-[(2S,4S)-2-(2-methylpyridin-4-yl)oxan-4-yl]-3H,4H-pyrimido[5,4-d][1,3]diazin-4-one;
8-(4-chloro-2-fluorophenyl)-2,3-dimethyl-6-[(2S,4R)-2-(2-methylpyrimidin-5-yl)oxan-4-yl]-3H,4H-pyrimido[5,4-d][1,3]diazin-4-one;
8-(4-chloro-2-fluorophenyl)-2,3-dimethyl-6-[(2R,4S)-2-(2-methylpyrimidin-5-yl)oxan-4-yl]-3H,4H-pyrimido[5,4-d][1,3]diazin-4-one;
8-(4-chloro-2-fluorophenyl)-2,3-dimethyl-6-[(2R,4R)-2-(2-methylpyrimidin-5-yl)oxan-4-yl]-3H,4H-pyrimido[5,4-d][1,3]diazin-4-one;
8-(4-chloro-2-fluorophenyl)-2,3-dimethyl-6-[(2S,4S)-2-(2-methylpyrimidin-5-yl)oxan-4-yl]-3H,4H-pyrimido[5,4-d][1,3]diazin-4-one;
8-{6,6-difluorospiro[3.3]heptan-2-yl}-2,3-dimethyl-6-[(2R)-2-(1-methyl-1H-pyrazol-4-yl)morpholin-4-yl]-3H,4H-pyrimido[5,4-d][1,3]diazin-4-one;
8-{6,6-difluorospiro[3.3]heptan-2-yl}-2,3-dimethyl-6-[(2S)-2-(1-methyl-1H-pyrazol-4-yl)morpholin-4-yl]-3H,4H-pyrimido[5,4-d][1,3]diazin-4-one;
2,3-dimethyl-6-[(2R)-2-(1-methyl-1H-pyrazol-4-yl)morpholin-4-yl]-8-[(1r,3r)-3-(trifluoromethyl)cyclobutyl]-3H,4H-pyrimido[5,4-d][1,3]diazin-4-one;
2,3-dimethyl-6-[(2S)-2-(1-methyl-1H-pyrazol-4-yl)morpholin-4-yl]-8-[(1r,3r)-3-(trifluoromethyl)cyclobutyl]-3H,4H-pyrimido[5,4-d][1,3]diazin-4-one;
2,3-dimethyl-6-[(2R)-2-(1-methyl-1H-pyrazol-4-yl)morpholin-4-yl]-8-[3-(trifluoromethyl)bicyclo[1.1.1]pentan-1-yl]-3H,4H-pyrimido[5,4-d][1,3]diazin-4-one;
2,3-dimethyl-6-[(2S)-2-(1-methyl-1H-pyrazol-4-yl)morpholin-4-yl]-8-[3-(trifluoromethyl)bicyclo[1.1.1]pentan-1-yl]-3H,4H-pyrimido[5,4-d][1,3]diazin-4-one;
2,3-dimethyl-6-[(2R)-2-(1-methyl-1H-pyrazol-4-yl)morpholin-4-yl]-8-[6-(trifluoromethyl)pyridin-3-yl]-3H,4H-pyrimido[5,4-d][1,3]diazin-4-one;
2,3-dimethyl-6-[(2S)-2-(1-methyl-1H-pyrazol-4-yl)morpholin-4-yl]-8-[6-(trifluoromethyl)pyridin-3-yl]-3H,4H-pyrimido[5,4-d][1,3]diazin-4-one;
8-(4,4-difluorocyclohexyl)-2,3-dimethyl-6-[(2R)-2-(1-methyl-1H-pyrazol-4-yl)morpholin-4-yl]-3H,4H-pyrimido[5,4-d][1,3]diazin-4-one;
8-(4,4-difluorocyclohexyl)-2,3-dimethyl-6-[(2S)-2-(1-methyl-1H-pyrazol-4-yl)morpholin-4-yl]-3H,4H-pyrimido[5,4-d][1,3]diazin-4-one;
8-(2,4-difluorophenyl)-2,3-dimethyl-6-[(2R)-2-(1-methyl-1H-pyrazol-4-yl)morpholin-4-yl]-3H,4H-pyrimido[5,4-d][1,3]diazin-4-one;
8-(2,4-difluorophenyl)-2,3-dimethyl-6-[(2S)-2-(1-methyl-1H-pyrazol-4-yl)morpholin-4-yl]-3H,4H-pyrimido[5,4-d][1,3]diazin-4-one;
2,3-dimethyl-6-[(2R)-2-(1-methyl-1H-pyrazol-4-yl)morpholin-4-yl]-8-(2,3,4-trifluorophenyl)-3H,4H-pyrimido[5,4-d][1,3]diazin-4-one;
2,3-dimethyl-6-[(2S)-2-(1-methyl-1H-pyrazol-4-yl)morpholin-4-yl]-8-(2,3,4-trifluorophenyl)-3H,4H-pyrimido[5,4-d][1,3]diazin-4-one;
2,3-dimethyl-6-[(2R)-2-(1-methyl-1H-pyrazol-4-yl)morpholin-4-yl]-8-(2,4,5-trifluorophenyl)-3H,4H-pyrimido[5,4-d][1,3]diazin-4-one;
2,3-dimethyl-6-[(2S)-2-(1-methyl-1H-pyrazol-4-yl)morpholin-4-yl]-8-(2,4,5-trifluorophenyl)-3H,4H-pyrimido[5,4-d][1,3]diazin-4-one;
2,3-dimethyl-6-[(2R)-2-(1-methyl-1H-pyrazol-4-yl)morpholin-4-yl]-8-(3,4,5-trifluorophenyl)-3H,4H-pyrimido[5,4-d][1,3]diazin-4-one;
2,3-dimethyl-6-[(2S)-2-(1-methyl-1H-pyrazol-4-yl)morpholin-4-yl]-8-(3,4,5-trifluorophenyl)-3H,4H-pyrimido[5,4-d][1,3]diazin-4-one;
8-(3,4-difluorophenyl)-2,3-dimethyl-6-[(2R)-2-(1-methyl-1H-pyrazol-4-yl)morpholin-4-yl]-3H,4H-pyrimido[5,4-d][1,3]diazin-4-one;
8-(3,4-difluorophenyl)-2,3-dimethyl-6-[(2S)-2-(1-methyl-1H-pyrazol-4-yl)morpholin-4-yl]-3H,4H-pyrimido[5,4-d][1,3]diazin-4-one;
2,3-dimethyl-6-[(2R)-2-(1-methyl-1H-pyrazol-4-yl)morpholin-4-yl]-8-[(1r,4r)-4-methylcyclohexyl]-3H,4H-pyrimido[5,4-d][1,3]diazin-4-one;
2,3-dimethyl-6-[(2S)-2-(1-methyl-1H-pyrazol-4-yl)morpholin-4-yl]-8-[(1r,4r)-4-methylcyclohexyl]-3H,4H-pyrimido[5,4-d][1,3]diazin-4-one;
8-[2-fluoro-4-(trifluoromethyl)phenyl]-2,3-dimethyl-6-[(2R)-2-(1-methyl-1H-pyrazol-4-yl)morpholin-4-yl]-3H,4H-pyrimido[5,4-d][1,3]diazin-4-one;
8-[2-fluoro-4-(trifluoromethyl)phenyl]-2,3-dimethyl-6-[(2S)-2-(1-methyl-1H-pyrazol-4-yl)morpholin-4-yl]-3H,4H-pyrimido[5,4-d][1,3]diazin-4-one;
2,3-dimethyl-6-[(2R)-2-(1-methyl-1H-pyrazol-4-yl)morpholin-4-yl]-8-(oxan-4-yl)-3H,4H,4aH,8aH-[1,3]diazino[5,4-d]pyrimidin-4-one;
2,3-dimethyl-6-[(2S)-2-(1-methyl-1H-pyrazol-4-yl)morpholin-4-yl]-8-(oxan-4-yl)-3H,4H,4aH,8aH-[1,3]diazino[5,4-d]pyrimidin-4-one;
8-(4,4-difluoropiperidin-1-yl)-2,3-dimethyl-6-[(2R)-2-(1-methyl-1H-pyrazol-4-yl)morpholin-4-yl]-3H,4H,4aH,8aH-[1,3]diazino[5,4-d]pyrimidin-4-one;
8-(4,4-difluoropiperidin-1-yl)-2,3-dimethyl-6-[(2S)-2-(1-methyl-1H-pyrazol-4-yl)morpholin-4-yl]-3H,4H,4aH,8aH-[1,3]diazino[5,4-d]pyrimidin-4-one;
8-(4-chloro-2-fluorophenyl)-2,3-dimethyl-6-{3-[(1-methyl-1H-pyrazol-4-yl)oxy]azetidin-1-yl}-3H,4H,4aH,8aH-[1,3]diazino[5,4-d]pyrimidin-4-one;
8-(4-chloro-2-fluorophenyl)-2,3-dimethyl-6-[(3R)-4-methyl-3-(1-methyl-1H-pyrazol-4-yl)piperazin-1-yl]-3H,4H-pyrimido[5,4-d][1,3]diazin-4-one;
8-(4-chloro-2-fluorophenyl)-2,3-dimethyl-6-[(3S)-4-methyl-3-(1-methyl-1H-pyrazol-4-yl)piperazin-1-yl]-3H,4H-pyrimido[5,4-d][1,3]diazin-4-one;
8-(4-chloro-2-fluorophenyl)-6-[(3R)-4-(2-fluoroethyl)-3-(1-methyl-1H-pyrazol-4-yl)piperazin-1-yl]-2,3-dimethyl-3H,4H-pyrimido[5,4-d][1,3]diazin-4-one;
8-(4-chloro-2-fluorophenyl)-6-[(3S)-4-(2-fluoroethyl)-3-(1-methyl-1H-pyrazol-4-yl)piperazin-1-yl]-2,3-dimethyl-3H,4H-pyrimido[5,4-d][1,3]diazin-4-one;
8-(4,4-difluorocyclohexyl)-2,3-dimethyl-6-(2-(1-methyl-1H-pyrazol-4-yl)morpholino)pyrimido[5,4-d]pyrimidin-4 (3H)-one; 2,3-dimethyl-6-(2-(1-methyl-1H-pyrazol-4-yl)morpholino)-8-((1s,4s)-4-methylcyclohexyl)pyrimido[5,4-d]pyrimidin-4 (3H)-one;
2,3-dimethyl-6-(2-(1-methyl-1H-pyrazol-4-yl)morpholino)-8-((1r,4r)-4-methylcyclohexyl)pyrimido[5,4-d]pyrimidin-4 (3H)-one;
8-(4-chloro-2-fluorophenyl)-6-(4,4-difluoro-3-(1-methyl-1H-pyrazol-4-yl)piperidin-1-yl)-2,3-dimethylpyrimido[5,4-d]pyrimidin-4 (3H)-one;
8-(4-chloro-2-fluorophenyl)-6-((2R,4S)-2-(1-cyclopropyl-1H-pyrazol-4-yl)tetrahydro-2H-pyran-4-yl)-2,3-dimethylpyrimido[5,4-d]pyrimidin-4 (3H)-one;
8-(4-chloro-2-fluorophenyl)-2,3-dimethyl-6-(3-(((1-methyl-1H-pyrazol-4-yl)oxy)methyl)azetidin-1-yl)pyrimido[5,4-d]pyrimidin-4 (3H)-one;
8-(4-chloro-2-fluorophenyl)-2,3-dimethyl-6-(3-((1-methyl-1H-pyrazol-4-yl)oxy)azetidin-1-yl)pyrimido[5,4-d]pyrimidin-4 (3H)-one;
8-(4-chloro-2-fluorophenyl)-2,3-dimethyl-6-(2-(1-methyl-1H-pyrazol-4-yl)tetrahydro-2H-pyran-4-yl)pyrimido[5,4-d]pyrimidin-4 (3H)-one;
8-(4-chloro-2-fluorophenyl)-2,3-dimethyl-6-(3-((1-methyl-1H-pyrazol-4-yl)methoxy)azetidin-1-yl)pyrimido[5,4-d]pyrimidin-4 (3H)-one;
(R)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one;
(S)-6-(2-(1-cyclopropyl-1H-pyrazol-4-yl)morpholino)-2,3-dimethyl-8-(6-(trifluoromethyl)pyridin-3-yl)pyrimido[5,4-d]pyrimidin-4 (3H)-one;
(R)-6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one;
(S)-6-(2-(1-cyclopropyl-1H-pyrazol-4-yl)morpholino)-8-(2-fluoro-4-(trifluoromethyl)phenyl)-2,3-dimethylpyrimido[5,4-d]pyrimidin-4 (3H)-one;
(R)-8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one;
(S)-8-(2-fluoro-4-(trifluoromethyl)phenyl)-2,3-dimethyl-6-(2-(2-methylpyridin-4-yl)morpholino)pyrimido[5,4-d]pyrimidin-4 (3H)-one;
(R)-2,3-dimethyl-6-(2-(2-methylpyridin-4-yl)morpholino)-8-(6-(trifluoromethyl)pyridin-3-yl)pyrimido[5,4-d]pyrimidin-4 (3H)-one;
(S)-2,3-dimethyl-6-(2-(2-methylpyridin-4-yl)morpholino)-8-(6-(trifluoromethyl)pyridin-3-yl)pyrimido[5,4-d]pyrimidin-4 (3H)-one;
(R)-8-(4-chloro-2-fluorophenyl)-2,3-dimethyl-6-(2-(2-methylpyridin-4-yl)morpholino)pyrimido[5,4-d]pyrimidin-4 (3H)-one;
(S)-8-(4-chloro-2-fluorophenyl)-2,3-dimethyl-6-(2-(2-methylpyridin-4-yl)morpholino)pyrimido[5,4-d]pyrimidin-4 (3H)-one;
8-(4-chloro-2-fluorophenyl)-6-((2S,4R)-2-(1-cyclopropyl-1H-pyrazol-4-yl)tetrahydro-2H-pyran-4-yl)-2,3-dimethylpyrimido[5,4-d]pyrimidin-4 (3H)-one;
8-(4-chloro-2-fluorophenyl)-6-((2R,4S)-2-(1-cyclopropyl-1H-pyrazol-4-yl)tetrahydro-2H-pyran-4-yl)-2,3-dimethylpyrimido[5,4-d]pyrimidin-4 (3H)-one;
(R)-8-(2-fluoro-4-(trifluoromethyl)phenyl)-2,3-dimethyl-6-(2-(1-methyl-1H-pyrazol-4-yl)morpholino)pyrimido[5,4-d]pyrimidin-4 (3H)-one;
(S)-8-(2-fluoro-4-(trifluoromethyl)phenyl)-2,3-dimethyl-6-(2-(1-methyl-1H-pyrazol-4-yl)morpholino)pyrimido[5,4-d]pyrimidin-4 (3H)-one;
(R)-8-(4-chloro-2-fluorophenyl)-6-(2-(1-cyclopropyl-1H-pyrazol-4-yl)morpholino)-2,3-dimethylpyrimido[5,4-d]pyrimidin-4 (3H)-one;
(S)-8-(4-chloro-2-fluorophenyl)-6-(2-(1-cyclopropyl-1H-pyrazol-4-yl)morpholino)-2,3-dimethylpyrimido[5,4-d]pyrimidin-4 (3H)-one;
(R)-2,3-dimethyl-6-(2-(1-methyl-1H-pyrazol-4-yl)morpholino)-8-(2,4,5-trifluorophenyl)pyrimido[5,4-d]pyrimidin-4 (3H)-one;
(S)-2,3-dimethyl-6-(2-(1-methyl-1H-pyrazol-4-yl)morpholino)-8-(2,4,5-trifluorophenyl)pyrimido[5,4-d]pyrimidin-4 (3H)-one;
(R)-2,3-dimethyl-6-(2-(1-methyl-1H-pyrazol-4-yl)morpholino)-8-(6-(trifluoromethyl)pyridin-3-yl)pyrimido[5,4-d]pyrimidin-4 (3H)-one;
(S)-2,3-dimethyl-6-(2-(1-methyl-1H-pyrazol-4-yl)morpholino)-8-(6-(trifluoromethyl)pyridin-3-yl)pyrimido[5,4-d]pyrimidin-4 (3H)-one;
8-(4-chloro-2-fluorophenyl)-2,3-dimethyl-6-(4-methyl-3-(1-methyl-1H-pyrazol-4-yl)piperazin-1-yl)pyrimido[5,4-d]pyrimidin-4 (3H)-one;
8-(4-chloro-2-fluorophenyl)-6-(4-ethyl-3-(1-methyl-1H-pyrazol-4-yl)piperazin-1-yl)-2,3-dimethylpyrimido[5,4-d]pyrimidin-4 (3H)-one;
8-(4-chloro-2-fluorophenyl)-6-(4-(2-fluoroethyl)-3-(1-methyl-1H-pyrazol-4-yl)piperazin-1-yl)-2,3-dimethylpyrimido[5,4-d]pyrimidin-4 (3H)-one;
8-(4-chloro-2-fluorophenyl)-6-(4-(2,2-difluoroethyl)-3-(1-methyl-1H-pyrazol-4-yl)piperazin-1-yl)-2,3-dimethylpyrimido[5,4-d]pyrimidin-4 (3H)-one;
8-(4-chloro-2-fluorophenyl)-2,3-dimethyl-6-(3-(1-methyl-1H-pyrazol-4-yl)-4-(2,2,2-trifluoroethyl)piperazin-1-yl)pyrimido[5,4-d]pyrimidin-4 (3H)-one;
8-(4-chloro-2-fluorophenyl)-6-(4-(2-methoxyethyl)-3-(1-methyl-1H-pyrazol-4-yl)piperazin-1-yl)-2,3-dimethylpyrimido[5,4-d]pyrimidin-4 (3H)-one;
8-(4-chloro-2-fluorophenyl)-2,3-dimethyl-6-(2-(2-methylpyrimidin-5-yl)morpholino)pyrimido[5,4-d]pyrimidin-4 (3H)-one;
8-(4-chloro-2-fluorophenyl)-2,3-dimethyl-6-(2-(1-methyl-1H-pyrazol-3-yl)morpholino)pyrimido[5,4-d]pyrimidin-4 (3H)-one;
8-(4-chloro-2-fluorophenyl)-2,3-dimethyl-6-((2R,4S)-2-(2-methylpyrimidin-5-yl)tetrahydro-2H-pyran-4-yl)pyrimido[5,4-d]pyrimidin-4 (3H)-one;
2,3-dimethyl-6-((2S,4R)-2-(1-methyl-1H-pyrazol-4-yl)tetrahydro-2H-pyran-4-yl)-8-(2,4,5-trifluorophenyl)pyrimido[5,4-d]pyrimidin-4 (3H)-one;
2,3-dimethyl-6-((2R,4S)-2-(1-methyl-1H-pyrazol-4-yl)tetrahydro-2H-pyran-4-yl)-8-(2,4,5-trifluorophenyl)pyrimido[5,4-d]pyrimidin-4 (3H)-one;
6-(2-(1H-pyrazol-4-yl)morpholino)-8-(4-chloro-2-fluorophenyl)-2,3-dimethylpyrimido[5,4-d]pyrimidin-4 (3H)-one;
8-(4-chloro-2-fluorophenyl)-2,3-dimethyl-6-(2-(1-(oxetan-3-yl)-1H-pyrazol-4-yl)morpholino)pyrimido[5,4-d]pyrimidin-4 (3H)-one;
6-(2-(1-(azetidin-3-yl)-1H-pyrazol-4-yl)morpholino)-8-(4-chloro-2-fluorophenyl)-2,3-dimethylpyrimido[5,4-d]pyrimidin-4 (3H)-one;
8-(4-chloro-2-fluorophenyl)-2,3-dimethyl-6-(2-(1-(1-methylazetidin-3-yl)-1H-pyrazol-4-yl)morpholino)pyrimido[5,4-d]pyrimidin-4 (3H)-one;
8-(4-chloro-2-fluorophenyl)-2,3-dimethyl-6-(2-(5-methyl-1,3,4-oxadiazol-2-yl)morpholino)pyrimido[5,4-d]pyrimidin-4 (3H)-one;
8-(4-chloro-2-fluorophenyl)-2,3-dimethyl-6-(2-(1-methyl-1H-pyrazol-5-yl)morpholino)pyrimido[5,4-d]pyrimidin-4 (3H)-one;
8-(4-chloro-2-fluorophenyl)-2,3-dimethyl-6-(2-(4-methylthiazol-2-yl)morpholino)pyrimido[5,4-d]pyrimidin-4 (3H)-one;
8-(4-chloro-2-fluorophenyl)-2,3-dimethyl-6-(2-(3-methyl-1H-1,2,4-triazol-5-yl)morpholino)pyrimido[5,4-d]pyrimidin-4 (3H)-one;
6-(2-(1H-pyrazol-3-yl)morpholino)-8-(4-chloro-2-fluorophenyl)-2,3-dimethylpyrimido[5,4-d]pyrimidin-4 (3H)-one;
8-(4-chloro-2-fluorophenyl)-2,3-dimethyl-6-(2-(thiazol-2-yl)morpholino)pyrimido[5,4-d]pyrimidin-4 (3H)-one;
4-(4-(4-chloro-2-fluorophenyl)-6,7-dimethyl-8-oxo-7,8-dihydropyrimido[5,4-d]pyrimidin-2-yl)-N-methylmorpholine-2-carboxamide;
8-(4-chloro-2-fluorophenyl)-2,3-dimethyl-6-(2-(5-methyl-1,2,4-oxadiazol-3-yl)morpholino)pyrimido[5,4-d]pyrimidin-4 (3H)-one;
4-(4-(4-chloro-2-fluorophenyl)-6,7-dimethyl-8-oxo-7,8-dihydropyrimido[5,4-d]pyrimidin-2-yl)-N,N-dimethylmorpholine-2-carboxamide;
8-(4-chloro-2-fluorophenyl)-6-(2-(methoxymethyl)morpholino)-2,3-dimethylpyrimido[5,4-d]pyrimidin-4 (3H)-one;
8-(4-chloro-2-fluorophenyl)-6-(2-((dimethylamino)methyl)morpholino)-2,3-dimethylpyrimido[5,4-d]pyrimidin-4 (3H)-one;
8-(4-chloro-2-fluorophenyl)-2,3-dimethyl-6-(6-oxa-9-azaspiro[4.5]decan-9-yl)pyrimido[5,4-d]pyrimidin-4 (3H)-one;
8-(4-chloro-2-fluorophenyl)-2,3-dimethyl-6-(2-methyl-2-(1-methyl-1H-pyrazol-4-yl)morpholino)pyrimido[5,4-d]pyrimidin-4 (3H)-one;
8-(4-chloro-2-fluorophenyl)-2,3-dimethyl-6-(9-methyl-1-oxa-4,9-diazaspiro[5.5]undecan-4-yl)pyrimido[5,4-d]pyrimidin-4 (3H)-one;
8-(4-chloro-2-fluorophenyl)-6-(2,2-dimethylmorpholino)-2,3-dimethylpyrimido[5,4-d]pyrimidin-4 (3H)-one;
6-(2-(1,2,4-oxadiazol-3-yl)morpholino)-8-(4-chloro-2-fluorophenyl)-2,3-dimethylpyrimido[5,4-d]pyrimidin-4 (3H)-one;
8-(4-chloro-2-fluorophenyl)-3-(4-methoxybenzyl)-2-methyl-6-(2-(1-methyl-1H-pyrazol-4-yl)morpholino)pyrimido[5,4-d]pyrimidin-4 (3H)-one;
8-(4-chloro-2-fluorophenyl)-2-methyl-6-(2-(1-methyl-1H-pyrazol-4-yl)morpholino)pyrimido[5,4-d]pyrimidin-4 (3H)-one;
8-(4-chloro-2-fluorophenyl)-3-(2-methoxyethyl)-2-methyl-6-(2-(1-methyl-1H-pyrazol-4-yl)morpholino)pyrimido[5,4-d]pyrimidin-4 (3H)-one;
8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one;
8-(4-chloro-2-fluorophenyl)-2,3-dimethyl-6-[3-(1-methyl-1H-pyrazol-4-yl)pyrrolidin-1-yl]-3H,4H-pyrimido[5,4-d][1,3]diazin-4-one;
(R)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one;
8-(2-fluoro-4-(trifluoromethyl)phenyl)-2,3-dimethyl-6-((2S,4R)-2-(1-methyl-1H-pyrazol-4-yl)tetrahydro-2H-pyran-4-yl)pyrimido[5,4-d]pyrimidin-4 (3H)-one;
8-(2-fluoro-4-(trifluoromethyl)phenyl)-2,3-dimethyl-6-((2R,4S)-2-(1-methyl-1H-pyrazol-4-yl)tetrahydro-2H-pyran-4-yl)pyrimido[5,4-d]pyrimidin-4 (3H)-one;
8-(4-chloro-2-fluorophenyl)-2,3-dimethyl-6-((2S,4R)-2-(1-methyl-1H-pyrazol-4-yl)tetrahydro-2H-pyran-4-yl)pyrimido[5,4-d]pyrimidin-4 (3H)-one;
8-(4-chloro-2-fluorophenyl)-2,3-dimethyl-6-((2R,4S)-2-(1-methyl-1H-pyrazol-4-yl)tetrahydro-2H-pyran-4-yl)pyrimido[5,4-d]pyrimidin-4 (3H)-one;
8-(4-chloro-2-fluorophenyl)-6-(2-(1-(2-methoxyethyl)-1H-pyrazol-4-yl)morpholino)-2,3-dimethylpyrimido[5,4-d]pyrimidin-4 (3H)-one;
8-(2-chloro-4-fluorophenyl)-2,3-dimethyl-6-(2-(1-methyl-1H-pyrazol-4-yl)morpholino)pyrimido[5,4-d]pyrimidin-4 (3H)-one;
2,3-dimethyl-6-((R)-2-(1-methyl-1H-pyrazol-4-yl)morpholino)-8-((1r,4R)-4-methylcyclohexyl)pyrimido[5,4-d]pyrimidin-4 (3H)-one;
2,3-dimethyl-6-((S)-2-(1-methyl-1H-pyrazol-4-yl)morpholino)-8-((1r,4S)-4-methylcyclohexyl)pyrimido[5,4-d]pyrimidin-4 (3H)-one;
8-(6,6-difluorospiro[3.3]heptan-2-yl)-2,3-dimethyl-6-(2-(1-methyl-1H-pyrazol-4-yl)morpholino)pyrimido[5,4-d]pyrimidin-4 (3H)-one;
8-(4-chloro-2-fluorophenyl)-2,3-dimethyl-6-(2-(1-methyl-1H-1,2,3-triazol-4-yl)morpholino)pyrimido[5,4-d]pyrimidin-4 (3H)-one;
(S)-8-(4-chloro-2-fluorophenyl)-2,3-dimethyl-6-(3-((1-methyl-1H-pyrazol-4-yl)oxy)pyrrolidin-1-yl)pyrimido[5,4-d]pyrimidin-4 (3H)-one;
(S)-8-(4-chloro-2-fluorophenyl)-2,3-dimethyl-6-(3-((1-methyl-1H-pyrazol-4-yl)oxy)piperidin-1-yl)pyrimido[5,4-d]pyrimidin-4 (3H)-one;
(R)-8-(4-chloro-2-fluorophenyl)-2,3-dimethyl-6-(3-((1-methyl-1H-pyrazol-4-yl)oxy)piperidin-1-yl)pyrimido[5,4-d]pyrimidin-4 (3H)-one;
8-(4-chloro-2-fluorophenyl)-2,3-dimethyl-6-(4-((1-methyl-1H-pyrazol-4-yl)oxy)piperidin-1-yl)pyrimido[5,4-d]pyrimidin-4 (3H)-one;
8-(4-chloro-2-fluorophenyl)-2,3-dimethyl-6-(3-morpholinopiperidin-1-yl)pyrimido[5,4-d]pyrimidin-4 (3H)-one;
8-(4-chloro-2-fluorophenyl)-6-(3-(3-methoxyazetidin-1-yl)piperidin-1-yl)-2,3-dimethylpyrimido[5,4-d]pyrimidin-4 (3H)-one;
8-(4-chloro-2-fluorophenyl)-6-(3-(3,3-difluoroazetidin-1-yl)piperidin-1-yl)-2,3-dimethylpyrimido[5,4-d]pyrimidin-4 (3H)-one;
6-(2-(1H-imidazol-2-yl)morpholino)-8-(4-chloro-2-fluorophenyl)-2,3-dimethylpyrimido[5,4-d]pyrimidin-4 (3H)-one;
8-(4-chloro-2-fluorophenyl)-2,3-dimethyl-6-(2-(1-methyl-1H-imidazol-4-yl)morpholino)pyrimido[5,4-d]pyrimidin-4 (3H)-one;
8-(4-chloro-2-fluorophenyl)-2,3-dimethyl-6-(5,6,8,9-tetrahydro-7H-pyrimido[4,5-d]azepin-7-yl)pyrimido[5,4-d]pyrimidin-4 (3H)-one;
8-(4-chloro-2-fluorophenyl)-2,3-dimethyl-6-(4-((3-methylpyridin-4-yl)oxy)piperidin-1-yl)pyrimido[5,4-d]pyrimidin-4 (3H)-one;
8-(4-chloro-2-fluorophenyl)-6-(2,3-dihydrospiro[indene-1,2′-morpholin]-4′-yl)-2,3-dimethylpyrimido[5,4-d]pyrimidin-4 (3H)-one;
8-(4-chloro-2-fluorophenyl)-2,3-dimethyl-6-(5,6,7,9-tetrahydro-8H-pyrido[3,4-c]azepin-8-yl)pyrimido[5,4-d]pyrimidin-4 (3H)-one;
2,3-dimethyl-6-(2-(1-methyl-1H-pyrazol-4-yl)morpholino)-8-(piperidin-4-yl)pyrimido[5,4-d]pyrimidin-4 (3H)-one;
2,3-dimethyl-6-((2S,4R)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one;
2,3-dimethyl-6-((2R,4S)-2-(1-methyl-1H-pyrazol-4-yl)tetrahydro-2H-pyran-4-yl)-8-(6-(trifluoromethyl)pyridin-3-yl)pyrimido[5,4-d]pyrimidin-4 (3H)-one;
(R)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one;
(S)-8-(4-chloro-2-fluorophenyl)-6-(2,2-dimethyl-6-(1-methyl-1H-pyrazol-4-yl)morpholino)-2,3-dimethylpyrimido[5,4-d]pyrimidin-4 (3H)-one;
8-(4-chloro-2-fluorophenyl)-2,3-dimethyl-6-(6-(1-methyl-1H-pyrazol-4-yl)-5-oxa-8-azaspiro(3,5)nonan-8-yl)pyrimido(5,4-d)pyrimidin-4 (3H)-one;
8-(4-chloro-2-fluorophenyl)-2,3-dimethyl-6-(2-(2-methyl-2H-1,2,3-triazol-4-yl)morpholino)pyrimido[5,4-d]pyrimidin-4 (3H)-one;
(R)-6-(4,4-difluoro-3-(1-methyl-1H-pyrazol-4-yl)piperidin-1-yl)-2,3-dimethyl-8-(6-(trifluoromethyl)pyridin-3-yl)pyrimido[5,4-d]pyrimidin-4 (3H)-one;
(S)-6-(4,4-difluoro-3-(1-methyl-1H-pyrazol-4-yl)piperidin-1-yl)-2,3-dimethyl-8-(6-(trifluoromethyl)pyridin-3-yl)pyrimido[5,4-d]pyrimidin-4 (3H)-one;
8-(4-chloro-2-fluorophenyl)-2,3-dimethyl-6-((2R,4S)-2-(2-methylpyridin-4-yl)tetrahydro-2H-pyran-4-yl)pyrimido[5,4-d]pyrimidin-4 (3H)-one;
8-(4-chloro-2-fluorophenyl)-2,3-dimethyl-6-((2R,4R)-2-(2-methylpyridin-4-yl)tetrahydro-2H-pyran-4-yl)pyrimido[5,4-d]pyrimidin-4 (3H)-one;
6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylpyrimido[5,4-d]pyrimidin-4 (3H)-one;
6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylpyrimido[5,4-d]pyrimidin-4 (3H)-one;
6-((2S,4R)-2-(1-cyclopropyl-1H-pyrazol-4-yl)tetrahydro-2H-pyran-4-yl)-2,3-dimethyl-8-(2,4,5-trifluorophenyl)pyrimido[5,4-d]pyrimidin-4 (3H)-one;
6-((2R,4S)-2-(1-cyclopropyl-1H-pyrazol-4-yl)tetrahydro-2H-pyran-4-yl)-2,3-dimethyl-8-(2,4,5-trifluorophenyl)pyrimido[5,4-d]pyrimidin-4 (3H)-one;
6-((2S,4R)-2-(1-cyclopropyl-1H-pyrazol-4-yl)tetrahydro-2H-pyran-4-yl)-2,3-dimethyl-8-(6-(trifluoromethyl)pyridin-3-yl)pyrimido[5,4-d]pyrimidin-4 (3H)-one;
6-((2R,4S)-2-(1-cyclopropyl-1H-pyrazol-4-yl)tetrahydro-2H-pyran-4-yl)-2,3-dimethyl-8-(6-(trifluoromethyl)pyridin-3-yl)pyrimido[5,4-d]pyrimidin-4 (3H)-one;
2,3-dimethyl-6-(2-(1-methyl-1H-pyrazol-4-yl)morpholino)-8-(1-methyl-3-(trifluoromethyl)-1H-pyrazol-5-yl)pyrimido[5,4-d]pyrimidin-4 (3H)-one;
(R)-6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one;
(S)-6-(2,2-dimethyl-6-(1-methyl-1H-pyrazol-4-yl)morpholino)-8-(2-fluoro-4-(trifluoro-15-methyl)phenyl)-2,3-dimethylpyrimido[5,4-d]pyrimidin-4 (3H)-one;
(R)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one;
(S)-6-(2,2-dimethyl-6-(1-methyl-1H-pyrazol-4-yl)morpholino)-2,3-dimethyl-8-(2,4,5-trifluorophenyl)pyrimido[5,4-d]pyrimidin-4 (3H)-one;
(R)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one;
(S)-6-(2,2-dimethyl-6-(1-methyl-1H-pyrazol-4-yl)morpholino)-2,3-dimethyl-8-(6-(trifluoromethyl)pyridin-3-yl)pyrimido[5,4-d]pyrimidin-4 (3H)-one;
(S)-8-(4-chloro-2-fluorophenyl)-3-methyl-6-(2-(1-methyl-1H-pyrazol-4-yl)morpholino)-2-(trifluoromethyl)pyrimido[5,4-d]pyrimidin-4 (3H)-one;
8-(4-chloro-2-fluorophenyl)-2,3-dimethyl-6-(2-(1-methyl-1H-pyrazol-4-yl)morpholino)pyrido[3,2-d]pyrimidin-4 (3H)-one;
8-(4-chloro-2-fluorophenyl)-2,3-dimethyl-6-(2-(1-methyl-1H-pyrazol-4-yl)morpholino)pyrido[3,2-d]pyrimidin-4 (3H)-one;
8-(4-chloro-2-fluorophenyl)-2,3-dimethyl-6-(2-(1-methyl-1H-pyrazol-4-yl)morpholino)quinazolin-4 (3H)-one;
(R)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one;
(S)-8-(4-chloro-2-fluorophenyl)-2,3-dimethyl-6-(2-(1-(trifluoromethyl)-1H-pyrazol-4-yl)morpholino)pyrimido[5,4-d]pyrimidin-4 (3H)-one;
8-(4-chloro-2-fluorophenyl)-2,3-dimethyl-6-(5-(1-methyl-1H-pyrazol-4-yl)-4-oxa-7-azaspiro(2,5)octan-7-yl)pyrimido(5,4-d)pyrimidin-4 (3H)-one;
(S)-2,3-dimethyl-6-(2-(1-methyl-1H-pyrazol-4-yl)morpholino)-8-(3-(trifluoromethyl)bicyclo[1.1.1]pentan-1-yl)pyrimido[5,4-d]pyrimidin-4 (3H)-one;
4-(4-chloro-2-fluorophenyl)-6,7-dimethyl-2-(2-(1-methyl-1H-pyrazol-4-yl)morpholino)pyrido[3,4-d]pyrimidin-8 (7H)-one;
8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one;
8-(4-chloro-2-fluorophenyl)-6-(2-(1-cyclopropyl-1H-pyrazol-4-yl)-3-oxabicyclo[4.1.0]heptan-6-yl)-2,3-dimethylpyrimido[5,4-d]pyrimidin-4 (3H)-one;
8-(4-chloro-2-fluorophenyl)-6-((1S,2S,6R)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one;
8-(4-chloro-2-fluorophenyl)-6-((1R,2R,6S)-2-(1-cyclopropyl-1H-pyrazol-4-yl)-3-oxabicyclo[4.1.0]heptan-6-yl)-2,3-dimethylpyrimido[5,4-d]pyrimidin-4 (3H)-one;
(R)-8-(4-chloro-2-fluorophenyl)-6-(2,2-dimethyl-6-(1-methyl-1H-pyrazol-4-yl)morpholino)-2,3-dimethylquinazolin-4 (3H)-one;
(S)-8-(4-chloro-2-fluorophenyl)-6-(2,2-dimethyl-6-(1-methyl-1H-pyrazol-4-yl)morpholino)-2,3-dimethylquinazolin-4 (3H)-one;
6-(2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one;
6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one;
6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one;
8-(4-chloro-2-fluorophenyl)-6-((2S,4R)-2-(1-cyclopropyl-1H-pyrazol-4-yl)tetrahydro-2H-pyran-4-yl)-2,3-dimethylquinazolin-4 (3H)-one;
8-(4-chloro-2-fluorophenyl)-6-((2R,4S)-2-(1-cyclopropyl-1H-pyrazol-4-yl)tetrahydro-2H-pyran-4-yl)-2,3-dimethylquinazolin-4 (3H)-one;
6-((2S,4R)-2-(1-cyclopropyl-1H-pyrazol-4-yl)tetrahydro-2H-pyran-4-yl)-8-(2,4-difluorophenyl)-2,3-dimethylpyrimido[5,4-d]pyrimidin-4 (3H)-one;
6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylquinazolin-4 (3H)-one;
8-(2,4-difluorophenyl)-6-(2,2-dimethyl-6-(1-methyl-1H-pyrazol-4-yl)morpholino)-2,3-dimethylpyrimido[5,4-d]pyrimidin-4 (3H)-one;
(R)-8-(2,4-difluorophenyl)-6-(2,2-dimethyl-6-(1-methyl-1H-pyrazol-4-yl)morpholino)-2,3-dimethylpyrimido[5,4-d]pyrimidin-4 (3H)-one;
(S)-8-(2,4-difluorophenyl)-6-(2,2-dimethyl-6-(1-methyl-1H-pyrazol-4-yl)morpholino)-2,3-dimethylpyrimido[5,4-d]pyrimidin-4 (3H)-one;
8-(2,4-difluorophenyl)-2,3-dimethyl-6-(2-(2-methylpyridin-4-yl)morpholino)pyrimido[5,4-d]pyrimidin-4 (3H)-one;
(R)-8-(2,4-difluorophenyl)-2,3-dimethyl-6-(2-(2-methylpyridin-4-yl)morpholino)pyrimido[5,4-d]pyrimidin-4 (3H)-one;
(S)-8-(2,4-difluorophenyl)-2,3-dimethyl-6-(2-(2-methylpyridin-4-yl)morpholino)pyrimido[5,4-d]pyrimidin-4 (3H)-one;
2,3-dimethyl-6-(2-(2-methylpyridin-4-yl)morpholino)-8-(2,4,5-trifluorophenyl)pyrimido[5,4-d]pyrimidin-4 (3H)-one;
(R)-2,3-dimethyl-6-(2-(2-methylpyridin-4-yl)morpholino)-8-(2,4,5-trifluorophenyl)pyrimido[5,4-d]pyrimidin-4 (3H)-one
(S)-2,3-dimethyl-6-(2-(2-methylpyridin-4-yl)morpholino)-8-(2,4,5-trifluorophenyl)pyrimido[5,4-d]pyrimidin-4 (3H)-one;
6-(2,2-dimethyl-6-(2-methylpyridin-4-yl)morpholino)-8-(2-fluoro-4-(trifluoromethyl)phenyl)-2,3-dimethylpyrimido[5,4-d]pyrimidin-4 (3H)-one;
(R)-6-(2,2-dimethyl-6-(2-methylpyridin-4-yl)morpholino)-8-(2-fluoro-4-(trifluoromethyl)phenyl)-2,3-dimethylpyrimido[5,4-d]pyrimidin-4 (3H)-one;
(S)-6-(2,2-dimethyl-6-(2-methylpyridin-4-yl)morpholino)-8-(2-fluoro-4-(trifluoromethyl)phenyl)-2,3-dimethylpyrimido[5,4-d]pyrimidin-4 (3H)-one;
8-(4-chloro-2-fluorophenyl)-2,3-dimethyl-6-(2-(1-methyl-1H-pyrazol-4-yl)morpholino)pyrido[3,4-d]pyrimidin-4 (3H)-one;
(S)-8-(4-chloro-2-fluorophenyl)-2,3-dimethyl-6-(2-(1-methyl-1H-pyrazol-4-yl)morpholino)pyrido[3,4-d]pyrimidin-4 (3H)-one;
(R)-8-(4-chloro-2-fluorophenyl)-2,3-dimethyl-6-(2-(1-methyl-1H-pyrazol-4-yl)morpholino)pyrido[3,4-d]pyrimidin-4 (3H)-one;
8-(2-fluoro-4-(trifluoromethyl)phenyl)-2,3-dimethyl-6-(2-(1-methyl-1H-pyrazol-4-yl)morpholino)pyrido[3,4-d]pyrimidin-4 (3H)-one;
(S)-8-(2-fluoro-4-(trifluoromethyl)phenyl)-2,3-dimethyl-6-(2-(1-methyl-1H-pyrazol-4-yl)morpholino)pyrido[3,4-d]pyrimidin-4 (3H)-one;
(R)-8-(2-fluoro-4-(trifluoromethyl)phenyl)-2,3-dimethyl-6-(2-(1-methyl-1H-pyrazol-4-yl)morpholino)pyrido[3,4-d]pyrimidin-4 (3H)-one;
2,3-dimethyl-6-(2-(1-methyl-1H-pyrazol-4-yl)morpholino)-8-(6-(trifluoromethyl)pyridin-3-yl)pyrido[3,4-d]pyrimidin-4 (3H)-one;
(S)-2,3-dimethyl-6-(2-(1-methyl-1H-pyrazol-4-yl)morpholino)-8-(6-(trifluoromethyl)pyridin-3-yl)pyrido[3,4-d]pyrimidin-4 (3H)-one;
(R)-2,3-dimethyl-6-(2-(1-methyl-1H-pyrazol-4-yl)morpholino)-8-(6-(trifluoromethyl)pyridin-3-yl)pyrido[3,4-d]pyrimidin-4 (3H)-one;
(R)-8-(4-chloro-2-fluorophenyl)-2,3-dimethyl-6-(5-(2-methylpyridin-4-yl)-4-oxa-7-azaspiro[2.5]octan-7-yl)pyrido[3,4-d]pyrimidin-4 (3H)-one;
(S)-8-(4-chloro-2-fluorophenyl)-2,3-dimethyl-6-(5-(2-methylpyridin-4-yl)-4-oxa-7-azaspiro[2.5]octan-7-yl)pyrido[3,4-d]pyrimidin-4 (3H)-one;
(S)-8-(2,4-difluorophenyl)-2,3-dimethyl-6-(2-(1-methyl-1H-pyrazol-4-yl)morpholino)pyrido[3,4-d]pyrimidin-4 (3H)-one;
(R)-8-(2,4-difluorophenyl)-2,3-dimethyl-6-(2-(1-methyl-1H-pyrazol-4-yl)morpholino)pyrido[3,4-d]pyrimidin-4 (3H)-one;
(S)-2,3-dimethyl-6-(2-(1-methyl-1H-pyrazol-4-yl)morpholino)-8-(2,4,5-trifluorophenyl)pyrido[3,4-d]pyrimidin-4 (3H)-one;
(R)-2,3-dimethyl-6-(2-(1-methyl-1H-pyrazol-4-yl)morpholino)-8-(2,4,5-trifluorophenyl)pyrido[3,4-d]pyrimidin-4 (3H)-one;
(R)-8-(4-chloro-2-fluorophenyl)-6-(2-(1-cyclopropyl-1H-pyrazol-4-yl)morpholino)-2,3-dimethylpyrido[3,4-d]pyrimidin-4 (3H)-one;
(S)-8-(4-chloro-2-fluorophenyl)-6-(2-(1-cyclopropyl-1H-pyrazol-4-yl)morpholino)-2,3-dimethylpyrido[3,4-d]pyrimidin-4 (3H)-one;
(R)-6-(2-(1-cyclopropyl-1H-pyrazol-4-yl)morpholino)-8-(2-fluoro-4-(trifluoromethyl)phenyl)-2,3-dimethylpyrido[3,4-d]pyrimidin-4 (3H)-one;
(S)-6-(2-(1-cyclopropyl-1H-pyrazol-4-yl)morpholino)-8-(2-fluoro-4-(trifluoromethyl)phenyl)-2,3-dimethylpyrido[3,4-d]pyrimidin-4 (3H)-one;
(R)-6-(2-(1-cyclopropyl-1H-pyrazol-4-yl)morpholino)-2,3-dimethyl-8-(2,4,5-trifluorophenyl)pyrido[3,4-d]pyrimidin-4 (3H)-one;
(S)-6-(2-(1-cyclopropyl-1H-pyrazol-4-yl)morpholino)-2,3-dimethyl-8-(2,4,5-trifluorophenyl)pyrido[3,4-d]pyrimidin-4 (3H)-one;
(R)-6-(2,2-dimethyl-6-(1-methyl-1H-pyrazol-4-yl)morpholino)-8-(2-fluoro-4-(trifluoromethyl)phenyl)-2,3-dimethylpyrido[3,4-d]pyrimidin-4 (3H)-one;
(S)-6-(2,2-dimethyl-6-(1-methyl-1H-pyrazol-4-yl)morpholino)-8-(2-fluoro-4-(trifluoromethyl)phenyl)-2,3-dimethylpyrido[3,4-d]pyrimidin-4 (3H)-one;
(R)-8-(4-chloro-2-fluorophenyl)-2,3-dimethyl-6-(2-(2-methylpyridin-4-yl)morpholino)pyrido[3,4-d]pyrimidin-4 (3H)-one;
(S)-8-(4-chloro-2-fluorophenyl)-2,3-dimethyl-6-(2-(2-methylpyridin-4-yl)morpholino)pyrido[3,4-d]pyrimidin-4 (3H)-one;
(R)-8-(2,4-difluorophenyl)-2,3-dimethyl-6-(2-(2-methylpyridin-4-yl)morpholino)pyrido[3,4-d]pyrimidin-4 (3H)-one;
(S)-8-(2,4-difluorophenyl)-2,3-dimethyl-6-(2-(2-methylpyridin-4-yl)morpholino)pyrido[3,4-d]pyrimidin-4 (3H)-one;
(R)-8-(2-fluoro-4-(trifluoromethyl)phenyl)-2,3-dimethyl-6-(2-(2-methylpyridin-4-yl)morpholino)pyrido[3,4-d]pyrimidin-4 (3H)-one;
(S)-8-(2-fluoro-4-(trifluoromethyl)phenyl)-2,3-dimethyl-6-(2-(2-methylpyridin-4-yl)morpholino)pyrido[3,4-d]pyrimidin-4 (3H)-one;
(R)-2,3-dimethyl-6-(2-(2-methylpyridin-4-yl)morpholino)-8-(6-(trifluoromethyl)pyridin-3-yl)pyrido[3,4-d]pyrimidin-4 (3H)-one;
(S)-2,3-dimethyl-6-(2-(2-methylpyridin-4-yl)morpholino)-8-(6-(trifluoromethyl)pyridin-3-yl)pyrido[3,4-d]pyrimidin-4 (3H)-one;
6-((2S,4R)-2-(1-cyclopropyl-1H-pyrazol-4-yl)tetrahydro-2H-pyran-4-yl)-2,3-dimethyl-8-(2,4,5-trifluorophenyl)pyrido[3,4-d]pyrimidin-4 (3H)-one;
6-((2R,4S)-2-(1-cyclopropyl-1H-pyrazol-4-yl)tetrahydro-2H-pyran-4-yl)-2,3-dimethyl-8-(2,4,5-trifluorophenyl)pyrido[3,4-d]pyrimidin-4 (3H)-one;
8-(4-chloro-2-fluorophenyl)-6-((2S,4R)-2-(1-cyclopropyl-1H-pyrazol-4-yl)tetrahydro-2H-pyran-4-yl)-2,3-dimethylpyrido[3,4-d]pyrimidin-4 (3H)-one;
8-(4-chloro-2-fluorophenyl)-6-((2R,4S)-2-(1-cyclopropyl-1H-pyrazol-4-yl)tetrahydro-2H-pyran-4-yl)-2,3-dimethylpyrido[3,4-d]pyrimidin-4 (3H)-one;
(R)-2,3-dimethyl-6-(2-(2-methylpyridin-4-yl)morpholino)-8-(2,4,5-trifluorophenyl)pyrido[3,4-d]pyrimidin-4 (3H)-one;
(S)-2,3-dimethyl-6-(2-(2-methylpyridin-4-yl)morpholino)-8-(2,4,5-trifluorophenyl)pyrido[3,4-d]pyrimidin-4 (3H)-one;
(R)8-(4-chloro-2-fluorophenyl)-6-(2,2-dimethyl-6-(1-methyl-1H-pyrazol-4-yl)morpholino)-2,3-dimethylpyrido[3,2-d]pyrimidin-4 (3H)-one;
(S)8-(4-chloro-2-fluorophenyl)-6-(2,2-dimethyl-6-(1-methyl-1H-pyrazol-4-yl)morpholino)-2,3-dimethylpyrido[3,2-d]pyrimidin-4 (3H)-one;
(R)-8-(2-fluoro-4-(trifluoromethyl)phenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one;
(S)-8-(2-fluoro-4-(trifluoromethyl)phenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one;
(R)-8-(4-chloro-2-fluorophenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one;
(S)-8-(4-chloro-2-fluorophenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one;
(R)-8-(2-fluoro-4-(trifluoromethyl)phenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one;
(S)-8-(2-fluoro-4-(trifluoromethyl)phenyl)-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one;
(R)-2,3-dimethyl-6-(5-(1-methyl-1H-pyrazol-4-yl)-4-oxa-7-azaspiro[2.5]octan-7-yl)-8-(2,4,5-trifluorophenyl)pyrido[3,4-d]pyrimidin-4 (3H)-one;
(S)-2,3-dimethyl-6-(5-(1-methyl-1H-pyrazol-4-yl)-4-oxa-7-azaspiro[2.5]octan-7-yl)-8-(2,4,5-trifluorophenyl)pyrido[3,4-d]pyrimidin-4 (3H)-one;
(S)-8-(4-chloro-2-fluorophenyl)-6-(2,2-dimethyl-6-(2-methyl-2H-1,2,3-triazol-4-yl)morpholino)-2,3-dimethylpyrido[3,4-d]pyrimidin-4 (3H)-one;
(R)-8-(4-chloro-2-fluorophenyl)-6-(2,2-dimethyl-6-(2-methyl-2H-1,2,3-triazol-4-yl)morpholino)-2,3-dimethylpyrido[3,4-d]pyrimidin-4 (3H)-one;
8-(4-chloro-2-fluorophenyl)-2,3-dimethyl-6-((1R,2S)-2-(1-methyl-1H-pyrazol-4-yl)cyclopropyl)pyrido[3,4-d]pyrimidin-4 (3H)-one;
8-(4-chloro-2-fluorophenyl)-2,3-dimethyl-6-((1R,2R)-2-(1-methyl-1H-pyrazol-4-yl)cyclopropyl)pyrido[3,4-d]pyrimidin-4 (3H)-one;
(R)-8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one;
(S)-8-(4-chloro-2-fluorophenyl)-6-(2,2-difluoro-6-(1-methyl-1H-pyrazol-4-yl)morpholino)-2,3-dimethylpyrimido[5,4-d]pyrimidin-4 (3H)-one;
(R)-6-(2,2-dimethyl-6-(1-methyl-1H-pyrazol-4-yl)morpholino)-2,3-dimethyl-8-(2,4,5-trifluorophenyl)pyrido[3,4-d]pyrimidin-4 (3H)-one;
(S)-6-(2,2-dimethyl-6-(1-methyl-1H-pyrazol-4-yl)morpholino)-2,3-dimethyl-8-(2,4,5-trifluorophenyl)pyrido[3,4-d]pyrimidin-4 (3H)-one;
(R)-8-(4-chloro-2-fluorophenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one;
(S)-8-(4-chloro-2-fluorophenyl)-2,3-dimethyl-6-(6-(1-methyl-1H-pyrazol-4-yl)-5-oxa-8-azaspiro[3.5]nonan-8-yl)pyrido[3,4-d]pyrimidin-4 (3H)-one;
(R)-2,3-dimethyl-6-(6-(1-methyl-1H-pyrazol-4-yl)-5-oxa-8-azaspiro[3.5]nonan-8-yl)-8-(2,4,5-trifluorophenyl)pyrido[3,4-d]pyrimidin-4 (3H)-one;
(S)-2,3-dimethyl-6-(6-(1-methyl-1H-pyrazol-4-yl)-5-oxa-8-azaspiro[3.5]nonan-8-yl)-8-(2,4,5-trifluorophenyl)pyrido[3,4-d]pyrimidin-4 (3H)-one;
6-((2S,4R)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one;
6-((2R,4S)-2-(1-cyclopropyl-1H-pyrazol-4-yl)tetrahydro-2H-pyran-4-yl)-8-(2-fluoro-4-(trifluoromethyl)phenyl)-2,3-dimethylpyrido[3,4-d]pyrimidin-4 (3H)-one;
(R)-8-(2,4-difluorophenyl)-3-methyl-6-(2-(2-methylpyridin-4-yl)morpholino)-2-(trifluoromethyl)pyrimido[5,4-d]pyrimidin-4 (3H)-one;
(S)-8-(2,4-difluorophenyl)-3-methyl-6-(2-(2-methylpyridin-4-yl)morpholino)-2-(trifluoromethyl)pyrimido[5,4-d]pyrimidin-4 (3H)-one;
(R)-3-methyl-6-(2-(2-methylpyridin-4-yl)morpholino)-2-(trifluoromethyl)-8-(2,4,5-trifluorophenyl)pyrimido[5,4-d]pyrimidin-4 (3H)-one;
(S)-3-methyl-6-(2-(2-methylpyridin-4-yl)morpholino)-2-(trifluoromethyl)-8-(2,4,5-trifluorophenyl)pyrimido[5,4-d]pyrimidin-4 (3H)-one;
(R)-6-(2,2-difluoro-6-(1-methyl-1H-pyrazol-4-yl)morpholino)-8-(2,4-difluorophenyl)-2,3-dimethylpyrimido[5,4-d]pyrimidin-4 (3H)-one;
(S)-6-(2,2-difluoro-6-(1-methyl-1H-pyrazol-4-yl)morpholino)-8-(2,4-difluorophenyl)-2,3-dimethylpyrimido[5,4-d]pyrimidin-4 (3H)-one;
8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one;
8-(2,4-difluorophenyl)-5-fluoro-3-methyl-6-(5-(1-methyl-1H-pyrazol-4-yl)-4-oxa-7-azaspiro[2.5]octan-7-yl)-2-(trifluoromethyl)pyrido[3,4-d]pyrimidin-4 (3H)-one;
(R)-8-(2,4-difluorophenyl)-6-(2,2-dimethyl-6-(2-methylpyridin-4-yl)morpholino)-2,3-dimethylpyrido[3,4-d]pyrimidin-4 (3H)-one;
(S)-8-(2,4-difluorophenyl)-6-(2,2-dimethyl-6-(2-methylpyridin-4-yl)morpholino)-2,3-dimethylpyrido[3,4-d]pyrimidin-4 (3H)-one;
(R)-6-(2,2-dimethyl-6-(2-methylpyridin-4-yl)morpholino)-2,3-dimethyl-8-(2,4,5-trifluorophenyl)pyrido[3,4-d]pyrimidin-4 (3H)-one;
(S)-6-(2,2-dimethyl-6-(2-methylpyridin-4-yl)morpholino)-2,3-dimethyl-8-(2,4,5-trifluorophenyl)pyrido[3,4-d]pyrimidin-4 (3H)-one;
(R)-6-(2,2-dimethyl-6-(2-methylpyridin-4-yl)morpholino)-2,3-dimethyl-8-(6-(trifluoromethyl)pyridin-3-yl)pyrido[3,4-d]pyrimidin-4 (3H)-one;
(S)-6-(2,2-dimethyl-6-(2-methylpyridin-4-yl)morpholino)-2,3-dimethyl-8-(6-(trifluoromethyl)pyridin-3-yl)pyrido[3,4-d]pyrimidin-4 (3H)-one;
(R)-3-methyl-6-(5-(1-methyl-1H-pyrazol-4-yl)-4-oxa-7-azaspiro[2.5]octan-7-yl)-2-(trifluoromethyl)-8-(2,4,5-trifluorophenyl)pyrimido[5,4-d]pyrimidin-4 (3H)-one;
(S)-3-methyl-6-(5-(1-methyl-1H-pyrazol-4-yl)-4-oxa-7-azaspiro[2.5]octan-7-yl)-2-(trifluoromethyl)-8-(2,4,5-trifluorophenyl)pyrimido[5,4-d]pyrimidin-4 (3H)-one;
(R)-6-(2,2-dimethyl-6-(2-methylpyridin-4-yl)morpholino)-8-(2-fluoro-4-(trifluoromethyl)phenyl)-2,3-dimethylpyrido[3,4-d]pyrimidin-4 (3H)-one;
(S)-6-(2,2-dimethyl-6-(2-methylpyridin-4-yl)morpholino)-8-(2-fluoro-4-(trifluoromethyl)phenyl)-2,3-dimethylpyrido[3,4-d]pyrimidin-4 (3H)-one;
(R)-6-(2,2-dimethyl-6-(2-methylpyridin-4-yl)morpholino)-2,3-dimethyl-8-(6-(trifluoromethyl)pyridin-3-yl)pyrimido[5,4-d]pyrimidin-4 (3H)-one;
(S)-6-(2,2-dimethyl-6-(2-methylpyridin-4-yl)morpholino)-2,3-dimethyl-8-(6-(trifluoromethyl)pyridin-3-yl)pyrimido[5,4-d]pyrimidin-4 (3H)-one;
(R)-8-(2,4-difluorophenyl)-3-methyl-6-(5-(1-methyl-1H-pyrazol-4-yl)-4-oxa-7-azaspiro[2.5]octan-7-yl)-2-(trifluoromethyl)pyrimido[5,4-d]pyrimidin-4 (3H)-one;
(S)-8-(2,4-difluorophenyl)-3-methyl-6-(5-(1-methyl-1H-pyrazol-4-yl)-4-oxa-7-azaspiro[2.5]octan-7-yl)-2-(trifluoromethyl)pyrimido[5,4-d]pyrimidin-4 (3H)-one;
(R)-6-(2,2-difluoro-6-(1-methyl-1H-pyrazol-4-yl)morpholino)-8-(2,4-difluorophenyl)-2,3-dimethylpyrido[3,4-d]pyrimidin-4 (3H)-one;
(S)-6-(2,2-difluoro-6-(1-methyl-1H-pyrazol-4-yl)morpholino)-8-(2,4-difluorophenyl)-2,3-dimethylpyrido[3,4-d]pyrimidin-4 (3H)-one;
6-(2-(1-cyclopropyl-1H-pyrazol-4-yl)tetrahydro-2H-pyran-4-yl)-8-(2,4-difluorophenyl)-5-fluoro-3-methyl-2-(trifluoromethyl)pyrido[3,4-d]pyrimidin-4 (3H)-one;
8-(2-fluoro-4-(trifluoromethyl)phenyl)-2,3-dimethyl-6-((2S,4R)-2-(2-methylpyridin-4-yl)tetrahydro-2H-pyran-4-yl)pyrido[3,4-d]pyrimidin-4 (3H)-one;
8-(2-fluoro-4-(trifluoromethyl)phenyl)-2,3-dimethyl-6-((2R,4S)-2-(2-methylpyridin-4-yl)tetrahydro-2H-pyran-4-yl)pyrido[3,4-d]pyrimidin-4 (3H)-one;
(R)-6-(2,2-difluoro-6-(2-methylpyridin-4-yl)morpholino)-8-(2,4-difluorophenyl)-2,3-dimethylpyrimido[5,4-d]pyrimidin-4 (3H)-one;
(S)-6-(2,2-difluoro-6-(2-methylpyridin-4-yl)morpholino)-8-(2,4-difluorophenyl)-2,3-dimethylpyrimido[5,4-d]pyrimidin-4 (3H)-one;
(R)-8-(2,4-difluorophenyl)-6-(2,2-dimethyl-6-(2-methylpyridin-4-yl)morpholino)-2,3-dimethylpyrimido[5,4-d]pyrimidin-4 (3H)-one;
(S)-8-(2,4-difluorophenyl)-6-(2,2-dimethyl-6-(2-methylpyridin-4-yl)morpholino)-2,3-dimethylpyrimido[5,4-d]pyrimidin-4 (3H)-one;
(R)-6-(2,2-difluoro-6-(2-methylpyridin-4-yl)morpholino)-8-(2,4-difluorophenyl)-2,3-dimethylpyrido[3,4-d]pyrimidin-4 (3H)-one;
(S)-6-(2,2-difluoro-6-(2-methylpyridin-4-yl)morpholino)-8-(2,4-difluorophenyl)-2,3-dimethylpyrido[3,4-d]pyrimidin-4 (3H)-one;
(R)-8-(4-chloro-2-fluorophenyl)-6-(2,2-dimethyl-6-(2-methylpyridin-4-yl)morpholino)-2,3-dimethylpyrido[3,4-d]pyrimidin-4 (3H)-one;
(S)-8-(4-chloro-2-fluorophenyl)-6-(2,2-dimethyl-6-(2-methylpyridin-4-yl)morpholino)-2,3-dimethylpyrido[3,4-d]pyrimidin-4 (3H)-one;
(R)-8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one;
(S)-8-(4-chloro-2-fluorophenyl)-5-fluoro-2,3-dimethyl-6-(5-(1-methyl-1H-pyrazol-4-yl)-4-oxa-7-azaspiro[2.5]octan-7-yl)pyrido[3,4-d]pyrimidin-4 (3H)-one;
(R)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one;
(S)-8-(2,4-difluorophenyl)-5-fluoro-3-methyl-6-(2-(2-methylpyridin-4-yl)morpholino)-2-(trifluoromethyl)pyrido[3,4-d]pyrimidin-4 (3H)-one;
8-(2,4-difluorophenyl)-2,3-dimethyl-6-((2S,6R)-2-methyl-6-(2-methylpyridin-4-yl)morpholino)pyrimido[5,4-d]pyrimidin-4 (3H)-one;
(8-(2,4-difluorophenyl)-2,3-dimethyl-6-((2R,6S)-2-methyl-6-(2-methylpyridin-4-yl)morpholino)pyrimido[5,4-d]pyrimidin-4 (3H)-one;
8-(4-chloro-2-fluorophenyl)-2,3-dimethyl-6-((2S,6R)-2-methyl-6-(2-methylpyridin-4-yl)morpholino)pyrimido[5,4-d]pyrimidin-4 (3H)-one;
8-(4-chloro-2-fluorophenyl)-2,3-dimethyl-6-((2R,6S)-2-methyl-6-(2-methylpyridin-4-yl)morpholino)pyrimido[5,4-d]pyrimidin-4 (3H)-one;
6-((2R,6S)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one;
6-((2S,6R)-2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one;
6-(2-(1-cyclopropyl-1H-pyrazol-4-yl)-6-methylmorpholino)-8-(2,4-difluorophenyl)-2,3-dimethylpyrimido[5,4-d]pyrimidin-4 (3H)-one (trans-racemate);
8-(4-chloro-2-fluorophenyl)-6-((2R,6S)-2-(1-cyclopropyl-1H-pyrazol-4-yl)-6-methylmorpholino)-2,3-dimethylpyrimido[5,4-d]pyrimidin-4 (3H)-one;
8-(4-chloro-2-fluorophenyl)-6-((2S,6R)-2-(1-cyclopropyl-1H-pyrazol-4-yl)-6-methylmorpholino)-2,3-dimethylpyrimido[5,4-d]pyrimidin-4 (3H)-one;
8-(2-fluoro-4-(trifluoromethyl)phenyl)-2,3-dimethyl-6-((2S,6R)-2-methyl-6-(2-methylpyridin-4-yl)morpholino)pyrimido[5,4-d]pyrimidin-4 (3H)-one;
8-(2-fluoro-4-(trifluoromethyl)phenyl)-2,3-dimethyl-6-((2R,6S)-2-methyl-6-(2-methylpyridin-4-yl)morpholino)pyrimido[5,4-d]pyrimidin-4 (3H)-one;
(R)-8-(6-(difluoromethyl)pyridin-3-yl)-6-(2,2-dimethyl-6-(2-methylpyridin-4-yl)morpholino)-2,3-dimethylpyrido[3,4-d]pyrimidin-4 (3H)-one;
(S)-8-(6-(difluoromethyl)pyridin-3-yl)-6-(2,2-dimethyl-6-(2-methylpyridin-4-yl)morpholino)-2,3-dimethylpyrido[3,4-d]pyrimidin-4 (3H)-one;
(R)-6-(2,2-difluoro-6-(2-methylpyridin-4-yl)morpholino)-8-(6-(difluoromethyl)pyridin-3-yl)-2,3-dimethylpyrido[3,4-d]pyrimidin-4 (3H)-one;
(S)-6-(2,2-difluoro-6-(2-methylpyridin-4-yl)morpholino)-8-(6-(difluoromethyl)pyridin-3-yl)-2,3-dimethylpyrido[3,4-d]pyrimidin-4 (3H)-one;
8-(2,4-difluorophenyl)-2,3-dimethyl-6-((2S,6R)-2-methyl-6-(2-methylpyridin-4-yl)morpholino)pyrido[3,4-d]pyrimidin-4 (3H)-one;
8-(2,4-difluorophenyl)-2,3-dimethyl-6-((2R,6S)-2-methyl-6-(2-methylpyridin-4-yl)morpholino)pyrido[3,4-d]pyrimidin-4 (3H)-one;
6-((2R,6S)-2-(1-cyclopropyl-1H-pyrazol-4-yl)-6-methylmorpholino)-8-(2,4-difluorophenyl)-3-methyl-2-(trifluoromethyl)pyrimido[5,4-d]pyrimidin-4 (3H)-one;
6-((2S,6R)-2-(1-cyclopropyl-1H-pyrazol-4-yl)-6-methylmorpholino)-8-(2,4-difluorophenyl)-3-methyl-2-(trifluoromethyl)pyrimido[5,4-d]pyrimidin-4 (3H)-one;
6-((2R,6S)-2-(1-cyclopropyl-1H-pyrazol-4-yl)-6-methylmorpholino)-2,3-dimethyl-8-(6-(trifluoromethyl)pyridin-3-yl)pyrido[3,4-d]pyrimidin-4 (3H)-one;
6-((2S,6R)-2-(1-cyclopropyl-1H-pyrazol-4-yl)-6-methylmorpholino)-2,3-dimethyl-8-(6-(trifluoromethyl)pyridin-3-yl)pyrido[3,4-d]pyrimidin-4 (3H)-one;
(8-(2,4-difluorophenyl)-2,3-dimethyl-6-((2R,4S,6R)-2-methyl-6-(2-methylpyridin-4-yl)tetrahydro-2H-pyran-4-yl)pyrido[3,4-d]pyrimidin-4 (3H)-one;
6-((2S,4R)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one;
6-((2R,4S)-2-(1-cyclopropyl-1H-pyrazol-4-yl)tetrahydro-2H-pyran-4-yl)-3-methyl-2-(trifluoromethyl)-8-(2,4,5-trifluorophenyl)pyrimido[5,4-d]pyrimidin-4 (3H)-one;
8-(4-chloro-2-fluorophenyl)-3-methyl-6-(2-(1-methyl-1H-pyrazol-4-yl)morpholino)pyrimido[5,4-d]pyrimidin-4 (3H)-one;
8-(4-chloro-2-fluorophenyl)-2,3-dimethyl-6-(2-(trifluoromethyl)-5,6-dihydro-[1,2,4]triazolo[1,5-a]pyrazin-7 (8H)-yl)pyrimido[5,4-d]pyrimidin-4 (3H)-one;
8-(2,4-difluorophenyl)-3-methyl-6-((2S,6R)-2-methyl-6-(2-methylpyridin-4-yl)morpholino)-2-(trifluoromethyl)pyrido[3,4-d]pyrimidin-4 (3H)-one;
8-(2,4-difluorophenyl)-3-methyl-6-((2R,6S)-2-methyl-6-(2-methylpyridin-4-yl)morpholino)-2-(trifluoromethyl)pyrido[3,4-d]pyrimidin-4 (3H)-one;
6-((2R,6S)-2-(1-cyclopropyl-1H-pyrazol-4-yl)-6-methylmorpholino)-8-(2,4-difluorophenyl)-2,3-dimethylpyrido[3,4-d]pyrimidin-4 (3H)-one;
6-((2S,6R)-2-(1-cyclopropyl-1H-pyrazol-4-yl)-6-methylmorpholino)-8-(2,4-difluorophenyl)-2,3-dimethylpyrido[3,4-d]pyrimidin-4 (3H)-one;
8-(2,4-difluorophenyl)-3-methyl-6-((2S,6R)-2-methyl-6-(2-methylpyridin-4-yl)morpholino)-2-(trifluoromethyl)pyrimido[5,4-d]pyrimidin-4 (3H)-one;
8-(6-(difluoromethyl)pyridin-3-yl)-2,3-dimethyl-6-((2S,6R)-2-methyl-6-(2-methylpyridin-4-yl)morpholino)pyrido[3,4-d]pyrimidin-4 (3H)-one;
8-(6-(difluoromethyl)pyridin-3-yl)-2,3-dimethyl-6-((2R,6S)-2-methyl-6-(2-methylpyridin-4-yl)morpholino)pyrido[3,4-d]pyrimidin-4 (3H)-one;
6-((2R,6S)-2-(1-cyclopropyl-1H-pyrazol-4-yl)-6-methylmorpholino)-8-(2,4-difluorophenyl)-3-methyl-2-(trifluoromethyl)pyrido[3,4-d]pyrimidin-4 (3H)-one;
6-((2S,6R)-2-(1-cyclopropyl-1H-pyrazol-4-yl)-6-methylmorpholino)-8-(2,4-difluorophenyl)-3-methyl-2-(trifluoromethyl)pyrido[3,4-d]pyrimidin-4 (3H)-one;
6-((2R,6S)-2-(1-cyclopropyl-1H-pyrazol-4-yl)-6-methylmorpholino)-8-(6-(difluoromethyl)pyridin-3-yl)-2,3-dimethylpyrido[3,4-d]pyrimidin-4 (3H)-one;
6-((2S,6R)-2-(1-cyclopropyl-1H-pyrazol-4-yl)-6-methylmorpholino)-8-(6-(difluoromethyl)pyridin-3-yl)-2,3-dimethylpyrido[3,4-d]pyrimidin-4 (3H)-one;
6-((2R,6S)-2-(1-cyclopropyl-1H-pyrazol-4-yl)-6-methylmorpholino)-8-(2-fluoro-4-(trifluoromethyl)phenyl)-2,3-dimethylpyrimido[5,4-d]pyrimidin-4 (3H)-one;
6-((2S,6R)-2-(1-cyclopropyl-1H-pyrazol-4-yl)-6-methylmorpholino)-8-(2-fluoro-4-(trifluoromethyl)phenyl)-2,3-dimethylpyrimido[5,4-d]pyrimidin-4 (3H)-one;
6-((2S,4R)-2-(1-cyclopropyl-1H-pyrazol-4-yl)tetrahydro-2H-pyran-4-yl)-8-(2,4-difluorophenyl)-3-methyl-2-(trifluoromethyl)pyrimido[5,4-d]pyrimidin-4 (3H)-one;
6-((2R,4S)-2-(1-cyclopropyl-1H-pyrazol-4-yl)tetrahydro-2H-pyran-4-yl)-8-(2,4-difluorophenyl)-3-methyl-2-(trifluoromethyl)pyrimido[5,4-d]pyrimidin-4 (3H)-one;
6-((2S,4R)-2-(1-cyclopropyl-1H-pyrazol-4-yl)tetrahydro-2H-pyran-4-yl)-8-(2,4-difluorophenyl)-3-methyl-2-(trifluoromethyl)pyrido[3,4-d]pyrimidin-4 (3H)-one;
6-((2R,4S)-2-(1-cyclopropyl-1H-pyrazol-4-yl)tetrahydro-2H-pyran-4-yl)-8-(2,4-difluorophenyl)-3-methyl-2-(trifluoromethyl)pyrido[3,4-d]pyrimidin-4 (3H)-onedimethylpyrido[3,4-d]pyrimidin-4 (3H)-one;
5-chloro-2-(6-(2-(1-cyclopropyl-1H-pyrazol-4-yl)-6-methylmorpholino)-2,3-dimethyl-4-oxo-3,4-dihydropyrido[3,4-d]pyrimidin-8-yl)benzonitrile;
2,3-dimethyl-6-((2S,6R)-2-methyl-6-(2-methylpyridin-4-yl)morpholino)-8-(6-(trifluoromethyl)pyridin-3-yl)pyrido[3,4-d]pyrimidin-4 (3H)-one;
2,3-dimethyl-6-((2R,6S)-2-methyl-6-(2-methylpyridin-4-yl)morpholino)-8-(6-(trifluoromethyl)pyridin-3-yl)pyrido[3,4-d]pyrimidin-4 (3H)-one;
4-(6-((2R,6S)-2-(1-cyclopropyl-1H-pyrazol-4-yl)-6-methylmorpholino)-2,3-dimethyl-4-oxo-3,4-dihydropyrido[3,4-d]pyrimidin-8-yl)-3-fluorobenzonitrile;
4-(6-((2S,6R)-2-(1-cyclopropyl-1H-pyrazol-4-yl)-6-methylmorpholino)-2,3-dimethyl-4-oxo-3,4-dihydropyrido[3,4-d]pyrimidin-8-yl)-3-fluorobenzonitrile;
2-(6-((2R,6S)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-fluorobenzonitrile;
2-(6-((2S,6R)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-fluorobenzonitrile;
2-(6-((2R,6S)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-(trifluoromethyl)benzonitrile;
2-(6-((2S,6R)-2-(1-cyclopropyl-1H-pyrazol-4-yl)-6-methylmorpholino)-2,3-dimethyl-4-oxo-3,4-dihydropyrido[3,4-d]pyrimidin-8-yl)-5-(trifluoromethyl)benzonitrile;
(R)-8-(4-chloro-2-fluorophenyl)-2-methyl-6-(2-(1-methyl-1H-pyrazol-4-yl)morpholino)pyrimido[5,4-d]pyrimidin-4 (3H)-one; and
(S)-8-(4-chloro-2-fluorophenyl)-2-methyl-6-(2-(1-methyl-1H-pyrazol-4-yl)morpholino)pyrimido[5,4-d]pyrimidin-4 (3H)-one,
or a pharmaceutically acceptable salt thereof.

369. The compound according to any one of the preceding claims, or a pharmaceutically acceptable salt thereof, wherein the compound is a TREM2 modulator, such as a TREM2 activator, such as a TREM2 agonist.

370. The compound according to any one of the preceding claims, or a pharmaceutically acceptable salt thereof, wherein the compound enhances or activates TREM2 signaling through DAP12; and/or wherein the compound induces phosphorylation of a kinase that interacts with the TREM2/DAP12 signaling complex, such as, but not limited to, Syk, ZAP70, PI3K, Erk, AKT and GSK3b; and/or wherein the compound enhances TREM2-induced phosphorylation levels of the Syk kinase.

371. The compound according to any one of the preceding claims, or a pharmaceutically acceptable salt thereof, wherein the compound increases the expression of one or more TREM2 regulated genes, such as wherein the compound increases the expression of one or more TREM2 regulated genes selected from the group consisting of CXCL10, CCL2, CST7 and TMEM119.

372. A pharmaceutical composition comprising a compound according to any one of the preceding claims, or a pharmaceutically acceptable salt thereof, and one or more pharmaceutically acceptable carriers, excipients and/or diluents.

373. The compound according to any one of claims 1 to 371, or a pharmaceutically acceptable salt thereof, or the pharmaceutical composition according to claim 372, for use as a medicament.

374. The compound according to any one of claims 1 to 371, or a pharmaceutically acceptable salt thereof, or the pharmaceutical composition according to claim 372, for use in the treatment of a condition associated with a loss of function of TREM2, such as for use in the treatment of a condition associated with a mutation of TREM2.

375. The compound according to any one of claims 1 to 371, or a pharmaceutically acceptable salt thereof, or the pharmaceutical composition according to claim 372, for use in the treatment of a neurodegenerative disease.

376. The compound for use according to any one of claims 1 to 371368, or a pharmaceutically acceptable salt thereof, or the pharmaceutical composition according to claim 372, wherein said neurodegenerative disease is selected from the group consisting of a tauopathy, a TDP-43 proteinopathy, a synucleinopathy, dementia, amyloidosis, a demyelinating disorder of the CNS, a demyelinating disorder of the PNS, a Leukoencephalopathy, a leukodystrophy, a transmissible spongiform encephalopathy (TSE) and a lysosomal storage disorder (LSD).

377. The compound for use according to any one of claims 1 to 371, or a pharmaceutically acceptable salt thereof, or the pharmaceutical composition according to claim 372, for use in the treatment of a neurodegenerative disease selected from the group consisting of Alzheimer's disease, Frontotemporal lobar degeneration (FTLD), frontotemporal dementia (FTD), Parkinson's disease, Nasu-Hakola disease, FTLD-like syndrome, Huntington disease, Amyotrophic lateral sclerosis, multiple sclerosis, Guillain-Barre syndrome, chronic inflammatory demyelinating polyneuropathies, Charcot-Marie-Tooth disease, prion disease and stroke.

378. The compound for use according to any one of claims 1 to 371, or a pharmaceutically acceptable salt thereof, or the pharmaceutical composition according to claim 372, for use in the treatment of a disease selected from the group consisting of arthritis, rheumatoid arthritis, pyle disease, osteoporosis, osteopetrosis, osteosclerosis, skeletal dysplasia, dysosteoplasia, autism spectrum disorders, autism and Aspergers syndrome, traumatic brain injuries (TBI), spinal cord injuries, muscular dystrophy, myotonic dystrophy, inclusion-body myositis, systemic lupus erythematosus (SLE), RA, gout, bowel conditions, Inflammatory bowel disease (IBD), metabolic syndrome, obesity, type 2 diabetes, atherosclerosis, alcoholic and non-alcoholic fatty liver disease, alcoholic and non-alcoholic steatohepatitis, Amyloidosis.

379. The compound for use according to any one of claims 1 to 371, or a pharmaceutically acceptable salt thereof, or the pharmaceutical composition according to claim 372, wherein said compound is administered in an amount of about 0.01 mg/kg to about 100 mg/kg bodyweight/day.

380. The compound for use according to any one of claims 1 to 371, or a pharmaceutically acceptable salt thereof, or the pharmaceutical composition according to claim 372, wherein said compound is administered via enteral delivery, oral delivery, topical delivery, parenteral delivery, intravenous delivery, intradermal delivery, intramuscular delivery, intrathecal delivery, colonic delivery, rectal delivery, or intraperitoneal delivery.

381. A method for treatment of a condition associated with a loss of function of TREM2, such as a neurodegenerative disease, said method comprising administering a therapeutically effective amount of a compound according to any one of claims 1 to 371, or a pharmaceutically acceptable salt thereof, or the pharmaceutical composition according to claim 372, to a subject in need thereof.

382. Use of a compound according to any one of claims 1 to 371, or a pharmaceutically acceptable salt thereof, or the pharmaceutical composition according to claim 372, for the manufacture of a medicament for the treatment of a condition associated with a loss of function of TREM2, such as a neurodegenerative disease.

383. A method of enhancing or increasing TREM2 activity, such as a method of one or more of i) enhancing or activating TREM2 signaling through DAP12, ii) inducing phosphorylation of a kinase that interacts with the TREM2/DAP12 signaling complex, such as, but not limited to, Syk, ZAP70, PI3K, Erk, AKT and GSK3b, iii) enhancing TREM2-induced phosphorylation levels of the Syk kinase, Iv) increasing the expression levels, such as brain expression levels, of one or more TREM2 regulated genes, and/or v) increasing the expression levels, such as brain expression levels, of one or more TREM2 regulated genes selected from the group consisting of CXCL10, CCL2, CST7 and TMEM119; in a subject in need thereof, such as in a subject having a neurodegenerative disease, said method comprising administering to the subject a compound, or a pharmaceutically acceptable salt thereof, according to any one of claims 1 to 371.

384. A method for manufacturing a compound of formula IX according to any one of claims 1 to 371 comprising the steps of:
a. reacting a compound of formula (R¹CO)₂O with 5-amino-2,6-dioxo-1,2,3,6-tetrahydropyrimidine-4-carboxylic acid to generate a compound of formula A1 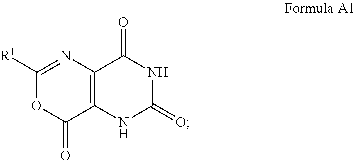
b. reacting the compound of formula A1 generated in a) with a compound of formula R²NH₂ to generate a compound of formula A2: 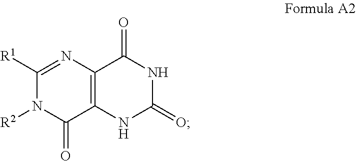
c. activating the compound of formula A2 generated in b) with POY₃, wherein Y is Cl or Br, to generate a compound of formula A3: 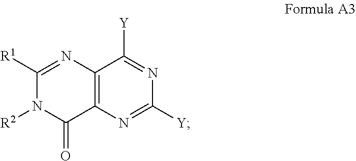
d. reacting the compound of formula A3 generated in c) with R³W¹, wherein W is B(OH)₂, OH or NH, to generate a compound of formula A4: 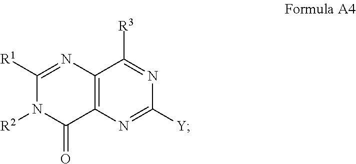
and
e. reacting the compound of formula A4 generated in d) with R²³W² wherein W is B(OH)₂, OH or NH, to generate a compound of formula IX.

385. A method for manufacturing a compound of formula B6, comprising the steps of:
a. reacting R¹COCl with a compound of formula B1 to form a compound of formula B2: 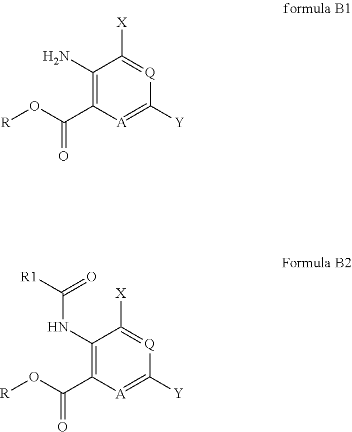
b. hydrolysing the ester of B2 to form a compound of formula B3: 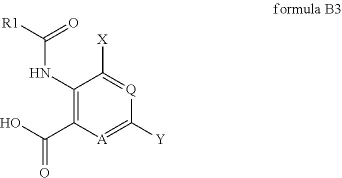
c. reacting the compound of formula B3 R²NH₂ to form a compound of formula B4: 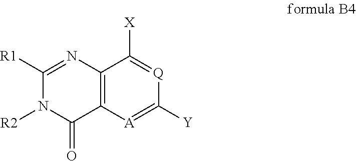
d. reacting the compound of formula B4 with R³W¹, wherein W¹ is B(OH)₂, OH or NH, to generate a compound of formula B5: 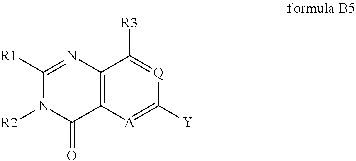
and
e. reacting the compound of formula B5 with R²³W², wherein W² is B(OH)₂, OH or NH, to generate a compound of formula B6: 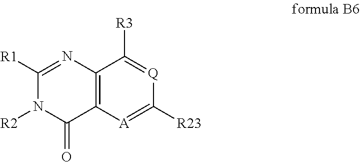
wherein R¹, R², R³ and R²³ are as defined in any one of claims 1 to 371 and R is alkyl, such as C₁₋₆ alkyl; A is N, CH or CF; Q is N or CH; X is Cl or Br; and Y is Cl or Br.

386. A method for manufacturing a compound of formula C4, comprising the steps of:
a. reacting a compound of formula C1 with an orthoacetate reagent (e.g. R¹C(OEt)₃) to form a compound of formula C2: 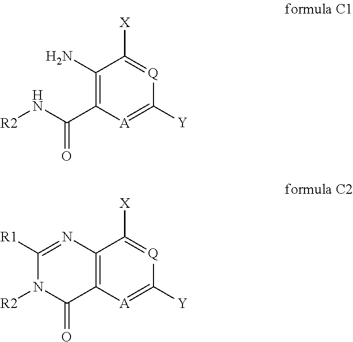
b. reacting the compound of formula C2 with R³W¹, wherein W¹ is B(OH)₂, OH or NH, to generate a compound of formula C3: 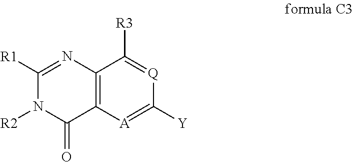
and
c. reacting the compound of formula C3 with R²³W², wherein W² is B(OH)₂, OH or NH, to generate a compound of formula C4: 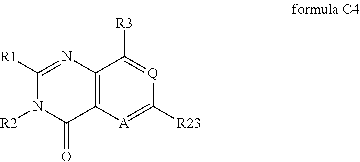
wherein R¹, R², R³ and R²³ are as defined in any one of claims 1 to 371 and A is CH, CF or N; Q is N or CH; X is Cl or Br; and Y is Cl or Br.

387. A method for manufacturing a compound of formula D6, comprising the steps of:
a. reacting a compound of formula D1 with R²³W², wherein W² is B(OH)₂, OH or NH, to generate a compound of formula D2: 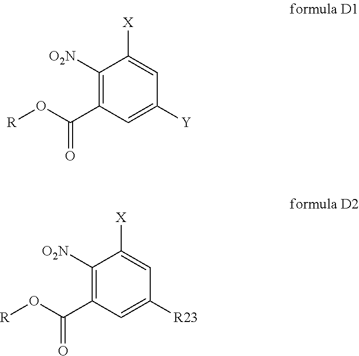
b. reacting the compound of formula D2 with R³W¹, wherein W¹ is B(OH)₂, OH or NH, to generate a compound of formula D3: 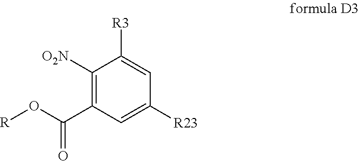
c. reducing and hydrolysing the compound of formula D3 to generate a compound of formula D4: 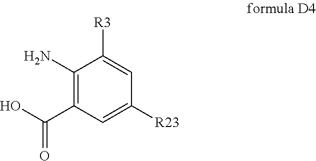
d. reacting the compound of formula D4 with (R¹CO)₂O to generate a compound of formula D5: 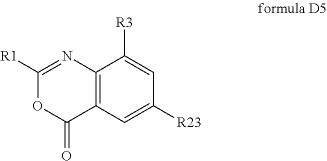
and
e. reacting the compound of formula D5 with R²NH₂ to generate a compound of formula D6: 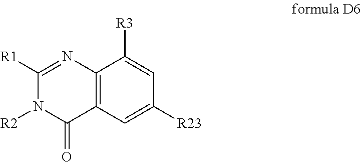
wherein R¹, R², R³ and R²³ are as defined in any one of claims 1 to 371 and X is Cl or Br; and Y is Cl or Br, and R is alkyl, such as C₁₋₆ alkyl.

388. A method for manufacturing a compound of formula E7, comprising the steps of:
a. reacting a compound of formula E1 with R¹CCH to generate a compound of formula E2: 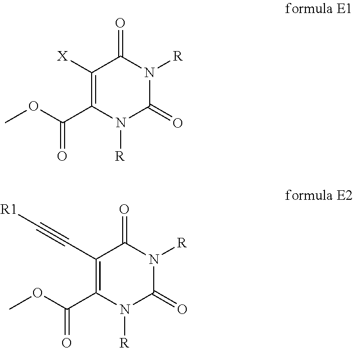
b. hydrolysing the compound of formula E2 to generate a compound of formula E3: 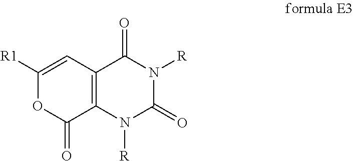
c. reacting the compound of formula E3 with R²NH₂ and removing the R-group to generate a compound of formula E4: 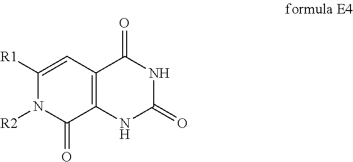
d. reacting the compound of formula E4 with POCl₃ or POBr₃ to generate a compound of formula E5: 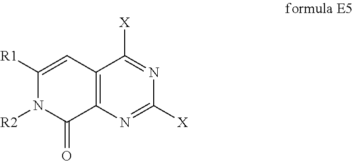
e. reacting the compound of formula E5 with R³W¹, wherein W¹ is B(OH)₂, OH or NH, to generate a compound of formula E6: 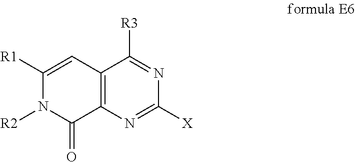
and
f. reacting the compound of formula E6 with R²³W², wherein W² is B(OH)₂, OH or NH, to generate a compound of formula E7: 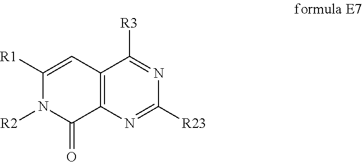
wherein R¹, R², R³ and R²³ are as defined in any one of claims 1 to 371 and R is a suitable protecting group such as PMB; and X is I, Cl or Br.
